SEQUENCE LISTING (459 sequences)

SEQ ID NO: 1 - DNA, 1419 bp, synthetic construct
  misc_feature 1..1419: note = Synthetic: gD.gD.mIgG2a.LALAPG.AV-Hole (heavy chain (HC) DNA including signal peptide (SP))
  source 1..1419: mol_type = other DNA
atgggatggtcatgtatcatcctttttctagtagcaactgcaaccggtgtacattcagaggttcagctggtggagtctggcggtggcctggtgcagccagggggctcactccgtttgtcctgtgccgcttctggctactccatcacctccgactttgcctggaactgggtccgtcaggccccgggtaagggcctggaatgggttggatacattagttactctggaaccactagctataaccctagcctgaagtcccgtatcactataagtcgcgacaattccaaaaacacattctacctgcagatgaacagcctgcgtgctgaggacactgccgtctattattgtgctcgagaaaactactatggccgttctcacgttgggtacttcgacgtctggggtcaaggaaccctggtcaccgtctcgagtgccaaaacaacagccccatcggtctatccactggctcctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacgtcgagcacctggcccagccagtccatcacctgcaatgtggcccacccggcaagcagcaccaaggtggacaagaaaattgagcccagaggacccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctacgcgtggtcagtgccctccccatccagcaccaggactggatgagtggcaaggagttcaaatgcaaggtcaacaacaaagacctcggagcgcccatcgagagaaccatctcaaaacccaaagggtcagtaagagctccacaggtatatgtcttgcctccaccagaagaagagatgactaagaaacaggtcactctgacctgcgctgtcacagacttcatgcctgaagacatttacgtggagtggaccaacaacgggaaaacagagctaaactacaagaacactgaaccagtcctggactctgatggttcttacttcatggttagcaagctgagagtggaaaagaagaactgggtggaaagaaatagctactcctgttcagtggtccacgagggtctgcacaatcaccacacgactaagagcttctcccggactccgggtaaatga

SEQ ID NO: 2 - DNA, 714 bp, synthetic construct
  misc_feature 1..714: note = Synthetic: gD.gD.mIgG2a.LALAPG.AV-Hole (light chain (LC) DNA including SP)
  source 1..714: mol_type = other DNA
atgggatggtcatgtatcatcctttttctagtagcaactgcaactggagtacattcagatatccagatgacccagtccccgagctccctgtccgcctctgtgggcgatagggtcaccatcacctgccgtgccagtgcgtctgtcgacagctatggtaacagcttcatccactggtatcagcagaaaccaggaaaagctccgaaactactgatttaccgggcctcggacctggagtctggagtcccttctcgcttctctggatccggttctgggacggatttcactctgaccatcagcagtctgcagccagaagacttcgcaacttattactgtcagcaaaattacgcggatccgttcacatttggacagggtaccaaggtggagatcaagcgcgctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 3 - protein, 453 aa, synthetic construct
  REGION 1..453: note = Synthetic: gD.gD.mIgG2a.LALAPG.AV-Hole heavy chain (mature sequence)
EVQLVESGGGLVQPGGSLRLSCAASGYSITSDFAWNWVRQAPGKGLEWVGYISYSGTTSYNPSLKSRITISRDNSKNTFYLQMNSLRAEDTAVYYCARENYYGRSHVGYFDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 4 - protein, 218 aa, synthetic construct
  REGION 1..218: note = Synthetic: gD.gD.mIgG2a.LALAPG.AV-Hole light chain (mature sequence)
DIQMTQSPSSLSASVGDRVTITCRASASVDSYGNSFIHWYQQKPGKAPKLLIYRASDLESGVPSRFSGSGSGTDFTLTISSLQPEDFATYYCQQNYADPFTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - DNA, 1389 bp, synthetic construct
  misc_feature 1..1389: note = Synthetic: xRNF43.hSC37.39.mIgG2a.LALAPG.AV-Hole.AX (HC DNA with SP)
  source 1..1389: mol_type = other DNA
atgggatggtcatgtatcatcctttttctagtagcaactgcaaccggtgtacattcacaggtgcagctggtgcagtccggcgccgaggtgaagaagcccggcgcctccgtgaaggtgtcctgcaaggcctccggctacaccttcaccacctacaccatccactgggtgaggcaggcccccggccagggcctggagtggatgggctacatcaaccccaggtccggctacaccgagtacaaccagaagttccaggacagggtgaccatgaccagggacacctccacctccaccgtgtacatggagctgtcctccctgaggtccgaggacaccgccgtgtactactgcgccaggtcctacgagttctggggccagggcaccaccgtgaccgtctcgagtgccaaaacaacagccccatcggtctatccactggctcctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacgtcgagcacctggcccagccagtccatcacctgcaatgtggcccacccggcaagcagcaccaaggtggacaagaaaattgagcccagaggacccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctacgcgtggtcagtgccctccccatccagcaccaggactggatgagtggcaaggagttcaaatgcaaggtcaacaacaaagacctcggagcgcccatcgagagaaccatctcaaaacccaaagggtcagtaagagctccacaggtatatgtcttgcctccaccagaagaagagatgactaagaaacaggtcactctgacctgcgctgtcacagacttcatgcctgaagacatttacgtggagtggaccaacaacgggaaaacagagctaaactacaagaacactgaaccagtcctggactctgatggttcttacttcatggttagcaagctgagagtggaaaagaagaactgggtggaaagaaatagctactcctgttcagtggtccacgagggtctgcacaatcaccacacgactaagagcttctcccggactccgggtaaatga

SEQ ID NO: 6 - DNA, 701 bp, synthetic construct
  misc_feature 1..701: note = Synthetic: xRNF43.hSC37.39.mIgG2a.LALAPG.AV-Hole.AX (LC DNA with SP)
  source 1..701: mol_type = other DNA
atgggatggtcatgtatcatcctttttctagtagcaactgcaaccggtgtacattcagagatcgtgatgacccagtcccccgccaccctgtccgtgtcccccggcgagagggccaccctgtcctgcaaggcctcccagaacgtgggcatcaacgtggcctggtatcagcagaagcccggccaggcccccagggccctgatctactccgcctcctacaggtactccggcatccccgccaggttctccggctccggctccggcaccgagttcaccctgaccatctcctccctgcagtccgaggacttcgccgtgtactactgccaccagtacaagacctacccctacaccttcggcggcggtaccaagctggagatcaagcgcgctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttg

SEQ ID NO: 7 - protein, 443 aa, synthetic construct
  REGION 1..443: note = Synthetic: xRNF43.hSC37.39.mIgG2a.LALAPG.AV-Hole.AX mature HC
EVQLVQSGAEVKKPGASVKVSCKASGYTFTTYTIHWVRQAPGQGLEWMGYINPRSGYTEYNQKFQDRVTMTRDTSTSTVYMELSSLRSEDTAVYYCARSYEFWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 8 - protein, 214 aa, synthetic construct
  REGION 1..214: note = Synthetic: xRNF43.hSC37.39.mIgG2a.LALAPG.AV-Hole.AX mature LC
EIVMTQSPATLSVSPGERATLSCKASQNVGINVAWYQQKPGQAPRALIYSASYRYSGIPARFSGSGSGTEFTLTISSLQSEDFAVYYCHQYKTYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - DNA, 1410 bp, synthetic construct
  misc_feature 1..1410: note = Synthetic: NIST.mIgG2a.LALAPG.AV-Hole (HC DNA with SP)
  source 1..1410: mol_type = other DNA
atgggatggtcatgtatcatcctttttctagtagcaactgcaaccggtgtacattcacaggtgaccctgagggagtccggccccgccctggtgaagcccacccagaccctgaccctgacctgcaccttctccggcttctccctgtccaccgccggcatgtccgtgggctggatcaggcagccccccggcaaggccctggagtggctggccgacatctggtgggacgacaagaagcactacaacccctccctgaaggacaggctgaccatctccaaggacacctccaagaaccaggtggtgctgaaggtgaccaacatggaccccgccgacaccgccacctactactgcgccagggacatgatcttcaacttctacttcgacgtgtggggccagggcaccaccgtgaccgtctcgagtgccaaaacaacagccccatcggtctatccactggctcctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacgtcgagcacctggcccagccagtccatcacctgcaatgtggcccacccggcaagcagcaccaaggtggacaagaaaattgagcccagaggacccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctacgcgtggtcagtgccctccccatccagcaccaggactggatgagtggcaaggagttcaaatgcaaggtcaacaacaaagacctcggagcgcccatcgagagaaccatctcaaaacccaaagggtcagtaagagctccacaggtatatgtcttgcctccaccagaagaagagatgactaagaaacaggtcactctgacctgcgctgtcacagacttcatgcctgaagacatttacgtggagtggaccaacaacgggaaaacagagctaaactacaagaacactgaaccagtcctggactctgatggttcttacttcatggttagcaagctgagagtggaaaagaagaactgggtggaaagaaatagctactcctgttcagtggtccacgagggtctgcacaatcaccacacgactaagagcttctcccggactccgggtaaatga

SEQ ID NO: 10 - DNA, 699 bp, synthetic construct
  misc_feature 1..699: note = Synthetic: NIST.mIgG2a.LALAPG.AV-Hole (LC DNA with SP)
  source 1..699: mol_type = other DNA
atgggatggtcatgtatcatcctttttctagtagcaactgcaaccggtgtacattcagacatccagatgacccagtccccctccaccctgtccgcctccgtgggcgacagggtgaccatcacctgctccgcctcctccagggtgggctacatgcactggtatcagcagaagcccggcaaggcccccaagctgctgatctacgacacctccaagctggcctccggcgtgccctccaggttctccggctccggctccggcaccgagttcaccctgaccatctcctccctgcagcccgacgacttcgccacctactactgcttccagggctccggctaccccttcaccttcggcggcggtaccaaggtggagatcaagcgcgctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 11 - protein, 449 aa, synthetic construct
  REGION 1..449: note = Synthetic: NIST.mIgG2a.LALAPG.AV-Hole (HC mature)
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCAVTDFMPEDIYVEWTNNGKTELNYKNTEPVLDSDGSYFMVSKLRVEKKNWVERNSYSCSVVHEGLHNHHTTKSFSRTPG

SEQ ID NO: 12 - protein, 213 aa, synthetic construct
  REGION 1..213: note = Synthetic: NIST.mIgG2a.LALAPG.AV-Hole (LC mature)
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 13 - DNA, 2088 bp, synthetic construct
  misc_feature 1..2088: note = Synthetic: HER2.4D5VH-mIgG2aCH1-4D5VH.mIgG2a.LALAPG.Knob (HC DNA with SP)
  source 1..2088: mol_type = other DNA
atgggatggtcatgtatcatcctttttctagtagcaactgcaaccggtgtacattcagaagttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcgagtgccaagaccaccgcccccagcgtgtaccccctggcccccgtgtgcggcgacaccaccggcagcagcgtgaccctgggctgcctggtgaagggctacttccccgagcccgtgaccctgacctggaacagcggcagcctgagcagcggcgtgcacaccttccccgccgtgctgcagagcgacctgtacaccctgagcagcagcgtgaccgtgaccagcagcacctggcccagccagagcatcacctgcaacgtggcccaccccgccagcagcaccaaggtggacaagaagatcgagcccagaggccccaccatcaagcccgaggtgcagctggtggagagcggcggcggcctggtgcagcccggcggcagcctgagactgagctgcgccgccagcggcttcaacatcaaggacacctacatccactgggtgagacaggcccccggcaagggcctggagtgggtggccagaatctaccccaccaacggctacaccagatacgccgacagcgtgaagggcagattcaccatcagcgccgacaccagcaagaacaccgcctacctgcagatgaacagcctgagagccgaggacaccgccgtgtactactgcagcagatggggcggcgacggcttctacgccatggactactggggccagggcaccctggtgaccgtctcgagtgccaaaacaacagccccatcggtctatccactggctcctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacgtcgagcacctggcccagccagtccatcacctgcaatgtggcccacccggcaagcagcaccaaggtggacaagaaaattgagcccagaggacccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctacgcgtggtcagtgccctccccatccagcaccaggactggatgagtggcaaggagttcaaatgcaaggtcaacaacaaagacctcggagcgcccatcgagagaaccatctcaaaacccaaagggtcagtaagagctccacaggtatatgtcttgcctccaccagaagaagagatgactaagaaacaggtcactctgtggtgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 14 - DNA, 702 bp, synthetic construct
  misc_feature 1..702: note = Synthetic: HER2.4D5VH-mIgG2aCH1-4D5VH.mIgG2a.LALAPG.Knob (LC DNA with SP)
  source 1..702: mol_type = other DNA
atgggatggtcatgtatcatcctttttctagtagcaactgcaaccggtgtacattcagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcactctgaccatcagcagtctgcagccggaagacttcgcaacttattactgtcagcaacattatactactcctcccacgttcggacagggtaccaagctggagatcaagcgcgctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 15 - protein, 676 aa, synthetic construct
  REGION 1..676: note = Synthetic: HER2.4D5VH-mIgG2aCH1-4D5VH.mIgG2a.LALAPG.Knob (HC mature)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 16 - protein, 214 aa, synthetic construct
  REGION 1..214: note = Synthetic: HER2.4D5VH-mIgG2aCH1-4D5VH.mIgG2a.LALAPG.Knob (LC mature)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 17 - DNA, 2082 bp, synthetic construct
  misc_feature 1..2082: note = Synthetic: HER2.2C4VH-mIgG2aCH1-2C4VH.mIgG2a.LALAPG.Knob (HC DNA with SP)
  source 1..2082: mol_type = other DNA
atgggatggtcatgtatcatcctttttctagtagcaactgcaaccggtgtacattcagaggtgcagctggtggagagcggcggcggcctggtgcagccaggcggcagcctgcgcctgagctgcgccgccagcggcttcaccttcaccgactacaccatggactgggtgcgccaggccccaggcaagggcctggagtgggtggccgacgtgaacccaaacagcggcggcagcatctacaaccagcgcttcaagggccgcttcaccctgagcgtggaccgcagcaagaacaccctgtacctgcagatgaacagcctgcgcgccgaggacaccgccgtgtactactgcgcccgcaacctgggcccaagcttctacttcgactactggggccagggcaccctggtgaccgtctcgagtgccaagaccaccgcccccagcgtgtaccccctggcccccgtgtgcggcgacaccaccggcagcagcgtgaccctgggctgcctggtgaagggctacttccccgagcccgtgaccctgacctggaacagcggcagcctgagcagcggcgtgcacaccttccccgccgtgctgcagagcgacctgtacaccctgagcagcagcgtgaccgtgaccagcagcacctggcccagccagagcatcacctgcaacgtggcccaccccgccagcagcaccaaggtggacaagaagatcgagcccagaggccccaccatcaagcccgaggtgcagctggtggagagcggcggcggcctggtgcagcccggcggcagcctgagactgagctgcgccgccagcggcttcaccttcaccgactacaccatggactgggtgagacaggcccccggcaagggcctggagtgggtggccgacgtgaaccccaacagcggcggcagcatctacaaccagagattcaagggcagattcaccctgagcgtggacagaagcaagaacaccctgtacctgcagatgaacagcctgagagccgaggacaccgccgtgtactactgcgccagaaacctgggccccagcttctacttcgactactggggccagggcaccctggtgaccgtctcgagtgccaaaacaacagccccatcggtctatccactggctcctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacgtcgagcacctggcccagccagtccatcacctgcaatgtggcccacccggcaagcagcaccaaggtggacaagaaaattgagcccagaggacccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctacgcgtggtcagtgccctccccatccagcaccaggactggatgagtggcaaggagttcaaatgcaaggtcaacaacaaagacctcggagcgcccatcgagagaaccatctcaaaacccaaagggtcagtaagagctccacaggtatatgtcttgcctccaccagaagaagagatgactaagaaacaggtcactctgtggtgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 18 - DNA, 702 bp, synthetic construct
  misc_feature 1..702: note = Synthetic: HER2.2C4VH-mIgG2aCH1-2C4VH.mIgG2a.LALAPG.Knob (LC DNA with SP)
  source 1..702: mol_type = other DNA
atgggatggtcatgtatcatcctttttctagtagcaactgcaaccggtgtacattcagacatccagatgacccagagcccaagcagcctgagcgccagcgtgggcgaccgcgtgaccatcacctgcaaggccagccaggacgtgagcatcggcgtggcctggtatcagcagaagccaggcaaggccccaaagctgctgatctacagcgccagctaccgctacaccggcgtgccaagccgcttcagcggcagcggcagcggcaccgacttcaccctgaccatcagcagcctgcagccagaggacttcgccacctactactgccagcagtactacatctacccatacaccttcggccagggtaccaagctggagatcaagcgcgctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 19 - protein, 674 aa, synthetic construct
  REGION 1..674: note = Synthetic: HER2.2C4VH-mIgG2aCH1-2C4VH.mIgG2a.LALAPG.Knob (HC mature)
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 20 - protein, 214 aa, synthetic construct
  REGION 1..214: note = Synthetic: HER2.2C4VH-mIgG2aCH1-2C4VH.mIgG2a.LALAPG.Knob (LC mature)
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - DNA, 2076 bp, synthetic construct
  misc_feature 1..2076: note = Synthetic: HER2.7C2VH-mIgG2aCH1-7C2VH.mIgG2a.LALAPG.Knob (HC DNA with SP)
  source 1..2076: mol_type = other DNA
atgggatggtcatgtatcatcctttttctagtagcaactgcaaccggtgtacattcacaggtccaactgcagcagcctggggctgaactggtgaggcctggagcttcagtgaagctgtcctgcaaggcttctggctactccttcaccggctactggatgaactggttgaagcagaggcctggacaaggccttgagtggattggcatgattcatccttccgatagtgaaattagggcaaatcagaaattcagggacaaggccacattgactgtagacaagtcctccaccacagcctacatgcaactcagcagcccgacatctgaggactctgcggtctattactgtgcacgagggacttacgacggaggctttgaatactggggccaaggcaccactctcacagtctcgagtgccaagaccaccgcccccagcgtgtaccccctggcccccgtgtgcggcgacaccaccggcagcagcgtgaccctgggctgcctggtgaagggctacttccccgagcccgtgaccctgacctggaacagcggcagcctgagcagcggcgtgcacaccttccccgccgtgctgcagagcgacctgtacaccctgagcagcagcgtgaccgtgaccagcagcacctggcccagccagagcatcacctgcaacgtggcccaccccgccagcagcaccaaggtggacaagaagatcgagcccagaggccccaccatcaagccccaggtgcagctgcagcagcccggcgccgagctggtgagacccggcgccagcgtgaagctgagctgcaaggccagcggctacagcttcaccggctactggatgaactggctgaagcagagacccggccagggcctggagtggatcggcatgatccaccccagcgacagcgagatcagagccaaccagaagttcagagacaaggccaccctgaccgtggacaagagcagcaccaccgcctacatgcagctgagcagccccaccagcgaggacagcgccgtgtactactgcgccagaggcacctacgacggcggcttcgagtactggggccagggcaccaccctgaccgtctcgagtgccaaaacaacagccccatcggtctatccactggctcctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacgtcgagcacctggcccagccagtccatcacctgcaatgtggcccacccggcaagcagcaccaaggtggacaagaaaattgagcccagaggacccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctacgcgtggtcagtgccctccccatccagcaccaggactggatgagtggcaaggagttcaaatgcaaggtcaacaacaaagacctcggagcgcccatcgagagaaccatctcaaaacccaaagggtcagtaagagctccacaggtatatgtcttgcctccaccagaagaagagatgactaagaaacaggtcactctgtggtgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 22 - DNA, 717 bp, synthetic construct
  misc_feature 1..717: note = Synthetic: HER2.7C2VH-mIgG2aCH1-7C2VH.mIgG2a.LALAPG.Knob (LC DNA with SP)
  source 1..717: mol_type = other DNA
atgggatggtcatgtatcatcctttttctagtagcaactgcaaccggtgtacattcagacatcgtgctgacccagagcccagccagcctggtggtgagcctgggccagcgcgccaccatcagctgccgcgccagccagagcgtgagcggcagccgcttcacctacatgcactggtatcagcagaagccaggccagccaccaaagctgctgatcaagtacgccagcatcctggagagcggcgtgccagcccgcttcagcggcggcggcagcggcaccgacttcaccctgaacatccacccagtggaggaggacgacaccgccacctactactgccagcacagctgggagatcccaccatggaccttcggcggcggtaccaagctggagatcaagcgcgctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 23 - protein, 672 aa, synthetic construct
  REGION 1..672: note = Synthetic: HER2.7C2VH-mIgG2aCH1-7C2VH.mIgG2a.LALAPG.Knob (HC mature)
QVQLQQPGAELVRPGASVKLSCKASGYSFTGYWMNWLKQRPGQGLEWIGMIHPSDSEIRANQKFRDKATLTVDKSSTTAYMQLSSPTSEDSAVYYCARGTYDGGFEYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPQVQLQQPGAELVRPGASVKLSCKASGYSFTGYWMNWLKQRPGQGLEWIGMIHPSDSEIRANQKFRDKATLTVDKSSTTAYMQLSSPTSEDSAVYYCARGTYDGGFEYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 24 - protein, 219 aa, synthetic construct
  REGION 1..219: note = Synthetic: HER2.7C2VH-mIgG2aCH1-7C2VH.mIgG2a.LALAPG.Knob (LC mature)
DIVLTQSPASLVVSLGQRATISCRASQSVSGSRFTYMHWYQQKPGQPPKLLIKYASILESGVPARFSGGGSGTDFTLNIHPVEEDDTATYYCQHSWEIPPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 25 - DNA, 1440 bp, synthetic construct
  misc_feature 1..1440: note = Synthetic: IGF-1R.xIGF-1RcixutumumabVH.mIgG2a.LALAPG.Knob (HC DNA with SP)
  source 1..1440: mol_type = other DNA
atgggatggtcatgtatcatcctttttctagtagcaactgcaaccggtgtacattcagaggtgcagctggtgcagagcggcgccgaggtgaagaagcccggcagcagcgtgaaggtgagctgcaaggccagcggcggcaccttcagcagctacgccatcagctgggtgagacaggcccccggccagggcctggagtggatgggcggcatcatccccatcttcggcaccgccaactacgcccagaagttccagggcagagtgaccatcaccgccgacaagagcaccagcaccgcctacatggagctgagcagcctgagaagcgaggacaccgccgtgtactactgcgccagagcccccctgagattcctggagtggagcacccaggaccactactactactactacatggacgtgtggggcaagggcaccaccgtgaccgtctcgagtgccaaaacaacagccccatcggtctatccactggctcctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacgtcgagcacctggcccagccagtccatcacctgcaatgtggcccacccggcaagcagcaccaaggtggacaagaaaattgagcccagaggacccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctacgcgtggtcagtgccctccccatccagcaccaggactggatgagtggcaaggagttcaaatgcaaggtcaacaacaaagacctcggagcgcccatcgagagaaccatctcaaaacccaaagggtcagtaagagctccacaggtatatgtcttgcctccaccagaagaagagatgactaagaaacaggtcactctgtggtgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 26 - DNA, 699 bp, synthetic construct
  misc_feature 1..699: note = Synthetic: IGF-1R.xIGF-1RcixutumumabVH.mIgG2a.LALAPG.Knob (LC DNA with SP)
  source 1..699: mol_type = other DNA
atgggatggtcatgtatcatcctttttctagtagcaactgcaaccggtgtacattcaagcagcgagctgacccaggaccccgccgtgagcgtggccctgggccagaccgtgagaatcacctgccagggcgacagcctgagaagctactacgccacctggtaccagcagaagcccggccaggcccccatcctggtgatctacggcgagaacaagagacccagcggcatccccgacagattcagcggcagcagcagcggcaacaccgccagcctgaccatcaccggcgcccaggccgaggacgaggccgactactactgcaagagcagagacggcagcggccagcacctggtgttcggcggcggcaccaagctgaccgtgcttggccagcccaagtccactcccactctcaccgtgtttccaccctcgagtgaggagctcaaggaaaacaaagccacactggtgtgtctgatttccaacttttccccgagtggtgtgacagtggcctggaaggcaaatggtacacctatcacccagggtgtggacacttcaaatcccaccaaagagggcaacaagttcatggcgagcagcttcctacatttgacatcggaccagtggagatctcacaacagttttacctgtcaagttacacatgaaggggacactgtggagaagagtctgtcacgtgctgactgtctctga

SEQ ID NO: 27 - protein, 460 aa, synthetic construct
  REGION 1..460: note = Synthetic: IGF-1R.xIGF-1RcixutumumabVH.mIgG2a.LALAPG.Knob (HC mature)
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 28 - protein, 213 aa, synthetic construct
  REGION 1..213: note = Synthetic: IGF-1R.xIGF-1RcixutumumabVH.mIgG2a.LALAPG.Knob (LC mature)
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLGQPKSTPTLTVFPPSSEELKENKATLVCLISNFSPSGVTVAWKANGTPITQGVDTSNPTKEGNKFMASSFLHLTSDQWRSHNSFTCQVTHEGDTVEKSLSRADCL

SEQ ID NO: 29 - DNA, 1410 bp, synthetic construct
  misc_feature 1..1410: note = Synthetic: NIST.mIgG2a.LALAPG.Knob (HC DNA with SP)
  source 1..1410: mol_type = other DNA
atgggatggtcatgtatcatcctttttctagtagcaactgcaaccggtgtacattcacaggtgaccctgagggagtccggccccgccctggtgaagcccacccagaccctgaccctgacctgcaccttctccggcttctccctgtccaccgccggcatgtccgtgggctggatcaggcagccccccggcaaggccctggagtggctggccgacatctggtgggacgacaagaagcactacaacccctccctgaaggacaggctgaccatctccaaggacacctccaagaaccaggtggtgctgaaggtgaccaacatggaccccgccgacaccgccacctactactgcgccagggacatgatcttcaacttctacttcgacgtgtggggccagggcaccaccgtgaccgtctcgagtgccaaaacaacagccccatcggtctatccactggctcctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacgtcgagcacctggcccagccagtccatcacctgcaatgtggcccacccggcaagcagcaccaaggtggacaagaaaattgagcccagaggacccacaatcaagccctgtcctccatgcaaatgcccagcacctaacgccgcgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctacgcgtggtcagtgccctccccatccagcaccaggactggatgagtggcaaggagttcaaatgcaaggtcaacaacaaagacctcggagcgcccatcgagagaaccatctcaaaacccaaagggtcagtaagagctccacaggtatatgtcttgcctccaccagaagaagagatgactaagaaacaggtcactctgtggtgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 30 - DNA, 699 bp, synthetic construct; identical to SEQ ID NO: 10
  misc_feature 1..699: note = Synthetic: NIST.mIgG2a.LALAPG.Knob (LC DNA with SP)
  source 1..699: mol_type = other DNA

SEQ ID NO: 31 - protein, 450 aa, synthetic construct
  REGION 1..450: note = Synthetic: NIST.mIgG2a.LALAPG.Knob (HC mature)
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 32 - protein, 213 aa, synthetic construct; identical to SEQ ID NO: 12
  REGION 1..213: note = Synthetic: NIST.mIgG2a.LALAPG.Knob (LC mature)

SEQ ID NO: 33 - DNA, 360 bp, synthetic construct
  misc_feature 1..360: note = Synthetic: RNF43-104 (variable heavy chain (VH) DNA)
  source 1..360: mol_type = other DNA
caggtgcagctgaaggagtcaggacctggcctggtgcagccctcacagaccctgtctctcacctgcagtgtctctgggttttcattaaccagcttccatgtgcactgggttcgacagcctccaggaaaaggtctggagtggatgggattaatgtggagtaatggagacacatcatataattcagatctcaaatcccgactgagcatcagcagggacacctccaagagtcaagtcttcttaaaaatgcacagtctgcagactgaagacacagccacttactactgtgccagaacagatgtataccacggattcggtgctatggatgcctggggtcagggaacttcagtcactgtctcctca

SEQ ID NO: 34 - DNA, 321 bp, synthetic construct
  misc_feature 1..321: note = Synthetic: RNF43-104 (variable light chain (VL) DNA)
  source 1..321: mol_type = other DNA
gacatccagatgacacagtctccgtcatttctgtctgcatctcttggaaacagcatcaccatcacttgccatgccagtcagttcatcaagggttggttagcctggtaccaacaaaagtcagggaatgctcctgaactgttaatttataagtcatctagcctgcattcaggggttccatcaagattcagtggcagcggatctggaacagattatattctcactatcagcaacctacagcctgaagatattgccacttattactgtcagcattatcaaggctttccgctcacgttcggttctgggaccaagctggagatcaaa

SEQ ID NO: 35 - protein, 120 aa, synthetic construct
  REGION 1..120: note = Synthetic: RNF43-104 (VH protein)
QVQLKESGPGLVQPSQTLSLTCSVSGFSLTSFHVHWVRQPPGKGLEWMGLMWSNGDTSYNSDLKSRLSISRDTSKSQVFLKMHSLQTEDTATYYCARTDVYHGFGAMDAWGQGTSVTVSS

SEQ ID NO: 36 - protein, 107 aa, synthetic construct
  REGION 1..107: note = Synthetic: RNF43-104 (VL protein)
DIQMTQSPSFLSASLGNSITITCHASQFIKGWLAWYQQKSGNAPELLIYKSSSLHSGVPSRFSGSGSGTDYILTISNLQPEDIATYYCQHYQGFPLTFGSGTKLEIK

SEQ ID NO: 37 - DNA, 363 bp, synthetic construct
  misc_feature 1..363: note = Synthetic: RNF43-106 (VH DNA)
  source 1..363: mol_type = other DNA
cagatccagttgggacagtctggacctgagctgacgaagcctggagagtcagtgaagatctcctgcaaggcttctgggtataccttctcagactatccaatacactgggtgaaacaggctccaggaaagggcttgaagtggatgggctggatcaacacctatactgggaagccaacatttgctgatgacttcaaaggacggtttgtcttctctttggaagcctctgccagcactgcaaatttgcagatcagcaacctcaaaaatgaggacacggctacatatttctgtgctctgggccccatttctacagtcttctatgctatggatgcctggggtcaaggaacttcagtcactgtctcctca

SEQ ID NO: 38 - DNA, 321 bp, synthetic construct
  misc_feature 1..321: note = Synthetic: RNF43-106 (VL DNA)
  source 1..321: mol_type = other DNA
gacatccagatgacccagtctccttcattcctgtctgcatctgtgggagacagagtcactatcaactgcaaagcaagtcagaatattaacaagtatttaaactggtatcagcaaaagcttggagaagctcccaaacgcctgatatataatacaaacaatttgcaaacaggcatcccatcaaggttcagtggcagtggatctggtacagattacacactcaccatcagcagcctgcagcctgaagattttgccacatatttctgcttgcagcataatagttttccgtacacgtttggagctgggaccaagctggaactgaaa

SEQ ID NO: 39 - protein, 121 aa, synthetic construct
  REGION 1..121: note = Synthetic: RNF43-106 (VH prot)
QIQLGQSGPELTKPGESVKISCKASGYTFSDYPIHWVKQAPGKGLKWMGWINTYTGKPTFADDFKGRFVFSLEASASTANLQISNLKNEDTATYFCALGPISTVFYAMDAWGQGTSVTVSS

SEQ ID NO: 40 - protein, 107 aa, synthetic construct
  REGION 1..107: note = Synthetic: RNF43-106 (VL prot)
DIQMTQSPSFLSASVGDRVTINCKASQNINKYLNWYQQKLGEAPKRLIYNTNNLQTGIPSRFSGSGSGTDYTLTISSLQPEDFATYFCLQHNSFPYTFGAGTKLELK

SEQ ID NO: 41 - DNA, 354 bp, synthetic construct
  misc_feature 1..354: note = Synthetic: RNF43-107 (VH DNA)
  source 1..354: mol_type = other DNA
gaggtgcagccggtggagtctgggggcggcttggtgcagcctggaaggtccctaaaactctcctgcccagcctcaggattcactttcagtaactatgacatggcctgggtccgccaggctccaacgaagggtctggagtgggtcgcttccattagtcctagtggtggtagaacttactatcgcgactccgtgaagggccgattcactatttccagagataattcaaaaagtaccctatacttgcaaatggacagtctgagatctgaggacaccgccactttttactgtgcaacacatcggtactacagttactttgatttctggggccaaggagtcatggtcacagtctcctca

SEQ ID NO: 42 - DNA, 321 bp, synthetic construct
  misc_feature 1..321: note = Synthetic: RNF43-107 (VL DNA)
  source 1..321: mol_type = other DNA
gacatccagatgacacagtctccagcttccctgtctgcatctctgggagaaactatctccatcgattgtcgaacaagtgaggacatttacaataatttagcgtggtatcagcagaagtcagggagatctcctcagctcctgatctctgctacaaataggttgcaagatggggtcccatcacggttcagtggcagtggttctggcacacagtattctctcaagatcagcggcatgcaacctgaagatgaaggggtttatttctgtctacagggttccaagtatccgtatacgtttggaactgggaccaagctggaactgaaa

SEQ ID NO: 43 - protein, 118 aa, synthetic construct
  REGION 1..118: note = Synthetic: RNF43-107 (VH prot)
EVQPVESGGGLVQPGRSLKLSCPASGFTFSNYDMAWVRQAPTKGLEWVASISPSGGRTYYRDSVKGRFTISRDNSKSTLYLQMDSLRSEDTATFYCATHRYYSYFDFWGQGVMVTVSS

SEQ ID NO: 44 - protein, 107 aa, synthetic construct
  REGION 1..107: note = Synthetic: RNF43-107(VL prot)
DIQMTQSPASLSASLGETISIDCRTSEDIYNNLAWYQQKSGRSPQLLISATNRLQDGVPSRFSGSGSGTQYSLKISGMQPEDEGVYFCLQGSKYPYTFGTGTKLELK

SEQ ID NO: 45 - DNA, 360 bp, synthetic construct
  misc_feature 1..360: note = Synthetic: RNF43-108 (VH DNA)
  source 1..360: mol_type = other DNA
cagatacaactgcagcaatctggggctgaactggtgaagcctgggtcctcagtgacagtttcctgcaaggcttctggctacaccttcaccagtaactttatgcactggataagacagcagcctggcaatggccttgagtggattgggtggatttatcctggcgatggtgatgcagagaccaatcgaaagttcaatgggaaggcaacactcactgcagacaaatcctccaccacagcctacttgcagctcagcagcctgacatctgaggactctgcagtctatttctgtgcaagaggagtgatgacgtcgttctggtactttgacttctggggcccaggaaccatagtcaccgtgtcctca

SEQ ID NO: 46 - DNA, 321 bp, synthetic construct
  misc_feature 1..321: note = Synthetic: RNF43-108 (VL DNA)
  source 1..321: mol_type = other DNA
gatgtccagatgacccagtctccatataatcttgctgcctctcctggagaaagtgtttccatcaattgcaaggcaagtaagagcattagtaagtatttagcctggtatcaacagaaacctgggaaaacaaataagattctaatctacgatgggtcaactttgcaatctggaattccatcgaggttcagtggcagtggatctggtacagatttcactctcaccatcagaggcctggagcctgaagattttggactctattactgtcaacagcataatgaacagccacctacgttcggctcggggacgaggttggaaataaaa

SEQ ID NO: 47 - protein, 120 aa, synthetic construct
  REGION 1..120: note = Synthetic: RNF43-108 (VH prot)
QIQLQQSGAELVKPGSSVTVSCKASGYTFTSNFMHWIRQQPGNGLEWIGWIYPGDGDAETNRKFNGKATLTADKSSTTAYLQLSSLTSEDSAVYFCARGVMTSFWYFDFWGPGTIVTVSS

SEQ ID NO: 48 - protein, 107 aa, synthetic construct
  REGION 1..107: note = Synthetic: RNF43-108(VL prot)
DVQMTQSPYNLAASPGESVSINCKASKSISKYLAWYQQKPGKTNKILIYDGSTLQSGIPSRFSGSGSGTDFTLTIRGLEPEDFGLYYCQQHNEQPPTFGSGTRLEIK

SEQ ID NO: 49 - DNA, 339 bp, synthetic construct
  misc_feature 1..339: note = Synthetic: RNF43-116 (VH DNA)
  source 1..339: mol_type = other DNA
caggtccagctgcagcagtctggagctgagctggcaaagcctggctcttcagtgaagatttcctgcaaggcttctggctacaccttcaccagctactatataagctggataaagcagacgactggacaggaccttgagtatattggatatgttaatacgagaagtggaggtactaactacaatgagaagttcaaggtcaaggccacattgactgtagacaattcctccagcacagccttcatgcaactcagcagcctgacacctgacgactctgcggtctattactgtgcaagaggcagaacggactggggccaaggcactctggtcactgtctcttca

SEQ ID NO: 50 - DNA, 318 bp, synthetic construct
  misc_feature 1..318: note = Synthetic: RNF43-116 (VL DNA)
  source 1..318: mol_type = other DNA
gacatccagatgacccagtctccttcactcctgtctgcatctgtgggagacagagtcactgtaaactgcaaagcaagtcagaatatttataagaacttagagtggtatcagcaaaagcatggagaagctccaaaactcctgatatattatacaaacaatttgcaaacgggcatctcatcaaggttcagtggcagtggatctggtacagattacacactcaccatcagcagcctgcagcctgaagatgttgccacatattactgctttcagtataacagcgggtacacgtttggagctgggaccaagctggaactgaaa

SEQ ID NO: 51 - protein, 113 aa, synthetic construct
  REGION 1..113: note = Synthetic: RNF43-116 (VH prot)
QVQLQQSGAELAKPGSSVKISCKASGYTFTSYYISWIKQTTGQDLEYIGYVNTRSGGTNYNEKFKVKATLTVDNSSSTAFMQLSSLTPDDSAVYYCARGRTDWGQGTLVTVSS

SEQ ID NO: 52 - protein, 106 aa, synthetic construct
  REGION 1..106: note = Synthetic: RNF43-116(VL prot)
DIQMTQSPSLLSASVGDRVTVNCKASQNIYKNLEWYQQKHGEAPKLLIYYTNNLQTGISSRFSGSGSGTDYTLTISSLQPEDVATYYCFQYNSGYTFGAGTKLELK

SEQ ID NO: 53 - DNA, 360 bp, synthetic construct
  misc_feature 1..360: note = Synthetic: RNF43-117 (VH DNA)
  source 1..360: mol_type = other DNA
caggtttctctgaaagagtctggccctgggatgttgcagccctcccagaccctcagtctgacttgcactttctctgagttttcactgagcacttatggtttgggtgtgggctggattcgtcagccttcagggaagggtctggagtggctggcgaacatttggtgggatgatgataagtactacaatccatctctgaaaaaccgcctcacaatctccaaggacacctccaacaaccaagcattcctcaagatcaccaatgtggacactgctgatactgccacgtactactgtgctcggatagacggcaggtggcactactttgattactggggccaaggagacagggtcatagtctcctca

SEQ ID NO: 54 - DNA, 336 bp, synthetic construct
  misc_feature 1..336: note = Synthetic: RNF43-117 (VL DNA)
  source 1..336: mol_type = other DNA
gatattgtcctgacccagactccatccatattgtctgctaccattggacaatcggtctccatctcttgcaggtcaactcagagtctcttagatagtgatggaaacacctatttatattggttcctacagaggccaggccagtctccacagcgtctaatttatttggtatccaacctgggatctggggtccccaacaggttcagtggcagtgggtcaggaacagatttcacactcaaaatcagtggagtggaggctgaggatttgggattttattactgcatgcaaggtgcccatactccgctcacgttcggttctgggaccaagctggaaatcaaa

SEQ ID NO: 55 - protein, 120 aa, synthetic construct
  REGION 1..120: note = Synthetic: RNF43-117 (VH prot)
QVSLKESGPGMLQPSQTLSLTCTFSEFSLSTYGLGVGWIRQPSGKGLEWLANIWWDDDKYYNPSLKNRLTISKDTSNNQAFLKITNVDTADTATYYCARIDGRWHYFDYWGQGDRVIVSS

SEQ ID NO: 56 - protein, 112 aa, synthetic construct
  REGION 1..112: note = Synthetic: RNF43-117(VL prot)
DIVLTQTPSILSATIGQSVSISCRSTQSLLDSDGNTYLYWFLQRPGQSPQRLIYLVSNLGSGVPNRFSGSGSGTDFTLKISGVEAEDLGFYYCMQGAHTPLTFGSGTKLEIK

SEQ ID NO: 57 - DNA, 363 bp, synthetic construct
  misc_feature 1..363: note = Synthetic: RNF43-123 (VH DNA)
  source 1..363: mol_type = other DNA
cagatccagttggtacagtctggacctgagctgaagaagcctggagagtcagtgaagatctcctgcaaggcttctgggtataccttcacagactatccaatgcactgggtgaaacaggctccaggaaagggcttgaagtggatgggctggatcaacacctatactgggaagccaacatatgctgatgacttcaaaggacggtttgtcttctctttggaagcctctgccaacactgcaaatttacagatcagcaacctcaaaaatgacgacacggctacatatttctgtgctctgggccccatttctacagtcttctatgctgtggatgcctggggtcaaggaacttcagtcactgtctcctca

SEQ ID NO: 58 - DNA, 321 bp, synthetic construct
  misc_feature 1..321: note = Synthetic: RNF43-123 (VL DNA)
  source 1..321: mol_type = other DNA
gacatccagatgacccagtctccttcattcctgtctgcatctgtgggagacagagtcactatcaactgcaaagcaagtcagaatattaacaagtacttaaactggtatcagcaaaaggttggagaagctcccaagcgcctgatgtataatacaaacaatttgcaaacaggcatcccatcaaggttcagtggcagtggatctggtacagattacacactcaccatcagcagcctgcagcctgaagattttgccacatatttctgcttgcagcataatagttttccgtacacgtttggacctgggaccgagctggaactgaaa

SEQ ID NO: 59 - protein, 121 aa, synthetic construct
  REGION 1..121: note = Synthetic: RNF43-123 (VH prot)
QIQLVQSGPELKKPGESVKISCKASGYTFTDYPMHWVKQAPGKGLKWMGWINTYTGKPTYADDFKGRFVFSLEASANTANLQISNLKNDDTATYFCALGPISTVFYAVDAWGQGTSVTVSS

SEQ ID NO: 60 - protein, 107 aa, synthetic construct
  REGION 1..107: note = Synthetic: RNF43-123(VL prot)
DIQMTQSPSFLSASVGDRVTINCKASQNINKYLNWYQQKVGEAPKRLMYNTNNLQTGIPSRFSGSGSGTDYTLTISSLQPEDFATYFCLQHNSFPYTFGPGTELELK

SEQ ID NO: 61 - DNA, 357 bp, synthetic construct
  misc_feature 1..357: note = Synthetic: RNF43-126 (VH DNA)
  source 1..357: mol_type = other DNA
gaggtgcagcttcaggagtcaggacctggccttgtgaaaccctcacagtcactctccctcacctgttctgtcactggtttctccatcaccagtagtcatagatggaactggatccggaagttcccaggaaataaactggagtggatgggatatataaacagtgcaggcagcactaactataacccgtctctcaaaagtcgaatctccattactagagacacatccaagaatcagttcttcctgcaggtgaactctgtaactactgaggacacagccacttattactgtacgagattaggactacagtatcgggactttgattactggggccaaggagtcatggtcacagtctcctca

SEQ ID NO: 62 - DNA, 321 bp, synthetic construct
  misc_feature 1..321: note = Synthetic: RNF43-126 (VL DNA)
  source 1..321: mol_type = other DNA
gacatccagatgacacagtctccagcttccctgtctgcatctctgggagaaactatctccatcgaatgtcgagcaagtgacgacatttacagtaatttagcgtggtatcagcagaagtcagggaaatctcctcagctcctgatctatgctgcaaataggttgaaagatggggtcccatcacggttcagtggcagtggatctggcacacagtattctctcaagatcagcggcatgcaaactgaagatgaaggggattatttctgtctacaggcttccaagtttccgctcacgttcggttctgggaccaagctggagatcaaa

SEQ ID NO: 63 - protein, 119 aa, synthetic construct
  REGION 1..119: note = Synthetic: RNF43-126 (VH prot)
EVQLQESGPGLVKPSQSLSLTCSVTGFSITSSHRWNWIRKFPGNKLEWMGYINSAGSTNYNPSLKSRISITRDTSKNQFFLQVNSVTTEDTATYYCTRLGLQYRDFDYWGQGVMVTVSS

SEQ ID NO: 64 - protein, 107 aa, synthetic construct
  REGION 1..107: note = Synthetic: RNF43-126(VL prot)
DIQMTQSPASLSASLGETISIECRASDDIYSNLAWYQQKSGKSPQLLIYAANRLKDGVPSRFSGSGSGTQYSLKISGMQTEDEGDYFCLQASKFPLTFGSGTKLEIK

SEQ ID NO: 65 - DNA, 339 bp, synthetic construct
  misc_feature 1..339: note = Synthetic: RNF43-128 (VH DNA)
  source 1..339: mol_type = other DNA
gaggtgcagcttcaggagtcaggacctggccttgtgaaaccctctcagtcactctccctcacctgttctgtcactggtttctccatcaccagtcattataaatggaactggatccggaagttcccaagaaataaactggagtggatgggatatatagacaattcaggcagcactaactacatcccgtctctcaaaagtcgaatctccattactagagacacatccaagaatcagttcttcctgcaggtgaactctgtaactactgaggacacagccacttattactgtgcgagggactgcctggactggggccaaggagtcatggtcacagtctcctca

SEQ ID NO: 66 - DNA, 336 bp, synthetic construct
  misc_feature 1..336: note = Synthetic: RNF43-128 (VL DNA)
  source 1..336: mol_type = other DNA
gatgttgtgctgacccagactccacccactttatcggctaccattggacaatcagtctccatctcttgcaggtcaagtcagagtctcttacatagtaatggaaacacctatttacattggttgatacagaggccaggccaatctccacagcttctaatttacttggtatccagactggaatctggggtccccaacaggttcagtggcagtgggtcaggaactgatttctcagtcaaaatcagtggagtagaggctgaggatttgggaatttattactgtgtgcaagatacccatactccgtggacgttcggtggaggcaccaagctggaattgaaa

SEQ ID NO: 67 - protein, 113 aa, synthetic construct
  REGION 1..113: note = Synthetic: RNF43-128 (VH prot)
EVQLQESGPGLVKPSQSLSLTCSVTGFSITSHYKWNWIRKFPRNKLEWMGYIDNSGSTNYIPSLKSRISITRDTSKNQFFLQVNSVTTEDTATYYCARDCLDWGQGVMVTVSS

SEQ ID NO: 68 - protein, 112 aa, synthetic construct
  REGION 1..112: note = Synthetic: RNF43-128(VL prot)
DVVLTQTPPTLSATIGQSVSISCRSSQSLLHSNGNTYLHWLIQRPGQSPQLLIYLVSRLESGVPNRFSGSGSGTDFSVKISGVEAEDLGIYYCVQDTHTPWTFGGGTKLELK

SEQ ID NO: 69 - DNA, 381 bp, synthetic construct
  misc_feature 1..381: note = Synthetic: RNF43-129 (VH DNA)
  source 1..381: mol_type = other DNA
gaggtgcagctggtggagtctgggggaggcttggtgcagcctggaaggtccatgaaactctcctgtgcagcctcaggattcactttcagtaactatgacatggcctgggtccgccaggctccaaagaagggtctggagtgggtcgcaagtattacttatgatgttagtagcacttactatcgagactccgtgaagggccgattcactatctccagagataatgcaaaaagcaccctatatttgcaaatggacagtctgaggtctgaggacacggccacttattactgtacaacattggatcgggcgggttattactatgatggttatggcttctattggtttgccaactggggccaaggcactctggtcactgtctcttca

SEQ ID NO: 70 - DNA, 321 bp, synthetic construct
  misc_feature 1..321: note = Synthetic: RNF43-129 (VL DNA)
  source 1..321: mol_type = other DNA
gacattcagatgacccagtctccatcctccatgtctgtgtctctgggagacacagtcactattacttgccgggcaagtcaggacgttaaaatttatgtaaactggttccagcagaaaccagggaaatctcccagacaaatgatttaccgtgcaacgaacttggcagatggggtctcatcaaggttcagcggcagtaggtctggatcagattatttattcaccatcagcagcctggagtctgaagatgtggcagactatcactgtctacagtatgatgagtatcctccaacgttcggttctgggaccaaggtggagatcaaa

SEQ ID NO: 71 - protein, 127 aa, synthetic construct
  REGION 1..127: note = Synthetic: RNF43-129 (VH prot)
EVQLVESGGGLVQPGRSMKLSCAASGFTFSNYDMAWVRQAPKKGLEWVASITYDVSSTYYRDSVKGRFTISRDNAKSTLYLQMDSLRSEDTATYYCTTLDRAGYYYDGYGFYWFANWGQGTLVTVSS

SEQ ID NO: 72 - protein, 107 aa, synthetic construct
  REGION 1..107: note = Synthetic: RNF43-129(VL prot)
DIQMTQSPSSMSVSLGDTVTITCRASQDVKIYVNWFQQKPGKSPRQMIYRATNLADGVSSRFSGSRSGSDYLFTISSLESEDVADYHCLQYDEYPPTFGSGTKVEIK

SEQ ID NO: 73 - DNA, 360 bp, synthetic construct
  misc_feature 1..360: note = Synthetic: RNF43-130 (VH DNA)
  source 1..360: mol_type = other DNA
caggttcagctggtccaatctggggctgaggtggtgaagcctgggtcctcagtgaaaatttcctgcaaggcttctggctacaccttcaccggtaactttattcactggataaaacagcagcctggcaatggccttgagtggattgggtggatttatcctggagatggtgatacaacatacaatcagaagttcagtgggaaggcaacactcactgcagacaagtcctccagcacagcctatatgcagctcagcaacctgacatctgaggactctgcagtctatttctgtgcaaagtggagggacctgagcagtgattggtttgcttactggggccaaggcactctggtcactgtctcttcc

SEQ ID NO: 74 - DNA, 339 bp, synthetic construct
  misc_feature 1..339: note = Synthetic: RNF43-130 (VL DNA)
  source 1..339: mol_type = other DNA
gacattgtgatgacccagtctccagtctccctgactgtgtcagaaggagagatggtcaccataaactgcaagtccagtcagagtcttttatccagtggaaaccaaaagaactacttggcctggtaccagcagaaaccagggcagtctccaagactactgatctactttacatccactagacaatcaggggtccctgatcgcttcataggcagtggatctgggacagacttcactctgaccatcagcgatgtgcaggctgaagacctggcagattattactgcctgcagcattacagctatccgaacacgtttggagctgggaccaagttggaactgaaa

SEQ ID NO: 75 - protein, 120 aa, synthetic construct
  REGION 1..120: note = Synthetic: RNF43-130 (VH prot)
QVQLVQSGAEVVKPGSSVKISCKASGYTFTGNFIHWIKQQPGNGLEWIGWIYPGDGDTTYNQKFSGKATLTADKSSSTAYMQLSNLTSEDSAVYFCAKWRDLSSDWFAYWGQGTLVTVSS

SEQ ID NO: 76 - protein, 113 aa, synthetic construct
  REGION 1..113: note = Synthetic: RNF43-130(VL prot)
DIVMTQSPVSLTVSEGEMVTINCKSSQSLLSSGNQKNYLAWYQQKPGQSPRLLIYFTSTRQSGVPDRFIGSGSGTDFTLTISDVQAEDLADYYCLQHYSYPNTFGAGTKLELK

SEQ ID NO: 77 - DNA, 360 bp, synthetic construct
  misc_feature 1..360: note = Synthetic: RNF43-136 (VH DNA)
  source 1..360: mol_type = other DNA
caggttcagctgcagcaatctggggctgaattagtgaagcctgggtcctcagtgaaaatttcctgcaaggcttctggctacaccttcaccagtaagtttatgcactggataaagcagcagcctggaagtggccttgagtggattgggtggatttatcctgaacatggtaatactaagtacaatcaaaagttcgatgggaaggcaacactcactgcagacaaatcctccagcacagcctatatgcaactcagcagcctgacatctggggactctgcagtctatttctgtgcaagacaaagcagccagggggcctatgctatggatgcctggggtcaaggaacttcagtcactgtctcctca

SEQ ID NO: 78 - DNA, 321 bp, synthetic construct
  misc_feature 1..321: note = Synthetic: RNF43-136 (VL DNA)
  source 1..321: mol_type = other DNA
gacatccaaatgacacagtctccagtttccctgtctgcatctcttggacaaactgtctccatcgaatgtctaacaagtgagggcattcccattgatttagcgtggtatcagcagaagtcagggaaatctcctcaactccttatctatgctgcaagtaggtcacaagacggggtcccatcacggttcagtggcagtggatctggcacacggtattctctcaagatcagcggcatgcaacctgaagatgaagcagattatttctgtcaacagacttacaagtatccattcacgttcggctcagggacgaggttggaaataaaa

SEQ ID NO: 79 - protein, 120 aa, synthetic construct
  REGION 1..120: note = Synthetic: RNF43-136 (VH prot)
QVQLQQSGAELVKPGSSVKISCKASGYTFTSKFMHWIKQQPGSGLEWIGWIYPEHGNTKYNQKFDGKATLTADKSSSTAYMQLSSLTSGDSAVYFCARQSSQGAYAMDAWGQGTSVTVSS

SEQ ID NO: 80 - protein, 107 aa, synthetic construct
  REGION 1..107: note = Synthetic: RNF43-136(VL prot)
DIQMTQSPVSLSASLGQTVSIECLTSEGIPIDLAWYQQKSGKSPQLLIYAASRSQDGVPSRFSGSGSGTRYSLKISGMQPEDEADYFCQQTYKYPFTFGSGTRLEIK

SEQ ID NO: 81 - DNA, 372 bp, synthetic construct
  misc_feature 1..372: note = Synthetic: RNF43-145 (VH DNA)
  source 1..372: mol_type = other DNA
gaggtgcagctgaaggaatcaggacctggtctggtgcagccctcacagaccctgtccctcacctgcactgtctctggattctcattaacggactacagtgtatactgggttcgccagcctccaggaaaaggtctggagtggatgggagtaatgtggagtggtggaaccacagcatataattcagctctcaaatcccgactgagcatcagcagggacacctccaagagccaagttttcttaaaaatgaacagtctgcaaactgaagacacagccatttactactgtaccagagggggcaaatatgatggtacttattactacggctggtactttgacttctggggcccaggaaccatggtcaccgtgtcctca

SEQ ID NO: 82 - DNA, 321 bp, synthetic construct
  misc_feature 1..321: note = Synthetic: RNF43-145 (VL DNA)
  source 1..321: mol_type = other DNA
gacgttgtgctgactcagtctccagccaccctgtctgtgactccaggagaaagaatcagtctctcctgccgggccagcgagagtgttgacacttatctatactggtatcagcaaaagccaaatgagtctccaaggcttctcattaagtatgcttcccagtccatctctggaataccctccaggttcagtggcagtggatctgggacagatttcactctcagtatcaatggagtagagcttgaagatttatcaatttattactgtcaacaaggtaacagcttgcccctcacgttcggctcagggacgaagttggaaataaaa

SEQ ID NO: 83 - protein, 124 aa, synthetic construct
  REGION 1..124: note = Synthetic: RNF43-145 (VH prot)
EVQLKESGPGLVQPSQTLSLTCTVSGFSLTDYSVYWVRQPPGKGLEWMGVMWSGGTTAYNSALKSRLSISRDTSKSQVFLKMNSLQTEDTAIYYCTRGGKYDGTYYYGWYFDFWGPGTMVTVSS

SEQ ID NO: 84 - protein, 107 aa, synthetic construct
  REGION 1..107: note = Synthetic: RNF43-145(VL prot)
DVVLTQSPATLSVTPGERISLSCRASESVDTYLYWYQQKPNESPRLLIKYASQSISGIPSRFSGSGSGTDFTLSINGVELEDLSIYYCQQGNSLPLTFGSGTKLEIK

SEQ ID NO: 85 - DNA, 360 bp, synthetic construct
  misc_feature 1..360: note = Synthetic: RNF43-152 (VH DNA)
  source 1..360: mol_type = other DNA
caggttactctgaaagagtctggccctgggatattgcagccctcccagaccctcagtctgacttgcaccttctctgggtcttcactgagcacttatggtatgggtgtgggctggattcgtcagccttcagggaagggtctggagtggctggcaaacatttggtgggatgatgataagtactacaatccatctctgaaaaaccggctcacaatctccaaggacacctccaacaaccaatcattcctcaagatcgccaatgtggacactgcagatactgccacatactactgtgctcggatagacggtacttataactactttgattattggggccaaggagtcatggtcacagtctcctca

SEQ ID NO: 86 - DNA, 336 bp, synthetic construct
  misc_feature 1..336: note = Synthetic: RNF43-152 (VL DNA)
  source 1..336: mol_type = other DNA
gatgttgtgttgacccagactccatccagactgtctgctaccattggacaatcggtctccatctcttgcaggtcaagtcagagtctcttagatagttatggaaacacctatttatcttggttcctacagaggccaggccaatctccacagcgtctaatttttttggtgtccaacctgggatctggggtccccaacagggtcagtggcagtgggtcaggaactgatttcacactcaaaatcagtggagtggaggctgaggatttgggagtttattactgcatgcaaactacccatgatcctctcacgttcggttctgggaccaagctggagatcaaa

SEQ ID NO: 87 - protein, 120 aa, synthetic construct
  REGION 1..120: note = Synthetic: RNF43-152 (VH prot)
QVTLKESGPGILQPSQTLSLTCTFSGSSLSTYGMGVGWIRQPSGKGLEWLANIWWDDDKYYNPSLKNRLTISKDTSNNQSFLKIANVDTADTATYYCARIDGTYNYFDYWGQGVMVTVSS

SEQ ID NO: 88 - protein, 112 aa, synthetic construct
  REGION 1..112: note = Synthetic: RNF43-152(VL prot)
DVVLTQTPSRLSATIGQSVSISCRSSQSLLDSYGNTYLSWFLQRPGQSPQRLIFLVSNLGSGVPNRVSGSGSGTDFTLKISGVEAEDLGVYYCMQTTHDPLTFGSGTKLEIK

SEQ ID NO: 89 - DNA, 372 bp, synthetic construct
  misc_feature 1..372: note = Synthetic: RNF43-156 (VH DNA)
  source 1..372: mol_type = other DNA
gaggtgcagctgaaggaatcaggacctggtctggtgcagccctcacagaccctgtccctcacctgcactgtctctggattctcattaacggactacagtgtatactgggttcgccagcctccaggaaaaggtctggagtggatgggagtaatgtggagaggtggaaccacagcatataattcagctctcaaatcccgactgagcatcagcagggacacctccaagagccaagttttcttaaaaatgaacagtctgcaaactgaagacacagccatttactactgtaccagagggggcaaatatgatggtacttattactacggctggtactttgacttctggggcccaggaaccatggtcaccgtgtcctca

SEQ ID NO: 90 - DNA, 321 bp, synthetic construct
  misc_feature 1..321: note = Synthetic: RNF43-156 (VL DNA)
  source 1..321: mol_type = other DNA
gacgttgtgctgactcagtctccagccaccctgtctgtgactccaggagaaagaatcagtctctcctgccgggccagcgagagtgttgacacttatctatactggtatcagcaaaaaccaaatgagtctccaaggcttctcattaagtatgcttcccagtccatctctggaataccctccaggttcagtggcagtggatctgggacagatttcactctcagtatcaatggagtagagcttgaagatttatcaatttattactgtcaacaaggtaacagcttgcccctcacgttcggctcagggacgaagttggaaataaaa

SEQ ID NO: 91 - protein, 124 aa, synthetic construct
  REGION 1..124: note = Synthetic: RNF43-156 (VH prot)
EVQLKESGPGLVQPSQTLSLTCTVSGFSLTDYSVYWVRQPPGKGLEWMGVMWRGGTTAYNSALKSRLSISRDTSKSQVFLKMNSLQTEDTAIYYCTRGGKYDGTYYYGWYFDFWGPGTMVTVSS

SEQ ID NO: 92 - protein, 107 aa, synthetic construct; identical to SEQ ID NO: 84
  REGION 1..107: note = Synthetic: RNF43-156(VL prot)

SEQ ID NO: 93 - DNA, 357 bp, synthetic construct
  misc_feature 1..357: note = Synthetic: RNF43-168 (VH DNA)
  source 1..357: mol_type = other DNA
caggtgcagctgaaggagtcaggacctggtctggtgcagccctcacagactttgtctctcacctgcactgtctctgggttctcactaaacaggtatcatgtaagctgggttcgccagcctccaggaaaaggtctggagtggatgggagtagtatggactggtggaagcacagcatataattcacttctcaaatcccgactgagcatcagcagggacatctccaagagccaagttttcttaaaaatgatcagtctgcaaactgaagacacagccacttactactgtgtcagtcaatactacgggtatacctcctactttgattactggggccaaggagtcatggtcacagtctcctca

SEQ ID NO: 94 - DNA, 321 bp, synthetic construct
  misc_feature 1..321: note = Synthetic: RNF43-168 (VL DNA)
  source 1..321: mol_type = other DNA
gacatccagatgacacagtctcctgcttccctgtctgcgtctccggaagaaattgtcacggtcacatgccaggcaagccaggacattgataattggttaacatggtatcagcagaaaccaggaaaatctcctcaactcctgatctatagtgcaaccagcttggcagacgggatcccatcaaggttcagcggcagtagatctggtacacagtattctcttaaaatcgacagactacaggttgaagatactggaatctattactgtctacagcgttatggtaatccgtggacgttcggtggaggcaccaagctggaattgaaa

SEQ ID NO: 95 - protein, 119 aa, synthetic construct
  REGION 1..119: note = Synthetic: RNF43-168 (VH prot)
QVQLKESGPGLVQPSQTLSLTCTVSGFSLNRYHVSWVRQPPGKGLEWMGVVWTGGSTAYNSLLKSRLSISRDISKSQVFLKMISLQTEDTATYYCVSQYYGYTSYFDYWGQGVMVTVSS

SEQ ID NO: 96 - protein, 107 aa, synthetic construct
  REGION 1..107: note = Synthetic: RNF43-168(VL prot)
DIQMTQSPASLSASPEEIVTVTCQASQDIDNWLTWYQQKPGKSPQLLIYSATSLADGIPSRFSGSRSGTQYSLKIDRLQVEDTGIYYCLQRYGNPWTFGGGTKLELK

SEQ ID NO: 97 - DNA, 351 bp, synthetic construct
  misc_feature 1..351: note = Synthetic: RNF43-170 (VH DNA)
  source 1..351: mol_type = other DNA
gaggtacaactggtggagtctgggggaggcttagtgccgcctggaaggtccctgaaactctcctgtgtagcctcaggattcactttcaatgactattacatggcctgggtccgccaggctccaacgaagggtctggagtgggtcgcatccattagttatgagggtcgtcgcacttactatggagactccgtgaagggccgattcactatctccagagataatgcaaaaagcaccctatatctgcaaatgaatagtctgaggtctgatgacacggccacttattattgtgcaacacatagcaactatatctttgattactggggccagggagtcatggtcacagtctcctca

SEQ ID NO: 98 - DNA, 321 bp, synthetic construct
  misc_feature 1..321: note = Synthetic: RNF43-170 (VL DNA)
  source 1..321: mol_type = other DNA
gatatccggatgacacagtctccagcttccctgtctgcatctctgggagaaactgtcaacatcgaatgtctagcaagtgaggacatttacagtgattttgcatggtttcagcagaagccagggcaatctcctctgctcctgatctataatggaaatagtttgcaaaatggggtcccttcacggtttagtggcagtggatctggcacacagtattctctaaaaatcaacagcctccaatctgaagatgtcgcgacttatttctgtcaacaatataacaattatcctccgacgttcggtggaggcaccaagctggaattgaaa

SEQ ID NO: 99 - protein, 117 aa, synthetic construct
  REGION 1..117: note = Synthetic: RNF43-170 (VH prot)
EVQLVESGGGLVPPGRSLKLSCVASGFTFNDYYMAWVRQAPTKGLEWVASISYEGRRTYYGDSVKGRFTISRDNAKSTLYLQMNSLRSDDTATYYCATHSNYIFDYWGQGVMVTVSS

SEQ ID NO: 100 - protein, 107 aa, synthetic construct
  REGION 1..107: note = Synthetic: RNF43-170(VL prot)
DIRMTQSPASLSASLGETVNIECLASEDIYSDFAWFQQKPGQSPLLLIYNGNSLQNGVPSRFSGSGSGTQYSLKINSLQSEDVATYFCQQYNNYPPTFGGGTKLELK

SEQ ID NO: 101 - DNA, 357 bp, synthetic construct
  misc_feature 1..357: note = Synthetic: RNF43-176 (VH DNA)
  source 1..357: mol_type = other DNA
caggtgcagctgaaggagtcaggacctggtctggtgcagccctcacagactttgtctctcacctgcactgtctctgggttctcactaaccagctatcatgtaagctgggttcgccagcctccaggaaaaggtctggagtggatgggagtaatatggactggtggaagcacagcatataattcacttctcaaatcccgactgagcatcagcagggacatctccaagagccaagttttcttaaaaatgaacagtctgcaaactgaagacacagccacttactactgtgccagtcaatactacgggtatacctcctattttgattactggggccaaggagtcatggtcacagtctcctca

SEQ ID NO: 102 - DNA, 321 bp, synthetic construct
  misc_feature 1..321: note = Synthetic: RNF43-176 (VL DNA)
  source 1..321: mol_type = other DNA
gacatccagatgacacagtctcctgtttccctgtctgcgtctccggaagaaattgtcacgatcacatgccaggcaagccaggacattgataattggttaacatggtatcagcagaaaccagggaaatctcctcaactcctgatctatagtgcaaccagcttggcagacgggatcccatcaaggttcagcggcagtagatctggtacacagtattctcttaagatcagcagactacagattgaagatactgggatctattactgtctacagcgttatggtaatccgtggacgttcggtggaggcaccaagctggaattgaaa

SEQ ID NO: 103 - protein, 119 aa, synthetic construct
  REGION 1..119: note = Synthetic: RNF43-176 (VH prot)
QVQLKESGPGLVQPSQTLSLTCTVSGFSLTSYHVSWVRQPPGKGLEWMGVIWTGGSTAYNSLLKSRLSISRDISKSQVFLKMNSLQTEDTATYYCASQYYGYTSYFDYWGQGVMVTVSS

SEQ ID NO: 104 - protein, 107 aa, synthetic construct
  REGION 1..107: note = Synthetic: RNF43-176(VL prot)
DIQMTQSPVSLSASPEEIVTITCQASQDIDNWLTWYQQKPGKSPQLLIYSATSLADGIPSRFSGSRSGTQYSLKISRLQIEDTGIYYCLQRYGNPWTFGGGTKLELK

SEQ ID NO: 105 - DNA, 348 bp, synthetic construct
  misc_feature 1..348: note = Synthetic: RNF43-177 (VH DNA)
  source 1..348: mol_type = other DNA
caggtcaagctgctgcagtctggggctgcactggtgaagcctggggactctgcgaagatgtcttgcaaagcttctggttatacattcactgactccattatacactgggtgaaacagagtcatggaaagagccttgagtggattggttatattaatccttacagtggtggtattcagtacaatgaaaagttcaagaacaaggccacattgactgtagacaaatccagcagcacagcctatgtggagtttagcagattgacatctgaagattctgcaatctattactgtgcaagagaaattcggggtatggatgcctggggtcaaggaacttcagtcactgtctcctca

SEQ ID NO: 106 - DNA, 321 bp, synthetic construct
  misc_feature 1..321: note = Synthetic: RNF43-177 (VL DNA)
  source 1..321: mol_type = other DNA
gacatcgtgctgactcagtctccagccaccctgtctgtgactccaggagagagtgtgagtctctcctgcagggccagtcagggtattagcactagtttacactggtatcagcaaaaatcaactgagtctccaaggcttctcatcaagtatgcttcccagtccgtctctggaatcccctccaggttcagtggcagtggatcagggacagatttcactctcagtatcaacagagtagaatctgaagatttttcagtttattactgtcaacagagttacaacttgccccgcacgtttggacctgggaccaagctggaactgaaa

SEQ ID NO: 107 - protein, 116 aa, synthetic construct
  REGION 1..116: note = Synthetic: RNF43-177 (VH prot)
QVKLLQSGAALVKPGDSAKMSCKASGYTFTDSIIHWVKQSHGKSLEWIGYINPYSGGIQYNEKFKNKATLTVDKSSSTAYVEFSRLTSEDSAIYYCAREIRGMDAWGQGTSVTVSS

SEQ ID NO: 108 - protein, 107 aa, synthetic construct
  REGION 1..107: note = Synthetic: RNF43-177(VL prot)
DIVLTQSPATLSVTPGESVSLSCRASQGISTSLHWYQQKSTESPRLLIKYASQSVSGIPSRFSGSGSGTDFTLSINRVESEDFSVYYCQQSYNLPRTFGPGTKLELK

SEQ ID NO: 109 - DNA, 351 bp, synthetic construct
  misc_feature 1..351: note = Synthetic: RNF43-179 (VH DNA)
  source 1..351: mol_type = other DNA
gaggtacaactggtggagtctgggggaggcttagtgcagcctggaaggtccctgaaactctcctgtgtagcctcaggattcactttcaatgactattacatggcctgggtccgccaggctccaaagaagggtctggagtgggtcgcatccattaattatgagggtagtcgcacttactatggagactccgtgaagggccgattcactatctccagagataatgcaaaaagcaccctatatctgcaaatgaatagtctgaggtctgaggacacggccacttattattgtgcaacacatagcaactatatctttgataattggggccagggagtcatggtcacagtctcctca

SEQ ID NO: 110 - DNA, 321 bp, synthetic construct
  misc_feature 1..321: note = Synthetic: RNF43-179 (VL DNA)
  source 1..321: mol_type = other DNA
gatatccggatgacacagtctccagcttccctgtctgcatctctgggagaaactgtcaacatcgaatgtctagcaagtgaggacatttacagtgattttgcatggtttcagcagaagccagggcaatctcctctgctcctgatctataatggaaataatttgcaaaatggggtcccttcacggtttagtggcagtggatctggcacacagtattctctaaaaatcaacagcctgcaatctgaagatgtcgcgacttatttctgtcaacaatataacaattatcctccgacgttcggtggaggcaccaaactggaattgaaa

SEQ ID NO: 111 - protein, 117 aa, synthetic construct
  REGION 1..117: note = Synthetic: RNF43-179 (VH prot)
EVQLVESGGGLVQPGRSLKLSCVASGFTFNDYYMAWVRQAPKKGLEWVASINYEGSRTYYGDSVKGRFTISRDNAKSTLYLQMNSLRSEDTATYYCATHSNYIFDNWGQGVMVTVSS

SEQ ID NO: 112 - protein, 107 aa, synthetic construct
  REGION 1..107: note = Synthetic: RNF43-179(VL prot)
DIRMTQSPASLSASLGETVNIECLASEDIYSDFAWFQQKPGQSPLLLIYNGNNLQNGVPSRFSGSGSGTQYSLKINSLQSEDVATYFCQQYNNYPPTFGGGTKLELK

SEQ ID NO: 113 - DNA, 354 bp, synthetic construct
  misc_feature 1..354: note = Synthetic: RNF43-180 (VH DNA)
  source 1..354: mol_type = other DNA
gaggcgcaattggtggagtctgggggaggcttagtgcggcctggaaggtccctgaaactcacctgtgcagcctcaggattcactttcagtcactatgacatggcctgggtccgccaggctccagcgaggggtctggagtgggtcgcatccattggtcctggtggtggtctcactcactatcgagactccgttaagggccgattcactgtctccagagatcatgcagaaagcagcctatacctgcaaatggacagtctgaggtctgaggacacggccacttattactgtgcaagacattacgggtggtcctactttgattactggggccaaggagtcatggtcacagtctcctca

SEQ ID NO: 114 - DNA, 321 bp, synthetic construct
  misc_feature 1..321: note = Synthetic: RNF43-180 (VL DNA)
  source 1..321: mol_type = other DNA
gacatccagatgacacagtctccgtcatttctgtctgcatctcttggagacagcatcaccatcacttgccatgccagtcagaacatcaagggttggttagcctggtatcaacaaaaatcggggaatgctcctgaactgttgatttatacggcttctggcctgcgatcaggggttccatcaagattcagtggcagcggatctggaacagattatattctcactatcagcaacctacagcctgaagattttgccacttattattgtcagcattatcaaagctttccgaacacgtttggagccgggaccaagctggaactgaga

SEQ ID NO: 115 - protein, 118 aa, synthetic construct
  REGION 1..118: note = Synthetic: RNF43-180 (VH prot)
EAQLVESGGGLVRPGRSLKLTCAASGFTFSHYDMAWVRQAPARGLEWVASIGPGGGLTHYRDSVKGRFTVSRDHAESSLYLQMDSLRSEDTATYYCARHYGWSYFDYWGQGVMVTVSS

SEQ ID NO: 116 - protein, 107 aa, synthetic construct
  REGION 1..107: note = Synthetic: RNF43-180(VL prot)
DIQMTQSPSFLSASLGDSITITCHASQNIKGWLAWYQQKSGNAPELLIYTASGLRSGVPSRFSGSGSGTDYILTISNLQPEDFATYYCQHYQSFPNTFGAGTKLELR

SEQ ID NO: 117 - DNA, 360 bp, synthetic construct
  misc_feature 1..360: note = Synthetic: RNF43-181 (VH DNA)
  source 1..360: mol_type = other DNA
cagatacagctgcagcaatctggggctgaactggtgaagcctgggtcctcagtgaaaatttcctgcaagtcttctgcctacaggttcaccagtaactttattcactggataagacagcagcctggcaatggccttgagtggattgggtggatttatcctggcgatggtgatgcagagaacaatcaaaagttcaatgggaaggtaatactcactgcagacaaatcgtccagcacagcctatatgcagctcagcagcctgacatctgaggactctgcggtctatttctgtgcaagaggagtgatgacgtcgttgtggtattttgacttttggggcccaggaaccatggtcaccgtgtcctca

SEQ ID NO: 118 - DNA, 321 bp, synthetic construct
  misc_feature 1..321: note = Synthetic: RNF43-181 (VL DNA)
  source 1..321: mol_type = other DNA
gatgtccagatgacccagtctccttataatcttgctgcctctcctggagaaagtgtttccatcaattgcaaggcaagtaagagcattagtaagtatttagcctggtatcaacagaaacctgggaaaacaaataagcttcttatctacgatgggtccactttgcaatctggaattccatcgaggttcagtggcagtggatctggtacagatttcactctcaccatcagaagcctggagcctgaagattttgggctctattactgtcaacagcatcatgaacagccacctacgttcggctcggggacgaagttggaaataaaa

SEQ ID NO: 119 - protein, 120 aa, synthetic construct
  REGION 1..120: note = Synthetic: RNF43-181 (VH prot)
QIQLQQSGAELVKPGSSVKISCKSSAYRFTSNFIHWIRQQPGNGLEWIGWIYPGDGDAENNQKFNGKVILTADKSSSTAYMQLSSLTSEDSAVYFCARGVMTSLWYFDFWGPGTMVTVSS

SEQ ID NO: 120 - protein, 107 aa, synthetic construct
  REGION 1..107: note = Synthetic: RNF43-181(VL prot)
DVQMTQSPYNLAASPGESVSINCKASKSISKYLAWYQQKPGKTNKLLIYDGSTLQSGIPSRFSGSGSGTDFTLTIRSLEPEDFGLYYCQQHHEQPPTFGSGTKLEIK

SEQ ID NO: 121 - DNA, 363 bp, synthetic construct
  misc_feature 1..363: note = Synthetic: RNF43-186 (VH DNA)
  source 1..363: mol_type = other DNA
caggtacagctacagcaatcagggactgaactggtgaagcctgcgtcctcagtgaaaatttcctgcaaggcttctggctacaccttcaccactaactatatgcactggataaggcagcagcctggaaatggccttgagtggattgggtggatttatcctggaaatgataatactaagtacaatcaaaagttcgatgggaaggcaacactcactgcagacaaatcctccagcacagcctatatgcagctcagcagcctgacatctgaggactctgcagtctatttctgtgcaagagggggattcgactacggagggtattggtttgcttactggggccagggcactccagtcactgtctcttca

SEQ ID NO: 122 - DNA, 321 bp, synthetic construct
  misc_feature 1..321: note = Synthetic: RNF43-186 (VL DNA)
  source 1..321: mol_type = other DNA
gatatccggatgacacagtctccagcttccctgtctgcatctctgggagaaactgtcaacatcgaatgtctatcaagtgaagacatttacagtgaattagcatggtatcagcagaagccagggaaatctcctcagctcctgatgtataatggaaatagcttgcaaaatggggtcccttcacggtttagtggcagtggatctggcacacaatattctctaaaaataaacagcctgcaatctgaagatgtcgcgacttatttctgtcaacaatatagaaattatccattcacgttcggctcagggacgaagttggaaataaaa

SEQ ID NO: 123 - protein, 121 aa, synthetic construct
  REGION 1..121: note = Synthetic: RNF43-186 (VH prot)
QVQLQQSGTELVKPASSVKISCKASGYTFTTNYMHWIRQQPGNGLEWIGWIYPGNDNTKYNQKFDGKATLTADKSSSTAYMQLSSLTSEDSAVYFCARGGFDYGGYWFAYWGQGTPVTVSS

SEQ ID NO: 124 - protein, 107 aa, synthetic construct
  REGION 1..107: note = Synthetic: RNF43-186(VL prot)
DIRMTQSPASLSASLGETVNIECLSSEDIYSELAWYQQKPGKSPQLLMYNGNSLQNGVPSRFSGSGSGTQYSLKINSLQSEDVATYFCQQYRNYPFTFGSGTKLEIK

SEQ ID NO: 125 - DNA, 360 bp, synthetic construct
  misc_feature 1..360: note = Synthetic: RNF43-187 (VH DNA)
  source 1..360: mol_type = other DNA
caggtccagctgcagcaatctggggctgaactggtgaagcctgggtcctcagtgaagatttcctgcaaggcttctggctacaccttcaccagtaactttatgcactggataaaacagcagcctggaaatggctttgagtggattgggtggatttatcctggagatggtgatacagactacaatcaaaagttcaatgggagggcaacactcactgcagacaaatcctccagcacagcctatatgcagctcagcagcctgacatctgaggactctgcagtctatttctgtgcaagatgggcgggccatcgtgaagattggtttccttactggggccaaggcactctggtcactgtctcttca

SEQ ID NO: 126 - DNA, 339 bp, synthetic construct
  misc_feature 1..339: note = Synthetic: RNF43-187 (VL DNA)
  source 1..339: mol_type = other DNA
gacattgtgatgacccaatctccatcctccctggctgtgtcagcaggagagacggtcactataaactgcaagtccagtcagagtcttttatccagtggaaaccaaaagaactacttggcctggtaccagcagaaaccagggcagtctcctaaactgctgatctacttggcatccactagggaatctggagtccctgatcgcttcataggcagtggatctgggacagacttcactctgaccatcagcagtgtgcaggctgaagatctggcagactattactgtcagcagcattacacctatccgaacacgtttggacctgggaccaagttggaactgaaa

SEQ ID NO: 127 - protein, 120 aa, synthetic construct
  REGION 1..120: note = Synthetic: RNF43-187 (VH prot)
QVQLQQSGAELVKPGSSVKISCKASGYTFTSNFMHWIKQQPGNGFEWIGWIYPGDGDTDYNQKFNGRATLTADKSSSTAYMQLSSLTSEDSAVYFCARWAGHREDWFPYWGQGTLVTVSS

SEQ ID NO: 128 - protein, 113 aa, synthetic construct
  REGION 1..113: note = Synthetic: RNF43-187(VL prot)
DIVMTQSPSSLAVSAGETVTINCKSSQSLLSSGNQKNYLAWYQQKPGQSPKLLIYLASTRESGVPDRFIGSGSGTDFTLTISSVQAEDLADYYCQQHYTYPNTFGPGTKLELK

SEQ ID NO: 129 - DNA, 360 bp, synthetic construct
  misc_feature 1..360: note = Synthetic: RNF43-196 (VH DNA)
  source 1..360: mol_type = other DNA
caggtacagctgcagcaatctggggctgaactggtgaagcctgggtcctcagtgaaaatttcctgcaaggcttctggcttcaccttcaccagtaactttatgcactggataaaacagcagcctggaaatggccttgagtggattgggtggatttatcctggagatggtgatacagactacaatcaaaagttcaatgggaaggcaactctcactgcagacaaatcctccagcacagcctatatggagctcagcagcctgacatctgaggactctgcagtctatttctgtgcaagagcgtactacggtagctactggtactttgacttctggggcccaggaaccaaggtcaccgtgtcctca

SEQ ID NO: 130 - DNA, 321 bp, synthetic construct
  misc_feature 1..321: note = Synthetic: RNF43-196 (VL DNA)
  source 1..321: mol_type = other DNA
gatgtccagatgacccagtctccacataatcttgctgcctctcctggagaaagtgtttccatcaattgcaaggcaagtaagagcattagcaagtatttagcctggtatcagcagaaacctgggaaagtaaaaaagcttcttatctacggtgggtcaactttgcaatctggaattccatcgaggttcagtggcagtggatctggtacagatttcactctcaccatcagcagcctggagcctgaagattttggactctattactgtcaacagcataatgaatacccaccgacgttcggtggaggcaccaggctggaattgcaa

SEQ ID NO: 131 - protein, 120 aa, synthetic construct
  REGION 1..120: note = Synthetic: RNF43-196 (VH prot)
QVQLQQSGAELVKPGSSVKISCKASGFTFTSNFMHWIKQQPGNGLEWIGWIYPGDGDTDYNQKFNGKATLTADKSSSTAYMELSSLTSEDSAVYFCARAYYGSYWYFDFWGPGTKVTVSS

SEQ ID NO: 132 - protein, 107 aa, synthetic construct
  REGION 1..107: note = Synthetic: RNF43-196(VL prot)
DVQMTQSPHNLAASPGESVSINCKASKSISKYLAWYQQKPGKVKKLLIYGGSTLQSGIPSRFSGSGSGTDFTLTISSLEPEDFGLYYCQQHNEYPPTFGGGTRLELQ

SEQ ID NO: 133 - DNA, 357 bp, synthetic construct
  misc_feature 1..357: note = Synthetic: RNF43-200 (VH DNA)
  source 1..357: mol_type = other DNA
caggtgcagctgaaggagtcaggacctggtctggtgcagccctcacagactttgtctctcacctgcactgtctctgggttctcactaaccagctatcatataagctgggttcgccagcctccaggaaaaggtctggagtggatgggagtaatatggactggtggaagcacagcatataattcacttctcaaatcccgactgagcatcagcagggacatctccaagagccaagtcttcttaaaaatgaacagtctgcaagctgaagacacagccacttactactgtgccggatcatactacgggtatacctcctactttgattactggggccaaggagtcatggtcacagtctcctca

SEQ ID NO: 134 - DNA, 321 bp, synthetic construct
  misc_feature 1..321: note = Synthetic: RNF43-200 (VL DNA)
  source 1..321: mol_type = other DNA
gacatccagatgacacagtctcctgcttccctgtctgcgtctccggaagaaattgtcacgatcacatgccaggcaagccaggacattgataattggttagcatggtatcagcagaaaccagggaaatctcctcaactcctgatctatagtgcaaccaggttggcagacgggatcccatcaaggttcagcggcagtagatctggtatacagtattctcttaagatcaacagactgcaggttgaagatactggaatctattactgtctacagcgttttggtaatccttggacgttcggtggaggcaccaagctggaattgaaa

SEQ ID NO: 135 - protein, 119 aa, synthetic construct
  REGION 1..119: note = Synthetic: RNF43-200 (VH prot)
QVQLKESGPGLVQPSQTLSLTCTVSGFSLTSYHISWVRQPPGKGLEWMGVIWTGGSTAYNSLLKSRLSISRDISKSQVFLKMNSLQAEDTATYYCAGSYYGYTSYFDYWGQGVMVTVSS

SEQ ID NO: 136 - protein, 107 aa, synthetic construct
  REGION 1..107: note = Synthetic: RNF43-200(VL prot)
DIQMTQSPASLSASPEEIVTITCQASQDIDNWLAWYQQKPGKSPQLLIYSATRLADGIPSRFSGSRSGIQYSLKINRLQVEDTGIYYCLQRFGNPWTFGGGTKLELK

SEQ ID NO: 137 - DNA, 360 bp, synthetic construct
  misc_feature 1..360: note = Synthetic: RNF43-201 (VH DNA)
  source 1..360: mol_type = other DNA
caggttactctgaaagagtatggccctgggatattgcagccctcccagaccctcagtctgacttgcactttctctgggttttcactgaggacttatggtatgggtgtgggctggattcgtcagccttcagggaagggtctggagtggctgacaaatatttggtgggatgatgataagtactacaatccatctctgaaaaaccggctcacaatctccaaggacacctccaacaaccaagcattcctcaagatcaccaatgtggacactgaggatagtgccacatactattgtgctcggatagacggtacttttaactactttgattattggggccaaggagtcatggtcacagtctcctca

SEQ ID NO: 138 - DNA, 336 bp, synthetic construct
  misc_feature 1..336: note = Synthetic: RNF43-201 (VL DNA)
  source 1..336: mol_type = other DNA
gatgttgtgctgacccagactccttccagattgtctgctaccattggacaatcggtctccatctcttgcaggtcaagtcagagtctcttagatagtgatggaaacacctatttatattggttcctacagaggccaggccaatctccacagcgtctaatttatttggtatccaacctgggatctggggtccccaacagggtcagtggcagtgggtcaggaacagatttcacactcaaaatcagtggagtggaggctgaggatttgggaatttattactgcttacaagctacccatgctcctctcacgttcggttctgggaccaagctggagatcaaa

SEQ ID NO: 139 - protein, 120 aa, synthetic construct
  REGION 1..120: note = Synthetic: RNF43-201 (VH prot)
QVTLKEYGPGILQPSQTLSLTCTFSGFSLRTYGMGVGWIRQPSGKGLEWLTNIWWDDDKYYNPSLKNRLTISKDTSNNQAFLKITNVDTEDSATYYCARIDGTFNYFDYWGQGVMVTVSS

SEQ ID NO: 140 - protein, 112 aa, synthetic construct
  REGION 1..112: note = Synthetic: RNF43-201(VL prot)
DVVLTQTPSRLSATIGQSVSISCRSSQSLLDSDGNTYLYWFLQRPGQSPQRLIYLVSNLGSGVPNRVSGSGSGTDFTLKISGVEAEDLGIYYCLQATHAPLTFGSGTKLEIK

SEQ ID NO: 141 - DNA, 357 bp, synthetic construct
  misc_feature 1..357: note = Synthetic: RNF43-206 (VH DNA)
  source 1..357: mol_type = other DNA
caggtgcagctgaaggagtcaggacctggtctggtgcagccctcacagactttgtctctcacctgcactgtctctgggttctcactaaccagttttcatataagctgggttcgccagcctccaggaaaaggtctggagtggatgggaataatatggactggtggaagcacagcatataattcacttctcaaatcccgactgagcatcagcagggacatctccaagagccaagtcttcttaaaaatgaacagtctgcaaactgaagacacagccacttactactgtgccggatcatatcacgggtatacctcctactttgattactggggccaaggagtcatggtcacagtctcctca

SEQ ID NO: 142 - DNA, 321 bp, synthetic construct
  misc_feature 1..321: note = Synthetic: RNF43-206 (VL DNA)
  source 1..321: mol_type = other DNA
gacatccagatgactcagtctcctgcttccctgtctgcgtctccggaagaaattgtcacgatcacatgccaggcaagccaggacattgataattggttagcatggtatcagcagcaaccagggaaatctcctcaactcctgatctatagtgcaactagcttggcagacgggatcccatcaaggttcagcggcagtagatctggtacacagtattctcttaagatcaacagactacaggttgaagatactggaatctattactgtctacagcgttatagtaatccttggacgttcggtggaggcaccaagctggaattgaaa

SEQ ID NO: 143 - protein, 119 aa, synthetic construct
  REGION 1..119: note = Synthetic: RNF43-206 (VH prot)
QVQLKESGPGLVQPSQTLSLTCTVSGFSLTSFHISWVRQPPGKGLEWMGIIWTGGSTAYNSLLKSRLSISRDISKSQVFLKMNSLQTEDTATYYCAGSYHGYTSYFDYWGQGVMVTVSS

SEQ ID NO: 144 - protein, 107 aa, synthetic construct
  REGION 1..107: note = Synthetic: RNF43-206(VL prot)
DIQMTQSPASLSASPEEIVTITCQASQDIDNWLAWYQQQPGKSPQLLIYSATSLADGIPSRFSGSRSGTQYSLKINRLQVEDTGIYYCLQRYSNPWTFGGGTKLELK

SEQ ID NO: 145 - DNA, 363 bp, synthetic construct
  misc_feature 1..363: note = Synthetic: RNF43-210 (VH DNA)
  source 1..363: mol_type = other DNA
cagatccagttggtacagtctggacctgagctgaagaagcctgaagagtcagtgaagatctcctgcaaggcttctgggtatatcttcacagactatttaatacactgggtgaagcaggctccaggagagggcttgaagtggatgggctggatcaacacctatactgggaagccaacatatgctgatgacttcaaaggacggtttgtcttttctttggaagcctctgccagcactgcaattttgcagatcagcaacctcaaaaatgaggacacggctacatatttctgtgctctgggccccattactacagtcgtctatgtcatggatgcctggggtcaaggaacttcagtcactgtctcctca

SEQ ID NO: 146 - DNA, 321 bp, synthetic construct
  misc_feature 1..321: note = Synthetic: RNF43-210 (VL DNA)
  source 1..321: mol_type = other DNA
gacatccagatgacccagtctccttcattcctgtctgcatctgtgggagacagagtcactatcaactgcaaagcaagtcagaatattaacaagtacttaaactggtatcagcaaaagcttggagaagctcccaaacgcctgatatataatacaaacaatttgcaaacagacatcccatcaaggttcagtggcagtggatctggtgcagattacacactcaccatcaacagcctgcagcctgaagattttgccacatatttctgcttgcagcataatagttttccgtacacgtttggagctgggaccaaactggaactgaaa

SEQ ID NO: 147 - protein, 121 aa, synthetic construct
  REGION 1..121: note = Synthetic: RNF43-210 (VH prot)
QIQLVQSGPELKKPEESVKISCKASGYIFTDYLIHWVKQAPGEGLKWMGWINTYTGKPTYADDFKGRFVFSLEASASTAILQISNLKNEDTATYFCALGPITTVVYVMDAWGQGTSVTVSS

SEQ ID NO: 148 - protein, 107 aa, synthetic construct
  REGION 1..107: note = Synthetic: RNF43-210(VL prot)
DIQMTQSPSFLSASVGDRVTINCKASQNINKYLNWYQQKLGEAPKRLIYNTNNLQTDIPSRFSGSGSGADYTLTINSLQPEDFATYFCLQHNSFPYTFGAGTKLELK

SEQ ID NO: 149 - DNA, 357 bp, synthetic construct
  misc_feature 1..357: note = Synthetic: RNF43-213 (VH DNA)
  source 1..357: mol_type = other DNA
caggtgcagctgaaggagtcaggacctggtctggtgcagccctcacagactttgtctctcacctgcactgtctctgggttctcactaaccaggtatcatgtaagctgggttcgccagcctccaggaaaaggtctggagtggatgggagtaatatggactggtggaagcacagcatataattcacttctcaaatcccgactgagcatcagcagggacatctccaagagccaagttttcttaaaaatgaacagtctgcaaactgaagacacagccacttactactgtgccagtcaatactacgggtatacctcctactttgattactggggccaaggagtcatggtcacagtctccgca

SEQ ID NO: 150 - DNA, 321 bp, synthetic construct
  misc_feature 1..321: note = Synthetic: RNF43-213 (VL DNA)
  source 1..321: mol_type = other DNA
gacatccagatgacacagtctcctgcttccctgtctgcgtctccggaagaaattgtcacgatcacatgccaggcaagccaggacattgctaattggttagcatggtatcagcagaaaccagggaaatctcctcaactcctgatctatagtgcaaccaacttggcagacgggatcccatcaaggttcagcggcagtagatctggtacacagtattctcttaagatcagcagactacaggttgaagatactggagtctattactgtctacagcgttatggtaatccgtggacgttcggtggaggcaccaacctggaattgaaa

SEQ ID NO: 151 - protein, 119 aa, synthetic construct
  REGION 1..119: note = Synthetic: RNF43-213 (VH prot)
QVQLKESGPGLVQPSQTLSLTCTVSGFSLTRYHVSWVRQPPGKGLEWMGVIWTGGSTAYNSLLKSRLSISRDISKSQVFLKMNSLQTEDTATYYCASQYYGYTSYFDYWGQGVMVTVSA

SEQ ID NO: 152 - protein, 107 aa, synthetic construct
  REGION 1..107: note = Synthetic: RNF43-213(VL prot)
DIQMTQSPASLSASPEEIVTITCQASQDIANWLAWYQQKPGKSPQLLIYSATNLADGIPSRFSGSRSGTQYSLKISRLQVEDTGVYYCLQRYGNPWTFGGGTNLELK

SEQ ID NO: 153 - DNA, 357 bp, synthetic construct
  misc_feature 1..357: note = Synthetic: RNF43-217 (VH DNA)
  source 1..357: mol_type = other DNA
gaggtgcagcttcaggagtcaggacctggccttgtgaaaccctcacagtcactctccctcacctgttctgtcactggtttctccatcaccagtagtcatagatggaactggatccggaagttcccaggaaataaactggagtggatgggatatataaacagtgcaggcagcactaactataacccgtctctcaaaagtcgagtctccattactagagacgcatccaagaatcagttcttcctgcaggtgaactctgtaactactgaggacacagccacttattactgtacgagattaggactacagtatcgggactttgattactggggccagggagtcatggtcacagtctcctca

SEQ ID NO: 154 - DNA, 321 bp, synthetic construct
  misc_feature 1..321: note = Synthetic: RNF43-217 (VL DNA)
  source 1..321: mol_type = other DNA
gacatccagatgacacagtctccagcttccctgtctgcatctctgggagaaactatctccatcgaatgtcgagcaagtgacgacatttacagtaatttagcgtggtatcagcagaagtcagggaaatctcctcagctcctgatctatgctgcaaataggttgaaagatggggtcccatcacggttcagtggcagtgggtctggcacacagtattctctcaagatcagcggcatggaaactgaagatgaaggggattatttctgtctacaggcttccaagtttccgctcacgttcggttctgggaccaagctggagatcaaa

SEQ ID NO: 155 - protein, 119 aa, synthetic construct
  REGION 1..119: note = Synthetic: RNF43-217 (VH prot)
EVQLQESGPGLVKPSQSLSLTCSVTGFSITSSHRWNWIRKFPGNKLEWMGYINSAGSTNYNPSLKSRVSITRDASKNQFFLQVNSVTTEDTATYYCTRLGLQYRDFDYWGQGVMVTVSS

SEQ ID NO: 156 - protein, 107 aa, synthetic construct
  REGION 1..107: note = Synthetic: RNF43-217(VL prot)
DIQMTQSPASLSASLGETISIECRASDDIYSNLAWYQQKSGKSPQLLIYAANRLKDGVPSRFSGSGSGTQYSLKISGMETEDEGDYFCLQASKFPLTFGSGTKLEIK

SEQ ID NO: 157 - DNA, 345 bp, synthetic construct
  misc_feature 1..345: note = Synthetic: RNF43-221 (VH DNA)
  source 1..345: mol_type = other DNA
gaggtgcagctggtggagtctggcggaggcttagtgcagcctggaaggtccctaaaactctcctgtacagcctcaggattcactctcagtgactatgccatggcctgggtccgccaggctccagagaaaggtctggaatgggtcgcaaccataagttatgatggtagtagaacttactatggaggctccgtgaagggccgattcactatctccagagataatgcagaaaatatcctatacctgcaaatggacagtctgaggtctgcggacacggccacttattattgtgcaaactcagcaactccccattactggggccaaggagtcatggtcacagtctcctca

SEQ ID NO: 158 - DNA, 336 bp, synthetic construct
  misc_feature 1..336: note = Synthetic: RNF43-221 (VL DNA)
  source 1..336: mol_type = other DNA
gatgttgtgatgacccagactccagcgtctttgtcgcttgccattggacaaccagcctccatctcttgcaagtcaagccagagcctcttagatactagtcgaaagacatttttgaattggatattacagaggcccggccagtctccaaagcgcctaatctatcaggtttccaaactgtactcagaggttcctgataggttcagtggcagtggatcagagacagagtttactcttaaaatcagcagagtggaggctgaggatttgggagtgtattactgctggcaaggtacacattttccggacacgtttggagctgggaccaagctggaactgaaa

SEQ ID NO: 159 - protein, 115 aa, synthetic construct
  REGION 1..115: note = Synthetic: RNF43-221 (VH prot)
EVQLVESGGGLVQPGRSLKLSCTASGFTLSDYAMAWVRQAPEKGLEWVATISYDGSRTYYGGSVKGRFTISRDNAENILYLQMDSLRSADTATYYCANSATPHYWGQGVMVTVSS

SEQ ID NO: 160 - protein, 112 aa, synthetic construct
  REGION 1..112: note = Synthetic: RNF43-221(VL prot)
DVVMTQTPASLSLAIGQPASISCKSSQSLLDTSRKTFLNWILQRPGQSPKRLIYQVSKLYSEVPDRFSGSGSETEFTLKISRVEAEDLGVYYCWQGTHFPDTFGAGTKLELK

SEQ ID NO: 161 - DNA, 372 bp, synthetic construct
  misc_feature 1..372: note = Synthetic: RNF43-224 (VH DNA)
  source 1..372: mol_type = other DNA
gaggtgcagctgaaggaatcaggacctggtctggtgcagccctcacagaccctgtccctcacctgcactgtctctggattctcattgtcggactacagtgtacactgggttcgccagcctccaggaaaaggtctggagtggatgggagttatgtggagtggtggaaccacagcatataattcagctctcaaatcccgactgagcatcagcagggacacctccaagaaccaagttctcttagaaatgaacagtctgcaaactgaagacacagccatttactactgtaccagagggggcaaatatgatggtacttattactacggctggtactttgacttctggggcccaggaaccatggtcaccgtgtcctca

SEQ ID NO: 162 - DNA, 321 bp, synthetic construct
  misc_feature 1..321: note = Synthetic: RNF43-224 (VL DNA)
  source 1..321: mol_type = other DNA
gacgttgtgctgactcagtctccagccaccctgtctgtgactccaggagaaagaatcagtctctcctgtcgggccagcgagagtgttgacacttatctatcctggtatcagcaaaaaccaaatgagtctccaaggcttctcattaagtatgcttcccagtccatctctggaattccctccaggttcagtggcagtggatctgggacagatttcactctcagtatcaatggagtagagcttgaagatttatcaatttattactgtcaacagggtaacagcttgcccctcacgttcggctcagggacgaagttggaaataaaa

SEQ ID NO: 163 - protein, 124 aa, synthetic construct
  REGION 1..124: note = Synthetic: RNF43-224 (VH prot)
EVQLKESGPGLVQPSQTLSLTCTVSGFSLSDYSVHWVRQPPGKGLEWMGVMWSGGTTAYNSALKSRLSISRDTSKNQVLLEMNSLQTEDTAIYYCTRGGKYDGTYYYGWYFDFWGPGTMVTVSS

SEQ ID NO: 164 - protein, 107 aa, synthetic construct
  REGION 1..107: note = Synthetic: RNF43-224(VL prot)
DVVLTQSPATLSVTPGERISLSCRASESVDTYLSWYQQKPNESPRLLIKYASQSISGIPSRFSGSGSGTDFTLSINGVELEDLSIYYCQQGNSLPLTFGSGTKLEIK

SEQ ID NO: 165 - DNA, 360 bp, synthetic construct
  misc_feature 1..360: note = Synthetic: RNF43-25 (VH DNA)
  source 1..360: mol_type = other DNA
caggtcaagctgctgcagtctggggctgcactggtgaagcctggggactctgtgaagatgtcttgcaaagcttctggttatacattcactgactacattatacattgggtgaagcagagtcatggaaggagccttgagtggattgcttatattaatccttacagtggtagtactaactacaatgaaaagttcaagagcaaggccacattgactgtagacagatccaacaacacagcctatatggagtttagcagattgacatctgaggattctgcaatctactactgtgcaagaaagaactacggaggttatggagttatggatgcctggggtcaaggagcttcagtcactgtctcctca

SEQ ID NO: 166 - DNA, 318 bp, synthetic construct
  misc_feature 1..318: note = Synthetic: RNF43-25 (VL DNA)
  source 1..318: mol_type = other DNA
gacatccagatgacccagtctccttcactcctgtctgcatctgtgggagacagagtcactctcagctgcaaagcaagtcagaatatttataagaacttagcctggcatcagcaaaagcttggagaagctcccaaactcctgatttattatgcaaacaccttgcaaacgggcatcccatcaaggttcagtggcagtggatttggtacagatttcacactcacaatcagcagcctgcagcctgaagatgttgccacatatttctgccagcagtcttctagcgggtggacgttcggtggaggcaccaagctggaattgaaa

SEQ ID NO: 167 - protein, 120 aa, synthetic construct
  REGION 1..120: note = Synthetic: RNF43-25 (VH prot)
QVKLLQSGAALVKPGDSVKMSCKASGYTFTDYIIHWVKQSHGRSLEWIAYINPYSGSTNYNEKFKSKATLTVDRSNNTAYMEFSRLTSEDSAIYYCARKNYGGYGVMDAWGQGASVTVSS

SEQ ID NO: 168 - protein, 106 aa, synthetic construct
  REGION 1..106: note = Synthetic: RNF43-25 (VL prot)
DIQMTQSPSLLSASVGDRVTLSCKASQNIYKNLAWHQQKLGEAPKLLIYYANTLQTGIPSRFSGSGFGTDFTLTISSLQPEDVATYFCQQSSSGWTFGGGTKLELK

SEQ ID NO: 169 - DNA, 363 bp, synthetic construct
  misc_feature 1..363: note = Synthetic: RNF43-31 (VH DNA)
  source 1..363: mol_type = other DNA
gcggtacagctggtggagtctgggggaggctcagtgcagcctggaaggtccatgcggctctcctgcgcagcctcaggattcactttcagtaactatgacatgacctgggtccgccaggctccaacgaagggtctggagtgggtcgcatccattacttatgatggtggtagtacttactctcgagactccgtgaagggccgattcactatctccagagataatgcaaaaagcaccctatacctgcaaatggacagtctgaggtctgaggacacggccacttattactgtacaacagatcggggtagatacctaccctactactttgattactggggccaaggagtcatggtcacagtctccgca

SEQ ID NO: 170 - DNA, 333 bp, synthetic construct
  misc_feature 1..333: note = Synthetic: RNF43-31 (VL DNA)
  source 1..333: mol_type = other DNA
gatgttgtgctgacacaaactccagtttccctgtctgtcacacttggagatcaagcttctatatcttgcaggtctagtcagagcctggaatatagtgatggatatagttatttggaatggtacctacagaagccaggccagtctccacagctcctcatctatgaagtttccagccgattttctggggtcccagacaggttcattggcagtgggtcagggacagatttcaccctcaagatcagcagagtagagcctgaggacttgggagtttattactgcttccaagctatacatgatcccacgtttggagctgggaccaagctggaactgaaa

SEQ ID NO: 171 - protein, 121 aa, synthetic construct
  REGION 1..121: note = Synthetic: RNF43-31 (VH prot)
AVQLVESGGGSVQPGRSMRLSCAASGFTFSNYDMTWVRQAPTKGLEWVASITYDGGSTYSRDSVKGRFTISRDNAKSTLYLQMDSLRSEDTATYYCTTDRGRYLPYYFDYWGQGVMVTVSA

SEQ ID NO: 172 - protein, 111 aa, synthetic construct
  REGION 1..111: note = Synthetic: RNF43-31(VL prot)
DVVLTQTPVSLSVTLGDQASISCRSSQSLEYSDGYSYLEWYLQKPGQSPQLLIYEVSSRFSGVPDRFIGSGSGTDFTLKISRVEPEDLGVYYCFQAIHDPTFGAGTKLELK

SEQ ID NO: 173 - DNA, 342 bp, synthetic construct
  misc_feature 1..342: note = Synthetic: RNF43-33 (VH DNA)
  source 1..342: mol_type = other DNA
caggagcagctgaaggagtcaggacctggtctggtgcagccctcacagaccctgtccctcacctgcactgtctctgggttctcattaaccagctataatgtacactgggttcgacagcctccaagaaagggtctggagtggatgggaagaatgagatataatggagacacatcatataattcagctctcaaatccagactgagcattagcagggacacctccaagaaccaagttttcttaaaaatgaacagtctgcaaactgatgacacaggcacttactactgtgccagagggcccctctgggattactggggccaaggagtcatggtcacagtctcctca

SEQ ID NO: 174 - DNA, 336 bp, synthetic construct
  misc_feature 1..336: note = Synthetic: RNF43-33 (VL DNA)
  source 1..336: mol_type = other DNA
gatgttgtgctgacccagactccacccactttatcggctaccattggacaatcagtctccgtctcttgcaggtcaagtcagagtctcttacatagtaatggaaacacctatttaaattggttgctacagaggccaggccaacctccacaacttctaatttatttggtatccagactggaatctggggtccccaacaggttcagtggcagtgggtcaggaactgatttcacactcaaaatcagtggagtagaggctgaggatttgggagtttattactgcgtgcaaagtacccatgctccgtacacgtttggagctgggaccaagctggaactgaaa

SEQ ID NO: 175 - protein, 114 aa, synthetic construct
  REGION 1..114: note = Synthetic: RNF43-33 (VH prot)
QEQLKESGPGLVQPSQTLSLTCTVSGFSLTSYNVHWVRQPPRKGLEWMGRMRYNGDTSYNSALKSRLSISRDTSKNQVFLKMNSLQTDDTGTYYCARGPLWDYWGQGVMVTVSS

SEQ ID NO: 176 - protein, 112 aa, synthetic construct
  REGION 1..112: note = Synthetic: RNF43-33(VL prot)
DVVLTQTPPTLSATIGQSVSVSCRSSQSLLHSNGNTYLNWLLQRPGQPPQLLIYLVSRLESGVPNRFSGSGSGTDFTLKISGVEAEDLGVYYCVQSTHAPYTFGAGTKLELK

SEQ ID NO: 177 - DNA, 357 bp, synthetic construct
  misc_feature 1..357: note = Synthetic: RNF43-35 (VH DNA)
  source 1..357: mol_type = other DNA
caggtacagttgaaggagtcaggacctggtctggtgcagccctcgcagaccctgtccctcacctgcactgtctctgggttctcactaaccacctacagtgtccactgggttcgccaacattcaggaaagaatctggaatggatgggaagaatgtggactgctggagacacatcatataattcagcgttcacatcccgactgaacatcttcagggacacctccaagagccaagttttcttaaaaatgaacagtctgcaaactgaggacacaggcacttactactgtgccagatcctcctatacttcgggttacccttttgattcctggggccaaggagtcatggtcacagtctcctca

SEQ ID NO: 178 - DNA, 315 bp, synthetic construct
  misc_feature 1..315: note = Synthetic: RNF43-35 (VL DNA)
  source 1..315: mol_type = other DNA
gacactgtactgacccagtctcctgctttggctgtgtctccaggagagagggttaccatctcctgtagggccagtgaaagtgtcagtaaacttatgcactggtaccaacagagaccaggacaacaaccccaactcctcatctatctaacatcacacctagcatctggggtccctgccaggttcagtggcagtgggtctgggacagacttcaccctcaccattgatcctgtggaggctgatgacactgcaacctattactgtcagcagagtcggaatgatcccacgtttggagctgggaccaagctggaactgaaa

SEQ ID NO: 179 - protein, 119 aa, synthetic construct
  REGION 1..119: note = Synthetic: RNF43-35 (VH prot)
QVQLKESGPGLVQPSQTLSLTCTVSGFSLTTYSVHWVRQHSGKNLEWMGRMWTAGDTSYNSAFTSRLNIFRDTSKSQVFLKMNSLQTEDTGTYYCARSSYTSGYPFDSWGQGVMVTVSS

SEQ ID NO: 180 - protein, 105 aa, synthetic construct
  REGION 1..105: note = Synthetic: RNF43-35(VL prot)
DTVLTQSPALAVSPGERVTISCRASESVSKLMHWYQQRPGQQPQLLIYLTSHLASGVPARFSGSGSGTDFTLTIDPVEADDTATYYCQQSRNDPTFGAGTKLELK

SEQ ID NO: 181 - DNA, 369 bp, synthetic construct
  misc_feature 1..369: note = Synthetic: RNF43-38 (VH DNA)
  source 1..369: mol_type = other DNA
gaagtacagcttgtggagtctggaggaggcttggtgcaacctgggacttctctgaaaatctcttgtgtagcctcgggattcactttcagtgactactggatgagctgggttcgccagactcctggaaagaccatggagtggattggagatattaaatctgatggcagttacacaaactatgcaccatccctaaagaatcgattcacaatctccagagacaatgccaagagcaccctgtacctgcagatgaacaatgtgagatctgaagacacagccacttattactgtactagagatgcatataggtacaactacgtccactatggtttggatgcctggggccaaggagcttcagtcactgtctcctca

SEQ ID NO: 182 - DNA, 333 bp, synthetic construct
  misc_feature 1..333: note = Synthetic: RNF43-38 (VL DNA)
  source 1..333: mol_type = other DNA
cagtttgtgcttactcagccaaactctgtgtctgcgaatctcggaagcacagtcaaactgtcttgcaatcgcagcactggtaacattggaagcaattatgtgagctggtaccagcagcatgagggaagatctcccaccactatgctttacagggatggtaagagaccagatggaattcctgacaggttctctggctccattgacggatcttccaactcagccctcctgacaatcaacaatgtgcagactgaagatgaagctgactacttctgtcagtcttacagtagtggtattaagtatattttcggcggtggaaccaagctcactgtccta

SEQ ID NO: 183 - protein, 123 aa, synthetic construct
  REGION 1..123: note = Synthetic: RNF43-38 (VH prot)
EVQLVESGGGLVQPGTSLKISCVASGFTFSDYWMSWVRQTPGKTMEWIGDIKSDGSYTNYAPSLKNRFTISRDNAKSTLYLQMNNVRSEDTATYYCTRDAYRYNYVHYGLDAWGQGASVTVSS

SEQ ID NO: 184 - protein, 111 aa, synthetic construct
  REGION 1..111: note = Synthetic: RNF43-38(VL prot)
QFVLTQPNSVSANLGSTVKLSCNRSTGNIGSNYVSWYQQHEGRSPTTMLYRDGKRPDGIPDRFSGSIDGSSNSALLTINNVQTEDEADYFCQSYSSGIKYIFGGGTKLTVL

SEQ ID NO: 185 - DNA, 363 bp, synthetic construct
  misc_feature 1..363: note = Synthetic: RNF43-41 (VH DNA)
  source 1..363: mol_type = other DNA
gaggtgcacctggtggagtctgggggaggcttagtgcagcctggaaggtccgtgaaactctcctgtacagcctcaggattcactttcagttactatgacatggcctgggtccgccaggctccaacgaagggtctggagtgggtcgcatcccttacttatgatggtagtagcacttactatcgagactccgtgaagggccgattcactatctccagagataatgcaaaaagcaccctatacctgcaaatggacagtctgaggtctgaggacacggccacttattactgtacaacagatcggggtagatacctaccctaccactttgattactggggccaaggagtcatggtcacagtctcctca

SEQ ID NO: 186 - DNA, 333 bp, synthetic construct
  misc_feature 1..333: note = Synthetic: RNF43-41 (VL DNA)
  source 1..333: mol_type = other DNA
gatgttgtgttgacacaaactccagtttccctgtctgtcacacttggagatcaagcttctatatcttgcaggtctagtcagagcctggaatatagtgatggatacacttatttggaatggtacctacagaagccaggccagtctccacagctcctcatctatgaagtttccacccgattttctggggtcccagacaggttcattggcagtgggtcagggacagatttcacccttaagatcagcagagtagagcctgaggacttgggagtttattactgcttccaagctacacatgatcccacgtttggagctgggaccaagctcgaactgaaa

SEQ ID NO: 187 - protein, 121 aa, synthetic construct
  REGION 1..121: note = Synthetic: RNF43-41 (VH prot)
EVHLVESGGGLVQPGRSVKLSCTASGFTFSYYDMAWVRQAPTKGLEWVASLTYDGSSTYYRDSVKGRFTISRDNAKSTLYLQMDSLRSEDTATYYCTTDRGRYLPYHFDYWGQGVMVTVSS

SEQ ID NO: 188 - protein, 111 aa, synthetic construct
  REGION 1..111: note = Synthetic: RNF43-41(VL prot)
DVVLTQTPVSLSVTLGDQASISCRSSQSLEYSDGYTYLEWYLQKPGQSPQLLIYEVSTRFSGVPDRFIGSGSGTDFTLKISRVEPEDLGVYYCFQATHDPTFGAGTKLELK

SEQ ID NO: 189 - DNA, 369 bp, synthetic construct
  misc_feature 1..369: note = Synthetic: RNF43-53 (VH DNA)
  source 1..369: mol_type = other DNA
gaggtgcagttggtggagtctgggggaggcttagtgcagcctggaaggtccctgaaactctcctgtggagcctcaggattcagtttcagtacctattacatggtctgggtccgccaggctccaacgaagggtctggagtgggtcgcatccattagtcctaatggtggtaacacttactatccagactccgtgaagggccgattcactatctccagagataatgcaaaaagcacccaatacctgcaaatggacagtctgaggtctgaggacacggccacttattactgtgcaacagatgggtcgtattactatgatggttatcggtggcttgcttactggggccaaggcactctggtcactgtctcttca

SEQ ID NO: 190 - DNA, 330 bp, synthetic construct
  misc_feature 1..330: note = Synthetic: RNF43-53 (VL DNA)
  source 1..330: mol_type = other DNA
cagtttgtgcttactcagccaaactctgtgtctacgaatctcggaagcacagtcaaactgtcttgcaagcgcagcactggtaacattggaagcaattatgtgagctggtaccagcagcatgagggaagatctcccaccactctgatttatagggatgataagagaccagatggagttcctgacaggttctctggctccattgacagatcttccaactcagccctcctgacaatcaataatgtgcagactgaagatgaagctggctacttctgtcagtctttcagtagtggtctgtttattttcggcggtggaaccaagctcactgtccta

SEQ ID NO: 191 - protein, 123 aa, synthetic construct
  REGION 1..123: note = Synthetic: RNF43-53 (VH prot)
EVQLVESGGGLVQPGRSLKLSCGASGFSFSTYYMVWVRQAPTKGLEWVASISPNGGNTYYPDSVKGRFTISRDNAKSTQYLQMDSLRSEDTATYYCATDGSYYYDGYRWLAYWGQGTLVTVSS

SEQ ID NO: 192 - protein, 110 aa, synthetic construct
  REGION 1..110: note = Synthetic: RNF43-53(VL prot)
QFVLTQPNSVSTNLGSTVKLSCKRSTGNIGSNYVSWYQQHEGRSPTTLIYRDDKRPDGVPDRFSGSIDRSSNSALLTINNVQTEDEAGYFCQSFSSGLFIFGGGTKLTVL

SEQ ID NO: 193 - DNA, 369 bp, synthetic construct
  misc_feature 1..369: note = Synthetic: RNF43-56 (VH DNA)
  source 1..369: mol_type = other DNA
gaagtacagcttgtggagtctggaggaggcttggtgcaacctgggacttctctgaaaatctcttgtgtagcctcgggattcactttcagtgactactggatgagctgggttcgccagactcctggaaggaccatggagtggattggagatattaaatatgatggcagttacacaaactatgcaccatccctaaagaatcgattcacaatctccagagacaatgccaagagtatcctgtacctgcagatgaacaatgtgagatctgaggacacagccacttattactgtactagagatgcatatgggtacaactacgtccactatggtttggatgcctggggccaaggagcttcagtcactgtctcctca

SEQ ID NO: 194 - DNA, 333 bp, synthetic construct
  misc_feature 1..333: note = Synthetic: RNF43-56 (VL DNA)
  source 1..333: mol_type = other DNA
cagtttgtgcttactcagccaaactctgtgtcttcgaatctcggaagcacagtcaaactgtcttgcaatcgcagcactggtaacattggaagcaattatgtgagttggtaccagcaacatgagggaagatctcccaccactatgatttatagggatgataagagaccagatggaattcctgacaggttctctggctccattgacggatcttccaactcggccctcctgacaatcaacaatgtgcagactgaagatgaagctgactacttctgtcagtcttacagtagtgatattaagtatattttcggcggtggaaccaagctcactgtccta

SEQ ID NO: 195 - protein, 123 aa, synthetic construct
  REGION 1..123: note = Synthetic: RNF43-56 (VH prot)
EVQLVESGGGLVQPGTSLKISCVASGFTFSDYWMSWVRQTPGRTMEWIGDIKYDGSYTNYAPSLKNRFTISRDNAKSILYLQMNNVRSEDTATYYCTRDAYGYNYVHYGLDAWGQGASVTVSS

SEQ ID NO: 196 - protein, 111 aa, synthetic construct
  REGION 1..111: note = Synthetic: RNF43-56(VL prot)
QFVLTQPNSVSSNLGSTVKLSCNRSTGNIGSNYVSWYQQHEGRSPTTMIYRDDKRPDGIPDRFSGSIDGSSNSALLTINNVQTEDEADYFCQSYSSDIKYIFGGGTKLTVL

SEQ ID NO: 197 - DNA, 372 bp, synthetic construct
  misc_feature 1..372: note = Synthetic: RNF43-61 (VH DNA)
  source 1..372: mol_type = other DNA
gaggtgcagctggtggagtctgggggagacttagtgcagcctggaaggtccctgacactctcctgtgcagcctcaggattcattttcagtgactattacatggcctgggtccgccaggctccaaagaagggtctggagtgggtcgcatccattacttatgagggcagtagtacctactacggagactccgtgaagggccgattcactatctccagagataatacaaaaagcaccctatatctgctaatgaacagtctgaggtctgacgactcggccacttactattgcgcaagacatggggcacactatacatataattacgcctactactttgattactggggccagggagtcatggtcacagtctcctca

SEQ ID NO: 198 - DNA, 321 bp, synthetic construct
  misc_feature 1..321: note = Synthetic: RNF43-61 (VL DNA)
  source 1..321: mol_type = other DNA
gacatcatgctgactcagtctccaactaccctgtctgtgattccaggagagaacatcagtctctcctgcagggccagtcagagtgttggtactaacttacattggtatcagcaaaaaccaagtgagtctccaagggttctcatcaactatgcttcccagtccatctctggaatcccctccaggttcagtggcagtggatcagggacagatttcactctcaatattaacagagtagagcctgaagattcttcagtttattactgtcaacagagtaagagctggccattcacgttcggctcaggaacgaagttggaaataaaa

SEQ ID NO: 199 - protein, 124 aa, synthetic construct
  REGION 1..124: note = Synthetic: RNF43-61 (VH prot)
EVQLVESGGDLVQPGRSLTLSCAASGFIFSDYYMAWVRQAPKKGLEWVASITYEGSSTYYGDSVKGRFTISRDNTKSTLYLLMNSLRSDDSATYYCARHGAHYTYNYAYYFDYWGQGVMVTVSS

SEQ ID NO: 200 - protein, 107 aa, synthetic construct
  REGION 1..107: note = Synthetic: RNF43-61(VL prot)
DIMLTQSPTTLSVIPGENISLSCRASQSVGTNLHWYQQKPSESPRVLINYASQSISGIPSRFSGSGSGTDFTLNINRVEPEDSSVYYCQQSKSWPFTFGSGTKLEIK

SEQ ID NO: 201 - DNA, 363 bp, synthetic construct
  misc_feature 1..363: note = Synthetic: RNF43-67 (VH DNA)
  source 1..363: mol_type = other DNA
gcggtgcagctggtggagtctgggggaggctcagtgcagcctggaaggtccatgaggctctcctgcgcagcctcaggattcactttcagtaactatgacatgacctgggtccgccaggctccaacgaagggtctggagtgggtcgcatccattacttctgatggtggtagtacttactctcgagactccgtgaagggccgattcactatctccagagataatgcaaaaagcaccctatacctgcaaatggacagtctgaggtctgaggacacggccacttattactgtacaacagatcggggtagatacctaccctactactttgattactggggccaaggagtcatggtcacagtctccgca

SEQ ID NO: 202 - DNA, 333 bp, synthetic construct
  misc_feature 1..333: note = Synthetic: RNF43-67 (VL DNA)
  source 1..333: mol_type = other DNA
gatgttgtgctgacacaaactccagtttccctgtctgtcacagttggagatcaagcttctatatcttgcaggtctagtcagagcctggaatatagtgatggatatagttatttggaatggtacctacagaagccaggccagtctccacagctcctcatctatgaagtttccagccgattttctggggtcccagacaggttcattggcagtgggtcagggacagatttcaccctcaagatcagcagagtagagcctgaggacttgggagtttattactgcttccaagctatacatgatcccacgtttggagctgggaccaagctggaactgaaa

SEQ ID NO: 203 - protein, 121 aa, synthetic construct
  REGION 1..121: note = Synthetic: RNF43-67 (VH prot)
AVQLVESGGGSVQPGRSMRLSCAASGFTFSNYDMTWVRQAPTKGLEWVASITSDGGSTYSRDSVKGRFTISRDNAKSTLYLQMDSLRSEDTATYYCTTDRGRYLPYYFDYWGQGVMVTVSA

SEQ ID NO: 204 - protein, 111 aa, synthetic construct
  REGION 1..111: note = Synthetic: RNF43-67(VL prot)
DVVLTQTPVSLSVTVGDQASISCRSSQSLEYSDGYSYLEWYLQKPGQSPQLLIYEVSSRFSGVPDRFIGSGSGTDFTLKISRVEPEDLGVYYCFQAIHDPTFGAGTKLELK

SEQ ID NO: 205 - DNA, 369 bp, synthetic construct
  misc_feature 1..369: note = Synthetic: RNF43-69 (VH DNA)
  source 1..369: mol_type = other DNA
gaggtgcagctggtggagtctgggggaggcttagtgcagcctggaaggtccctgaaactctcctgtgcagcctcaggattcactttcagtgactattacatggcctgggtccgccaggctccaaagaagggtctggagtgggtcgcatccattacttatgagggtagtagcacttactatggagactccgtgaagggccgattcactatctccagagataatgcaaaaagcaccctatacctgcaaatgaacagtctgaggtctgaggacacggccactttttattgtgcaagacatctggactatgggtatgactacgcctggtactttgacttctggggcccaggaaccatggtcaccgtgtcctca

SEQ ID NO: 206 - DNA, 321 bp, synthetic construct
  misc_feature 1..321: note = Synthetic: RNF43-69 (VL DNA)
  source 1..321: mol_type = other DNA
gacatcgtgctgactcagtctccaaccaccctgtctgtgactccaggagagacagtcagtctctcctgcaggactagccatagtattggcacaaatctacactggtatcagcaaaaaacaaatgagtctccaaggcttctcatcaagtatgcttcccagtccatctctgggatcccctccaggttcagtgccagtggatcagggacagattttactctcaacatcaacaatgtggagtttgatgatgtctcaagttatttttgtcaacagactcaaagctggcccctcacgttcggttctgggaccaagctggagatcaaa

SEQ ID NO: 207 - protein, 123 aa, synthetic construct
  REGION 1..123: note = Synthetic: RNF43-69 (VH prot)
EVQLVESGGGLVQPGRSLKLSCAASGFTFSDYYMAWVRQAPKKGLEWVASITYEGSSTYYGDSVKGRFTISRDNAKSTLYLQMNSLRSEDTATFYCARHLDYGYDYAWYFDFWGPGTMVTVSS

SEQ ID NO: 208 - protein, 107 aa, synthetic construct
  REGION 1..107: note = Synthetic: RNF43-69(VL prot)
DIVLTQSPTTLSVTPGETVSLSCRTSHSIGTNLHWYQQKTNESPRLLIKYASQSISGIPSRFSASGSGTDFTLNINNVEFDDVSSYFCQQTQSWPLTFGSGTKLEIK

SEQ ID NO: 209 - DNA, 360 bp, synthetic construct
  misc_feature 1..360: note = Synthetic: RNF43-71 (VH DNA)
  source 1..360: mol_type = other DNA
caggtcaagctgctgcagtctggggctgcactggtgaagcctggggactctgtgaagatgtcttgcaaagcttctggttatacattcactgaccacattatacactgggtgaagcagagtcatggaaagagccttgagtggattggttatattaatccttacagtggtagtactaactacaatgaaaagttcaagagcaaggccacattgactgtagacagatccaacaacacagcctatatggagtttagcagattgacatctgaggattctgcaatctattactgtgcaagaaagaactacggaggttatggagttatggatgcctggggtcaaggagcttcagtcactgtctcctca

SEQ ID NO: 210 - DNA, 318 bp, synthetic construct
  misc_feature 1..318: note = Synthetic: RNF43-71 (VL DNA)
  source 1..318: mol_type = other DNA
gacatccagatgacccagtctccttcactcctgtctgcatctgtgggagacagagtcactctcagctgcaaagcaagtcagaatatttataagaacttagcctggcatcagcaaaagcttggagaagctcccaaactcctgatttattatgcaaacagcttgcaaacgggcatcccatcaaggttcagtggcagtggatttggtacagattacacactcaccatcagcagcctgcagcctgaagatgttgccacatatttctgccagcagtcttctagcgggtggacgttcggtggaggcaccaagctggaattgaaa

SEQ ID NO: 211 - protein, 120 aa, synthetic construct
  REGION 1..120: note = Synthetic: RNF43-71 (VH prot)
QVKLLQSGAALVKPGDSVKMSCKASGYTFTDHIIHWVKQSHGKSLEWIGYINPYSGSTNYNEKFKSKATLTVDRSNNTAYMEFSRLTSEDSAIYYCARKNYGGYGVMDAWGQGASVTVSS

SEQ ID NO: 212 - protein, 106 aa, synthetic construct
  REGION 1..106: note = Synthetic: RNF43-71(VL prot)
DIQMTQSPSLLSASVGDRVTLSCKASQNIYKNLAWHQQKLGEAPKLLIYYANSLQTGIPSRFSGSGFGTDYTLTISSLQPEDVATYFCQQSSSGWTFGGGTKLELK

SEQ ID NO: 213 - DNA, 372 bp, synthetic construct
  misc_feature 1..372: note = Synthetic: RNF43-74 (VH DNA)
  source 1..372: mol_type = other DNA
gaggtgcagctggtggagtctgggggagacttagtgcagcctggaaggtccctgacactctcctgtgcagcctcaggattcactttcagtgactattacatggcctgggtccgccaggctccaaagaagggtctggagtgggtcgcatccattacttatgagggcagtagtacctactacggagactccgtgaagggccgattcactatctccagagataatacaaaaagcaccctatatctgctaatgaacagtctgaggtctgacgacacggccacttactattgcgcaagacatggggcacactatacatataattacgcctactactttgattactggggccagggagtcatggtcacagtctcctca

SEQ ID NO: 214 - DNA, 321 bp, synthetic construct
  misc_feature 1..321: note = Synthetic: RNF43-74 (VL DNA)
  source 1..321: mol_type = other DNA
gacatcatgctgactcagtctccaactaccctgtctgtgattccaggagagaacatcagtctctcctgcagggccagtcagagtgttggtactaacttacattggtatcagcaaaaaccaagtgagtctccaagggttctcatcaactatgcttcccagtccatctctggaatcccctccaggttcagtggcagtggatcagggacagatttcactctcaatattaacagagtagagcctgaagattcttcagtttattactgtcaacagagtaagagctggccattcacgttcggctcaggcacgaagttggaaataaaa

SEQ ID NO: 215 - protein, 124 aa, synthetic construct
  REGION 1..124: note = Synthetic: RNF43-74 (VH prot)
EVQLVESGGDLVQPGRSLTLSCAASGFTFSDYYMAWVRQAPKKGLEWVASITYEGSSTYYGDSVKGRFTISRDNTKSTLYLLMNSLRSDDTATYYCARHGAHYTYNYAYYFDYWGQGVMVTVSS

SEQ ID NO: 216 - protein, 107 aa, synthetic construct; identical to SEQ ID NO: 200
  REGION 1..107: note = Synthetic: RNF43-74(VL prot)

SEQ ID NO: 217 - DNA, 369 bp, synthetic construct
  misc_feature 1..369: note = Synthetic: RNF43-75 (VH DNA)
  source 1..369: mol_type = other DNA
gaggtgcagttggtggagtctgggggaggcttagtgcagcctggaaggtccctgaaactctcctgtgcagcctcaggattcactttcagtgaccattacatggcctgggtccgccaggctccaaagaagggtctggagtgggtcgcatccattacttatgagggtagtagcacttactatggagactccgtgaagggccgattcactatctccagagataatgcaaaaagcaccctatacctgcaaatgaacagtctgaggtctgaggacacggccactttttattgtgcaagacatctggactatgggtatgactacgcctggtcctttgacttctggggcccaggaaccatggtcaccgtgtcctca

SEQ ID NO: 218 - DNA, 321 bp, synthetic construct
  misc_feature 1..321: note = Synthetic: RNF43-75 (VL DNA)
  source 1..321: mol_type = other DNA
gacatcgtgttgactcagtctccaatcatcctgtctgtgactccaggagagacagtcagtctctcctgcaggactagccatagtattggcacaaatctacactggtatcagcaaaaaacaaatgagtctccaaggcttctcatcaactatgcttcccagtccatctctgggatcccctccaggttcagtgccagtggatcagggacagattttactctcaacatcaacaatgtggagtttgaagatgtcgcaagttattattgtcaacagactcaaaggtggcccctcacgttcggttctgggaccaagctggagatcaaa

SEQ ID NO: 219 - protein, 123 aa, synthetic construct
  REGION 1..123: note = Synthetic: RNF43-75 (VH prot)
EVQLVESGGGLVQPGRSLKLSCAASGFTFSDHYMAWVRQAPKKGLEWVASITYEGSSTYYGDSVKGRFTISRDNAKSTLYLQMNSLRSEDTATFYCARHLDYGYDYAWSFDFWGPGTMVTVSS

SEQ ID NO: 220 - protein, 107 aa, synthetic construct
  REGION 1..107: note = Synthetic: RNF43-75(VL prot)
DIVLTQSPIILSVTPGETVSLSCRTSHSIGTNLHWYQQKTNESPRLLINYASQSISGIPSRFSASGSGTDFTLNINNVEFEDVASYYCQQTQRWPLTFGSGTKLEIK

SEQ ID NO: 221 - DNA, 369 bp, synthetic construct; identical to SEQ ID NO: 181
  misc_feature 1..369: note = Synthetic: RNF43-76 (VH DNA)
  source 1..369: mol_type = other DNA

SEQ ID NO: 222 - DNA, 333 bp, synthetic construct
  misc_feature 1..333: note = Synthetic: RNF43-76 (VL DNA)
  source 1..333: mol_type = other DNA
cagtttgtgcttactcagccaaactctgtgtcttcgaatctcggaagcacagtcaaactgtcttgcaatcgcagcactggtaacattggaagcaattatgtgagctggtaccagcagcatgagggaagatctcccaccactatgatttacagggatggtaagagaccagatggaattcctgacaggttctctggctccattgacggatcttccaactcagccctcctgacaatcaacaatgtgcagactgaagatgaagctgactacttctgtcagtcttacagtagtggtattaagtatattttcggcggtggaaccaagctcactgtccta

SEQ ID NO: 223 - protein, 123 aa, synthetic construct; identical to SEQ ID NO: 183
  REGION 1..123: note = Synthetic: RNF43-76 (VH prot)

SEQ ID NO: 224 - protein, 111 aa, synthetic construct
  REGION 1..111: note = Synthetic: RNF43-76(VL prot)
QFVLTQPNSVSSNLGSTVKLSCNRSTGNIGSNYVSWYQQHEGRSPTTMIYRDGKRPDGIPDRFSGSIDGSSNSALLTINNVQTEDEADYFCQSYSSGIKYIFGGGTKLTVL

SEQ ID NO: 225 - DNA, 351 bp, synthetic construct
  misc_feature 1..351: note = Synthetic: RNF43-80 (VH DNA)
  source 1..351: mol_type = other DNA
gaggtgcagctggtggagtctggaggaggcttagtccagcctggaaggtccctgaaagtctcctgtttagcctctggattcaacttcggtgactttggaatgaattggattcgccaggctccagggaaggggctggagtgggtttcatatattagtagtaccagtggtacaatctactatgcggacactgtgaagggtcgcttcaccatttccagagataatgccaagaacaccctttatctccaactgagcagtctgaggtctgaggacaccgccttgtattactgtgcaagaccggcgggaaggtactttgattactggggccaaggagtcttggtcacagtctcctca

SEQ ID NO: 226 - DNA, 339 bp, synthetic construct
  misc_feature 1..339: note = Synthetic: RNF43-80 (VL DNA)
  source 1..339: mol_type = other DNA
gacattgtgatgacccaatctccatcctctctggctgtgtcagcaggagagacggtcactatagactgcaagtccaatcagagtcttttatacagtggaaaccaaaagaactacttggcctggtaccagcagaaaccagggcagtctcctaaactgctgatctactggacttctactaggcaatctggtgtccctgatcgcttcataggcagtggatctgggacagagttcactctgaccatcagcagtgtgcaggcagaagatctggcaatatattactgtcagcagtattatcacactccattcacgttcggctcagggacgaaattggaaataaaa

SEQ ID NO: 227 - protein, 117 aa, synthetic construct
  REGION 1..117: note = Synthetic: RNF43-80 (VH prot)
EVQLVESGGGLVQPGRSLKVSCLASGFNFGDFGMNWIRQAPGKGLEWVSYISSTSGTIYYADTVKGRFTISRDNAKNTLYLQLSSLRSEDTALYYCARPAGRYFDYWGQGVLVTVSS

SEQ ID NO: 228 - protein, 113 aa, synthetic construct
  REGION 1..113: note = Synthetic: RNF43-80(VL prot)
DIVMTQSPSSLAVSAGETVTIDCKSNQSLLYSGNQKNYLAWYQQKPGQSPKLLIYWTSTRQSGVPDRFIGSGSGTEFTLTISSVQAEDLAIYYCQQYYHTPFTFGSGTKLEIK

SEQ ID NO: 229 - DNA, 372 bp, synthetic construct
  misc_feature 1..372: note = Synthetic: RNF43-86 (VH DNA)
  source 1..372: mol_type = other DNA
gaggtgcagctggtggagtctgggggaggcttagtgcagcctggaaggtccctgaaactctcctgtgcagcctcaggattcactttcactaactattacatggcctgggtccgccaggctccaacgaagggtctggagtgggtcgcgtccatcagtcctagtggtgataccacttactatccagactccgtgaagggccgattcactgtctccagagataatgcaaaaagtacccaatacctgcaaatggacaatctgaggtctgaggacacggccacttattactgtgcaagagacgcatatacatactatggatataagggctggtactttgacttctggggcccaggaaccatggtcaccgtgtcctca

SEQ ID NO: 230 - DNA, 330 bp, synthetic construct
  misc_feature 1..330: note = Synthetic: RNF43-86 (VL DNA)
  source 1..330: mol_type = other DNA
cagtttgtgcttactcagccaaactctgtgtctacgaatctcggaagtgcagtcaaactgtcttgcaagcgcagcactgatgacattggaagcaattatgtgaactggtaccagcagcatgagggaagatctcccaccactatgatttatagggatgataagagaccagatggagttcctgacaggttctctggctccattgacagatcttccaactcagccctcctgacaatcaataatgtgcagactgaagatgaggctgactacttctgtcagtcttacagtagtggtatttatattttcggcggtggaaccaagctcactgtcctg

SEQ ID NO: 231 - protein, 124 aa, synthetic construct
  REGION 1..124: note = Synthetic: RNF43-86 (VH prot)
EVQLVESGGGLVQPGRSLKLSCAASGFTFTNYYMAWVRQAPTKGLEWVASISPSGDTTYYPDSVKGRFTVSRDNAKSTQYLQMDNLRSEDTATYYCARDAYTYYGYKGWYFDFWGPGTMVTVSS

SEQ ID NO: 232 - protein, 110 aa, synthetic construct
  REGION 1..110: note = Synthetic: RNF43-86(VL prot)
QFVLTQPNSVSTNLGSAVKLSCKRSTDDIGSNYVNWYQQHEGRSPTTMIYRDDKRPDGVPDRFSGSIDRSSNSALLTINNVQTEDEADYFCQSYSSGIYIFGGGTKLTVL

SEQ ID NO: 233 - DNA, 357 bp, synthetic construct
  misc_feature 1..357: note = Synthetic: RNF43-90 (VH DNA)
  source 1..357: mol_type = other DNA
caggtacagttgaaggagtcaggacctggtctggtgcagccctcacagaccctgtccctcacctgcactgtctctggattctcacttaccagctacagtgtacactgggttcgccaacattcaggaaaccgtctggaatggatgggaagaatgtggagtgctggagacacatcatttaattcagcgttcacatcccgactgaacatcaccagggacacctccaagagccaagttttcttaaaaatgaacagtctgcaaactgaagacacaggcacttactactgtgtcagatccttctataatgcggggtatccttttgattactggggccagggagtcatggtcacagtctcctca

SEQ ID NO: 234 - DNA, 315 bp, synthetic construct
  misc_feature 1..315: note = Synthetic: RNF43-90 (VL DNA)
  source 1..315: mol_type = other DNA
gacactgtactgacccagtctcctgctttggctgtgtctccaggagagagggttactatctcctgtagggccagtgatagtgtcattaaacttatgaactggtatcaacagaaaccaggacagcgacccaaactcctcatctatctggcatcacacctagcatctggggtccctgccaggttcagtggcagtgggtctgggacagacttcaccctcaccattgatcctgtggagactgatgacactgcaacctattactgtcagcagagtaggaatgatcccacgtttggacctgggaccaagctggaactgaaa

SEQ ID NO: 235 - protein, 119 aa, synthetic construct
  REGION 1..119: note = Synthetic: RNF43-90 (VH prot)
QVQLKESGPGLVQPSQTLSLTCTVSGFSLTSYSVHWVRQHSGNRLEWMGRMWSAGDTSFNSAFTSRLNITRDTSKSQVFLKMNSLQTEDTGTYYCVRSFYNAGYPFDYWGQGVMVTVSS

SEQ ID NO: 236 - protein, 105 aa, synthetic construct
  REGION 1..105: note = Synthetic: RNF43-90(VL prot)
DTVLTQSPALAVSPGERVTISCRASDSVIKLMNWYQQKPGQRPKLLIYLASHLASGVPARFSGSGSGTDFTLTIDPVETDDTATYYCQQSRNDPTFGPGTKLELK

SEQ ID NO: 237 - DNA, 354 bp, synthetic construct
  misc_feature 1..354: note = Synthetic: ZNRF3-101 (VH DNA)
  source 1..354: mol_type = other DNA
caggtgcaactgaaggagtcaggacctggtctggtgcagcccacacagaccctgtccctcacctgcactgtctctgggttttcactgaccagctctactgtaagctgggttcgccaacctccaggaaagggtctggagtggattgcagcaatatcaagtgctgaaaacacatattataattcaggtctcaaatcccgactgagcatcagcagggacacctccaaaagccaagttttcttaaaaatgaacagtctgcaaactgaagacacagccatgtacttctgtgccggcggtagaatactggagaccctctttgattcctggggccaaggagtcatggtcacagtctcctca

SEQ ID NO: 238 - DNA, 330 bp, synthetic construct
  misc_feature 1..330: note = Synthetic: ZNRF3-101 (VL DNA)
  source 1..330: mol_type = other DNA
gacattgtgctgacccagtctcctgctttggctgtgtctctagggcagagggccaccatctcttgcaagaccaaccagaatgtcgatttttatggcaaaacttatatgcactggtaccaacagaaacggggacaacaacccaaactcctcatctatttagcatccaacttagcatctgggatccctgccaggttcagtggtagagggtctgggacagacttcaccctcaccattgatcctgtggcgactgatgatactgcaacctattactgtcagcagactaggagtcttcctcccacgtttggagctgggaccaagctggaactgaaa

SEQ ID NO: 239 - protein, 118 aa, synthetic construct
  REGION 1..118: note = Synthetic: ZNRF3-101 (VH prot)
QVQLKESGPGLVQPTQTLSLTCTVSGFSLTSSTVSWVRQPPGKGLEWIAAISSAENTYYNSGLKSRLSISRDTSKSQVFLKMNSLQTEDTAMYFCAGGRILETLFDSWGQGVMVTVSS

SEQ ID NO: 240 - protein, 110 aa, synthetic construct
  REGION 1..110: note = Synthetic: ZNRF3-101(VL prot)
DIVLTQSPALAVSLGQRATISCKTNQNVDFYGKTYMHWYQQKRGQQPKLLIYLASNLASGIPARFSGRGSGTDFTLTIDPVATDDTATYYCQQTRSLPPTFGAGTKLELK

SEQ ID NO: 241 - DNA, 369 bp, synthetic construct
  misc_feature 1..369: note = Synthetic: ZNRF3-117 (VH DNA)
  source 1..369: mol_type = other DNA
gagatacagctgcaggagtcaggacctggccttgtgaaaccttcacagtcactctccctcacctgttctgtcactggttacaccattaccagtgattatgattggagctggatccggaagttcccaggaaataaaatggagtggatgggatacataaactacagtggtagcactaactacaacccatcgctcaaaagtcgaatctcctttaccagagacacatccaagaatcagttcttcctgcagttgaactctataactactgacgatacagccacatattactgtgcaagatatggtccatactacgggttttccgactggtactttgacttctggggcccgggaaccatggtcaccgtgtcctca

SEQ ID NO: 242 - DNA, 321 bp, synthetic construct
  misc_feature 1..321: note = Synthetic: ZNRF3-117 (VL DNA)
  source 1..321: mol_type = other DNA
gatgtccagatgacccagtctccgtcttatcttgctgcgtctcctggagaaagtgtttccatcagttgcaaggcaagtcagagcattagtaattatttagcctggtatcaacagaaacctggaaaaacatataggcttcttatctactctgggtcaactttgcaatctggaactccatcaaggttcagtggcagtggatctggtacagatttcactctcaccatcagaagcctggagcctgaagattttggactctattactgtcaacagtattatgaaaaaccgctcacgttcggttctgggaccaagctggagatcaaa

SEQ ID NO: 243 - protein, 123 aa, synthetic construct
  REGION 1..123: note = Synthetic: ZNRF3-117 (VH prot)
EIQLQESGPGLVKPSQSLSLTCSVTGYTITSDYDWSWIRKFPGNKMEWMGYINYSGSTNYNPSLKSRISFTRDTSKNQFFLQLNSITTDDTATYYCARYGPYYGFSDWYFDFWGPGTMVTVSS

SEQ ID NO: 244 - protein, 107 aa, synthetic construct
  REGION 1..107: note = Synthetic: ZNRF3-117(VL prot)
DVQMTQSPSYLAASPGESVSISCKASQSISNYLAWYQQKPGKTYRLLIYSGSTLQSGTPSRFSGSGSGTDFTLTIRSLEPEDFGLYYCQQYYEKPLTFGSGTKLEIK

SEQ ID NO: 245 - DNA, 357 bp, synthetic construct
  misc_feature 1..357: note = Synthetic: ZNRF3-128 (VH DNA)
  source 1..357: mol_type = other DNA
gaggtacagctggtggagtctggaggaggcttagtgcagcctggaaagtccctgaaactctcctgttcagcctctggattcacattcagtagctatggcatgcactggattcgccaagctccaggaaaggggctagattgggttgcatacattagtactaacagcgctacagtctatgcagacgctgtgaagggccggttcaccatctccagagacaatgcaaagaacaccctgtacctacaactcaacagtctgaagtctgaggacactgccatctattactgtgcaagaagcggctataccacctatatgccctttacttactggggccaaggcactctggtcactgtctcttca

SEQ ID NO: 246 - DNA, 339 bp, synthetic construct
  misc_feature 1..339: note = Synthetic: ZNRF3-128 (VL DNA)
  source 1..339: mol_type = other DNA
gatgttttgatgacacaaactccagtctccctgcctgtcagccttggaggtcaagtctctatctcttgccggtcaagtcagagccttgtacacagtgatggaaacacctacttgcattggtatctgcagaagccaggccagtctccacagctcctcatctatcgggtttccaacagattttctggggtgtcagacaggttcagtggcagtgggtcagggacagatttcaccctcaagatcagcagagtagagcctgacgacttgggaatttattactgcttacaaactacacattttctcacgtggacgttcggtggaggcaccaagctggaattgaaa

SEQ ID NO: 247 - protein, 119 aa, synthetic construct
  REGION 1..119: note = Synthetic: ZNRF3-128 (VH prot)
EVQLVESGGGLVQPGKSLKLSCSASGFTFSSYGMHWIRQAPGKGLDWVAYISTNSATVYADAVKGRFTISRDNAKNTLYLQLNSLKSEDTAIYYCARSGYTTYMPFTYWGQGTLVTVSS

SEQ ID NO: 248 - protein, 113 aa, synthetic construct
  REGION 1..113: note = Synthetic: ZNRF3-128(VL prot)
DVLMTQTPVSLPVSLGGQVSISCRSSQSLVHSDGNTYLHWYLQKPGQSPQLLIYRVSNRFSGVSDRFSGSGSGTDFTLKISRVEPDDLGIYYCLQTTHFLTWTFGGGTKLELK

SEQ ID NO: 249 - DNA, 357 bp, synthetic construct
  misc_feature 1..357: note = Synthetic: ZNRF3-131 (VH DNA)
  source 1..357: mol_type = other DNA
gaggtgcaggtggtggagtctgggggaggcctagtgcagcctggaagatccctgaaagtctcctgtgcagcctcaggattcactttcagtagatatggcatggcttgggtccgccaggctccgacgaagggtctggagtgggtcgcatccattagtactggtggtgaaaacacttactatcgagactccgtgaagggccgattcactatctccagagataatgtaaaaaacaccctatacctgcaaatggacagtctgaggtctgaggacacggccacttattattgtgcaagacatgggcgggtactaaattactttgattactggggccaaggagtcatggtcacagtctcttta

SEQ ID NO: 250 - DNA, 321 bp, synthetic construct
  misc_feature 1..321: note = Synthetic: ZNRF3-131 (VL DNA)
  source 1..321: mol_type = other DNA
gacatccagatgacacagtctccagcttccctgtctgcatctctgggagaaactgtctccatcgaatgtctaacaagtgaggacatatacacttatttagcatggtatcagcagaagccagggaaatctcctcacctcctgatctttgctgcaagtaggttgcaagatggggtcccatcacggttcagtggcagtggatctggcacacagtattctctcaagatcagcggcatgcaacctgaagatgaaggggattatttctgtctacagggttccaagtttccgtacacgtttggagctgggaccaagctggacctgaaa

SEQ ID NO: 251 - protein, 119 aa, synthetic construct
  REGION 1..119: note = Synthetic: ZNRF3-131 (VH prot)
EVQVVESGGGLVQPGRSLKVSCAASGFTFSRYGMAWVRQAPTKGLEWVASISTGGENTYYRDSVKGRFTISRDNVKNTLYLQMDSLRSEDTATYYCARHGRVLNYFDYWGQGVMVTVSL

SEQ ID NO: 252 - protein, 107 aa, synthetic construct
  REGION 1..107: note = Synthetic: ZNRF3-131(VL prot)
DIQMTQSPASLSASLGETVSIECLTSEDIYTYLAWYQQKPGKSPHLLIFAASRLQDGVPSRFSGSGSGTQYSLKISGMQPEDEGDYFCLQGSKFPYTFGAGTKLDLK

SEQ ID NO: 253 - DNA, 372 bp, synthetic construct
  misc_feature 1..372: note = Synthetic: ZNRF3-163 (VH DNA)
  source 1..372: mol_type = other DNA
gaggtgcagttggaggagtctgggggaggcttagtgcagcctggaaggtccatgaaattctcctgtgcagcctcaggattcactttcagtaactattacatggcctgggtccgccaggctccatcgaagggtctggagtgggtcgcatccattagtactggtggaggtgatgtttactatcgagactccgtgaagggccgattcacgatctccagagataatgcaaaaagcaccctatacctgcaaatggacagtctgaggtctgaggacacggccacttattactgtgcaagacattcggggggtagaattcgggcccggggtcactactttgattactggggccaaggagtcatggtcacagtctcctca

SEQ ID NO: 254 - DNA, 321 bp, synthetic construct
  misc_feature 1..321: note = Synthetic: ZNRF3-163 (VL DNA)
  source 1..321: mol_type = other DNA
gacatccagatgacacagtctccagcttccctgtctgcatctcttggagaaactgtctccatcgaatgtctagcaagtgaggacatttccaatgatttagcgtggtatcagcagaagtcagggaaatctcctcaggtcctgatctatggtgtaagcaggttgcaagacggggtcccatcacggttcagtggcagtggatctggcacacggtattctctcaagatcagcggcatgcaacctgaagatgaagcagattatttctgtcaacagagttacaagtatccgtacacgtttggagctgggaccaagctggaactgaaa

SEQ ID NO: 255 - protein, 124 aa, synthetic construct
  REGION 1..124: note = Synthetic: ZNRF3-163 (VH prot)
EVQLEESGGGLVQPGRSMKFSCAASGFTFSNYYMAWVRQAPSKGLEWVASISTGGGDVYYRDSVKGRFTISRDNAKSTLYLQMDSLRSEDTATYYCARHSGGRIRARGHYFDYWGQGVMVTVSS

SEQ ID NO: 256 - protein, 107 aa, synthetic construct
  REGION 1..107: note = Synthetic: ZNRF3-163(VL prot)
DIQMTQSPASLSASLGETVSIECLASEDISNDLAWYQQKSGKSPQVLIYGVSRLQDGVPSRFSGSGSGTRYSLKISGMQPEDEADYFCQQSYKYPYTFGAGTKLELK

SEQ ID NO: 257 - DNA, 363 bp, synthetic construct
  misc_feature 1..363: note = Synthetic: ZNRF3-17 (VH DNA)
  source 1..363: mol_type = other DNA
gaggtgcagctggtggagtctgggggaggcttagtgcagcctggagggtccctgaaactctcctgtgcagcctcaggattcactttcagtaattatgacatggcctgggtccgccaggctccaacgaagggtctggagtgggtcgcatccattagtactggtggtggtaaaacttactatcgagactccgtgaagggccgactcactatctccagagataatgcaaaaaacacccaatacctgcaaatggacagtctgaggtctgaggacacggccacttattactgtgcaagactcggtccggcatactccggggagtggtttgcttactggggccaaggcactctggtcactgtctcttca

SEQ ID NO: 258 - DNA, 318 bp, synthetic construct
  misc_feature 1..318: note = Synthetic: ZNRF3-17 (VL DNA)
  source 1..318: mol_type = other DNA
gacactgtgctgacccagtctcctgctttggctgtgtctccaggagagagggttaccatctcctgtggggccagtgaaggtgtcaatacacgtatgcactggtaccaacagagatcaggacaacaacccaaactcctcatctacggttcctccaacctagattctggagtccctgacaggttcagtggcagtgggtctgggacagactttaccctcaccatcgatcctgtggaggctaaagatgctgcaacctatttctgtcagcagagttggaatgttccgaacacgtttggagctgggaccaaactggaactgaaa

SEQ ID NO: 259 - protein, 121 aa, synthetic construct
  REGION 1..121: note = Synthetic: ZNRF3-17 (VH prot)
EVQLVESGGGLVQPGGSLKLSCAASGFTFSNYDMAWVRQAPTKGLEWVASISTGGGKTYYRDSVKGRLTISRDNAKNTQYLQMDSLRSEDTATYYCARLGPAYSGEWFAYWGQGTLVTVSS

SEQ ID NO: 260 - protein, 106 aa, synthetic construct
  REGION 1..106: note = Synthetic: ZNRF3-17(VL prot)
DTVLTQSPALAVSPGERVTISCGASEGVNTRMHWYQQRSGQQPKLLIYGSSNLDSGVPDRFSGSGSGTDFTLTIDPVEAKDAATYFCQQSWNVPNTFGAGTKLELK

SEQ ID NO: 261 - DNA, 357 bp, synthetic construct
  misc_feature 1..357: note = Synthetic: ZNRF3-170 (VH DNA)
  source 1..357: mol_type = other DNA
gaggtgcagctggtggagtctgggggaggcttagtgcagcctggaaggaccctgaaactctcctgtgcagcctcaggattcactttcagtgactattacatggcctgggtccgccaggctccaaagaagggtctggagtgggtcgcatccattagttctgggggttttagtatttattatggagactccgtgaagggccgattcactatctccagagataatgcaaaaaccaccctatacctgcaaatgaacagtctgaggtctgaggacacggccacttattattgtgcaagacatacgattagtacggccttttttgattactggggccaaggagtcatggtcacagtctcctca

SEQ ID NO: 262 - DNA, 321 bp, synthetic construct
  misc_feature 1..321: note = Synthetic: ZNRF3-170 (VL DNA)
  source 1..321: mol_type = other DNA
gacactgtgctgacccagtctcctgctttggctgtgtctccaggagagagggttaccatctcctgtaaggccaatgaaaatgtcagtacacgtatgcactggtaccaacagagaccaggacagcgacccaaactcctcatctataaagcatcaaacctaggatctggggtccctgccaggttcagtggcagtgggtctgggacagacttcaccctcaccattgatcctgtggaggctgatgacactgcaacctatttctgtcagcagagttggaatggtcctccgtggacgttcggtggaggcaccaagctggagttgaaa

SEQ ID NO: 263 - protein, 119 aa, synthetic construct
  REGION 1..119: note = Synthetic: ZNRF3-170 (VH prot)
EVQLVESGGGLVQPGRTLKLSCAASGFTFSDYYMAWVRQAPKKGLEWVASISSGGFSIYYGDSVKGRFTISRDNAKTTLYLQMNSLRSEDTATYYCARHTISTAFFDYWGQGVMVTVSS

SEQ ID NO: 264 - protein, 107 aa, synthetic construct
  REGION 1..107: note = Synthetic: ZNRF3-170(VL prot)
DTVLTQSPALAVSPGERVTISCKANENVSTRMHWYQQRPGQRPKLLIYKASNLGSGVPARFSGSGSGTDFTLTIDPVEADDTATYFCQQSWNGPPWTFGGGTKLELK

SEQ ID NO: 265 - DNA, 360 bp, synthetic construct
  misc_feature 1..360: note = Synthetic: ZNRF3-171 (VH DNA)
  source 1..360: mol_type = other DNA
caggtccagttgcaccaatctggaactgaactggccagacctgggacttcagtgaagctgtcctgcaaggcttctggctataccttcagcaggttttatattaactgggtgaagaagaggcctggacagggccttgagtggatcggatatattgatcctggaaatggtggtactttccacagtgagaagttcaaggacaaagcctcacttactgcagacacatcttccagcacagcctacatggtactgggcagcctggcacctgaagactctgcattctatttctgtgtaaggggttactatagcagctatgattggtttgcttactggggccaaggcactctggtcactgtctcttca

SEQ ID NO: 266 - DNA, 321 bp, synthetic construct
  misc_feature 1..321: note = Synthetic: ZNRF3-171 (VL DNA)
  source 1..321: mol_type = other DNA
gacatccagttgacccagtctccatcctccatgtctgcatctctgggagacagagtcagtcttacttgccagtcaagccagggcattggaaattatttaagctggtaccagcataaaccaggggaacctcctaagcctatactttattatgcaaccaacttggcagatggggtcccatcaagattcagtggcagtaggtctgggtcagattattctctcaccatcagcagcctggagtctgaagatacagcaatctattactgtctacagtatgatgagtacccttacacgtttggacctgggaccaagctggaactgaga

SEQ ID NO: 267 - protein, 120 aa, synthetic construct
  REGION 1..120: note = Synthetic: ZNRF3-171 (VH prot)
QVQLHQSGTELARPGTSVKLSCKASGYTFSRFYINWVKKRPGQGLEWIGYIDPGNGGTFHSEKFKDKASLTADTSSSTAYMVLGSLAPEDSAFYFCVRGYYSSYDWFAYWGQGTLVTVSS

SEQ ID NO: 268 - protein, 107 aa, synthetic construct
  REGION 1..107: note = Synthetic: ZNRF3-171(VL prot)
DIQLTQSPSSMSASLGDRVSLTCQSSQGIGNYLSWYQHKPGEPPKPILYYATNLADGVPSRFSGSRSGSDYSLTISSLESEDTAIYYCLQYDEYPYTFGPGTKLELR

SEQ ID NO: 269 - DNA, 360 bp, synthetic construct
  misc_feature 1..360: note = Synthetic: ZNRF3-172 (VH DNA)
  source 1..360: mol_type = other DNA
gaggtgcagctggcggagtctgggggaggcttagagcagcctggaaggtccctgaaactctcctgtgcagcctcaggattcactttcagtaactatgacatggcctgggtccgccaggctccaacgaagggtctggagtgggtcgcatccattattgctagtggtggtactacttattatcgagactccgtgaagggccgattcactgtctccagagataatgcaaaaagcaccctatacctacaaatggacagtctgaggtctgaggacacggccacttattactgtgcaagacatggggtcggtagctacgattggtttgctgactggggccaaggcactctggtcactgtctcttca

SEQ ID NO: 270 - DNA, 327 bp, synthetic construct
  misc_feature 1..327: note = Synthetic: ZNRF3-172 (VL DNA)
  source 1..327: mol_type = other DNA
gacattgtcttgacccagtctcctgctttggctgtgtctctagggcagagggccacaatctcctgtagggccagccaaagtgtcactatatctggatttaatcttatgcactggtaccaacagaaaccaggacaacaacccaagctcctcatctatcgtgcatccaacctagcatttgggatccctgccaggttcagtggcagtgggtctgggacagacttcaccctcaccatcaatcctgtgcaggctgatgatattacaacctattactgtcagcaaagtaggaagtctcggacgttcggtggaggcaccaaactggaattgaaa

SEQ ID NO: 271 - protein, 120 aa, synthetic construct
  REGION 1..120: note = Synthetic: ZNRF3-172 (VH prot)
EVQLAESGGGLEQPGRSLKLSCAASGFTFSNYDMAWVRQAPTKGLEWVASIIASGGTTYYRDSVKGRFTVSRDNAKSTLYLQMDSLRSEDTATYYCARHGVGSYDWFADWGQGTLVTVSS

SEQ ID NO: 272 - protein, 109 aa, synthetic construct
  REGION 1..109: note = Synthetic: ZNRF3-172(VL prot)
DIVLTQSPALAVSLGQRATISCRASQSVTISGFNLMHWYQQKPGQQPKLLIYRASNLAFGIPARFSGSGSGTDFTLTINPVQADDITTYYCQQSRKSRTFGGGTKLELK

SEQ ID NO: 273 - DNA, 360 bp, synthetic construct
  misc_feature 1..360: note = Synthetic: ZNRF3-179 (VH DNA)
  source 1..360: mol_type = other DNA
gaggtgcagctggcggagtctgggggaggcttagagcagcctggaaggtccctgaaactctcctgtgcagcctcaggattcactttcagtaactatgacatggcctgggtccgccaggctccaacgaagggtctggagtgggtcgcatccattattactagtggtgatactacttattatcgagactccgtgaagggccgattcactgtctccagagataatgcaaaaagcaccctatacctgcaaatggacagtctgaggtctgaggacacggccacttatcactgtgcaagacatggggtcggtagctacgattggtttgctgactggggccaaggcactctggtcactgtctcttca

SEQ ID NO: 274 - DNA, 327 bp, synthetic construct
  misc_feature 1..327: note = Synthetic: ZNRF3-179 (VL DNA)
  source 1..327: mol_type = other DNA
gacattgtcttgacccagtctcctgctttggctgtgtctctagggcagagggccacaatctcctgtagggccagccaaagtgtcactatatctggatataatcttatgcactggtaccaacagaaaccaggacaacaacccaaactcctcatctatcgtgcatccaacctagcatttgggatccctgccaggttcagtggcagtgggtctgggacagacttcaccctcaccatcaatcctgtgcaggctgatgatattacaacctattactgtcagcagagtaggaagtctcggacgttcggtggaggcaccaaactggaattgaaa

SEQ ID NO: 275 - protein, 120 aa, synthetic construct
  REGION 1..120: note = Synthetic: ZNRF3-179 (VH prot)
EVQLAESGGGLEQPGRSLKLSCAASGFTFSNYDMAWVRQAPTKGLEWVASIITSGDTTYYRDSVKGRFTVSRDNAKSTLYLQMDSLRSEDTATYHCARHGVGSYDWFADWGQGTLVTVSS

SEQ ID NO: 276 - protein, 109 aa, synthetic construct
  REGION 1..109: note = Synthetic: ZNRF3-179(VL prot)
DIVLTQSPALAVSLGQRATISCRASQSVTISGYNLMHWYQQKPGQQPKLLIYRASNLAFGIPARFSGSGSGTDFTLTINPVQADDITTYYCQQSRKSRTFGGGTKLELK

SEQ ID NO: 277 - DNA, 360 bp, synthetic construct
  misc_feature 1..360: note = Synthetic: ZNRF3-182 (VH DNA)
  source 1..360: mol_type = other DNA
gaggtgcagctggcggagtctgggggaggcttagagcagcctggaaggtccctgaaactctcctgtgcagcctcaggattcactttcagtaactatgacatggcctgggtccgccaggctccaacgaagggtctggagtgggtcgcatccattattaaaagtggtgatacttcttattatcgagactccgtgaagggccgattcactgtctccagagataatgcaaaaagcaccctatacctgcaaatggacagtctgaggtctgaggacacggccacttattactgtgcaagacatggggtcggtagctacgattggtttgctgactggggccaaggcactctggtcactgtctcttca

SEQ ID NO: 278 - DNA, 327 bp, synthetic construct
  misc_feature 1..327: note = Synthetic: ZNRF3-182 (VL DNA)
  source 1..327: mol_type = other DNA
gacattgtcttgacccagtctcctgctttggctgtgtctctagggcagagggccacaatctcctgtagggccagccaaagtgtcactatatctggatttaatcttatgcactggtaccaacagaaaccaggacaacaacccaaactcctcatctatcgtgcatccaacctagcatttgggatccctgccaggttcagtggcagtgggtctgggacagacttcaccctcaccatcaatcctgtgcaggctgatgattttacaacctattactgtcagcagagtaggaagtctcggacgttcggtggaggcaccaaactggaattgaaa

SEQ ID NO: 279 - protein, 120 aa, synthetic construct
  REGION 1..120: note = Synthetic: ZNRF3-182 (VH prot)
EVQLAESGGGLEQPGRSLKLSCAASGFTFSNYDMAWVRQAPTKGLEWVASIIKSGDTSYYRDSVKGRFTVSRDNAKSTLYLQMDSLRSEDTATYYCARHGVGSYDWFADWGQGTLVTVSS

SEQ ID NO: 280 - protein, 109 aa, synthetic construct
  REGION 1..109: note = Synthetic: ZNRF3-182(VL prot)
DIVLTQSPALAVSLGQRATISCRASQSVTISGFNLMHWYQQKPGQQPKLLIYRASNLAFGIPARFSGSGSGTDFTLTINPVQADDFTTYYCQQSRKSRTFGGGTKLELK

SEQ ID NO: 281 - DNA, 351 bp, synthetic construct
  misc_feature 1..351: note = Synthetic: ZNRF3-195 (VH DNA)
  source 1..351: mol_type = other DNA
gaggtacagctggtggagtctggaggaggcttagtgcagcctggaaactccctgaacctctcctgttcagcctctggattcccattcagtacctatggcatgcactggattcgccaagctccaggaaaggggctagattgggttgcatacatttcttataacagcggtacagtctatgcagacgctgtgaagggccggttcaccatctccagagacaatgcaaagaacaccctgtacctgcaactcaacagtctgaagtctgaagacactgccatctattactgtgcaagaggttactatgatggttattatcgttactggggccaaggagtcatggtcacagtctcctcg

SEQ ID NO: 282 - DNA, 336 bp, synthetic construct
  misc_feature 1..336: note = Synthetic: ZNRF3-195 (VL DNA)
  source 1..336: mol_type = other DNA
gacattgtgatgacccagtctccattctccctggctgtgtcagaaggagagatggtcactataaactgcaagtccagtcagagtcttttatccagtggaaaccaaaagaactacttggcctggtaccagcagagaccagggcagtctcctaaactactgatctaccatgcatccactaggcaatcaggggtccctgatcgcttcattggcagtggatctgggacagacttcactctgaccatcagcgatgtgcaggctgaagacctggcagattattactgcctacaacattacagctctcccacgttcggctcagggacgaagttggagataaaa

SEQ ID NO: 283 - protein, 117 aa, synthetic construct
  REGION 1..117: note = Synthetic: ZNRF3-195 (VH prot)
EVQLVESGGGLVQPGNSLNLSCSASGFPFSTYGMHWIRQAPGKGLDWVAYISYNSGTVYADAVKGRFTISRDNAKNTLYLQLNSLKSEDTAIYYCARGYYDGYYRYWGQGVMVTVSS

SEQ ID NO: 284 - protein, 112 aa, synthetic construct
  REGION 1..112: note = Synthetic: ZNRF3-195(VL prot)
DIVMTQSPFSLAVSEGEMVTINCKSSQSLLSSGNQKNYLAWYQQRPGQSPKLLIYHASTRQSGVPDRFIGSGSGTDFTLTISDVQAEDLADYYCLQHYSSPTFGSGTKLEIK

SEQ ID NO: 285 - DNA, 360 bp, synthetic construct
  misc_feature 1..360: note = Synthetic: ZNRF3-219 (VH DNA)
  source 1..360: mol_type = other DNA
gaggtgcagctggtggagtctgggggaggcctagtgcagcctggaaggtccctgaaagtctcctgtgcagcctcaggattcactttcagtacctttgcaatggcctgggtgcgccaggctccaaagaagggtctggagtgggttgcaaccattactcctggtggtagtaacacttactatccagactccgtgaaaggccgattcactatctccagagataatgcaaaaagcaccctatacctgcaaatggacagtctgaggtctgaggacacggccaattattactgtgcaaagtgcggctacccgggtataacgtcttttgattactggggccaaggagtcatggtcacagtctcctca

SEQ ID NO: 286 - DNA, 321 bp, synthetic construct
  misc_feature 1..321: note = Synthetic: ZNRF3-219 (VL DNA)
  source 1..321: mol_type = other DNA
gacatccagatgacccagtctccttcattcctgtctgcatctgtgggagacagagtctctatcaactgcaaagcaagtcagaatattggcaagtacttaaactggtatcagcaagagcttggagaagctcccaaacgcctgatatataatacaaacaatttgcaaacaggcatcccatcaaggttcagtggcagtggatctggtacagattacacactcaccatcagcggcctgcagcctgaagattttgccacatatttctgcttgcagcataatggttttccgctcacgttcggttctgggaccaagctggagatcaaa

SEQ ID NO: 287 - protein, 120 aa, synthetic construct
  REGION 1..120: note = Synthetic: ZNRF3-219 (VH prot)
EVQLVESGGGLVQPGRSLKVSCAASGFTFSTFAMAWVRQAPKKGLEWVATITPGGSNTYYPDSVKGRFTISRDNAKSTLYLQMDSLRSEDTANYYCAKCGYPGITSFDYWGQGVMVTVSS

SEQ ID NO: 288 - protein, 107 aa, synthetic construct
  REGION 1..107: note = Synthetic: ZNRF3-219(VL prot)
DIQMTQSPSFLSASVGDRVSINCKASQNIGKYLNWYQQELGEAPKRLIYNTNNLQTGIPSRFSGSGSGTDYTLTISGLQPEDFATYFCLQHNGFPLTFGSGTKLEIK

SEQ ID NO: 289 - DNA, 354 bp, synthetic construct
  misc_feature 1..354: note = Synthetic: ZNRF3-222 (VH DNA)
  source 1..354: mol_type = other DNA
gaagtgcagctggtggagtctggtggaggcctagtgcagccgggaaggtccctgaaactctcctgtgcagtctcaggattcaccttcagtaactatggcatggcctgggtccgccagactccaacgaaggggctggagtgggtcgcaaccattacttatgatggtagtaccacttactatcgagactccgtgaagggccgattcactatctccagagataatgcaaagagcgccctatccctgcaaatggacagtctgaggtctgaggacacggccacttatcactgtgcaaggcagggtttggggtacccgtttgtctactggggtcaaggcactctggtcactgtctcttcc

SEQ ID NO: 290 - DNA, 336 bp, synthetic construct
  misc_feature 1..336: note = Synthetic: ZNRF3-222 (VL DNA)
  source 1..336: mol_type = other DNA
gatgttgtgatgacacaaactccagtctccctgcctgtcagccttggaggtcaagcctctatctcttgccggacaagtcagagcctggtacatagtaatggaaacacctacttgcattggtacctgcagaagccaggccagtctccacagctcctcatctatcgggtttccaacagattctctggggtgccagacaggttcagtggcagtgggtcagggacagatttcaccctcaagatcagtagaatagagcctgaggacttgggagattattactgcttacaaagtacacattttccgtacacgtttggagctgggaccaagctggaactgaaa

SEQ ID NO: 291 - protein, 118 aa, synthetic construct
  REGION 1..118: note = Synthetic: ZNRF3-222 (VH prot)
EVQLVESGGGLVQPGRSLKLSCAVSGFTFSNYGMAWVRQTPTKGLEWVATITYDGSTTYYRDSVKGRFTISRDNAKSALSLQMDSLRSEDTATYHCARQGLGYPFVYWGQGTLVTVSS

SEQ ID NO: 292 - protein, 112 aa, synthetic construct
  REGION 1..112: note = Synthetic: ZNRF3-222(VL prot)
DVVMTQTPVSLPVSLGGQASISCRTSQSLVHSNGNTYLHWYLQKPGQSPQLLIYRVSNRFSGVPDRFSGSGSGTDFTLKISRIEPEDLGDYYCLQSTHFPYTFGAGTKLELK

SEQ ID NO: 293 - DNA, 348 bp, synthetic construct
  misc_feature 1..348: note = Synthetic: ZNRF3-223 (VH DNA)
  source 1..348: mol_type = other DNA
gaggtacagctggtggagtctggaggaggcttagtgcagcctggagagtccctgaaactctcctgttcagcctctggattcacattcagtaactatggcatgcactggatacgccaagttccaggaaaggggctagattgggttgcatacattactaatagcggcggttcactctatgcagccgctgtgaagggccggttcaccatctccagagacaatgcaaagaacaccctgtacctggaactcaccagtctgaagtctgaggacactgccatctatcactgtgcaagaggttatagttacccgggaccttactggggccgaggcactctggtcacggtctcttca

SEQ ID NO: 294 - DNA, 321 bp, synthetic construct
  misc_feature 1..321: note = Synthetic: ZNRF3-223 (VL DNA)
  source 1..321: mol_type = other DNA
gatattcaagtgactcaatctccatcctccctcttggcatctctaggagagagagtcactatcacatgccagacaggtcagagcattaacaattacctaaactggtatcagcagaggccaggacaagctcctctactcttgatctattttgcaaccagtttgcagactggcgtgccatcaaggttcagtggccaatattctgggagaagtttcactctaaccatcactagcctggagccagaagatattgcaaattatttttgtctgcaacattacagtcctccgtacacgtttggagctgggaccaagctggaactgaga

SEQ ID NO: 295 - protein, 116 aa, synthetic construct
  REGION 1..116: note = Synthetic: ZNRF3-223 (VH prot)
EVQLVESGGGLVQPGESLKLSCSASGFTFSNYGMHWIRQVPGKGLDWVAYITNSGGSLYAAAVKGRFTISRDNAKNTLYLELTSLKSEDTAIYHCARGYSYPGPYWGRGTLVTVSS

SEQ ID NO: 296 - protein, 107 aa, synthetic construct
  REGION 1..107: note = Synthetic: ZNRF3-223(VL prot)
DIQVTQSPSSLLASLGERVTITCQTGQSINNYLNWYQQRPGQAPLLLIYFATSLQTGVPSRFSGQYSGRSFTLTITSLEPEDIANYFCLQHYSPPYTFGAGTKLELR

SEQ ID NO: 297 - DNA, 348 bp, synthetic construct
  misc_feature 1..348: note = Synthetic: ZNRF3-231 (VH DNA)
  source 1..348: mol_type = other DNA
gaggtacagctggtggagtctggaggaggcttagtgcagcctggagagtccctgaaactctcctgttcagcctctggattcacattcagtaactatggaatgcactggatacgccaagttccaggaaagggactagattgggttgcatacattagtaatagcggcggttcactctatgcagacgctgtgaagggccggttcaccatctccagagacaatgcaaagaacaccctatacctggaactcaacagtctgaagtctgaggacactgccatctatcactgtgcaagaggttatagttacccgggaccttactggggccaaggcactctggtcacggtctcttca

SEQ ID NO: 298 - DNA, 321 bp, synthetic construct
  misc_feature 1..321: note = Synthetic: ZNRF3-231 (VL DNA)
  source 1..321: mol_type = other DNA
gatattcaagtgactcaatctccatcctccctcttggcatctctaggagagagagtcactatcacatgccagacaggtcagagtattaacaattacctgaactggtatcagcagaggccaggacaagctcctctactcttgatctattttgcaaccagtttgcagactggcgtgccatcaaggttcagtggccaatattctgggagaagtttcactctaaccatcactagcctggagccagaagatattgcaaattatttttgtctgcaacattacagtcctccgtacacgtttggagctgggaccaagctggaactgaga

SEQ ID NO: 299 - protein, 116 aa, synthetic construct
  REGION 1..116: note = Synthetic: ZNRF3-231 (VH prot)
EVQLVESGGGLVQPGESLKLSCSASGFTFSNYGMHWIRQVPGKGLDWVAYISNSGGSLYADAVKGRFTISRDNAKNTLYLELNSLKSEDTAIYHCARGYSYPGPYWGQGTLVTVSS

SEQ ID NO: 300 - protein, 107 aa, synthetic construct; identical to SEQ ID NO: 296
  REGION 1..107: note = Synthetic: ZNRF3-231(VL prot)

SEQ ID NO: 301 - DNA, 360 bp, synthetic construct
  misc_feature 1..360: note = Synthetic: ZNRF3-237 (VH DNA)
  source 1..360: mol_type = other DNA
cagatccagttggtgcagtctggacctgagctgaagaagcctggagagtcagtgaagatctcctgcaaggcttctgggtataccttcacagactatgcaatgcactgggtgaaacaggttccaggaaagggcttgaagtggatgggctggatcaacacctatactgggaagccaacatatgctgatgacttcaaaggacggtttgtcttctctttggaagcctctgccagcactgtaaatttgcagatcagcaacctcaaaaatgaggacacggctaatgttttctgtgcaagaggggggttattatcacgtggttggtttgcttactggggccaaggcactctggtcactgtctcttca

SEQ ID NO: 302 - DNA, 336 bp, synthetic construct
  misc_feature 1..336: note = Synthetic: ZNRF3-237 (VL DNA)
  source 1..336: mol_type = other DNA
gatattgtgatgacacaaactccagtctccctgtctgtcagccttggaggtcaagtctctatctcttgccggtctagtcagagctttgttcatagtgatggaaatagctatttgagttggtacctgcagaagcccggccaatctccacagctcctcatttatgaggtttccaaccgattatctggggtcccagataggttcagtggtagtggttcagggacagatttcaccctcaagatcagcagagtagagcatgatgatttgggagtttattactgcggccaagcttcaaaaattccgctcacgttcggttctgggaccaagctggagatcaaa

SEQ ID NO: 303 - protein, 120 aa, synthetic construct
  REGION 1..120: note = Synthetic: ZNRF3-237 (VH prot)
QIQLVQSGPELKKPGESVKISCKASGYTFTDYAMHWVKQVPGKGLKWMGWINTYTGKPTYADDFKGRFVFSLEASASTVNLQISNLKNEDTANVFCARGGLLSRGWFAYWGQGTLVTVSS

SEQ ID NO: 304 - protein, 112 aa, synthetic construct
  REGION 1..112: note = Synthetic: ZNRF3-237(VL prot)
DIVMTQTPVSLSVSLGGQVSISCRSSQSFVHSDGNSYLSWYLQKPGQSPQLLIYEVSNRLSGVPDRFSGSGSGTDFTLKISRVEHDDLGVYYCGQASKIPLTFGSGTKLEIK

SEQ ID NO: 305 - DNA, 348 bp, synthetic construct
  misc_feature 1..348: note = Synthetic: ZNRF3-244 (VH DNA)
  source 1..348: mol_type = other DNA
gaggtacagctggtggagtctggaggaggcttagtgcagcctggaaagtccctgaaactctcctgttcagcctctggattcacattcagtagctatggcatgcactggatacgccaaactccaggaaagggactagattgggttgcatacattagtactaacagcggtacagtctatgcagacgctgtgaagggccggttcaccatctccagagacaatgcaaagaacaccctgtacctgcaactcaacagtctgcagtctgaagacactgccatctattactgtgcaagaggttataactacccgggagcttactggggccaaggcactctggtcactgtctcttca

SEQ ID NO: 306 - DNA, 321 bp, synthetic construct
  misc_feature 1..321: note = Synthetic: ZNRF3-244 (VL DNA)
  source 1..321: mol_type = other DNA
gatattcaagtgactcaatctccatcctccctcttggcatctctaggagagagagtcactatcacatgccagacaagtcagagcattagcaataacctaaactggtatcagcagaaaccaggacaagctcctatgctcttgatctattatgcaaccagtttgcagactggcatgccatcaaggttcagtggccaatattctgggagaagtttcactctaaccatcactagcctggagccagaagatattgcaaattatttttgtctgcaacattacaatcctccgtacacgtttggagctgggaccaagctggaactgaaa

SEQ ID NO: 307 - protein, 116 aa, synthetic construct
  REGION 1..116: note = Synthetic: ZNRF3-244 (VH prot)
EVQLVESGGGLVQPGKSLKLSCSASGFTFSSYGMHWIRQTPGKGLDWVAYISTNSGTVYADAVKGRFTISRDNAKNTLYLQLNSLQSEDTAIYYCARGYNYPGAYWGQGTLVTVSS

SEQ ID NO: 308 - protein, 107 aa, synthetic construct
  REGION 1..107: note = Synthetic: ZNRF3-244(VL prot)
DIQVTQSPSSLLASLGERVTITCQTSQSISNNLNWYQQKPGQAPMLLIYYATSLQTGMPSRFSGQYSGRSFTLTITSLEPEDIANYFCLQHYNPPYTFGAGTKLELK

SEQ ID NO: 309 - DNA, 348 bp, synthetic construct
  misc_feature 1..348: note = Synthetic: ZNRF3-247 (VH DNA)
  source 1..348: mol_type = other DNA
gaggtgcagctggtggggtctggtgaaggcttagtacagcctggaagttccatgaaactctcatgtgtagcctctggattcactttcagtaactctggcatgaactggattcgacaggctccaaagaaggggctggaatggatagcactgatttattatactaacacatactatgcagactctgtgaagggtcgattcaccatctccagagacaattctaagaacaccctgtacctggaaatgaacagtctgagatctgaggacacagccatgtattactgtgcaaaaaggggtctaactgggagcgttgattactggggccaaggagtcatggtcacagtctcctca

SEQ ID NO: 310 - DNA, 321 bp, synthetic construct
  misc_feature 1..321: note = Synthetic: ZNRF3-247 (VL DNA)
  source 1..321: mol_type = other DNA
gacatccagatgacccagtctccttcattcctgtctgcatctgtgggagacagagtcactatcaactgcaaagcaagtcagaatattaacaggtacttaaactggtatcagcagaagcttggagaagctcccaaacgcctaatatatgatacaagcaatttgcaaacgggcattccatcaaggttcagtggcagtgggtctggtacagatttcacactcaccatcagcagcctgcagcctgaagattttgccacatatttctgcttgcagcatgatagttttcccaacacgtttggaactgggaccaagctggagctgaaa

SEQ ID NO: 311 - protein, 116 aa, synthetic construct
  REGION 1..116: note = Synthetic: ZNRF3-247 (VH prot)
EVQLVGSGEGLVQPGSSMKLSCVASGFTFSNSGMNWIRQAPKKGLEWIALIYYTNTYYADSVKGRFTISRDNSKNTLYLEMNSLRSEDTAMYYCAKRGLTGSVDYWGQGVMVTVSS

SEQ ID NO: 312 - protein, 107 aa, synthetic construct
  REGION 1..107: note = Synthetic: ZNRF3-247(VL prot)
DIQMTQSPSFLSASVGDRVTINCKASQNINRYLNWYQQKLGEAPKRLIYDTSNLQTGIPSRFSGSGSGTDFTLTISSLQPEDFATYFCLQHDSFPNTFGTGTKLELK

SEQ ID NO: 313 - DNA, 363 bp, synthetic construct
  misc_feature 1..363: note = Synthetic: ZNRF3-253 (VH DNA)
  source 1..363: mol_type = other DNA
gaggtgcagcttgtagagtctgggggcggtttagtgcagcctgaaaggtccatgaaactctcctgtgcagcctcaggattcactttcagtaactatggcatggcctgggtccgccaggctccaacgaaggggctggagtgggttgcaaccattaattctgatggtagtggcacttactatcgaggctccgtgaagggccgattcactatctccagagataatgcaaaaagcaccctatatctacaaatgaacagtctgaggtctgacgacacggccacttattactgtacaagaggtcggatgctacggatcattagcttctttgattactggggccaaggagtcatggtcacagtctcttca

SEQ ID NO: 314 - DNA, 321 bp, synthetic construct
  misc_feature 1..321: note = Synthetic: ZNRF3-253 (VL DNA)
  source 1..321: mol_type = other DNA
gacatccagatgacacagtctccagcttccctgtcagcatctcttggagaaactgtctccatcgaatgtctagcaagtgaggacattgccaatgatttagcgtggtatcaacagaagtcagggaaatctcctcaggtcctgatctatgctgcaagtagggtgcaagacggggtcccatcacggttcagtggcagtggatctggcacacggtattctctcaagatcaccgacatgcaacctgaagatgtggcagattatttctgtcaacagagttacggctttccgctcacgttcggttctgggaccaagctagagatcaaa

SEQ ID NO: 315 - protein, 121 aa, synthetic construct
  REGION 1..121: note = Synthetic: ZNRF3-253 (VH prot)
EVQLVESGGGLVQPERSMKLSCAASGFTFSNYGMAWVRQAPTKGLEWVATINSDGSGTYYRGSVKGRFTISRDNAKSTLYLQMNSLRSDDTATYYCTRGRMLRIISFFDYWGQGVMVTVSS

SEQ ID NO: 316 - protein, 107 aa, synthetic construct
  REGION 1..107: note = Synthetic: ZNRF3-253(VL prot)
DIQMTQSPASLSASLGETVSIECLASEDIANDLAWYQQKSGKSPQVLIYAASRVQDGVPSRFSGSGSGTRYSLKITDMQPEDVADYFCQQSYGFPLTFGSGTKLEIK

SEQ ID NO: 317 - DNA, 363 bp, synthetic construct
  misc_feature 1..363: note = Synthetic: ZNRF3-254 (VH DNA)
  source 1..363: mol_type = other DNA
gaggtgcaactggtagagtctgggggcggtctagtgcagcctgaaaggtccatgaaactctcctgtgcagcctcaggattcactttcaataactatggcatggcctgggtccgccagactccaacgaagggactggaatgggttgcaaccattaattctgatggtagtggcactcactatcgaggctccgtgaagggccgattcactatctccagagataatgcaaaaggcaccctatatctacaaatgcacaatctgaggtctgacgacacggccacttattactgtgcacgaggtcggatacttcggatcattacctactttgattcctggggccaaggagtcgtggtcacagtctcctca

SEQ ID NO: 318 - DNA, 321 bp, synthetic construct
  misc_feature 1..321: note = Synthetic: ZNRF3-254 (VL DNA)
  source 1..321: mol_type = other DNA
gacatccagatgacacagtctccagcttccctgtcagcatctcttggagaaagtgtctccatcgaatgtctagcaagtgaggacattgccaatgatttagcgtggtatcaacagaagtcagggagatctcctcaggtcctgatccatgctgcaagtagggtgcaagacggggtcccatcacggttcagtggcagtggatctggcacacggtactctctcaagatcaccgacatgcaacctgaagatgtagcagattatttctgtcaacagagttacggctttccgctcacgttcggttctgggaccaagctagagatcaaa

SEQ ID NO: 319 - protein, 121 aa, synthetic construct
  REGION 1..121: note = Synthetic: ZNRF3-254 (VH prot)
EVQLVESGGGLVQPERSMKLSCAASGFTFNNYGMAWVRQTPTKGLEWVATINSDGSGTHYRGSVKGRFTISRDNAKGTLYLQMHNLRSDDTATYYCARGRILRIITYFDSWGQGVVVTVSS

SEQ ID NO: 320 - protein, 107 aa, synthetic construct
  REGION 1..107: note = Synthetic: ZNRF3-254(VL prot)
DIQMTQSPASLSASLGESVSIECLASEDIANDLAWYQQKSGRSPQVLIHAASRVQDGVPSRFSGSGSGTRYSLKITDMQPEDVADYFCQQSYGFPLTFGSGTKLEIK

SEQ ID NO: 321 - DNA, 342 bp, synthetic construct
  misc_feature 1..342: note = Synthetic: ZNRF3-255 (VH DNA)
  source 1..342: mol_type = other DNA
gaggtgcagctggtggagtcggggggaggcttagtgcagcctggaaggtccctgaaactctcctgcgcagcctcaggattcactttcagtagctttacaatggcctgggtccgccaggctccaaagaagggtctggagtgggtcgcatccattagttctggtggtggtggcacttactatccagattccgtgaagggcagatttactatctccagagataatgcaaaaagcaccctatacctgcaaatggacagtctgagatctgaggacacggccagttactattgtgcaaccggggggtttgcttactggggccagggcactctggtcattgtctcttca

SEQ ID NO: 322 - DNA, 321 bp, synthetic construct
  misc_feature 1..321: note = Synthetic: ZNRF3-255 (VL DNA)
  source 1..321: mol_type = other DNA
gacatccagatgacccagtctccatcttccctgcctgcatctctgggagacagagtcactattacttgccgggcaagtcaagacattggaagttatttaagatggttccagcagaaaccggggaagtctcctaggcttatgatttatggtgcaaccagcttggcaaatggggtcccatcaaggttcagtggcagtaggtctgggtcagattattctctgaccatcaacaacttggagtctgaagatatggctatttattactgtctgcagcataatgagtttccgtacacgtttggaactgggaccaagctggaactgaaa

SEQ ID NO: 323 - protein, 114 aa, synthetic construct
  REGION 1..114: note = Synthetic: ZNRF3-255 (VH prot)
EVQLVESGGGLVQPGRSLKLSCAASGFTFSSFTMAWVRQAPKKGLEWVASISSGGGGTYYPDSVKGRFTISRDNAKSTLYLQMDSLRSEDTASYYCATGGFAYWGQGTLVIVSS

SEQ ID NO: 324 - protein, 107 aa, synthetic construct
  REGION 1..107: note = Synthetic: ZNRF3-255(VL prot)
DIQMTQSPSSLPASLGDRVTITCRASQDIGSYLRWFQQKPGKSPRLMIYGATSLANGVPSRFSGSRSGSDYSLTINNLESEDMAIYYCLQHNEFPYTFGTGTKLELK

SEQ ID NO: 325 - DNA, 363 bp, synthetic construct
  misc_feature 1..363: note = Synthetic: ZNRF3-265 (VH DNA)
  source 1..363: mol_type = other DNA
gaggtgcagttggtagagtctgggggcggtttagtgcagcctggaaggtccatgaaactctcctgtgtagcctcaggattcagtttcagtaaatatggcatggcctgggtccgccaggctccgacgaaggctttggagtgggttgcaatcattagtcaggatggtagtagaatttactatggaggctccgtgaagggccgattcactctctccagagataatgaaaaaagcaccctacacctgcacatgaacagtctgaggtctgaggacacggccacttattattgtgcaagaggaagtatactacggatgataactttctttgattattggggccaaggagtcacggtcacagtctcctca

SEQ ID NO: 326 - DNA, 321 bp, synthetic construct
  misc_feature 1..321: note = Synthetic: ZNRF3-265 (VL DNA)
  source 1..321: mol_type = other DNA
gacatccagatgactcagtctccagcttccctgtctgcatctcttggagaaactgtctccatcgaatgtctagcaagtgagggcatttccaatgatttagcgtggtatcagcagaagtcagggaaatctcctcggctcctgatctatgctgcaagtaggttgcaagacggggtcccatcacggttcagtggcagtggatctgacacacggttttctctcaagatcagcggcatgcaccctgaagatgaagcagattatttctgtcaacagagttacggttatccgctcacgttcggttctgggaccaagctggagatcaaa

SEQ ID NO: 327 - protein, 121 aa, synthetic construct
  REGION 1..121: note = Synthetic: ZNRF3-265 (VH prot)
EVQLVESGGGLVQPGRSMKLSCVASGFSFSKYGMAWVRQAPTKALEWVAIISQDGSRIYYGGSVKGRFTLSRDNEKSTLHLHMNSLRSEDTATYYCARGSILRMITFFDYWGQGVTVTVSS

SEQ ID NO: 328 - protein, 107 aa, synthetic construct
  REGION 1..107: note = Synthetic: ZNRF3-265(VL prot)
DIQMTQSPASLSASLGETVSIECLASEGISNDLAWYQQKSGKSPRLLIYAASRLQDGVPSRFSGSGSDTRFSLKISGMHPEDEADYFCQQSYGYPLTFGSGTKLEIK

SEQ ID NO: 329 - DNA, 354 bp, synthetic construct
  misc_feature 1..354: note = Synthetic: ZNRF3-269 (VH DNA)
  source 1..354: mol_type = other DNA
gagatacaacttcaggagtcaggacctggccttgtgaaaccttcacagtcactctccctcacttgttctgtcactgatgacaccattaccagtggttatgattggagctggatccggaagttcccgggaaataagatggagtggatgggggacataagcagcagcgggcacacgaactacaacccttcgctccagattcgcatctccataactagagacacatccaagaatcagttctttctgcagttgaactctgtaacaactgaggattcagccacctattattgtgctagaggcggttttttatttccttttactttctggggccaaggcactctggtcactgtctcttca

SEQ ID NO: 330 - DNA, 321 bp, synthetic construct
  misc_feature 1..321: note = Synthetic: ZNRF3-269 (VL DNA)
  source 1..321: mol_type = other DNA
gacatccagatgacccagtctccatcctccctgcctgcatctctgggagagagagtcaccatcagttgcagagcaagtcaggttcttaacaataatttaaactggtatcagcagaaaccagatggaacgattaaacccctactctactacacatccaatttacaatctggtgtcccatcaaggttcagtggcagtgggtctgggacagattattctctcaccatcaccagccttgaccctgaagattttgcaatatattactgccaacaggatgctagttttcctccgacgttcggtggcggcaccaagctggaattgaaa

SEQ ID NO: 331 - protein, 118 aa, synthetic construct
  REGION 1..118: note = Synthetic: ZNRF3-269 (VH prot)
EIQLQESGPGLVKPSQSLSLTCSVTDDTITSGYDWSWIRKFPGNKMEWMGDISSSGHTNYNPSLQIRISITRDTSKNQFFLQLNSVTTEDSATYYCARGGFLFPFTFWGQGTLVTVSS

SEQ ID NO: 332 - protein, 107 aa, synthetic construct
  REGION 1..107: note = Synthetic: ZNRF3-269(VL prot)
DIQMTQSPSSLPASLGERVTISCRASQVLNNNLNWYQQKPDGTIKPLLYYTSNLQSGVPSRFSGSGSGTDYSLTITSLDPEDFAIYYCQQDASFPPTFGGGTKLELK

SEQ ID NO: 333 - DNA, 348 bp, synthetic construct
  misc_feature 1..348: note = Synthetic: ZNRF3-270 (VH DNA)
  source 1..348: mol_type = other DNA
gaggtacagctggtggagtctggaggaggcttagtgcagcctggagagtccctgaaactctcctgttcagcctctggattcacattcagtaactatggcatgcactggatacgccaagttccaggaaaggggctagattgggttgcatacattactaatagcggcggttcactctatgcagccgctgtgaagggccggttcaccatctccagagacaatgcaaagaacaccctgtacctggaactcaccagtctgaagcctgaggacactgccatctatcactgtgcaagaggttacagttatccgggaccttactggggccgaggcactctggtcacggtctcttca

SEQ ID NO: 334 - DNA, 321 bp, synthetic construct
  misc_feature 1..321: note = Synthetic: ZNRF3-270 (VL DNA)
  source 1..321: mol_type = other DNA
gatattcaagtgactcaatctccatcctccctcttggcatctctaggagagagagtcactatcacatgccagacaggtcagagcattaacaattacctaaactggtatcagcagaggccaggacaagctcctctactcttgatctattttgcaaccagtttgcagactggcgtgccatcaaggttcagtggccaatattctgggagaagtttcactctaaccatcactagcctggagccagaagatattgcaaattatttttgtctgcagcattacagtcctccgtacacgtttggagctgggaccaagctggaactgaga

SEQ ID NO: 335 - protein, 116 aa, synthetic construct
  REGION 1..116: note = Synthetic: ZNRF3-270 (VH prot)
EVQLVESGGGLVQPGESLKLSCSASGFTFSNYGMHWIRQVPGKGLDWVAYITNSGGSLYAAAVKGRFTISRDNAKNTLYLELTSLKPEDTAIYHCARGYSYPGPYWGRGTLVTVSS

SEQ ID NO: 336 - protein, 107 aa, synthetic construct; identical to SEQ ID NO: 296
  REGION 1..107: note = Synthetic: ZNRF3-270(VL prot)

SEQ ID NO: 337 - DNA, 357 bp, synthetic construct
  misc_feature 1..357: note = Synthetic: ZNRF3-275 (VH DNA)
  source 1..357: mol_type = other DNA
caggtgcagctgaaggagtcaggacctggccttgtgcagccctcacagaccctgtccctcacctgcactgtctctgggttctcatttaactactataatgttcactggcttcgacagcctccaggaaagggtctggagtggttgggagtaatctggagtggtggaaacagagattacaattcagctctcaaaccccgactgcgcatcagcagggacacctccaagacccaagttttcttaacattgaacagtctgcagactgaggacacaggcatttactattgtaccagaatgtggggctatgggtacaacccctttgatcactggggccaaggagtcatggtcacagtctcctca

SEQ ID NO: 338 - DNA, 321 bp, synthetic construct
  misc_feature 1..321: note = Synthetic: ZNRF3-275 (VL DNA)
  source 1..321: mol_type = other DNA
gacatccagatgactcagtctccgtcatttctgtctgcatctcttggagacaacatcaccatcacttgccatgccagtcaggacatcaagggttggttagcctggtaccaacaaaagtcagggaatgctcctgaactgttgatttataaggcatctagcctgcaatcaggggttccatcaagattcagtggcggcggatctggaacagaatatgttctcactatcagcaacctacagcctgacgatattgccacttattactgtcagctttatcacagctttccattcacgtttggctcagggacgaagttggaaacaaaa

SEQ ID NO: 339 - protein, 119 aa, synthetic construct
  REGION 1..119: note = Synthetic: ZNRF3-275 (VH prot)
QVQLKESGPGLVQPSQTLSLTCTVSGFSFNYYNVHWLRQPPGKGLEWLGVIWSGGNRDYNSALKPRLRISRDTSKTQVFLTLNSLQTEDTGIYYCTRMWGYGYNPFDHWGQGVMVTVSS

SEQ ID NO: 340 - protein, 107 aa, synthetic construct
  REGION 1..107: note = Synthetic: ZNRF3-275(VL prot)
DIQMTQSPSFLSASLGDNITITCHASQDIKGWLAWYQQKSGNAPELLIYKASSLQSGVPSRFSGGGSGTEYVLTISNLQPDDIATYYCQLYHSFPFTFGSGTKLETK

SEQ ID NO: 341 - DNA, 360 bp, synthetic construct
  misc_feature 1..360: note = Synthetic: ZNRF3-279 (VH DNA)
  source 1..360: mol_type = other DNA
caggtccagctgcagcagtctggagctgagctggtgaagcctgggacttctgtgaggctgtcctgcaaggcttctggctacacctttactcacaaccatatgaactggataaagcagacgactggacagggccttgagtggattggaattattaatccgggaagtggaggtactaattacaatgtgaggttcaagggcaaggccacattgactgtagacacatcctccagcacagccttcatgcaactcagcagcctgacacctgaggactctgcggtctattactgtgcaagaaaccttattactatagcaggcccgtttgcttactggggccaaggcactctggtcactgtctcttca

SEQ ID NO: 342 - DNA, 315 bp, synthetic construct
  misc_feature 1..315: note = Synthetic: ZNRF3-279 (VL DNA)
  source 1..315: mol_type = other DNA
gacactgtactgacccagtctcctgctttgactgtgtctccaggagagagggtttccatctcctgtagggccagtgaaggggtcagttcatatatacactggtaccaacagaaaccaggacagcagcccaaactcctcatctataaagcatcaaacctagcatctggggtccctgccaggttcagtggcagtgggtctgggacagacttcaccctcaccattgatcctgtggaggctgatgacactgcaacctatttctgtcagcagagttggattgatcacacgtttggaactgggaccaagctggaactgaaa

SEQ ID NO: 343 - protein, 120 aa, synthetic construct
  REGION 1..120: note = Synthetic: ZNRF3-279 (VH prot)
QVQLQQSGAELVKPGTSVRLSCKASGYTFTHNHMNWIKQTTGQGLEWIGIINPGSGGTNYNVRFKGKATLTVDTSSSTAFMQLSSLTPEDSAVYYCARNLITIAGPFAYWGQGTLVTVSS

SEQ ID NO: 344 - protein, 105 aa, synthetic construct
  REGION 1..105: note = Synthetic: ZNRF3-279(VL prot)
DTVLTQSPALTVSPGERVSISCRASEGVSSYIHWYQQKPGQQPKLLIYKASNLASGVPARFSGSGSGTDFTLTIDPVEADDTATYFCQQSWIDHTFGTGTKLELK

SEQ ID NO: 345 - DNA, 354 bp, synthetic construct
  misc_feature 1..354: note = Synthetic: ZNRF3-287 (VH DNA)
  source 1..354: mol_type = other DNA
gaggtgcagttggtggggtctggtggaggcttggtgcagcctggaaggtctctgaaaatctcctgtgcagcctcaggattcactttcagttactatggcatggcctgggtccgccaggctccaacgagggggctggagtgggtcgcaaccattacttataatggttctaagtcttactatcgagactccgtgaagggccgattcactatctccagagagtatgcaaaaagcaccctatacctccaaatggacgatctgacgtctgaagacacggccacttatttctgtgcaagacggactacgggtatgctctttgattactggggccaaggagtcttggtcacagtctcctca

SEQ ID NO: 346 - DNA, 330 bp, synthetic construct
  misc_feature 1..330: note = Synthetic: ZNRF3-287 (VL DNA)
  source 1..330: mol_type = other DNA
caggctgtccttactcagccaaactctgtgtctatgtctctaggaagcacagtcaagctgtcttgcacactcaactctggtaacatagaaaacaactttgtgcactggtaccaacaatatgagggaagatctcccaccactatgatttataatgatgataagagaccggatggtgtccctgacaggttctctggctccattgacagctcttccaactcagccttcctgacaatcaataatgtggaaattggagatgaagctatctacttctgtcattcttttgttagtagtattcatattttcggcggtggaaccaagctcactgtccta

SEQ ID NO: 347 - protein, 118 aa, synthetic construct
  REGION 1..118: note = Synthetic: ZNRF3-287 (VH prot)
EVQLVGSGGGLVQPGRSLKISCAASGFTFSYYGMAWVRQAPTRGLEWVATITYNGSKSYYRDSVKGRFTISREYAKSTLYLQMDDLTSEDTATYFCARRTTGMLFDYWGQGVLVTVSS

SEQ ID NO: 348 - protein, 110 aa, synthetic construct
  REGION 1..110: note = Synthetic: ZNRF3-287(VL prot)
QAVLTQPNSVSMSLGSTVKLSCTLNSGNIENNFVHWYQQYEGRSPTTMIYNDDKRPDGVPDRFSGSIDSSSNSAFLTINNVEIGDEAIYFCHSFVSSIHIFGGGTKLTVL

SEQ ID NO: 349 - DNA, 351 bp, synthetic construct
  misc_feature 1..351: note = Synthetic: ZNRF3-296 (VH DNA)
  source 1..351: mol_type = other DNA
gaggtacagctggtggagtctggaggaggcttagtgcagcctggaaagtccctgagactctcctgttcagcctctggattcacattcagtaactatggcctgcactggatacgccaagctccaggaaagggactagattggcttggatatagtagtagtaacagcggtacagtctatggagacgctgtgaagggccgggtcaccatctccagagacaatgcaaagaacaccctgtacctgcaactcaacagtctgaagtctgaagacactgccatctattattgtgcaagaggctatagcagctccccgtttgcttactggggccaaggcactctggtcactgtctcttca

SEQ ID NO: 350 - DNA, 330 bp, synthetic construct
  misc_feature 1..330: note = Synthetic: ZNRF3-296 (VL DNA)
  source 1..330: mol_type = other DNA
gacattgtgctgacccagtctcctgctttggctgtgtctctaggccagagagtcaccatctcatgcaaagccagccagaatgtcgatggttttggcattggttatatgcactggtaccaacagaaaccaggacagaaacccaaactcctcatctttgcaggatccaacttagcatctgggatccctgccaggttcagtggcagtgggtctgggacagacttcaccctcaccatcgatcctgtggagactgatgatattgcaacctattactgtcagcagaataaagattttcccctcacgttcggttctgggtccaggctggagatcaca

SEQ ID NO: 351 - protein, 117 aa, synthetic construct
  REGION 1..117: note = Synthetic: ZNRF3-296 (VH prot)
EVQLVESGGGLVQPGKSLRLSCSASGFTFSNYGLHWIRQAPGKGLDWLGYSSSNSGTVYGDAVKGRVTISRDNAKNTLYLQLNSLKSEDTAIYYCARGYSSSPFAYWGQGTLVTVSS

SEQ ID NO: 352 - protein, 110 aa, synthetic construct
  REGION 1..110: note = Synthetic: ZNRF3-296(VL prot)
DIVLTQSPALAVSLGQRVTISCKASQNVDGFGIGYMHWYQQKPGQKPKLLIFAGSNLASGIPARFSGSGSGTDFTLTIDPVETDDIATYYCQQNKDFPLTFGSGSRLEIT

SEQ ID NO: 353 - DNA, 360 bp, synthetic construct
  misc_feature 1..360: note = Synthetic: ZNRF3-30 (VH DNA)
  source 1..360: mol_type = other DNA
gaggtgcagctggcggagtctgggggaggcttagagcagcctggaaggtccctgaaactctcctgtgcagcctcaggattcactttcagtaactatgacatggcctgggtccgccaggctccaacgaggggtctggagtgggtcgcatccattcttactagtggtggtactacttattatcgagactccgtgaagggccgattcactgtctccagagataatgcaaaaagcaccctatacctgcaaatggacagtctgaggtctgaggacacggccacttattactgtgcaagacatggggtcggtagctacgattggtttgctgactggggccaaggcactctggtcactgtctcttca

SEQ ID NO: 354 - DNA, 327 bp, synthetic construct
  misc_feature 1..327: note = Synthetic: ZNRF3-30 (VL DNA)
  source 1..327: mol_type = other DNA
gacattgtcttgacccagtctcctgctttggctgtgtctctagggcagagggccacaatctcctgtagggccagccaaagtgtcactatatctggatataatcttatgcactggtaccaacagaaaccaggacaacaacccaaactcctcatctatcgtgcatccaacctagcatttgggatccctgccaggttcagtggcagtgggtctgggacagacttcaccctcaccatcaatcctgtgcaggctgatgatattacgacctattactgtcagcagagtaggaagtctcggacgttcggtggaggcaccagactggaattgaaa

SEQ ID NO: 355 - protein, 120 aa, synthetic construct
  REGION 1..120: note = Synthetic: ZNRF3-30 (VH prot)
EVQLAESGGGLEQPGRSLKLSCAASGFTFSNYDMAWVRQAPTRGLEWVASILTSGGTTYYRDSVKGRFTVSRDNAKSTLYLQMDSLRSEDTATYYCARHGVGSYDWFADWGQGTLVTVSS

SEQ ID NO: 356 - protein, 109 aa, synthetic construct
  REGION 1..109: note = Synthetic: ZNRF3-30(VL prot)
DIVLTQSPALAVSLGQRATISCRASQSVTISGYNLMHWYQQKPGQQPKLLIYRASNLAFGIPARFSGSGSGTDFTLTINPVQADDITTYYCQQSRKSRTFGGGTRLELK

SEQ ID NO: 357 - DNA, 363 bp, synthetic construct
  misc_feature 1..363: note = Synthetic: ZNRF3-300 (VH DNA)
  source 1..363: mol_type = other DNA
gaggtgcagctggtagagtctgggggcggtttagtacagcctgaaaggtccatgaaactctcctgtgcagcctcaggattcactttcagtaactatggcatggcctgggtccgccaggctccaacgaaggggctggagtgggttgcaaccattaattctgatggtagtggcacttactatcgaggctccgtgaagggccgattcactatctccagagataatgcaagaaacaccctatacctagaaatgaacagtctgcggtctgacgacacggccacttattactgtacaagaggtcggatactacggattattacctactttgattactggggccaaggagtcatggtcacagtctcctca

SEQ ID NO: 358 - DNA, 321 bp, synthetic construct
  misc_feature 1..321: note = Synthetic: ZNRF3-300 (VL DNA)
  source 1..321: mol_type = other DNA
gacatccagatgacacagtctccagcttccctgtcagcatctgttggagaaaccgtctccatcgaatgtctagcaagtgaggacattgccaatgatttagcgtggtatcagcagaagtcagggaaatctcctcaggtcctgatctatgctgcaagtagggtgcaagacggggtcccatcacggttcagtggcagtggatctggcacacgatattctctcaggatcagcgacatgcaacctgaagatgtagcagattatttctgtcaacagagttacagctttccgctcacgttcggatctgggaccaagctggagatcaaa

SEQ ID NO: 359 - protein, 121 aa, synthetic construct
  REGION 1..121: note = Synthetic: ZNRF3-300 (VH prot)
EVQLVESGGGLVQPERSMKLSCAASGFTFSNYGMAWVRQAPTKGLEWVATINSDGSGTYYRGSVKGRFTISRDNARNTLYLEMNSLRSDDTATYYCTRGRILRIITYFDYWGQGVMVTVSS

SEQ ID NO: 360 - protein, 107 aa, synthetic construct
  REGION 1..107: note = Synthetic: ZNRF3-300(VL prot)
DIQMTQSPASLSASVGETVSIECLASEDIANDLAWYQQKSGKSPQVLIYAASRVQDGVPSRFSGSGSGTRYSLRISDMQPEDVADYFCQQSYSFPLTFGSGTKLEIK

SEQ ID NO: 361 - DNA, 351 bp, synthetic construct
  misc_feature 1..351: note = Synthetic: ZNRF3-301 (VH DNA)
  source 1..351: mol_type = other DNA
caggtacagctgaaggagtcaggacctgggctggtgcagccctcacagaccctgtccctcacctgcactgtctctgggttctcactaaccagctacagtgtacactgggttcgccagcactcaggaaagagtctggaatggatgggaagaatatggagtgatggagacacatcatataattcagcgttcacatcccgactgagcatcagcagggacacctccaagagccaagttttcttaaaaatgaacagtctccaaactgaggacacaggcacttactactgtgacagatcgggtgggagccctttctttgattattggggccaaggagtcatggtcacagtctcctca

SEQ ID NO: 362 - DNA, 330 bp, synthetic construct
  misc_feature 1..330: note = Synthetic: ZNRF3-301 (VL DNA)
  source 1..330: mol_type = other DNA
cagtttgtgcttactcagtcaaactctgtgtctacgaatctcggaagcacagtcaaactgtcttgcgagcgcagcattggtaacattggaagcaattatgtgaattggtaccagcagtatgagggaagatctcccaccactatgatttataagtttgatcagagacctgatggagttcctgacaggttctctggctccattgacagatcttccaattcagtcctcctgacaatcaataatgtgcagactgaagatgaagctgactacttctgtcagtcttacagtagtgatatgtatattttcggcggtggaaccaagctcactgtccta

SEQ ID NO: 363 - protein, 117 aa, synthetic construct
  REGION 1..117: note = Synthetic: ZNRF3-301 (VH prot)
QVQLKESGPGLVQPSQTLSLTCTVSGFSLTSYSVHWVRQHSGKSLEWMGRIWSDGDTSYNSAFTSRLSISRDTSKSQVFLKMNSLQTEDTGTYYCDRSGGSPFFDYWGQGVMVTVSS

SEQ ID NO: 364 - protein, 110 aa, synthetic construct
  REGION 1..110: note = Synthetic: ZNRF3-301(VL prot)
QFVLTQSNSVSTNLGSTVKLSCERSIGNIGSNYVNWYQQYEGRSPTTMIYKFDQRPDGVPDRFSGSIDRSSNSVLLTINNVQTEDEADYFCQSYSSDMYIFGGGTKLTVL

SEQ ID NO: 365 - DNA, 342 bp, synthetic construct
  misc_feature 1..342: note = Synthetic: ZNRF3-305 (VH DNA)
  source 1..342: mol_type = other DNA
gaggtcctgctgcagcagtcaggcgctgagcttgtgagacctgggtcctcagtcaagatttcttgcaagacttctggctacacctttacagattactatttgcactgggtgaagcagaggcctgaacagggcctggtatggataggacggattaatcctataaatggaaaaactgtttgtatagagaagttcaaaaccaaggccacactgactgcagataaatcgtccaacacagcctacatggaactcagcagcctgacatctgaagacaccgcaacctatttttgtactagaggagggcttgcttactggggccaaggcactctggtcactgtctcttca

SEQ ID NO: 366 - DNA, 321 bp, synthetic construct
  misc_feature 1..321: note = Synthetic: ZNRF3-305 (VL DNA)
  source 1..321: mol_type = other DNA
gacatccagatgacccagtctccatcgtccctgcctgcatctctgggagagagagtcaccatcagttgtagagcaagtcagggtattagcaataatttaaactggtatcagcagaaaccagatggaacgattaaacccctgatctactacacatccaatttacaatctggtgtcccatcaaggttcagtggcagtgggtctgggacagattattctctcaccatcggcagccttgagcctgaagattttacaatgtattactgccaacaggatgctagttttcctcccacgtttggagctgggaccaagctggaactgaaa

SEQ ID NO: 367 - protein, 114 aa, synthetic construct
  REGION 1..114: note = Synthetic: ZNRF3-305 (VH prot)
EVLLQQSGAELVRPGSSVKISCKTSGYTFTDYYLHWVKQRPEQGLVWIGRINPINGKTVCIEKFKTKATLTADKSSNTAYMELSSLTSEDTATYFCTRGGLAYWGQGTLVTVSS

SEQ ID NO: 368 - protein, 107 aa, synthetic construct
  REGION 1..107: note = Synthetic: ZNRF3-305(VL prot)
DIQMTQSPSSLPASLGERVTISCRASQGISNNLNWYQQKPDGTIKPLIYYTSNLQSGVPSRFSGSGSGTDYSLTIGSLEPEDFTMYYCQQDASFPPTFGAGTKLELK

SEQ ID NO: 369 - DNA, 357 bp, synthetic construct
  misc_feature 1..357: note = Synthetic: ZNRF3-312 (VH DNA)
  source 1..357: mol_type = other DNA
caggtacaactgcagcagtctggagctgagttggtgaagcctgggtcttcagtgaagatgtcctgcaaggcttctggctacagtttcaccacctactatatctattggataaatcagaggcctggacagggccttgagtggattgggcgtattggtcttggaagtggagatactgattacaatgagaaattcaagggcaaggccacatttactgtagacaaattttccaacacagcctacctgcaactcagcagcctgacacctgaggacactgcggtctattactgtgcaagatgggactacgggtatagggggtttgcttactggggccagggcactctggtcactgtctcttca

SEQ ID NO: 370 - DNA, 336 bp, synthetic construct
  misc_feature 1..336: note = Synthetic: ZNRF3-312 (VL DNA)
  source 1..336: mol_type = other DNA
gatgttgtgatgacacaaactccagtctccctgtctgtcagccttggaggtcaagtctctatctcttgccggtctagtcagagctttgtacacagtgatggaaatacctatttgaattggtacctgcagacgcccggccaatctccacaactcctcatctataaggtttccaaccgattatctggggtcccagataggttcagtggtagtggttcagggacatatttcaccctcaagatcagcagagtcgagcatgatgatttgggagtttattactgcggccaggcttcaaaaattccgctcacgttcggttccgggaccaagctggagatcaaa

SEQ ID NO: 371 - protein, 119 aa, synthetic construct
  REGION 1..119: note = Synthetic: ZNRF3-312 (VH prot)
QVQLQQSGAELVKPGSSVKMSCKASGYSFTTYYIYWINQRPGQGLEWIGRIGLGSGDTDYNEKFKGKATFTVDKFSNTAYLQLSSLTPEDTAVYYCARWDYGYRGFAYWGQGTLVTVSS

SEQ ID NO: 372 - protein, 112 aa, synthetic construct
  REGION 1..112: note = Synthetic: ZNRF3-312(VL prot)
DVVMTQTPVSLSVSLGGQVSISCRSSQSFVHSDGNTYLNWYLQTPGQSPQLLIYKVSNRLSGVPDRFSGSGSGTYFTLKISRVEHDDLGVYYCGQASKIPLTFGSGTKLEIK

SEQ ID NO: 373 - DNA, 357 bp, synthetic construct
  misc_feature 1..357: note = Synthetic: ZNRF3-314 (VH DNA)
  source 1..357: mol_type = other DNA
caggtgcagctgaaggagtcaggacctggccttgtgcagccctcacagaccctgtccctcacctgcaatgtctctggtttctcatttaactactataatgtacactgggttcgacagcctccaggaaagggtctggagtggttgggagtaatctggagtggtggaaacacagattacaattcagctctcaaaccccgactgcgcatcagcagggacacctccaagagccaagttttcttaacattgaacagtctgcagactgaggacacaggcatttactattgtaacagaatgtggggctatgggtataacccctttgattactggggccaaggagtcatggtcacagtctcctca

SEQ ID NO: 374 - DNA, 321 bp, synthetic construct
  misc_feature 1..321: note = Synthetic: ZNRF3-314 (VL DNA)
  source 1..321: mol_type = other DNA
gacatccagatgactcagtctccgtcatttctgtctgcatctcttggaaacaacatcaccatcacttgccatgccagtcaggacatcaagggttggttagcctggtaccaacaaaagtcagggaatgctcctgaactgttgatttataagacatctagcctgcaatcaggggttccatcaagattcagtggcagcggatctggaacagaatatgttctcactatcagcaacctacagcctgacgatattgccacttattactgtcagctttatcacagctttccattcacgttcggctcagggacgaagttggaaataaaa

SEQ ID NO: 375 - protein, 119 aa, synthetic construct
  REGION 1..119: note = Synthetic: ZNRF3-314 (VH prot)
QVQLKESGPGLVQPSQTLSLTCNVSGFSFNYYNVHWVRQPPGKGLEWLGVIWSGGNTDYNSALKPRLRISRDTSKSQVFLTLNSLQTEDTGIYYCNRMWGYGYNPFDYWGQGVMVTVSS

SEQ ID NO: 376 - protein, 107 aa, synthetic construct
  REGION 1..107: note = Synthetic: ZNRF3-314(VL prot)
DIQMTQSPSFLSASLGNNITITCHASQDIKGWLAWYQQKSGNAPELLIYKTSSLQSGVPSRFSGSGSGTEYVLTISNLQPDDIATYYCQLYHSFPFTFGSGTKLEIK

SEQ ID NO: 377 - DNA, 357 bp, synthetic construct
  misc_feature 1..357: note = Synthetic: ZNRF3-322 (VH DNA)
  source 1..357: mol_type = other DNA
gaggtgcacctggtggagtctgggggaggcctagtgcagcctggaaggtctctgacactatcctgtgttgcctctggattcacattcaataactggtggatgacctggatccgccaggctccagggaaggggctggagtgggttgcatccatttctcacactggtggtagcacctcctatccagactctgtgaagggccgtttcactatctccagagataatgcaaaaagtaccctatacctgcaaatgaccagtctgaggtctgacgatacggccacttattactgtacaagatggggacccatactacggggctttgattactggggccaaggagtcatggtcacagtctcctca

SEQ ID NO: 378 - DNA, 336 bp, synthetic construct
  misc_feature 1..336: note = Synthetic: ZNRF3-322 (VL DNA)
  source 1..336: mol_type = other DNA
gatgttgtgctgacccagactccacccactttatcggctaccattggacaatccgtctccatctcttgcaggtcaagtcagagtctcttacatattaatggaaacacctatgtaaattggttggtacagaggccaggccaacctccacaacttctattttatttggtatccagactggaatctggggtccccaacaggttcagtggcagtgggtcaggaactgatttcacactcaaaatcagtggagtagaggctgaggatttgggagtttattactgcgtgcaaagtacccatgctcctccgacgttcggtggaggcaccaagctggaattgaaa

SEQ ID NO: 379 - protein, 119 aa, synthetic construct
  REGION 1..119: note = Synthetic: ZNRF3-322 (VH prot)
EVHLVESGGGLVQPGRSLTLSCVASGFTFNNWWMTWIRQAPGKGLEWVASISHTGGSTSYPDSVKGRFTISRDNAKSTLYLQMTSLRSDDTATYYCTRWGPILRGFDYWGQGVMVTVSS

SEQ ID NO: 380 - protein, 112 aa, synthetic construct
  REGION 1..112: note = Synthetic: ZNRF3-322(VL prot)
DVVLTQTPPTLSATIGQSVSISCRSSQSLLHINGNTYVNWLVQRPGQPPQLLFYLVSRLESGVPNRFSGSGSGTDFTLKISGVEAEDLGVYYCVQSTHAPPTFGGGTKLELK

SEQ ID NO: 381 - DNA, 348 bp, synthetic construct
  misc_feature 1..348: note = Synthetic: ZNRF3-329 (VH DNA)
  source 1..348: mol_type = other DNA
gaggtacagctggtggagtctggaggaggcttagtgcagcctggaaagtccctgaaactctcctgttcagcctctggattcacattcagtagccatggcatgcactggatacgccaagctccaggaaaggggctagattgggttgcatacattagtaataacagcggtacaatctattcagccgctgtgaggggccggttcaccatctccagagacaattcaaagaacaccctgttcctacaactcaacagtctgaagtctgaagacactgccatctattactgtgcaagaggttataactacccgggagcttactggggccaaggcactctggtcactgtctcttca

SEQ ID NO: 382 - DNA, 321 bp, synthetic construct
  misc_feature 1..321: note = Synthetic: ZNRF3-329 (VL DNA)
  source 1..321: mol_type = other DNA
gatattcaagtgactcaatctccatcctccctcttggcatctctaggagagagagtcactatcacatgccagacaagtcagagcattagaaattacctaaactggtatcagcagaaaccaggacaagttcccatactcttgatctattatgcaaccagtttgcagactgacatgccatcaaggttcagtggccaatattctgggagaagtttcactctcaccatcactagcctggagccagaagacattgcaaattatttttgtctgcagcattacagtgttccgtacacgtttggagctgggaccaagctggaactgaaa

SEQ ID NO: 383 - protein, 116 aa, synthetic construct
  REGION 1..116: note = Synthetic: ZNRF3-329 (VH prot)
EVQLVESGGGLVQPGKSLKLSCSASGFTFSSHGMHWIRQAPGKGLDWVAYISNNSGTIYSAAVRGRFTISRDNSKNTLFLQLNSLKSEDTAIYYCARGYNYPGAYWGQGTLVTVSS

SEQ ID NO: 384 - protein, 107 aa, synthetic construct
  REGION 1..107: note = Synthetic: ZNRF3-329(VL prot)
DIQVTQSPSSLLASLGERVTITCQTSQSIRNYLNWYQQKPGQVPILLIYYATSLQTDMPSRFSGQYSGRSFTLTITSLEPEDIANYFCLQHYSVPYTFGAGTKLELK

SEQ ID NO: 385 - DNA, 372 bp, synthetic construct
  misc_feature 1..372: note = Synthetic: ZNRF3-35 (VH DNA)
  source 1..372: mol_type = other DNA
gaggtgcagttggtggagtctgggggaggcttagtgcagcctggaaggtccatgaaactctcctgtgcagcctcaggattcactttcagtaactattacatggcctgggtccgccaggctccaaggaagggtctggagtgggtcgcatccattagtactggtggtggtaacacttactatcgagaatacgtgaggggccgattcactatctccagagataatgcaaaaagcaccctatatctgcaaatggacagtctgaggtctgaggaaacggccacttattactgtgcaagacattcggggggtataatccgggcccggggtcactactttgattactggggccaaggagtcatggtcacagtctcctca

SEQ ID NO: 386 - DNA, 321 bp, synthetic construct
  misc_feature 1..321: note = Synthetic: ZNRF3-35 (VL DNA)
  source 1..321: mol_type = other DNA
gacatccagatgacacagtctccagcttccctgtctgcatctcttggagaaattgtctccatcgaatgtctagcaagtgagggcatttccaatgatttagcgtggtatcagcagaagtcagggaaatctcctcaggtcctgatctctgctgcaagtaggttgcaagacggggtcccatcacggttcagtggcagtggatctggcacacggtattctctcaagatcagcggcatgcaacctgaagacgaagcagattatttctgtcaacagagttacacgtatccgtacacgtttggagctgggaccaagctggaactgaaa

SEQ ID NO: 387 - protein, 124 aa, synthetic construct
  REGION 1..124: note = Synthetic: ZNRF3-35 (VH prot)
EVQLVESGGGLVQPGRSMKLSCAASGFTFSNYYMAWVRQAPRKGLEWVASISTGGGNTYYREYVRGRFTISRDNAKSTLYLQMDSLRSEETATYYCARHSGGIIRARGHYFDYWGQGVMVTVSS

SEQ ID NO: 388 - protein, 107 aa, synthetic construct
  REGION 1..107: note = Synthetic: ZNRF3-35(VL prot)
DIQMTQSPASLSASLGEIVSIECLASEGISNDLAWYQQKSGKSPQVLISAASRLQDGVPSRFSGSGSGTRYSLKISGMQPEDEADYFCQQSYTYPYTFGAGTKLELK

SEQ ID NO: 389 - DNA, 363 bp, synthetic construct
  misc_feature 1..363: note = Synthetic: ZNRF3-55 (VH DNA)
  source 1..363: mol_type = other DNA
gaggtgcacctggtggagtctgggggaggcttagtgcagcctggagggtccctgaaactctcctgtgcagcctcaggattcactttcagtaactatgacatggcctgggtccgccaggctccaacgaggggtctggagtgggtcgcttccattagtcctggtggtggaaaaacttactatcgagactccgtgaagggccgactcactatctccagaaacaatgcagaaaatacccaatacctgcaaattgacagtctgaggtctgaagacacggccacttattactgttcaagactcggtccggcatactccggagaatggtttgcttactggggccaaggcactctggtcactgtctcttca

SEQ ID NO: 390 - DNA, 318 bp, synthetic construct
  misc_feature 1..318: note = Synthetic: ZNRF3-55 (VL DNA)
  source 1..318: mol_type = other DNA
gacactgtgctgacccagtctcctgctttgactgtgtctccaggagacaaaattaccatctcctgtagggccagtgaaggtgtcaatacacgtatacactggtaccaacagaaatcaggacagcaacccaaactcctcatctacggtgcatccaacttagattctggagtccctgacaggttcagtggcagtgggtttgggacagactttaccctcaccatcgatcctgttgaggctagtgatactgccacctatttctgtcagcagagttggaatgttccgcacacgtttggaggtgggaccaaactggaactgaaa

SEQ ID NO: 391 - protein, 121 aa, synthetic construct
  REGION 1..121: note = Synthetic: ZNRF3-55 (VH prot)
EVHLVESGGGLVQPGGSLKLSCAASGFTFSNYDMAWVRQAPTRGLEWVASISPGGGKTYYRDSVKGRLTISRNNAENTQYLQIDSLRSEDTATYYCSRLGPAYSGEWFAYWGQGTLVTVSS

SEQ ID NO: 392 - protein, 106 aa, synthetic construct
  REGION 1..106: note = Synthetic: ZNRF3-55(VL prot)
DTVLTQSPALTVSPGDKITISCRASEGVNTRIHWYQQKSGQQPKLLIYGASNLDSGVPDRFSGSGFGTDFTLTIDPVEASDTATYFCQQSWNVPHTFGGGTKLELK

SEQ ID NO: 393 - DNA, 351 bp, synthetic construct
  misc_feature 1..351: note = Synthetic: ZNRF3-6 (VH DNA)
  source 1..351: mol_type = other DNA
gaggtacagctggtggagtctggaggaggcttagtgccgcctggaaagtccctgaaactctcctgttcagcctctggattcccattcagtaactatggcatgcactggattcgccaagctccaggaaaggggcttgattgggttggatacattagtagtaatagtggtacaatctatgcagacgctgtgaagggccggttcaccatctccagagacaatgcaaagaacaccctgtacctgctaatcaacagtctgaagtctgaagacactgccatgtattactgtgcaagaggttattttgatggttattatcgtttctggggccaaggagtcatggtcacagtctcctca

SEQ ID NO: 394 - DNA, 336 bp, synthetic construct
  misc_feature 1..336: note = Synthetic: ZNRF3-6 (VL DNA)
  source 1..336: mol_type = other DNA
gacattgtgatgacccagtctccattctccctggctgtgtcagaaggagacatggtcactataatgtgcaggtccagtcagagtcttctatccagtggaaaccaaaagaactacttggcctggtaccagcagaaaccagggcagtctcctaaactactgatctaccatgcatccactagacaatcaggggtccctgatcgcttcataggcagtggatctgggacagacttcactctgaccatcagcgatgtgcaggctgaagacctggcagattattactgcctgcagcattacagttctcccacgttcggctcagggacgaagttggaaataaaa

SEQ ID NO: 395 - protein, 117 aa, synthetic construct
  REGION 1..117: note = Synthetic: ZNRF3-6 (VH prot)
EVQLVESGGGLVPPGKSLKLSCSASGFPFSNYGMHWIRQAPGKGLDWVGYISSNSGTIYADAVKGRFTISRDNAKNTLYLLINSLKSEDTAMYYCARGYFDGYYRFWGQGVMVTVSS

SEQ ID NO: 396 - protein, 112 aa, synthetic construct
  REGION 1..112: note = Synthetic: ZNRF3-6(VL prot)
DIVMTQSPFSLAVSEGDMVTIMCRSSQSLLSSGNQKNYLAWYQQKPGQSPKLLIYHASTRQSGVPDRFIGSGSGTDFTLTISDVQAEDLADYYCLQHYSSPTFGSGTKLEIK

SEQ ID NO: 397 - DNA, 372 bp, synthetic construct
  misc_feature 1..372: note = Synthetic: ZNRF3-90 (VH DNA)
  source 1..372: mol_type = other DNA
gaggtgcagttggtggagtctgggggaggcttagtgcagcctggaaggtccatgaagctctcctgtgcagcctcaggattcactttcagtaactattacatggcctgggtccgccaggctccaaggaagggtctggagtgggtcgcatccattactactggtggaactaacacttactatcgagactccgtgaggggccgattcactatctccagagataatgcaaaaagcaccctatacctgcaaatggacagtctgaggtctgaggaaacggccacttattactgtgcaagacattcggggggtttaattcgggcccggggccactactttcattactggggccaaggagtcatggtcacagtctcctca

SEQ ID NO: 398 - DNA, 321 bp, synthetic construct
  misc_feature 1..321: note = Synthetic: ZNRF3-90 (VL DNA)
  source 1..321: mol_type = other DNA
gacatccagatgacacagtctccagcttccctgtctgcatctcttggagaaattgtctccatcgaatgtctagcaagtgagggcatttccaatgatttagcgtggtatcagcagaagtcagggaaatctcctcaggtcctggtctatgctgcaagtaggttgcaagacggggtcccatcacggttcagtggcagtggatctggcacacggtattctctcaagatcagcggcatgcaacctgaagatgaagcagattatttctgtcaacagagttacacgtatccgtacacgtttggagctgggaccaagctggaactgaaa

SEQ ID NO: 399 - protein, 124 aa, synthetic construct
  REGION 1..124: note = Synthetic: ZNRF3-90 (VH prot)
EVQLVESGGGLVQPGRSMKLSCAASGFTFSNYYMAWVRQAPRKGLEWVASITTGGTNTYYRDSVRGRFTISRDNAKSTLYLQMDSLRSEETATYYCARHSGGLIRARGHYFHYWGQGVMVTVSS

SEQ ID NO: 400 - protein, 107 aa, synthetic construct
  REGION 1..107: note = Synthetic: ZNRF3-90(VL prot)
DIQMTQSPASLSASLGEIVSIECLASEGISNDLAWYQQKSGKSPQVLVYAASRLQDGVPSRFSGSGSGTRYSLKISGMQPEDEADYFCQQSYTYPYTFGAGTKLELK

SEQ ID NO: 401 - protein, 109 aa, synthetic construct
  REGION 1..109: note = Synthetic: RNF43-1 (VH prot)
QSVEESGGRLVTPGTPLILTCTVSGIDLSRYNMNWVRQAPGKGLEYIGYIYYGTGTTYYASWVKGRFTISKTSTTVDLKITSPTTEDTATYFCASGDFWGPGTLVTVSS

SEQ ID NO: 402 - protein, 109 aa, synthetic construct
  REGION 1..109: note = Synthetic: RNF43-1(VL prot)
DIVMTQTPASVEAAVGGTVTINCQASQSISSYLAWYQQKPGQPPKLLIYYASNLESGVPSRFKGSGSGTQFTLTISDLECADAATYYCQAYYSSITGWAFGGGTEVVVK

SEQ ID NO: 403 - protein, 113 aa, synthetic construct
  REGION 1..113: note = Synthetic: RNF43-24 (VH prot)
QEQLVESGGGLVQPGESLTLSCKASGFSFSTGYDMCWVRQAPGKGLEWIGCIGTSSGDTGYASWAKGRFTISKASSTTVTLQMTSLTAADTATYFCARGFSLWGPGTLVTVSS

SEQ ID NO: 404 - protein, 113 aa, synthetic construct
  REGION 1..113: note = Synthetic: RNF43-24(VL prot)
AQVLTQTASPVSAAVGGTVTINCQSSQSVYSNNRLGWYQQKPGQPPRLLIYQTSKLASGVPSRFRGSGSGTQFTLTISDLECDDAATYYCLGIYDCSSADCTAFGGGTEVVVK

SEQ ID NO: 405 - protein, 113 aa, synthetic construct
  REGION 1..113: note = Synthetic: RNF43-8 (VH prot)
QSVEESGGRLVTPGTPLTLTCTVSGFSLSSYAMAWVRQAPGEGLEWIGTINRAGNTYYASWAKGRFTISKTSTTVDLKVTSPTTEDTATYFCARSLYSTNNLWGPGTLVTVSS

SEQ ID NO: 406 - protein, 112 aa, synthetic construct
  REGION 1..112: note = Synthetic: RNF43-8(VL prot)
AIVMTQTPSSKSVPVGGTVTISCQASESLYDNDDLSWFQQKPGQPPKLLIYQASSLASGVPSRFKGSGSGTQFTLTISDVVCGDAATYYCAGYAGSSTDGVAFGGGTEVVVK

SEQ ID NO: 407 - protein, 119 aa, synthetic construct
  REGION 1..119: note = Synthetic: RNF43-12 (VH prot)
QEQLVESGGGLVKPGASLTLTCKASGFSLSNGDVMCWVRQAPGKGLEWIGCIGISNNDRAAYASWAKGRFTISKTSSTTVTLQTTSLTAADSATYFCARYLASEYYNLWGPGTLVTGSS

SEQ ID NO: 408 - protein, 110 aa, synthetic construct
  REGION 1..110: note = Synthetic: RNF43-12(VL prot)
AYDMTQTPASVEAAVGGTITINCQASESISSWLAWYQQKPGQPPKLLIYKASTLASGVSSRFEGSGSGTEFTLTISDLECADAATYYCQQGASWSNVENTFGGGTEVVVK

SEQ ID NO: 409 - protein, 119 aa, synthetic construct
  REGION 1..119: note = Synthetic: RNF43-20 (VH prot)
REQLVESGGGLVQPEGSLTLTCKASGFDFSSNVMCWVRQAPGKGLEWIGCIYTGSGSTAYASWAKGRFTITRSTSLNTVTLQMTSLTAADTATYFCARYVSGGEYYNLWGPGTLVTVSS

SEQ ID NO: 410 - protein, 110 aa, synthetic construct
  REGION 1..110: note = Synthetic: RNF43-20(VL prot)
ALVMTQTPASVEAAVGGTVTIKCQASQSISSYLNWYQQKPGQPPKLLIYKASTLASGVSSRFKGSGSGTQFTLTISDLECADAATYYCQQGSSWNSIENIFGGGTEVVVK

SEQ ID NO: 411 - protein, 120 aa, synthetic construct
  REGION 1..120: note = Synthetic: RNF43-11 (VH prot)
QEQLVESGGGLVQPEGSLTLTCKASGFDFSSNVMCWVRQAPGKGLEWIGCIYTGSSGSTYYASWAKGRFTITRSTSLNTVTLQMTSLTAADTATYFCARYVSGGEYYNLWGPGTLVTVSS

SEQ ID NO: 412 - protein, 110 aa, synthetic construct
  REGION 1..110: note = Synthetic: RNF43-11(VL prot)
ALVMTQTPASVEAAVGGTVTINCQASESISNWLAWYQQKPGQPPKLLIYKASTLASGVSSRFKGSGSGTQFTLTISDLECADAATYYCQQGSSWINIEDVFGGGTEVVVK

SEQ ID NO: 413 - protein, 119 aa, synthetic construct
  REGION 1..119: note = Synthetic: RNF43-23 (VH prot)
QEQVVESGGGLVQPEGSLTLTCKGSGFDFSGNVLCWVRQAPGKGLEWIGCIATGIGSTAYASWAKGRFTITRGTSLNTVDLKMTSLTAADTATYFCARYVSGSEYYNFWGPGTLVTVSS

SEQ ID NO: 414 - protein, 110 aa, synthetic construct
  REGION 1..110: note = Synthetic: RNF43-23(VL prot)
AYDMTQTPASVEVAVGGTVTIKCQASQSISSFLAWYQQKPGQPPMLLIYKASTLASGVPSRFKGSGSGTQFTLTISGVECADAATYYCQQGYSWNGVDNTFGGGTEVVVK

SEQ ID NO: 415 - protein, 115 aa, synthetic construct
  REGION 1..115: note = Synthetic: RNF43-6 (VH prot)
QSLEESGGRLVTPGTPLTLTCTVSGFSLSAFGMNWVRQAPGKGLEWIGYISAGGSAVYPNWAEGRFTISKASTTVTLKMTSLTTEDTATYFCVRGGYVGSTNSLWGPGTLVTVSS

SEQ ID NO: 416 - protein, 111 aa, synthetic construct
  REGION 1..111: note = Synthetic: RNF43-6(VL prot)
AAVLTQTPSPVSAAVGGTVSISCQSSKSVHNNNWLAWYQQKPGQPPKLLIYGASTLASGVPSRFKGSGSGTQFTLTISDVQCDDAATYYCSGGYSSIIDNGFGGGTDVVVE

SEQ ID NO: 417 - protein, 115 aa, synthetic construct
  REGION 1..115: note = Synthetic: RNF43-5 (VH prot)
QSVEESGGRLVTPGTPLTLTCTVSGFSLNTYAMGWVRQAPGKGLEWIGTIGSSGSTYYATWAKGRFTVSKTSTTVDLKITSPTTEDTATYFCVRGADGLNYYPIWGPGTLVTVSL

SEQ ID NO: 418 - protein, 113 aa, synthetic construct
  REGION 1..113: note = Synthetic: RNF43-5(VL prot)
AQVLTQTPSSVSAAVGGTVTINCQASQSVYNNKNLAWYQQKPGQPPKLLIYDASDLASGVSSRFKGSGSGTQFTLTISGVQCDDAATYYCQGEFNCDKGDCNAFGGGTEVVVK

SEQ ID NO: 419 - protein, 119 aa, synthetic construct
  REGION 1..119: note = Synthetic: RNF43-15 (VH prot)
QEQLEESGGDLVKPGASLTLTCTASRFSFSNDDVICWVRQAPGKGLEWIGCIATINGNTAYATWAKGRFTSSKTSSTTVTLQMTRLTAADTATYFCARWDTGSEHVTLWGPGTLVTVSS

SEQ ID NO: 420 - protein, 110 aa, synthetic construct
  REGION 1..110: note = Synthetic: RNF43-15(VL prot)
AYDMTQTPASVEVAVGGTVTIKCQASQSISSYLAWYQQKPGQPPKPLIYEASKLASGVPSRFSGSGSGTQFTLTISGVQCADAATYYCQQGASWSNVDNVFGGGTEVVVK

SEQ ID NO: 421 - protein, 119 aa, synthetic construct
  REGION 1..119: note = Synthetic: RNF43-2 (VH prot)
QSLEESGGDLVKPGASLTLTCTASGFSFSSSYYMCWVRQAPGKGLELIGCGFTGRHYTYYASWAKGRFTISKSSSTTVTLQMTSLTAADTATYFCARKSGSDYDYFDLWGPGTLVTVSS

SEQ ID NO: 422 - protein, 110 aa, synthetic construct
  REGION 1..110: note = Synthetic: RNF43-2(VL prot)
AYDMTQTPASVEAVVGGTVTIKCQASQSINSWLSWYQQKPGQRPKLLIYRASTLASGVSSRFKGSGSGTEFTLTISDLECADAATYYCQQGYVSSNINNVFGGGTEVVVK

SEQ ID NO: 423 - protein, 117 aa, synthetic construct
  REGION 1..117: note = Synthetic: RNF43-16 (VH prot)
QSVEESGGRLVTPGTPLTLTCTVSGFFLSSYYMSWVRQAPGKGLEWIGYIYAGSGSTYYASWAKGRFTISKTSTTVDLKITSPTTEDTATYFCVRDINGGDYGMDPWGPGTLVTVSS

SEQ ID NO: 424 - protein, 113 aa, synthetic construct
  REGION 1..113: note = Synthetic: RNF43-16(VL prot)
DIVMTQTPASVSAAVGGTVTIKCQASESIDSGLAWYQQKPGQRPKLLIYAASNLASGVSSRFKGSGSGTEYSLTISGVQCDDAATYYCQCTYGSVTSNTYGIAFGGGTEVVVK

SEQ ID NO: 425 - protein, 116 aa, synthetic construct
  REGION 1..116: note = Synthetic: RNF43-14 (VH prot)
QSVEESGGRLVTPGTPLTLTCTVSGIDLSSYAMGWVRQAPGKGLEYIGFIYASGSTYYASWAKGRFTISKTSTTVDLKITSPTTEDTATYFCARDSSETINYFNIWGPGTLVTVSL

SEQ ID NO: 426 - protein, 110 aa, synthetic construct
  REGION 1..110: note = Synthetic: RNF43-14(VL prot)
NIVMTQTPSPVSGAVGGTVTIKCQASQSISNWLAWYQQKPGQPPKVLIYRASTLASGVSSRFKGSGSGTEFTLTISDLECADAATYYCQQGYYISYLDNAFGGGTEVVVK

SEQ ID NO: 427 - protein, 116 aa, synthetic construct
  REGION 1..116: note = Synthetic: RNF43-17 (VH prot)
QSVEESGGRLVTPGTPLTLTCTGSGFSLSNYAMGWVRQAPGKGLEYIGIIYASGSTWYASWAKGRFTISKTSTTVDLKITSPATEDTATYFCCRDATTTYDRLDLWGQGTLITVSS

SEQ ID NO: 428 - protein, 110 aa, synthetic construct
  REGION 1..110: note = Synthetic: RNF43-17(VL prot)
AFELTQTPSSVEAAVGGTVTIKCQASQSISKWLAWYQQKPGQPPKLLIYRASTLPSGVPSRFKGSGSGTEFTLTISGVECADAATYYCQQGYSVTNLDEAFGGGTEVVVK

SEQ ID NO: 429 - protein, 123 aa, synthetic construct
  REGION 1..123: note = Synthetic: RNF43-22 (VH prot)
QEQLEESGGDLVKPGASLTLTCTASGFSVSNSYYMCWVRQAPGKGLEWIACINTGSSGSTYYASWAKGRFTISKTSSTTVTLQMTTLTAADTATYFCARGYAGYAGYGQFNLWGPGTLVTVSS

SEQ ID NO: 430 - protein, 110 aa, synthetic construct
  REGION 1..110: note = Synthetic: RNF43-22(VL prot)
AYDMTQTPASVEVAVGGTVTIKCQASQSISSYLAWYQQKPGQRPKVLIYDVSKLASGVPSRFKGSGSGTQFTLTISGVECADAATYYCQQGYSMSNVDNTFGGGTEVVVK

SEQ ID NO: 431 - protein, 122 aa, synthetic construct
  REGION 1..122: note = Synthetic: RNF43-9 (VH prot)
QSLEESGGDLVQPEGSLTLTCTASGFSFSSSCYMCWVRQAPGKGLEWIACINTGSSGSTYYASWAKGRFTISKTSSTTVTLQMTILTAADTATYFCARGYAGYTGYGQFNLWGPGTLVTVSS

SEQ ID NO: 432 - protein, 110 aa, synthetic construct
  REGION 1..110: note = Synthetic: RNF43-9(VL prot)
QIVMTQTPASVEAAVGGTVTIKCQASQSISNYLAWYQQKPGQRPKLLIYDASKLASGVPSRFKGSGSGTQFTLTISGVECADAATYYCQQGYTMNNVDNAFGGGTEVVVK

SEQ ID NO: 433 - protein, 122 aa, synthetic construct
  REGION 1..122: note = Synthetic: RNF43-21 (VH prot)
QSLEESGGDLVKPGASLTLTCTASGFSFSSSYYMCWVRQAPGKGLEWIACIYAGSSGSTYYASWAKGRFTVSKTSSTTVTLQMTSLTAADTATYFCARYTYGSGSDSYFNLWGPGTLVTVSS

SEQ ID NO: 434 - protein, 110 aa, synthetic construct
  REGION 1..110: note = Synthetic: RNF43-21(VL prot)
AYDMTQTPASVEAAVGGTVTIQCQASQSIGSYLAWYQQKPGQPPKLLIYRASTLSSGVPSRFKGSGSGTQFTLTISDLECADAATYYCQQGYSITNLDNGFGGGTEVVVK

SEQ ID NO: 435 - protein, 124 aa, synthetic construct
  REGION 1..124: note = Synthetic: RNF43-13 (VH prot)
QSLEESGGGLVKPGGTLTLTCKASGIDFSGYYYMCWVRQAPGKGLEWIACIYTTSGSAWYASWAKGRFTISITTSLNTVTLQMTSLTATDTATYFCVRYANYGGYNYPEFGVLWGPGTLVTVSS

SEQ ID NO: 436 - protein, 110 aa, synthetic construct
  REGION 1..110: note = Synthetic: RNF43-13(VL prot)
AYDMTQTPASVEVAVGATVTIKCQASEDIYNLLAWYQQKPGQPPKLLIYRASKLASGVPSRFSGSGSGTEFTLTISSVQCDDAATYYCQQGYSISNVENPFGGGTEVVVK

SEQ ID NO: 437 - protein, 124 aa, synthetic construct
  REGION 1..124: note = Synthetic: RNF43-19 (VH prot)
QQQLEESGGGLVKPGGTLTLTCKASGIDFSTYYYMCWVRQAPGKGLEWIACIYTGDGNTYYASWAKGRFTISKTSSTTVTLQMTSLTAADTATYFCARDLAGSSYYSGYYFKLWGPGTLVTVSS

SEQ ID NO: 438 - protein, 108 aa, synthetic construct
  REGION 1..108: note = Synthetic: RNF43-19(VL prot)
DVVMTQTPASVSEPVGGTVTIKCQASQSIGSNLAWYQQKPGQPPKLLIYSVSNLASGVSSRFKGSRSGTEYTLTISDLECADAATYYSQSGYYGSTYAFGGGTEVVVK

SEQ ID NO: 439 - protein, 116 aa, synthetic construct
  REGION 1..116: note = Synthetic: ZNRF3-331 (VH prot)
QSVEESGGRLVTPGTPLTLTCTVSGFSLSSYAMGWVRQAPGKGLEWIGIIYAGDTTYYASWAKGRFTISETSTTVDLKITSPTTEDTATYFCARKSDDDSDSYLLWGPGTLVTVSS

SEQ ID NO: 440 - protein, 110 aa, synthetic construct
  REGION 1..110: note = Synthetic: ZNRF3-331(VL prot)
AYDMTQTPASVEVAVGGTVTIKCQASQSISSYLNWYQQKRGQPPKLLIYDASTLASGVPSRFSGSGSGTEFTLTISGVQCDDAATYYCQQGWSSTNIDNIFGGGTEVVVK

SEQ ID NO: 441 - protein, 123 aa, synthetic construct
  REGION 1..123: note = Synthetic: ZNRF3-333 (VH prot)
QSLEESGGGLVQPEGSLTLTCTASGIDFSARSYMCWVRQAPGKGLEWVACIYTGSSGSTYYANWAKGRFTISKTSSTTVTLRMTSLTAADTASYFCARGAYVGYGFAAYFTLWGPGTLVTVSS

SEQ ID NO: 442 - protein, 109 aa, synthetic construct; identical to SEQ ID NO: 402
  REGION 1..109: note = Synthetic: ZNRF3-333(VL prot)

SEQ ID NO: 443 - protein, 122 aa, synthetic construct
  REGION 1..122: note = Synthetic: ZNRF3-332 (VH prot)
QSLEESGGGLVQPEGSLTLTCTASGIDFSTRNYMCWVRQAPGKGLEWVGCIYTSSGGTYYASWVNGRFTISKTSSTTVTLRMTSLTAADTATYFCARGGYVGYGYATYFNLWGPGTLVAVSS

SEQ ID NO: 444 - protein, 109 aa, synthetic construct
  REGION 1..109: note = Synthetic: ZNRF3-332(VL prot)
DVVMTQTPASVSEPVGGTVTIKCQASQSISSYLSWYQQKPGQPPKLLIYYVSNLESGVPSRFKGSGSGTEFTLTISDLECADAATYYCQNYYSSSSGWAFGGGTEVVVK

SEQ ID NO: 445 - protein, 409 aa, Homo sapiens
  source 1..409: note = Human RNF13 (O43567; UNQ16056)
MLLSIGMLMLSATQVYTILTVQLFAFLNLLPVEAGKYALADASLKMADPNRFRGKDLPVLGSDILAYNFENASQTFDDLPARFGYRLPAEGLKGFLINSKPENACEPIVPPPVKDNSSGTFIVLIRRLDCNFDIKVLNAQRAGYKAAIVHNVDSDDLISMGSNDIEVLKKIDIPSVFIGESSANSLKDEFTYEKGGHLILVPEFSLPLEYYLIPFLIIVGICLILIVIFMITKFVQDRHRARRNRLRKDQLKKLPVHKFKKGDEYDVCAICLDEYEDGDKLRILPCSHAYHCKCVDPWLTKTKKTCPVCKQKVVPSQGDSDSDTDSSQEENEVTEHTPLLRPLASVSAQSFGALSESRSHQNMTESSDYEEDDNEDTDSSDAENEINEHDVVVQLQPNGERDYNIANTV

SEQ ID NO: 446 - protein, 811 aa, Homo sapiens
  source 1..811: note = Human RNF43 (Q68DV7; UNQ9671)
MSGGHQLQLAALWPWLLMATLQAGKYALADASLKMADPNRFRGKDLPVLGSGFGRTGLVLAAAVESERSAEQKAIIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDYDVWILMTVVGTIFVIILASVLRIRCRPRHSRPDPLQQRTAWAISQLATRRYQASCRQARGEWPDSGSSCSSAPVCAICLEEFSEGQELRVISCLHEFHRNCVDPWLHQHRTCPLCMFNITEGDSFSQSLGPSRSYQEPGRRLHLIRQHPGHAHYHLPAAYLLGPSRSAVARPPRPGPFLPSQEPGMGPRHHRFPRAAHPRAPGEQQRLAGAQHPYAQGWGLSHLQSTSQHPAACPVPLRRARPPDSSGSGESYCTERSGYLADGPASDSSSGPCHGSSSDSVVNCTDISLQGVHGSSSTFCSSLSSDFDPLVYCSPKGDPQRVDMQPSVTSRPRSLDSVVPTGETQVSSHVHYHRHRHHHYKKRFQWHGRKPGPETGVPQSRPPIPRTQPQPEPPSPDQQVTRSNSAAPSGRLSNPQCPRALPEPAPGPVDASSICPSTSSLFNLQKSSLSARHPQRKRRGGPSEPTPGSRPQDATVHPACQIFPHYTPSVAYPWSPEAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 447 - protein, 645 aa, Homo sapiens
  source 1..645: note = Human SYVN1 (Q86TM6; UNQ22255)
MFRTAVMMAASLALTGAVVAHAGKYALADASLKMADPNRFRGKDLPVLGSYYLKHQFYPTVVYLTKSSPSMAVLYIQAFVLVFLLGKVMGKVFFGQLRAAEMEHLLERSWYAVTETCLAFTVFRDDFSPRFVALFTLLLFLKCFHWLAEDRVDFMERSPNISWLFHCRIVSLMFLLGILDFLFVSHAYHSILTRGASVQLVFGFEYAILMTMVLTIFIKYVLHSVDLQSENPWDNKAVYMLYTELFTGFIKVLLYMAFMTIMIKVHTFPLFAIRPMYLAMRQFKKAVTDAIMSRRAIRNMNTLYPDATPEELQAMDNVCIICREEMVTGAKRLPCNHIFHTSCLRSWFQRQQTCPTCRMDVLRASLPAQSPPPPEPADQGPPPAPHPPPLLPQPPNFPQGLLPPFPPGMFPLWPPMGPFPPVPPPPSSGEAVAPPSTSAAALSRPSGAATTTAAGTSATAASATASGPGSGSAPEAGPAPGFPFPPPWMGMPLPPPFAFPPMPVPPAGFAGLTPEELRALEGHERQHLEARLQSLRNIHTLLDAAMLQINQYLTVLASLGPPRPATSVNSTEETATTVVAAASSTSIPSSEATTPTPGASPPAPEMERPPAPESVGTEEMPEDGEPDAAELRRRRLQKLESPVAH

SEQ ID NO: 448 - protein, 447 aa, Homo sapiens
  source 1..447: note = Human RNF130 (Q86XS8; UNQ4694)
MSCAGRAGPARLAALALLTCSLWPARAGKYALADASLKMADPNRFRGKDLPVLGSDNASQEYYTALINVTVQEPGRGAPLTFRIDRGRYGLDSPKAEVRGQVLAPLPLHGVADHLGCDPQTRFFVPPNIKQWIALLQRGNCTFKEKISRAAFHNAVAVVIYNNKSKEEPVTMTHPGTGDIIAVMITELRGKDILSYLEKNISVQMTIAVGTRMPPKNFSRGSLVFVSISFIVLMIISSAWLIFYFIQKIRYTNARDRNQRRLGDAAKKAISKLTTRTVKKGDKETDPDFDHCAVCIESYKQNDVVRILPCKHVFHKSCVDPWLSEHCTCPMCKLNILKALGIVPNLPCTDNVAFDMERLTRTQAVNRRSALGDLAGDNSLGLEPLRTSGISPLPQDGELTPRTGEINIAVTKEWFIIASFGLLSALTLCYMIIRATASLNANEVEWF

SEQ ID NO: 449 - protein, 333 aa, Homo sapiens
  source 1..333: note = Human RNF148 (Q8N7C7; UNQ34666)
MSFLRITPSTHSSVSSGLLRLSIFLLLSFPDSNGGKYALADASLKMADPNRFRGKDLPVLGSKAIWTAHLNITFQVGNEITSELGESGVFGNHSPLERVSGVVALPEGWNQNACHPLTNFSRPKQADSWLALIERGGCTFTHKINVAAEKGANGVIIYNYQGTGSKVFPMSHQGTENIVAVMISNLKGMEILHSIQKGVYVTVIIEVGRMHMQWVSHYIMYLFTFLAATIAYFYLDCVWRLTPRVPNSFTRRRSQIKTDVKKAIDQLQLRVLKEGDEELDLNEDNCVVCFDTYKPQDVVRILTCKHFFHKACIDPWLLAHRTCPMCKCDILKT

SEQ ID NO: 450 - protein, 428 aa, Homo sapiens
  source 1..428: note = Human RNF149 (Q8NC42; UNQ4467)
MAWRRREASVGARGVLALALLALALCVPGARGGKYALADASLKMADPNRFRGKDLPVLGSRALEWFSAVVNIEYVDPQTNLTVWSVSESGRFGDSSPKEGAHGLVGVPWAPGGDLEGCAPDTRFFVPEPGGRGAAPWVALVARGGCTFKDKVLVAARRNASAVVLYNEERYGNITLPMSHAGTGNIVVIMISYPKGREILELVQKGIPVTMTIGVGTRHVQEFISGQSVVFVAIAFITMMIISLAWLIFYYIQRFLYTGSQIGSQSHRKETKKVIGQLLLHTVKHGEKGIDVDAENCAVCIENFKVKDIIRILPCKHIFHRICIDPWLLDHRTCPMCKLDVIKALGYWGEPGDVQEMPAPESPPGRDPAANLSLALPDDDGSDDSSPPSASPAESEPQCDPSFKGDAGENTALLEAGRSDSRHGGPIS

SEQ ID NO: 451 - protein, 660 aa, Homo sapiens
  source 1..660: note = Human LNX1-isoform2 (Q8TBB; UNQ 574)
MKALLLLVLPWLSPAGKYALADASLKMADPNRFRGKDLPVLGSNYIDNVGNLHFLYSELCKGASHYGLTKDRKRRSQDGCPDGCASLTATAPSPEVSAAATISLMTDEPGLDNPAYVSSAEDGQPAISPVDSGRSNRTRARPFERSTIRSRSFKKINRALSVLRRTKSGSAVANHADQGRENSENTTAPEVFPRLYHLIPDGEITSIKINRVDPSESLSIRLVGGSETPLVHIIIQHIYRDGVIARDGRLLPGDIILKVNGMDISNVPHNYAVRLLRQPCQVLWLTVMREQKFRSRNNGQAPDAYRPRDDSFHVILNKSSPEEQLGIKLVRKVDEPGVFIFNVLDGGVAYRHGQLEENDRVLAINGHDLRYGSPESAAHLIQASERRVHLVVSRQVRQRSPDIFQEAGWNSNGSWSPGPGERSNTPKPLHPTITCHEKVVNIQKDPGESLGMTVAGGASHREWDLPIYVISVEPGGVISRDGRIKTGDILLNVDGVELTEVSRSEAVALLKRTSSSIVLKALEVKEYEPQEDCSSPAALDSNHNMAPPSDWSPSWVMWLELPRCLYNCKDIVLRRNTAGSLGFCIVGGYEEYNGNKPFFIKSIVEGTPAYNDGRIRCGDILLAVNGRSTSGMIHACLARLLKELKGRITLTIVSWPGTFL

SEQ ID NO: 452 - protein, 456 aa, Homo sapiens
  source 1..456: note = Human RNF128 (Q8TEB7; UNQ8231)
MGPPPGAGVSCRGGCGFSRLLAWCFLLALSPQAPGSRGGKYALADASLKMADPNRFRGKDLPVLGSAEAVWTAYLNVSWRVPHTGVNRTVWELSEEGVYGQDSPLEPVAGVLVPPDGPGALNACNPHTNFTVPTVWGSTVQVSWLALIQRGGGCTFADKIHLAYERGASGAVIFNFPGTRNEVIPMSHPGAVDIVAIMIGNLKGTKILQSIQRGIQVTMVIEVGKKHGPWVNHYSIFFVSVSFFIITAATVGYFIFYSARRLRNARAQSRKQRQLKADAKKAIGRLQLRTLKQGDKEIGPDGDSCAVCIELYKPNDLVRILTCNHIFHKTCVDPWLLEHRTCPMCKCDILKALGIEVDVEDGSVSLQVPVSNEISNSASSHEEDNRSETASSGYASVQGTDEPPLEEHVQSTNESLQLVNHEANSVAVDVIPHVDNPTFEEDETPNQETAVREIKS

SEQ ID NO: 453 - protein, 404 aa, Homo sapiens
  source 1..404: note = Human (RNF133 (Q8WVZ7; UNQ34667)
MHLLKVGTWRNNTASSWLMKFSVLWLVSQNCCRAGKYALADASLKMADPNRFRGKDLPVLGSSVVWMAYMNISFHVGNHVLSELGETGVFGRSSTLKRVAGVIVPPEGKIQNACNPNTIFSRSKYSETWLALIERGGCTFTQKIKVATEKGASGVIIYNVPGTGNQVFPMFHQAFEDVVVVMIGNLKGTEIFHLIKKGVLITAVVEVGRKHIIWMNHYLVSFVIVTTATLAYFIFYHIHRLCLARIQNRRWQRLTTDLQNTFGQLQLRVVKEGDEEINPNGDSCVICFERYKPNDIVRILTCKHFFHKNCIDPWILPHGTCPICKCDILKVLGIQVVVENGTEPLQVLMSNELPETLSPSEEETNNEVSPAGTSDKVIHVEENPTSQNNDIQPHSVVEDVHPSP

SEQ ID NO: 454 - protein, 457 aa, Homo sapiens
  source 1..457: note = Human ZNRF4 (Q8WWF5; UNQ12510)
MPLCRPEHLMPRASRVPVAASLPLSHAGKYALADASLKMADPNRFRGKDLPVLGSVIPTQLPSRPGHRPPGRPRRCPKASCLPPPVGPSSTQTAKRVTMGWPRPGRALVAVKALLVLSLLQVPAQAVVRAVLEDNSSSVDFADLPALFGVPLAPEGIRGYLMEVKPANACHPIEAPRLGNRSLGAIVLIRRYDCTFDLKVLNAQRAGFEAAIVHNVHSDDLVSMTHVYEDLRGQIAIPSVFVSEAASQDLRVILGCNKSAHALLLPDDPPCHDLGCHPVLTVSWVLGCTLALVVSAFFVLNHLWLWAQACCSHRRPVKTSTCQKAQVRTFTWHNDLCAICLDEYEEGDQLKILPCSHTYHCKCIDPWFSQAPRRSCPVCKQSVAATEDSFDSTTYSFRDEDPSLPGHRPPIWAIQVQLRSRRLELLGRASPHCHCSTTSLEAEYTTVSSAPPEAPGQ

SEQ ID NO: 455 - protein, 604 aa, Homo sapiens
  source 1..604: note = Human RSPRY1 (Q96DX4; UNQ328)
MIVFGWAVFLASRSLGGKYALADASLKMADPNRFRGKDLPVLGSQGLLLTLEEHIAHFLGTGGAATTMGNSCICRDDSGTDDSVDTQQQQAENSAVPTADTRSQPRDPVRPPRRGRGPHEPRRKKQNVDGLVLDTLAVIRTLVDNDQEPPYSMITLHEMAETDEGWLDVVQSLIRVIPLEDPLGPAVITLLLDECPLPTKDALQKLTEILNLNGEVACQDSSHPAKHRNTSAVLGCLAEKLAGPASIGLLSPGILEYLLQCLKLQSHPTVMLFALIALEKFAQTSENKLTISESSISDRLVTLESWANDPDYLKRQVGFCAQWSLDNLFLKEGRQLTYEKVNLSSIRAMLNSNDVSEYLKISPHGLEARCDASSFESVRCTFCVDAGVWYYEVTVVTSGVMQIGWATRDSKFLNHEGYGIGDDEYSCAYDGCRQLIWYNARSKPHIHPCWKEGDTVGFLLDLNEKQMIFFLNGNQLPPEKQVFSSTVSGFFAAASFMSYQQCEFNFGAKPFKYPPSMKFSTFNDYAFLTAEEKIILPRHRRLALLKQVSIRENCCSLCCDEVADTQLKPCGHSDLCMDCALQLETCPLCRKEIVSRIRQISHIS

SEQ ID NO: 456 - protein, 357 aa, Homo sapiens
  source 1..357: note = Human TRIM7 isoform 3 (Q9C029-4; UNQ 20983)
MTQATGQMLCLGKYALADASLKMADPNRFRGKDLPVLGSHVQVPLQLLLLGQKQMAAEQEKVGAEFQALRAFLVEQEGRLLGRLEELSREVAQKQNENLAQLGVEITQLSKLSSQIQETAQKPDLDFLQEFKSTLSRCSNVPGPKPTTVSSEMKNKVWNVSLKTFVLKGMLKKFKEDLRGELEKEEKVELTLDPDTANPRLILSLDLKGVRLGERAQDLPNHPCRFDTNTRVLASCGFSSGRHHWEVEVGSKDGWAFGVARESVRRKGLTPFTPEEGVWALQLNGGQYWAVTSPERSPLSCGHLSRVRVALDLEVGAVSFYAVEDMRHLYTFRVNFQERVFPLFSVCSTGTYLRIWP

SEQ ID NO: 457 - protein, 378 aa, Homo sapiens
  source 1..378: note = Human RNF167 (Q9H6Y7; UNQ16055)
MHPAAFPLPVVVAAVLWGAAPTRGGKYALADASLKMADPNRFRGKDLPVLGSLIRATSDHNASMDFADLPALFGATLSQEGLQGFLVEAHPDNACSPIAPPPPAPVNGSVFIALLRRFDCNFDLKVLNAQKAGYGAAVVHNVNSNELLNMVWNSEEIQQQIWIPSVFIGERSSEYLRALFVYEKGARVLLVPDNTFPLGYYLIPFTGIVGLLVLAMGAVMIARCIQHRKRLQRNRLTKEQLKQIPTHDYQKGDQYDVCAICLDEYEDGDKLRVLPCAHAYHSRCVDPWLTQTRKTCPICKQPVHRGPGDEDQEEETQGQEEGDEGEPRDHPASERTPLLGSSPTLPTSFGSLAPAPLVFPGPSTDPPLSPPSSPVILV

SEQ ID NO: 458 - protein, 466 aa, Homo sapiens
  source 1..466: note = Human RNF150 (Q9ULK6; UNQ11523)
MAMSLIQACCSLALSTWLLSFCFVHLLCLDFTVAGKYALADASLKMADPNRFRGKDLPVLGSEKEEWYTAFVNITYAEPAPDPGAGAAGGGGAELHTEKTECGRYGEHSPKQDARGEVVMASSAHDRLACDPNTKFAAPTRGKNWIALIPKGNCTYRDKIRNAFLQNASAVVIFNVGSNTNETITMPHAGVEDIVAIMIPEPKGKEIVSLLERNITVTMYITIGTRNLQKYVSRTSVVFVSISFIVLMIISLAWLVFYYIQRFRYANARDRNQRRLGDAAKKAISKLQIRTIKKGDKETESDFDNCAVCIEGYKPNDVVRILPCRHLFHKSCVDPWLLDHRTCPMCKMNILKALGIPPNADCMDDLPTDFEGSLGGPPTNQITGASDTTVNESSVTLDPAVRTVGALQVVQDTDPIPQEGDVIFTTNSEQEPAVSSDSDISLIMAMEVGLSDVELSTDQDCEEVKS

SEQ ID NO: 459 - protein, 964 aa, Homo sapiens
  source 1..964: note = Human ZNRF3 (Q9ULT6; UNQ28198)
MRPRSGGRPGATGRRRRRLRRRPRGLRCSRLPPPPPLPLLLGLLLAAAGPGAARAGKYALADASLKMADPNRFRGKDLPVLGSKETAFVEVVLFESSPSGDYTTYTTGLTGRFSRAGATLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHRPPRQPTEYFDMGIFLAFFVVVSLVCLILLVKIKLKQRRSQNSMNRLAVQALEKMETRKFNSKSKGRREGSCGALDTLSSSSTSDCAICLEKYIDGEELRVIPCTHRFHRKCVDPWLLQHHTCPHCRHNIIEQKGNPSAVCVETSNLSRGRQQRVTLPVHYPGRVHRTNAIPAYPTRTSMDSHGNPVTLLTMDRHGEQSLYSPQTPAYIRSYPPLHLDHSLAAHRCGLEHRAYSPAHPFRRPKLSGRSFSKAACFSQYETMYQHYYFQGLSYPEQEGQSPPSLAPRGPARAFPPSGSGSLLFPTVVHVAPPSHLESGSTSSFSCYHGHRSVCSGYLADCPGSDSSSSSSSGQCHCSSSDSVVDCTEVSNQGVYGSCSTFRSSLSSDYDPFIYRSRSPCRASEAGGSGSSGRGPALCFEGSPPPEELPAVHSHGAGRGEPWPGPASPSGDQVSTCSLEMNYSSNSSLEHRGPNSSTSEVGLEASPGAAPDLRRTWKGGHELPSCACCCEPQPSPAGPSAGAAGSSTLFLGPHLYEGSGPAGGEPQSGSSQGLYGLHPDHLPRTDGVKYEGLPCCFYEEKQVARGGGGGSGCYTEDYSVSVQYTLTEEPPPGCYPGARDLSQRIPIIPEDVDCDLGLPSDCQGTHSLGSWGGTRGPDTPRPHRGLGATREEERALCCQARALLRPGCPPEEAGAVRANFPSALQDTQESSTTATEAAGPRSHSADSSSPGA

(19) United States
(12) Patent Application Publication
(10) Pub. No.: US 2024/0101708 A1
(43) Pub. Date: Mar. 28, 2024

(54) Multispecific Binding Protein Degrader Platform and Methods of Use

(71) Applicant: Genentech Inc., South San Francisco, CA (US)

(72) Inventors: Nicholas John Agard, South San Francisco, CA (US); Felipe de Sousa e Melo, South San Francisco, CA (US); Jing Li, South San Francisco, CA (US); Hadir Mohamed Gehad Mohamed Fouad Elsayed Marei, South San Francisco, CA (US); Wen-Ting K. Tsai, South San Francisco, CA (US); Frederic Joseph De Sauvage, South San Francisco, CA (US); Jieyan He, South San Francisco, CA (US); Yee Seir Kee, South San Francisco, CA (US)

(73) Assignee: Genentech Inc., South San Francisco, CA (US)

(21) Appl. No.: 18/363,155

(22) Filed: Aug. 1, 2023

Related U.S. Application Data
(63) Continuation of application No. PCT/US2022/014938, filed on Feb. 2, 2022.
(60) Provisional application No. 63/274,288, filed on Nov. 1, 2021.
(60) Provisional application No. 63/217,470, filed on Jul. 1, 2021.
(60) Provisional application No. 63/145,336, filed on Feb. 3, 2021.

(51) Int. Cl. C07K 16/40; A61P 35/00

(52) U.S. Cl. CPC C07K 16/40; A61P 35/00; C07K 2317/14; C07K 2317/31; C07K 2317/565

(57) ABSTRACT

The present disclosure relates to a multispecific antibody platform for targeted degradation of cell surface proteins. The disclosure relates to multispecific (e.g., bispecific or trispecific) binding molecules such as multispecific antibodies that target at least one transmembrane E3 ubiquitin ligase protein and at least one cell surface protein, for example, a cell surface protein that is intended for degradation, and methods of using the same.

### CROSS-REFERENCE TO RELATED APPLICATIONS

This application is a continuation of International Patent Application No. PCT/US2022/014938, filed Feb. 2, 2022, which claims priority to U.S. Provisional Application No. 63/145,336, filed Feb. 3, 2021, and U.S. Provisional Application No. 63/217,470, filed Jul. 1, 2021, and U.S. Provisional Application No. 63/274,288, filed Nov. 1, 2021, the disclosures of each of which are hereby incorporated by reference in their entireties.

### SEQUENCE LISTING

The present application contains a Sequence Listing which has been submitted electronically in XML format. Said XML copy, created on Jul. 19, 2023 is named “2023-07-19_01164-0011-00US-ST26” and is 696,264 bytes in size. The information in the electronic format of the sequence listing is incorporated herein by reference in its entirety.

### FIELD

The present disclosure relates to a multispecific antibody platform for targeted degradation of cell surface proteins. The disclosure relates to multispecific (e.g., bispecific or trispecific) binding molecules such as multispecific antibodies that target at least one transmembrane E3 ubiquitin ligase protein and at least one cell surface protein, for example, a cell surface protein that is intended for degradation, and methods of using the same.

### BACKGROUND

Various platforms have been constructed for the purpose of targeting the degradation of a protein of interest. For example, PROTACs (proteolysis targeting chimeras) are small-molecule constructs intended to target cytosolic proteins to the 26S proteosome for degradation. They may include a domain binding to a ubiquitin E3 ligase, a domain binding to a protein targeted for degradation (i.e., a protein of interest) and a linker molecule to link the two binding domains. PROTACs, however, target cytosolic protein domains, meaning that this approach cannot be used with certain membrane-bound or cell surface protein targets. (See, e.g., Ahn et al., ChemRxiv, doi.org/10.26434/chemrxiv.12736778.v1, posted Jul. 30, 2020.) A LYTAC (lysosome targeting chimera) construct, for example, is a multivalent construct that binds to a membrane protein of interest and that also includes a ligand such as mannose-6-phosphonate that binds to the cation-independent mannose-6-phosphate receptor (CI-M6PR). Linking the targeted membrane protein to CI-M6PR brings the protein to the lysosome for degradation. (See Id.) However, unlike a PROTAC, a LYTAC requires a chemical conjugation, for example, to a mannose-6-phosphonate, which may need to be controlled during manufacturing, such as to avoid product heterogeneity. In addition, since CI-M6PR is expressed in nearly all tissues, this technique may not be useful for selectively degrading a protein in a specific tissue or tissues. A bispecific antibody that binds a cell surface E3 ubiquitin ligase, RNF43, and the cell-surface immune checkpoint protein, PD-L1, was described by Cotton et al. (J. Am. Chem. Soc. 2021, available at https://dx.doi.org/10.1021/jacs.0O10008). Lysosomal degradation of PD-L1 in a tumor cell line exposed to the bispecific antibody in vitro was demonstrated. WO2021/176034 describes bispecific single chain (VHH) “anti-tag” antibodies that were capable of degrading transiently expressed tagged cell surface proteins in HEK293T cells that also transiently expressed tagged E3 ligases. The general applicability of the approach including efficiency of degrading endogenous cell surface proteins in cells expressing an endogenous E3 ligase and effectiveness in vivo, however, remains unknown as well as optimal attributes of a platform that utilizes antibodies against cell surface ligases.

### SUMMARY

The present disclosure relates to an improved approach to target degradation of membrane-bound and cell surface proteins, which uses multispecific binding proteins, such as multispecific (e.g., bispecific or trispecific) antibodies that bind to at least one transmembrane E3 ubiquitin ligase, such as RNF43 or ZNRF3, or such as RNF128, RNF130, RNF133, RNF148, RNF149, RNF150, RNF167, or ZNRF4, or other cell surface ligases, as well as to a cell surface protein intended for degradation. The multispecific binding proteins use the transmembrane E3 ubiquitin ligase, for example, to target the cell surface protein of interest to lysosomes for degradation. Zinc and RING finger 3 (ZNRF3) and its homolog, RING finger 43 (RNF43), are exemplary transmembrane E3 ubiquitin ligases that normally promote the degradation and turnover of the Frizzled (FZD) and LRP6 receptors on the cell surface (Hao et al., Nature 485:195-200 (2012); Koo et al., Nature 488:665-669 (2012)). RNF43 and ZNRF3 are also members of the Wnt signaling pathway, which may be activated, for example, in certain types of cancers. (See, e.g., FIG. 1C.) Thus, tumor cells from various cancers may express relatively high levels of RNF43 and ZNRF3 in comparison to other cells, which, in some embodiments, may allow for selective degradation of target proteins in tumor cells. A variety of other transmembrane E3 ubiquitin ligases may also be used as targets for multispecific binding proteins herein. Optimal attributes of the multispecific antibodies are also provided herein, including for example, optimized binding affinities and multispecific formats. In some embodiments, multispecific binding proteins herein are called PROTABs (“proteolysis targeting antibodies”).

In addition to providing a general platform of multispecific binding proteins targeting a transmembrane E3 ubiquitin ligase and a cell surface protein for destruction, the present disclosure also provides a variety of antibody heavy and light chain variable regions targeting RNF43 or ZNRF3 that may be useful in constructing such multispecific antibodies and binding proteins. Binding proteins herein may, in some embodiments, be used therapeutically, for example, to degrade proteins that contribute to disease progression, for example but not limited to, in cells in which the Wnt pathway is activated and that express transmembrane E3 ubiquitin ligases. For example, certain cancers such as colorectal cancer (CRC) are characterized by overexpression of RNF43 and ZNRF3. Thus, multispecific binding proteins herein that target one or both of those ligases may be selectively targeted to cancer cells due to the cells' overexpression of these proteins. Binding proteins herein may also be used in vitro, for example, to degrade a particular membrane protein in cell culture or tissue assays and thereby reduce its level and knock down its associated signaling. For example, certain E3 ubiquitin ligases such as RNF130, RNF149, and RNF167 are expressed in hematopoietic cells, and multispecific binding proteins that target those ligases may be used, in some embodiments, to target hematopoietic cells in vivo or in vitro. Similarly, E3 ubiquitin ligases such as RNF133 and RNF148 are expressed in testicular cells, and multispecific binding proteins that target those ligases may be used, in some embodiments, to target testicular cells in vivo or in vitro.

The invention provides, inter alia, multispecific binding proteins that bind to at least a first cell surface target protein and a second cell surface protein, wherein the first cell surface target protein is a transmembrane E3 ubiquitin ligase. In some aspects, a multispecific binding protein reduces the level of the second cell surface protein on the surface of a cell compared to the level observed in the absence of the multispecific binding protein. In certain aspects, a multispecific binding protein reduces the level of the second cell surface protein on the surface of a cell in vitro compared to the level observed in the absence of the multispecific binding protein. In some such cases, the multispecific binding protein reduces the level of the second cell surface protein on the surface of a cell in vitro as determined by flow cytometry or by luminescense assay. In some cases, a multispecific binding protein reduces the level of the second cell surface protein on the surface of a cell in vivo compared to the level observed in the absence of the multispecific binding protein, or both in vitro and in vivo.

In some aspects, a multispecific binding protein herein is a multispecific antibody. For example, in some aspects, the protein is a bispecific or trispecific antibody, such as a 1+1 FabIgG, a 1+1 FvIgG, a 2+1 FvIgG, 2+1 FabIgG, a one-armed FvIgG, or a one-armed FabIgG.

In some such cases, protein comprises IgG Fc regions comprising at least one knob-into-hole modification.

In some aspects, the transmembrane E3 ubiquitin ligase does not have catalytic activity, wherein lack of catalytic activity is determined in a cell surface degradation assay. In other aspects, the transmembrane E3 ubiquitin ligase has catalytic activity, wherein presence of catalytic activity is determined in a cell surface degradation assay.

In some aspects, a multispecific binding protein herein binds to a transmembrane E3 ubiquitin ligase selected from: RNF43, ZNRF3, RNF13, RNF128, RNF130, RNF133, RNF148, RNF149, RNF150, RNF167, ZNRF4, RSPRY1, SYVN1, LNX1 isoform 2, and TRIM7 isoform 3. In some aspects, a multispecific binding protein herein binds to a transmembrane E3 ubiquitin ligase selected from: RNF43, RNF128, RNF130, RNF133 RNF149, RNF150, or ZNRF3. In some aspects, the transmembrane E3 ubiquitin ligase is RNF130, RNF133, RNF149, or RNF150. In some aspects, the transmembrane E3 ubiquitin ligase is RNF130, RNF149, or RNF167. In some aspects, the transmembrane E3 ubiquitin ligase is RNF133 or RNF148. In some aspects, the transmembrane E3 ubiquitin ligase is RNF43 or ZNFR3 or both, such that the protein binds to RNF43, ZNRF3, or both RNF43 and ZNRF3, optionally wherein the protein does not block binding between RNF43 and/or ZNRF3 and FZD and/or LRP6.

In some aspects, the multispecific binding protein is a multispecific antibody that binds to RNF43 or ZNRF3; or comprises an antibody heavy chain variable region (VH) comprising (a) CDR-H1 (b) CDR-H2, and (c) CDR-H3, and a light chain variable domain (VL) comprising (d) CDR-L1, (e) CDR-L2, and (f) CDR-L3 that bind to RNF43 or ZNRF3; or comprises a VH and a VL that bind to RNF43 or ZNRF3.

In some aspects, the multispecific binding protein comprises a heavy chain variable domain (VH) comprising a heavy chain complementarity determining region 1 (CDR-H1), CDR-H2, and/or CDR-H3 of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332.

In some aspects, the multispecific binding protein comprises a heavy chain variable region (VH) comprising the CDR-H1, CDR-H2, and CDR-H3 any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332.

In some aspects, the multispecific binding protein comprises a light chain variable domain (VL) comprising a light chain complementarity determining region 1 (CDR-L1), CDR-L2, and/or CDR-L3 of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332.

In some aspects, the multispecific binding protein comprises a light chain variable region (VL) comprising the CDR-L1, CDR-L2, and CDR-L3 any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332.

In some aspects, the multispecific binding protein comprises a heavy chain variable domain (VH) comprising (a) a heavy chain complementarity determining region 1 (CDR-H1), CDR-H2, and CDR-H3 and (b) a light chain variable domain (VL) comprising (a) a light chain complementarity determining region 1 (CDR-L1), CDR-L2, and CDR-L3 of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332.

In any of the above cases, the heavy chain CDRs and/or the light chain CDRs may be Kabat, Chothia, or McCallum CDRs. The associated VH and VL amino acid and DNA sequences for the listed antibodies above are provided in the sequence table below, with SEQ ID Nos: 33-444.

In some aspects, the multispecific binding protein comprises a heavy chain variable region (VH) comprising an amino acid sequence at least 85%, at least 90%, at least 95%, at least 97%, at least 98%, or at least 99% identical to the VH of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332, optionally wherein the amino acid sequences of the CDR-H1, CDR-H2, and/or CDR-H3 of the VH are 100% identical to those of the chosen antibody.

In some aspects, the multispecific binding protein comprises a light chain variable region (VL) comprising an amino acid sequence at least 85%, at least 90%, at least 95%, at least 97%, at least 98%, or at least 99% identical to the VL of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332, optionally wherein the amino acid sequences of the CDR-H1, CDR-H2, and/or CDR-H3 of the VH are 100% identical to those of the chosen antibody.

In some aspects, the multispecific binding protein comprises a heavy chain variable region (VH) comprising an amino acid sequence of the VH of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332.

In some aspects, the multispecific binding protein comprises a light chain variable region (VL) comprising an amino acid sequence of the VL of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332. As noted above, the VH and VL amino acid and DNA sequences of the above-listed antibodies are in SEQ ID Nos: 33-444 in the sequence table below.

In some aspects, the multispecific binding protein binds to ZNRF3 and/or RNF43, and has a binding affinity for ZNRF3 or RNF43 of less than 50 nM, or less than 10 nM, or less than 1 nM, or less than 0.5 nM, or less than 0.05 nM, or between 50 nM and 10 nM, or between 10 nM and 1 nM, or between 1 nM and 0.5 nM, or between 0.5 nM and 0.05 nM, or between 0.05 nM and 0.01 nM. In some aspects, the protein is a multispecific antibody comprising a heavy chain variable region and/or a light chain variable region of a rat anti-human RNF43 B cell antibody, a rat anti-human ZNRF3 B cell antibody, a rabbit anti-human RNF43 B cell antibody, or a rabbit anti-human ZNRF3 B cell antibody. In some aspects, the protein is a trispecific antibody comprising a 2+1 FvIgG or a 2+1 FabIgG format, wherein the protein binds to both ZNRF3 and RNF43 and to at least one second cell surface protein, optionally wherein the protein does not block binding of ZNRF3 or RNF43 to FZD and LRP6.

In some aspects, a multispecific binding protein herein may be a multispecific antibody comprising a heavy chain variable region or light chain variable region that is chimeric, or comprising humanized variable regions. In some cases, the protein comprises a wild-type human Fc region. In some cases, the Fc region is an IgG1, IgG2, IgG3, or IgG4 Fc region, optionally either a wild-type human Fc region, or a human Fc region with one or more engineered mutations. In some cases, the protein comprises an Fc region having effector function. In some cases, the protein comprises a human IgG1 Fc region comprising a LALAPG mutation or a substitution at position N297, such as N297G or N297Q, and/or wherein the Fc region lacks effector function.

In some aspects, the second cell surface protein to which the multispecific binding protein binds is a receptor tyrosine kinase, a growth factor receptor, a cytokine, a mucin, a Siglec receptor, or an immune checkpoint modulator, or is HER2, HER3, IGF1R, an EGFR, an FGFR, a VEGFR, a PDGFR, EpCAM, FZD, PD-L1, CTLA4, PD-1, TIM3, LAG3, TIGIT, CEACAM1, CD25, ILT-2, ILT-3, ILT-4, ILT-5, LAIR-1, PECAM-1 (CD31), PILR-alpha, SIRL-1, or SIRP-alpha. In some cases, the second cell surface protein is HER2, EGFR, or IGF1R. In some cases, the multispecific binding protein comprises a heavy chain variable region and a light chain variable region of an anti-HER2 antibody such as 4D5, 7C2, or 2C4 or an anti-IGF1R antibody such as cixutumumab, ganitumab, dalotuzumab, figitumumab, robatumumab, teprotumumab, or istiratumab.

The present disclosure also relates to isolated nucleic acids or sets of nucleic acids (e.g., comprising two or more nucleic acids each encoding a portion of a multimeric protein such as a light chain or a heavy chain or a half antibody), encoding a multispecific binding protein as described herein. The present disclosure also relates to host cells comprising such nucleic acids or sets of nucleic acids. The present disclosure also encompasses methods of producing the multispecific binding proteins herein, comprising culturing a host cell comprising nucleic acids or sets of nucleic acids encoding the protein under conditions suitable for the expression of the protein. Such methods may further comprise recovering the protein from the host cell. The present disclosure further encompasses multispecific binding proteins produced by the methods herein.

A pharmaceutical composition may also be prepared, comprising a multispecific binding protein as described herein and a pharmaceutically acceptable carrier.

The present disclosure also encompasses multispecific binding proteins and related pharmaceutical compositions for use as medicaments, for example, for use in treating cancer, an autoimmune condition, an inflammatory condition, a neurodegenerative condition, or an infectious disease in a subject, and/or for use in reducing the level of a cell surface protein in a subject in need thereof, optionally wherein the subject has cancer, an autoimmune condition, an inflammatory condition, a neurodegenerative condition, or an infectious disease, and/or for use in increasing an immune response in a subject, such as a subject with cancer, an autoimmune condition, an inflammatory condition, a neurodegenerative condition, or an infectious disease. In some aspects, (a) the subject has a mutation in RNF43 and the multispecific binding protein does not bind to or does not activate RNF43, or (b) the subject has a mutation in ZNRF3 and the multispecific binding protein does not bind to or does not activate ZNRF3. In some such cases, where the subject has an RNF43 or ZNRF3 mutation, the subject has a cancer in which the cancer comprises the mutation.

The present disclosure also relates to use of a multispecific binding protein or pharmaceutical composition herein in the manufacture of a medicament for treatment of cancer, an autoimmune condition, an inflammatory condition, a neurodegenerative condition, or an infectious disease in a subject; and/or for reducing the level of a cell surface protein in a subject in need thereof, optionally wherein the subject has cancer, an autoimmune condition, an inflammatory condition, a neurodegenerative condition, or an infectious disease; and/or for increasing an immune response in a subject, such as a subject with cancer, an autoimmune condition, an inflammatory condition, a neurodegenerative condition, or an infectious disease. In some aspects, (a) the subject has a mutation in RNF43 and the multispecific binding protein does not bind to or does not activate RNF43, or (b) the subject has a mutation in ZNRF3 and the multispecific binding protein does not bind to or does not activate ZNRF3. In some such cases, where the subject has an RNF43 or ZNRF3 mutation, the subject has a cancer in which the cancer comprises the mutation.

The present disclosure also relates to methods of treating cancer, an autoimmune condition, an inflammatory condition, a neurodegenerative condition, or an infectious disease in a subject in need thereof, comprising administering to the subject an effective amount of a multispecific binding protein or pharmaceutical composition herein. The present disclosure further relates to methods of reducing the level of a cell surface protein in a subject in need thereof, comprising administering to the subject an effective amount of a multispecific binding protein or pharmaceutical composition herein, optionally wherein the subject has cancer, an autoimmune condition, an inflammatory condition, a neurodegenerative condition, or an infectious disease. And the disclosure further relates to methods of increasing an immune response in a subject in need thereof, comprising administering to the subject an effective amount of a multispecific binding protein or pharmaceutical composition herein, optionally wherein the subject has cancer, an autoimmune condition, an inflammatory condition, a neurodegenerative condition, or an infectious disease. In some aspects, (a) the subject has a mutation in RNF43 and the multispecific binding protein does not bind to or does not activate RNF43, or (b) the subject has a mutation in ZNRF3 and the multispecific binding protein does not bind to or does not activate ZNRF3. In some such cases, where the subject has an RNF43 or ZNRF3 mutation, the subject has a cancer in which the cancer comprises the mutation. In some cases, the methods further comprise, prior to administering the multispecific binding protein, determining whether the subject has a mutation in RNF43 or ZNRF3, wherein, (a) if the subject has a mutation in RNF43, the multispecific binding protein does not bind to to or does not activate RNF43, and (b) if the subject has a mutation in ZNRF3, the multispecific binding protein does not bind to or does not activate ZNRF3.

In some aspects, multispecific binding proteins herein may be used to deplete levels of a cell surface protein on particular cell types by targeting an E3 ubiquitin ligase that is primarily expressed on those cell types. For example, the present disclosure also includes methods of inducing degradation of a cell surface protein on the surface of hematopoietic cells using a multispecific binding protein herein, wherein the transmembrane E3 ubiquitin ligase used in the multispecific protein is RNF130, RNF149, or RNF167. Additionally, the present disclosure also includes methods of inducing degradation of a cell surface protein on the surface of testicular cells using a multispecific binding protein herein, wherein the transmembrane E3 ubiquitin ligase used in the multispecific protein is RNF133 or RNF148. In certain aspects, the multispecific binding protein reduces the level of the cell surface protein on the surface of a particular cell type, such as a hematopoietic or testicular cell, in vitro compared to the level observed in the absence of the multispecific binding protein. In some such cases, the multispecific binding protein reduces the level of the cell surface protein on the surface of a particular cell type, such as a hematopoietic or testicular cell, in vitro as determined by flow cytometry or by luminescense assay. In some cases, a multispecific binding protein reduces the level of the second cell surface protein on the surface of a particular cell type, such as these, in vivo compared to the level observed in the absence of the multispecific binding protein, or alternatively, both in vitro and in vivo. Accordingly, the present disclosure also includes methods of reducing the level of a cell surface protein on the surface of hematopoietic cells in a cell or tissue sample in vitro or in vivo in a subject, comprising administering to the cell or tissue sample, or to the subject a multispecific binding protein that targets one or more of RNF130, RNF149, and RNF167. The disclosure also includes methods of reducing the level of a cell surface protein on the surface of testicular cells in a cell or tissue sample in vitro or in vivo in a subject, comprising administering to the cell or tissue sample or to the subject a multispecific binding protein that targets one or both of RNF133 and RNF148.

The present disclosure also encompasses kits comprising a multispecific binding protein, nucleic acid, set of nucleic acids, or host cell described herein, and further comprising one or more reagents for expressing or purifying the multispecific binding protein and/or one or more reagents for incubating the protein with a cell or tissue sample in vitro to reduce the level of a cell surface protein in the sample. And the disclosure also encompasses methods of reducing the level of a cell surface protein in a cell or tissue sample in vitro, comprising incubating the sample with a multispecific binding protein described herein.

### BRIEF DESCRIPTION OF THE FIGURES

FIGS. 1A-1F show that cell surface ligases driven by Oncogenic Wnt can be repurposed for targeted degradation. FIG. 1A) Schematic depicting the role of RNF43 in controlling cell surface abundance of frizzled (FZD) receptors through ubiquitination. FIG. 1B) Human adenoma primary tissues expression of RNF43 (left panel) and ZNRF3 (right panel) (see Galamb O., et al., Cancer Epidemiol Biomarkers Prev 17(10): 2835-2845, 2008). FIG. 1C) pan-TCGA (The Cancer Genome Atlas) data showing expression of RNF43 in various cancer subtypes. Note the high expression in colorectal cancer and other tumor subtypes displaying elevated Wnt activity (red dots indicate tumor tissue; black dots indicate normal tissue). FIG. 1D) Doxycycline inducible expression of wild-type (WT) RNF43, but not a mutant, delta RING RNF43 that is deficient in ligase activity, leads to decreased FZDs cell surface expression. FIG. 1E) Schematic strategy used to chemically dimerize RNF43 and/or ZNRF3 to a non-natural cell surface with an A/C heterodimerizer, including Receptor tyrosine kinases (RTKs) and G protein-coupled receptors (GPCRs). FIG. 1F) Immunoprecipitation of tagged RNF43 followed by immunoblotting of tagged HER2 in transiently transfected HEK293T cells after treatment with A/C heterodimerizer.

FIGS. 2A-2E shows antibody mediated dimerization of gD-tagged cell surface RNF43 or ZNRF3 ligases to HER2. FIG. 2A) Schematic depicts dimerization of N-terminus gD-tagged RNF43 or ZNRF3 ligases to protein of interest using an anti-gD/anti-protein of interest (POI) bispecific antibody. FIG. 2B) Bispecific antibodies against gD and three distinct HER2 epitopes. FIG. 2C) Fluorescence assisted cell sorting (FACS) plot depicting cell surface gD expression of HEK293T cells transiently transfected with either gD tagged RNF43 or ZNRF3. FIG. 2D) Western blot of HER2 in HEK293T transiently transfected with gD tagged RNF43, gD tagged ZNRF3 or control (“mock”) after treatment with bispecific antibodies targeting gD and HER2 (“gD/HER2”). Actin is used as a loading control. FIG. 2E) Western blot of HER2 in MCF7, KPL4 and SKBr3 cells stably transfected with gD-RNF43 and gD-ZNRF3 after treatment with the depicted bispecific antibodies. Downstream modulation of the HER2 pathway is depicted in SKBr3 using p-Erk. Actin is used as a loading control.

FIGS. 3A-3E show a mechanism of cell surface clearance and receptor degradation upon RNF43/ZNRF3 dimerization. FIG. 3A) Mean fluorescence intensity (MFI) of cell surface HER2 in HT29 cells transfected with gD-ZNRF3 after treatment with various antibodies against gD/HER2 or CD3/HER2. FIG. 3B) Western blot of HER2 in HEK293T transiently transfected with gD tagged ZNRF3 after treatment with the depicted bispecific antibodies against gD/HER2 or CD3/HER2 or transfected with a guide RNA driving genetic deletion of HER2. Tubulin is used as a loading control. FIG. 3C) Immunoprecipitation of HER2 followed by immunoblotting of ubiquitin in HEK293T transiently transfected with gD tagged RNF43, ZNRF3 or control (“mock”) after treatment with the depicted three different bispecific antibodies against gD and HER2: T (Trastuzumab, 4D5), P (Pertuzumab, 2C4), and 7 (7C2). FIG. 3D) Western blot of HER2 in HEK293T transiently transfected with gD tagged RNF43, ZNRF3 that are either proficient (WT RING) or deficient (delta-RING) in ligase activity, or control after treatment with the depicted bispecific antibodies against gD/HER2. Actin is used as a loading control. FIG. 3E) Western blot of HER2 in HEK293T transiently transfected with gD tagged RNF43, ZNRF3 or control after treatment with the depicted bispecific antibodies against gD/HER2 and depicted inhibitors (Bortezomib=proteasome inhibitor; E1 activating enzyme inhibitor, BafA=Lysosome inhibitor). Actin is used as a loading control. (T=Trastuzumab (4D5), P=Pertuzumab (2C4), 7=7C2)

FIGS. 4A-4F show phenotypic impact of cell surface ligase mediated receptor degradation. FIG. 4A) Immunoprecipitation of tagged RNF43 followed by immunoblotting of tagged IGF1R in transiently transfected HEK293T cells after treatment with AC heterodimerizer. Tubulin is used as a loading control. FIG. 4B) Western blot of IGF1R in HT29 cells transfected with a doxycycline (“dox”) inducible gD tagged ZNRF3 after treatment with dox and the depicted bispecific antibodies against gD an various IGF1R epitopes. Tubulin is used as a loading control. FIG. 4C) Clonogenic growth assay of HT55 cells transfected with a dox inducible CRISPR-Cas9 and sgRNA targeting IGF1R or non-targeting control (NTC) sgRNA. FIG. 4D) In vivo tumor growth of HT55 cells transfected with a dox inducible CRISPR-Cas9 and sgRNA targeting IGF1R or non-targeting control (NTC) sgRNA. FIG. 4E) Clonogenic growth assay of HT55 cells transfected with a dox inducible gD-ZNRF3 after treatment with anti-Citxu/gD or anti-Citxu/NIST bispecific antibodies. Licor based quantification of FIG. 4E) is depicted in FIG. 4F).

FIGS. 5A-5F provide a characterization of RNF43 and ZNRF3 targeted antibodies and related bispecifics. FIG. 5A) SPR binding of antibodies discovered from rat or rabbit immunizations to RNF43 or ZNRF3. FIGS. 5B and 5C) Cross-blocking analysis of a subset of ZNRF3 (FIG. 5B) and RNF43 (FIG. 5C) binding antibodies. FIG. 5D) Time course of cixutumumab/hSC37.39 bispecific antibody driving cell surface clearance of IGF-1R at zero to 10 μg/mL concentrations, measured by tracking IGF1R MFI at 1 to 24 hours. FIG. 5E and FIG. 5F) Clearance of IGF1R using a panel of ligase/cixutumumab bispecific antibodies binding to human ZNRF3 (“hu ZNRF3”; FIG. 5E) or human RNF43 (“hu RNF43”; FIG. 5F). Percent clearance is plotted versus monovalent affinity for each of the respective ligases.

FIGS. 6A-6C show characterization of the degradation potential of novel cell surface ligase antibodies. FIG. 6A and FIG. 6B) Mean fluorescence intensity (MFI) of cell surface IGF1R in SW1417 or HT29 cells transfected with gD-ZNRF3 after treatment with various antibodies against IGF1R/ZNRF3 (FIG. 6A) or IGF1R/NIST (FIG. 6B). FIG. 6C) Western blot of IGF1R in parental SW1417 cells after treatment with depicted bispecific antibodies. Actin is used as loading control.

FIGS. 7A-7F show characterization of the degradation potential of novel formats of cell surface ligase antibodies. FIG. 7A and FIG. 7B) show schematics of the tested antibodies. FIG. 7C)-FIG. 7F) show flow cytometry analysis of IGF1R to assess antibody activity in HT29 cells overexpressing the relavent ligase. FIG. 7C shows results for cixutumumab/anti-RNF43 constructs with one or two cixutumumab (anti-IGFR; “cixu”) binding regions, FIG. 7D shows results for cixutumumab/anti-RNF43 constructs with one or two RNF43 binding regions, FIG. 7E shows results for istiratumab/anti-RNF43 constructs with one or two istiratumab (anti-IGFR; “istira”) binding regions, and FIG. 7F shows results for istiratumab/anti-RNF43 constructs with one or two RNF43 binding regions.

FIGS. 8A-8D show characterization of the degradation potential of additional novel formats of cell surface ligase antibodies. FIG. 8A) Schematics of the tested FvIgG or FabIbG format antibodies are shown. FIG. 8B) Flow cytometry analysis of IGF1R is used to assess activity of the FvIgG formats in HT29 cells overexpressing the relavent ligase. FIG. 8C and FIG. 8D) Flow cytometry analysis of IGF1R is used to assess activity of the FabIgG formats in HT29 cells overexpressing the relavent ligase (FIG. 8C=cixutumumab/anti-RNF43; FIG. 8D=istiratumab/anti-RNF43).

FIG. 9 shows characterization of the degradation potential of EGFR targeted multispecifics. Flow cytometry analysis of EGFR is used to assess activity of the multispecifics in HT29 cells overexpressing the relavent ligase.

FIGS. 10A-10F show characterization of degradation potential of novel cell surface ligase antibodies. FIG. 10A) Cartoon depicting the structural domains of both RNF43 and ZNRF3, including signal peptide (SP), Transmembrane domain TM and ligase domain (RING). FIG. 10B) Identification and gD tagging of additional E3 ligases with similar structural characteristics as RNF43 and ZNRF3, including SP and TM. FIG. 10C) FACS plot depicts transfection of gD-ZNRF3 in HEK293T cells (FITC positive) and gD cell surface detection using anti-gD antibody (conjugated to APC). FIG. 10D) Cell surface MFI of gD signal from depicted gD tagged ligases transiently transfected into HEK293T cells. gD is detected using anti-gD APC conjugated antibody. FIG. 10E and FIG. 10F) Western blots of HER2 in HEK293T cells transiently transfected with the depicted dox inducible gD tagged ligases after treatment with anti-HER2/gD bispecific antibody. Western blot for gD and tubulin are used transfection evaluation and loading control, respectively. FIG. 10E shows data for ligases RNF13, RNF43, RNF128, RNF130, RNF133, RNF148, and RNF149. FIG. 10F shows data for ligases RNF150, RNF167, ZNRF3, LNX1, RSPRY1, SYVN1, and TRIM7.

FIGS. 11A and 11B show the activation of Wnt/β-catenin signaling pathway via TCF reporter by ZNRF3 antibodies. FIG. 11A) Rat clone ZNRF3 bivalent antibodies triggered minor activation of Wnt/β-catenin in the presence of 100 ng/mL recombinant Wnt3a in HEK293 cells. Cells without any treatment, with DMSO, and with 100 ng/mL Wnt3a were negative controls; cells treated with 2 μM GSK3beta or various concentration of RSPO3 (500 ng/μL, 10 ng/μL, 0.2 ng/μL) in combination with 100 ng/mL Wnt3a were positive controls. FIG. 11B) Rabbit clone ZNRF3 bivalent antibodies triggered minor activation of Wnt/β-catenin. Positive and negative controls used are described above. Data shown are from two independent experiments (n=2).

FIGS. 12A-12F show the kinetics of bispecific antibody-mediated surface IGF1R-HibiT clearance. FIG. 12A-12C) Percent cell surface IGF1R-HibiT that remained was evaluated at 0, 4, 8 and 24 hours (h) with bispecific antibodies Cixu/ZNRF3-6 (FIG. 12A), Cixu/ZNRF3-55 (FIG. 12B) and Cixu/RNF43-67 (FIG. 12C) at concentrations of 10 μg/mL and 1 μg/mL. A time-dependent clearance was observed. Cixu/RNF43 is a positive control. FIG. 12D-12F) Time-dependent clearance was observed using the new format Cixu-RNF43 Fv-IgG (FIG. 12D) and Istira-RNF43 Fv-IgG (FIG. 12E), and Istira-RNF43 Fv-IgG (FIG. 12F) at 10 μg/mL displayed a “hook effect”.

FIGS. 13A-13C show cellular toxicity and cell surface clearance evaluated by multiplexing the HiBiT extracellular detection assay with LDH or CellTiter-Glo® assay. FIG. 13A) IGF1R-HibiT remained on the cell surface after treatment with cixu/ZNRF3 bispecific antibodies at 1 μg/mL over 48 h. FIG. 13B) Lactate dehydrogenase released by HT29 cells treated with cixu/ZNRF3 bispecific antibodies at 1 μg/mL over 48 h was measured using an LDH cytotoxicity assay. No toxicity was observed with the treatment conditions. FIG. 13C) CellTiter-Glo® assay measured luminescence signal (RLU) was performed after detection of IGF1R-HiBiT on the cell surface using the HibiT extracellular detection system. Similar to the LDH assay, no toxicity was observed with the treatment conditions.

FIGS. 14A and 14B show lytic detection of the total level of IGF1R-HibiT. FIG. 14A) The HiBiT extracellular system is used to detect the remaining amount of IGF1R-HibiT on the surface of HT29 cells. The lytic detection shows a total level of IGF1R-HiBiT (extracellular and intracellular). Both systems measure a luminescence signal. Cixu-RNF43 Fv-IgG format caused 20% IGF1R-HibiT to remain on the cell surface and 80% intact IGF1R-HiBiT. Cixu/RNF43.hSC37.39 was a positive control. Cixu/NIST, Cixu/Cixu and UT were negative controls. FIG. 14B) Western-blot shows the total amount of IGF1R_HibiT or IGF1R in WT HT29 cells upon 24 h treatment of new format and bispecific antibodies. Pro IGF1R-HiBiT/IGF1R is the precursor of IGF1R-HibiT/IGF1R. B-actin is an internal control.

FIGS. 15A-15G show different, exemplary formats for constructing multispecific antibodies that are compatible with embodiments herein, a “2+1 FabIgG” format (FIG. 15A), a “one armed FvIgG” or “OA FvIgG” format (FIG. 15B), and a one armed FabIgG” or “OA FabIgG” format (FIG. 15C). FIGS. 15D-15E show two different optional versions of a trispecific anti-EGFR-RNF43-ZNRF3 antibody, while FIGS. 15F-15G show two different optional versions of a trispecific anti-IGF1R-RNF43-ZNRF3 antibody.

FIG. 16 shows that combinations of bispecifics targeting both RNF43/IGF1R and ZNRF3/IGF1R are more effective at removing IGF1R from the cell surface than either bispecific alone.

FIG. 17 shows that RNF43 is homogeneously expressed throughout the tumor mass in a transplanted murine APC mutant colorectal cancer model.

FIGS. 18A-18B show Western-blot showing the total amount of IGF1R in WT LS180 cells upon 24 h treatment of various bispecific antibodies (FIG. 18A). Pro IGF1R is the precursor of IGF1R. Tubulin is used as a loading control. Degradation percentage is summarized across various cell lines (FIG. 18B).

FIGS. 19A-19B show Western blot analysis of lysates derived from HT29 cells subjected to the indicated treatments: IGF1 stimulation (+IGF1; 50 ng/ml; 5 minutes), no treatment (−), bivalent antibody treatment (Cixu; 1 μg/ml; 135 minutes), control bispecific antibody (NIST; 1 μg/ml; 135 minutes), RNF43-based bispecific antibody (RNF43-35; 1 μg/ml; 135 minutes) or ZNRF3-based bispecific antibody (ZNRF3-55; 1 μg/ml; 135 minutes) following IgG or IGF1RP immunoprecipitation (IP). IGF1R ubiquitylation was evaluated by immunoblotting IP samples using an antibody against ubiquitin. Immunoprecipitated and total IGF1Rβ levels were evaluated using an antibody against IGF1Rβ and α-TUBULIN was used as a loading control (FIG. 19 A). Western blot analysis of lysates derived from parental HEK293T cells or HEK293T cells expressing N-terminally gD and C-terminally FLAG tagged ZNRF3 wild type (WT) or the delta RING mutant (ΔRING) following incubation with HA epitope tag antibody agarose conjugate or agarose-TUBE2. Total and co-precipitated IGF1R levels were evaluated by immunoblotting using an antibody against IGF1RP. ZNRF3 ubiquitylation and expression levels were evaluated using an antibody against the C-terminal FLAG tag and α-TUBULIN was used as a loading control (FIG. 19B).

FIG. 20 shows Western blot of IGF1R in HEK293T transiently transfected with gD tagged ZNRF3 that is either proficient (WT RING) or deficient (delta-RING) in ligase activity, or control after treatment with the depicted bispecific antibodies against gD/Cixutumumab. Tubulin is used as a loading control.

FIGS. 21A-21C show distribution of RNF43 expression is plotted against the presence of RNF43 mutations. Particular emphasis is made on the SW48 CRC line that display elevated RNF43 expression and a frameshit variant within the RING domain (FIG. 21A). Cell surface expression of RNF43 is showed for a the non expressing line RKO compared to the RING domain mutant SW48 (FIG. 21 B). Western blot of IGF1R in SW48 after treatment with the depicted bispecific antibodies against RNF43/IGF1R or ZNRF3/IGF1R (FIG. 21 C).

FIGS. 22A-22C show schematic representation of indels generation within RNF43 (FIG. 22A) and ZNRF3 (FIG. 22 B) using CRISPR CAS9 system. FACS plot depicts cell surface ligase expression of RNF43 or ZNRF3 in the presence of n-terminal truncation (N-term) or RING indel. Western blot of IGF1R in HT29 cells that are ligase proficient, N-term knock out or RING deficient transiently transfected with gD tagged ZNRF3 that is either proficient after treatment with the depicted bispecific antibodies (FIG. 22 C).

FIG. 23 shows a Western blot of IGF1R in DLD1 after treatment with the depicted bispecific antibodies against gD/IGF1R and depicted inhibitors (Bortezomib=proteasome inhibitor; E1 activating enzyme inhibitor, BafA=Lysosome inhibitor). Tubulin is used as a loading control, Ubiquitin is used to validate inhibition of the proteasome and E1 activating enzyme. LC3B is used to confirm the inhibition of the lysosome.

FIG. 24 shows a Western blot of IGF1R in DLD1 upon 24 h treatment of new format and bispecific antibodies show three different, exemplary formats for constructing bispecific antibodies that are compatible with embodiments herein, a “2+1 FabIgG” format (FIG. 15A), a “one armed FvIgG” or “OA FvIgG” format.

FIGS. 25A-25C show Western blot of IGF1R, pIGF1R and downstream component of the IGF1R signaling axis, including pAKT and pS6 in SW48 cells treated with various depicted antibodies (FIG. 25A). Note the near complete inhibition of pAKT signaling upon ligase mediated degradation of IGF1R. Clonogenic outgrowth of SW48 cells 14 days after treatment with depicted antibodies (FIG. 25B) and licor based quantification of (FIG. 25B) is depicted in (FIG. 25C).

FIGS. 26A-26D show a lonogenic growth assay of HT29 cells transfected with a dox inducible gD-ZNRF3 after treatment with various anti-Citxu/ZNRF3 or control bispecific antibodies. Licor based quantification of FIG. 26A (results for gD-ZNRF3 WT-FLAG) and FIG. 26C (results for gD-ZNRF3 delta-RING-FLAG mutant) are depicted in FIG. 26B and FIG. 26D, respectively.

FIG. 27 shows a Western blot of PD-L1, in SW48 cells treated with various depicted antibodies. Note the deep degradation seen when ZNRF3/PD-L1 antibodies are used.

FIGS. 28A-28B show characterization of degradation potential of novel cell surface ligase antibodies. FACS plot depicts cell surface expression of gD-RNF43, RNF13 and RNF128 stable HT29 cells (FIG. 28 A). gD cell surface detection using anti-gD antibody (conjugated to APC). FIG. 28 B: Cell surface MFI of gD signal from depicted gD tagged ligases stably integrated into HT29 cells. gD is detected using anti-gD APC conjugated antibody.

FIG. 29 shows a Western blot of IGF1R in HT29 cells with the depicted dox inducible gD tagged ligases after treatment with anti-IGF 1R/gD bispecific antibody. Western blot for gD and tubulin are used transfection evaluation and loading control, respectively.

FIG. 30 shows that bispecific antibodies designed to tether endogenous RNF43 or ZNRF3 to IGF1R induce IGF1R target degradation in an SW48 in vivo xenograft model.

FIG. 31 shows a heatmap depicting expression of indicated cell surface ligases across normal tissues (source GTEX) as described in Example 18. Data is z-scored normalized.

FIG. 32 shows Western blot analysis of lysates from doxycycline treated HT29 cells harboring the indicated doxycycline inducible gD-ligase-FLAG expression constructs following 24 hours incubation with gD*IGF1R (Cixu) bispecific PROTAB. Endogenous IGF1Rβ and exogenous gD-ligase-FLAG protein levels were detected. Data are representative of three independent experiments.

FIG. 33 shows further Western blot analysis of lysates from doxycycline treated HT29 cells harboring the indicated doxycycline inducible gD-ligase-FLAG expression constructs following 24 hours incubation with gD*IGF1R (Cixu) bispecific PROTAB. Endogenous IGF1Rβ and exogenous gD-ligase-FLAG protein levels were detected. Data are representative of three independent experiments.

FIG. 34 shows Western blot analysis of lysates from doxycycline treated HT29 cells harboring the indicated doxycycline inducible gD-ligase-FLAG expression constructs following 24 hours incubation with gD*HER2 (4D5) bispecific PROTAB. Endogenous HER2 and exogenous gD-ligase-FLAG protein levels were detected. Data are representative of two independent experiments.

FIG. 35 shows Western blot analysis of lysates from doxycycline treated HT29 cells harboring the indicated doxycycline inducible gD-ligase-FLAG expression constructs following 24 hours incubation with gD*PD-L1 (Atezo) bispecific PROTAB. Endogenous PD-L1 and exogenous gD-ligase-FLAG protein levels were detected. Data are representative of two independent experiments.

FIG. 36 shows further Western blot analysis of lysates from doxycycline treated HT29 cells harboring the indicated doxycycline inducible gD-ligase-FLAG expression constructs following 24 hours incubation with gD*HER2 (4D5) bispecific PROTAB. Endogenous HER2 and exogenous gD-ligase-FLAG protein levels were detected. Data are representative of two independent experiments.

FIG. 37 shows further Western blot analysis of lysates from doxycycline treated HT29 cells harboring the indicated doxycycline inducible gD-ligase-FLAG expression constructs following 24 hours incubation with gD*PD-L1 (Atezo) bispecific PROTAB. Endogenous PD-L1 and exogenous gD-ligase-FLAG protein levels were detected. Data are representative of two independent experiments.

FIGS. 38A-38L show cell surface clearance of EpCAM (FIGS. 38A-38F) and degradation of EpCAM in doxycycline treated HT29 cells harboring the indicated doxycycline inducible gD-ligase expression constructs (FIGS. 38G-38J) following 24 hours incubation with 1 μg/ml and 10 μg/ml of the gD-EpCAM or control gD-NIST bispecific PROTAB. FIG. 38A-FIG. 38F show % EpCAM clearance assessed by FACS, while FIG. 38G-FIG. 38L show EpCAM degradation assessed by MFI. FIG. 38A and FIG. 38G: HT29 cells; FIG. 38B and FIG. 38H: HT29-gD-RNF43; FIG. 38C and FIG. 38I: HT29-gD-RNF133; FIG. 38D and FIG. 38J HT29-gD-RNF149; FIG. 38E and FIG. 38K: HT29-gD-RNF150; and FIG. 38F and FIG. 38L: HT29-gD-ZNRF3. FIG. 38C and FIG. 38D show that HT29-gD-RNF133 and HT29-gD-RNF149 had the highest clearance of EpCAM (20-30%) compared to the other HT-29-gD-ligases, while FIG. 38I and FIG. 38J show that HT29-gD-RNF133 and HT29-gD-RNF149 had the highest degradation of EpCAM compared to the other HT29-gD-ligases. Similar results showing highest clearance and degradation of HER2, EGFR, and IGF1R by the respective gD-HER2, gD-EGFR, or gD-IGF1R PROTAB in HT29-gD-RNF133 and HT29-gD-RNF149 compared to the other HT-29-gD-ligases were observed (data not shown).

FIGS. 39A and 39B show HER2 degradation in SW48 following bivalent or PROTAB antibody treatment. FIG. 39A shows level of HER2 degradation by Western blot analysis in SW48 cells left untreated (−), or subjected to a HER2 bivalent antibody (7C2), a NIST*HER2 (NIST) control bispecific antibody, or HER2 bispecific PROTABs (ZNRF3-6 and RNF43-37.39) for 48 hours. FIG. 39B shows level of HER2 degradation by Western blot analysis in SW48 cells left untreated (−), or subjected to a HER2 bivalent antibody (4D5), NIST*HER2 bispecific antibody (NIST), a ZNRF3*HER2 PROTAB antibody (ZNRF3-6), or a ZNRF3*NIST control bispecific antibody for 48 hours. The level of alpha-tubulin was used as a loading control. Data represent two independent experiments.

### DETAILED DESCRIPTION OF THE INVENTION

### I. Definitions

As utilized in accordance with the present disclosure, the following terms, unless otherwise indicated, shall be understood to have the following meanings:

A “transmembrane E3 ubiquitin ligase” refers to a class of protein that comprises at least one cell membrane spanning region and that has E3 ubiquitin ligase activity. A transmembrane E3 ubiquitin ligase has “catalytic activity” herein if it is capable of facilitating the final transfer of ubiquitin from the ubiquitin-conjugating enzymes (E2s) to substrates to alter that substrate function. In some embodiments, catalytic activity may be determined in a cell surface assay (see, e.g., Example 18 below.)

A “protein of interest” or a “protein targeted for degradation” or a “degradation target protein” or the like refer to a protein that is intended to be brought to lysosomes for degradation by the molecules described herein. In some embodiments herein, such a protein intended for degradation is a “cell surface protein.” A “cell surface protein” as used herein broadly refers to a protein that is located at the cell surface, either as a transmembrane protein possessing an extracellular domain, or as a protein that is otherwise localized to the cell surface, such as a protein that is bound to a transmembrane protein.

Proteins such as transmembrane E3 ubiquitin ligases and degradation targets herein may be from any vertebrate source, including mammals such as primates (e.g., humans) and rodents (e.g., mice and rats), or domestic mammals (e.g., dogs, cats, horses, livestock such as cattle, pigs, sheep, goats, etc.), avians (e.g., foul, chickens, turkey), or fish, unless otherwise indicated.

A “multispecific binding protein” as used herein refers to a protein molecule that may be used to bind to a transmembrane E3 ubiquitin ligase and also to a degradation target protein. The protein is “multispecific” because it binds to at least two target proteins (i.e., the transmembrane E3 ubiquitin ligase and the degradation target). In some embodiments, the binding protein is an antibody, such as a bispecific or multispecific antibody, or is a bispecific or multispecific protein that comprises an antibody or antibody fragment and optionally another specific protein binding domain.

The term “antibody” herein refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term is used in the broadest sense and encompasses various antibody structures, including but not limited to monoclonal antibodies, polyclonal antibodies, multispecific antibodies (e.g., bispecific antibodies, diabodies, etc.), full length antibodies, single-chain antibodies, antibody conjugates, and antibody fragments, so long as they exhibit the desired binding activity.

An “isolated” antibody is one that has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

An “antigen” refers to the target of an antibody, i.e., the molecule to which the antibody specifically binds. The term “epitope” denotes the site on an antigen, either proteinaceous or non-proteinaceous, to which an antibody binds. Epitopes on a protein can be formed both from contiguous amino acid stretches (linear epitope) or comprise non-contiguous amino acids (conformational epitope), e.g., coming in spatial proximity due to the folding of the antigen, i.e. by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein.

In this disclosure, “binds” or “binding” or “specific binding” and similar terms, when referring to a protein and its ligand or an antibody and its antigen target for example, means that the binding affinity is sufficiently strong that the interaction between the members of the binding pair cannot be due to random molecular associations (i.e. “nonspecific binding”). Such binding typically requires a dissociation constant (K_{D}) of 1 μM or less, and may often involve a K_{D} of 100 nM or less.

An “anti-RNF43 antibody” or a “RNF43-antibody” or an “antibody that specifically binds RNF43” or an “antibody that binds to RNF43” and similar phrases (e.g., an anti-gD antibody or an anti-ZNRF3 antibody, or an anti-HER2 antibody and the like) refer to an antibody that specifically binds to the indicated protein.

The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence.

The term “hypervariable region” or “HVR” as used herein refers to each of the regions of an antibody variable region which are hypervariable in sequence and which determine antigen binding specificity, for example “complementarity determining regions” (“CDRs”). Generally, antibodies comprise six CDRs: three in the VH (CDR-H1 or heavy chain CDR1, CDR-H2, CDR-H3), and three in the VL (CDR-L1, CDR-L2, CDR-L3). Exemplary CDRs herein include:
(a) “Chothia CDRs”: hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) “Kabat CDRs”: CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) “McCallum CDRs”: antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless specifically indicated, the CDRs are determined according to Kabat et al., supra. One of skill in the art will understand that the CDR designations can also be determined according to McCallum, or any other scientifically accepted nomenclature system.

“Framework” or “FR” refers to the residues of the variable region residues that are not part of the complementary determining regions (CDRs). The FR of a variable region generally consists of four FRs: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4. An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

The term “variable region” or “variable domain” interchangeably refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). See, e.g., Kindt et al. Kuby Immunology, 6^{th} ed., W.H. Freeman and Co., page 91 (2007). A variable domain may comprise heavy chain (HC) CDR1-FR2-CDR2-FR3-CDR3 with or without all or a portion of FR1 and/or FR4; and light chain (LC) CDR1-FR2-CDR2-FR3-CDR3 with or without all or a portion of FR1 and/or FR4. That is, a variable domain may lack a portion of FR1 and/or FR4 so long as it retains antigen-binding activity. A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The light chain and heavy chain “constant regions” of an antibody refer to additional sequence portions outside of the FRs and CDRs and variable regions. Certain antibody fragments may lack all or some of the constant regions. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “Fe region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to EU index). Therefore, the C-terminal lysine (Lys447), or the C-terminal glycine (Gly446) and lysine (Lys447), of the Fc region may or may not be present. Thus, a “full-length IgG1” for example, includes an IgG1 with Gly446 and Lys447, or without Lys447, or without both Gly446 and Lys447. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, may comprise Gly446 and Lys447 (numbering according to EU index). In one aspect, a heavy chain including an Fc region as specified herein, comprised in an antibody according to the invention, may comprise Gly446 (numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. In certain aspects, the antibody is of the human IgG1 IgG2, IgG3, or IgG4 isotype. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen (i.e. RNF43, ZNRF3, gD, or a degradation target protein) to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The terms “full length antibody”, “intact antibody”, and “whole antibody” are used herein interchangeably to refer to an antibody having a structure substantially similar to a native antibody structure or, in the case of an IgG antibody, having heavy chains that contain an Fc region as defined herein.

The term “multispecific” herein refers to a molecule that can bind to more than one different target or antigen, such as to two or three or more different targets or antigens. The term “bispecific” herein refers to a molecule such as a binding protein or antibody that is able to specifically bind to two different targets or antigens. A “multispecific” or “bispecific” antibody herein may include the appropriate full length heavy and light chains for binding to two different antigens, or it may include appropriate antibody fragments for binding to two different antigens. There are a variety of different platforms for creating multispecific binding proteins that are compatible with this disclosure.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric” antibody refers to an antibody in which a portion of the heavy and/or light chain is derived from a particular source or species, while the remainder of the heavy and/or light chain is derived from a different source or species.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

An “immunoconjugate” is an antibody conjugated to one or more heterologous molecule(s), including but not limited to a cytotoxic agent. A “naked antibody” refers to an antibody that is not conjugated to a heterologous moiety (e.g., a cytotoxic moiety) or radiolabel. The naked antibody may be present in a pharmaceutical composition.

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody” refers to one or more nucleic acid molecules encoding antibody heavy and light chains of antibodies herein (or fragments thereof), including such nucleic acid molecule(s) in a single vector or separate vectors, and such nucleic acid molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R.

Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

By “reduce” is meant the ability to cause an overall decrease of 20%, 30%, 40%, 50%, 60%, 70%, 75%, 80%, 85%, 90%, 95%, or greater. In some embodiments, reduce or inhibit can refer to a relative reduction compared to a reference (e.g., reference level of biological activity (e.g., wnt signaling) or binding). In some embodiments, reduce may refer to reduction of the “level” (i.e. the amount or concentration) of a cell surface protein on a cell, for example. Degradation of a protein, for example, can result in reduction of the level of that protein observed in a cell or tissue sample, such as by flow cytometry or by qualitative analysis of fluorescence staining.

A multispecific binding protein that “blocks binding of” a transmembrane E3 ubiquitin ligase to a ligand refers to the ability to inhibit the interaction between the ligase and one of its ligands. For example, ligases such as RNF43 and ZNRF3 bind to native ligands such as frizzled (FZD) and LRP6. In some aspects, a multispecific binding protein does not block such binding. Such inhibition may occur through any mechanism, including direct interference with ligand binding, e.g., because of overlapping binding sites on the ligase for the antibody and one or more ligands, and/or indirect interference with ligand binding, such as allosteric interference with binding, e.g., by causing conformational changes in the ligase that alter ligand affinity.

As used herein, the term “about” refers to a numeric value, including, for example, whole numbers, fractions, and percentages, whether or not explicitly indicated. The term “about” generally refers to a range of numerical values (e.g., +/−5-10% of the recited range) that one of ordinary skill in the art would consider equivalent to the recited value (e.g., having the same function or result). When terms such as “at least” and “about” precede a list of numerical values or ranges, the terms modify all of the values or ranges provided in the list. In some instances, the term about may include numerical values that are rounded to the nearest significant figure.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

Exemplary techniques used in connection with recombinant DNA, oligonucleotide synthesis, tissue culture and transformation (e.g., electroporation, lipofection), enzymatic reactions, and purification techniques are described, e.g., in Sambrook et al. Molecular Cloning: A Laboratory Manual (2nd ed., Cold Spring Harbor Laboratory Press, Cold Spring Harbor, N.Y. (1989)), among other places.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

An “individual” or “subject” is a human unless otherwise specified. In some cases, where specified, an “individual” or “subject” is a non-human mammal or includes non-human mammals (e.g. “a mammalian subject” or a “non-human mammal subject”). Mammals include, but are not limited to, domesticated animals (e.g., cows, sheep, cats, dogs, and horses), primates (e.g., humans and non-human primates such as monkeys), rabbits, and rodents (e.g., mice and rats). In some cases, non-mammalian animals whose cells express transmembrane E3 ubiquitin ligases can also be specified (e.g., avians or fish).

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

An “effective amount” of an agent, e.g., a pharmaceutical composition, refers to an amount effective, at dosages and for periods of time necessary, to achieve the desired therapeutic or prophylactic result.

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

### II. Multispecific Binding Proteins

In one aspect, the disclosure herein concerns multispecific binding proteins that are capable of specifically binding both at least one first cell surface protein which comprises a transmembrane E3 ubiquitin ligase and also to at least one second cell surface protein. In some embodiments, the second cell surface protein may be intended for degradation (i.e. a degradation target protein). The multispecific binding protein molecules may have a variety of general formats. For example, multispecific binding proteins, in some embodiments, may be bispecific (i.e. binding to two targets) or in some embodiments, may be able to bind to more than two targets or to more than one site on a target. In some embodiments, multispecific antibodies may be trispecific, i.e., binding to three targets or sites. In other embodiments, they may bind to more than three targets or sites. In some embodiments, multispecific binding proteins are multispecific antibodies, including multispecific antibody fragments as well as multispecific full length antibodies such as full length IgG antibodies. There are various bispecific and trispecific and other multispecific antibody formats compatible with molecules herein, as described below.

In other embodiments, multispecific binding proteins herein may comprise other types of specific ligands for the target molecules or a combination of an antibody variable region specific for one target and a different type of protein ligand specific for another target. In some embodiments, the multispecific binding proteins herein are known by the acronym PROTAB.

In some embodiments, the transmembrane E3 ubiquitin ligase is any one of RNF43, ZNRF3, RNF13, RNF128, RNF130, RNF133, RNF148, RNF149, RNF150, RNF167, ZNRF4, RSPRY1, SYVN1, LNX1 isoform 2, or TRIM7 isoform 3. In some embodiments, the transmembrane E3 ubiquitin ligase is a RNF13, RNF43, SYVN1, RNF130, RNF148, RNF149, LNX1_isoform 2, RNF128, RNF133, ZNRF4, RSPRY1, TRIM7_isoform 3, RNF167, RNF150, or ZNRF3 comprising an amino acid sequence selected from any one of SEQ ID Nos: 445-459, respectively. (See the sequence table below.) In some embodiments, the transmembrane E3 ubiquitin ligase is RNF43, RNF128, RNF130, RNF133 RNF149, RNF150, or ZNRF3. In some embodiments, the transmembrane E3 ubiquitin ligase is RNF130, RNF133, RNF149, or RNF150. In some embodiments, the transmembrane E3 ubiquitin ligase is RNF130, RNF149, or RNF167. In some embodiments, the transmembrane E3 ubiquitin ligase is RNF133 or RNF148. In some embodiments, the transmembrane E3 ubiquitin ligase is RNF43 or ZNRF3. In some embodiments, the multispecific binding protein binds to one type of transmembrane E3 ubiquitin ligase, e.g., ZNRF3 or RNF43. In other embodiments, the multispecific binding protein binds to two transmembrane E3 ubiquitin ligases, such as to both RNF43 and ZNRF3. For example, the multispecific binding protein may comprise two different antibody fragments, one recognizing RNF43 and one recognizing ZNRF3, allowing for binding to both ligases.

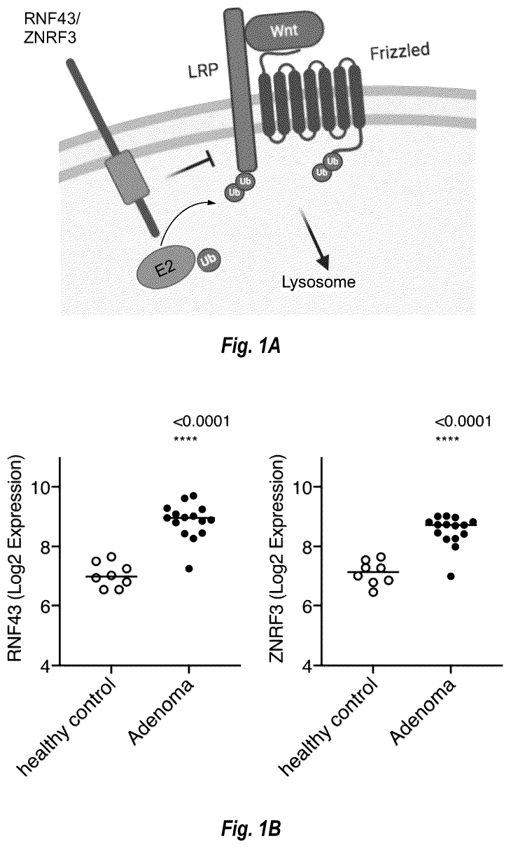

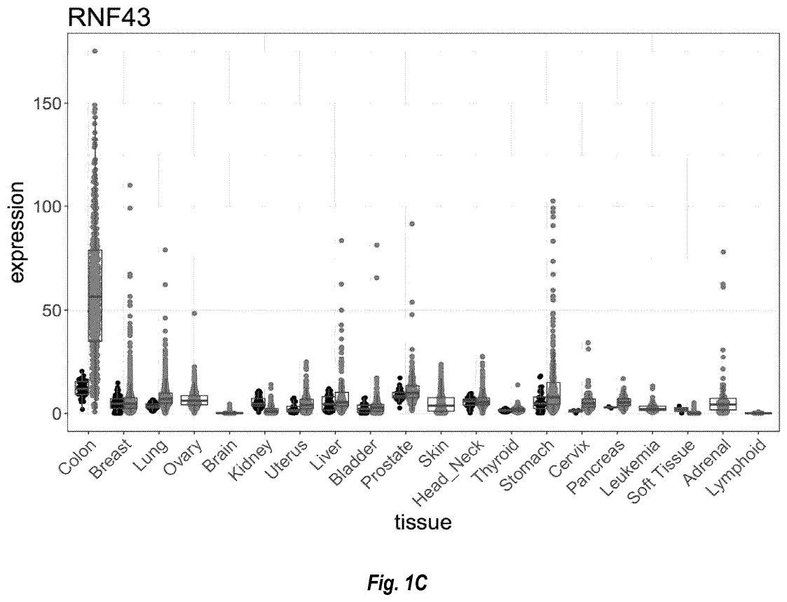

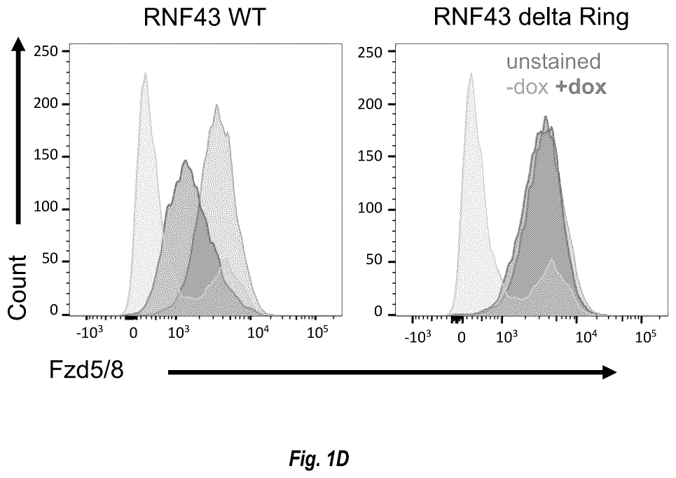

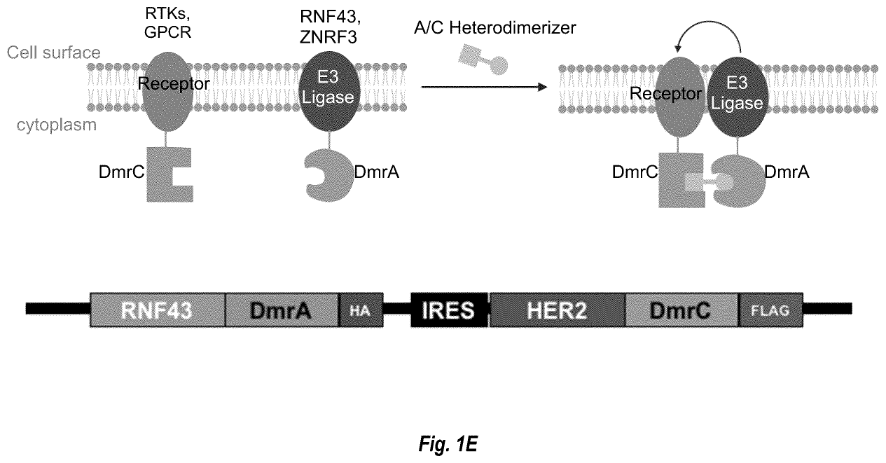

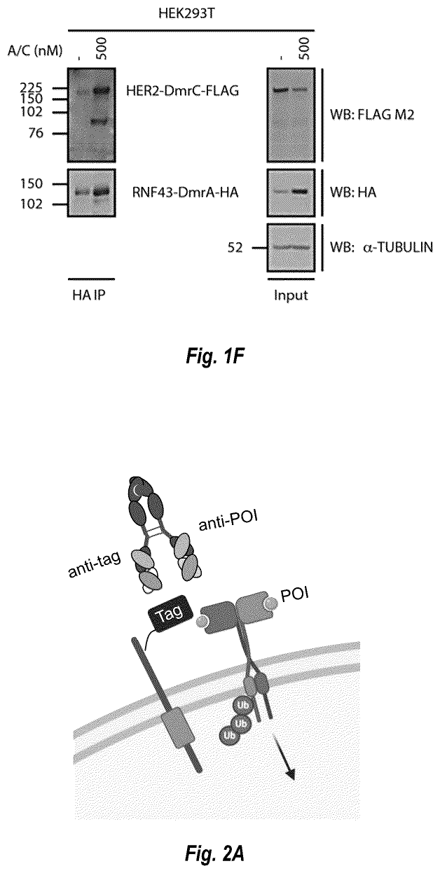

As shown in FIG. 1C, for example, RNF43 is expressed in a number of cancer cell lines. And both RNF43 and ZNRF3 are members of the Wnt signaling pathway, which is activated in a number of tumor cell lines. As those proteins are expected to be expressed on numerous types of tumors, constructs herein may be particularly useful in reducing the level of one or more target cell surface proteins in tumors in a subject. For example, constructs herein may be useful in reducing the level of target cell surface proteins wherein high levels of such proteins are known to promote cancer growth. In addition, certain target cells, such as cancer cells, may express high levels of a transmembrane E3 ubiquitin ligase like RNF43 or ZNRF3, which may allow the multispecific binding proteins to preferentially bind to those target cells in comparison to other normal or nondiseased cells that express lower levels of the ligase. As such, constructs herein may be useful in treatment of cancers. And, depending on the cell surface protein chosen for degradation, constructs herein may be useful in increasing an immune response in a subject, such as in a cancer subject.

The disclosure herein also relates to exemplary anti-RNF43 and anti-ZNRF3 antibodies that may be useful in constructing multispecific binding proteins herein, such as multispecific or bispecific antibodies. These are described in more detail below.

In some embodiments, multispecific binding proteins may also block the ability of the normal ligands for the transmembrane E3 ubiquitin ligase to bind to it. Thus, for example, in some embodiments, the proteins may block the ability of FZD or LRP6 to bind to RNF43 or ZNRF3. In other embodiments, the multispecific binding proteins do not block binding of the transmembrane E3 ubiquitin ligase to its endogenous ligands.

In some embodiments, the multispecific binding protein reduces the level of the cell surface protein on the surface of a cell compared to the level observed in the absence of the multispecific binding protein. In some cases, the multispecific binding protein reduces the level of the cell surface protein on the surface of a cell in vitro, such as observed by flow cytometry, compared to the level observed in the absence of the multispecific binding protein. In some cases, the multispecific binding protein reduces the level of the cell surface protein on the surface of a cell in vivo compared to the level observed in the absence of the multispecific binding protein.

In addition to the transmembrane E3 ubiquitin ligases, multispecific binding proteins of the disclosure may recognize a second cell surface protein, such as a protein that may be targeted for degradation by being brought into close proximity with the transmembrane E3 ubiquitin ligase.

There are a wide variety of cell surface proteins that can be targeted for degradation by the instant multispecific binding proteins. Examples include, for instance, receptor tyrosine kinases, growth factor receptors, cytokines including cytokine receptors, mucins, Siglec receptors, and immune checkpoint modulators. Examples of growth factor receptors include, for example, fibroblast growth factor receptors FGFRs, vascular endothelial growth factor receptors VEGFRs, epidermal growth factor receptors EGFRs, and platelet derived growth factor receptors PDGFRs. Various growth factor receptors can be overexpressed in certain cancers, for example. Examples of mucins include, for example, MUC1, MUC2, MUC3A, MUC3B, MUC4, MUC5B, MUC6, MUC7, MUC8, MUC12, MUC13, MUC15, MUC16, MUC17, MUC19, and MUC20 as well as MUC21 and MUC22. Mucins such as MUC1 may be overexpressed in certain cancers. Examples of Siglec (sialic acid binding immunoglobulin type lectins) receptors include, for example, CD22, CD33, MAG/Siglec-4, Siglec-5, Siglec-7, and Siglec-9. Exemplary cytokine receptors include, for example, members of the TNFR super family such as CD40, CD27, OX40, 4-1BB, and others. Particular example cell surface proteins that could be targeted by multispecific binding proteins herein include, for example, HER2, HER3, IGF1R, an EGFR, an FGFR, a VEGFR, a PDGFR, EpCAM, FZD, PD-L1, CTLA4, PD-1, TIM3, LAG3, TIGIT, CEACAM1, CD25, ILT-2, ILT-3, ILT-4, ILT-5, LAIR-1, PECAM-1 (CD31), PILR-alpha, STRL-1, or SIRP-alpha. Specific example cell surface proteins that could be targeted by multispecific binding proteins herein include, for example, HER2, IGF1R, EGFR, FZLD5, EpCAM, and PD-L1. Depending on the particular target cell surface protein chosen, multispecific binding proteins herein may include antibodies or antibody fragments derived from, for example, an anti-HER2 antibody such as 4D5, 7C2, or 2C4, or an anti-IGF1R antibody such as cixutumumab, ganitumab, dalotuzumab, figitumumab, robatumumab, teprotumumab, or istiratumab.

While reducing the cell surface level of these or other proteins may be useful, for example, in therapeutic treatments, such as for cancer, the present multispecific binding proteins are useful in a wide variety of settings, indeed wherever there is a need to reduce the level of a particular protein at the cell surface by inducing its degradation. For example, selectively reducing the level of a particular cell surface protein in an in vitro cell culture or tissue sample may be useful in a variety of experimental settings where, for example, the impact of that protein on a particular mechanism is to be studied. Accordingly, the cell surface protein targeted for destruction may include a wide variety of cell surface proteins.

### A. Exemplary Multispecific Binding Protein Formats

In certain aspects, a multispecific binding protein provided herein is a multispecific antibody, e.g., a bispecific antibody or trispecific antibody. In other aspects, the multispecific binding protein may comprise one or more antigen binding fragments from an antibody coupled with other protein domains, such as ligand binding domains from a non-antibody protein. “Multispecific antibodies” generally are monoclonal antibodies that have binding specificities for at least two different sites, i.e., different epitopes on different antigens or different epitopes on the same antigen. In certain aspects, the multispecific antibody has three or more binding specificities. Multispecific antibodies may be prepared as full length antibodies or antibody fragments. A multispecific molecule that binds to two sites or targets is “bispecific” while one that binds to three sites or targets is “trispecific.” Thus, a bispecific binding protein, such as a bispecific antibody, herein binds to both a transmembrane E3 ubiquitin ligase and to a cell surface protein. A trispecific binding protein, such as a trispecific antibody, may be constructed to bind, for example, to a cell surface protein and more than one transmembrane E3 ubiquitin ligase, to more than one cell surface protein and to one transmembrane E3 ubiquitin ligase, or to one cell surface protein and one transmembrane E3 ubiquitin ligase but to two sites on either the cell surface protein or the ligase.

For instance, in some aspects, a multispecific binding protein herein may be engineered to bind to RNF43 and to one or more second cell surface proteins. In other aspects, the protein may be engineered to bind to ZNRF3 and to one or more second cell surface proteins. Or the protein may be engineered to bind to both RNF43 and to ZNRF3 and to one or more second cell surface proteins. In yet other aspects, the multispecific binding protein herein may be engineered to bind to RNF13, RNF128, RNF130, RNF133, RNF148, RNF149, RNF150, RNF167, ZNRF4, RSPRY1, SYVN1, LNX1 isoform 2, or TRIM7 isoform 3, and to one or more second cell surface proteins.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)).

Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting FAb” or “DAF” comprising an antigen binding site that binds to the cell surface protein and another that binds to the transmembrane E3 ubiquitin ligase (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

A particular type of multispecific antibodies, also included herein, are bispecific antibodies designed to simultaneously bind to a surface antigen on a target cell, e.g., a tumor cell, and to another cell surface protein that can target the surface antigen for lysosomal degradation. Hence, in certain aspects, an antibody provided herein is a multispecific antibody, particularly a bispecific antibody, wherein one of the binding specificities is for the cell surface protein to be degraded and the other binding specificity is for the transmembrane E3 ubiquitin ligase.

Examples of bispecific antibody formats that may be useful for this purpose include, but are not limited to, molecules wherein two scFv molecules are fused by a flexible linker (see, e.g., WO 2004/106381, WO 2005/061547, WO 2007/042261, and WO 2008/119567, Nagorsen and Bauerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular bispecific antibody formats included herein are described in WO 2013/026833, WO 2013/026839, WO 2016/020309; Bacac et al., Oncoimmunology 5(8) (2016) e1203498. Bispecific antibody formats described in the aforementioned documents that bind T cells (e.g., by binding CD3, an invariant component of the T cell receptor complex), can be adapted to bind the targets as described herein instead of T cells, e.g., by replacing the antibody fragment that binds CD3 with an antibody fragment that binds a cell surface ligase.

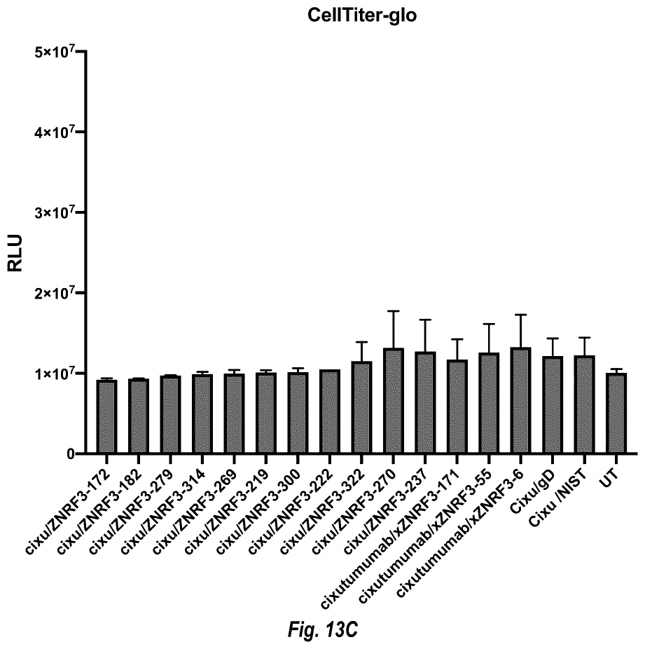

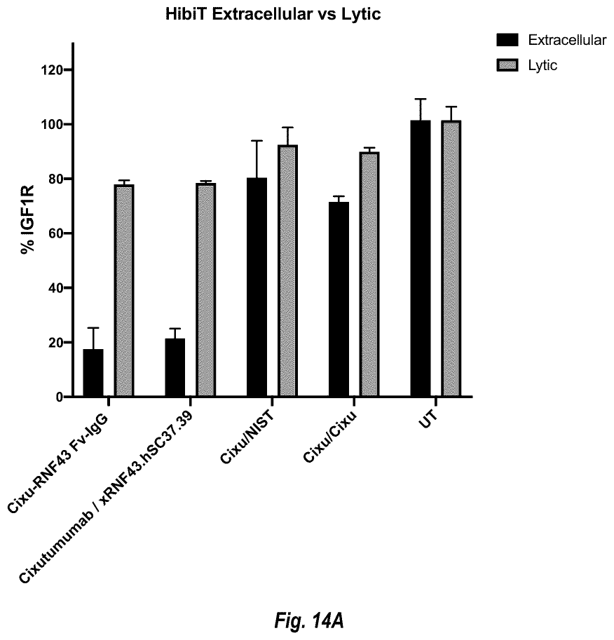

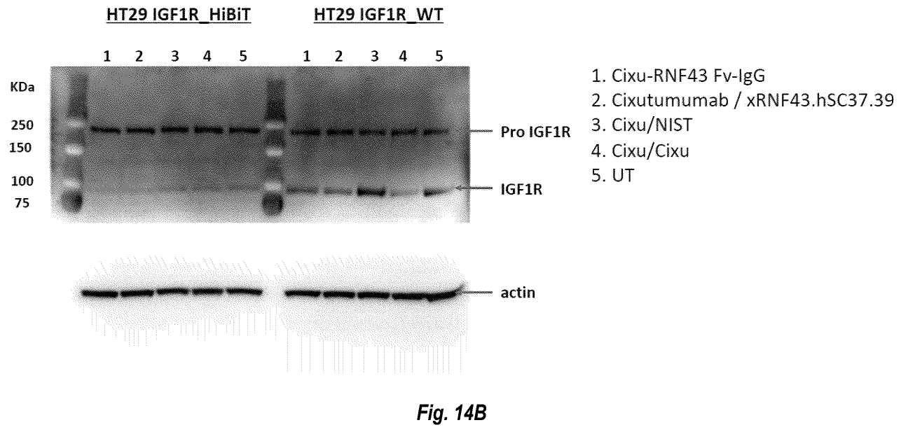

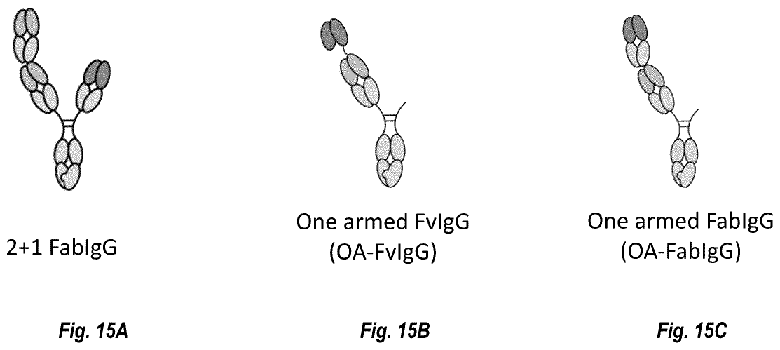

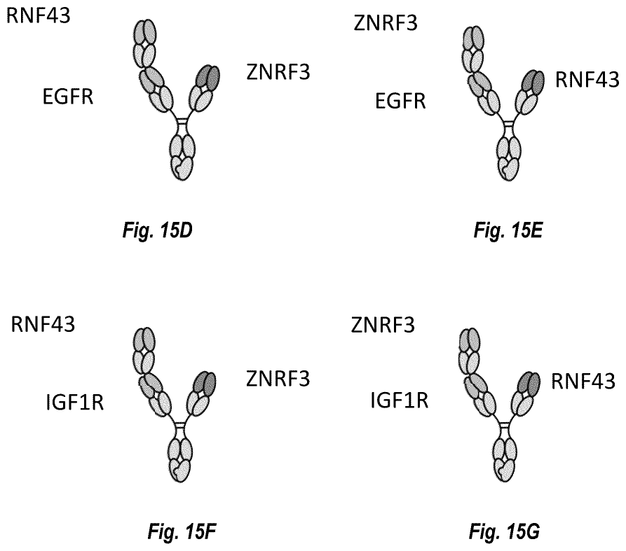

In some embodiments, a multispecific antibody herein may have a symmetrical “1+1 FabIgG” or “1+1 FvIgG” bispecific format, comprising two Fv domains recognizing different targets linked to interacting Fc regions, or comprising two Fab regions recognizing different targets linked to interacting Fc regions. Some further examples of bispecific antibody formats herein include those provided in FIGS. 15A-15C: 2+1 FabIgG, one-armed FvIgG, and one-armed FabIgG, respectively. These formats are asymmetrical, for example. Illustrations of trispecific antibodies that bind to a cell surface protein target as well as to both RNF43 and ZNFR3 are depicted in FIGS. 15D-15G. The 2+1 FabIgG format shown in FIG. 15A is described, for example, in WO 2015/095539. Optionally, antibodies such as those above comprise interacting Fc regions comprising at least one “knob-into-hole” modification. For example, a knob-into-hole modification may help to prevent mis-pairing of the Fc regions. Exemplary knob-into-hole modifications include, for instance, substitutions of one or more amino acids on a CH3 or CH2 region on one Fc domain for a larger amino acid than naturally found at that position, along with substitutions of one or more amino acids in close proximity on the partner Fc domain for a smaller amino acid than normally found. (See, e.g., U.S. Pat. No. 5,731,168, and Atwell et al., J. Mol. Biol. 270:26 (1997), as well as WO 2009/089004, WO 2012/106587, and U.S. Patent Publication 2009/0182127, for examples). In some embodiments, knob-into-hole modifications may substitutions at amino acid positions 366, 368, and 407 of a human IgG1 Fc, for example. For example, in some embodiments, “knob mutations” may comprise a substitution of T366 in a human IgG1 Fc with a larger amino acid such as W (and optionally one of S354C or Y349C) and an accompanying “hole mutation” on the partner Fc may comprise substitution of T366, L368, and Y407 for smaller amino acids such as T366S, L368A and Y407V (and optionally Y349C or S354C). (See, e.g., Carter, P. et al., Immunotechnol. 2 (1996) 73).) Numbering is according to EU index numbering.

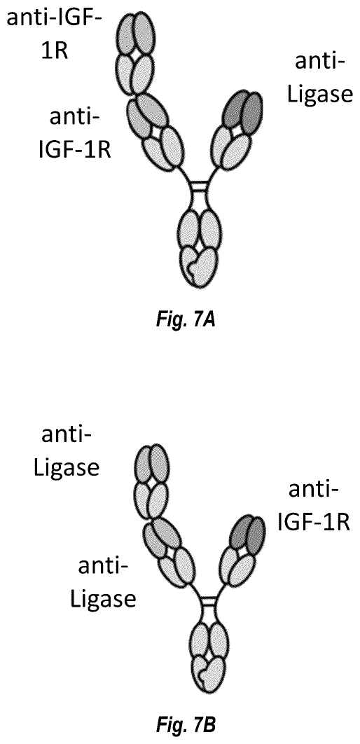

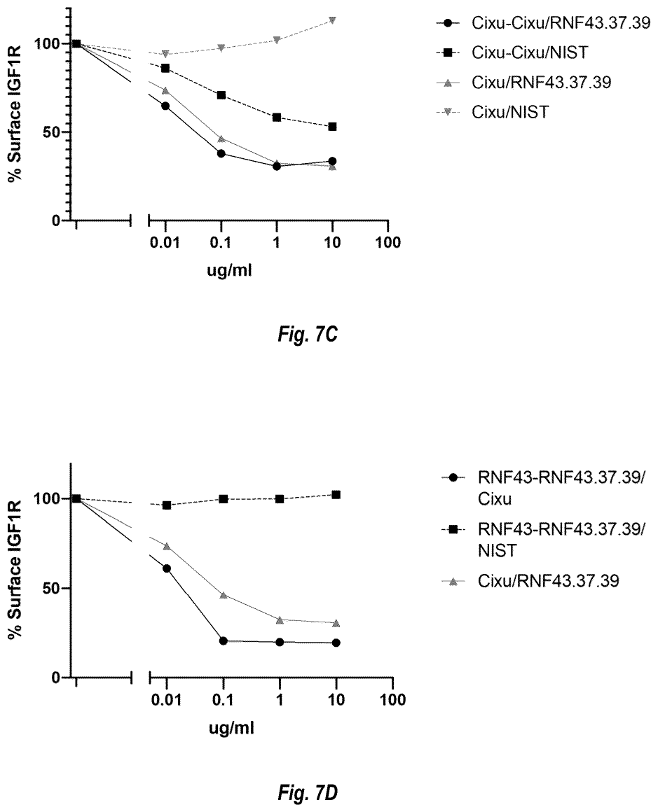

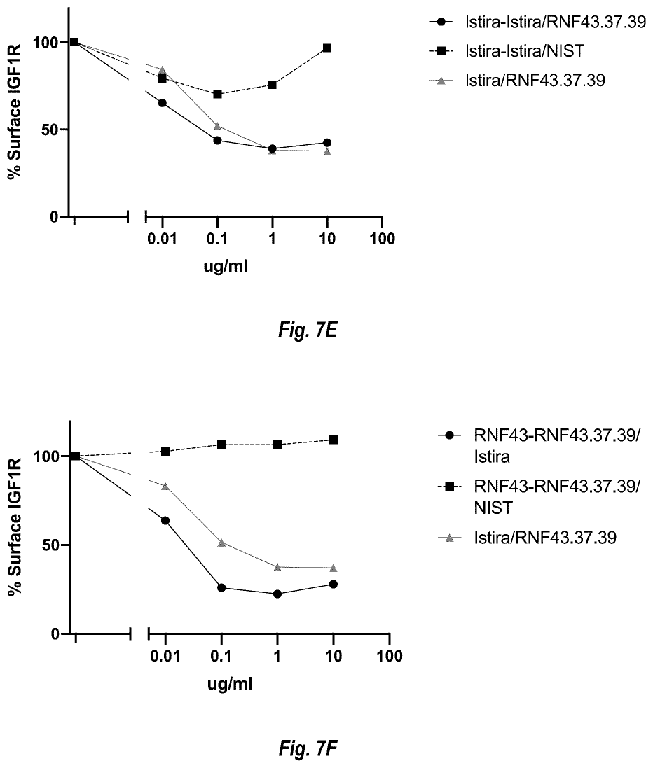

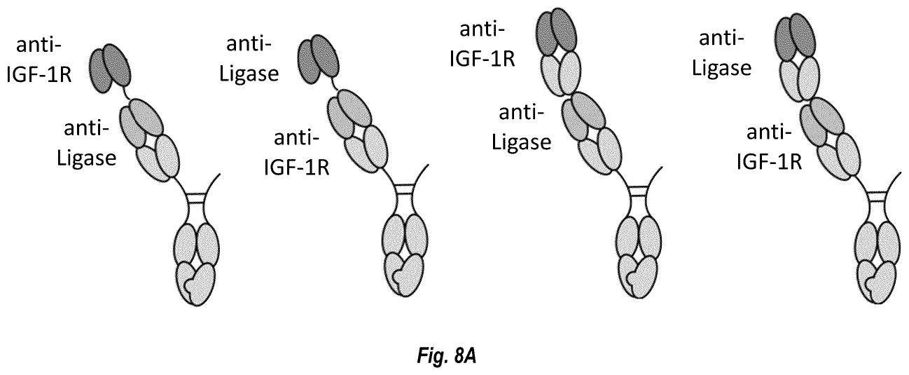

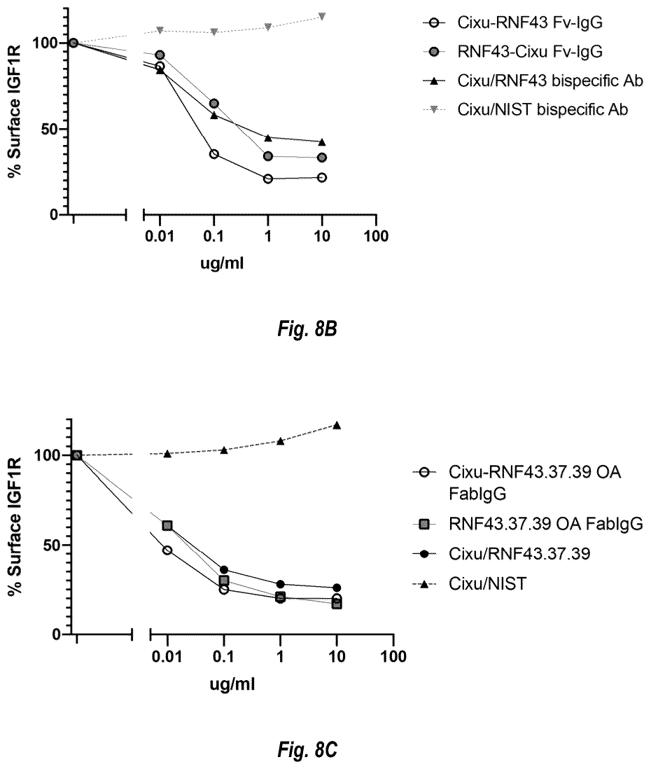

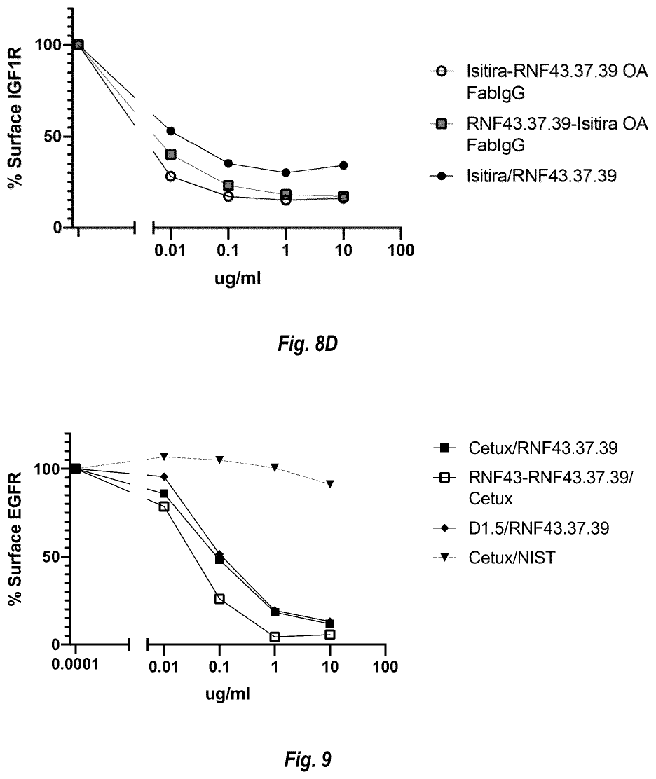

In some aspects, a multispecific binding protein herein may have one of the following formats, or another format tested in the Examples section or depicted in the figures herein:
a) a 2+1 FabIgG comprising an arm with two Fab regions binding to a transmembrane E3 ubiquitin ligase and an arm with one Fab region binding to a cell surface protein; (see, e.g., FIGS. 7B and 15A)
b) a 2+1 FabIgG comprising an arm with two Fab regions binding to a cell surface protein and an arm with one Fab region binding to a transmembrane E3 ubiquitin ligase; (see, e.g., FIGS. 7A and 15A)c) a one armed FvIgG comprising an Fv region binding to a cell surface protein and a Fab region binding to a transmembrane E3 ubiquitin ligase; (see, e.g., FIG. 8A, far left, and 15B);
d) a one armed FvIgG comprising an Fv binding to a transmembrane E3 ubiquitin ligase and a Fab binding to a cell surface protein (see, e.g., FIG. 8A, second from left, and 15B);
e) a one armed FabIgG comprising a first Fab region binding to a cell surface protein and a second Fab region binding to a transmembrane E3 ubiquitin ligase; (see, e.g., FIG. 8A, third from left, and 15C);
f) a one armed FabIgG comprising a first Fab region binding to a transmembrane E3 ubiquitin ligase and a second Fab region binding to a cell surface protein (see, e.g., FIG. 8A, far right, and 15C);
g) a 1+1 FabIgG comprising a first Fab region binding to a transmembrane E3 ubiquitin ligase and a second Fab region binding to a cell surface protein; and
h) a 1+1 FvIgG comprising a first Fv region binding to a transmembrane E3 ubiquitin ligase and a second Fv region binding to a cell surface protein. In any of the above, the protein may comprise at least one knob-into-hole modification in the Fc region, for example, which in some embodiments may reduce mis-pairing of the two halves of the IgG molecule.

In certain aspects, a multispecific binding protein such as a multispecific antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### B. Exemplary Anti-RNF43 and Anti-ZNRF3 Antibodies Useful in Constructing Multispecific Binding Molecules

In one aspect, the disclosure also provides antibodies that specifically bind to RNF43 and/or ZNRF3 that may be useful in constructing multispecific binding proteins according to the disclosure. These include the murine, rat, or rabbit antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, and ZNRF3-332, described in the Examples below, or humanized versions of those antibodies.

In some embodiments, a multispecific binding protein herein comprises a heavy chain variable domain (VH) comprising (a) a heavy chain complementarity determining region 1 (CDR-H1), CDR-H2, and/or CDR-H3 of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332. In some embodiments, the multispecific binding protein comprises a heavy chain variable domain (VH) comprising (a) a heavy chain complementarity determining region 1 (CDR-H1), CDR-H2, and CDR-H3 of any one of the above antibodies.

In some embodiments, the multispecific binding protein comprises a light chain variable domain (VL) comprising (a) a light chain complementarity determining region 1 (CDR-L1), CDR-L2, and/or CDR-L3 of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332. In some embodiments, it comprises a light chain variable domain (VL) comprising (a) a light chain complementarity determining region 1 (CDR-L1), CDR-L2, and CDR-L3 of any one of those antibodies. In some embodiments, the multispecific binding protein comprises all of the CDR-H1, CDR-H2, CDR-H3, CDR-L1, CDR-L2, and CDR-L3 of one of the above anti-RNF43 or anti-ZNRF3 antibodies.

In some cases, the multispecific binding protein comprises a heavy chain variable region (VH) comprising an amino acid sequence at least 85%, 86%, 87%, 88%, 89%, 90%, 91%, 92%, 93%, 94%, 95%, 96%, 97%, 98% or 99% identical to the VH of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332.

In some cases, the multispecific binding protein comprises a light chain variable region (VL) comprising an amino acid sequence at least 85%, 86%, 87%, 88%, 89%, 90%, 91%, 92%, 93%, 94%, 95%, 96%, 97%, 98% or 99% identical to the VH of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332.

In some cases, the multispecific binding protein comprises a VH comprising an amino acid sequence at least 85%, 86%, 87%, 88%, 89%, 90%, 91%, 92%, 93%, 94%, 95%, 96%, 97%, 98% or 99% identical to the VH of any one of above antibodies and also a VL comprising an amino acid sequence at least 85%, 86%, 87%, 88%, 89%, 90%, 91%, 92%, 93%, 94%, 95%, 96%, 97%, 98% or 99% identical to the VL of any one of above antibodies.

In some cases, the multispecific binding protein comprises a heavy chain variable region (VH) comprising an amino acid sequence of the VH of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332. In some cases, the multispecific binding protein comprises a light chain variable region (VL) comprising an amino acid sequence of the VL of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332. In some cases, the protein comprises both the VH and the VL of one of the above antibodies. The sequence table below provides DNA and protein sequences of the VH and VL of the above antibodies, from SEQ ID NO: 33 to SEQ ID NO: 444.

In further embodiments, a multispecific binding protein may be constructed using amino acid sequences provided in one or more of SEQ ID Nos: 1-32 in the sequence table below.

In some embodiments, the multispecific binding protein comprising the above CDRs or VH/VLs has a binding affinity for ZNRF3 or RNF43 of less than 50 nM, or less than 10 nM, or less than 1 nM, or less than 0.5 nM, or less than 0.05 nM, or between 50 nM and 10 nM, or between 10 nM and 1 nM, or between 1 nM and 0.5 nM, or between 0.5 nM and 0.05 nM, or between 0.05 nM and 0.01 nM. In some cases, affinity is measured using a BIACORE® surface plasmon resonance assay, for example, as described in the Examples below.

In some embodiments, the multispecific binding protein binds to both ZNRF3 and RNF43. For example, in some embodiments, the multispecific binding protein is a multispecific antibody that binds to both ZNFR3 and RNF43. In some embodiments, it is a trispecific antibody binding to those ligases and to a cell surface protein targeted for degradation. In some embodiments, it may have a format as shown in FIG. 15A-15G, FIG. 7A-B, or FIG. 8A.

In some embodiments, a multispecific binding protein comprising the above CDRs or VH/VLs is a rat anti-human or rabbit anti-human antibody or murine anti-human antibody. In other embodiments, it is humanized or chimeric. In some embodiments, the multispecific binding protein comprises a wild-type human Fc region, such as from a human IgG1, IgG2, IgG3, or IgG4. In other embodiments, the protein comprises a human IgG1 Fc with one or more amino acid substitutions, such as a LALAPG mutation or a substitution at N297, e.g., N297G or N297Q, for example, to render the Fc effectorless. In some embodiments, the multispecific binding protein does not have effector function. In other embodiments, the multispecific binding protein has effector function. These and other optional constant regions or Fc regions are also discussed further below.

### C. Conjugates Comprising Multispecific Binding Proteins

In other embodiments, a multispecific binding protein herein may further be conjugated (chemically bonded) to one or more other molecules, such as a label or a therapeutic agent. A variety of radioactive isotope labels are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron. Exemplary therapeutic agents include cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, a multispecific binding protein is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016). Other potential conjugated therapeutic agents include enzymatically active toxins or fragments thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

Conjugates of a protein and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### D. Antibody Variants

In certain aspects, amino acid sequence variants of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332 provided herein are contemplated. For example, it may be desirable to alter the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.
a) Substitution, Insertion, and Deletion Variants

In certain aspects, antibody variants having one or more amino acid substitutions are provided. Sites of interest for substitutional mutagenesis include the CDRs and framework regions of the variable regions. Conservative substitutions are shown in Table A under the heading of “preferred substitutions”. More substantial changes are provided in Table A under the heading of “exemplary substitutions”, and as further described below in reference to amino acid side chain classes. Amino acid substitutions may be introduced into an antibody of interest, e.g., in the variable and/or constant regions, and the products screened for a desired activity, e.g., retained/improved antigen binding, decreased immunogenicity, or improved ADCC or CDC.

TABLE A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

b) Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

c) Fc Region Variants

In certain aspects, one or more amino acid modifications may be introduced into the Fc region of an antibody provided herein, thereby generating an Fc region variant. The Fc region variant may comprise a human Fc region sequence (e.g., a human IgG₁, IgG₂, IgG₃ or IgG₄ Fc region) comprising an amino acid modification (e.g., a substitution) at one or more amino acid positions.

In some aspects, an antibody herein has effector function. In other aspects, an antibody herein lacks effector function. In certain aspects, the invention contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG₁ Fc region. In one aspect, the substitutions are L234A, L235A and P329G (LALAPG) in an Fc region derived from a human IgG₁ Fc region. (See, e.g., WO 2012/130831). In another aspect, the substitutions are L234A, L235A and D265A (LALA-DA) in an Fc region derived from a human IgG₁ Fc region.

In some aspects, the antibodies may have a modification at position N297 to reduce or eliminate ADCC activity, such as N297G or N297Q. In some such cases, the antibody lacks effector function.

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues 1253, H310, H433, N434, and H435 (EU index numbering) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG₁ Fc-region. See, e.g., Grevys, A., et al., J. Immunol. 194 (2015) 5497-5508

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG₁ Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG₁ Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

d) Cysteine Engineered Antibody Variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. No. 7,521,541, 830,930, 7,855,275, 9,000,130, or WO 2016040856.

e) Antibody Derivatives

In certain aspects, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

E. Recombinant Methods and Compositions

Proteins herein may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can be comprised in one or more nucleic acid molecules or expression vectors. Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. Examples of these bispecific antibodies are CrossMabs (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73) according to EU index numbering.

In one aspect, isolated nucleic acids encoding an antibody or other component of a multispecific binding protein as used in the methods as reported herein are provided.

In one aspect, a method of making a multispecific binding protein is provided, wherein the method comprises culturing a host cell comprising nucleic acid(s) encoding the antibody or components of the protein, as provided above, under conditions suitable for expression of the antibody, and optionally recovering the antibody or other protein components from the host cell (or host cell culture medium).

For recombinant production of an antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of protein-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for protein-encoding vectors, including fungi and yeast strains whose glycosylation pathways have been “humanized”, resulting in the production of an antibody with a partially or fully human glycosylation pattern. See Gerngross, T. U., Nat.

Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell).

### F. Pharmaceutical Compositions

In a further aspect, provided are pharmaceutical compositions comprising any of the multispecific binding proteins provided herein, e.g., for use in any of the below therapeutic methods. In one aspect, a pharmaceutical composition comprises any of the proteins provided herein and a pharmaceutically acceptable carrier. In another aspect, a pharmaceutical composition comprises any of the proteins provided herein and at least one additional therapeutic agent, e.g., as described below.

Pharmaceutical compositions of proteins as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized compositions or aqueous solutions. Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody compositions are described in U.S. Pat. No. 6,267,958. Aqueous antibody compositions include those described in U.S. Pat. No. 6,171,586 and WO 2006/044908, the latter compositions including a histidine-acetate buffer.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Pharmaceutical compositions for sustained-release may be prepared. Suitable examples of sustained-release preparations include semipermeable matrices of solid hydrophobic polymers containing the antibody, which matrices are in the form of shaped articles, e.g., films, or microcapsules.

The pharmaceutical compositions to be used for in vivo administration are generally sterile. Sterility may be readily accomplished, e.g., by filtration through sterile filtration membranes.

### G. Therapeutic Methods and Routes of Administration

Any of the multispecific binding proteins provided herein may be used in therapeutic methods. For example, they may be useful in any therapeutic method in which it is beneficial to reduce the level of a particular cell surface protein in a subject by targeting it for lysosomal degradation, and in which it is beneficial to do so in cells that readily express a transmembrane E3 ubiquitin ligase.

In one aspect, an multispecific binding protein for use as a medicament is provided. In further aspects, an multispecific binding protein for use in treating a disease such as cancer is provided. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer, an autoimmune condition, an inflammatory condition, a neurodegenerative condition, or an infectious disease.

In certain aspects, an multispecific binding protein for use in a method of treatment is provided. In certain aspects, the invention provides an multispecific binding protein for use in a method of treating an individual having a disease such as cancer, an autoimmune condition, an inflammatory condition, a neurodegenerative condition, or an infectious disease, comprising administering to the individual an effective amount of the multispecific binding protein. In one such aspect, the method further comprises administering to the individual an effective amount of at least one additional therapeutic agent (e.g., one, two, three, four, five, or six additional therapeutic agents), e.g., as described below.

In further aspects, the invention provides an multispecific binding protein for use in reducing the level of a cell surface protein in a subject that is targeted by the multispecific binding protein. In some cases, the subject may have a disease such as cancer, an autoimmune condition, an inflammatory condition, a neurodegenerative condition, or an infectious disease.

In a further aspect, the invention provides for the use of an multispecific binding protein in the manufacture or preparation of a medicament. In one aspect, the medicament is for treatment of a disease such as cancer, an autoimmune condition, an inflammatory condition, a neurodegenerative condition, or an infectious disease. In a further aspect, the medicament is for use in a method of treating a disease such as cancer, an autoimmune condition, an inflammatory condition, a neurodegenerative condition, or an infectious disease, comprising administering to an individual having cancer an effective amount of the medicament. In one such aspect, the method further comprises administering to the individual an effective amount of at least one additional therapeutic agent, e.g., as described below. In a further aspect, the medicament is for in reducing the level of a cell surface protein in a subject that is targeted by the multispecific binding protein. In some cases, the subject may have a disease such as cancer, an autoimmune condition, an inflammatory condition, a neurodegenerative condition, or an infectious disease.

In a further aspect, the invention provides a method for treating a disease such as cancer. In one aspect, the method comprises administering to an individual having such cancer, an autoimmune condition, an inflammatory condition, a neurodegenerative condition, or an infectious disease, an effective amount of an multispecific binding protein. In one such aspect, the method further comprises administering to the individual an effective amount of at least one additional therapeutic agent, as described below.

In a further aspect, the invention provides a method for in reducing the level of a cell surface protein in a subject that is targeted by the multispecific binding protein. In some cases, the subject may have a disease such as cancer, an autoimmune condition, an inflammatory condition, a neurodegenerative condition, or an infectious disease. For example, in some aspects, multispecific binding proteins herein may be used to reduce levels of a cell surface protein on particular cell types by targeting an E3 ubiquitin ligase that is primarily expressed on those cell types. For example, the transmembrane E3 ubiquitin ligases RNF130, RNF149, and RNF167 are expressed on hematopoietic cells and multispecific binding proteins targeting those ligases could be used in some embodiments to reduce levels of cell surface proteins on those cells. Additionally, the transmembrane E3 ubiquitin ligases RNF133 and RNF148 are expressed on testicular cells and multispecific binding proteins targeting those ligases could be used in some embodiments to reduce levels of cell surface proteins on those cells.

In a further aspect, the invention provides pharmaceutical compositions comprising any of the multispecific binding proteins provided herein, e.g., for use in any of the above therapeutic methods. In one aspect, a pharmaceutical composition comprises any of the multispecific binding proteins provided herein and a pharmaceutically acceptable carrier. In another aspect, a pharmaceutical composition comprises any of the multispecific binding proteins provided herein and at least one additional therapeutic agent, e.g., as described below.

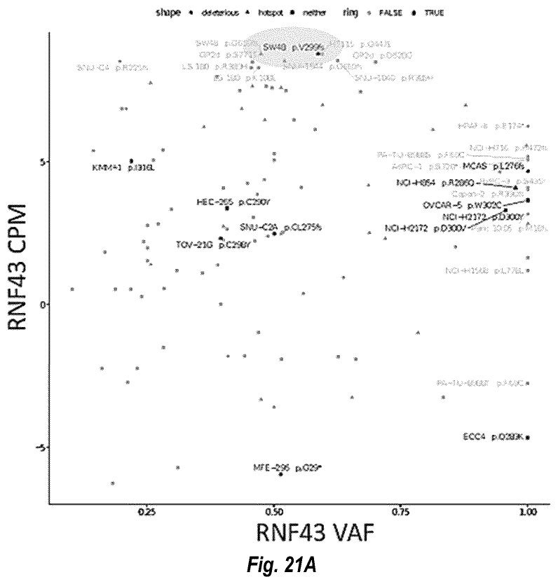

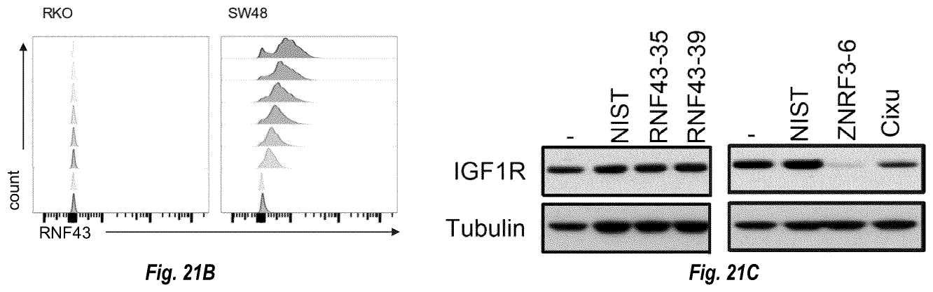

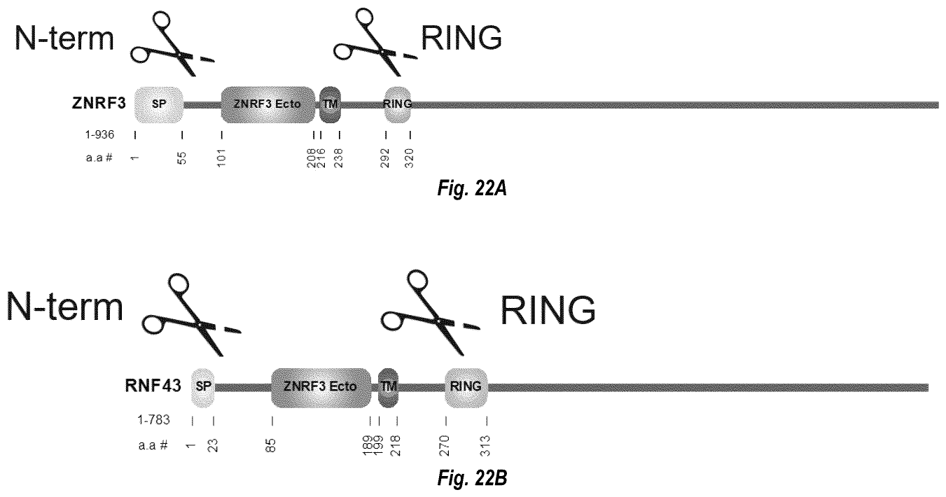

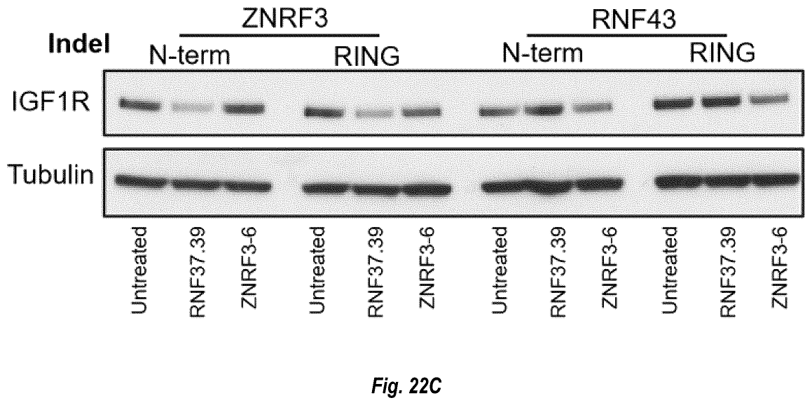

In other aspects of the uses and methods of treatment herein, a subject may have a mutation in the RNF43 or ZNRF3 protein. For example, in certain cancers, such as colorectal cancer and endometrial cancer, among others, the RNF43 protein has been found to be mutated. See, e.g., Y. J. van Herwaarden et al., Histopathology 78: 749-758 (2021). In such cases, a multispecific binding protein comprising an anti-RNF43 component may be less effective in reducing the level of a targeted cell surface protein. However, a multispecific binding protein comprising an anti-ZNRF3 component may readily lead to reduction in the level of the targeted cell surface protein. (See, e.g., Example 29 below and FIGS. 21-22.) Thus, in some embodiments, where a subject has been determined previously to have an RNF43 or ZNRF3 mutation, for example, where a subject has a cancer comprising cancer cells having a mutated RNF43 or ZNRF3, the subject may be administered a multispecific binding protein that does not bind to or does not activate the RNF43 or ZNFR3 protein. Thus, for example, if a subject has an RNF43 mutation, a multispecific binding protein provided to that subject may comprise an anti-ZNRF3 component, and vice versa. Accordingly, in some aspects, treatment methods and uses may also comprise, prior to administering the multispecific binding protein, determining whether the subject has a mutation in RNF43 or ZNRF3, wherein, (a) if the subject has a mutation in RNF43, the multispecific binding protein does not bind to or does not activate RNF43, and (b) if the subject has a mutation in ZNRF3, the multispecific binding protein does not bind to or does not activate ZNRF3. Such mutations may be identified, for example, at the protein level by techniques such as immunohistochemistry (IHC), or they may be identified at the nucleic acid level by techniques such as reverse-transcription polymerase chain reaction (RT-PCR), whole genome sequencing, exome sequencing, or in situ hybridization or the like. Because such mutations may be present in certain cancers, in some such aspects, the subject may be a cancer subject.

Proteins herein can be administered alone or used in a combination therapy. For instance, the combination therapy includes administering a multispecific binding protein and administering at least one additional therapeutic agent (e.g. one, two, three, four, five, or six additional therapeutic agents). Such combination therapies noted above encompass combined administration (where two or more therapeutic agents are included in the same or separate pharmaceutical compositions), and separate administration, in which case, administration of the antibody of the invention can occur prior to, simultaneously, and/or following, administration of the additional therapeutic agent or agents.

A multispecific binding protein of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic.

### H. Kits and Articles of Manufacture and Other Methods of Use

In another aspect of the disclosure, multispecific binding molecules herein may be used in vitro, i.e. in the laboratory to modify the level of a cell surface protein of a cell culture sample or tissue sample. For instance, there are numerous instances in which it may be beneficial to assay the behaviour of a cell or tissue sample in which the level of a particular cell surface signaling molecule is artificially reduced. Employing such a multispecific binding molecule may provide a relatively simply way to modulate the level of such a protein.

Thus, the present disclosure also encompasses methods of reducing the level of a cell surface protein in a cell or tissue sample in vitro, comprising incubating the sample with the multispecific binding protein. The present disclosure also encompasses kits for this purpose. Kits may comprise the multispecific binding protein, optionally also with instructions for use, appropriate buffers, and/or labeling molecules. Kits may also comprise nucleic acids, vectors, or host cells that comprise polynucleotides encoding the multispecific binding protein, so that the protein may, for example, be produced for use in such methods.

### III. Examples

The following are examples of methods and compositions of the invention. It is understood that various other embodiments may be practiced, given the general description provided above.

### Example 1: Development and Characterization of Rat Anti-Human Cell Surface Ligase Rat B Cell Antibodies or Rabbit B Cell Antibodies

Rats and rabbits were chosen for the generation of cell surface ligase antibodies because of certain advantages provided over alternative methods, e.g., screening of phage libraries. For example, antibodies obtained from animals may have greater specificity and better pharmacokinetic properties than phage-derived antibodies leading to better therapeutic candidates than phage-derived antibodies. Sprague Dawley rats (Charles River, Hollister, CA) were immunized with a priming dose of 100 μg human (RNF43 or ZNRF3) protein solubilized in detergent mixed with MPL+TDM adjuvant (Sigma-Aldrich, St. Louis, MO), CFA (Sigma-Aldrich, St. Louis, MO) or mixed with a combination of TLR agonists: 50 ug MPL (Sigma-Aldrich), 20 ug R848 (Invivogen, San Diego, CA), 10 ug PolyI:C (Invivogen), and 10 ug CpG (Invivogen) divided among multiple sites. New Zealand white rabbits were also immunized with a mixture of the same proteins solubilized in detergent mixed with CFA (Sigma-Aldrich, St. Louis, MO). For additional protein boosts, the rats and rabbits received half amount of the priming dose protein diluted in PBS. Rats and rabbits were dosed every two weeks. Polyclonal antisera from these rats and rabbits were purified and tested by ELISA for binding to human RNF43 and human ZNRF3. For the rats, multiple lymph nodes were harvested three days after the last immunization that showed detectable FACS reactivity against human RNF43 or human ZNRF3. IgM-negative B-cells from these rats were purified from whole lymphocytes using magnetic separation (Miltenyi Biotec, San Diego, CA) and stained with anti-rat IgM antibody (Jackson ImmunoResearch, West Grove, PA), anti-rat CD45RA (Biolegend, San Diego, CA), anti-rat CD8a (Biolegend, San Diego, CA), and labeled human RNF43-Alex 633 or human ZNRF3-Alex633 by Lightning-Link Alex 633 Antibody Labeling kit (Novus, Centennial, CO). Rat B-cells showing minimal rat IgM expression while binding to the human RNF43 or human ZNRF3 protein were sorted and deposited into 96-well plates containing a culture medium containing feeder cells and supplemented with cytokines using a FACSAriaIII sorter (BD, Franklin Lakes, NJ). For the rabbits, the blood was collected three days after the last immunization that showed detectable FACS reactivity against human RNF43 and human ZNRF3. IgG-positive B cells from these rabbits were purified from the whole blood using magnetic separation (Miltenyi Biotec, San Diego, CA) and stained with anti-rabbit IgG antibody (Southern Biotech, Birmingham, AL) and labeled human RNF43-Alex 633 or human ZNRF3-Alex633 by Lightning-Link Alex 633 Antibody Labeling kit (Novus, Centennial, CO). Rabbit B-cells showing maximum rabbit IgG expression while binding to the human RNF43 or human ZNRF3 protein were sorted and deposited into 96-well plates containing a culture medium containing feeder cells and supplemented with cytokines using a FACSAriaIII sorter (BD, Franklin Lakes, NJ). Supernatants were screened by ELISA against human RNF43 or human ZNRF3 seven days after sorting. Supernatants demonstrating human RNF43 or human ZNRF3 binding were tested by FACS for binding to human RNF43 or human ZNRF3 expressed on the surface of gD-hRNF43 or binding to human ZNRF3 expressed on the surface of gD-hZNR3. RNA was extracted from B-cells that showed RNF43 or ZNRF3 FACS binding for molecular cloning and recombinant expression. Recombinant antibodies were tested by FACS for binding to human RNF43 expressed on the surface of gD-hRNF43 or binding to human ZNRF3 expressed on the surface of gD-hZNR3.

### Example 2: Recombinant Antibody Generation

DNA encoding antibody heavy and light chain variable domains was generated by gene synthesis. The synthesized genes fragments were inserted into mammalian expression vectors containing the corresponding heavy or light constant domains. Species and isotypes included human IgG1 and murine IgG2a. Some variable domain sequences were edited to remove apparent unpaired cysteine residues and NX[S/T] N-glycosylation motifs. Recombinant antibodies were produced by transient transfection of Expi293 cells with mammalian expression vectors encoding the antibody heavy chain and light chain. Heavy chain and light chain were encoded on separate vectors, and were transfected using a 1:1 ratio of heavy chain expression vector to light chain expression vector. Antibodies were purified from the cell culture supernatant by affinity chromatography. In some cases, antibodies underwent an additional purification step based on SEC.

Bispecific antibodies were generated using knob-into-hole technology (Ridgway et al., Prot Eng. 1996) using human IgG1 or murine IgG2a backbones typically including mutations to reduce effector function (e.g. L234A, L235A, P329G, and/or N297G). Abs containing either knobs or holes were expressed and purified prior to assembly into bispecific format essentially as previously described (Williams et al., Biotechnol Prog., 2015). In some cases, mutations were introduced to enable bispecific expression in a single cell including appropriate light-chain pairing (Dillon et al., mAbs, 2017).

### Example 3: Affinity and Epitope Determination for Recombinant Antibodies

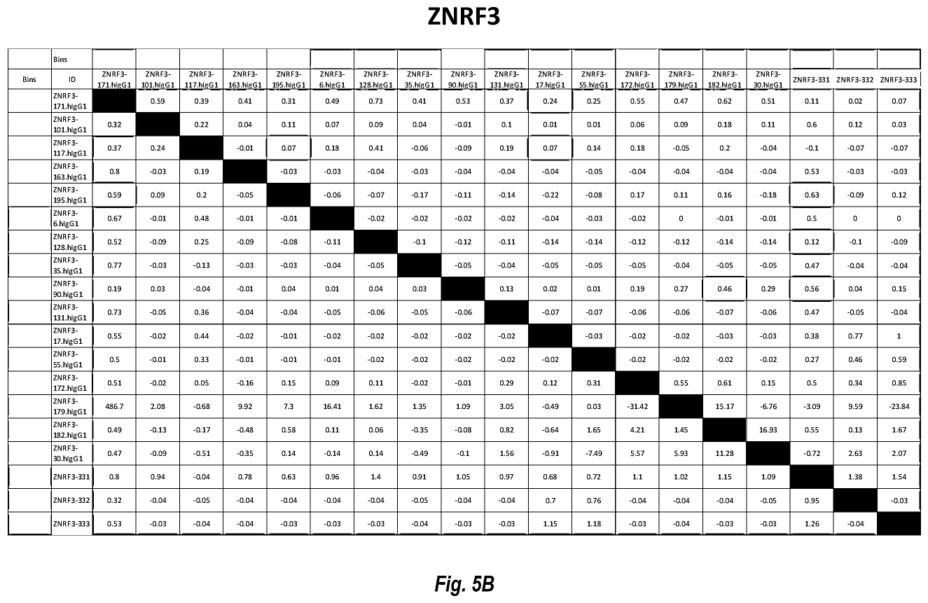

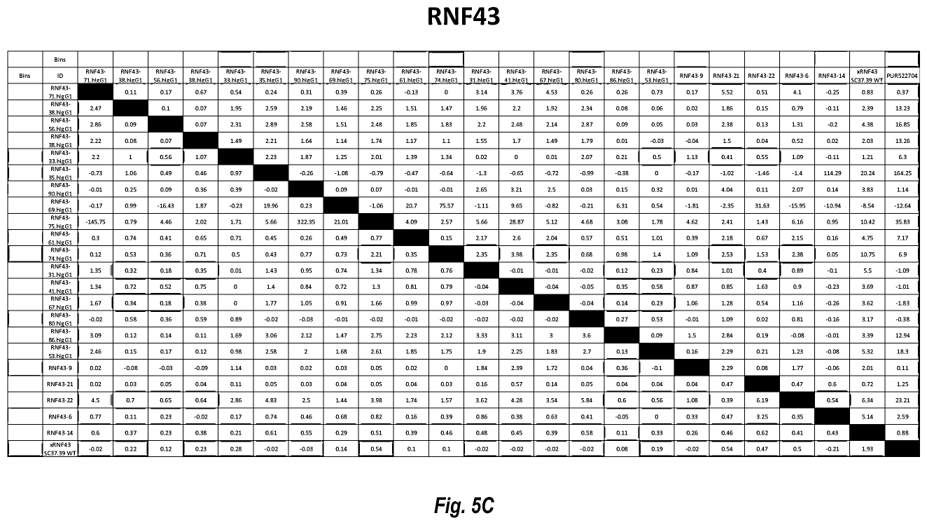

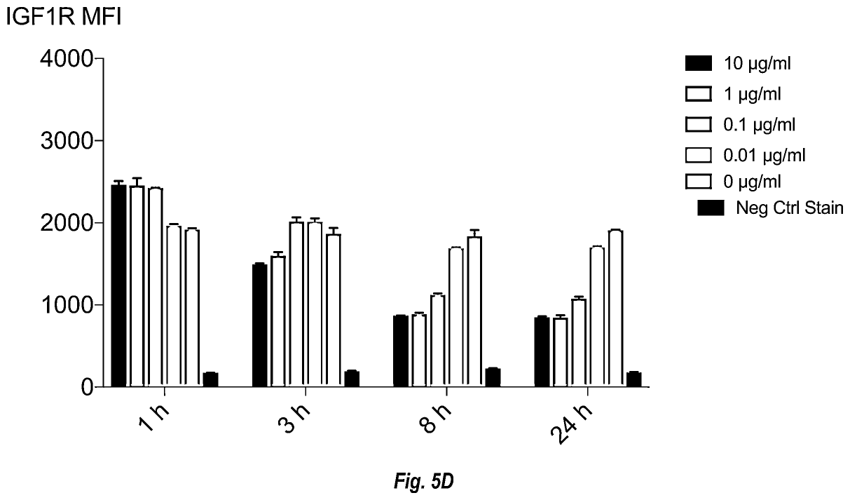

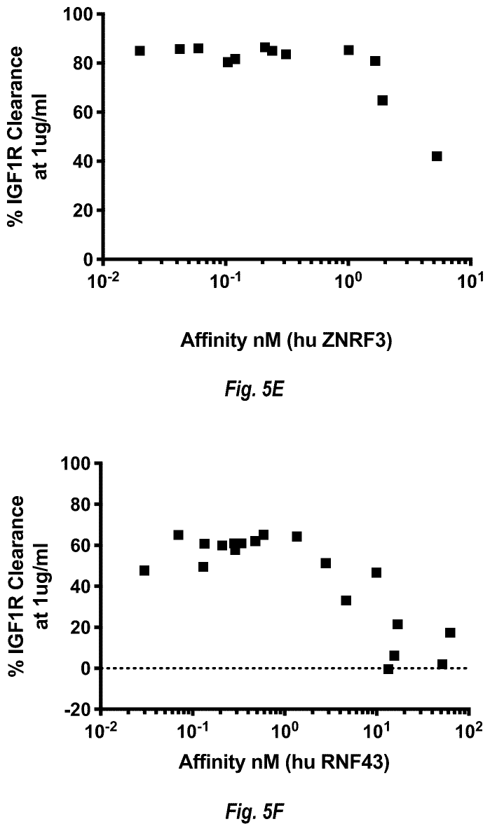

Antibodies were screened for binding to recombinant human RNF43 and ZNRF3 ECDs using a Biacore® 8 k instrument (GE Life Sciences). Briefly, antibodies diluted to 1 μg/ml in 1×HBSP buffer (Cytiva, BR100368) were captured on a Sensor Chip Protein A (GE Life Sciences) using a flow rate of 10 μl/minute and a contact time of 60 seconds. Binding of recombinant human RNF43 and ZNRF3 extracellular domain to the captured antibodies was analyzed at 25° C. using a single cycle kinetics method with a flow rate of 30 μl/minute, a contact time of 180 seconds and a dissociation time of 600 seconds. The concentration of recombinant human RNF43 and ZNRF3 extracellular domain in the single cycle kinetics were 0, 0.8, 4, 20, and 100 nM. Between cycles, the chip was regenerated using 10 mM Glycine HCl pH 1.5 injected for 30 seconds at 30 μl/minute. Data were evaluated using Biacore 8K Evaluation software (GE Life Sciences). Kinetic constants were obtained using a 1:1 binding model with the parameter RI set to zero. Multiple antibodies targeting RNF43 and ZNRF3 were shown to have sub-nanomolar affinities as shown in Tables 1-4. Cell surface ligase antibodies with sub-nanomolar affinities may provide advantages in certain instances, for example, in the degradation efficiency which is correlated with the durability of the ternary complex as shown in FIG. 5D.

TABLE 1
Binding of rat clones to huRNF43

| Clone | Affinity (nM) |
|---|---|
| RNF43-71 | 0.03 |
| RNF43-25 | |
| RNF43-38 | 4.93 |
| RNF43-56 | 1.84 |
| RNF43-38 | 8.66 |
| RNF43-33 | 62.97 |
| RNF43-35 | 0.07 |
| RNF43-90 | 0.29 |
| RNF43-69 | 16.8 |
| RNF43-75 | 4.65 |
| RNF43-61 | 0.43 |
| RNF43-74 | 0.35 |
| RNF43-31 | 0.13 |
| RNF43-41 | 0.37 |
| RNF43-67 | 0.59 |
| RNF43-80 | 0.48 |
| RNF43-86 | 17.27 |
| RNF43-53 | 3.3 |
| RNF43-186 | |
| RNF43-108 | |
| RNF43-181 | 8.59E−02 |
| RNF43-196 | 0.114 |
| RNF43-216 | 0.291 |
| RNF43-130 | 0.134 |
| RNF43-136 | 1.91 |
| RNF43-177 | 6.47 |
| RNF43-116 | 1.42 |
| RNF43-104 | 9.12 |
| RNF43-145 | 0.504 |
| RNF43-156 | 0.209 |
| RNF43-224 | 0.584 |
| RNF43-168 | 1.36 |
| RNF43-176 | 5.71 |
| RNF43-200 | 0.539 |
| RNF43-206 | 0.58 |
| RNF43-220 | 1.57 |
| RNF43-128 | N.B. |
| RNF43-126 | 1.75 |
| RNF43-217 | 1.49 |
| RNF43-170 | 76 |
| RNF43-179 | 31.7 |
| RNF43-221 | 51.6 |
| RNF43-180 | 2.81 |
| RNF43-129 | 1.17 |
| RNF43-107 | 0.341 |
| RNF43-117 | 19.8 |
| RNF43-152 | 17.8 |
| RNF43-201 | 9.9 |
| RNF43-106 | 15.6 |
| RNF43-123 | 13.4 |
| RNF43-210 | 13.4 |

TABLE 2
Binding of rat clones to huZNRF3

| Clone | Affinity (nM) |
|---|---|
| ZNRF3-171 | 1.01 |
| ZNRF3-101 | 0.07 |
| ZNRF3-117 | 0.2 |
| ZNRF3-170 | |
| ZNRF3-163 | 0.61 |
| ZNRF3-195 | 0.27 |
| ZNRF3-6 | 0.12 |
| ZNRF3-128 | 3.16 |
| ZNRF3-35 | |
| ZNRF3-90 | 0.27 |
| ZNRF3-131 | 2.39 |
| ZNRF3-17 | 0.04 |
| ZNRF3-55 | 0.02 |
| ZNRF3-172 | 0.21 |
| ZNRF3-179 | 0.11 |
| ZNRF3-182 | 0.06 |
| ZNRF3-30 | 0.12 |
| ZNRF3-305 | 7.18 |
| ZNRF3-312 | 0.611 |
| ZNRF3-279 | 1.66 |
| ZNRF3-275 | 0.0565 |
| ZNRF3-314 | 0.104 |
| ZNRF3-301 | 0.0974 |
| ZNRF3-269 | 0.312 |
| ZNRF3-219 | 1.9 |
| ZNRF3-265 | 0.0286 |
| ZNRF3-253 | 0.483 |
| ZNRF3-254 | 0.104 |
| ZNRF3-300 | 0.239 |
| ZNRF3-222 | N.B. |
| ZNRF3-287 | 0.191 |
| ZNRF3-322 | N.B. |
| ZNRF3-247 | 0.161 |
| ZNRF3-296 | 0.0371 |
| ZNRF3-223 | 0.0443 |
| ZNRF3-231 | 0.0783 |
| ZNRF3-244 | 6.85 |
| ZNRF3-270 | 0.0424 |
| ZNRF3-329 | 0.283 |
| ZNRF3-255 | 0.26 |
| ZNRF3-237 | 5.3 |

TABLE 3
Binding of rabbit clones to huRNF43

| Clone | Affinity (nM) |
|---|---|
| RNF43-13 | 99.6 |
| RNF43-19 | 34.2 |
| RNF43-9 | 1.8 |
| RNF43-2 | N.B. |
| RNF43-21 | 10.3 |
| RNF43-22 | 0.5 |
| RNF43-23 | 85 |
| RNF43-6 | 10.3 |
| RNF43-14 | 0.8 |
| RNF43-17 | 6.7 |
| RNF43-15 | 8 |
| RNF43-5 | 1.1 |
| RNF43-11 | 27.7 |
| RNF43-16 | 37.8 |
| RNF43-20 | N.B. |

TABLE 4
Binding of rabbit clones to huZNRF3

| Clone | Affinity (nM) |
|---|---|
| ZNRF3-331 | 0.2 |
| ZNRF3-332 | 1.6 |
| ZNRF3-333 | 8.7 |

### Example 4: HTP Epitope Binning

Antibodies generated by animal immunization were reformatted into hIgG1 backbones and binning was performed using the CFM2/MX96 SPR system (Wasatch Microfluidics, now Carterra), equipped with DA v6.19.3, IBIS SUIT, SprintX & Carterra Epitope Tool software. 10 μg/ml antibodies were immobilized on an SPR sensor prism CMD 200 M (Xantec Bioanalytics) by amine coupling using a 10 mM sodium acetate pH 4.5 immobilization buffer. Immobilization was performed with the CFM2 instrument and the sensor prism was then transferred to the IBIS MX96 instrument for SPR-based competition analysis. Immobilized antibodies were exposed first to 100 nM recombinant human RNF43 or ZNRF3 extracellular domain and then to 10 ug/ml antibody in solution, using an HBS-EP running buffer (10 mM HEPES, 150 mM NaCl, 0.05% Tween 20, pH 7.4, 1 mM EDTA). 10 mM glycine/HCl pH 1.7 were used as the regeneration buffer. Epitope bin results are shown in FIG. 5b and indicate several different RNF43 and ZNRF3 sandwiching profiles indicating binding to multiple different epitopes on the antigens.

### Example 5: Assessing Target Cell-Surface Clearance by Flow Cytometry

Antibodies were assessed for their ability to remove target antigens from the cell surface of multiple target cells. Cell lines included SW1417, HT29, and engineered HT-29 cell lines including ones engineered to express N-terminally tagged ligases. N-terminal tags included an epitope tag (gD) or a HiBit tag (Promega) for quantitation of tagged proteins through the Nano-glo HiBit Extracellular Detection System (Promega). Cell lines were pools or clonal and optionally included ligase expression on a doxycycline inducible promoter. Cells were plated into 96-well plates at 50,000-120,000 cells wells and grown in ATCC recommended media (+/−doxycycline) in the presence of various concentrations of antibody. After 24 hours (or the indicated period of time), media was removed, cells were washed with PBS (150 uL), and detached from the well surface by addition of accutase (Millipore) (100 uL) at 37° C. for 10 min. Accutase activity was quenched by addition of 100 uL of RPMI+10% heat inactivated Fetal bovine serum with penicillin, streptomyces, and glutamine, cells were centrifuged for 4 min @1200 rpm, and the supernatant was discarded. Cells were resuspended in 150 uL FACS buffer (PBS with 0.5% BSA and 0.05% sodium azide) for 10 min at 4° C., centrifuged and supernatant discarded. Cells were incubated with 40 uL of staining antibodies (2 ug/ml final concentration) for 45 min at 4° C., washed twice in 150 uL of FACS buffer and resuspended in 40 uL of FACS buffer for analysis. Staining antibodies include xIGF-1R mIgG1 APC (1H7, ebioscience catalog #17-8849-42) and xRNF43 37.17 APC (in-house generated reagent). Background/negative control staining antibodies include NISTMab hIgG1APC and xRagweed mIgG1 APC (both in-house generated reagents).

Flow cytometry analysis was performed on a BD FACSCelesta™ Flow Cytometer using the High Throughput Sampler (HTS) in high throughput mode. Each sample was first resuspended twice with 10 ul of resuspension volume. Then 10 ul of sample was aspirated from each well and flow through the cytometer at the flow rate of 180 ul/min. Between each samples the system was washed with 400 ul (wash volume) of buffer. The SSC, FSC, and APC signals were measured. Using Flowjo FACS analysis software, cells were gated for single cell based on the SSC and FSC profile. Mean Fluorescent Intensity (MFI) of APC signal as a measure of IGF-1R level on the cell surface.

### Example 6: Genetic Induced Dimerization of Ligases to Cell Surface Receptors

The effect of chemically induced dimerization of RNF43 or ZNRF3 with HER2 was assessed using the iDimerize system (Takara Bio). Briefly, HEK293T cells were reverse transfected with pBind plasmids to induce the constitutive co-expression of RNF43-DmrA-HA and HER2-DmrC-FLAG or ZNRF3-DmrA-HA and HER2-DmrC-FLAG. After 24 hours, cells were either left untreated or treated with 500 nM A/C heterodimerizer (TakaraBio; REF 635057) to induce ligase-target dimerization and cells were incubated for an additional 24 hours. Cells were then lysed in GST lysis buffer [25 mM Tris·HCl, pH 7.2, 150 mM NaCl, 5 mM MgCl2, 1% NP-40 and 5% glycerol, 1% Halt protease & phosphatase inhibitor cocktail]. Cleared lysates were subjected to an HA immunoprecipitation (IP) by incubating lysates with 25 μl Pierce HA epitope tag antibody agarose conjugate (2-2.2.14) (Thermo Scientific; REF 26182) for 3 hours at 4° C. For input samples, 20 μl were used from the prepared IP samples prior to incubation. Beads were washed following incubation and prepared for Western blot analysis.

### Example 7: Western Blot and Immunoprecipitation

HT29, HEK293T and SW1417 cells were plated in 6 well dish (Corning, cat #3516) at a density of 500,000 cells per well and let adhere overnight. Cells were treated with bispecific antibodies diluted at lug/mL in RPMI-1640 media supplemented with FBS, L-glutamine, and Pen/Strep for 40 hours. Where applicable, cells were pre-treated with doxycycline (500 ng/ml), Bortezomib (200 nM, Selleck, cat #S1013), E1 inhibitor (MLN7243, cat #S8341, 100 nM), or Bafilomycin A (Tocris —Cat. No. 1334, 50 nM). Following 24-48 hour incubation, cells were lysed in RIPA buffer supplemented with Phosphatase/Protease inhibitor (Thermo Fisher, Cat #78442) for 20 minutes on ice. Lysates were clarified and normalized, and 20 ug of each sample was run on 4-12% Bis-Tris gel (Invitrogen, Cat #WG1403) using BOLT MOPS SDS running buffer at 90V for approximately 2 hours. The gel was transferred to Nitrocellulose membrane using iBlot2 system using iBlot2 Nitrocellulose Regular stacks (Invitrogen, Cat #1B23001) and transferred using protocol PO (1 minute at 20V, 4 minutes at 23V, and 2 minutes at 25V). Blots were blocked with 5% milk in TBST and blocked for 1 hour at room temperature. Blots were transferred to 5% milk in TBST containing antibodies for IGF1R-beta (Cell signaling, #3027), Vinculin (Cell signaling, cat #13901), and incubated at 4 degrees overnight. Blots were washed 4 times for 5 minutes each with TBST before being incubated in 5% milk containing IRDye secondary antibody (Li-Cor, Cat #926-32211) for 1 hour at room temperature. Blots were washed 4 times for 5 minutes each and developed using the Li-Cor system.

### Example 8: Characterization of Cell Surface Ligases Driven by Wnt Signaling Activity

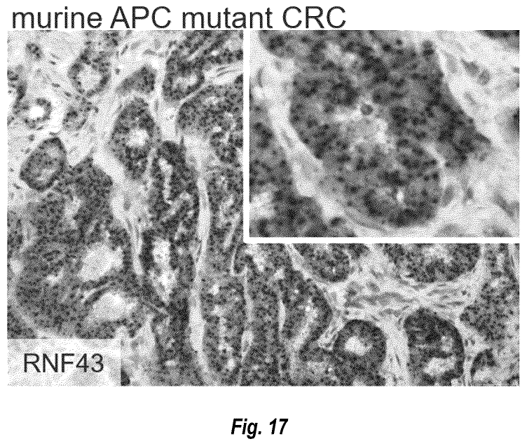

RNF43 and ZNRF3 are Wnt canonical targets that control the turnover of the Wnt ligands receptors FZD and LRP at the cell surface (FIG. 1A). This is achieved through ubiquitination and consequently cell surface clearance of FZDs/LRP by RNF43/ZNRF3 (FIG. 1A). To evaluate the impact of oncogenic Wnt signaling on RNF43 and ZNRF3 expression, we made use of publicly available gene expression profiles from various colorectal cancer patients data sets, where Wnt signaling is constitutively hyperactive. Both cell surface ligases displayed elevated expression in colorectal adenoma samples compared to healthy control normal mucosa (Galamb O., et al., Dis Markers, 2008. GSE4183; FIG. 1B). In addition, colorectal cancer gene expression analysis derived from the cancer genome atlas (TCGA) demonstrated elevated expression of RNF43 in CRC compared to other tumor types and normal colon (FIG. 1C). Importantly, in situ hybridization of Rnf43 in murine colon cancer models demonstrated broad and homogeneous expression of the ligase within the entire tumor mass (FIG. 17). The canonical role of RNF43 and ZNRF3 was validated using transfection of an inducible plasmid that conditionally expresses either ligases into HPAF-II cells upon addition of doxycycline (dox). Dox mediated over expression of WT RNF43 leads to cell surface clearance of FZD receptors in a ligase dependent manner (FIG. 1D). To probe the degradative potential of RNF43 and ZNRF3 beyond their natural substrates, namely FZDs and LRP5/6, we took advantage of the idimerize system. HEK293T were transfected with pBind plasmids to induce the constitutive co-expression of RNF43-DmrA-HA and HER2-DmrC-FLAG. 24 hr after addition of the AC heterodimerizer, an increase binding between RNF43 and HER2 was detected through immunoprecipitation of RNF43 followed by immunoblotting of HER2 (FIG. 1E left panel). This increase in ligase-target binding was function as it resulted in HER2 target degradation (FIG. 1E right panel).

### Example 9: Expression and Dimerization of Tagged RNF43 and ZNRF3 Ligases to HER2

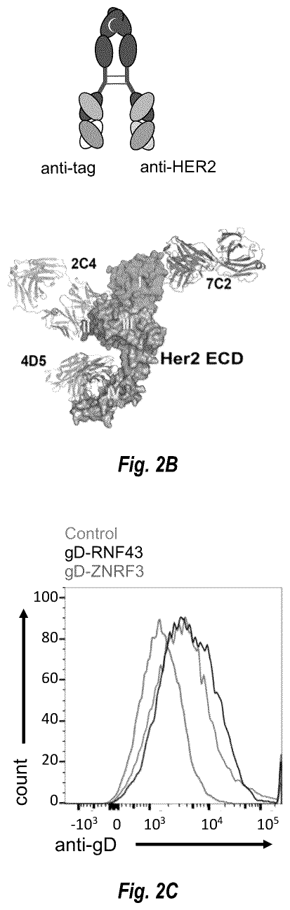

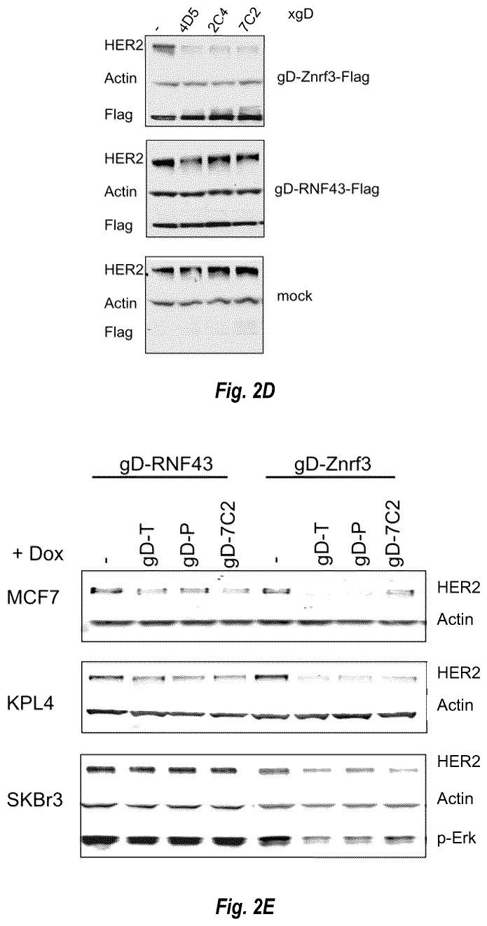

We further evaluated the ability of both cell surface ligases to degrade receptors when engaged with bispecific antibodies. N-terminally gD tagged ligases were generated, and dimerized to HER2 by using bispecific targeting three different epitopes of HER2 (4D5, 7C2, and 2C4) (FIG. 2A-B) and gD. Cell surface expression of both ligases was confirmed by flow cytometry (FIG. 2C) and gD based dimerization through either HER2 epitopes led to HER2 degradation, which was dependent on the ligase expression (FIG. 2D). We further validated this degradation potential in additional HER2 expressing breast cancer cell lines, including the HER2 amplified KPL4 cell line. Importantly, HER2 degradation also impacted downstream signaling as evidenced by a decrease in pERK activity (FIG. 2E).

### Example 10: Antibody Dependency of Cell Surface Clearance

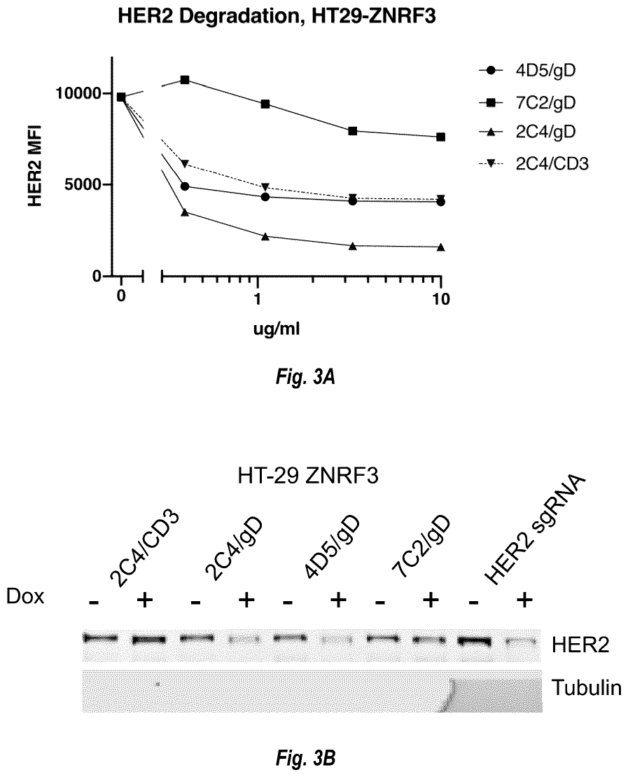

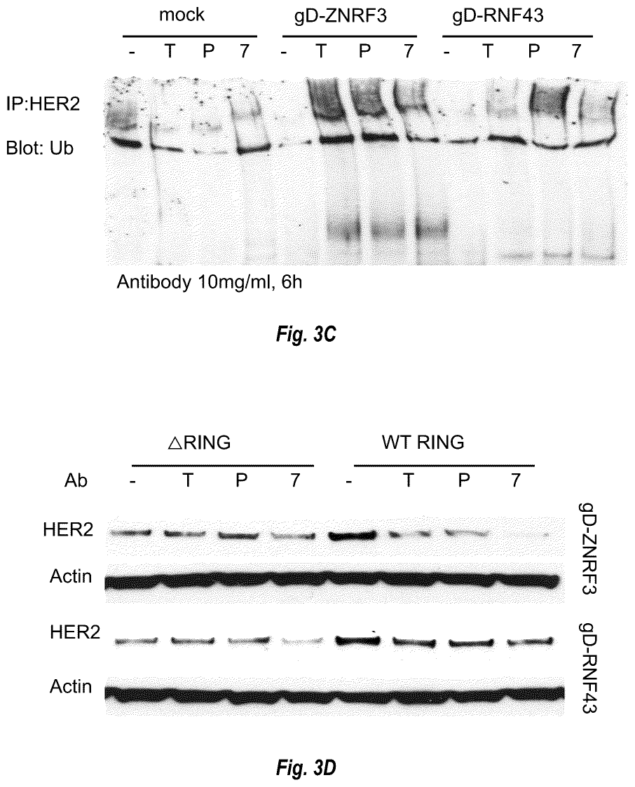

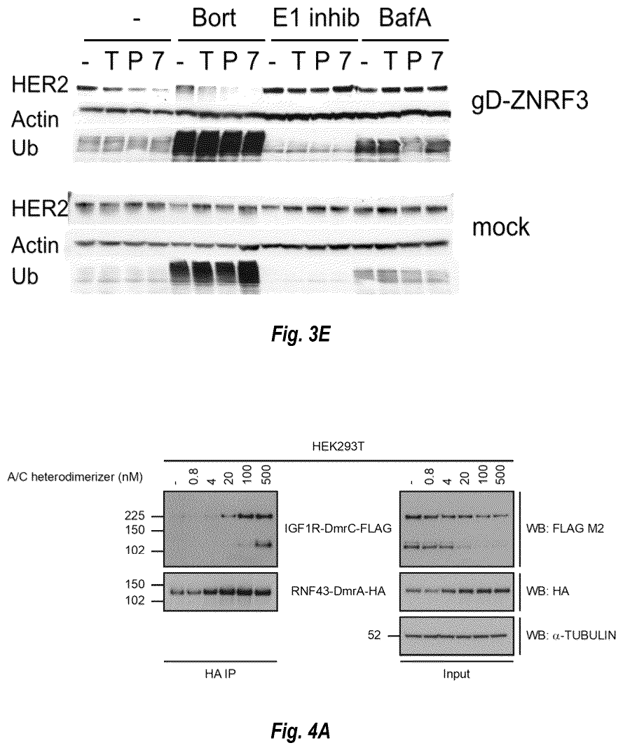

Bispecific antibodies were generated as described above targeting three different epitopes of HER2 (4D5, 7C2, and 2C4) (Fendly et al. Canc Res 1990) and either the gD epitope tag or an irrelevant antigen (human CD3). Impact of these bispecific antibodies was assessed on HT-29 cells expressing doxycycline inducible gD-tagged ZNRF3 or RNF43, as described above. Different antibodies showed different levels of clearance of HER2 from the cell surface (FIG. 3A). Monovalent binding of 2C4/CD3 was sufficient to drive partial clearance of HER2, though substantially less than is seen with the 2C4/gD bispecific Ab. To understand the impact of cell surface clearance on HER2 degradation we investigated HER2 levels by Western blot. Similar to what was observed by flow cytometry, ligase dimerization to HER2 was required for HER2 degradation. Importantly, monovalent binding to HER2, using anti-2C4/CD3 did not result in substantial HER2 degradation, suggesting that cell surface receptor clearance does not necessarily lead to receptor degradation and ligase dimerization is required (FIG. 3B). To substantiate that claim, we probed the ubiquitination status of HER2 through Immunoprecipitation. Ubiquitin smears were detected on HER2 after treatment with the various HER2/gD bispecific antibodies only in the presence of gD tagged ligases (FIG. 3C). Furthermore, ligase activity was required for HER2 degradation as deletion of the RING domain from either ZNRF3, and to a lesser extent RNF43, prevented HER2 degradation after ligase dimerization (FIG. 3D). Conversely, inhibition of the E1 Ubiquitin activating enzyme also rescued degradation, which we found was dependent on lysosomal rather than proteasomal activity (FIG. 3E).

### Example 11: Cell Surface Ligase Mediated Degradation is Applicable to Multiple Receptors

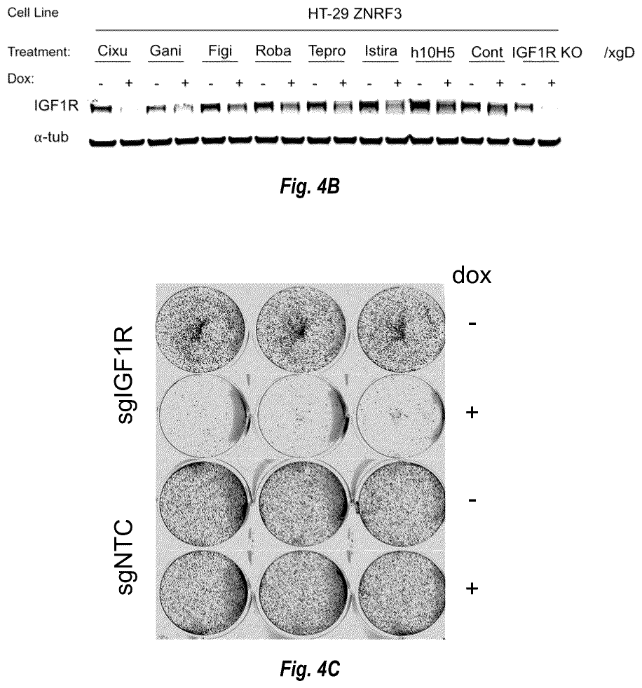

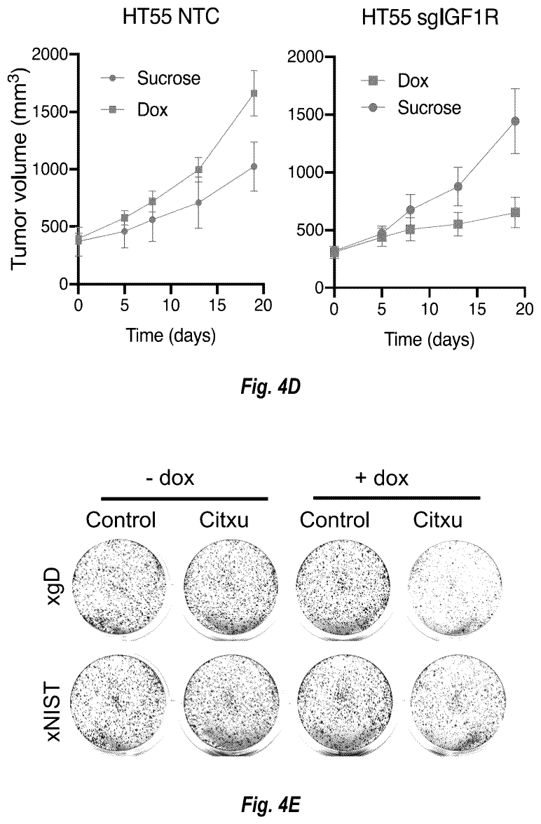

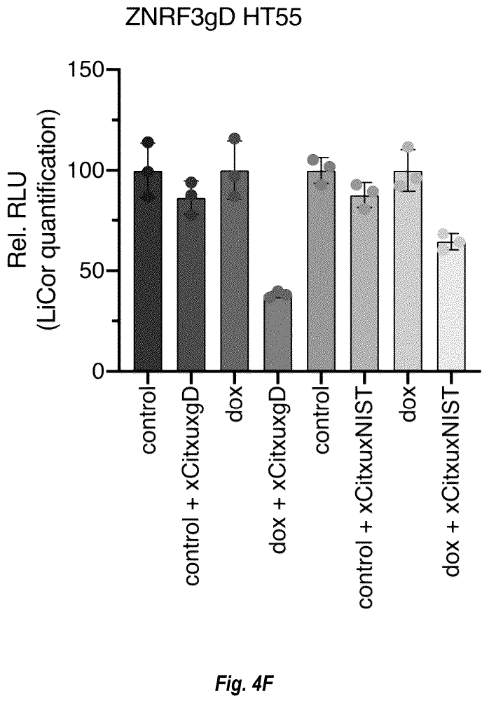

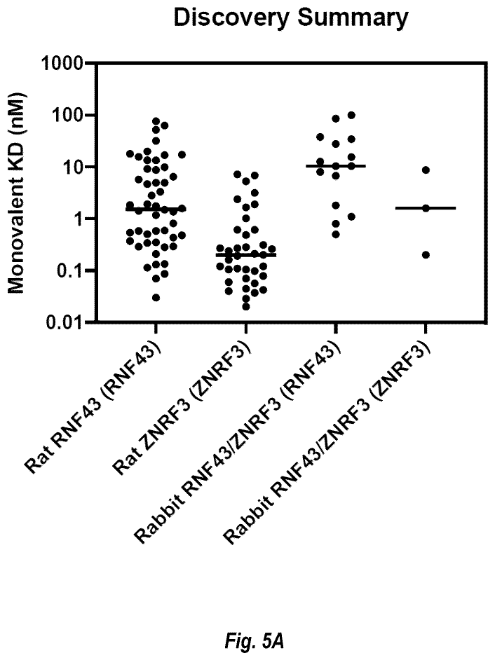

Chemical induced dimerization was used to probe the degradative potential of RNF43 to additional cell surface receptors, in particular IGF1R, which, like HER2, is a receptor tyrosine kinase. Similarly to what was observed with HER2, addition of the AC hetero-dimerizer to HEK293T cells transfected with a pBind plasmid co-expressing RNF43-DmrA-HA and IGF-1R-DmrC-FLAG, led to a dose dependent increase in binding between the two receptors (FIG. 4A left panel). Consequently, this increase in binding between ligase-target resulted in a dose dependent degradation of IGF-1R (FIG. 4A right panel). Bispecific antibodies were generated using variable regions targeting IGF-1R and gD and assessed for their ability to degrade IGF1R in HT-29 cells expressing a doxycycline inducible gD tagged ZNRF3. Western blot analysis of HT-29 cells treated with lug/ml of the depicted antibodies was performed 24 hr after treatment. For most bispecific antibodies, gD-ZNRF3 dimerization resulted in IGF1R degradation that was equivalent to the decrease in protein expression observed in a conditional KO made using CRISPr Cas9 and sgRNA specific for IGF1R (FIG. 4B). The phenotypic consequence of ligase mediated degradation of cell surface receptors was further evaluated using HT55 colon cancer cells. These cells rely on IGF1R for survival both in vitro (FIG. 4C) and in vivo (FIG. 4D). In line with the viability defect observed after genetic mediated KO of IGF-1R, HT55 cells treated for seven days with bispecific antibodies targeting IGF-1R and gD displayed a decrease in clonogenic growth, that was dependent on the expression of the ligase (FIG. 4E, 4F).

### Example 12: Kinetics of Clearance from the Cell Surface

Bispecific antibodies were generated using variable regions targeting IGF-1R (cixutumumab) and RNF43 (hSC37.39, US20170073430A1) and assessed for their ability to degrade IGF-1R in HT-29 cells with doxycycline induced overexpression of RNF43 by flow cytometry (FIG. 5D). One hour following antibody administration, cells treated with higher levels of the bispecific (0.1 ug/ml or greater) showed modestly higher levels of cell-surface IGF1R. By three hours, this trend had reversed with decreased cell surface IGF1R, and by eight hours the trend had saturated. Continuous treatment out to 96 hours showed no further clearance of target.

### Example 13: Assessment of RNF43 and ZNRF3 Bispecific Antibodies

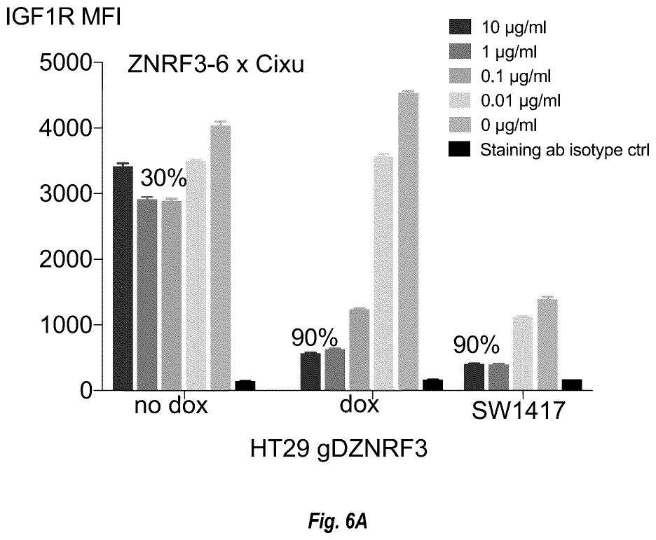

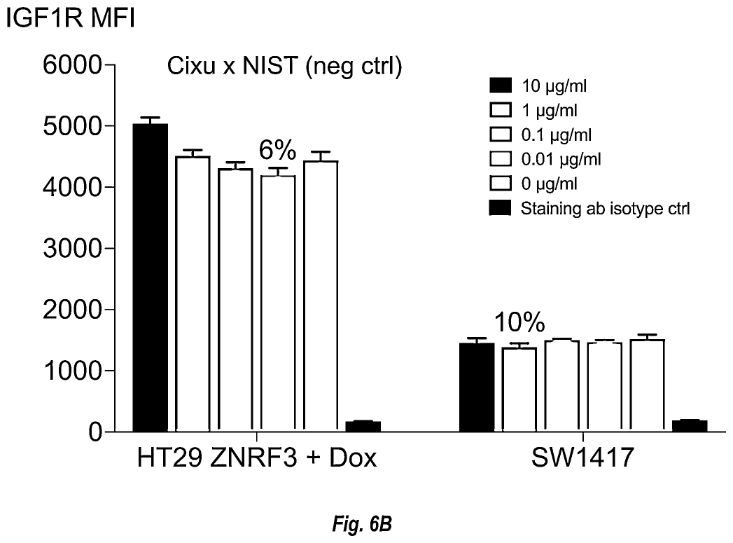

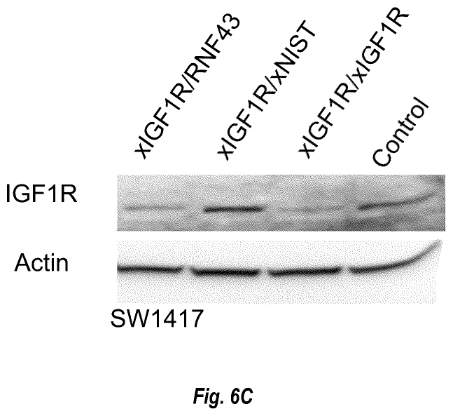

A subset of RNF43 and ZNRF3 antibodies discovered as described above were reformatted into bispecific antibodies targeting the respective ligases and IGF1R (cixutumumab) (i.e., “PROTABs”). Activity of these ligases was assessed by their ability to drive cell surface clearance of IGF1R from HT29 cells with doxycycline induced expression of the corresponding ligase by flow cytometry (FIG. 6A). Antibodies targeting the ligases with high monovalent affinities towards the ligases (greater than approximately 1 nM) showed saturating levels of IGF1R clearance when paired with cixutumumab, while lower affinity antibodies showed reduced activity (FIG. 6B). Further evaluation of the ZNRF3-IGF-1R PROTAB was done on SW1417 cells which express high endogenous level of ZNRF3 and RNF43. Flow cytometry revealed a dose dependent cell surface clearance of IGF-1R in these cells after treatment with a ZNRF3-IGF-1R antibody (FIG. 6A). Western blot confirmed cell degradation of IGF-1R in SW1417 after with an RNF43-IGF-1R antibody (FIG. 6C).

Without being bound by theory, the correspondence of ligase affinity to efficiency of degradation is hypothesized to be due to the durability of the ternary complex between IGF1R, the antibody and the ligase. Biochemical estimates suggested that at least four ubiquitins must be transferred to the target to enable efficient degradation, and thus a long-lived complex is necessary to enable multiple catalytic cycles. The durability of the overall complex was dictated by binding affinities of both sides of the bispecific antibody. For ligase antibodies much tighter than 1 nM, the affinity of cixutumumab (˜5 nM) was the primary determinant in the half-life of the complex, thus the efficiency of degradataion saturates. For ligase Abs weaker than 1 nM, we observed reduced clearance corresponding to the affinity of the ligase arm. Interestingly, no strong dependence of epitope was seen for the ligase antibodies, so for RNF43 and ZNRF3, the specific geometry of interaction did not strongly impact the efficiency of degradation. This is potentially different than what we saw for HER2 antibodies paired with an anti-gD Tag antibody (FIGS. 2A-2B) where changing the epitope of the HER2 targeted antibody appeared to influence degradation efficiency. Again without being bound by theory, we hypothesize this is due to the relatively micro-differentiations in epitope on the single extracellular protease-associated domain for the ligases, versus gross changes in epitopes targeting different domains for HER2. For bispecific ligase antibodies with other (non-cixutumumab) arms, the affinity at which this saturation takes place may thus vary corresponding to the affinity of that arm.

### Example 14: Impact of Bivalent Binding on Target Degradation

To investigate the impact of receptor clustering on target clearance, antibodies targeting two copies of the anti-RNF43 antibody or the anti-IGF1R antibody were generated in 2+1 Fab-IgG format (FIGS. 7A-7B). Activity of these antibodies was assessed by their ability to drive cell surface clearance of IGF1R from HT29 cells with doxycycline induced expression of the corresponding ligase by flow cytometry (FIGS. 7C-7F). Antibodies binding two copies of IGF1R and one copy of RNF43 saturated clearance at levels similar to the corresponding traditional knob-in-hole (1+1) bispecific antibody (FIG. 7C and FIG. 7E). Antibodies binding two copies of the ligase drove enhanced clearance compared to the corresponding traditional knob-in-hole (1+1) bispecifc antibody (FIG. 7D and FIG. 7F). These antibodies also reduced the level of cell-surface RNF43, presumably due to auto-ubiquitination in trans (data not shown).

### Example 15: Impact of Binding Distance on Target Degradation

We hypothesized that the distance between the ligase and target might influence the efficiency of target clearance. To investigate this phenomenon, bispecific antibodies in the one-armed FabIgG (OA-FabIgG) and one-armed FvIgG (OA-FvIgG) format were generated (FIG. 8A). Activity of these antibodies was assessed by their ability to drive cell surface clearance of IGF1R from HT29 cells with doxycycline induced expression of the corresponding ligase by flow cytometry (FIGS. 8B-8C). Both OA-FvIgG and OA-FvIgG formats resulted in enhanced degradation compared to the corresponding 1+1 bispecific. For the Fab-IgG format similar results were seen with both cixutumumab and isitiramab as the anti-IGF1R antibody. In addition, the results shown in FIG. 8 suggest that the efficiency of degradation can be fine-tuned or optimized by varying the location of each antigen binding domain, e.g, by placing the ligase antigen binding domain either in the external position (the Fv in OA-FvIgG or the Fab in OA-FabIgG) or the internal position.

### Example 16: Combination of Valency and Minimized Distance

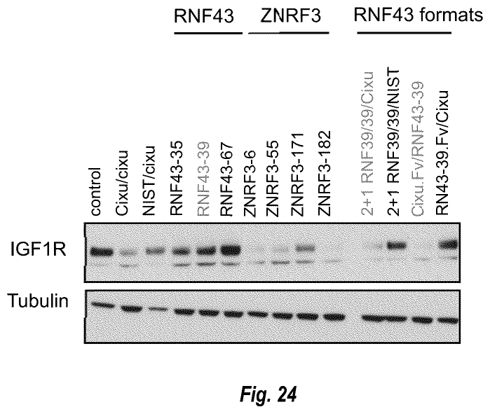

2+1 Fab-IgG and FvIgG antibodies comprised of 2×RNF37.39 and 1× cixutumumab are generated placing the cixutumumab Fab at the internal position. Assessment of these antibodies in the cell surface clearance assay described in Example 5 above finds that they clear IGF1R more efficiently than the corresponding traditional knob-in-hole (1+1) bispecific antibody. This data was further validated using DLD1 and looking at total IGF1R level using western blot analysis (FIG. 24).

### Example 17: Cell Surface Ligase-Mediated Clearance of EGFR

Multispecific antibodies were generated targeting EGFR (using cetuximab or D1.5 variable domains), and RNF43 or an irrelevant antigen. Activity of these antibodies was assessed by their ability to drive cell surface clearance of EGFR from HT29 cells with doxycycline induced expression of the corresponding ligase by flow cytometry (FIG. 9). Both 1+1 bispecific antibodies targeting both the ligase and EGFR resulted in efficient clearance. Consistent with the results described in Example 14, the 2+1 FabIgG targeting 2×RNF43 and 1×EGFR resulted in more complete clearance.

### Example 18: Identification and Characterization of Novel Cell Surface Ligases

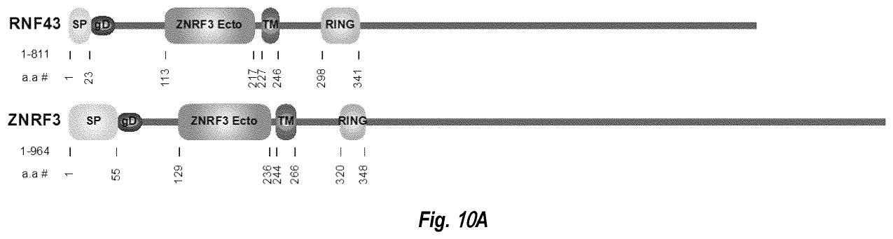

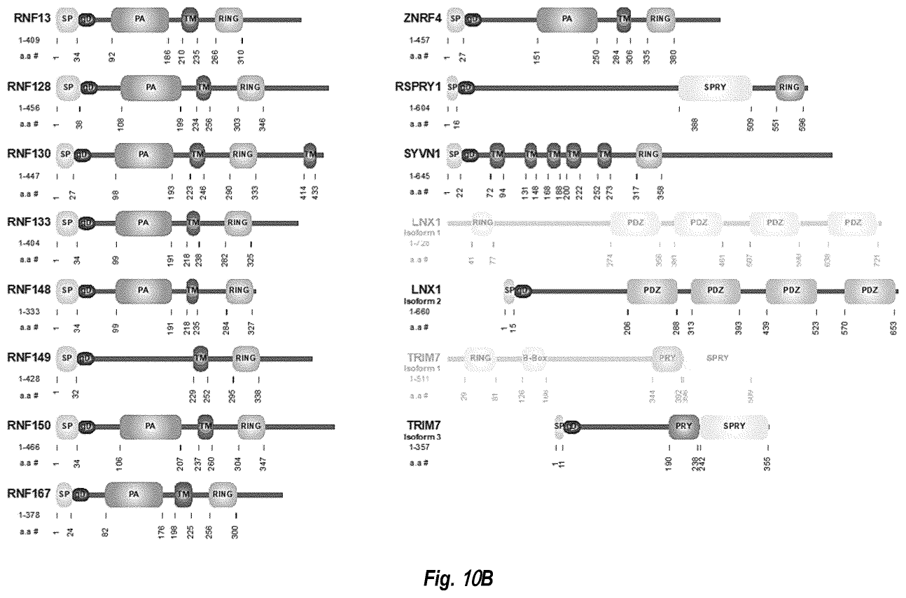

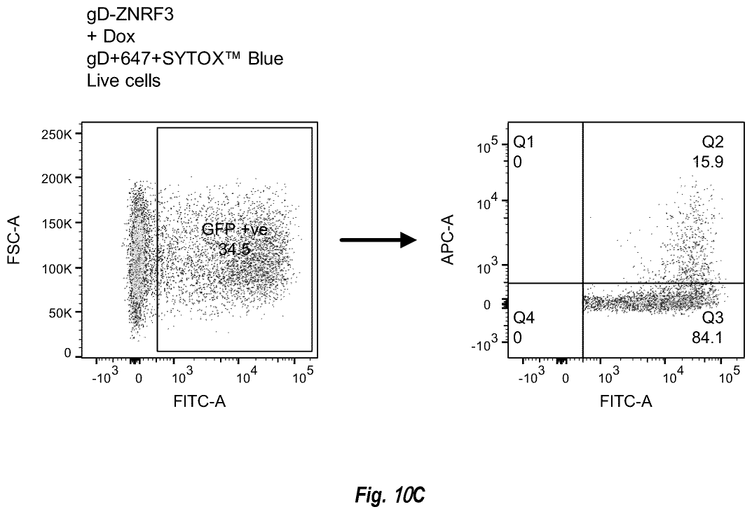

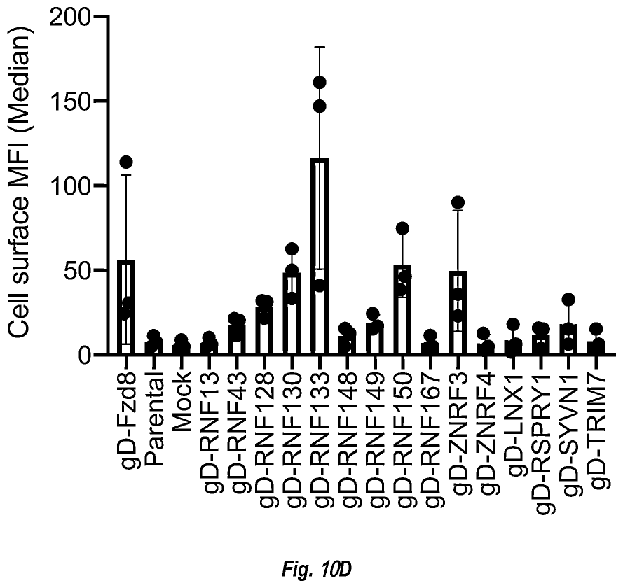

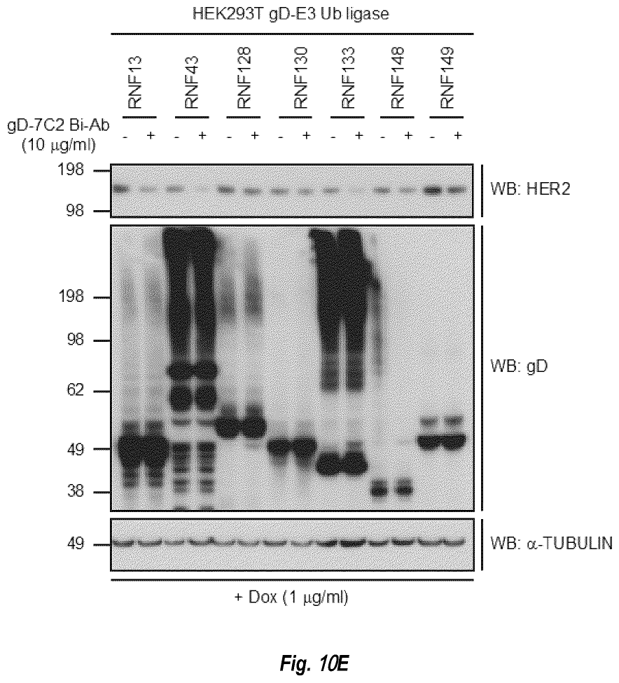

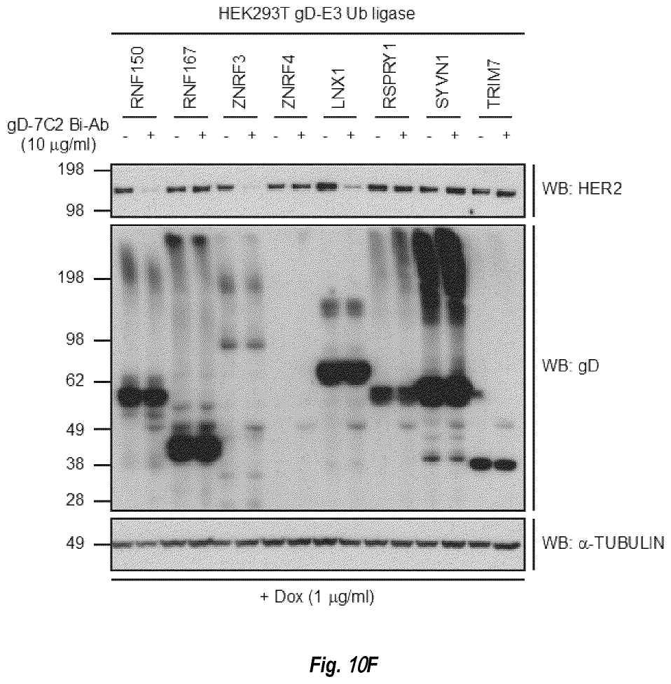

To identify putative cell surface ligases the domain structure of RNF43 and ZNRF3 was evaluated (FIG. 10A). This revealed the presence of an N-terminal Signal Peptide (SP) that has been associated with membrane integration. As such, a list of known E3 ubiquitin ligases was evaluated using both UNIPROT and the Signal-P software to identify ligases with SP. Among the identified proteins, we evaluated the following 15 ligases which include both single and multiple transmembrane domain ligases: RNF13, RNF43, RNF128, RNF130 (having two transmembrane domains, FIG. 10B), RNF133, RNF148, RNF149, RNF150, RNF167, ZNRF3, ZNRF4, LYNX1, RSPRY1, SYVN1 (having five transmembrane domains, FIG. 10B), and TRIM7.

Doxycycline-inducible pBind plasmids with IRES-eGFP encoding all 15 ligases with an N-terminal gD tag were generated (FIG. 10B). Cell surface presentation of each ligase was evaluated using FACS analysis. Briefly, HEK293T cells were transiently transfected with the respective ligase plasmids. After 24 hours cells were treated with 1 ug/ml doxycycline for an additional 24 hours to induce ligase expression. Live cells were then prepared for FACS analysis by staining using an anti-gD primary antibody followed by an APC secondary antibody. To evaluate doxycycline induction the IRES-eGFP signal was examined (FIG. 10C). The APC median florescence intensity (MFI) was also quantified from three independent experiments for each sample as outlined (FIG. 10D). As assay controls, parental and mock transfected cells were also evaluated as well as HEK293T cells constitutively expressing gD-tagged Fzd8, a known cell surface protein.

In parallel to the FACS analysis, HEK293T transfected cells were also used to evaluate the degradative potential of each putative cell surface ligase. Following 24 hours doxycycline induction, cells were treated with 10 μg/ml gD+HER2 (7C2) bispecific antibody for 24 hours. Cells were then lysed and prepared for Western blot analysis to examine the total levels of HER2 upon bispecific antibody treatment. (FIGS. 10E and 10F.) Assessment of these cell surface ligases in the standard cell degradation assay finds that numerous cell surface ligases can be dimerized to HER2 and lead to HER2 efficient degradation.

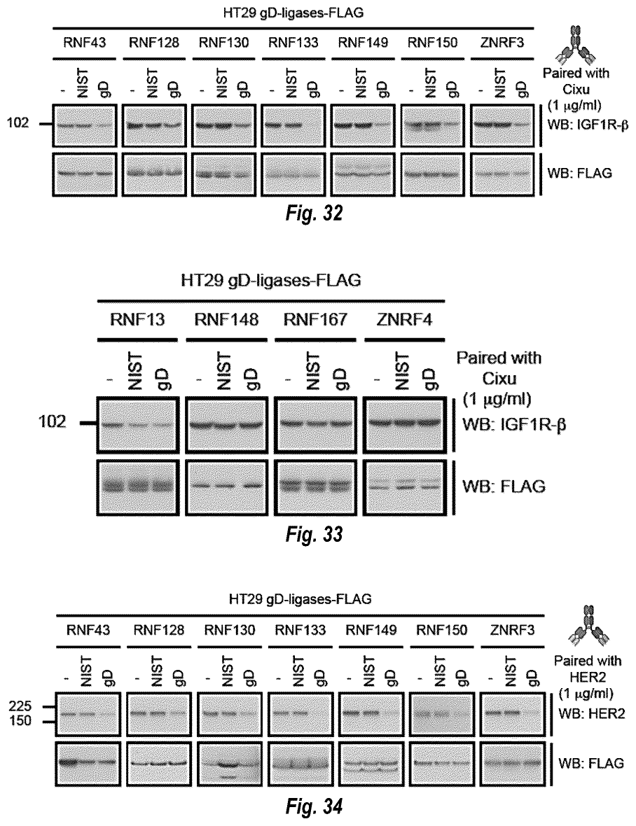

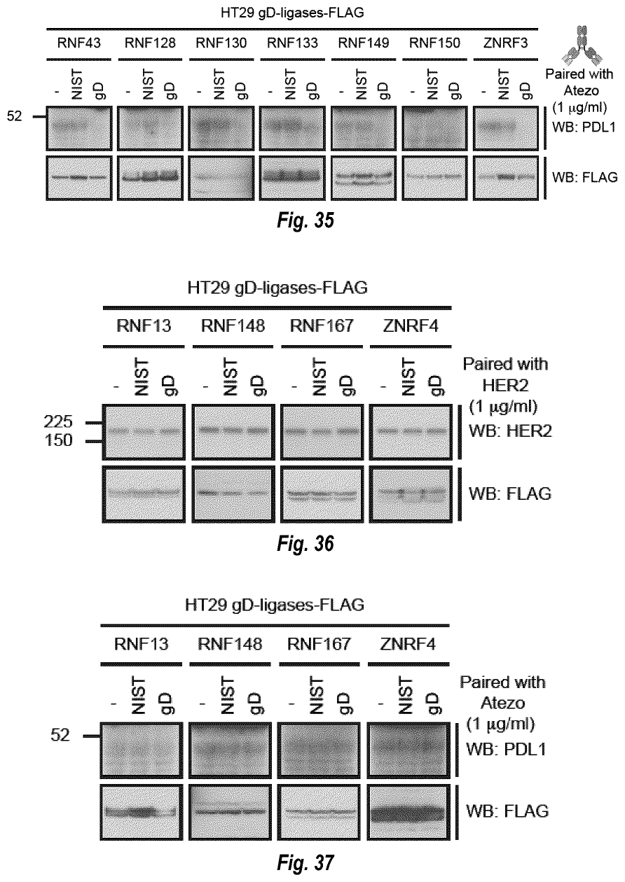

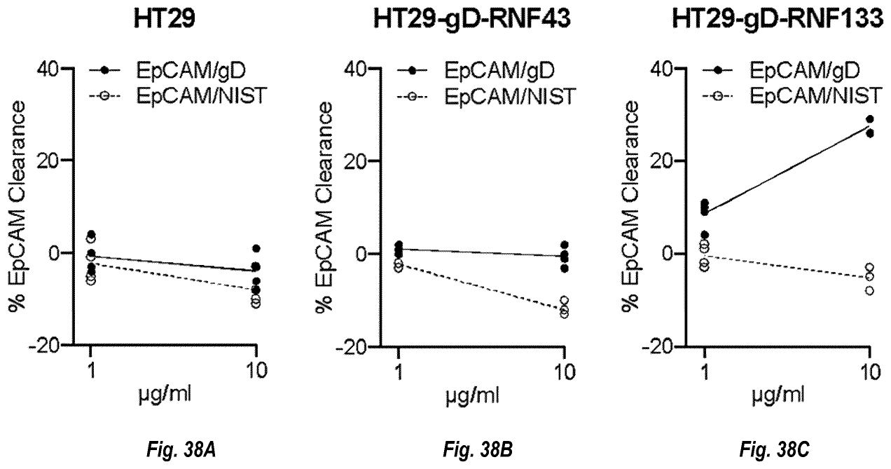

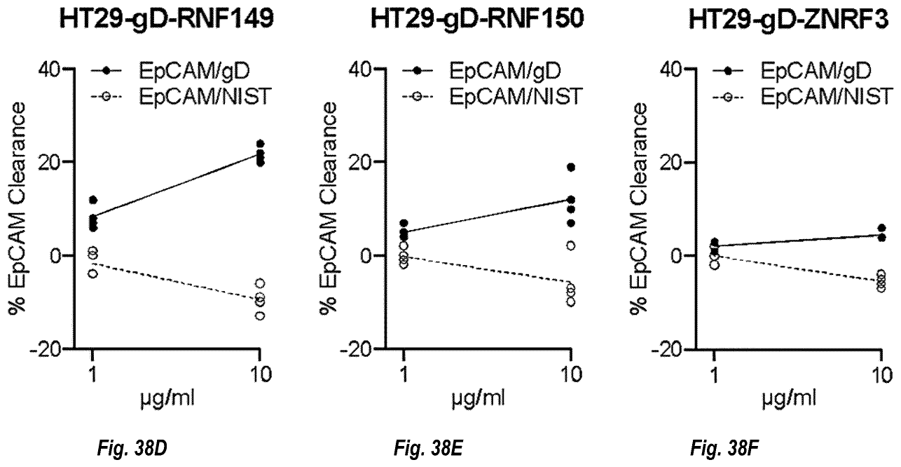

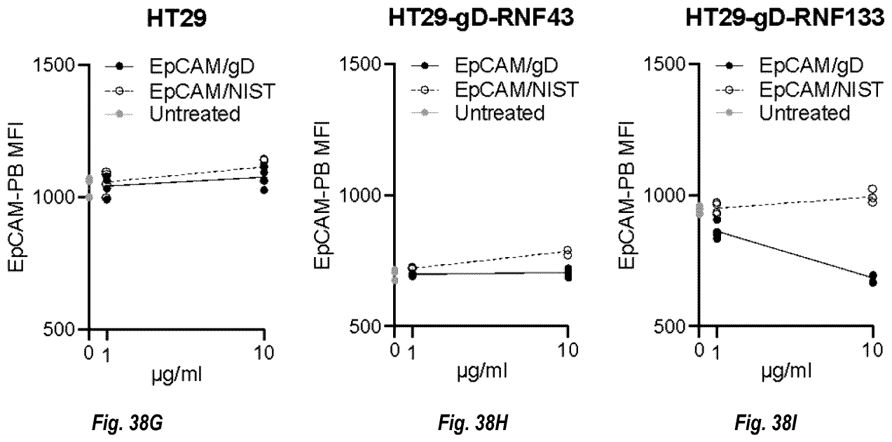

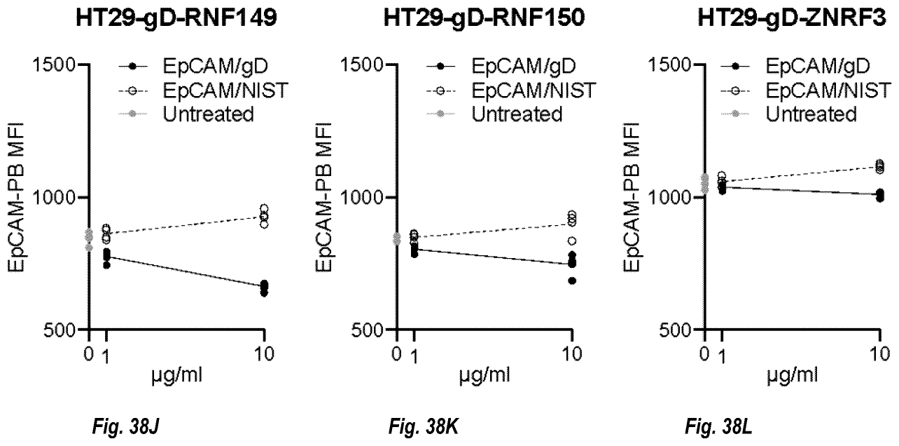

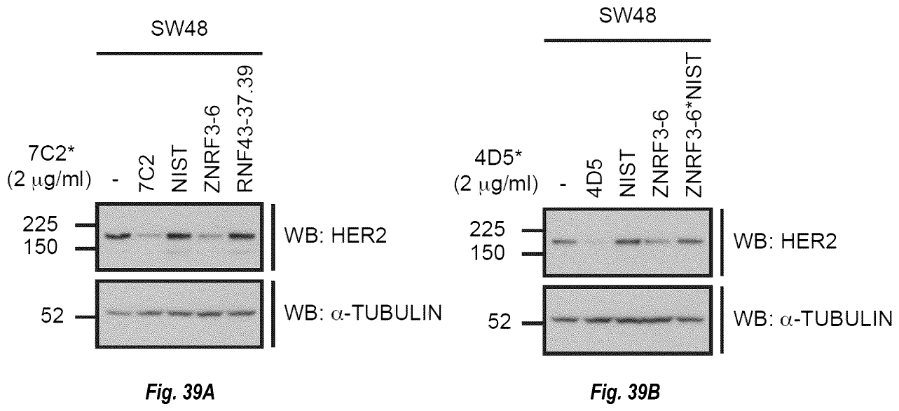

Additional experiments were carried out as follows. FIG. 10D shows that RNF128, RNF130, RNF133, RNF149 and RNF150 exhibited detectable cell surface expression while RNF13, RNF148, and RNF167 expression was substantially lower. To assess whether cell-surface expression broadly enabled degradation of non-natural targets, we treated cells with gD*IGF1R PROTABs, and monitored IGF1R levels. Western blot analysis confirmed that colocalizing validated cell surface E3 ubiquitin ligases to IGF1R drives degradation, as shown in FIG. 32. Conversely, ligases with undetectable cell surface expression by flow cytometry were unable to induce target degradation as shown in FIG. 33. We also generated bispecific antibodies that target gD and either HER2 or programmed death-ligand 1 (PD-L1), two therapeutically relevant cancer targets. Similar to IGF1R, degradation of HER2 and PD-L1 occurred upon engagement of various ligases as shown in FIGS. 34 and 35, but no degradation was detected using ligases with undetectable cell surface expression as shown in FIGS. 36 and 37.

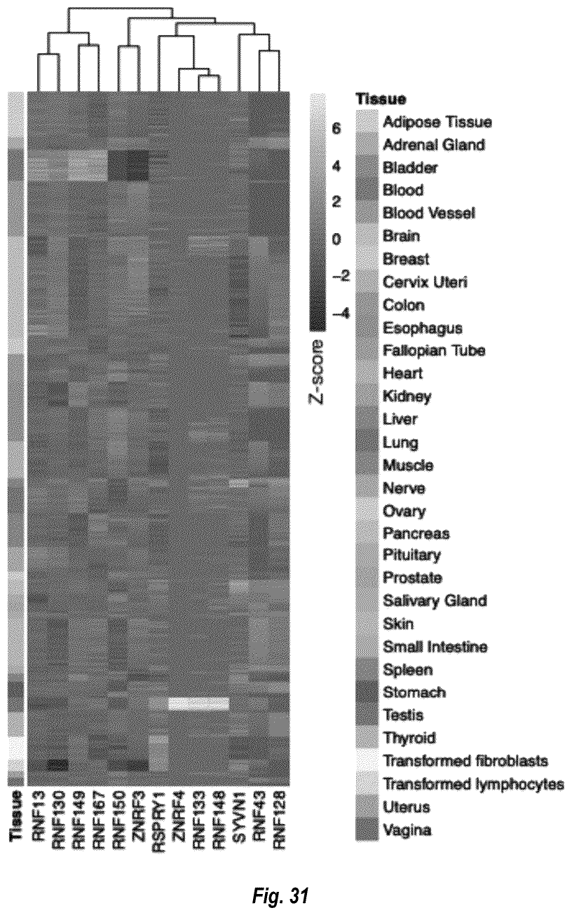

FIG. 31 shows that several of the newly identified cell surface E3 ligases showed discrete tissue expression patterns, providing possibilities for tissue specific degradation based on choice of particular E3 ligase. For example, RNF130, RNF149 and RNF167 displayed apparent elevated expression in the hematopoietic compartment, and these ligases could be used to target cells of hematopoietic origin, while RNF133 and RNF148 expression was mainly restricted to the testis and these ligases could be used to target testicular cells.

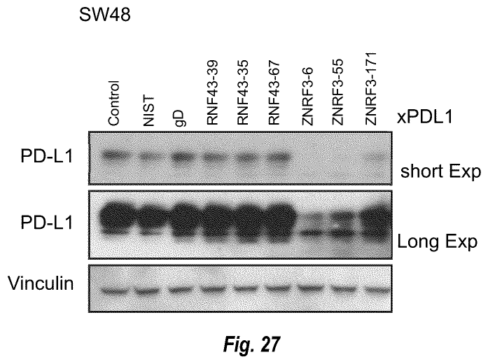

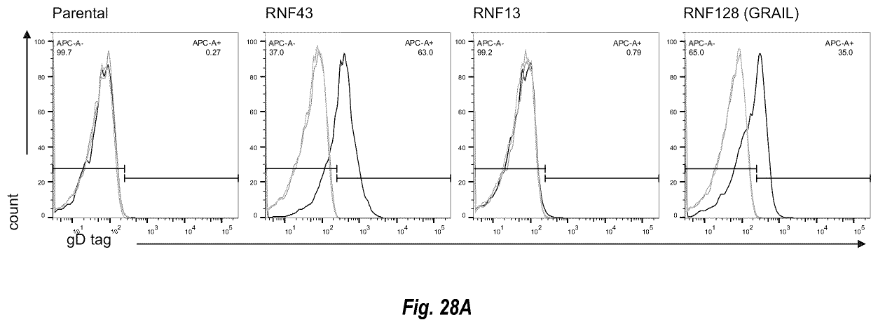

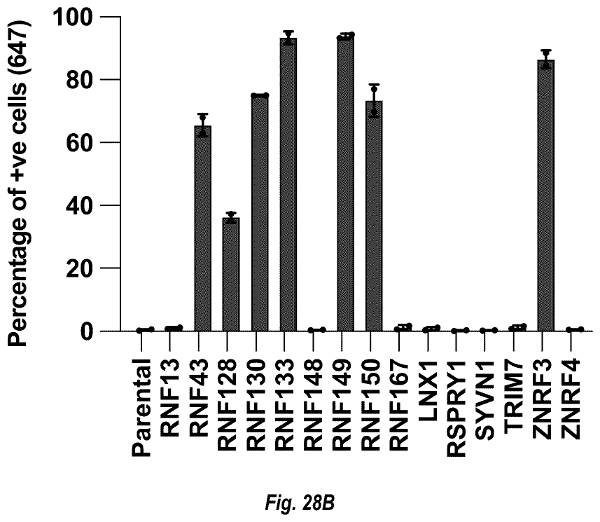

The catalytic activity of an E3 ubiquitin ligase is considered essential to facilitate the final transfer of ubiquitin from the ubiquitin-conjugating enzymes (E2s) to substrates to alter that substrate function. To test the importance of catalytic activity, catalytically active and inactive versions of each individual ligase can be used in degradation assays to assess whether or not catalytic activity is required for each ligase to mediate degradation of cell surface receptors. Preliminary data suggests that certain ligases may require catalytic activity while other certain ligases may not. A separate cell line was used to validate cell surface expression of newly identified ligases. HT29 cells were transfected with each individual ligase, selected with puromycin for stable integration. Stable lines were treated with 1 ug/ml doxycycline for an 24 hours to induce ligase expression. Live cells were then prepared for FACS analysis by staining using an anti-gD primary antibody followed by an APC secondary antibody. The percentage of APC positive cells was also quantified from three independent experiments for each sample as outlined (FIG. 27A, B).

### Example 19: Cell Surface Clearance of Diverse Substrates

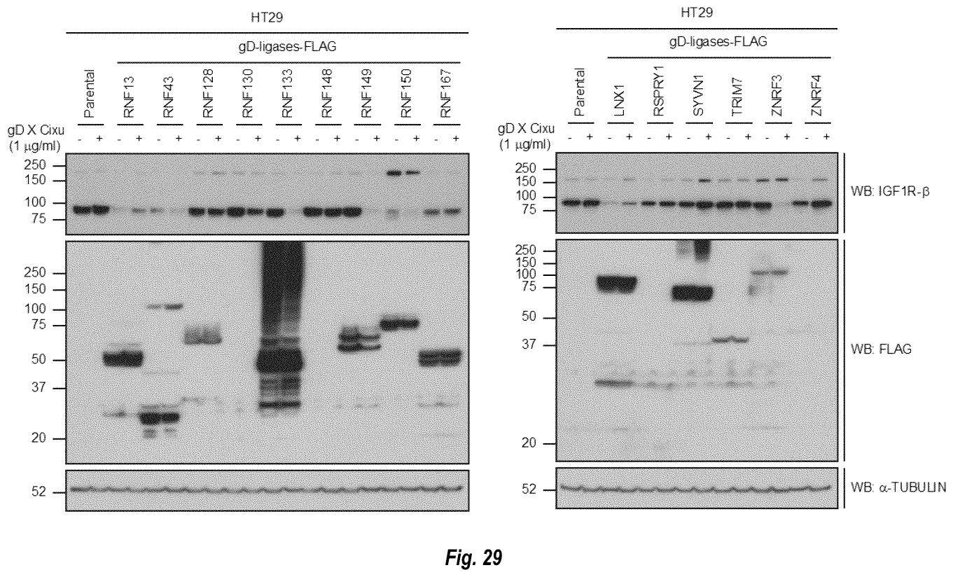

Bispecific antibodies are generated targeting the gD epitope and exemplary receptor tyrosine kinases, HER2, IGF1R, and EGFR, as well as various structurally distinct cell-surface proteins, including multipass transmembrane domain proteins, e.g., FZLD5, proteins having large extracellular and intracellular domains, e.g., EpCAM, and proteins having an immunoglobulin domain with a short intracellular domain, e.g, PD-L1. Antibodies at 10 ug/ml are added to cells overexpressing ligases tagged with an N-terminal gD tag. Western blot analysis reveals addition of the bispecific antibody drives degradation of the target antigen. An example is depicted in FIG. 29 using HT29 cells that stably express various cell surface ligases. Dimerization of several ligases to IGF1R using gD/IGF1R antibody result in profound receptor degradation.

### Example 20: Assessing Wnt Signaling Pathway Activation by TOPBrite TCF Reporter Assay

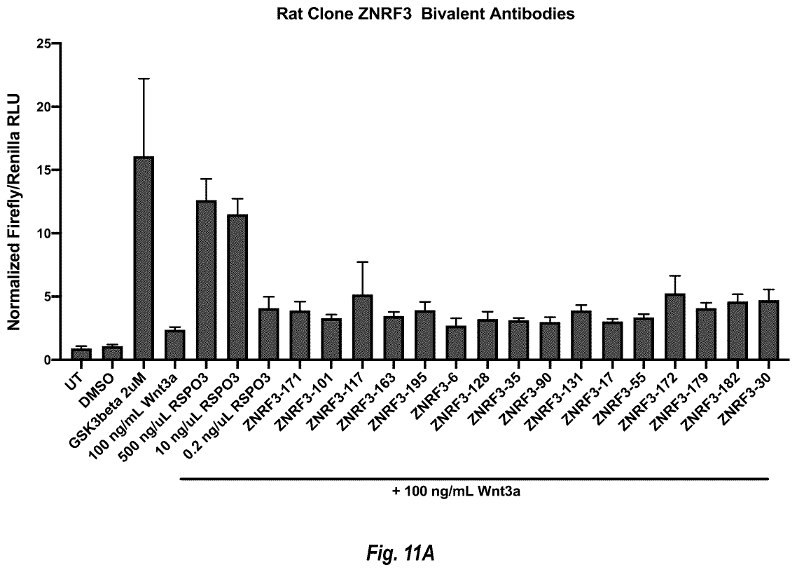

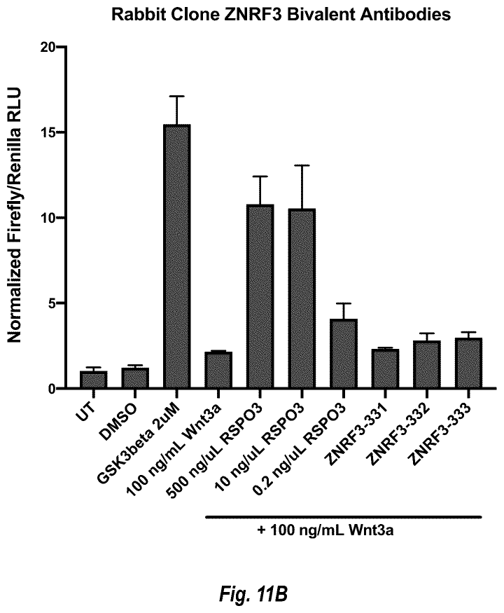

The Wnt-dependent reporter Nano-Glo Dual® luciferase system (Promega), containing a control promotor that expresses Renilla luciferase and a TCF promotor that expresses firefly luciferase, was used to evaluate the activation of Wnt/β-Catenine by rat or rabbit multispecific antibodies targeting ZNRF3. HEK293 cells transiently transfected with plasmids containing a control promoter and TCF promoter were plated in Corning® 96-well Flat Clear Bottom White Polystyrene TC-treated Microplates at 100,000 cells per well and incubated in RPMI media with 10% FBS, 1% penicillin-streptomycin, 1×L-glutamine and 40 μg/mL hygromycin B (Thermo) overnight. On the following day, rat or rabbit clone ZNRF3 antibodies at 0.1 mg/mL and recombinant human Wnt3a at 100 ng/mL (Abcam) were added to the cells. GSK3 inhibitor at 2 uM or RSPO3 at 500, 10 and 0.2 ng/μL in the presence of 100 ng/nL Wnt3a was used as a positive control. DMSO or 100 ng/mL Wnt3a treated cells were negative controls. Upon 24 hours (h) incubation, cell media was replaced with fresh media and an equal volume of Dual-Glo® reagent was added to each well followed by gentle mixing. After 10 minutes incubation, the firefly luminescence signal was measured using GloMax® Discover Microplate Reader at 0.3 second integration time (Promega). Then an equal volume of Dual-Glo Stop&Glo® reagent was added to each well and the Renilla luminescence signal was measured after 10 minutes incubation using the same setting. The relative ratio was calculated from the ratio of firefly to Renilla luminescence and then further normalized by taking a ratio of an untreated sample. Results from ZNRF3 antibodies are shown in FIGS. 11A-11B.

### Example 21: Assessing IGF1R Cell-Surface Clearance by NanoLuc® Luciferase Complementation Assay

A HiBiT tag was introduced to the N-terminus of IGF1R by a CRISPR-Cas9 system on HT29 cells. Single clones with the highest NanoLuc® luciferase luminescence readout were selected and verified by Sanger sequencing. Cells were plated onto Corning® 96-well Flat Clear Bottom White Polystyrene TC-treated Microplates at 100,000 cells per well and incubated in ATCC recommended media in the presence of various concentrations of antibodies. After 24 hours (or the indicated period of time), the cells were washed with 100 μL PBS once and then replenished with 100 μL fresh media. The detection reagent was prepared by diluting the LgBiT protein at a ratio of 1:100 and the substrate at a ratio of 1:50 into a desired volume of detection buffer supplied in the detection kit. A 100 μL of the detection reagent then was added to the cells in 100 uL fresh media. For Nano-Glo® HiBiT Extracellular Detection System which detects the surface level of IGF1R, detection was performed upon 10 min incubation at room temperature with gentle mixing using a plate shaker. Percent IGF1R clearance was calculated using the equation % Clearance=(Untreated samples RLU-treated samples RLU)/Untreated samples RLU*100. RNF43 and ZNRF3 multispecific antibodies were assessed in this cell line as well (Tables 5-7). A subset of samples were run in two separate runs, in which case both results are included.

TABLE 5
Surface IGF1R clearance by bispecific RNF43-IGF1R antibodies

| | % IGF1R clearance |
|---|---|
| Short Name | at 1 ug/ml |
| cixu/RNF43-71 | 54% |
| cixuRNF43-25 | 61% |
| cixu/RNF43-33 | 25% |
| cixu/RNF43-35 | 73% |
| cixu/RNF43-90 | 72% |
| cixu/RNF43-69 | 42% |
| cixu/RNF43-75 | 52% |
| cixu/RNF43-31 | 60% |
| cixu/RNF43-41 | 50% |
| cixu/RNF43-80 | 60% |
| cixu/RNF43-130 | 71% |
| cixu/RNF43-177 | 42% |
| cixu/RNF43-104 | 44% |
| cixu/RNF43-156 | 62% |
| cixu/RNF43-168 | 69% |
| cixu/RNF43-176 | 16% |
| cixu/RNF43-170 | 33% |
| cixu/RNF43-221 | 7% |
| cixu/RNF43-180 | 51% |
| cixu/RNF43-107 | 48% |
| cixu/RNF43-201 | 24% |
| cixu/RNF43-106 | 21% |
| cixu/RNF43-210 | 22% |
| cixu/xRNF43 37.39 | 40% |
| cixu/RNF43-90 | 77% |
| cixu/RNF43-75 | 28% |
| cixu/RNF43-61 | 29% |
| cixu/RNF43-31 | 51% |
| cixu/RNF43-41 | 30% |
| cixu/RNF43-80 | 47% |
| cixu/RNF43-86 | 36% |
| cixu/RNF43-53 | 66% |
| cixu/RNF43-186 | 50% |
| cixu/RNF43-181 | 76% |
| cixu/RNF43-196 | 67% |
| cixu/RNF43-216 | 65% |
| cixu/RNF43-130 | 71% |
| cixu/RNF43-116 | 50% |
| cixu/RNF43-104 | 38% |
| cixu/RNF43-145 | 74% |
| cixu/RNF43-156 | 77% |
| cixu/RNF43-224 | 70% |
| cixu/RNF43-176 | 63% |
| cixu/RNF43-200 | 68% |
| cixu/RNF43-206 | 19% |
| cixu/RNF43-220 | 63% |
| cixu/RNF43-128 | 4% |
| cixu/RNF43-126 | 62% |
| cixu/RNF43-217 | 61% |
| cixu/RNF43-170 | 33% |
| cixu/RNF43-179 | 27% |
| cixu/RNF43-221 | 23% |
| cixu/RNF43-180 | 66% |
| cixu/RNF43-129 | 61% |
| cixu/RNF43-107 | 69% |
| cixu/RNF43-117 | 38% |
| cixu/RNF43-152 | 20% |
| cixu/RNF43-201 | 26% |
| cixu/RNF43-106 | 18% |
| cixu/RNF43-123 | 24% |
| cixu/RNF43-210 | 9% |
| Cixutumumab/RNF43.hSC37.39 | 68% |
| RNF43.37.39/NIST | −9% |
| Cixu/NIST | 12% |

TABLE 6
Surface IGF1R clearance by bispecific ZNRF3-IGF1R antibodies

| | % IGF1R clearance |
|---|---|
| Short Name | at 1 ug/ml |
| cixu/ZNRF3-172 | 60% |
| cixu/ZNRF3-182 | 60% |
| cixu/ZNRF3-279 | 34% |
| cixu/ZNRF3-314 | 51% |
| cixu/ZNRF3-269 | 49% |
| cixu/ZNRF3-219 | 9% |
| cixu/ZNRF3-300 | 50% |
| cixu/ZNRF3-222 | 21% |
| cixu/ZNRF3-322 | 59% |
| cixu/ZNRF3-270 | 66% |
| cixu/ZNRF3-237 | 3% |
| cixu/ZNRF3-171 | −3% |
| cixu/ZNRF3-55 | 55% |
| cixu/ZNRF3-6 | 52% |
| Cixu/gD | −42% |
| Cixu/NIST | 2% |

TABLE 7
Surface IGF1R clearance by new format bispecific antibodies

| | % IGF1R clearance |
|---|---|
| Short Name | at 1 ug/mle |
| 2 + 1 Cixu-Cixu/RNF43.37.39 | 49% |
| 2 + 1 Cixu-Cixu/NIST | 37% |
| 2 + 1 Istira-Istira-RNF43.37.39 | 49% |
| 2 + 1 Istira-Istira/NIST | 7% |
| 2 + 1 RNF43-RNF43.39.39/Cixu | 57% |
| 2 + 1 RNF43-RNF43.39.39/Istira | −5% |
| 2 + 1 RNF43-RNF43.39.39/NIST | 47% |
| RNF43-RNF43.hsc37.39 hIgG1 | 3% |
| Cixu-RNF43 Fv-IgG | 75% |
| RNF43-Cixu Fv-IgG | 30% |
| Istira-RNF43 Fv-IgG | 71% |
| RNF43-Istira Fv-IgG | 40% |
| xIGF1R.Cixu.hIgG1 | 18% |
| xIGF1R.Istira.hIgG1 | 41% |

Tables 5-7 show the percent surface IGF1R-HibiT clearance. Table 5 shows that rat Cixu/RNF43 bispecific antibodies triggered cell surface IGF1R-HibiT clearance in HT29 at a concentration of 1 μg/mL. Table 6 shows that rat Cixu/ZNRF3 bispecific antibodies triggered cell surface IGF1R-HibiT clearance in HT29 at a concentration of 1 μg/mL. Table 7 shows that new format antibodies including Fv-IgGs and 2+1 trispecific format triggered cell surface IGF1R-HibiT clearance in HT29 at a concentration of 1 μg/mL.

### Example 22: Monitoring IGF1R Surface Clearance Kinetics Using NanloLuc® Luciferase Complementation Assay

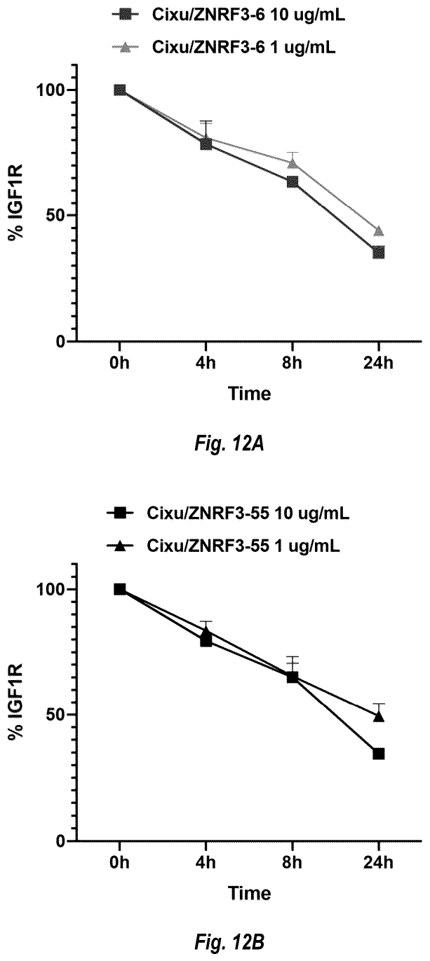

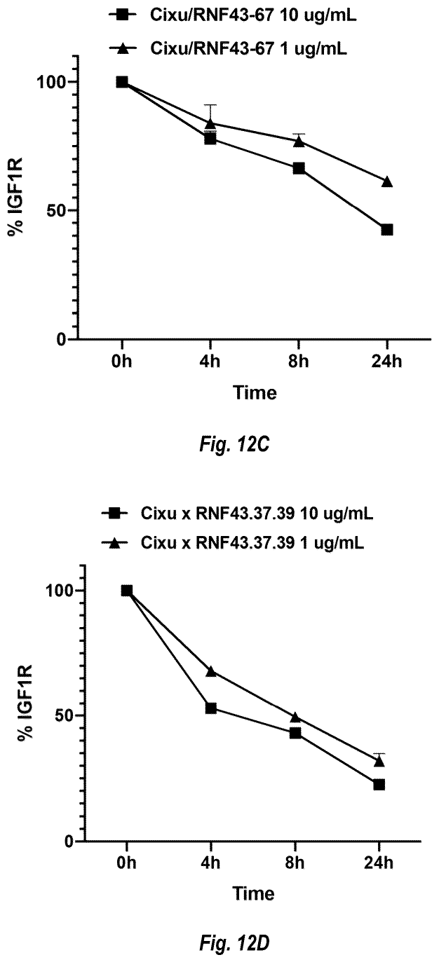

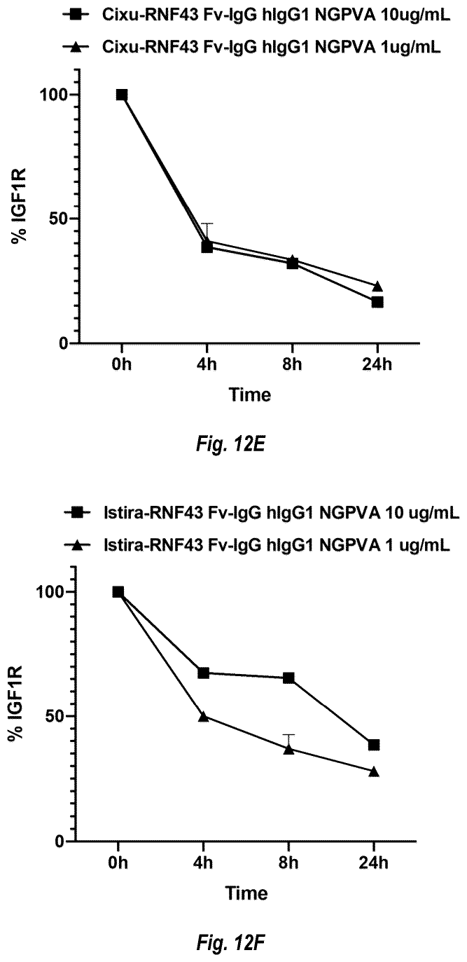

HibiT tagged IGF1R HT29 cells were seeded overnight at 100,000 cells per well in a Corning® 96-well Flat Clear Bottom White Polystyrene TC-treated Microplates and incubated in ATCC recommended media. 1 μg/mL or 10 μg/mL bispecific and new format antibodies were added to the cells on the second day and incubated at various time points: 0, 4, 8, 24 hours. The luminescence signal of IGF1R-HibiT was detected using the HibiT extracellular system following the description written in the previous example. Percent IGF1R was calculated using the equation % IGF1R=(1−(Untreated samples RLU−treated samples RLU)/Untreated samples RLU)*100. Assessment of a variety of multispecific antibodies showed different rates of degradation (FIGS. 12A-12F).

### Example 23: Multiplex NanoLuc Luciferase Complementation Assay with Cell Viability Assay

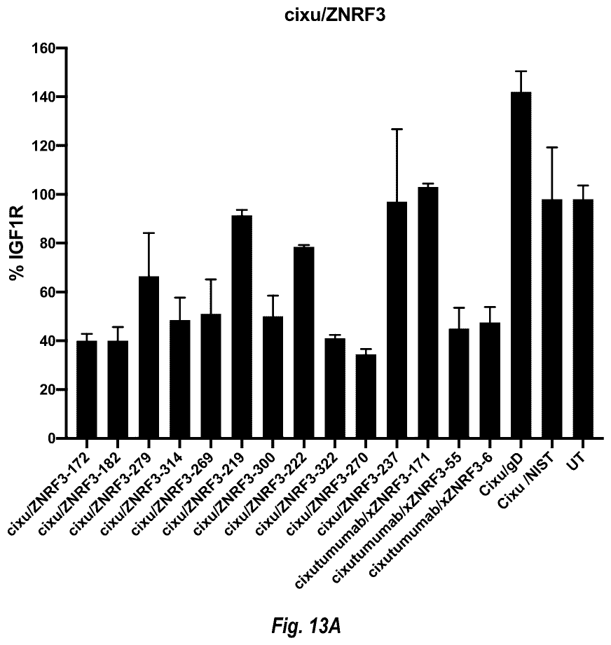

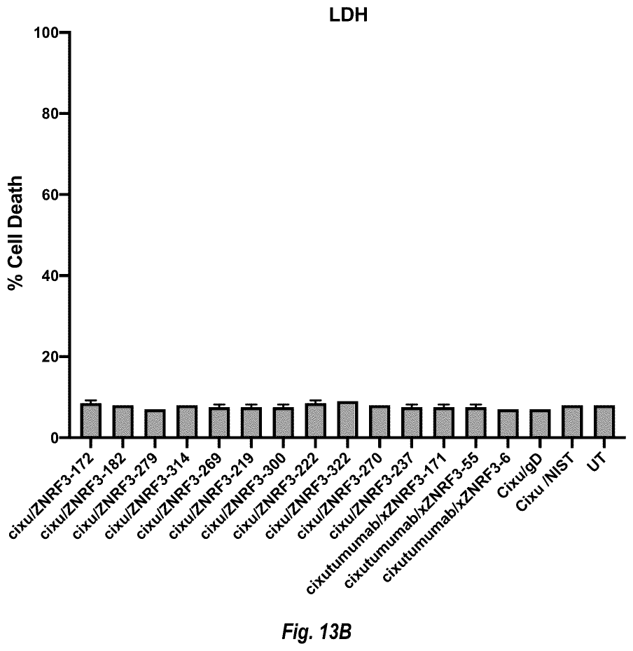

HiBiT tagged IGF1R HT29 cells were seeded overnight at 100,000 cells per well in a Corning® 96-well Flat Clear Bottom White Polystyrene TC-treated Microplates and incubated in ATCC recommended media. 1 ug/mL bispecific antibodies were added to the cells on the second day and incubated for 24 h. Cell media containing lactate dehydrogenase were collected for LDH cytotoxicity assay (Promega) before the addition of the HiBiT extracellular detection reagent. About 2-5 μL of the media was diluted 300× in LDH storage buffer and then mixed with an equal volume of LDH detection reagent. LDH lytic reagent was added to the control samples for maximum LDH detection. The reaction was incubated at room temperature for 30-60 minutes and the luminescence signal was measured using GloMax® Discover Microplate Reader at 1 second integration time. The % cytotoxicity was calculated using the equation: % Cytotoxicity=100×((Experiment LDH release−Medium background)/(Maximum LDH release control-Medium background)). Post HiBiT extracellular detection, cells were washed twice with PBS and replenished with fresh media., CellTiter-Glo® reagents (Promega) was then added to the cells in an equal volume and incubated for 10-30 min before the luminescence detection using GloMax® Discover Microplate Reader at 1 second integration time (Promega). While various multi-specifics showed variable influences on target degradation, no impact was seen on cell death as determined by LDH assay or the number of cells as determined by Cell Titer-go (FIGS. 13A-13C).

### Example 24: Assessing IGF1R Degradation Using NanoLuc® Luciferase Complementation Assay

HibiT tagged IGF1R HT29 cells were seeded overnight at 100,000 cells per well and then treated with various bispecific and new format antibodies at 1 ug/mL. Upon 24 h incubation the cells were incubated with the HibiT lytic detection reagent for the total amount of IGFR-HibiT. The lytic detection reagent was prepared by diluting the LgBiT protein at a ratio of 1:100 and the substrate at a ratio of 1:50 into a desired volume of detection buffer supplied in the detection kit. The luminescent signal was measured using GloMax® Discover Microplate Reader at 1 second integration time after 20 minutes incubation with the lytic detection reagent. Percent IGF1R was calculated using the equation mentioned in the previous example (FIG. 14A). Western blot was also used to determine the total amount of IGF1R after 24 h antibody treatment. HT29 IGF1R-HiBiT tagged and wild type cells (WT) were treated with various antibodies as indicated (1. Cixu-RNF43 Fv-Ig, 2. Cixutumumab/RNF43.hSC37.39, 3. Cixu/NIST, 4. Cixu/Cixu) at 1 μg/mL for 24 h. Untreated cells (5. UT) were used as a control. 20 μg of total proteins were loaded in each lane and the target protein is probed with anti IGF1 receptor antibody (Abcam EPR19322) which recognizes both pro IGF1R (˜200 kDa) and IGF1R (˜95 kDa). j-actin is shown as a protein loading control (FIG. 14B). Western blot analysis shows that the HiBit line has predominantly Pro-IGF1R which is intracellular and not subject to degradation, resulting in limited degradation in the lytic Hibit assay. The extracellular mature IGF1R is more abundant in the WT HT-29 cells and is efficiently degraded by the ligase bispecifics as shown by Western blot.

### Example 25: Trispecific Antibodies for Enhanced Degradation of IGF1R and EGFR

Preliminary data showed that the combination of two traditional knob-in-hole bispecifics (RNF43/Cixu and ZNRF3/Cixu bispecifics) results in increased clearance. Because it would be advantageous, e.g., for ease of manufacture and administration to subjects, to create a single molecule having all the desired antigen binding domains, the 2+1 FabIgG format can be used. FIGS. 15D-15G shows exemplary trispecific antibody degraders using the 2+1 FabIgG format. Trispecific antibodies are generated targeting RNF43 (RNF43.37.39), ZNRF3 (ZNRF3-6), and IGF1R (Cixumumab) or EGFR (Cetuximab). Antibody format is a Fab-IgG/IgG with the receptor tyrosine kinase antibody placed at the internal position and ligase antibodies placed at the alternatively at the two external positions. This arrangement may optimize the proximity to each anti-ligase antigen binding domain and further improve the efficiency of degradation The Fab-IgG half-antibody incorporates LC-pairing mutations (Dillon et al. mAbs, 9:2, 213-230, 2017) to allow efficient pairing of the LC arm. The FabIgG and IgG half antibodies are assembled using methods well-known to those in the art. Assessment of these antibodies in the cell surface clearance assay described in Example 5 finds that they clear IGF1R more efficiently than either of the corresponding 1+1 bispecifics in cells with substantial expression of each.

### Example 26: Monitoring IGF1R Surface Clearance after Combinations of Bispecifics

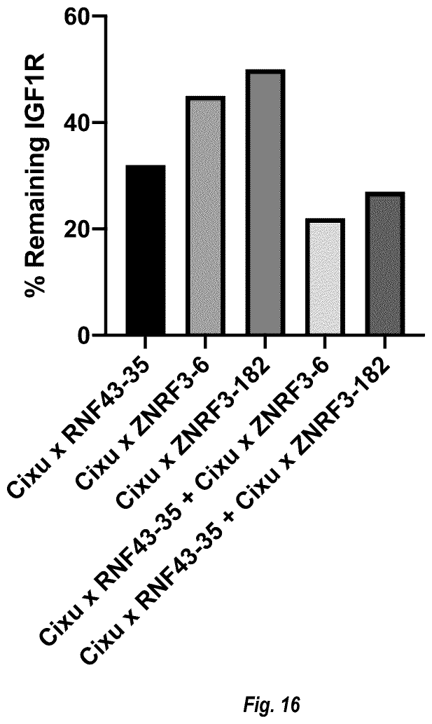

The activity of multiple bispecific antibodies showed saturated at clearance of <100% for HER2, EGFR, and IGF1R. We hypothesized that simultaneously addressing one target with two different ligses might allow us to enhance clearance, due to different rate-limiting steps. To investigate this hypothesis HibiT tagged IGF1R HT29 cells were seeded overnight at 100,000 cells per well in a Corning® 96-well Flat Clear Bottom White Polystyrene TC-treated Microplates and incubated in ATCC recommended media. 1 μg/mL of a single bispecific antibody (targeting IGF1R and ZNRF3 or RNF43) or combination of two bispecific antibodies (the first targeting IGF1R and RNF43, and the second targeting IGF1R and ZNRF3) were added to the cells on the second day and incubated for 24 hours. The luminescence signal of IGF1R-HibiT was detected using the HibiT extracellular system following the description written in the previous example. Percent IGF1R was calculated using the equation % IGF1R=(1−(Untreated samples RLU-treated samples RLU)/Untreated samples RLU)*100. Two different combinations of bispecifics, resulted in clearance of IGF1R that was more efficient than the saturating levels of either individual bispecific (FIG. 16).

### Example 27: Degradation of IGF1R Across Multiple CRC Lines

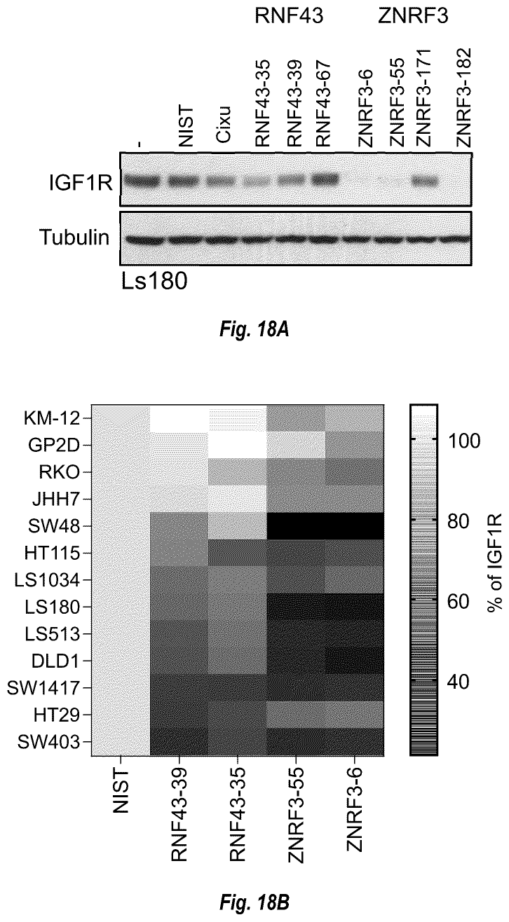

A subset of RNF43 and ZNRF3 antibodies discovered as described above were reformatted into bispecific antibodies targeting the respective ligases and IGF1R (cixutumumab). Activity of these ligases was assessed by their ability to drive whole cell degradation of IGF1R in DLD1 cell and in a large panel of colon cancer cell lines (FIG. 18A, B). Of note, KM-12, RKO, GP2D and JHH7 cells do not display appreciable ligase expression and consequently seve as negative control (FIG. 18B).

### Example 28: Assessment of Target Ubiquitylation Upon Bivalent/Bispecific Antibody Treatment

To evaluate the impact of bispecific antibody treatment on target (IGF1R) ubiquitylation, parental HEK293T cells or HEK293T cells harboring a doxycycline inducible construct of ZNRF3 wild type (WT) or the delta RING mutant (ΔRING) were subjected to a TUBE2 pulldown and samples were assessed by Western blot analysis. In brief, 10 million cells were plated in 10 cm dishes in the presence of doxycycline (1 pig/ml or 31 25 ng/ml) to induce the expression of N-terminally gD and C-terminally FLAG tagged ZNRF3 WT or ZNRF3 ΔRING, respectively. Following 24 hours of doxycycline induction cells were subjected to a media exchange (±doxycycline) and treated with gDxCixutumumab (IGF1R) bispecific antibody (0.5 μg/ml). After two hours of bispecific antibody treatment at 37° C. cells were lysed in 500 pal GST lysis buffer [25 mM Tris·HCl, pH 7.2, 150 mM NaCl, 5 mM MgCl2, 1% NP-40 and 5% glycerol, 1% Halt protease & phosphatase inhibitor cocktail, 50 μM PR-619, 5 mM 1,10 phenanthroline, 10 μM MG132]. Cleared lysates were divided equally and incubated with either 20 μl/sample prewashed Pierce HA epitope tag antibody agarose conjugate (2-2.2.14) (Thermo Scientific; REF 26182) for non-specific pulldown evaluation or 20 μl/sample prewashed agarose-TUBE2 beads (Life Sensors; REF UM402) to enrich polyubiquitinated proteins. Lysate-bead mixtures were incubated for 2 hours and 30 minutes at 4° C. For input samples, 30 μl were used from the prepared pulldown samples prior to incubation. Beads were washed following incubation and prepared for Western blot analysis (FIG. 19B).

Furthermore, IGF1R ubiquitylation following IGF1 stimulation (R&D systems, REF 291-G1), bivalent antibody treatment (CixutununabXCixutumumab), control bispecific antibody treatment (CixutumumabXNIST) or ligase-based bispecific antibody treatment (CixutunutmabXRNF43-35; CixutumurnabXZNRF3-55) was evaluated in HJT29 cells. Briefly, 20 million cells were plated in 15 cm dishes. Cells were subjected to serum starvation 48 hours post plating. Following 22 hours serum starvation, cells were subjected to a media. exchange (serum starved media) concomitant with bivalent or bispecific antibody treatment (1 μg/ml). Cells were incubated for 2 hours and 1 5 minutes at 37° C. For IGF1 stimulation, cells were treated with 50 ng/ml IGF1 5 minutes prior to cell lysis. Following incubation, cells were scarped in 800 μl PBS and cell pellets were resuspended in 1.2 ml GST lysis buffer [25 mM Tris·Cl, pH 7.2, 150 mM NaCl, 5 mM MgCl2, 1% NP-40 and 5% glycerol, 1% Halt protease & phosphatase inhibitor cocktail, 10 mM NEM, 1 mM PMSF]. Cleared lysates were divided equally and incubated with 25 pal/sample prewashed Dynabeads protein G (ThermoFisher Scientific; REF 10004D) conjugated with either rabbit (DA1E) mAb IgG XP isotype control (cell signaling; REF 3900) for non-specific bait protein precipitation or IGF-1 receptor β (D23H3) XP rabbit mAb (cell signaling; REF 9750) for IGF1Rβ immunoprecipitation at 1.87 μg/sample. Lysate-bead mixtures were incubated for 24 hours at 4° C. For input samples, 60 μl were used from the prepared pulldown samples prior to incubation. Beads were washed following incubation and prepared for Western blot analysis (FIG. 19A).

### Example 29: Validation of Ligase Activity Requirements Across Cell Lines and Targets

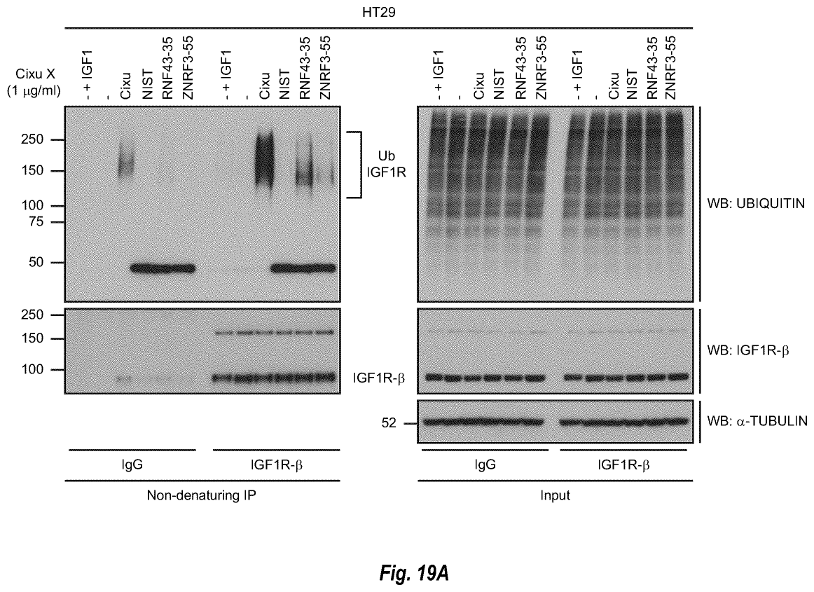

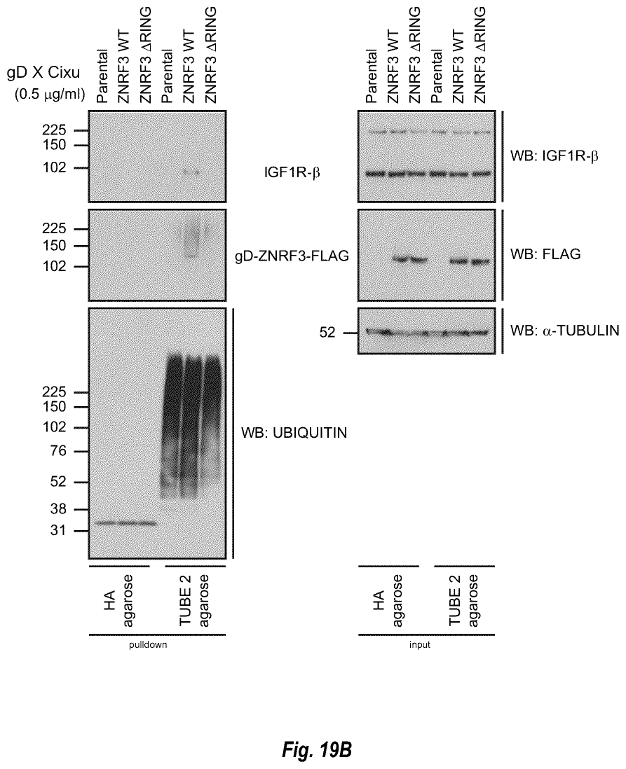

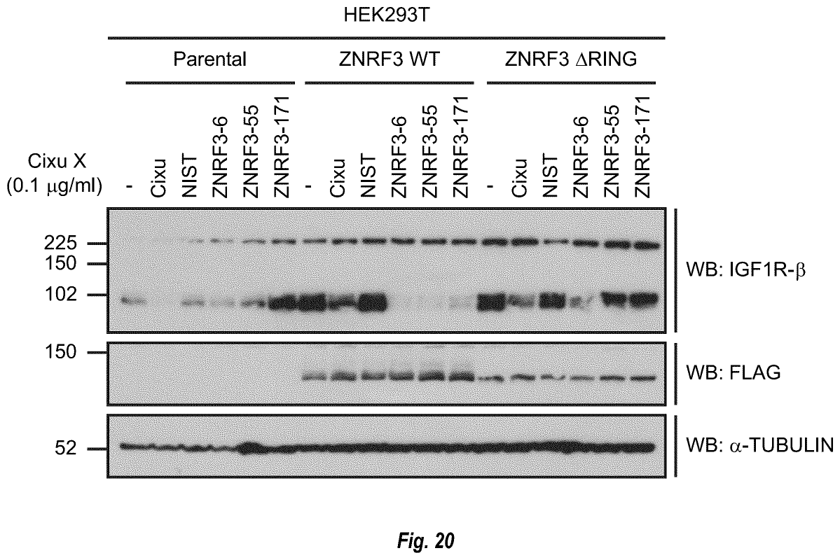

To substantiate the claim described above for HER2 in Example 10, we probed whether the ligase activity ligase was also required for degradation of additional targets. As demonstrated in FIG. 20, deletion of the RING domain from ZNRF3 prevented IGF1R degradation after ligase dimerization after treatment with various ZNRF3/IGF1R bispecific antibodies. Furthermore, we took advantage of naturally occurring RNF43 mutations in a small subset of colon cancers. We selected SW48 as it has a frameshift deletion within the RING domain of RNF43 (FIG. 21A) yet it maintains cell surface ligase expression (FIG. 21B). Dimerization of RNF43/IGF1R bispecific antibodies in that cell line had no impact on IGF1R level, demonstrating that RING and the c-terminus of RNF43 is required for ligase activity. Of note, ZNRF3 was still competent for IGF1R degradation when a ZNRF3/IGF1R antibody was used. To further substantiate this claim, we generated endogenous ligase knock out for both RNF43 or ZNRF3 in HT29 cells (FIG. 22 A). Expectedly, RNF43/IGF1R treatment of RNF43 KO had no impact on IGF1R level while ZNRF3/IGF1R treatment could still degrade the receptor. Importantly, RING knock out did not impact either ligase cell surface expression (FIG. 22A FACS panel), but degradation was also rescued, demonstrating that RING/C-terminus presence is required for full degradative potential of either ligase (FIG. 22 B).

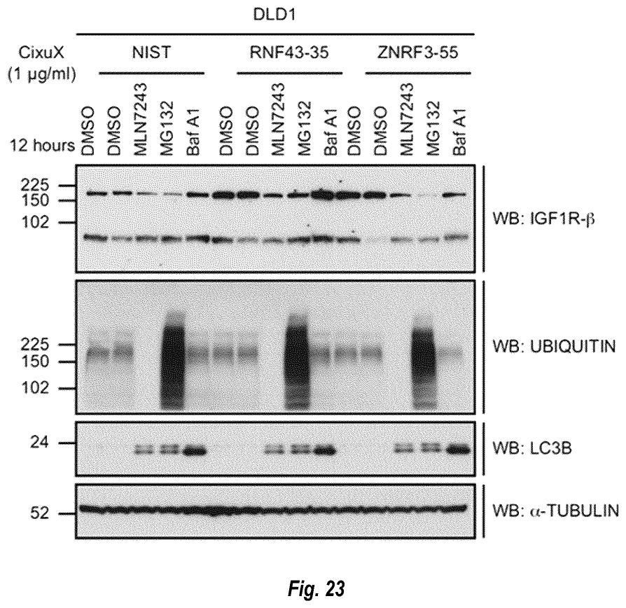

Intestingly, inhibition of the E1 Ubiquitin activating enzyme also rescued degradation, which we found was dependent on both lysosomal and proteasomal activity (FIG. 23) for IGF1R. This is different than what was observed for HER2 where only lysosomal activity was required for degradation of that receptor. Hence, this data suggests that the route of degradation is dictated by the target rather than the ligase itself.

### Example 30: Biological Consequence of Degradation on Cell Signaling and Growth

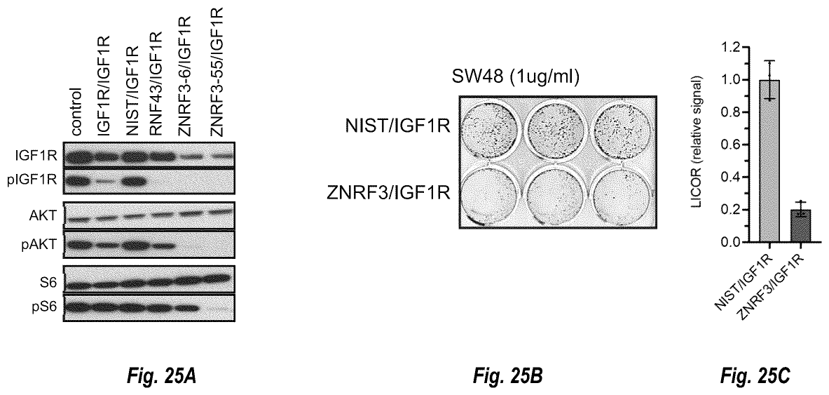

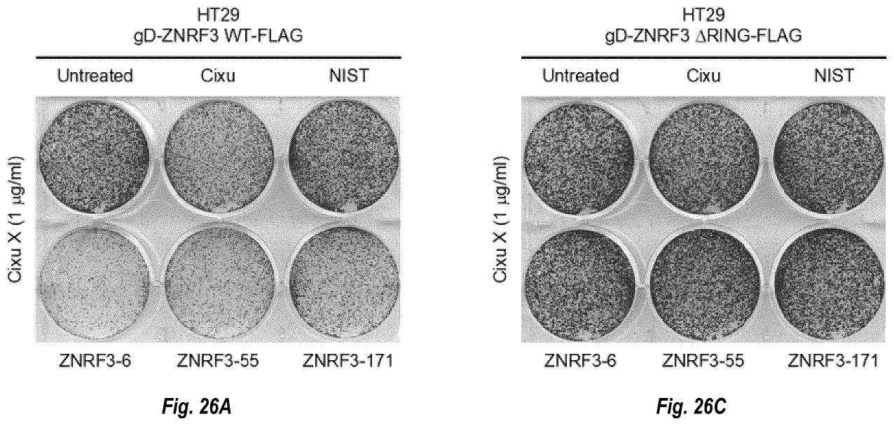

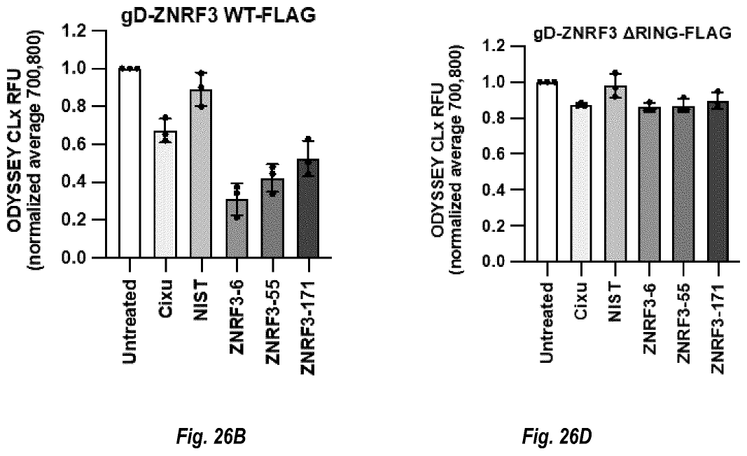

The biological impact of degradation over receptor binding was assessed by looking at modulation of IGF1R signaling. SW48 treated with ZNRF3/IGF1R bispecific antibodies display deep IGF1R degradation. Importantly, this degradation also impacted further downstream signaling as revealed by a complete loss of AKI activity (phosphorylation of AKT) and a decrease phosphorylation of S6, two key downstream effectors of IGF1R (FIG. 25 A). Importantly, the decrease in AKT and S6 activity was superior in ligase treated cells compared to the IGF1R bivalent, demonstrating that degradation provide deeper pathway inhibition than classical blocking antibodies. This decrease in IGF1R signaling also translated functionally as evidenced by a decrease in cell growth in SW48 cells treated with ZNRF3/IGF1R (FIG. 25 B, C). In line with the above, the ligase domain was required to impact cell growth since ZNRF3 RING deficient HT29 failed to impact cell growth while WT ZNRF3 dimerized to IGF1R resulted in slower growth in these cells (FIG. 26A-D).

### Example 31: Endogenous Degradation of Additional Substrate

Bispecific antibodies are generated targeting the PD-I. receptor and exemplary ligase antibodies described above. As depicted in FIG. 27, treatment of SW48 cells with ZNRF3/PDL1 revealed profound and near complete degradation of PD-L1 demonstrating the breadth of degradation across multiple substrates.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### Example 32. In Vivo Assessment

An attractive advantage of the PROTAB technology is that it may overcome the challenges shared by small molecule intracellular degraders, such as limited bioavailability and cell permeability, which can limit their in vivo activity.

Approximately 10⁶ SW48 cells were resuspended in PBS basal media, admixed with 50% Matrigel (Corning) to a final volume of 200 μl, and injected subcutaneously in the left flank of NSG mice (NOD.Cg-PrkdcscidII2rgtm1Wjl/SzJ (NSG) (colony 005557). Mice were purchased from the Jackson Laboratory). Females of 6 to 12 weeks old were used for experiments. Tumor dimensions were measured using calipers and tumor volume was calculated as 0.523×length×width×width. Animals were humanely euthanized according to the following criteria: clinical signs of persistent distress or pain, significant body-weight loss (>20%), tumor size exceeding 2,500 mm³, or when tumors ulcerated. Maximum tumor size permitted by the Institutional Animal Care and Use Committee (IACUC) is 3,000 mm³ and in none of the experiments was this limit exceeded. When tumors reached ˜400 mm³, animals were randomized to receive a single intraperitoneal injection of the PROTABs. All mice were euthanized 72 hr after injection and tumors were collected for further processing.

The lumen implantation procedure has previously been described (de Sousa E Melo F et al. Modeling Colorectal Cancer Progression Through Orthotopic Implantation of Organoids. Methods Mol Biol 2171, 331-346 (2020)). Briefly, mice were anesthetized by isoflurane inhalation and injected intraperitoneally (i.p.) with buprenorphine at 0.05 to 0.1 mg/kg. A blunt-ended hemostat (Micro-Mosquito, No. 13010-12, Fine Science Tools) was inserted ˜1 cm into the anus. The hemostat was angled toward the mucosa and opened slightly such that a single mucosal fold could be clasped by closing the hemostat to the first notch. The hemostat was retracted from the anus, exposing the clasped exteriorized mucosa. A 10 μL of solution containing 50,000 cells admixed with 50% matrigel (Corning) in PBS was directly injected into the colonic mucosae. After reversing the prolapse, the hemostat was then released.

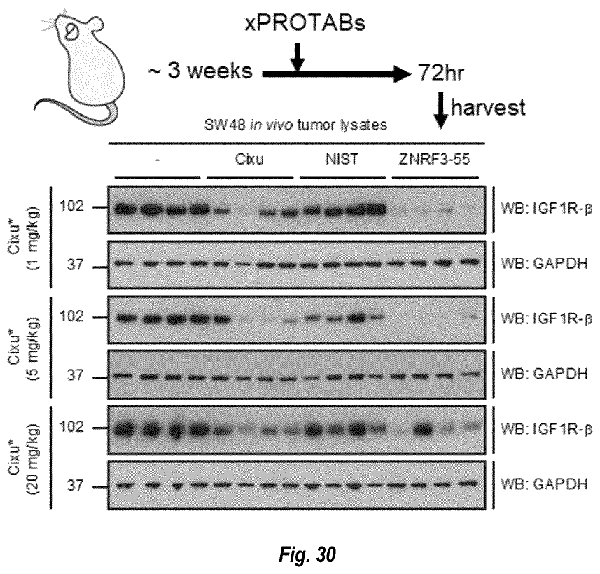

In vivo activity was assessed in SW48 tumor bearing mice (FIG. 30). The upper portion of FIG. 30 shows a schematic representation of the SW48 xenograft model used to analyze the effect of a Cixutuzumab (anti-IGF1R)-based bivalent antibody, NIST* IGF1R (Cixu) bispecific antibody or the indicated ZNRF3*IGF1R (Cixu) bispecific PROTAB in vivo. Following three weeks of SW48 implantation, animals were subjected to antibody treatment. Tumors were collected, homogenized and analyzed biochemically 72 hours post antibody treatment.

The lower portion of FIG. 30 shows Western blot analysis of SW48 in vivo tumor lysates derived from mice left untreated (−) or subjected to a Cixu bivalent antibody, NIST*IGF1R (Cixu) or a ZNRF3-55*IGF1R (Cixu) bispecific PROTAB for 72 hours. Data are representative of four animals per treatment group. Endogenous IGF1Rβ levels were detected. GAPDH was used as a loading control.

The results demonstrate that a single dose of a ZNRF3-based IGF1R PROTABs drove substantial degradation of IGF1R. Intriguingly, IGF1R degradation was partially suppressed at the highest dose (FIG. 30), suggestive of a hook effect that has been reported with some PROTACs (Kannt A et al. Expanding the arsenal of E3 ubiquitin ligases for proximity-induced protein degradation. Cell Chem Biol 28, 1014-1031. (2021); Khan, S et al. PROteolysis TArgeting Chimeras (PROTACs) as emerging anticancer therapeutics. Oncogene 39, 4909-4924 (2020)).

### IV. Sequences

The table below provides exemplary sequences referred to in this disclosure.

| SEQ | | |
|---|---|---|
| ID | | |
| NO | DESCRIPTION | SEQUENCE |
| 1 | gD.gD.mIgG2a. | ATGGGATGGTCATGTATCATCCTTTTTCTAGTAGCAACTGCAACCGGTGTACA |
| | LALAPG.AV- | TTCAGAGGTTCAGCTGGTGGAGTCTGGCGGTGGCCTGGTGCAGCCAGGGGGCT |
| | Hole (heavy | CACTCCGTTTGTCCTGTGCCGCTTCTGGCTACTCCATCACCTCCGACTTTGCC |
| | chain (HC) | TGGAACTGGGTCCGTCAGGCCCCGGGTAAGGGCCTGGAATGGGTTGGATACAT |
| | DNA including | TAGTTACTCTGGAACCACTAGCTATAACCCTAGCCTGAAGTCCCGTATCACTA |
| | signal | TAAGTCGCGACAATTCCAAAAACACATTCTACCTGCAGATGAACAGCCTGCGT |
| | peptide (SP)) | GCTGAGGACACTGCCGTCTATTATTGTGCTCGAGAAAACTACTATGGCCGTTC |
| | | TCACGTTGGGTACTTCGACGTCTGGGGTCAAGGAACCCTGGTCACCGTCTCGA |
| | | GTGCCAAAACAACAGCCCCATCGGTCTATCCACTGGCTCCTGTGTGTGGAGAT |
| | | ACAACTGGCTCCTCGGTGACTCTAGGATGCCTGGTCAAGGGTTATTTCCCTGA |
| | | GCCAGTGACCTTGACCTGGAACTCTGGATCCCTGTCCAGTGGTGTGCACACCT |
| | | TCCCAGCTGTCCTGCAGTCTGACCTCTACACCCTCAGCAGCTCAGTGACTGTA |
| | | ACGTCGAGCACCTGGCCCAGCCAGTCCATCACCTGCAATGTGGCCCACCCGGC |
| | | AAGCAGCACCAAGGTGGACAAGAAAATTGAGCCCAGAGGACCCACAATCAAGC |
| | | CCTGTCCTCCATGCAAATGCCCAGCACCTAACGCCGCGGGTGGACCATCCGTC |
| | | TTCATCTTCCCTCCAAAGATCAAGGATGTACTCATGATCTCCCTGAGCCCCAT |
| | | AGTCACATGTGTGGTGGTGGATGTGAGCGAGGATGACCCAGATGTCCAGATCA |
| | | GCTGGTTTGTGAACAACGTGGAAGTACACACAGCTCAGACACAAACCCATAGA |
| | | GAGGATTACAACAGTACTCTACGCGTGGTCAGTGCCCTCCCCATCCAGCACCA |
| | | GGACTGGATGAGTGGCAAGGAGTTCAAATGCAAGGTCAACAACAAAGACCTCG |
| | | GAGCGCCCATCGAGAGAACCATCTCAAAACCCAAAGGGTCAGTAAGAGCTCCA |
| | | CAGGTATATGTCTTGCCTCCACCAGAAGAAGAGATGACTAAGAAACAGGTCAC |
| | | TCTGACCTGCGCTGTCACAGACTTCATGCCTGAAGACATTTACGTGGAGTGGA |
| | | CCAACAACGGGAAAACAGAGCTAAACTACAAGAACACTGAACCAGTCCTGGAC |
| | | TCTGATGGTTCTTACTTCATGGTTAGCAAGCTGAGAGTGGAAAAGAAGAACTG |
| | | GGTGGAAAGAAATAGCTACTCCTGTTCAGTGGTCCACGAGGGTCTGCACAATC |
| | | ACCACACGACTAAGAGCTTCTCCCGGACTCCGGGTAAATGA |
| 2 | gD.gD.mIgG2a. | ATGGGATGGTCATGTATCATCCTTTTTCTAGTAGCAACTGCAACTGGAGTACA |
| | LALAPG.AV- | TTCAGATATCCAGATGACCCAGTCCCCGAGCTCCCTGTCCGCCTCTGTGGGCG |
| | Hole (light | ATAGGGTCACCATCACCTGCCGTGCCAGTGCGTCTGTCGACAGCTATGGTAAC |
| | chain (LC) | AGCTTCATCCACTGGTATCAGCAGAAACCAGGAAAAGCTCCGAAACTACTGAT |
| | DNA including | TTACCGGGCCTCGGACCTGGAGTCTGGAGTCCCTTCTCGCTTCTCTGGATCCG |
| | SP) | GTTCTGGGACGGATTTCACTCTGACCATCAGCAGTCTGCAGCCAGAAGACTTC |
| | | GCAACTTATTACTGTCAGCAAAATTACGCGGATCCGTTCACATTTGGACAGGG |
| | | TACCAAGGTGGAGATCAAGCGCGCTGATGCTGCACCAACTGTATCCATCTTCC |
| | | CACCATCCAGTGAGCAGTTAACATCTGGAGGTGCCTCAGTCGTGTGCTTCTTG |
| | | AACAACTTCTACCCCAAAGACATCAATGTCAAGTGGAAGATTGATGGCAGTGA |
| | | ACGACAAAATGGCGTCCTGAACAGTTGGACTGATCAGGACAGCAAAGACAGCA |
| | | CCTACAGCATGAGCAGCACCCTCACGTTGACCAAGGACGAGTATGAACGACAT |
| | | AACAGCTATACCTGTGAGGCCACTCACAAGACATCAACTTCACCCATTGTCAA |
| | | GAGCTTCAACAGGAATGAGTGTTGA |
| 3 | gD.gD.mIgG2a. | EVQLVESGGG LVQPGGSLRL SCAASGYSIT SDFAWNWVRQ |
| | LALAPG.AV- | APGKGLEWVG YISYSGTTSY NPSLKSRITI SRDNSKNTFY |
| | Hole heavy | LQMNSLRAED TAVYYCAREN YYGRSHVGYF DVWGQGTLVT |
| | chain (mature | VSSAKTTAPS VYPLAPVCGD TTGSSVTLGC LVKGYFPEPV |
| | sequence) | TLTWNSGSLS SGVHTFPAVL QSDLYTLSSS VTVTSSTWPS |
| | | QSITCNVAHP ASSTKVDKKI EPRGPTIKPC PPCKCPAPNA |
| | | AGGPSVFIFP PKIKDVLMIS LSPIVTCVVV DVSEDDPDVQ |
| | | ISWFVNNVEV HTAQTQTHRE DYNSTLRVVS ALPIQHQDWM |
| | | SGKEFKCKVN NKDLGAPIER TISKPKGSVR APQVYVLPPP |
| | | EEEMTKKQVT LTCAVTDEMP EDIYVEWINN GKTELNYKNT |
| | | EPVLDSDGSY FMVSKLRVEK KNWVERNSYS CSVVHEGLHN |
| | | HHTTKSFSRT PGK |
| 4 | gD.gD.mIgG2a. | DIQMTQSPSS LSASVGDRVT ITCRASASVD SYGNSFIHWY |
| | LALAPG.AV- | QQKPGKAPKL LIYRASDLES GVPSRESGSG SGTDFTLTIS |
| | Hole light | SLQPEDFATY YCQQNYADPF TEGQGTKVEI KRADAAPTVS |
| | chain (mature | IFPPSSEQLT SGGASVVCFL NNFYPKDINV KWKIDGSERQ |
| | sequence) | NGVLNSWTDQ DSKDSTYSMS STLTLTKDEY ERHNSYTCEA |
| | | THKTSTSPIV KSENRNEC |
| 5 | xRNF43.hSC37. | ATGGGATGGTCATGTATCATCCTTTTTCTAGTAGCAACTGCAACCGGTGTACA |
| | 39.mIgG2a.LAL | TTCACAGGTGCAGCTGGTGCAGTCCGGCGCCGAGGTGAAGAAGCCCGGCGCCT |
| | APG.AV- | CCGTGAAGGTGTCCTGCAAGGCCTCCGGCTACACCTTCACCACCTACACCATC |
| | Hole.AX (HC | CACTGGGTGAGGCAGGCCCCCGGCCAGGGCCTGGAGTGGATGGGCTACATCAA |
| | DNA with SP) | CCCCAGGTCCGGCTACACCGAGTACAACCAGAAGTTCCAGGACAGGGTGACCA |
| | | TGACCAGGGACACCTCCACCTCCACCGTGTACATGGAGCTGTCCTCCCTGAGG |
| | | TCCGAGGACACCGCCGTGTACTACTGCGCCAGGTCCTACGAGTTCTGGGGCCA |
| | | GGGCACCACCGTGACCGTCTCGAGTGCCAAAACAACAGCCCCATCGGTCTATC |
| | | CACTGGCTCCTGTGTGTGGAGATACAACTGGCTCCTCGGTGACTCTAGGATGC |
| | | CTGGTCAAGGGTTATTTCCCTGAGCCAGTGACCTTGACCTGGAACTCTGGATC |
| | | CCTGTCCAGTGGTGTGCACACCTTCCCAGCTGTCCTGCAGTCTGACCTCTACA |
| | | CCCTCAGCAGCTCAGTGACTGTAACGTCGAGCACCTGGCCCAGCCAGTCCATC |
| | | ACCTGCAATGTGGCCCACCCGGCAAGCAGCACCAAGGTGGACAAGAAAATTGA |
| | | GCCCAGAGGACCCACAATCAAGCCCTGTCCTCCATGCAAATGCCCAGCACCTA |
| | | ACGCCGCGGGTGGACCATCCGTCTTCATCTTCCCTCCAAAGATCAAGGATGTA |
| | | CTCATGATCTCCCTGAGCCCCATAGTCACATGTGTGGTGGTGGATGTGAGCGA |
| | | GGATGACCCAGATGTCCAGATCAGCTGGTTTGTGAACAACGTGGAAGTACACA |
| | | CAGCTCAGACACAAACCCATAGAGAGGATTACAACAGTACTCTACGCGTGGTC |
| | | AGTGCCCTCCCCATCCAGCACCAGGACTGGATGAGTGGCAAGGAGTTCAAATG |
| | | CAAGGTCAACAACAAAGACCTCGGAGCGCCCATCGAGAGAACCATCTCAAAAC |
| | | CCAAAGGGTCAGTAAGAGCTCCACAGGTATATGTCTTGCCTCCACCAGAAGAA |
| | | GAGATGACTAAGAAACAGGTCACTCTGACCTGCGCTGTCACAGACTTCATGCC |
| | | TGAAGACATTTACGTGGAGTGGACCAACAACGGGAAAACAGAGCTAAACTACA |
| | | AGAACACTGAACCAGTCCTGGACTCTGATGGTTCTTACTTCATGGTTAGCAAG |
| | | CTGAGAGTGGAAAAGAAGAACTGGGTGGAAAGAAATAGCTACTCCTGTTCAGT |
| | | GGTCCACGAGGGTCTGCACAATCACCACACGACTAAGAGCTTCTCCCGGACTC |
| | | CGGGTAAATGA |
| 6 | xRNF43.hSC37. | ATGGGATGGTCATGTATCATCCTTTTTCTAGTAGCAACTGCAACCGGTGTACA |
| | 39.mIgG2a.LAL | TTCAGAGATCGTGATGACCCAGTCCCCCGCCACCCTGTCCGTGTCCCCCGGCG |
| | APG.AV- | AGAGGGCCACCCTGTCCTGCAAGGCCTCCCAGAACGTGGGCATCAACGTGGCC |
| | Hole.AX (LC | TGGTATCAGCAGAAGCCCGGCCAGGCCCCCAGGGCCCTGATCTACTCCGCCTC |
| | DNA with SP) | CTACAGGTACTCCGGCATCCCCGCCAGGTTCTCCGGCTCCGGCTCCGGCACCG |
| | | AGTTCACCCTGACCATCTCCTCCCTGCAGTCCGAGGACTTCGCCGTGTACTAC |
| | | TGCCACCAGTACAAGACCTACCCCTACACCTTCGGCGGCGGTACCAAGCTGGA |
| | | GATCAAGCGCGCTGATGCTGCACCAACTGTATCCATCTTCCCACCATCCAGTG |
| | | AGCAGTTAACATCTGGAGGTGCCTCAGTCGTGTGCTTCTTGAACAACTTCTAC |
| | | CCCAAAGACATCAATGTCAAGTGGAAGATTGATGGCAGTGAACGACAAAATGG |
| | | CGTCCTGAACAGTTGGACTGATCAGGACAGCAAAGACAGCACCTACAGCATGA |
| | | GCAGCACCCTCACGTTGACCAAGGACGAGTATGAACGACATAACAGCTATACC |
| | | TGTGAGGCCACTCACAAGACATCAACTTCACCCATTGTCAAGAGCTTCAACAG |
| | | GAATGAGTGTTG |
| 7 | XRNF43.hSC37. | EVQLVQSGAE VKKPGASVKV SCKASGYTFT TYTIHWVRQA |
| | 39.mIgG2a.LAL | PGQGLEWMGY INPRSGYTEY NQKFQDRVTM TRDTSTSTVY |
| | APG.AV- | MELSSLRSED TAVYYCARSY EFWGQGTTVT VSSAKTTAPS |
| | Hole.AX | VYPLAPVCGD TTGSSVTLGC LVKGYFPEPV TLTWNSGSLS |
| | mature HC | SGVHTFPAVL QSDLYTLSSS VTVTSSTWPS QSITCNVAHP |
| | | ASSTKVDKKI EPRGPTIKPC PPCKCPAPNA AGGPSVFIFP |
| | | PKIKDVLMIS LSPIVTCVVV DVSEDDPDVQ ISWFVNNVEV |
| | | HTAQTQTHRE DYNSTLRVVS ALPIQHQDWM SGKEFKCKVN |
| | | NKDLGAPIER TISKPKGSVR APQVYVLPPP EEEMTKKQVT |
| | | LTCAVTDEMP EDIYVEWTNN GKTELNYKNT EPVLDSDGSY |
| | | FMVSKLRVEK KNWVERNSYS CSVVHEGLHN HHTTKSFSRT PGK |
| 8 | xRNF43.hSC37. | EIVMTQSPAT LSVSPGERAT LSCKASQNVG INVAWYQQKP |
| | 39.mIgG2a.LAL | GQAPRALIYS ASYRYSGIPA RFSGSGSGTE FTLTISSLQS |
| | APG.AV- | EDFAVYYCHQ YKTYPYTFGG GTKLEIKRAD AAPTVSIFPP |
| | Hole.AX | SSEQLTSGGA SVVCFLNNFY PKDINVKWKI DGSERQNGVL |
| | mature LC | NSWTDQDSKD STYSMSSTLT LTKDEYERHN SYTCEATHKT |
| | | STSPIVKSEN RNEC |
| 9 | NIST.mIgG2a.L | ATGGGATGGTCATGTATCATCCTTTTTCTAGTAGCAACTGCAACCGGTGTACA |
| | ALAPG.AV-Hole | TTCACAGGTGACCCTGAGGGAGTCCGGCCCCGCCCTGGTGAAGCCCACCCAGA |
| | (HC DNA with | CCCTGACCCTGACCTGCACCTTCTCCGGCTTCTCCCTGTCCACCGCCGGCATG |
| | SP) | TCCGTGGGCTGGATCAGGCAGCCCCCCGGCAAGGCCCTGGAGTGGCTGGCCGA |
| | | CATCTGGTGGGACGACAAGAAGCACTACAACCCCTCCCTGAAGGACAGGCTGA |
| | | CCATCTCCAAGGACACCTCCAAGAACCAGGTGGTGCTGAAGGTGACCAACATG |
| | | GACCCCGCCGACACCGCCACCTACTACTGCGCCAGGGACATGATCTTCAACTT |
| | | CTACTTCGACGTGTGGGGCCAGGGCACCACCGTGACCGTCTCGAGTGCCAAAA |
| | | CAACAGCCCCATCGGTCTATCCACTGGCTCCTGTGTGTGGAGATACAACTGGC |
| | | TCCTCGGTGACTCTAGGATGCCTGGTCAAGGGTTATTTCCCTGAGCCAGTGAC |
| | | CTTGACCTGGAACTCTGGATCCCTGTCCAGTGGTGTGCACACCTTCCCAGCTG |
| | | TCCTGCAGTCTGACCTCT |
| | | ACACCCTCAGCAGCTCAGTGACTGTAACGTCGAGCACCTGGCCCAGCCAGTCC |
| | | ATCACCTGCAATGTGGCCCACCCGGCAAGCAGCACCAAGGTGGACAAGAAAAT |
| | | TGAGCCCAGAGGACCCACAATCAAGCCCTGTCCTCCATGCAAATGCCCAGCAC |
| | | CTAACGCCGCGGGTGGACCATCCGTCTTCATCTTCCCTCCAAAGATCAAGGAT |
| | | GTACTCATGATCTCCCTGAGCCCCATAGTCACATGTGTGGTGGTGGATGTGAG |
| | | CGAGGATGACCCAGATGTCCAGATCAGCTGGTTTGTGAACAACGTGGAAGTAC |
| | | ACACAGCTCAGACACAAACCCATAGAGAGGATTACAACAGTACTCTACGCGTG |
| | | GTCAGTGCCCTCCCCATCCAGCACCAGGACTGGATGAGTGGCAAGGAGTTCAA |
| | | ATGCAAGGTCAACAACAAAGACCTCGGAGCGCCCATCGAGAGAACCATCTCAA |
| | | AACCCAAAGGGTCAGTAAGAGCTCCACAGGTATATGTCTTGCCTCCACCAGAA |
| | | GAAGAGATGACTAAGAAACAGGTCACTCTGACCTGCGCTGTCACAGACTTCAT |
| | | GCCTGAAGACATTTACGTGGAGTGGACCAACAACGGGAAAACAGAGCTAAACT |
| | | ACAAGAACACTGAACCAGTCCTGGACTCTGATGGTTCTTACTTCATGGTTAGC |
| | | AAGCTGAGAGTGGAAAAGAAGAACTGGGTGGAAAGAAATAGCTACTCCTGTTC |
| | | AGTGGTCCACGAGGGTCTGCACAATCACCACACGACTAAGAGCTTCTCCCGGA |
| | | CTCCGGGTAAATGA |
| 10 | NIST.mIgG2a.L | ATGGGATGGTCATGTATCATCCTTTTTCTAGTAGCAACTGCAACCGGTGTACA |
| | ALAPG.AV-Hole | TTCAGACATCCAGATGACCCAGTCCCCCTCCACCCTGTCCGCCTCCGTGGGCG |
| | (LC DNA with | ACAGGGTGACCATCACCTGCTCCGCCTCCTCCAGGGTGGGCTACATGCACTGG |
| | SP) | TATCAGCAGAAGCCCGGCAAGGCCCCCAAGCTGCTGATCTACGACACCTCCAA |
| | | GCTGGCCTCCGGCGTGCCCTCCAGGTTCTCCGGCTCCGGCTCCGGCACCGAGT |
| | | TCACCCTGACCATCTCCTCCCTGCAGCCCGACGACTTCGCCACCTACTACTGC |
| | | TTCCAGGGCTCCGGCTACCCCTTCACCTTCGGCGGCGGTACCAAGGTGGAGAT |
| | | CAAGCGCGCTGATGCTGCACCAACTGTATCCATCTTCCCACCATCCAGTGAGC |
| | | AGTTAACATCTGGAGGTGCCTCAGTCGTGTGCTTCTTGAACAACTTCTACCCC |
| | | AAAGACATCAATGTCAAGTGGAAGATTGATGGCAGTGAACGACAAAATGGCGT |
| | | CCTGAACAGTTGGACTGATCAGGACAGCAAAGACAGCACCTACAGCATGAGCA |
| | | GCACCCTCACGTTGACCAAGGACGAGTATGAACGACATAACAGCTATACCTGT |
| | | GAGGCCACTCACAAGACATCAACTTCACCCATTGTCAAGAGCTTCAACAGGAA |
| | | TGAGTGTTGA |
| 11 | NIST.mIgG2a.L | QVTLRESGPA LVKPTQTLTL TCTFSGESLS TAGMSVGWIR |
| | ALAPG.AV-Hole | QPPGKALEWL ADIWWDDKKH YNPSLKDRLT ISKDTSKNQV |
| | (HC mature) | VLKVTNMDPA DTATYYCARD MIFNFYFDVW GQGTTVTVSS |
| | | AKTTAPSVYP LAPVCGDTTG SSVTLGCLVK GYFPEPVTLT |
| | | WNSGSLSSGV HTFPAVLQSD LYTLSSSVTV TSSTWPSQSI |
| | | TCNVAHPASS TKVDKKIEPR GPTIKPCPPC KCPAPNAAGG |
| | | PSVFIFPPKI KDVLMISLSP IVTCVVVDVS EDDPDVQISW |
| | | FVNNVEVHTA QTQTHREDYN STLRVVSALP IQHQDWMSGK |
| | | EFKCKVNNKD LGAPIERTIS KPKGSVRAPQ VYVLPPPEEE |
| | | MTKKQVTLTC AVTDEMPEDI YVEWTNNGKT ELNYKNTEPV |
| | | LDSDGSYFMV SKLRVEKKNW VERNSYSCSV VHEGLHNHHT TKSFSRTPG |
| 12 | NIST.mIgG2a.L | DIQMTQSPST LSASVGDRVT ITCSASSRVG YMHWYQQKPG |
| | ALAPG.AV-Hole | KAPKLLIYDT SKLASGVPSR FSGSGSGTEF TLTISSLQPD |
| | (LC mature) | DFATYYCFQG SGYPFTFGGG TKVEIKRADA APTVSIFPPS |
| | | SEQLTSGGAS VVCFLNNFYP KDINVKWKID GSERQNGVLN |
| | | SWTDQDSKDS TYSMSSTLTL TKDEYERHNS YTCEATHKTS |
| | | TSPIVKSENR NEC |
| 13 | HER2.4D5VH- | ATGGGATGGTCATGTATCATCCTTTTTCTAGTAGCAACTGCAACCGGTGTACA |
| | mIgG2aCH1- | TTCAGAAGTTCAGCTGGTGGAGTCTGGCGGTGGCCTGGTGCAGCCAGGGGGCT |
| | 4D5VH.mIgG2a. | CACTCCGTTTGTCCTGTGCAGCTTCTGGCTTCAACATTAAAGACACCTATATA |
| | LALAPG.Knob | CACTGGGTGCGTCAGGCCCCGGGTAAGGGCCTGGAATGGGTTGCAAGGATTTA |
| | (HC DNA with | TCCTACGAATGGTTATACTAGATATGCCGATAGCGTCAAGGGCCGTTTCACTA |
| | SP) | TAAGCGCAGACACATCCAAAAACACAGCCTACCTGCAGATGAACAGCCTGCGT |
| | | GCTGAGGACACTGCCGTCTATTATTGTTCTAGATGGGGAGGGGACGGCTTCTA |
| | | TGCTATGGACTACTGGGGTCAAGGAACCCTGGTCACCGTCTCGAGTGCCAAGA |
| | | CCACCGCCCCCAGCGTGTACCCCCTGGCCCCCGTGTGCGGCGACACCACCGGC |
| | | AGCAGCGTGACCCTGGGCTGCCTGGTGAAGGGCTACTTCCCCGAGCCCGTGAC |
| | | CCTGACCTGGAACAGCGGCAGCCTGAGCAGCGGCGTGCACACCTTCCCCGCCG |
| | | TGCTGCAGAGCGACCTGTACACCCTGAGCAGCAGCGTGACCGTGACCAGCAGC |
| | | ACCTGGCCCAGCCAGAGCATCACCTGCAACGTGGCCCACCCCGCCAGCAGCAC |
| | | CAAGGTGGACAAGAAGATCGAGCCCAGAGGCCCCACCATCAAGCCCGAGGTGC |
| | | AGCTGGTGGAGAGCGGCGGCGGCCTGGTGCAGCCCGGCGGCAGCCTGAGACTG |
| | | AGCTGCGCCGCCAGCGGCTTCAACATCAAGGACACCTACATCCACTGGGTGAG |
| | | ACAGGCCCCCGGCAAGGGCCTGGAGTGGGTGGCCAGAATCTACCCCACCAACG |
| | | GCTACACCAGATACGCCGACAGCGTGAAGGGCAGATTCACCATCAGCGCCGAC |
| | | ACCAGCAAGAACACCGCCTACCTGCAGATGAACAGCCTGAGAGCCGAGGACAC |
| | | CGCCGTGTACTACTGCAGCAGATGGGGCGGCGACGGCTTCTACGCCATGGACT |
| | | ACTGGGGCCAGGGCACCCTGGTGACCGTCTCGAGTGCCAAAACAACAGCCCCA |
| | | TCGGTCTATCCACTGGCTCCTGTGTGTGGAGATACAACTGGCTCCTCGGTGAC |
| | | TCTAGGATGCCTGGTCAAGGGTTATTTCCCTGAGCCAGTGACCTTGACCTGGA |
| | | ACTCTGGATCCCTGTCCAGTGGTGTGCACACCTTCCCAGCTGTCCTGCAGTCT |
| | | GACCTCTACACCCTCAGCAGCTCAGTGACTGTAACGTCGAGCACCTGGCCCAG |
| | | CCAGTCCATCACCTGCAATGTGGCCCACCCGGCAAGCAGCACCAAGGTGGACA |
| | | AGAAAATTGAGCCCAGAGGACCCACAATCAAGCCCTGTCCTCCATGCAAATGC |
| | | CCAGCACCTAACGCCGCGGGTGGACCATCCGTCTTCATCTTCCCTCCAAAGAT |
| | | CAAGGATGTACTCATGATCTCCCTGAGCCCCATAGTCACATGTGTGGTGGTGG |
| | | ATGTGAGCGAGGATGACCCAGATGTCCAGATCAGCTGGTTTGTGAACAACGTG |
| | | GAAGTACACACAGCTCAGACACAAACCCATAGAGAGGATTACAACAGTACTCT |
| | | ACGCGTGGTCAGTGCCCTCCCCATCCAGCACCAGGACTGGATGAGTGGCAAGG |
| | | AGTTCAAATGCAAGGTCAACAACAAAGACCTCGGAGCGCCCATCGAGAGAACC |
| | | ATCTCAAAACCCAAAGGGTCAGTAAGAGCTCCACAGGTATATGTCTTGCCTCC |
| | | ACCAGAAGAAGAGATGACTAAGAAACAGGTCACTCTGTGGTGCATGGTCACAG |
| | | ACTTCATGCCTGAAGACATTTACGTGGAGTGGACCAACAACGGGAAAACAGAG |
| | | CTAAACTACAAGAACACTGAACCAGTCCTGGACTCTGATGGTTCTTACTTCAT |
| | | GTACAGCAAGCTGAGAGTGGAAAAGAAGAACTGGGTGGAAAGAAATAGCTACT |
| | | CCTGTTCAGTGGTCCACGAGGGTCTGCACAATCACCACACGACTAAGAGCTTC |
| | | TCCCGGACTCCGGGTAAATGA |
| 14 | HER2.4D5VH- | ATGGGATGGTCATGTATCATCCTTTTTCTAGTAGCAACTGCAACCGGTGTACA |
| | mIgG2aCH1- | TTCAGATATCCAGATGACCCAGTCCCCGAGCTCCCTGTCCGCCTCTGTGGGCG |
| | 4D5VH.mIgG2a. | ATAGGGTCACCATCACCTGCCGTGCCAGTCAGGATGTGAATACTGCTGTAGCC |
| | LALAPG.Knob | TGGTATCAACAGAAACCAGGAAAAGCTCCGAAACTACTGATTTACTCGGCATC |
| | (LC DNA with | CTTCCTCTACTCTGGAGTCCCTTCTCGCTTCTCTGGATCCAGATCTGGGACGG |
| | SP) | ATTTCACTCTGACCATCAGCAGTCTGCAGCCGGAAGACTTCGCAACTTATTAC |
| | | TGTCAGCAACATTATACTACTCCTCCCACGTTCGGACAGGGTACCAAGCTGGA |
| | | GATCAAGCGCGCTGATGCTGCACCAACTGTATCCATCTTCCCACCATCCAGTG |
| | | AGCAGTTAACATCTGGAGGTGCCTCAGTCGTGTGCTTCTTGAACAACTTCTAC |
| | | CCCAAAGACATCAATGTCAAGTGGAAGATTGATGGCAGTGAACGACAAAATGG |
| | | CGTCCTGAACAGTTGGACTGATCAGGACAGCAAAGACAGCACCTACAGCATGA |
| | | GCAGCACCCTCACGTTGACCAAGGACGAGTATGAACGACATAACAGCTATACC |
| | | TGTGAGGCCACTCACAAGACATCAACTTCACCCATTGTCAAGAGCTTCAACAG |
| | | GAATGAGTGTTGA |
| 15 | HER2.4D5VH- | EVQLVESGGG LVQPGGSLRL SCAASGFNIK DTYIHWVRQA |
| | mIgG2aCH1- | PGKGLEWVAR IYPTNGYTRY ADSVKGRFTI SADTSKNTAY |
| | 4D5VH.mIgG2a. | LQMNSLRAED TAVYYCSRWG GDGFYAMDYW GQGTLVTVSS |
| | LALAPG.Knob | AKTTAPSVYP LAPVCGDTTG SSVTLGCLVK GYFPEPVTLT |
| | (HC mature) | WNSGSLSSGV HTFPAVLQSD LYTLSSSVTV TSSTWPSQSI |
| | | TCNVAHPASS TKVDKKIEPR GPTIKPEVQL VESGGGLVQP |
| | | GGSLRLSCAA SGFNIKDTYI HWVRQAPGKG LEWVARIYPT |
| | | NGYTRYADSV KGRFTISADT SKNTAYLQMN SLRAEDTAVY |
| | | YCSRWGGDGF YAMDYWGQGT LVTVSSAKTT APSVYPLAPV |
| | | CGDTTGSSVT LGCLVKGYFP EPVTLTWNSG SLSSGVHTEP |
| | | AVLQSDLYTL SSSVTVTSST WPSQSITCNV AHPASSTKVD |
| | | KKIEPRGPTI KPCPPCKCPA PNAAGGPSVF IFPPKIKDVL |
| | | MISLSPIVTC VVVDVSEDDP DVQISWFVNN VEVHTAQTQT |
| | | HREDYNSTLR VVSALPIQHQ DWMSGKEFKC KVNNKDLGAP |
| | | IERTISKPKG SVRAPQVYVL PPPEEEMTKK QVTLWCMVTD |
| | | FMPEDIYVEW TNNGKTELNY KNTEPVLDSD GSYFMYSKLR |
| | | VEKKNWVERN SYSCSVVHEG LHNHHTTKSF SRTPGK |
| 16 | HER2.4D5VH- | DIQMTQSPSS LSASVGDRVT ITCRASQDVN TAVAWYQQKP |
| | mIgG2aCH1- | GKAPKLLIYS ASFLYSGVPS RESGSRSGTD FTLTISSLQP |
| | 4 D5VH.mIgG2a. | EDFATYYCQQ HYTTPPTFGQ GTKLEIKRAD AAPTVSIFPP |
| | LALAPG.Knob | SSEQLTSGGA SVVCFLNNFY PKDINVKWKI DGSERQNGVL |
| | (LC mature) | NSWTDQDSKD STYSMSSTLT LTKDEYERHN SYTCEATHKT |
| | | STSPIVKSEN RNEC |
| 17 | HER2.2C4VH- | ATGGGATGGTCATGTATCATCCTTTTTCTAGTAGCAACTGCAACCGGTGTACA |
| | mIgG2aCH1- | TTCAGAGGTGCAGCTGGTGGAGAGCGGCGGCGGCCTGGTGCAGCCAGGCGGCA |
| | 2C4VH.mIgG2a. | GCCTGCGCCTGAGCTGCGCCGCCAGCGGCTTCACCTTCACCGACTACACCATG |
| | LALAPG.Knob | GACTGGGTGCGCCAGGCCCCAGGCAAGGGCCTGGAGTGGGTGGCCGACGTGAA |
| | (HC DNA with | CCCAAACAGCGGCGGCAGCATCTACAACCAGCGCTTCAAGGGCCGCTTCACCC |
| | SP) | TGAGCGTGGACCGCAGCAAGAACACCCTGTACCTGCAGATGAACAGCCTGCGC |
| | | GCCGAGGACACCGCCGTGTACTACTGCGCCCGCAACCTGGGCCCAAGCTTCTA |
| | | CTTCGACTACTGGGGCCAGGGCACCCTGGTGACCGTCTCGAGTGCCAAGACCA |
| | | CCGCCCCCAGCGTGTACCCCCTGGCCCCCGTGTGCGGCGACACCACCGGCAGC |
| | | AGCGTGACCCTGGGCTGCCTGGTGAAGGGCTACTTCCCCGAGCCCGTGACCCT |
| | | GACCTGGAACAGCGGCAGCCTGAGCAGCGGCGTGCACACCTTCCCCGCCGTGC |
| | | TGCAGAGCGACCTGTACACCCTGAGCAGCAGCGTGACCGTGACCAGCAGCACC |
| | | TGGCCCAGCCAGAGCATCACCTGCAACGTGGCCCACCCCGCCAGCAGCACCAA |
| | | GGTGGACAAGAAGATCGAGCCCAGAGGCCCCACCATCAAGCCCGAGGTGCAGC |
| | | TGGTGGAGAGCGGCGGCGGCCTGGTGCAGCCCGGCGGCAGCCTGAGACTGAGC |
| | | TGCGCCGCCAGCGGCTTCACCTTCACCGACTACACCATGGACTGGGTGAGACA |
| | | GGCCCCCGGCAAGGGCCTGGAGTGGGTGGCCGACGTGAACCCCAACAGCGGCG |
| | | GCAGCATCTACAACCAGAGATTCAAGGGCAGATTCACCCTGAGCGTGGACAGA |
| | | AGCAAGAACACCCTGTACCTGCAGATGAACAGCCTGAGAGCCGAGGACACCGC |
| | | CGTGTACTACTGCGCCAGAAACCTGGGCCCCAGCTTCTACTTCGACTACTGGG |
| | | GCCAGGGCACCCTGGTGACCGTCTCGAGTGCCAAAACAACAGCCCCATCGGTC |
| | | TATCCACTGGCTCCTGTGTGTGGAGATACAACTGGCTCCTCGGTGACTCTAGG |
| | | ATGCCTGGTCAAGGGTTATTTCCCTGAGCCAGTGACCTTGACCTGGAACTCTG |
| | | GATCCCTGTCCAGTGGTGTGCACACCTTCCCAGCTGTCCTGCAGTCTGACCTC |
| | | TACACCCTCAGCAGCTCAGTGACTGTAACGTCGAGCACCTGGCCCAGCCAGTC |
| | | CATCACCTGCAATGTGGCCCACCCGGCAAGCAGCACCAAGGTGGACAAGAAAA |
| | | TTGAGCCCAGAGGACCCACAATCAAGCCCTGTCCTCCATGCAAATGCCCAGCA |
| | | CCTAACGCCGCGGGTGGACCATCCGTCTTCATCTTCCCTCCAAAGATCAAGGA |
| | | TGTACTCATGATCTCCCTGAGCCCCATAGTCACATGTGTGGTGGTGGATGTGA |
| | | GCGAGGATGACCCAGATGTCCAGATCAGCTGGTTTGTGAACAACGTGGAAGTA |
| | | CACACAGCTCAGACACAAACCCATAGAGAGGATTACAACAGTACTCTACGCGT |
| | | GGTCAGTGCCCTCCCCATCCAGCACCAGGACTGGATGAGTGGCAAGGAGTTCA |
| | | AATGCAAGGTCAACAACAAAGACCTCGGAGCGCCCATCGAGAGAACCATCTCA |
| | | AAACCCAAAGGGTCAGTAAGAGCTCCACAGGTATATGTCTTGCCTCCACCAGA |
| | | AGAAGAGATGACTAAGAAACAGGTCACTCTGTGGTGCATGGTCACAGACTTCA |
| | | TGCCTGAAGACATTTACGTGGAGTGGACCAACAACGGGAAAACAGAGCTAAAC |
| | | TACAAGAACACTGAACCAGTCCTGGACTCTGATGGTTCTTACTTCATGTACAG |
| | | CAAGCTGAGAGTGGAAAAGAAGAACTGGGTGGAAAGAAATAGCTACTCCTGTT |
| | | CAGTGGTCCACGAGGGTCTGCACAATCACCACACGACTAAGAGCTTCTCCCGG |
| | | ACTCCGGGTAAATGA |
| 18 | HER2.2C4VH- | ATGGGATGGTCATGTATCATCCTTTTTCTAGTAGCAACTGCAACCGGTGTACA |
| | mIgG2aCH1- | TTCAGACATCCAGATGACCCAGAGCCCAAGCAGCCTGAGCGCCAGCGTGGGCG |
| | 2C4VH.mIgG2a. | ACCGCGTGACCATCACCTGCAAGGCCAGCCAGGACGTGAGCATCGGCGTGGCC |
| | LALAPG.Knob | TGGTATCAGCAGAAGCCAGGCAAGGCCCCAAAGCTGCTGATCTACAGCGCCAG |
| | (LC DNA with | CTACCGCTACACCGGCGTGCCAAGCCGCTTCAGCGGCAGCGGCAGCGGCACCG |
| | SP) | ACTTCACCCTGACCATCAGCAGCCTGCAGCCAGAGGACTTCGCCACCTACTAC |
| | | TGCCAGCAGTACTACATCTACCCATACACCTTCGGCCAGGGTACCAAGCTGGA |
| | | GATCAAGCGCGCTGATGCTGCACCAACTGTATCCATCTTCCCACCATCCAGTG |
| | | AGCAGTTAACATCTGGAGGTGCCTCAGTCGTGTGCTTCTTGAACAACTTCTAC |
| | | CCCAAAGACATCAATGTCAAGTGGAAGATTGATGGCAGTGAACGACAAAATGG |
| | | CGTCCTGAACAGTTGGACTGATCAGGACAGCAAAGACAGCACCTACAGCATGA |
| | | GCAGCACCCTCACGTTGACCAAGGACGAGTATGAACGACATAACAGCTATACC |
| | | TGTGAGGCCACTCACAAGACATCAACTTCACCCATTGTCAAGAGCTTCAACAG |
| | | GAATGAGTGTTGA |
| 19 | HER2.2C4VH- | EVQLVESGGG LVQPGGSLRL SCAASGFTFT DYTMDWVRQA |
| | mIgG2aCH1- | PGKGLEWVAD VNPNSGGSIY NQRFKGRFTL SVDRSKNTLY |
| | 2C4VH.mIgG2a. | LQMNSLRAED TAVYYCARNL GPSFYFDYWG QGTLVTVSSA |
| | LALAPG.Knob | KTTAPSVYPL APVCGDTTGS SVTLGCLVKG YFPEPVTLTW |
| | (HC mature) | NSGSLSSGVH TFPAVLQSDL YTLSSSVTVT SSTWPSQSIT |
| | | CNVAHPASST KVDKKIEPRG PTIKPEVQLV ESGGGLVQPG |
| | | GSLRLSCAAS GFTFTDYTMD WVRQAPGKGL EWVADVNPNS |
| | | GGSIYNQRFK GRFTLSVDRS KNTLYLQMNS LRAEDTAVYY |
| | | CARNLGPSFY FDYWGQGTLV TVSSAKTTAP SVYPLAPVCG |
| | | DTTGSSVTLG CLVKGYFPEP VTLTWNSGSL SSGVHTFPAV |
| | | LQSDLYTLSS SVTVTSSTWP SQSITCNVAH PASSTKVDKK |
| | | IEPRGPTIKP CPPCKCPAPN AAGGPSVFIF PPKIKDVLMI |
| | | SLSPIVTCVV VDVSEDDPDV QISWFVNNVE VHTAQTQTHR |
| | | EDYNSTLRVV SALPIQHQDW MSGKEFKCKV NNKDLGAPIE |
| | | RTISKPKGSV RAPQVYVLPP PEEEMTKKQV TLWCMVTDFM |
| | | PEDIYVEWTN NGKTELNYKN TEPVLDSDGS YFMYSKLRVE |
| | | KKNWVERNSY SCSVVHEGLH NHHTTKSFSR TPGK |
| 20 | HER2.2C4VH- | DIQMTQSPSS LSASVGDRVT ITCKASQDVS IGVAWYQQKP |
| | mIgG2aCH1- | GKAPKLLIYS ASYRYTGVPS RFSGSGSGTD FTLTISSLQP |
| | 2C4VH.mIgG2a. | EDFATYYCQQ YYIYPYTFGQ GTKLEIKRAD AAPTVSIFPP |
| | LALAPG.Knob | SSEQLTSGGA SVVCFLNNFY PKDINVKWKI DGSERQNGVL |
| | (LC mature) | NSWTDQDSKD STYSMSSTLT LTKDEYERHN SYTCEATHKT |
| | | STSPIVKSEN RNEC |
| 21 | HER2.7C2VH- | ATGGGATGGTCATGTATCATCCTTTTTCTAGTAGCAACTGCAACCGGTGTACA |
| | mIgG2aCH1- | TTCACAGGTCCAACTGCAGCAGCCTGGGGCTGAACTGGTGAGGCCTGGAGCTT |
| | 7C2VH.mIgG2a. | CAGTGAAGCTGTCCTGCAAGGCTTCTGGCTACTCCTTCACCGGCTACTGGATG |
| | LALAPG.Knob | AACTGGTTGAAGCAGAGGCCTGGACAAGGCCTTGAGTGGATTGGCATGATTCA |
| | (HC DNA with | TCCTTCCGATAGTGAAATTAGGGCAAATCAGAAATTCAGGGACAAGGCCACAT |
| | SP) | TGACTGTAGACAAGTCCTCCACCACAGCCTACATGCAACTCAGCAGCCCGACA |
| | | TCTGAGGACTCTGCGGTCTATTACTGTGCACGAGGGACTTACGACGGAGGCTT |
| | | TGAATACTGGGGCCAAGGCACCACTCTCACAGTCTCGAGTGCCAAGACCACCG |
| | | CCCCCAGCGTGTACCCCCTGGCCCCCGTGTGCGGCGACACCACCGGCAGCAGC |
| | | GTGACCCTGGGCTGCCTGGTGAAGGGCTACTTCCCCGAGCCCGTGACCCTGAC |
| | | CTGGAACAGCGGCAGCCTGAGCAGCGGCGTGCACACCTTCCCCGCCGTGCTGC |
| | | AGAGCGACCTGTACACCCTGAGCAGCAGCGTGACCGTGACCAGCAGCACCTGG |
| | | CCCAGCCAGAGCATCACCTGCAACGTGGCCCACCCCGCCAGCAGCACCAAGGT |
| | | GGACAAGAAGATCGAGCCCAGAGGCCCCACCATCAAGCCCCAGGTGCAGCTGC |
| | | AGCAGCCCGGCGCCGAGCTGGTGAGACCCGGCGCCAGCGTGAAGCTGAGCTGC |
| | | AAGGCCAGCGGCTACAGCTTCACCGGCTACTGGATGAACTGGCTGAAGCAGAG |
| | | ACCCGGCCAGGGCCTGGAGTGGATCGGCATGATCCACCCCAGCGACAGCGAGA |
| | | TCAGAGCCAACCAGAAGTTCAGAGACAAGGCCACCCTGACCGTGGACAAGAGC |
| | | AGCACCACCGCCTACATGCAGCTGAGCAGCCCCACCAGCGAGGACAGCGCCGT |
| | | GTACTACTGCGCCAGAGGCACCTACGACGGCGGCTTCGAGTACTGGGGCCAGG |
| | | GCACCACCCTGACCGTCTCGAGTGCCAAAACAACAGCCCCATCGGTCTATCCA |
| | | CTGGCTCCTGTGTGTGGAGATACAACTGGCTCCTCGGTGACTCTAGGATGCCT |
| | | GGTCAAGGGTTATTTCCCTGAGCCAGTGACCTTGACCTGGAACTCTGGATCCC |
| | | TGTCCAGTGGTGTGCACACCTTCCCAGCTGTCCTGCAGTCTGACCTCTACACC |
| | | CTCAGCAGCTCAGTGACTGTAACGTCGAGCACCTGGCCCAGCCAGTCCATCAC |
| | | CTGCAATGTGGCCCACCCGGCAAGCAGCACCAAGGTGGACAAGAAAATTGAGC |
| | | CCAGAGGACCCACAATCAAGCCCTGTCCTCCATGCAAATGCCCAGCACCTAAC |
| | | GCCGCGGGTGGACCATCCGTCTTCATCTTCCCTCCAAAGATCAAGGATGTACT |
| | | CATGATCTCCCTGAGCCCCATAGTCACATGTGTGGTGGTGGATGTGAGCGAGG |
| | | ATGACCCAGATGTCCAGATCAGCTGGTTTGTGAACAACGTGGAAGTACACACA |
| | | GCTCAGACACAAACCCATAGAGAGGATTACAACAGTACTCTACGCGTGGTCAG |
| | | TGCCCTCCCCATCCAGCACCAGGACTGGATGAGTGGCAAGGAGTTCAAATGCA |
| | | AGGTCAACAACAAAGACCTCGGAGCGCCCATCGAGAGAACCATCTCAAAACCC |
| | | AAAGGGTCAGTAAGAGCTCCACAGGTATATGTCTTGCCTCCACCAGAAGAAGA |
| | | GATGACTAAGAAACAGGTCACTCTGTGGTGCATGGTCACAGACTTCATGCCTG |
| | | AAGACATTTACGTGGAGTGGACCAACAACGGGAAAACAGAGCTAAACTACAAG |
| | | AACACTGAACCAGTCCTGGACTCTGATGGTTCTTACTTCATGTACAGCAAGCT |
| | | GAGAGTGGAAAAGAAGAACTGGGTGGAAAGAAATAGCTACTCCTGTTCAGTGG |
| | | TCCACGAGGGTCTGCACAATCACCACACGACTAAGAGCTTCTCCCGGACTCCG |
| | | GGTAAATGA |
| 22 | HER2.7C2VH- | ATGGGATGGTCATGTATCATCCTTTTTCTAGTAGCAACTGCAACCGGTGTACA |
| | mIgG2aCH1- | TTCAGACATCGTGCTGACCCAGAGCCCAGCCAGCCTGGTGGTGAGCCTGGGCC |
| | 7C2VH.mIgG2a. | AGCGCGCCACCATCAGCTGCCGCGCCAGCCAGAGCGTGAGCGGCAGCCGCTTC |
| | LALAPG.Knob | ACCTACATGCACTGGTATCAGCAGAAGCCAGGCCAGCCACCAAAGCTGCTGAT |
| | (LC DNA with | CAAGTACGCCAGCATCCTGGAGAGCGGCGTGCCAGCCCGCTTCAGCGGCGGCG |
| | SP) | GCAGCGGCACCGACTTCACCCTGAACATCCACCCAGTGGAGGAGGACGACACC |
| | | GCCACCTACTACTGCCAGCACAGCTGGGAGATCCCACCATGGACCTTCGGCGG |
| | | CGGTACCAAGCTGGAGATCAAGCGCGCTGATGCTGCACCAACTGTATCCATCT |
| | | TCCCACCATCCAGTGAGCAGTTAACATCTGGAGGTGCCTCAGTCGTGTGCTTC |
| | | TTGAACAACTTCTACCCCAAAGACATCAATGTCAAGTGGAAGATTGATGGCAG |
| | | TGAACGACAAAATGGCGTCCTGAACAGTTGGACTGATCAGGACAGCAAAGACA |
| | | GCACCTACAGCATGAGCAGCACCCTCACGTTGACCAAGGACGAGTATGAACGA |
| | | CATAACAGCTATACCTGTGAGGCCACTCACAAGACATCAACTTCACCCATTGT |
| | | CAAGAGCTTCAACAGGAATGAGTGTTGA |
| 23 | HER2.7C2VH- | QVQLQQPGAE LVRPGASVKL SCKASGYSFT GYWMNWLKQR |
| | mIgG2aCH1- | PGQGLEWIGM IHPSDSEIRA NQKFRDKATL TVDKSSTTAY |
| | 7C2VH.mIgG2a. | MQLSSPTSED SAVYYCARGT YDGGFEYWGQ GTTLTVSSAK |
| | LALAPG.Knob | TTAPSVYPLA PVCGDTTGSS VTLGCLVKGY FPEPVTLTWN |
| | (HC mature) | SGSLSSGVHT FPAVLQSDLY TLSSSVTVTS STWPSQSITC |
| | | NVAHPASSTK VDKKIEPRGP TIKPQVQLQQ PGAELVRPGA |
| | | SVKLSCKASG YSFTGYWMNW LKQRPGQGLE WIGMIHPSDS |
| | | EIRANQKFRD KATLTVDKSS TTAYMQLSSP TSEDSAVYYC |
| | | ARGTYDGGFE YWGQGTTLTV SSAKTTAPSV YPLAPVCGDT |
| | | TGSSVTLGCL VKGYFPEPVT LTWNSGSLSS GVHTFPAVLQ |
| | | SDLYTLSSSV TVTSSTWPSQ SITCNVAHPA SSTKVDKKIE |
| | | PRGPTIKPCP PCKCPAPNAA GGPSVFIFPP KIKDVLMISL |
| | | SPIVTCVVVD VSEDDPDVQI SWFVNNVEVH TAQTQTHRED |
| | | YNSTLRVVSA LPIQHQDWMS GKEFKCKVNN KDLGAPIERT |
| | | ISKPKGSVRA PQVYVLPPPE EEMTKKQVTL WCMVTDEMPE |
| | | DIYVEWTNNG KTELNYKNTE PVLDSDGSYF MYSKLRVEKK |
| | | NWVERNSYSC SVVHEGLHNH HTTKSFSRTP GK |
| 24 | HER2.7C2VH- | DIVLTQSPAS LVVSLGQRAT ISCRASQSVS GSRFTYMHWY |
| | mIgG2aCH1- | QQKPGQPPKL LIKYASILES GVPARFSGGG SGTDFTLNIH |
| | 7C2VH.mIgG2a. | PVEEDDTATY YCQHSWEIPP WTFGGGTKLE IKRADAAPTV |
| | LALAPG.Knob | SIFPPSSEQL TSGGASVVCF LNNFYPKDIN VKWKIDGSER |
| | (LC mature) | QNGVLNSWTD QDSKDSTYSM SSTLTLTKDE YERHNSYTCE |
| | | ATHKTSTSPI VKSENRNEC |
| 25 | IGF-1R.xIGF- | ATGGGATGGTCATGTATCATCCTTTTTCTAGTAGCAACTGCAACCGGTGTACA |
| | 1Rcixutumumab | TTCAGAGGTGCAGCTGGTGCAGAGCGGCGCCGAGGTGAAGAAGCCCGGCAGCA |
| | VH.mIgG2a.LAL | GCGTGAAGGTGAGCTGCAAGGCCAGCGGCGGCACCTTCAGCAGCTACGCCATC |
| | APG.Knob (HC | AGCTGGGTGAGACAGGCCCCCGGCCAGGGCCTGGAGTGGATGGGCGGCATCAT |
| | DNA with SP) | CCCCATCTTCGGCACCGCCAACTACGCCCAGAAGTTCCAGGGCAGAGTGACCA |
| | | TCACCGCCGACAAGAGCACCAGCACCGCCTACATGGAGCTGAGCAGCCTGAGA |
| | | AGCGAGGACACCGCCGTGTACTACTGCGCCAGAGCCCCCCTGAGATTCCTGGA |
| | | GTGGAGCACCCAGGACCACTACTACTACTACTACATGGACGTGTGGGGCAAGG |
| | | GCACCACCGTGACCGTCTCGAGTGCCAAAACAACAGCCCCATCGGTCTATCCA |
| | | CTGGCTCCTGTGTGTGGAGATACAACTGGCTCCTCGGTGACTCTAGGATGCCT |
| | | GGTCAAGGGTTATTTCCCTGAGCCAGTGACCTTGACCTGGAACTCTGGATCCC |
| | | TGTCCAGTGGTGTGCACACCTTCCCAGCTGTCCTGCAGTCTGACCTCTACACC |
| | | CTCAGCAGCTCAGTGACTGTAACGTCGAGCACCTGGCCCAGCCAGTCCATCAC |
| | | CTGCAATGTGGCCCACCCGGCAAGCAGCACCAAGGTGGACAAGAAAATTGAGC |
| | | CCAGAGGACCCACAATCAAGCCCTGTCCTCCATGCAAATGCCCAGCACCTAAC |
| | | GCCGCGGGTGGACCATCCGTCTTCATCTTCCCTCCAAAGATCAAGGATGTACT |
| | | CATGATCTCCCTGAGCCCCATAGTCACATGTGTGGTGGTGGATGTGAGCGAGG |
| | | ATGACCCAGATGTCCAGATCAGCTGGTTTGTGAACAACGTGGAAGTACACACA |
| | | GCTCAGACACAAACCCATAGAGAGGATTACAACAGTACTCTACGCGTGGTCAG |
| | | TGCCCTCCCCATCCAGCACCAGGACTGGATGAGTGGCAAGGAGTTCAAATGCA |
| | | AGGTCAACAACAAAGACCTCGGAGCGCCCATCGAGAGAACCATCTCAAAACCC |
| | | AAAGGGTCAGTAAGAGCTCCACAGGTATATGTCTTGCCTCCACCAGAAGAAGA |
| | | GATGACTAAGAAACAGGTCACTCTGTGGTGCATGGTCACAGACTTCATGCCTG |
| | | AAGACATTTACGTGGAGTGGACCAACAACGGGAAAACAGAGCTAAACTACAAG |
| | | AACACTGAACCAGTCCTGGACTCTGATGGTTCTTACTTCATGTACAGCAAGCT |
| | | GAGAGTGGAAAAGAAGAACTGGGTGGAAAGAAATAGCTACTCCTGTTCAGTGG |
| | | TCCACGAGGGTCTGCACAATCACCACACGACTAAGAGCTTCTCCCGGACTCCG |
| | | GGTAAATGA |
| 26 | IGF-1R.xIGF- | ATGGGATGGTCATGTATCATCCTTTTTCTAGTAGCAACTGCAACCGGTGTACA |
| | 1Rcixutumumab | TTCAAGCAGCGAGCTGACCCAGGACCCCGCCGTGAGCGTGGCCCTGGGCCAGA |
| | VH.mIgG2a.LAL | CCGTGAGAATCACCTGCCAGGGCGACAGCCTGAGAAGCTACTACGCCACCTGG |
| | APG.Knob (LC | TACCAGCAGAAGCCCGGCCAGGCCCCCATCCTGGTGATCTACGGCGAGAACAA |
| | DNA with SP) | GAGACCCAGCGGCATCCCCGACAGATTCAGCGGCAGCAGCAGCGGCAACACCG |
| | | CCAGCCTGACCATCACCGGCGCCCAGGCCGAGGACGAGGCCGACTACTACTGC |
| | | AAGAGCAGAGACGGCAGCGGCCAGCACCTGGTGTTCGGCGGCGGCACCAAGCT |
| | | GACCGTGCTTGGCCAGCCCAAGTCCACTCCCACTCTCACCGTGTTTCCACCCT |
| | | CGAGTGAGGAGCTCAAGGAAAACAAAGCCACACTGGTGTGTCTGATTTCCAAC |
| | | TTTTCCCCGAGTGGTGTGACAGTGGCCTGGAAGGCAAATGGTACACCTATCAC |
| | | CCAGGGTGTGGACACTTCAAATCCCACCAAAGAGGGCAACAAGTTCATGGCGA |
| | | GCAGCTTCCTACATTTGACATCGGACCAGTGGAGATCTCACAACAGTTTTACC |
| | | TGTCAAGTTACACATGAAGGGGACACTGTGGAGAAGAGTCTGTCACGTGCTGA |
| | | CTGTCTCTGA |
| 27 | IGF-1R.xIGF- | EVQLVQSGAE VKKPGSSVKV SCKASGGTFS SYAISWVRQA |
| | 1Rcixutumumab | PGQGLEWMGG IIPIFGTANY AQKFQGRVTI TADKSTSTAY |
| | VH.mIgG2a.LAL | MELSSLRSED TAVYYCARAP LRFLEWSTQD HYYYYYMDVW |
| | APG.Knob (HC | GKGTTVTVSS AKTTAPSVYP LAPVCGDTTG SSVTLGCLVK |
| | mature) | GYFPEPVTLT WNSGSLSSGV HTFPAVLQSD LYTLSSSVTV |
| | | TSSTWPSQSI TCNVAHPASS TKVDKKIEPR GPTIKPCPPC |
| | | KCPAPNAAGG PSVFIFPPKI KDVLMISLSP IVTCVVVDVS |
| | | EDDPDVQISW FVNNVEVHTA QTQTHREDYN STLRVVSALP |
| | | IQHQDWMSGK EFKCKVNNKD LGAPIERTIS KPKGSVRAPQ |
| | | VYVLPPPEEE MTKKQVTLWC MVTDEMPEDI YVEWTNNGKT |
| | | ELNYKNTEPV LDSDGSYFMY SKLRVEKKNW VERNSYSCSV |
| | | VHEGLHNHHT TKSFSRTPGK |
| 28 | IGF-1R.xIGF- | SSELTQDPAV SVALGQTVRI TCQGDSLRSY YATWYQQKPG |
| | 1Rcixutumumab | QAPILVIYGE NKRPSGIPDR FSGSSSGNTA SLTITGAQAE |
| | VH.mIgG2a.LAL | DEADYYCKSR DGSGQHLVFG GGTKLTVLGQ PKSTPTLTVF |
| | APG.Knob (LC | PPSSEELKEN KATLVCLISN FSPSGVTVAW KANGTPITQG |
| | mature) | VDTSNPTKEG NKFMASSFLH LTSDQWRSHN SFTCQVTHEG |
| | | DTVEKSLSRA DCL |
| 29 | NIST.mIgG2a.L | ATGGGATGGTCATGTATCATCCTTTTTCTAGTAGCAACTGCAACCGGTGTACA |
| | ALAPG.Knob | TTCACAGGTGACCCTGAGGGAGTCCGGCCCCGCCCTGGTGAAGCCCACCCAGA |
| | (HC DNA with | CCCTGACCCTGACCTGCACCTTCTCCGGCTTCTCCCTGTCCACCGCCGGCATG |
| | SP) | TCCGTGGGCTGGATCAGGCAGCCCCCCGGCAAGGCCCTGGAGTGGCTGGCCGA |
| | | CATCTGGTGGGACGACAAGAAGCACTACAACCCCTCCCTGAAGGACAGGCTGA |
| | | CCATCTCCAAGGACACCTCCAAGAACCAGGTGGTGCTGAAGGTGACCAACATG |
| | | GACCCCGCCGACACCGCCACCTACTACTGCGCCAGGGACATGATCTTCAACTT |
| | | CTACTTCGACGTGTGGGGCCAGGGCACCACCGTGACCGTCTCGAGTGCCAAAA |
| | | CAACAGCCCCATCGGTCTATCCACTGGCTCCTGTGTGTGGAGATACAACTGGC |
| | | TCCTCGGTGACTCTAGGATGCCTGGTCAAGGGTTATTTCCCTGAGCCAGTGAC |
| | | CTTGACCTGGAACTCTGGATCCCTGTCCAGTGGTGTGCACACCTTCCCAGCTG |
| | | TCCTGCAGTCTGACCTCTACACCCTCAGCAGCTCAGTGACTGTAACGTCGAGC |
| | | ACCTGGCCCAGCCAGTCCATCACCTGCAATGTGGCCCACCCGGCAAGCAGCAC |
| | | CAAGGTGGACAAGAAAATTGAGCCCAGAGGACCCACAATCAAGCCCTGTCCTC |
| | | CATGCAAATGCCCAGCACCTAACGCCGCGGGTGGACCATCCGTCTTCATCTTC |
| | | CCTCCAAAGATCAAGGATGTACTCATGATCTCCCTGAGCCCCATAGTCACATG |
| | | TGTGGTGGTGGATGTGAGCGAGGATGACCCAGATGTCCAGATCAGCTGGTTTG |
| | | TGAACAACGTGGAAGTACACACAGCTCAGACACAAACCCATAGAGAGGATTAC |
| | | AACAGTACTCTACGCGTGGTCAGTGCCCTCCCCATCCAGCACCAGGACTGGAT |
| | | GAGTGGCAAGGAGTTCAAATGCAAGGTCAACAACAAAGACCTCGGAGCGCCCA |
| | | TCGAGAGAACCATCTCAAAACCCAAAGGGTCAGTAAGAGCTCCACAGGTATAT |
| | | GTCTTGCCTCCACCAGAAGAAGAGATGACTAAGAAACAGGTCACTCTGTGGTG |
| | | CATGGTCACAGACTTCATGCCTGAAGACATTTACGTGGAGTGGACCAACAACG |
| | | GGAAAACAGAGCTAAACTACAAGAACACTGAACCAGTCCTGGACTCTGATGGT |
| | | TCTTACTTCATGTACAGCAAGCTGAGAGTGGAAAAGAAGAACTGGGTGGAAAG |
| | | AAATAGCTACTCCTGTTCAGTGGTCCACGAGGGTCTGCACAATCACCACACGA |
| | | CTAAGAGCTTCTCCCGGACTCCGGGTAAATGA |
| 30 | NIST.mIgG2a.L | ATGGGATGGTCATGTATCATCCTTTTTCTAGTAGCAACTGCAACCGGTGTACA |
| | ALAPG.Knob | TTCAGACATCCAGATGACCCAGTCCCCCTCCACCCTGTCCGCCTCCGTGGGCG |
| | (LC DNA with | ACAGGGTGACCATCACCTGCTCCGCCTCCTCCAGGGTGGGCTACATGCACTGG |
| | SP) | TATCAGCAGAAGCCCGGCAAGGCCCCCAAGCTGCTGATCTACGACACCTCCAA |
| | | GCTGGCCTCCGGCGTGCCCTCCAGGTTCTCCGGCTCCGGCTCCGGCACCGAGT |
| | | TCACCCTGACCATCTCCTCCCTGCAGCCCGACGACTTCGCCACCTACTACTGC |
| | | TTCCAGGGCTCCGGCTACCCCTTCACCTTCGGCGGCGGTACCAAGGTGGAGAT |
| | | CAAGCGCGCTGATGCTGCACCAACTGTATCCATCTTCCCACCATCCAGTGAGC |
| | | AGTTAACATCTGGAGGTGCCTCAGTCGTGTGCTTCTTGAACAACTTCTACCCC |
| | | AAAGACATCAATGTCAAGTGGAAGATTGATGGCAGTGAACGACAAAATGGCGT |
| | | CCTGAACAGTTGGACTGATCAGGACAGCAAAGACAGCACCTACAGCATGAGCA |
| | | GCACCCTCACGTTGACCAAGGACGAGTATGAACGACATAACAGCTATACCTGT |
| | | GAGGCCACTCACAAGACATCAACTTCACCCATTGTCAAGAGCTTCAACAGGAA |
| | | TGAGTGTTGA |
| 31 | NIST.mIgG2a.L | QVTLRESGPA LVKPTQTLTL TCTFSGFSLS TAGMSVGWIR |
| | ALAPG.Knob | QPPGKALEWL ADIWWDDKKH YNPSLKDRLT ISKDTSKNQV |
| | (HC mature) | VLKVTNMDPA DTATYYCARD MIFNFYFDVW GQGTTVTVSS |
| | | AKTTAPSVYP LAPVCGDTTG SSVTLGCLVK GYFPEPVTLT |
| | | WNSGSLSSGV HTFPAVLQSD LYTLSSSVTV TSSTWPSQSI |
| | | TCNVAHPASS TKVDKKIEPR GPTIKPCPPC KCPAPNAAGG |
| | | PSVFIFPPKI KDVLMISLSP IVTCVVVDVS EDDPDVQISW |
| | | FVNNVEVHTA QTQTHREDYN STLRVVSALP IQHQDWMSGK |
| | | EFKCKVNNKD LGAPIERTIS KPKGSVRAPQ VYVLPPPEEE |
| | | MTKKQVTLWC MVTDFMPEDI YVEWTNNGKT ELNYKNTEPV |
| | | LDSDGSYFMY SKLRVEKKNW VERNSYSCSV VHEGLHNHHT |
| | | TKSFSRTPGK |
| 32 | NIST.mIgG2a.L | DIQMTQSPST LSASVGDRVT ITCSASSRVG YMHWYQQKPG |
| | ALAPG.Knob | KAPKLLIYDT SKLASGVPSR FSGSGSGTEF TLTISSLQPD |
| | (LC mature) | DFATYYCFQG SGYPFTFGGG TKVEIKRADA APTVSIFPPS |
| | | SEQLTSGGAS VVCFLNNFYP KDINVKWKID GSERQNGVLN |
| | | SWTDQDSKDS TYSMSSTLTL TKDEYERHNS YTCEATHKTS |
| | | TSPIVKSENR NEC |
| 33 | RNF43-104 | CAGGTGCAGCTGAAGGAGTCAGGACCTGGCCTGGTGCAGCCCTCACAGACCCT |
| | (variable | GTCTCTCACCTGCAGTGTCTCTGGGTTTTCATTAACCAGCTTCCATGTGCACT |
| | heavy chain | GGGTTCGACAGCCTCCAGGAAAAGGTCTGGAGTGGATGGGATTAATGTGGAGT |
| | (VH) DNA) | AATGGAGACACATCATATAATTCAGATCTCAAATCCCGACTGAGCATCAGCAG |
| | | GGACACCTCCAAGAGTCAAGTCTTCTTAAAAATGCACAGTCTGCAGACTGAAG |
| | | ACACAGCCACTTACTACTGTGCCAGAACAGATGTATACCACGGATTCGGTGCT |
| | | ATGGATGCCTGGGGTCAGGGAACTTCAGTCACTGTCTCCTCA |
| 34 | RNF43-104 | GACATCCAGATGACACAGTCTCCGTCATTTCTGTCTGCATCTCTTGGAAACAG |
| | (variable | CATCACCATCACTTGCCATGCCAGTCAGTTCATCAAGGGTTGGTTAGCCTGGT |
| | light chain | ACCAACAAAAGTCAGGGAATGCTCCTGAACTGTTAATTTATAAGTCATCTAGC |
| | (VL) DNA) | CTGCATTCAGGGGTTCCATCAAGATTCAGTGGCAGCGGATCTGGAACAGATTA |
| | | TATTCTCACTATCAGCAACCTACAGCCTGAAGATATTGCCACTTATTACTGTC |
| | | AGCATTATCAAGGCTTTCCGCTCACGTTCGGTTCTGGGACCAAGCTGGAGATC |
| | | AAA |
| 35 | RNF43-104 (VH | QVQLKESGPGLVQPSQTLSLTCSVSGFSLTSFHVHWVRQPPGKGLEWMGLMWS |
| | protein) | NGDTSYNSDLKSRLSISRDTSKSQVFLKMHSLQTEDTATYYCARTDVYHGFGA |
| | | MDAWGQGTSVTVSS |
| 36 | RNF43-104 (VL | DIQMTQSPSFLSASLGNSITITCHASQFIKGWLAWYQQKSGNAPELLIYKSSS |
| | protein) | LHSGVPSRFSGSGSGTDYILTISNLQPEDIATYYCQHYQGFPLTFGSGTKLEI |
| | | K |
| 37 | RNF43-106 (VH | CAGATCCAGTTGGGACAGTCTGGACCTGAGCTGACGAAGCCTGGAGAGTCAGT |
| | DNA) | GAAGATCTCCTGCAAGGCTTCTGGGTATACCTTCTCAGACTATCCAATACACT |
| | | GGGTGAAACAGGCTCCAGGAAAGGGCTTGAAGTGGATGGGCTGGATCAACACC |
| | | TATACTGGGAAGCCAACATTTGCTGATGACTTCAAAGGACGGTTTGTCTTCTC |
| | | TTTGGAAGCCTCTGCCAGCACTGCAAATTTGCAGATCAGCAACCTCAAAAATG |
| | | AGGACACGGCTACATATTTCTGTGCTCTGGGCCCCATTTCTACAGTCTTCTAT |
| | | GCTATGGATGCCTGGGGTCAAGGAACTTCAGTCACTGTCTCCTCA |
| 38 | RNF43-106 (VL | GACATCCAGATGACCCAGTCTCCTTCATTCCTGTCTGCATCTGTGGGAGACAG |
| | DNA) | AGTCACTATCAACTGCAAAGCAAGTCAGAATATTAACAAGTATTTAAACTGGT |
| | | ATCAGCAAAAGCTTGGAGAAGCTCCCAAACGCCTGATATATAATACAAACAAT |
| | | TTGCAAACAGGCATCCCATCAAGGTTCAGTGGCAGTGGATCTGGTACAGATTA |
| | | CACACTCACCATCAGCAGCCTGCAGCCTGAAGATTTTGCCACATATTTCTGCT |
| | | TGCAGCATAATAGTTTTCCGTACACGTTTGGAGCTGGGACCAAGCTGGAACTG |
| | | AAA |
| 39 | RNF43-106 (VH | QIQLGQSGPELTKPGESVKISCKASGYTFSDYPIHWVKQAPGKGLKWMGWINT |
| | prot) | YTGKPTFADDFKGREVESLEASASTANLQISNLKNEDTATYFCALGPISTVFY |
| | | AMDAWGQGTSVTVSS |
| 40 | RNF43-106 (VL | DIQMTQSPSFLSASVGDRVTINCKASQNINKYLNWYQQKLGEAPKRLIYNTNN |
| | prot) | LQTGIPSRFSGSGSGTDYTLTISSLQPEDFATYFCLQHNSFPYTFGAGTKLEL |
| | | K |
| 41 | RNF43-107 (VH | GAGGTGCAGCCGGTGGAGTCTGGGGGCGGCTTGGTGCAGCCTGGAAGGTCCCT |
| | DNA) | AAAACTCTCCTGCCCAGCCTCAGGATTCACTTTCAGTAACTATGACATGGCCT |
| | | GGGTCCGCCAGGCTCCAACGAAGGGTCTGGAGTGGGTCGCTTCCATTAGTCCT |
| | | AGTGGTGGTAGAACTTACTATCGCGACTCCGTGAAGGGCCGATTCACTATTTC |
| | | CAGAGATAATTCAAAAAGTACCCTATACTTGCAAATGGACAGTCTGAGATCTG |
| | | AGGACACCGCCACTTTTTACTGTGCAACACATCGGTACTACAGTTACTTTGAT |
| | | TTCTGGGGCCAAGGAGTCATGGTCACAGTCTCCTCA |
| 42 | RNF43-107 (VL | GACATCCAGATGACACAGTCTCCAGCTTCCCTGTCTGCATCTCTGGGAGAAAC |
| | DNA) | TATCTCCATCGATTGTCGAACAAGTGAGGACATTTACAATAATTTAGCGTGGT |
| | | ATCAGCAGAAGTCAGGGAGATCTCCTCAGCTCCTGATCTCTGCTACAAATAGG |
| | | TTGCAAGATGGGGTCCCATCACGGTTCAGTGGCAGTGGTTCTGGCACACAGTA |
| | | TTCTCTCAAGATCAGCGGCATGCAACCTGAAGATGAAGGGGTTTATTTCTGTC |
| | | TACAGGGTTCCAAGTATCCGTATACGTTTGGAACTGGGACCAAGCTGGAACTG |
| | | AAA |
| 43 | RNF43-107 (VH | EVQPVESGGGLVQPGRSLKLSCPASGFTFSNYDMAWVRQAPTKGLEWVASISP |
| | prot) | SGGRTYYRDSVKGRFTISRDNSKSTLYLQMDSLRSEDTATFYCATHRYYSYFD |
| | | FWGQGVMVTVSS |
| 44 | RNF43-107 (VL | DIQMTQSPASLSASLGETISIDCRTSEDIYNNLAWYQQKSGRSPQLLISATNR |
| | prot) | LQDGVPSRFSGSGSGTQYSLKISGMQPEDEGVYFCLQGSKYPYTFGTGTKLEL |
| | | K |
| 45 | RNF43-108 (VH | CAGATACAACTGCAGCAATCTGGGGCTGAACTGGTGAAGCCTGGGTCCTCAGT |
| | DNA) | GACAGTTTCCTGCAAGGCTTCTGGCTACACCTTCACCAGTAACTTTATGCACT |
| | | GGATAAGACAGCAGCCTGGCAATGGCCTTGAGTGGATTGGGTGGATTTATCCT |
| | | GGCGATGGTGATGCAGAGACCAATCGAAAGTTCAATGGGAAGGCAACACTCAC |
| | | TGCAGACAAATCCTCCACCACAGCCTACTTGCAGCTCAGCAGCCTGACATCTG |
| | | AGGACTCTGCAGTCTATTTCTGTGCAAGAGGAGTGATGACGTCGTTCTGGTAC |
| | | TTTGACTTCTGGGGCCCAGGAACCATAGTCACCGTGTCCTCA |
| 46 | RNF43-108 (VL | GATGTCCAGATGACCCAGTCTCCATATAATCTTGCTGCCTCTCCTGGAGAAAG |
| | DNA) | TGTTTCCATCAATTGCAAGGCAAGTAAGAGCATTAGTAAGTATTTAGCCTGGT |
| | | ATCAACAGAAACCTGGGAAAACAAATAAGATTCTAATCTACGATGGGTCAACT |
| | | TTGCAATCTGGAATTCCATCGAGGTTCAGTGGCAGTGGATCTGGTACAGATTT |
| | | CACTCTCACCATCAGAGGCCTGGAGCCTGAAGATTTTGGACTCTATTACTGTC |
| | | AACAGCATAATGAACAGCCACCTACGTTCGGCTCGGGGACGAGGTTGGAAATA |
| | | AAA |
| 47 | RNF43-108 (VH | QIQLQQSGAELVKPGSSVTVSCKASGYTFTSNFMHWIRQQPGNGLEWIGWIYP |
| | prot) | GDGDAETNRKFNGKATLTADKSSTTAYLQLSSLTSEDSAVYFCARGVMTSFWY |
| | | FDFWGPGTIVTVSS |
| 48 | RNF43-108 (VL | DVQMTQSPYNLAASPGESVSINCKASKSISKYLAWYQQKPGKTNKILIYDGST |
| | prot) | LQSGIPSRFSGSGSGTDFTLTIRGLEPEDFGLYYCQQHNEQPPTFGSGTRLEI |
| | | K |
| 49 | RNF43-116 (VH | CAGGTCCAGCTGCAGCAGTCTGGAGCTGAGCTGGCAAAGCCTGGCTCTTCAGT |
| | DNA) | GAAGATTTCCTGCAAGGCTTCTGGCTACACCTTCACCAGCTACTATATAAGCT |
| | | GGATAAAGCAGACGACTGGACAGGACCTTGAGTATATTGGATATGTTAATACG |
| | | AGAAGTGGAGGTACTAACTACAATGAGAAGTTCAAGGTCAAGGCCACATTGAC |
| | | TGTAGACAATTCCTCCAGCACAGCCTTCATGCAACTCAGCAGCCTGACACCTG |
| | | ACGACTCTGCGGTCTATTACTGTGCAAGAGGCAGAACGGACTGGGGCCAAGGC |
| | | ACTCTGGTCACTGTCTCTTCA |
| 50 | RNF43-116 (VL | GACATCCAGATGACCCAGTCTCCTTCACTCCTGTCTGCATCTGTGGGAGACAG |
| | DNA) | AGTCACTGTAAACTGCAAAGCAAGTCAGAATATTTATAAGAACTTAGAGTGGT |
| | | ATCAGCAAAAGCATGGAGAAGCTCCAAAACTCCTGATATATTATACAAACAAT |
| | | TTGCAAACGGGCATCTCATCAAGGTTCAGTGGCAGTGGATCTGGTACAGATTA |
| | | CACACTCACCATCAGCAGCCTGCAGCCTGAAGATGTTGCCACATATTACTGCT |
| | | TTCAGTATAACAGCGGGTACACGTTTGGAGCTGGGACCAAGCTGGAACTGAAA |
| 51 | RNF43-116 (VH | QVQLQQSGAELAKPGSSVKISCKASGYTFTSYYISWIKQTTGQDLEYIGYVNT |
| | prot) | RSGGTNYNEKFKVKATLTVDNSSSTAFMQLSSLTPDDSAVYYCARGRTDWGQG |
| | | TLVTVSS |
| 52 | RNF43-116 (VL | DIQMTQSPSLLSASVGDRVTVNCKASQNIYKNLEWYQQKHGEAPKLLIYYTNN |
| | prot) | LQTGISSRFSGSGSGTDYTLTISSLQPEDVATYYCFQYNSGYTFGAGTKLELK |
| 53 | RNF43-117 (VH | CAGGTTTCTCTGAAAGAGTCTGGCCCTGGGATGTTGCAGCCCTCCCAGACCCT |
| | DNA) | CAGTCTGACTTGCACTTTCTCTGAGTTTTCACTGAGCACTTATGGTTTGGGTG |
| | | TGGGCTGGATTCGTCAGCCTTCAGGGAAGGGTCTGGAGTGGCTGGCGAACATT |
| | | TGGTGGGATGATGATAAGTACTACAATCCATCTCTGAAAAACCGCCTCACAAT |
| | | CTCCAAGGACACCTCCAACAACCAAGCATTCCTCAAGATCACCAATGTGGACA |
| | | CTGCTGATACTGCCACGTACTACTGTGCTCGGATAGACGGCAGGTGGCACTAC |
| | | TTTGATTACTGGGGCCAAGGAGACAGGGTCATAGTCTCCTCA |
| 54 | RNF43-117 (VL | GATATTGTCCTGACCCAGACTCCATCCATATTGTCTGCTACCATTGGACAATC |
| | DNA) | GGTCTCCATCTCTTGCAGGTCAACTCAGAGTCTCTTAGATAGTGATGGAAACA |
| | | CCTATTTATATTGGTTCCTACAGAGGCCAGGCCAGTCTCCACAGCGTCTAATT |
| | | TATTTGGTATCCAACCTGGGATCTGGGGTCCCCAACAGGTTCAGTGGCAGTGG |
| | | GTCAGGAACAGATTTCACACTCAAAATCAGTGGAGTGGAGGCTGAGGATTTGG |
| | | GATTTTATTACTGCATGCAAGGTGCCCATACTCCGCTCACGTTCGGTTCTGGG |
| | | ACCAAGCTGGAAATCAAA |
| 55 | RNF43-117 (VH | QVSLKESGPGMLQPSQTLSLTCTFSEFSLSTYGLGVGWIRQPSGKGLEWLANI |
| | prot) | WWDDDKYYNPSLKNRLTISKDTSNNQAFLKITNVDTADTATYYCARIDGRWHY |
| | | FDYWGQGDRVIVSS |
| 56 | RNF43-117 (VL | DIVLTQTPSILSATIGQSVSISCRSTQSLLDSDGNTYLYWFLQRPGQSPQRLI |
| | prot) | YLVSNLGSGVPNRFSGSGSGTDFTLKISGVEAEDLGFYYCMQGAHTPLTFGSG |
| | | TKLEIK |
| 57 | RNF43-123 VH | CAGATCCAGTTGGTACAGTCTGGACCTGAGCTGAAGAAGCCTGGAGAGTCAGT |
| | DNA) | GAAGATCTCCTGCAAGGCTTCTGGGTATACCTTCACAGACTATCCAATGCACT |
| | | GGGTGAAACAGGCTCCAGGAAAGGGCTTGAAGTGGATGGGCTGGATCAACACC |
| | | TATACTGGGAAGCCAACATATGCTGATGACTTCAAAGGACGGTTTGTCTTCTC |
| | | TTTGGAAGCCTCTGCCAACACTGCAAATTTACAGATCAGCAACCTCAAAAATG |
| | | ACGACACGGCTACATATTTCTGTGCTCTGGGCCCCATTTCTACAGTCTTCTAT |
| | | GCTGTGGATGCCTGGGGTCAAGGAACTTCAGTCACTGTCTCCTCA |
| 58 | RNF43-123 (VL | GACATCCAGATGACCCAGTCTCCTTCATTCCTGTCTGCATCTGTGGGAGACAG |
| | DNA) | AGTCACTATCAACTGCAAAGCAAGTCAGAATATTAACAAGTACTTAAACTGGT |
| | | ATCAGCAAAAGGTTGGAGAAGCTCCCAAGCGCCTGATGTATAATACAAACAAT |
| | | TTGCAAACAGGCATCCCATCAAGGTTCAGTGGCAGTGGATCTGGTACAGATTA |
| | | CACACTCACCATCAGCAGCCTGCAGCCTGAAGATTTTGCCACATATTTCTGCT |
| | | TGCAGCATAATAGTTTTCCGTACACGTTTGGACCTGGGACCGAGCTGGAACTG |
| | | AAA |
| 59 | RNF43-123 (VH | QIQLVQSGPELKKPGESVKISCKASGYTFTDYPMHWVKQAPGKGLKWMGWINT |
| | prot) | YTGKPTYADDFKGRFVFSLEASANTANLQISNLKNDDTATYFCALGPISTVFY |
| | | AVDAWGQGTSVTVSS |
| 60 | RNF43-123 (VL | DIQMTQSPSFLSASVGDRVTINCKASQNINKYLNWYQQKVGEAPKRLMYNTNN |
| | prot) | LQTGIPSRFSGSGSGTDYTLTISSLQPEDFATYFCLQHNSFPYTFGPGTELEL |
| | | K |
| 61 | RNF43-126 (VH | GAGGTGCAGCTTCAGGAGTCAGGACCTGGCCTTGTGAAACCCTCACAGTCACT |
| | DNA) | CTCCCTCACCTGTTCTGTCACTGGTTTCTCCATCACCAGTAGTCATAGATGGA |
| | | ACTGGATCCGGAAGTTCCCAGGAAATAAACTGGAGTGGATGGGATATATAAAC |
| | | AGTGCAGGCAGCACTAACTATAACCCGTCTCTCAAAAGTCGAATCTCCATTAC |
| | | TAGAGACACATCCAAGAATCAGTTCTTCCTGCAGGTGAACTCTGTAACTACTG |
| | | AGGACACAGCCACTTATTACTGTACGAGATTAGGACTACAGTATCGGGACTTT |
| | | GATTACTGGGGCCAAGGAGTCATGGTCACAGTCTCCTCA |
| 62 | RNF43-126 (VL | GACATCCAGATGACACAGTCTCCAGCTTCCCTGTCTGCATCTCTGGGAGAAAC |
| | DNA) | TATCTCCATCGAATGTCGAGCAAGTGACGACATTTACAGTAATTTAGCGTGGT |
| | | ATCAGCAGAAGTCAGGGAAATCTCCTCAGCTCCTGATCTATGCTGCAAATAGG |
| | | TTGAAAGATGGGGTCCCATCACGGTTCAGTGGCAGTGGATCTGGCACACAGTA |
| | | TTCTCTCAAGATCAGCGGCATGCAAACTGAAGATGAAGGGGATTATTTCTGTC |
| | | TACAGGCTTCCAAGTTTCCGCTCACGTTCGGTTCTGGGACCAAGCTGGAGATC |
| | | AAA |
| 63 | RNF43-126 (VH | EVQLQESGPGLVKPSQSLSLTCSVTGFSITSSHRWNWIRKFPGNKLEWMGYIN |
| | prot) | SAGSTNYNPSLKSRISITRDTSKNQFFLQVNSVTTEDTATYYCTRLGLQYRDF |
| | | DYWGQGVMVTVSS |
| 64 | RNF43-126 (VL | DIQMTQSPASLSASLGETISIECRASDDIYSNLAWYQQKSGKSPQLLIYAANR |
| | prot) | LKDGVPSRFSGSGSGTQYSLKISGMQTEDEGDYFCLQASKFPLTFGSGTKLEI |
| | | K |
| 65 | RNF43-128 (VH | GAGGTGCAGCTTCAGGAGTCAGGACCTGGCCTTGTGAAACCCTCTCAGTCACT |
| | DNA) | CTCCCTCACCTGTTCTGTCACTGGTTTCTCCATCACCAGTCATTATAAATGGA |
| | | ACTGGATCCGGAAGTTCCCAAGAAATAAACTGGAGTGGATGGGATATATAGAC |
| | | AATTCAGGCAGCACTAACTACATCCCGTCTCTCAAAAGTCGAATCTCCATTAC |
| | | TAGAGACACATCCAAGAATCAGTTCTTCCTGCAGGTGAACTCTGTAACTACTG |
| | | AGGACACAGCCACTTATTACTGTGCGAGGGACTGCCTGGACTGGGGCCAAGGA |
| | | GTCATGGTCACAGTCTCCTCA |
| 66 | RNF43-128 (VL | GATGTTGTGCTGACCCAGACTCCACCCACTTTATCGGCTACCATTGGACAATC |
| | DNA) | AGTCTCCATCTCTTGCAGGTCAAGTCAGAGTCTCTTACATAGTAATGGAAACA |
| | | CCTATTTACATTGGTTGATACAGAGGCCAGGCCAATCTCCACAGCTTCTAATT |
| | | TACTTGGTATCCAGACTGGAATCTGGGGTCCCCAACAGGTTCAGTGGCAGTGG |
| | | GTCAGGAACTGATTTCTCAGTCAAAATCAGTGGAGTAGAGGCTGAGGATTTGG |
| | | GAATTTATTACTGTGTGCAAGATACCCATACTCCGTGGACGTTCGGTGGAGGC |
| | | ACCAAGCTGGAATTGAAA |
| 67 | RNF43-128 (VH | EVQLQESGPGLVKPSQSLSLTCSVTGFSITSHYKWNWIRKFPRNKLEWMGYID |
| | prot | NSGSTNYIPSLKSRISITRDTSKNQFFLQVNSVTTEDTATYYCARDCLDWGQG |
| | | VMVTVSS |
| 68 | RNF43-128 (VL | DVVLTQTPPTLSATIGQSVSISCRSSQSLLHSNGNTYLHWLIQRPGQSPQLLI |
| | prot) | YLVSRLESGVPNRFSGSGSGTDFSVKISGVEAEDLGIYYCVQDTHTPWTFGGG |
| | | TKLELK |
| 69 | RNF43-129 (VH | GAGGTGCAGCTGGTGGAGTCTGGGGGAGGCTTGGTGCAGCCTGGAAGGTCCAT |
| | DNA) | GAAACTCTCCTGTGCAGCCTCAGGATTCACTTTCAGTAACTATGACATGGCCT |
| | | GGGTCCGCCAGGCTCCAAAGAAGGGTCTGGAGTGGGTCGCAAGTATTACTTAT |
| | | GATGTTAGTAGCACTTACTATCGAGACTCCGTGAAGGGCCGATTCACTATCTC |
| | | CAGAGATAATGCAAAAAGCACCCTATATTTGCAAATGGACAGTCTGAGGTCTG |
| | | AGGACACGGCCACTTATTACTGTACAACATTGGATCGGGCGGGTTATTACTAT |
| | | GATGGTTATGGCTTCTATTGGTTTGCCAACTGGGGCCAAGGCACTCTGGTCAC |
| | | TGTCTCTTCA |
| 70 | RNF43-129 (VL | GACATTCAGATGACCCAGTCTCCATCCTCCATGTCTGTGTCTCTGGGAGACAC |
| | DNA) | AGTCACTATTACTTGCCGGGCAAGTCAGGACGTTAAAATTTATGTAAACTGGT |
| | | TCCAGCAGAAACCAGGGAAATCTCCCAGACAAATGATTTACCGTGCAACGAAC |
| | | TTGGCAGATGGGGTCTCATCAAGGTTCAGCGGCAGTAGGTCTGGATCAGATTA |
| | | TTTATTCACCATCAGCAGCCTGGAGTCTGAAGATGTGGCAGACTATCACTGTC |
| | | TACAGTATGATGAGTATCCTCCAACGTTCGGTTCTGGGACCAAGGTGGAGATC |
| | | AAA |
| 71 | RNF43-129 (VH | EVQLVESGGGLVQPGRSMKLSCAASGFTFSNYDMAWVRQAPKKGLEWVASITY |
| | prot) | DVSSTYYRDSVKGRFTISRDNAKSTLYLQMDSLRSEDTATYYCTTLDRAGYYY |
| | | DGYGFYWFANWGQGTLVTVSS |
| 72 | RNF43-129 (VL | DIQMTQSPSSMSVSLGDTVTITCRASQDVKIYVNWFQQKPGKSPRQMIYRATN |
| | prot) | LADGVSSRFSGSRSGSDYLFTISSLESEDVADYHCLQYDEYPPTFGSGTKVEI |
| | | K |
| 73 | RNF43-130 (VH | CAGGTTCAGCTGGTCCAATCTGGGGCTGAGGTGGTGAAGCCTGGGTCCTCAGT |
| | DNA | GAAAATTTCCTGCAAGGCTTCTGGCTACACCTTCACCGGTAACTTTATTCACT |
| | | GGATAAAACAGCAGCCTGGCAATGGCCTTGAGTGGATTGGGTGGATTTATCCT |
| | | GGAGATGGTGATACAACATACAATCAGAAGTTCAGTGGGAAGGCAACACTCAC |
| | | TGCAGACAAGTCCTCCAGCACAGCCTATATGCAGCTCAGCAACCTGACATCTG |
| | | AGGACTCTGCAGTCTATTTCTGTGCAAAGTGGAGGGACCTGAGCAGTGATTGG |
| | | TTTGCTTACTGGGGCCAAGGCACTCTGGTCACTGTCTCTTCC |
| 74 | RNF43-130 (VL | GACATTGTGATGACCCAGTCTCCAGTCTCCCTGACTGTGTCAGAAGGAGAGAT |
| | DNA) | GGTCACCATAAACTGCAAGTCCAGTCAGAGTCTTTTATCCAGTGGAAACCAAA |
| | | AGAACTACTTGGCCTGGTACCAGCAGAAACCAGGGCAGTCTCCAAGACTACTG |
| | | ATCTACTTTACATCCACTAGACAATCAGGGGTCCCTGATCGCTTCATAGGCAG |
| | | TGGATCTGGGACAGACTTCACTCTGACCATCAGCGATGTGCAGGCTGAAGACC |
| | | TGGCAGATTATTACTGCCTGCAGCATTACAGCTATCCGAACACGTTTGGAGCT |
| | | GGGACCAAGTTGGAACTGAAA |
| 75 | RNF43-130 (VH | QVQLVQSGAEVVKPGSSVKISCKASGYTFTGNFIHWIKQQPGNGLEWIGWIYP |
| | prot) | GDGDTTYNQKFSGKATLTADKSSSTAYMQLSNLTSEDSAVYFCAKWRDLSSDW |
| | | FAYWGQGTLVTVSS |
| 76 | RNF43-130 (VL | DIVMTQSPVSLTVSEGEMVTINCKSSQSLLSSGNQKNYLAWYQQKPGQSPRLL |
| | prot) | IYFTSTRQSGVPDRFIGSGSGTDFTLTISDVQAEDLADYYCLQHYSYPNTFGA |
| | | GTKLELK |
| 77 | RNF43-136 (VH | CAGGTTCAGCTGCAGCAATCTGGGGCTGAATTAGTGAAGCCTGGGTCCTCAGT |
| | DNA) | GAAAATTTCCTGCAAGGCTTCTGGCTACACCTTCACCAGTAAGTTTATGCACT |
| | | GGATAAAGCAGCAGCCTGGAAGTGGCCTTGAGTGGATTGGGTGGATTTATCCT |
| | | GAACATGGTAATACTAAGTACAATCAAAAGTTCGATGGGAAGGCAACACTCAC |
| | | TGCAGACAAATCCTCCAGCACAGCCTATATGCAACTCAGCAGCCTGACATCTG |
| | | GGGACTCTGCAGTCTATTTCTGTGCAAGACAAAGCAGCCAGGGGGCCTATGCT |
| | | ATGGATGCCTGGGGTCAAGGAACTTCAGTCACTGTCTCCTCA |
| 78 | RNF43-136 (VL | GACATCCAAATGACACAGTCTCCAGTTTCCCTGTCTGCATCTCTTGGACAAAC |
| | DNA) | TGTCTCCATCGAATGTCTAACAAGTGAGGGCATTCCCATTGATTTAGCGTGGT |
| | | ATCAGCAGAAGTCAGGGAAATCTCCTCAACTCCTTATCTATGCTGCAAGTAGG |
| | | TCACAAGACGGGGTCCCATCACGGTTCAGTGGCAGTGGATCTGGCACACGGTA |
| | | TTCTCTCAAGATCAGCGGCATGCAACCTGAAGATGAAGCAGATTATTTCTGTC |
| | | AACAGACTTACAAGTATCCATTCACGTTCGGCTCAGGGACGAGGTTGGAAATA |
| | | AAA |
| 79 | RNF43-136 (VH | QVQLQQSGAELVKPGSSVKISCKASGYTFTSKFMHWIKQQPGSGLEWIGWIYP |
| | prot) | EHGNTKYNQKFDGKATLTADKSSSTAYMQLSSLTSGDSAVYFCARQSSQGAYA |
| | | MDAWGQGTSVTVSS |
| 80 | RNF43-136 (VL | DIQMTQSPVSLSASLGQTVSIECLTSEGIPIDLAWYQQKSGKSPQLLIYAASR |
| | prot) | SQDGVPSRFSGSGSGTRYSLKISGMQPEDEADYFCQQTYKYPFTFGSGTRLEI |
| | | K |
| 81 | RNF43-145 (VH | GAGGTGCAGCTGAAGGAATCAGGACCTGGTCTGGTGCAGCCCTCACAGACCCT |
| | DNA) | GTCCCTCACCTGCACTGTCTCTGGATTCTCATTAACGGACTACAGTGTATACT |
| | | GGGTTCGCCAGCCTCCAGGAAAAGGTCTGGAGTGGATGGGAGTAATGTGGAGT |
| | | GGTGGAACCACAGCATATAATTCAGCTCTCAAATCCCGACTGAGCATCAGCAG |
| | | GGACACCTCCAAGAGCCAAGTTTTCTTAAAAATGAACAGTCTGCAAACTGAAG |
| | | ACACAGCCATTTACTACTGTACCAGAGGGGGCAAATATGATGGTACTTATTAC |
| | | TACGGCTGGTACTTTGACTTCTGGGGCCCAGGAACCATGGTCACCGTGTCCTC |
| | | A |
| 82 | RNF43-145 (VL | GACGTTGTGCTGACTCAGTCTCCAGCCACCCTGTCTGTGACTCCAGGAGAAAG |
| | DNA) | AATCAGTCTCTCCTGCCGGGCCAGCGAGAGTGTTGACACTTATCTATACTGGT |
| | | ATCAGCAAAAGCCAAATGAGTCTCCAAGGCTTCTCATTAAGTATGCTTCCCAG |
| | | TCCATCTCTGGAATACCCTCCAGGTTCAGTGGCAGTGGATCTGGGACAGATTT |
| | | CACTCTCAGTATCAATGGAGTAGAGCTTGAAGATTTATCAATTTATTACTGTC |
| | | AACAAGGTAACAGCTTGCCCCTCACGTTCGGCTCAGGGACGAAGTTGGAAATA |
| | | AAA |
| 83 | RNF43-145 (VH | EVQLKESGPGLVQPSQTLSLTCTVSGFSLTDYSVYWVRQPPGKGLEWMGVMWS |
| | prot) | GGTTAYNSALKSRLSISRDTSKSQVFLKMNSLQTEDTAIYYCTRGGKYDGTYY |
| | | YGWYFDFWGPGTMVTVSS |
| 84 | RNF43-145 (VL | DVVLTQSPATLSVTPGERISLSCRASESVDTYLYWYQQKPNESPRLLIKYASQ |
| | prot) | SISGIPSRFSGSGSGTDFTLSINGVELEDLSIYYCQQGNSLPLTFGSGTKLEI |
| | | K |
| 85 | RNF43-152 (VH | CAGGTTACTCTGAAAGAGTCTGGCCCTGGGATATTGCAGCCCTCCCAGACCCT |
| | DNA) | CAGTCTGACTTGCACCTTCTCTGGGTCTTCACTGAGCACTTATGGTATGGGTG |
| | | TGGGCTGGATTCGTCAGCCTTCAGGGAAGGGTCTGGAGTGGCTGGCAAACATT |
| | | TGGTGGGATGATGATAAGTACTACAATCCATCTCTGAAAAACCGGCTCACAAT |
| | | CTCCAAGGACACCTCCAACAACCAATCATTCCTCAAGATCGCCAATGTGGACA |
| | | CTGCAGATACTGCCACATACTACTGTGCTCGGATAGACGGTACTTATAACTAC |
| | | TTTGATTATTGGGGCCAAGGAGTCATGGTCACAGTCTCCTCA |
| 86 | RNF43-152 (VL | GATGTTGTGTTGACCCAGACTCCATCCAGACTGTCTGCTACCATTGGACAATC |
| | DNA) | GGTCTCCATCTCTTGCAGGTCAAGTCAGAGTCTCTTAGATAGTTATGGAAACA |
| | | CCTATTTATCTTGGTTCCTACAGAGGCCAGGCCAATCTCCACAGCGTCTAATT |
| | | TTTTTGGTGTCCAACCTGGGATCTGGGGTCCCCAACAGGGTCAGTGGCAGTGG |
| | | GTCAGGAACTGATTTCACACTCAAAATCAGTGGAGTGGAGGCTGAGGATTTGG |
| | | GAGTTTATTACTGCATGCAAACTACCCATGATCCTCTCACGTTCGGTTCTGGG |
| | | ACCAAGCTGGAGATCAAA |
| 87 | RNF43-152 (VH | QVTLKESGPGILQPSQTLSLTCTFSGSSLSTYGMGVGWIRQPSGKGLEWLANI |
| | prot) | WWDDDKYYNPSLKNRLTISKDTSNNQSFLKIANVDTADTATYYCARIDGTYNY |
| | | FDYWGQGVMVTVSS |
| 88 | RNF43-152 (VL | DVVLTQTPSRLSATIGQSVSISCRSSQSLLDSYGNTYLSWFLQRPGQSPQRLI |
| | prot) | FLVSNLGSGVPNRVSGSGSGTDFTLKISGVEAEDLGVYYCMQTTHDPLTFGSG |
| | | TKLEIK |
| 89 | RNF43-156 (VH | GAGGTGCAGCTGAAGGAATCAGGACCTGGTCTGGTGCAGCCCTCACAGACCCT |
| | DNA) | GTCCCTCACCTGCACTGTCTCTGGATTCTCATTAACGGACTACAGTGTATACT |
| | | GGGTTCGCCAGCCTCCAGGAAAAGGTCTGGAGTGGATGGGAGTAATGTGGAGA |
| | | GGTGGAACCACAGCATATAATTCAGCTCTCAAATCCCGACTGAGCATCAGCAG |
| | | GGACACCTCCAAGAGCCAAGTTTTCTTAAAAATGAACAGTCTGCAAACTGAAG |
| | | ACACAGCCATTTACTACTGTACCAGAGGGGGCAAATATGATGGTACTTATTAC |
| | | TACGGCTGGTACTTTGACTTCTGGGGCCCAGGAACCATGGTCACCGTGTCCTC |
| | | A |
| 90 | RNF43-156 (VL | GACGTTGTGCTGACTCAGTCTCCAGCCACCCTGTCTGTGACTCCAGGAGAAAG |
| | DNA) | AATCAGTCTCTCCTGCCGGGCCAGCGAGAGTGTTGACACTTATCTATACTGGT |
| | | ATCAGCAAAAACCAAATGAGTCTCCAAGGCTTCTCATTAAGTATGCTTCCCAG |
| | | TCCATCTCTGGAATACCCTCCAGGTTCAGTGGCAGTGGATCTGGGACAGATTT |
| | | CACTCTCAGTATCAATGGAGTAGAGCTTGAAGATTTATCAATTTATTACTGTC |
| | | AACAAGGTAACAGCTTGCCCCTCACGTTCGGCTCAGGGACGAAGTTGGAAATA |
| | | AAA |
| 91 | RNF43-156 (VH | EVQLKESGPGLVQPSQTLSLTCTVSGFSLTDYSVYWVRQPPGKGLEWMGVMWR |
| | prot) | GGTTAYNSALKSRLSISRDTSKSQVFLKMNSLQTEDTAIYYCTRGGKYDGTYY |
| | | YGWYFDFWGPGTMVTVSS |
| 92 | RNF43-156 (VL | DVVLTQSPATLSVTPGERISLSCRASESVDTYLYWYQQKPNESPRLLIKYASQ |
| | prot) | SISGIPSRFSGSGSGTDFTLSINGVELEDLSIYYCQQGNSLPLTFGSGTKLEI |
| | | K |
| 93 | RNF43-168 (VH | CAGGTGCAGCTGAAGGAGTCAGGACCTGGTCTGGTGCAGCCCTCACAGACTTT |
| | DNA) | GTCTCTCACCTGCACTGTCTCTGGGTTCTCACTAAACAGGTATCATGTAAGCT |
| | | GGGTTCGCCAGCCTCCAGGAAAAGGTCTGGAGTGGATGGGAGTAGTATGGACT |
| | | GGTGGAAGCACAGCATATAATTCACTTCTCAAATCCCGACTGAGCATCAGCAG |
| | | GGACATCTCCAAGAGCCAAGTTTTCTTAAAAATGATCAGTCTGCAAACTGAAG |
| | | ACACAGCCACTTACTACTGTGTCAGTCAATACTACGGGTATACCTCCTACTTT |
| | | GATTACTGGGGCCAAGGAGTCATGGTCACAGTCTCCTCA |
| 94 | RNF43-168 (VL | GACATCCAGATGACACAGTCTCCTGCTTCCCTGTCTGCGTCTCCGGAAGAAAT |
| | DNA) | TGTCACGGTCACATGCCAGGCAAGCCAGGACATTGATAATTGGTTAACATGGT |
| | | ATCAGCAGAAACCAGGAAAATCTCCTCAACTCCTGATCTATAGTGCAACCAGC |
| | | TTGGCAGACGGGATCCCATCAAGGTTCAGCGGCAGTAGATCTGGTACACAGTA |
| | | TTCTCTTAAAATCGACAGACTACAGGTTGAAGATACTGGAATCTATTACTGTC |
| | | TACAGCGTTATGGTAATCCGTGGACGTTCGGTGGAGGCACCAAGCTGGAATTG |
| | | AAA |
| 95 | RNF43-168 (VH | QVQLKESGPGLVQPSQTLSLTCTVSGFSLNRYHVSWVRQPPGKGLEWMGVVWT |
| | prot) | GGSTAYNSLLKSRLSISRDISKSQVELKMISLQTEDTATYYCVSQYYGYTSYF |
| | | DYWGQGVMVTVSS |
| 96 | RNF43-168 (VL | DIQMTQSPASLSASPEEIVTVTCQASQDIDNWLTWYQQKPGKSPQLLIYSATS |
| | prot) | LADGIPSRFSGSRSGTQYSLKIDRLQVEDTGIYYCLQRYGNPWTFGGGTKLEL |
| | | K |
| 97 | RNF43-170 (VH | GAGGTACAACTGGTGGAGTCTGGGGGAGGCTTAGTGCCGCCTGGAAGGTCCCT |
| | DNA) | GAAACTCTCCTGTGTAGCCTCAGGATTCACTTTCAATGACTATTACATGGCCT |
| | | GGGTCCGCCAGGCTCCAACGAAGGGTCTGGAGTGGGTCGCATCCATTAGTTAT |
| | | GAGGGTCGTCGCACTTACTATGGAGACTCCGTGAAGGGCCGATTCACTATCTC |
| | | CAGAGATAATGCAAAAAGCACCCTATATCTGCAAATGAATAGTCTGAGGTCTG |
| | | ATGACACGGCCACTTATTATTGTGCAACACATAGCAACTATATCTTTGATTAC |
| | | TGGGGCCAGGGAGTCATGGTCACAGTCTCCTCA |
| 98 | RNF43-170 (VL | GATATCCGGATGACACAGTCTCCAGCTTCCCTGTCTGCATCTCTGGGAGAAAC |
| | DNA) | TGTCAACATCGAATGTCTAGCAAGTGAGGACATTTACAGTGATTTTGCATGGT |
| | | TTCAGCAGAAGCCAGGGCAATCTCCTCTGCTCCTGATCTATAATGGAAATAGT |
| | | TTGCAAAATGGGGTCCCTTCACGGTTTAGTGGCAGTGGATCTGGCACACAGTA |
| | | TTCTCTAAAAATCAACAGCCTCCAATCTGAAGATGTCGCGACTTATTTCTGTC |
| | | AACAATATAACAATTATCCTCCGACGTTCGGTGGAGGCACCAAGCTGGAATTG |
| | | AAA |
| 99 | RNF43-170 (VH | EVQLVESGGGLVPPGRSLKLSCVASGFTFNDYYMAWVRQAPTKGLEWVASISY |
| | prot) | EGRRTYYGDSVKGRFTISRDNAKSTLYLQMNSLRSDDTATYYCATHSNYIFDY |
| | | WGQGVMVTVSS |
| 100 | RNF43-170 (VL | DIRMTQSPASLSASLGETVNIECLASEDIYSDFAWFQQKPGQSPLLLIYNGNS |
| | prot) | LQNGVPSRFSGSGSGTQYSLKINSLQSEDVATYFCQQYNNYPPTFGGGTKLEL |
| | | K |
| 101 | RNF43-176 (VH | CAGGTGCAGCTGAAGGAGTCAGGACCTGGTCTGGTGCAGCCCTCACAGACTTT |
| | DNA) | GTCTCTCACCTGCACTGTCTCTGGGTTCTCACTAACCAGCTATCATGTAAGCT |
| | | GGGTTCGCCAGCCTCCAGGAAAAGGTCTGGAGTGGATGGGAGTAATATGGACT |
| | | GGTGGAAGCACAGCATATAATTCACTTCTCAAATCCCGACTGAGCATCAGCAG |
| | | GGACATCTCCAAGAGCCAAGTTTTCTTAAAAATGAACAGTCTGCAAACTGAAG |
| | | ACACAGCCACTTACTACTGTGCCAGTCAATACTACGGGTATACCTCCTATTTT |
| | | GATTACTGGGGCCAAGGAGTCATGGTCACAGTCTCCTCA |
| 102 | RNF43-176 (VL | GACATCCAGATGACACAGTCTCCTGTTTCCCTGTCTGCGTCTCCGGAAGAAAT |
| | DNA) | TGTCACGATCACATGCCAGGCAAGCCAGGACATTGATAATTGGTTAACATGGT |
| | | ATCAGCAGAAACCAGGGAAATCTCCTCAACTCCTGATCTATAGTGCAACCAGC |
| | | TTGGCAGACGGGATCCCATCAAGGTTCAGCGGCAGTAGATCTGGTACACAGTA |
| | | TTCTCTTAAGATCAGCAGACTACAGATTGAAGATACTGGGATCTATTACTGTC |
| | | TACAGCGTTATGGTAATCCGTGGACGTTCGGTGGAGGCACCAAGCTGGAATTG |
| | | AAA |
| 103 | RNF43-176 (VH | QVQLKESGPGLVQPSQTLSLTCTVSGFSLTSYHVSWVRQPPGKGLEWMGVIWT |
| | prot) | GGSTAYNSLLKSRLSISRDISKSQVFLKMNSLQTEDTATYYCASQYYGYTSYF |
| | | DYWGQGVMVTVSS |
| 104 | RNF43-176 (VL | DIQMTQSPVSLSASPEEIVTITCQASQDIDNWLTWYQQKPGKSPQLLIYSATS |
| | prot) | LADGIPSRFSGSRSGTQYSLKISRLQIEDTGIYYCLQRYGNPWTFGGGTKLEL |
| | | K |
| 105 | RNF43-177 (VH | CAGGTCAAGCTGCTGCAGTCTGGGGCTGCACTGGTGAAGCCTGGGGACTCTGC |
| | DNA) | GAAGATGTCTTGCAAAGCTTCTGGTTATACATTCACTGACTCCATTATACACT |
| | | GGGTGAAACAGAGTCATGGAAAGAGCCTTGAGTGGATTGGTTATATTAATCCT |
| | | TACAGTGGTGGTATTCAGTACAATGAAAAGTTCAAGAACAAGGCCACATTGAC |
| | | TGTAGACAAATCCAGCAGCACAGCCTATGTGGAGTTTAGCAGATTGACATCTG |
| | | AAGATTCTGCAATCTATTACTGTGCAAGAGAAATTCGGGGTATGGATGCCTGG |
| | | GGTCAAGGAACTTCAGTCACTGTCTCCTCA |
| 106 | RNF43-177 (VL | GACATCGTGCTGACTCAGTCTCCAGCCACCCTGTCTGTGACTCCAGGAGAGAG |
| | DNA) | TGTGAGTCTCTCCTGCAGGGCCAGTCAGGGTATTAGCACTAGTTTACACTGGT |
| | | ATCAGCAAAAATCAACTGAGTCTCCAAGGCTTCTCATCAAGTATGCTTCCCAG |
| | | TCCGTCTCTGGAATCCCCTCCAGGTTCAGTGGCAGTGGATCAGGGACAGATTT |
| | | CACTCTCAGTATCAACAGAGTAGAATCTGAAGATTTTTCAGTTTATTACTGTC |
| | | AACAGAGTTACAACTTGCCCCGCACGTTTGGACCTGGGACCAAGCTGGAACTG |
| | | AAA |
| 107 | RNF43-177 (VH | QVKLLQSGAALVKPGDSAKMSCKASGYTFTDSIIHWVKQSHGKSLEWIGYINP |
| | prot) | YSGGIQYNEKFKNKATLTVDKSSSTAYVEFSRLTSEDSAIYYCAREIRGMDAW |
| | | GQGTSVTVSS |
| 108 | RNF43-177 (VL | DIVLTQSPATLSVTPGESVSLSCRASQGISTSLHWYQQKSTESPRLLIKYASQ |
| | prot) | SVSGIPSRFSGSGSGTDFTLSINRVESEDFSVYYCQQSYNLPRTFGPGTKLEL |
| | | K |
| 109 | RNF43-179 (VH | GAGGTACAACTGGTGGAGTCTGGGGGAGGCTTAGTGCAGCCTGGAAGGTCCCT |
| | DNA) | GAAACTCTCCTGTGTAGCCTCAGGATTCACTTTCAATGACTATTACATGGCCT |
| | | GGGTCCGCCAGGCTCCAAAGAAGGGTCTGGAGTGGGTCGCATCCATTAATTAT |
| | | GAGGGTAGTCGCACTTACTATGGAGACTCCGTGAAGGGCCGATTCACTATCTC |
| | | CAGAGATAATGCAAAAAGCACCCTATATCTGCAAATGAATAGTCTGAGGTCTG |
| | | AGGACACGGCCACTTATTATTGTGCAACACATAGCAACTATATCTTTGATAAT |
| | | TGGGGCCAGGGAGTCATGGTCACAGTCTCCTCA |
| 110 | RNF43-179 (VL | GATATCCGGATGACACAGTCTCCAGCTTCCCTGTCTGCATCTCTGGGAGAAAC |
| | DNA) | TGTCAACATCGAATGTCTAGCAAGTGAGGACATTTACAGTGATTTTGCATGGT |
| | | TTCAGCAGAAGCCAGGGCAATCTCCTCTGCTCCTGATCTATAATGGAAATAAT |
| | | TTGCAAAATGGGGTCCCTTCACGGTTTAGTGGCAGTGGATCTGGCACACAGTA |
| | | TTCTCTAAAAATCAACAGCCTGCAATCTGAAGATGTCGCGACTTATTTCTGTC |
| | | AACAATATAACAATTATCCTCCGACGTTCGGTGGAGGCACCAAACTGGAATTG |
| | | AAA |
| 111 | RNF43-179 (VH | EVQLVESGGGLVQPGRSLKLSCVASGFTENDYYMAWVRQAPKKGLEWVASINY |
| | prot) | EGSRTYYGDSVKGRFTISRDNAKSTLYLQMNSLRSEDTATYYCATHSNYIFDN |
| | | WGQGVMVTVSS |
| 112 | RNF43-179 (VL | DIRMTQSPASLSASLGETVNIECLASEDIYSDFAWFQQKPGQSPLLLIYNGNN |
| | prot) | LQNGVPSRFSGSGSGTQYSLKINSLQSEDVATYFCQQYNNYPPTFGGGTKLEL |
| | | K |
| 113 | RNF43-180 (VH | GAGGCGCAATTGGTGGAGTCTGGGGGAGGCTTAGTGCGGCCTGGAAGGTCCCT |
| | DNA) | GAAACTCACCTGTGCAGCCTCAGGATTCACTTTCAGTCACTATGACATGGCCT |
| | | GGGTCCGCCAGGCTCCAGCGAGGGGTCTGGAGTGGGTCGCATCCATTGGTCCT |
| | | GGTGGTGGTCTCACTCACTATCGAGACTCCGTTAAGGGCCGATTCACTGTCTC |
| | | CAGAGATCATGCAGAAAGCAGCCTATACCTGCAAATGGACAGTCTGAGGTCTG |
| | | AGGACACGGCCACTTATTACTGTGCAAGACATTACGGGTGGTCCTACTTTGAT |
| | | TACTGGGGCCAAGGAGTCATGGTCACAGTCTCCTCA |
| 114 | RNF43-180 (VL | GACATCCAGATGACACAGTCTCCGTCATTTCTGTCTGCATCTCTTGGAGACAG |
| | DNA) | CATCACCATCACTTGCCATGCCAGTCAGAACATCAAGGGTTGGTTAGCCTGGT |
| | | ATCAACAAAAATCGGGGAATGCTCCTGAACTGTTGATTTATACGGCTTCTGGC |
| | | CTGCGATCAGGGGTTCCATCAAGATTCAGTGGCAGCGGATCTGGAACAGATTA |
| | | TATTCTCACTATCAGCAACCTACAGCCTGAAGATTTTGCCACTTATTATTGTC |
| | | AGCATTATCAAAGCTTTCCGAACACGTTTGGAGCCGGGACCAAGCTGGAACTG |
| | | AGA |
| 115 | RNF43-180 (VH | EAQLVESGGGLVRPGRSLKLTCAASGFTFSHYDMAWVRQAPARGLEWVASIGP |
| | prot) | GGGLTHYRDSVKGRFTVSRDHAESSLYLQMDSLRSEDTATYYCARHYGWSYED |
| | | YWGQGVMVTVSS |
| 116 | RNF43-180 (VL | DIQMTQSPSFLSASLGDSITITCHASQNIKGWLAWYQQKSGNAPELLIYTASG |
| | prot) | LRSGVPSRFSGSGSGTDYILTISNLQPEDFATYYCQHYQSFPNTFGAGTKLEL |
| | | R |
| 117 | RNF43-181 (VH | CAGATACAGCTGCAGCAATCTGGGGCTGAACTGGTGAAGCCTGGGTCCTCAGT |
| | DNA) | GAAAATTTCCTGCAAGTCTTCTGCCTACAGGTTCACCAGTAACTTTATTCACT |
| | | GGATAAGACAGCAGCCTGGCAATGGCCTTGAGTGGATTGGGTGGATTTATCCT |
| | | GGCGATGGTGATGCAGAGAACAATCAAAAGTTCAATGGGAAGGTAATACTCAC |
| | | TGCAGACAAATCGTCCAGCACAGCCTATATGCAGCTCAGCAGCCTGACATCTG |
| | | AGGACTCTGCGGTCTATTTCTGTGCAAGAGGAGTGATGACGTCGTTGTGGTAT |
| | | TTTGACTTTTGGGGCCCAGGAACCATGGTCACCGTGTCCTCA |
| 118 | RNF43-181 (VL | GATGTCCAGATGACCCAGTCTCCTTATAATCTTGCTGCCTCTCCTGGAGAAAG |
| | DNA) | TGTTTCCATCAATTGCAAGGCAAGTAAGAGCATTAGTAAGTATTTAGCCTGGT |
| | | ATCAACAGAAACCTGGGAAAACAAATAAGCTTCTTATCTACGATGGGTCCACT |
| | | TTGCAATCTGGAATTCCATCGAGGTTCAGTGGCAGTGGATCTGGTACAGATTT |
| | | CACTCTCACCATCAGAAGCCTGGAGCCTGAAGATTTTGGGCTCTATTACTGTC |
| | | AACAGCATCATGAACAGCCACCTACGTTCGGCTCGGGGACGAAGTTGGAAATA |
| | | AAA |
| 119 | RNF43-181 (VH | QIQLQQSGAELVKPGSSVKISCKSSAYRFTSNFIHWIRQQPGNGLEWIGWIYP |
| | prot) | GDGDAENNQKFNGKVILTADKSSSTAYMQLSSLTSEDSAVYFCARGVMTSLWY |
| | | FDFWGPGTMVTVSS |
| 120 | RNF43-181 (VL | DVQMTQSPYNLAASPGESVSINCKASKSISKYLAWYQQKPGKTNKLLIYDGST |
| | prot) | LQSGIPSRFSGSGSGTDFTLTIRSLEPEDFGLYYCQQHHEQPPTFGSGTKLEI |
| | | K |
| 121 | RNF43-186 (VH | CAGGTACAGCTACAGCAATCAGGGACTGAACTGGTGAAGCCTGCGTCCTCAGT |
| | DNA) | GAAAATTTCCTGCAAGGCTTCTGGCTACACCTTCACCACTAACTATATGCACT |
| | | GGATAAGGCAGCAGCCTGGAAATGGCCTTGAGTGGATTGGGTGGATTTATCCT |
| | | GGAAATGATAATACTAAGTACAATCAAAAGTTCGATGGGAAGGCAACACTCAC |
| | | TGCAGACAAATCCTCCAGCACAGCCTATATGCAGCTCAGCAGCCTGACATCTG |
| | | AGGACTCTGCAGTCTATTTCTGTGCAAGAGGGGGATTCGACTACGGAGGGTAT |
| | | TGGTTTGCTTACTGGGGCCAGGGCACTCCAGTCACTGTCTCTTCA |
| 122 | RNF43-186 (VL | GATATCCGGATGACACAGTCTCCAGCTTCCCTGTCTGCATCTCTGGGAGAAAC |
| | DNA) | TGTCAACATCGAATGTCTATCAAGTGAAGACATTTACAGTGAATTAGCATGGT |
| | | ATCAGCAGAAGCCAGGGAAATCTCCTCAGCTCCTGATGTATAATGGAAATAGC |
| | | TTGCAAAATGGGGTCCCTTCACGGTTTAGTGGCAGTGGATCTGGCACACAATA |
| | | TTCTCTAAAAATAAACAGCCTGCAATCTGAAGATGTCGCGACTTATTTCTGTC |
| | | AACAATATAGAAATTATCCATTCACGTTCGGCTCAGGGACGAAGTTGGAAATA |
| | | AAA |
| 123 | RNF43-186 (VH | QVQLQQSGTELVKPASSVKISCKASGYTFTTNYMHWIRQQPGNGLEWIGWIYP |
| | prot) | GNDNTKYNQKFDGKATLTADKSSSTAYMQLSSLTSEDSAVYFCARGGFDYGGY |
| | | WFAYWGQGTPVTVSS |
| 124 | RNF43-186 (VL | DIRMTQSPASLSASLGETVNIECLSSEDIYSELAWYQQKPGKSPQLLMYNGNS |
| | prot) | LQNGVPSRFSGSGSGTQYSLKINSLQSEDVATYFCQQYRNYPFTFGSGTKLEI |
| | | K |
| 125 | RNF43-187 (VH | CAGGTCCAGCTGCAGCAATCTGGGGCTGAACTGGTGAAGCCTGGGTCCTCAGT |
| | DNA) | GAAGATTTCCTGCAAGGCTTCTGGCTACACCTTCACCAGTAACTTTATGCACT |
| | | GGATAAAACAGCAGCCTGGAAATGGCTTTGAGTGGATTGGGTGGATTTATCCT |
| | | GGAGATGGTGATACAGACTACAATCAAAAGTTCAATGGGAGGGCAACACTCAC |
| | | TGCAGACAAATCCTCCAGCACAGCCTATATGCAGCTCAGCAGCCTGACATCTG |
| | | AGGACTCTGCAGTCTATTTCTGTGCAAGATGGGCGGGCCATCGTGAAGATTGG |
| | | TTTCCTTACTGGGGCCAAGGCACTCTGGTCACTGTCTCTTCA |
| 126 | RNF43-187 (VL | GACATTGTGATGACCCAATCTCCATCCTCCCTGGCTGTGTCAGCAGGAGAGAC |
| | DNA) | GGTCACTATAAACTGCAAGTCCAGTCAGAGTCTTTTATCCAGTGGAAACCAAA |
| | | AGAACTACTTGGCCTGGTACCAGCAGAAACCAGGGCAGTCTCCTAAACTGCTG |
| | | ATCTACTTGGCATCCACTAGGGAATCTGGAGTCCCTGATCGCTTCATAGGCAG |
| | | TGGATCTGGGACAGACTTCACTCTGACCATCAGCAGTGTGCAGGCTGAAGATC |
| | | TGGCAGACTATTACTGTCAGCAGCATTACACCTATCCGAACACGTTTGGACCT |
| | | GGGACCAAGTTGGAACTGAAA |
| 127 | RNF43-187 (VH | QVQLQQSGAELVKPGSSVKISCKASGYTFTSNFMHWIKQQPGNGFEWIGWIYP |
| | prot) | GDGDTDYNQKFNGRATLTADKSSSTAYMQLSSLTSEDSAVYFCARWAGHREDW |
| | | FPYWGQGTLVTVSS |
| 128 | RNF43-187 (VL | DIVMTQSPSSLAVSAGETVTINCKSSQSLLSSGNQKNYLAWYQQKPGQSPKLL |
| | prot) | IYLASTRESGVPDRFIGSGSGTDFTLTISSVQAEDLADYYCQQHYTYPNTFGP |
| | | GTKLELK |
| 129 | RNF43-196 (VH | CAGGTACAGCTGCAGCAATCTGGGGCTGAACTGGTGAAGCCTGGGTCCTCAGT |
| | DNA) | GAAAATTTCCTGCAAGGCTTCTGGCTTCACCTTCACCAGTAACTTTATGCACT |
| | | GGATAAAACAGCAGCCTGGAAATGGCCTTGAGTGGATTGGGTGGATTTATCCT |
| | | GGAGATGGTGATACAGACTACAATCAAAAGTTCAATGGGAAGGCAACTCTCAC |
| | | TGCAGACAAATCCTCCAGCACAGCCTATATGGAGCTCAGCAGCCTGACATCTG |
| | | AGGACTCTGCAGTCTATTTCTGTGCAAGAGCGTACTACGGTAGCTACTGGTAC |
| | | TTTGACTTCTGGGGCCCAGGAACCAAGGTCACCGTGTCCTCA |
| 130 | RNF43-196 (VL | GATGTCCAGATGACCCAGTCTCCACATAATCTTGCTGCCTCTCCTGGAGAAAG |
| | DNA) | TGTTTCCATCAATTGCAAGGCAAGTAAGAGCATTAGCAAGTATTTAGCCTGGT |
| | | ATCAGCAGAAACCTGGGAAAGTAAAAAAGCTTCTTATCTACGGTGGGTCAACT |
| | | TTGCAATCTGGAATTCCATCGAGGTTCAGTGGCAGTGGATCTGGTACAGATTT |
| | | CACTCTCACCATCAGCAGCCTGGAGCCTGAAGATTTTGGACTCTATTACTGTC |
| | | AACAGCATAATGAATACCCACCGACGTTCGGTGGAGGCACCAGGCTGGAATTG |
| | | CAA |
| 131 | RNF43-196 (VH | QVQLQQSGAELVKPGSSVKISCKASGFTFTSNFMHWIKQQPGNGLEWIGWIYP |
| | prot) | GDGDTDYNQKFNGKATLTADKSSSTAYMELSSLTSEDSAVYFCARAYYGSYWY |
| | | FDFWGPGTKVTVSS |
| 132 | RNF43-196 (VL | DVQMTQSPHNLAASPGESVSINCKASKSISKYLAWYQQKPGKVKKLLIYGGST |
| | prot) | LQSGIPSRFSGSGSGTDFTLTISSLEPEDFGLYYCQQHNEYPPTFGGGTRLEL |
| | | Q |
| 133 | RNF43-200 (VH | CAGGTGCAGCTGAAGGAGTCAGGACCTGGTCTGGTGCAGCCCTCACAGACTTT |
| | DNA) | GTCTCTCACCTGCACTGTCTCTGGGTTCTCACTAACCAGCTATCATATAAGCT |
| | | GGGTTCGCCAGCCTCCAGGAAAAGGTCTGGAGTGGATGGGAGTAATATGGACT |
| | | GGTGGAAGCACAGCATATAATTCACTTCTCAAATCCCGACTGAGCATCAGCAG |
| | | GGACATCTCCAAGAGCCAAGTCTTCTTAAAAATGAACAGTCTGCAAGCTGAAG |
| | | ACACAGCCACTTACTACTGTGCCGGATCATACTACGGGTATACCTCCTACTTT |
| | | GATTACTGGGGCCAAGGAGTCATGGTCACAGTCTCCTCA |
| 134 | RNF43-200 (VL | GACATCCAGATGACACAGTCTCCTGCTTCCCTGTCTGCGTCTCCGGAAGAAAT |
| | DNA) | TGTCACGATCACATGCCAGGCAAGCCAGGACATTGATAATTGGTTAGCATGGT |
| | | ATCAGCAGAAACCAGGGAAATCTCCTCAACTCCTGATCTATAGTGCAACCAGG |
| | | TTGGCAGACGGGATCCCATCAAGGTTCAGCGGCAGTAGATCTGGTATACAGTA |
| | | TTCTCTTAAGATCAACAGACTGCAGGTTGAAGATACTGGAATCTATTACTGTC |
| | | TACAGCGTTTTGGTAATCCTTGGACGTTCGGTGGAGGCACCAAGCTGGAATTG |
| | | AAA |
| 135 | RNF43-200 (VH | QVQLKESGPGLVQPSQTLSLTCTVSGESLTSYHISWVRQPPGKGLEWMGVIWT |
| | prot) | GGSTAYNSLLKSRLSISRDISKSQVFLKMNSLQAEDTATYYCAGSYYGYTSYF |
| | | DYWGQGVMVTVSS |
| 136 | RNF43-200 (VL | DIQMTQSPASLSASPEEIVTITCQASQDIDNWLAWYQQKPGKSPQLLIYSATR |
| | prot) | LADGIPSRFSGSRSGIQYSLKINRLQVEDTGIYYCLQRFGNPWTFGGGTKLEL |
| | | K |
| 137 | RNF43-201 (VH | CAGGTTACTCTGAAAGAGTATGGCCCTGGGATATTGCAGCCCTCCCAGACCCT |
| | DNA) | CAGTCTGACTTGCACTTTCTCTGGGTTTTCACTGAGGACTTATGGTATGGGTG |
| | | TGGGCTGGATTCGTCAGCCTTCAGGGAAGGGTCTGGAGTGGCTGACAAATATT |
| | | TGGTGGGATGATGATAAGTACTACAATCCATCTCTGAAAAACCGGCTCACAAT |
| | | CTCCAAGGACACCTCCAACAACCAAGCATTCCTCAAGATCACCAATGTGGACA |
| | | CTGAGGATAGTGCCACATACTATTGTGCTCGGATAGACGGTACTTTTAACTAC |
| | | TTTGATTATTGGGGCCAAGGAGTCATGGTCACAGTCTCCTCA |
| 138 | RNF43-201 (VL | GATGTTGTGCTGACCCAGACTCCTTCCAGATTGTCTGCTACCATTGGACAATC |
| | DNA) | GGTCTCCATCTCTTGCAGGTCAAGTCAGAGTCTCTTAGATAGTGATGGAAACA |
| | | CCTATTTATATTGGTTCCTACAGAGGCCAGGCCAATCTCCACAGCGTCTAATT |
| | | TATTTGGTATCCAACCTGGGATCTGGGGTCCCCAACAGGGTCAGTGGCAGTGG |
| | | GTCAGGAACAGATTTCACACTCAAAATCAGTGGAGTGGAGGCTGAGGATTTGG |
| | | GAATTTATTACTGCTTACAAGCTACCCATGCTCCTCTCACGTTCGGTTCTGGG |
| | | ACCAAGCTGGAGATCAAA |
| 139 | RNF43-201 (VH | QVTLKEYGPGILQPSQTLSLTCTFSGFSLRTYGMGVGWIRQPSGKGLEWLTNI |
| | prot) | WWDDDKYYNPSLKNRLTISKDTSNNQAFLKITNVDTEDSATYYCARIDGTFNY |
| | | FDYWGQGVMVTVSS |
| 140 | RNF43-201 (VL | DVVLTQTPSRLSATIGQSVSISCRSSQSLLDSDGNTYLYWFLQRPGQSPQRLI |
| | prot) | YLVSNLGSGVPNRVSGSGSGTDFTLKISGVEAEDLGIYYCLQATHAPLTFGSG |
| | | TKLEIK |
| 141 | RNF43-206 (VH | CAGGTGCAGCTGAAGGAGTCAGGACCTGGTCTGGTGCAGCCCTCACAGACTTT |
| | DNA) | GTCTCTCACCTGCACTGTCTCTGGGTTCTCACTAACCAGTTTTCATATAAGCT |
| | | GGGTTCGCCAGCCTCCAGGAAAAGGTCTGGAGTGGATGGGAATAATATGGACT |
| | | GGTGGAAGCACAGCATATAATTCACTTCTCAAATCCCGACTGAGCATCAGCAG |
| | | GGACATCTCCAAGAGCCAAGTCTTCTTAAAAATGAACAGTCTGCAAACTGAAG |
| | | ACACAGCCACTTACTACTGTGCCGGATCATATCACGGGTATACCTCCTACTTT |
| | | GATTACTGGGGCCAAGGAGTCATGGTCACAGTCTCCTCA |
| 142 | RNF43-206 (VL | GACATCCAGATGACTCAGTCTCCTGCTTCCCTGTCTGCGTCTCCGGAAGAAAT |
| | DNA) | TGTCACGATCACATGCCAGGCAAGCCAGGACATTGATAATTGGTTAGCATGGT |
| | | ATCAGCAGCAACCAGGGAAATCTCCTCAACTCCTGATCTATAGTGCAACTAGC |
| | | TTGGCAGACGGGATCCCATCAAGGTTCAGCGGCAGTAGATCTGGTACACAGTA |
| | | TTCTCTTAAGATCAACAGACTACAGGTTGAAGATACTGGAATCTATTACTGTC |
| | | TACAGCGTTATAGTAATCCTTGGACGTTCGGTGGAGGCACCAAGCTGGAATTG |
| | | AAA |
| 143 | RNF43-206 (VH | QVQLKESGPGLVQPSQTLSLTCTVSGFSLTSFHISWVRQPPGKGLEWMGIIWT |
| | prot) | GGSTAYNSLLKSRLSISRDISKSQVFLKMNSLQTEDTATYYCAGSYHGYTSYF |
| | | DYWGQGVMVTVSS |
| 144 | RNF43-206 (VL | DIQMTQSPASLSASPEEIVTITCQASQDIDNWLAWYQQQPGKSPQLLIYSATS |
| | prot) | LADGIPSRFSGSRSGTQYSLKINRLQVEDTGIYYCLQRYSNPWTFGGGTKLEL |
| | | K |
| 145 | RNF43-210 (VH | CAGATCCAGTTGGTACAGTCTGGACCTGAGCTGAAGAAGCCTGAAGAGTCAGT |
| | DNA) | GAAGATCTCCTGCAAGGCTTCTGGGTATATCTTCACAGACTATTTAATACACT |
| | | GGGTGAAGCAGGCTCCAGGAGAGGGCTTGAAGTGGATGGGCTGGATCAACACC |
| | | TATACTGGGAAGCCAACATATGCTGATGACTTCAAAGGACGGTTTGTCTTTTC |
| | | TTTGGAAGCCTCTGCCAGCACTGCAATTTTGCAGATCAGCAACCTCAAAAATG |
| | | AGGACACGGCTACATATTTCTGTGCTCTGGGCCCCATTACTACAGTCGTCTAT |
| | | GTCATGGATGCCTGGGGTCAAGGAACTTCAGTCACTGTCTCCTCA |
| 146 | RNF43-210 (VL | GACATCCAGATGACCCAGTCTCCTTCATTCCTGTCTGCATCTGTGGGAGACAG |
| | DNA | AGTCACTATCAACTGCAAAGCAAGTCAGAATATTAACAAGTACTTAAACTGGT |
| | | ATCAGCAAAAGCTTGGAGAAGCTCCCAAACGCCTGATATATAATACAAACAAT |
| | | TTGCAAACAGACATCCCATCAAGGTTCAGTGGCAGTGGATCTGGTGCAGATTA |
| | | CACACTCACCATCAACAGCCTGCAGCCTGAAGATTTTGCCACATATTTCTGCT |
| | | TGCAGCATAATAGTTTTCCGTACACGTTTGGAGCTGGGACCAAACTGGAACTG |
| | | AAA |
| 147 | RNF43-210 (VH | QIQLVQSGPELKKPEESVKISCKASGYIFTDYLIHWVKQAPGEGLKWMGWINT |
| | prot) | YTGKPTYADDFKGRFVESLEASASTAILQISNLKNEDTATYFCALGPITTVVY |
| | | VMDAWGQGTSVTVSS |
| 148 | RNF43-210 (VL | DIQMTQSPSFLSASVGDRVTINCKASQNINKYLNWYQQKLGEAPKRLIYNTNN |
| | prot) | LQTDIPSRFSGSGSGADYTLTINSLQPEDFATYFCLQHNSFPYTFGAGTKLEL |
| | | K |
| 149 | RNF43-213 (VH | CAGGTGCAGCTGAAGGAGTCAGGACCTGGTCTGGTGCAGCCCTCACAGACTTT |
| | DNA) | GTCTCTCACCTGCACTGTCTCTGGGTTCTCACTAACCAGGTATCATGTAAGCT |
| | | GGGTTCGCCAGCCTCCAGGAAAAGGTCTGGAGTGGATGGGAGTAATATGGACT |
| | | GGTGGAAGCACAGCATATAATTCACTTCTCAAATCCCGACTGAGCATCAGCAG |
| | | GGACATCTCCAAGAGCCAAGTTTTCTTAAAAATGAACAGTCTGCAAACTGAAG |
| | | ACACAGCCACTTACTACTGTGCCAGTCAATACTACGGGTATACCTCCTACTTT |
| | | GATTACTGGGGCCAAGGAGTCATGGTCACAGTCTCCGCA |
| 150 | RNF43-213 (VL | GACATCCAGATGACACAGTCTCCTGCTTCCCTGTCTGCGTCTCCGGAAGAAAT |
| | DNA) | TGTCACGATCACATGCCAGGCAAGCCAGGACATTGCTAATTGGTTAGCATGGT |
| | | ATCAGCAGAAACCAGGGAAATCTCCTCAACTCCTGATCTATAGTGCAACCAAC |
| | | TTGGCAGACGGGATCCCATCAAGGTTCAGCGGCAGTAGATCTGGTACACAGTA |
| | | TTCTCTTAAGATCAGCAGACTACAGGTTGAAGATACTGGAGTCTATTACTGTC |
| | | TACAGCGTTATGGTAATCCGTGGACGTTCGGTGGAGGCACCAACCTGGAATTG |
| | | AAA |
| 151 | RNF43-213 (VH | QVQLKESGPGLVQPSQTLSLTCTVSGFSLTRYHVSWVRQPPGKGLEWMGVIWT |
| | prot) | GGSTAYNSLLKSRLSISRDISKSQVFLKMNSLQTEDTATYYCASQYYGYTSYF |
| | | DYWGQGVMVTVSA |
| 152 | RNF43-213 (VL | DIQMTQSPASLSASPEEIVTITCQASQDIANWLAWYQQKPGKSPQLLIYSATN |
| | prot) | LADGIPSRFSGSRSGTQYSLKISRLQVEDTGVYYCLQRYGNPWTFGGGTNLEL |
| | | K |
| 153 | RNF43-217 (VH | GAGGTGCAGCTTCAGGAGTCAGGACCTGGCCTTGTGAAACCCTCACAGTCACT |
| | DNA) | CTCCCTCACCTGTTCTGTCACTGGTTTCTCCATCACCAGTAGTCATAGATGGA |
| | | ACTGGATCCGGAAGTTCCCAGGAAATAAACTGGAGTGGATGGGATATATAAAC |
| | | AGTGCAGGCAGCACTAACTATAACCCGTCTCTCAAAAGTCGAGTCTCCATTAC |
| | | TAGAGACGCATCCAAGAATCAGTTCTTCCTGCAGGTGAACTCTGTAACTACTG |
| | | AGGACACAGCCACTTATTACTGTACGAGATTAGGACTACAGTATCGGGACTTT |
| | | GATTACTGGGGCCAGGGAGTCATGGTCACAGTCTCCTCA |
| 154 | RNF43-217 (VL | GACATCCAGATGACACAGTCTCCAGCTTCCCTGTCTGCATCTCTGGGAGAAAC |
| | DNA) | TATCTCCATCGAATGTCGAGCAAGTGACGACATTTACAGTAATTTAGCGTGGT |
| | | ATCAGCAGAAGTCAGGGAAATCTCCTCAGCTCCTGATCTATGCTGCAAATAGG |
| | | TTGAAAGATGGGGTCCCATCACGGTTCAGTGGCAGTGGGTCTGGCACACAGTA |
| | | TTCTCTCAAGATCAGCGGCATGGAAACTGAAGATGAAGGGGATTATTTCTGTC |
| | | TACAGGCTTCCAAGTTTCCGCTCACGTTCGGTTCTGGGACCAAGCTGGAGATC |
| | | AAA |
| 155 | RNF43-217 (VH | EVQLQESGPGLVKPSQSLSLTCSVTGFSITSSHRWNWIRKFPGNKLEWMGYIN |
| | prot) | SAGSTNYNPSLKSRVSITRDASKNQFFLQVNSVTTEDTATYYCTRLGLQYRDE |
| | | DYWGQGVMVTVSS |
| 156 | RNF43-217 (VL | DIQMTQSPASLSASLGETISIECRASDDIYSNLAWYQQKSGKSPQLLIYAANR |
| | prot) | LKDGVPSRFSGSGSGTQYSLKISGMETEDEGDYFCLQASKFPLTFGSGTKLEI |
| | | K |
| 157 | RNF43-221 (VH | GAGGTGCAGCTGGTGGAGTCTGGCGGAGGCTTAGTGCAGCCTGGAAGGTCCCT |
| | DNA) | AAAACTCTCCTGTACAGCCTCAGGATTCACTCTCAGTGACTATGCCATGGCCT |
| | | GGGTCCGCCAGGCTCCAGAGAAAGGTCTGGAATGGGTCGCAACCATAAGTTAT |
| | | GATGGTAGTAGAACTTACTATGGAGGCTCCGTGAAGGGCCGATTCACTATCTC |
| | | CAGAGATAATGCAGAAAATATCCTATACCTGCAAATGGACAGTCTGAGGTCTG |
| | | CGGACACGGCCACTTATTATTGTGCAAACTCAGCAACTCCCCATTACTGGGGC |
| | | CAAGGAGTCATGGTCACAGTCTCCTCA |
| 158 | RNF43-221 (VL | GATGTTGTGATGACCCAGACTCCAGCGTCTTTGTCGCTTGCCATTGGACAACC |
| | DNA) | AGCCTCCATCTCTTGCAAGTCAAGCCAGAGCCTCTTAGATACTAGTCGAAAGA |
| | | CATTTTTGAATTGGATATTACAGAGGCCCGGCCAGTCTCCAAAGCGCCTAATC |
| | | TATCAGGTTTCCAAACTGTACTCAGAGGTTCCTGATAGGTTCAGTGGCAGTGG |
| | | ATCAGAGACAGAGTTTACTCTTAAAATCAGCAGAGTGGAGGCTGAGGATTTGG |
| | | GAGTGTATTACTGCTGGCAAGGTACACATTTTCCGGACACGTTTGGAGCTGGG |
| | | ACCAAGCTGGAACTGAAA |
| 159 | RNF43-221 (VH | EVQLVESGGGLVQPGRSLKLSCTASGFTLSDYAMAWVRQAPEKGLEWVATISY |
| | prot) | DGSRTYYGGSVKGRFTISRDNAENILYLQMDSLRSADTATYYCANSATPHYWG |
| | | QGVMVTVSS |
| 160 | RNF43-221 (VL | DVVMTQTPASLSLAIGQPASISCKSSQSLLDTSRKTFLNWILQRPGQSPKRLI |
| | prot) | YQVSKLYSEVPDRFSGSGSETEFTLKISRVEAEDLGVYYCWQGTHFPDTFGAG |
| | | TKLELK |
| 161 | RNF43-224 (VH | GAGGTGCAGCTGAAGGAATCAGGACCTGGTCTGGTGCAGCCCTCACAGACCCT |
| | DNA) | GTCCCTCACCTGCACTGTCTCTGGATTCTCATTGTCGGACTACAGTGTACACT |
| | | GGGTTCGCCAGCCTCCAGGAAAAGGTCTGGAGTGGATGGGAGTTATGTGGAGT |
| | | GGTGGAACCACAGCATATAATTCAGCTCTCAAATCCCGACTGAGCATCAGCAG |
| | | GGACACCTCCAAGAACCAAGTTCTCTTAGAAATGAACAGTCTGCAAACTGAAG |
| | | ACACAGCCATTTACTACTGTACCAGAGGGGGCAAATATGATGGTACTTATTAC |
| | | TACGGCTGGTACTTTGACTTCTGGGGCCCAGGAACCATGGTCACCGTGTCCTC |
| | | A |
| 162 | RNF43-224 (VL | GACGTTGTGCTGACTCAGTCTCCAGCCACCCTGTCTGTGACTCCAGGAGAAAG |
| | DNA) | AATCAGTCTCTCCTGTCGGGCCAGCGAGAGTGTTGACACTTATCTATCCTGGT |
| | | ATCAGCAAAAACCAAATGAGTCTCCAAGGCTTCTCATTAAGTATGCTTCCCAG |
| | | TCCATCTCTGGAATTCCCTCCAGGTTCAGTGGCAGTGGATCTGGGACAGATTT |
| | | CACTCTCAGTATCAATGGAGTAGAGCTTGAAGATTTATCAATTTATTACTGTC |
| | | AACAGGGTAACAGCTTGCCCCTCACGTTCGGCTCAGGGACGAAGTTGGAAATA |
| | | AAA |
| 163 | RNF43-224 (VH | EVQLKESGPGLVQPSQTLSLTCTVSGFSLSDYSVHWVRQPPGKGLEWMGVMWS |
| | prot) | GGTTAYNSALKSRLSISRDTSKNQVLLEMNSLQTEDTAIYYCTRGGKYDGTYY |
| | | YGWYFDFWGPGTMVTVSS |
| 164 | RNF43-224 (VL | DVVLTQSPATLSVTPGERISLSCRASESVDTYLSWYQQKPNESPRLLIKYASQ |
| | prot) | SISGIPSRFSGSGSGTDFTLSINGVELEDLSIYYCQQGNSLPLTFGSGTKLEI |
| | | K |
| 165 | RNF43-25 (VH | CAGGTCAAGCTGCTGCAGTCTGGGGCTGCACTGGTGAAGCCTGGGGACTCTGT |
| | DNA) | GAAGATGTCTTGCAAAGCTTCTGGTTATACATTCACTGACTACATTATACATT |
| | | GGGTGAAGCAGAGTCATGGAAGGAGCCTTGAGTGGATTGCTTATATTAATCCT |
| | | TACAGTGGTAGTACTAACTACAATGAAAAGTTCAAGAGCAAGGCCACATTGAC |
| | | TGTAGACAGATCCAACAACACAGCCTATATGGAGTTTAGCAGATTGACATCTG |
| | | AGGATTCTGCAATCTACTACTGTGCAAGAAAGAACTACGGAGGTTATGGAGTT |
| | | ATGGATGCCTGGGGTCAAGGAGCTTCAGTCACTGTCTCCTCA |
| 166 | RNF43-25 (VL | GACATCCAGATGACCCAGTCTCCTTCACTCCTGTCTGCATCTGTGGGAGACAG |
| | DNA) | AGTCACTCTCAGCTGCAAAGCAAGTCAGAATATTTATAAGAACTTAGCCTGGC |
| | | ATCAGCAAAAGCTTGGAGAAGCTCCCAAACTCCTGATTTATTATGCAAACACC |
| | | TTGCAAACGGGCATCCCATCAAGGTTCAGTGGCAGTGGATTTGGTACAGATTT |
| | | CACACTCACAATCAGCAGCCTGCAGCCTGAAGATGTTGCCACATATTTCTGCC |
| | | AGCAGTCTTCTAGCGGGTGGACGTTCGGTGGAGGCACCAAGCTGGAATTGAAA |
| 167 | RNF43-25 (VH | QVKLLQSGAALVKPGDSVKMSCKASGYTFTDYIIHWVKQSHGRSLEWIAYINP |
| | prot) | YSGSTNYNEKFKSKATLTVDRSNNTAYMEFSRLTSEDSAIYYCARKNYGGYGV |
| | | MDAWGQGASVTVSS |
| 168 | RNF43-25 (VL | DIQMTQSPSLLSASVGDRVTLSCKASQNIYKNLAWHQQKLGEAPKLLIYYANT |
| | prot) | LQTGIPSRFSGSGFGTDFTLTISSLQPEDVATYFCQQSSSGWTFGGGTKLELK |
| 169 | RNF43-31 (VH | GCGGTACAGCTGGTGGAGTCTGGGGGAGGCTCAGTGCAGCCTGGAAGGTCCAT |
| | DNA) | GCGGCTCTCCTGCGCAGCCTCAGGATTCACTTTCAGTAACTATGACATGACCT |
| | | GGGTCCGCCAGGCTCCAACGAAGGGTCTGGAGTGGGTCGCATCCATTACTTAT |
| | | GATGGTGGTAGTACTTACTCTCGAGACTCCGTGAAGGGCCGATTCACTATCTC |
| | | CAGAGATAATGCAAAAAGCACCCTATACCTGCAAATGGACAGTCTGAGGTCTG |
| | | AGGACACGGCCACTTATTACTGTACAACAGATCGGGGTAGATACCTACCCTAC |
| | | TACTTTGATTACTGGGGCCAAGGAGTCATGGTCACAGTCTCCGCA |
| 170 | RNF43-31 (VL | GATGTTGTGCTGACACAAACTCCAGTTTCCCTGTCTGTCACACTTGGAGATCA |
| | DNA) | AGCTTCTATATCTTGCAGGTCTAGTCAGAGCCTGGAATATAGTGATGGATATA |
| | | GTTATTTGGAATGGTACCTACAGAAGCCAGGCCAGTCTCCACAGCTCCTCATC |
| | | TATGAAGTTTCCAGCCGATTTTCTGGGGTCCCAGACAGGTTCATTGGCAGTGG |
| | | GTCAGGGACAGATTTCACCCTCAAGATCAGCAGAGTAGAGCCTGAGGACTTGG |
| | | GAGTTTATTACTGCTTCCAAGCTATACATGATCCCACGTTTGGAGCTGGGACC |
| | | AAGCTGGAACTGAAA |
| 171 | RNF43-31 (VH | AVQLVESGGGSVQPGRSMRLSCAASGFTFSNYDMTWVRQAPTKGLEWVASITY |
| | prot) | DGGSTYSRDSVKGRFTISRDNAKSTLYLQMDSLRSEDTATYYCTTDRGRYLPY |
| | | YFDYWGQGVMVTVSA |
| 172 | RNF43-31 (VL | DVVLTQTPVSLSVTLGDQASISCRSSQSLEYSDGYSYLEWYLQKPGQSPQLLI |
| | prot) | YEVSSRFSGVPDRFIGSGSGTDFTLKISRVEPEDLGVYYCFQAIHDPTFGAGT |
| | | KLELK |
| 173 | RNF43-33 (VH | CAGGAGCAGCTGAAGGAGTCAGGACCTGGTCTGGTGCAGCCCTCACAGACCCT |
| | DNA) | GTCCCTCACCTGCACTGTCTCTGGGTTCTCATTAACCAGCTATAATGTACACT |
| | | GGGTTCGACAGCCTCCAAGAAAGGGTCTGGAGTGGATGGGAAGAATGAGATAT |
| | | AATGGAGACACATCATATAATTCAGCTCTCAAATCCAGACTGAGCATTAGCAG |
| | | GGACACCTCCAAGAACCAAGTTTTCTTAAAAATGAACAGTCTGCAAACTGATG |
| | | ACACAGGCACTTACTACTGTGCCAGAGGGCCCCTCTGGGATTACTGGGGCCAA |
| | | GGAGTCATGGTCACAGTCTCCTCA |
| 174 | RNF43-33 (VL | GATGTTGTGCTGACCCAGACTCCACCCACTTTATCGGCTACCATTGGACAATC |
| | DNA) | AGTCTCCGTCTCTTGCAGGTCAAGTCAGAGTCTCTTACATAGTAATGGAAACA |
| | | CCTATTTAAATTGGTTGCTACAGAGGCCAGGCCAACCTCCACAACTTCTAATT |
| | | TATTTGGTATCCAGACTGGAATCTGGGGTCCCCAACAGGTTCAGTGGCAGTGG |
| | | GTCAGGAACTGATTTCACACTCAAAATCAGTGGAGTAGAGGCTGAGGATTTGG |
| | | GAGTTTATTACTGCGTGCAAAGTACCCATGCTCCGTACACGTTTGGAGCTGGG |
| | | ACCAAGCTGGAACTGAAA |
| 175 | RNF43-33 (VH | QEQLKESGPGLVQPSQTLSLTCTVSGFSLTSYNVHWVRQPPRKGLEWMGRMRY |
| | prot) | NGDTSYNSALKSRLSISRDTSKNQVFLKMNSLQTDDTGTYYCARGPLWDYWGQ |
| | | GVMVTVSS |
| 176 | RNF43-33 (VL | DVVLTQTPPTLSATIGQSVSVSCRSSQSLLHSNGNTYLNWLLQRPGQPPQLLI |
| | prot) | YLVSRLESGVPNRFSGSGSGTDFTLKISGVEAEDLGVYYCVQSTHAPYTFGAG |
| | | TKLELK |
| 177 | RNF43-35 (VH | CAGGTACAGTTGAAGGAGTCAGGACCTGGTCTGGTGCAGCCCTCGCAGACCCT |
| | DNA) | GTCCCTCACCTGCACTGTCTCTGGGTTCTCACTAACCACCTACAGTGTCCACT |
| | | GGGTTCGCCAACATTCAGGAAAGAATCTGGAATGGATGGGAAGAATGTGGACT |
| | | GCTGGAGACACATCATATAATTCAGCGTTCACATCCCGACTGAACATCTTCAG |
| | | GGACACCTCCAAGAGCCAAGTTTTCTTAAAAATGAACAGTCTGCAAACTGAGG |
| | | ACACAGGCACTTACTACTGTGCCAGATCCTCCTATACTTCGGGTTACCCTTTT |
| | | GATTCCTGGGGCCAAGGAGTCATGGTCACAGTCTCCTCA |
| 178 | RNF43-35 (VL | GACACTGTACTGACCCAGTCTCCTGCTTTGGCTGTGTCTCCAGGAGAGAGGGT |
| | DNA) | TACCATCTCCTGTAGGGCCAGTGAAAGTGTCAGTAAACTTATGCACTGGTACC |
| | | AACAGAGACCAGGACAACAACCCCAACTCCTCATCTATCTAACATCACACCTA |
| | | GCATCTGGGGTCCCTGCCAGGTTCAGTGGCAGTGGGTCTGGGACAGACTTCAC |
| | | CCTCACCATTGATCCTGTGGAGGCTGATGACACTGCAACCTATTACTGTCAGC |
| | | AGAGTCGGAATGATCCCACGTTTGGAGCTGGGACCAAGCTGGAACTGAAA |
| 179 | RNF43-35 (VH | QVQLKESGPGLVQPSQTLSLTCTVSGFSLTTYSVHWVRQHSGKNLEWMGRMWT |
| | prot) | AGDTSYNSAFTSRLNIFRDTSKSQVFLKMNSLQTEDTGTYYCARSSYTSGYPF |
| | | DSWGQGVMVTVSS |
| 180 | RNF43-35 (VL | DTVLTQSPALAVSPGERVTISCRASESVSKLMHWYQQRPGQQPQLLIYLTSHL |
| | prot) | ASGVPARFSGSGSGTDFTLTIDPVEADDTATYYCQQSRNDPTFGAGTKLELK |
| 181 | RNF43-38 (VH | GAAGTACAGCTTGTGGAGTCTGGAGGAGGCTTGGTGCAACCTGGGACTTCTCT |
| | DNA) | GAAAATCTCTTGTGTAGCCTCGGGATTCACTTTCAGTGACTACTGGATGAGCT |
| | | GGGTTCGCCAGACTCCTGGAAAGACCATGGAGTGGATTGGAGATATTAAATCT |
| | | GATGGCAGTTACACAAACTATGCACCATCCCTAAAGAATCGATTCACAATCTC |
| | | CAGAGACAATGCCAAGAGCACCCTGTACCTGCAGATGAACAATGTGAGATCTG |
| | | AAGACACAGCCACTTATTACTGTACTAGAGATGCATATAGGTACAACTACGTC |
| | | CACTATGGTTTGGATGCCTGGGGCCAAGGAGCTTCAGTCACTGTCTCCTCA |
| 182 | RNF43-38 (VL | CAGTTTGTGCTTACTCAGCCAAACTCTGTGTCTGCGAATCTCGGAAGCACAGT |
| | DNA) | CAAACTGTCTTGCAATCGCAGCACTGGTAACATTGGAAGCAATTATGTGAGCT |
| | | GGTACCAGCAGCATGAGGGAAGATCTCCCACCACTATGCTTTACAGGGATGGT |
| | | AAGAGACCAGATGGAATTCCTGACAGGTTCTCTGGCTCCATTGACGGATCTTC |
| | | CAACTCAGCCCTCCTGACAATCAACAATGTGCAGACTGAAGATGAAGCTGACT |
| | | ACTTCTGTCAGTCTTACAGTAGTGGTATTAAGTATATTTTCGGCGGTGGAACC |
| | | AAGCTCACTGTCCTA |
| 183 | RNF43-38 (VH | EVQLVESGGGLVQPGTSLKISCVASGFTFSDYWMSWVRQTPGKTMEWIGDIKS |
| | prot) | DGSYTNYAPSLKNRFTISRDNAKSTLYLQMNNVRSEDTATYYCTRDAYRYNYV |
| | | HYGLDAWGQGASVTVSS |
| 184 | RNF43-38 (VL | QFVLTQPNSVSANLGSTVKLSCNRSTGNIGSNYVSWYQQHEGRSPTTMLYRDG |
| | prot) | KRPDGIPDRFSGSIDGSSNSALLTINNVQTEDEADYFCQSYSSGIKYIFGGGT |
| | | KLTVL |
| 185 | RNF43-41 (VH | GAGGTGCACCTGGTGGAGTCTGGGGGAGGCTTAGTGCAGCCTGGAAGGTCCGT |
| | DNA) | GAAACTCTCCTGTACAGCCTCAGGATTCACTTTCAGTTACTATGACATGGCCT |
| | | GGGTCCGCCAGGCTCCAACGAAGGGTCTGGAGTGGGTCGCATCCCTTACTTAT |
| | | GATGGTAGTAGCACTTACTATCGAGACTCCGTGAAGGGCCGATTCACTATCTC |
| | | CAGAGATAATGCAAAAAGCACCCTATACCTGCAAATGGACAGTCTGAGGTCTG |
| | | AGGACACGGCCACTTATTACTGTACAACAGATCGGGGTAGATACCTACCCTAC |
| | | CACTTTGATTACTGGGGCCAAGGAGTCATGGTCACAGTCTCCTCA |
| 186 | RNF43-41 (VL | GATGTTGTGTTGACACAAACTCCAGTTTCCCTGTCTGTCACACTTGGAGATCA |
| | DNA) | AGCTTCTATATCTTGCAGGTCTAGTCAGAGCCTGGAATATAGTGATGGATACA |
| | | CTTATTTGGAATGGTACCTACAGAAGCCAGGCCAGTCTCCACAGCTCCTCATC |
| | | TATGAAGTTTCCACCCGATTTTCTGGGGTCCCAGACAGGTTCATTGGCAGTGG |
| | | GTCAGGGACAGATTTCACCCTTAAGATCAGCAGAGTAGAGCCTGAGGACTTGG |
| | | GAGTTTATTACTGCTTCCAAGCTACACATGATCCCACGTTTGGAGCTGGGACC |
| | | AAGCTCGAACTGAAA |
| 187 | RNF43-41 (VH | EVHLVESGGGLVQPGRSVKLSCTASGFTFSYYDMAWVRQAPTKGLEWVASLTY |
| | prot) | DGSSTYYRDSVKGRFTISRDNAKSTLYLQMDSLRSEDTATYYCTTDRGRYLPY |
| | | HFDYWGQGVMVTVSS |
| 188 | RNF43-41 (VL | DVVLTQTPVSLSVTLGDQASISCRSSQSLEYSDGYTYLEWYLQKPGQSPQLLI |
| | prot) | YEVSTRFSGVPDRFIGSGSGTDFTLKISRVEPEDLGVYYCFQATHDPTFGAGT |
| | | KLELK |
| 189 | RNF43-53 (VH | GAGGTGCAGTTGGTGGAGTCTGGGGGAGGCTTAGTGCAGCCTGGAAGGTCCCT |
| | DNA) | GAAACTCTCCTGTGGAGCCTCAGGATTCAGTTTCAGTACCTATTACATGGTCT |
| | | GGGTCCGCCAGGCTCCAACGAAGGGTCTGGAGTGGGTCGCATCCATTAGTCCT |
| | | AATGGTGGTAACACTTACTATCCAGACTCCGTGAAGGGCCGATTCACTATCTC |
| | | CAGAGATAATGCAAAAAGCACCCAATACCTGCAAATGGACAGTCTGAGGTCTG |
| | | AGGACACGGCCACTTATTACTGTGCAACAGATGGGTCGTATTACTATGATGGT |
| | | TATCGGTGGCTTGCTTACTGGGGCCAAGGCACTCTGGTCACTGTCTCTTCA |
| 190 | RNF43-53 (VL | CAGTTTGTGCTTACTCAGCCAAACTCTGTGTCTACGAATCTCGGAAGCACAGT |
| | DNA) | CAAACTGTCTTGCAAGCGCAGCACTGGTAACATTGGAAGCAATTATGTGAGCT |
| | | GGTACCAGCAGCATGAGGGAAGATCTCCCACCACTCTGATTTATAGGGATGAT |
| | | AAGAGACCAGATGGAGTTCCTGACAGGTTCTCTGGCTCCATTGACAGATCTTC |
| | | CAACTCAGCCCTCCTGACAATCAATAATGTGCAGACTGAAGATGAAGCTGGCT |
| | | ACTTCTGTCAGTCTTTCAGTAGTGGTCTGTTTATTTTCGGCGGTGGAACCAAG |
| | | CTCACTGTCCTA |
| 191 | RNF43-53 (VH | EVQLVESGGGLVQPGRSLKLSCGASGFSFSTYYMVWVRQAPTKGLEWVASISP |
| | prot) | NGGNTYYPDSVKGRFTISRDNAKSTQYLQMDSLRSEDTATYYCATDGSYYYDG |
| | | YRWLAYWGQGTLVTVSS |
| 192 | RNF43-53 (VL | QFVLTQPNSVSTNLGSTVKLSCKRSTGNIGSNYVSWYQQHEGRSPTTLIYRDD |
| | prot) | KRPDGVPDRFSGSIDRSSNSALLTINNVQTEDEAGYFCQSFSSGLFIFGGGTK |
| | | LTVL |
| 193 | RNF43-56 (VH | GAAGTACAGCTTGTGGAGTCTGGAGGAGGCTTGGTGCAACCTGGGACTTCTCT |
| | DNA) | GAAAATCTCTTGTGTAGCCTCGGGATTCACTTTCAGTGACTACTGGATGAGCT |
| | | GGGTTCGCCAGACTCCTGGAAGGACCATGGAGTGGATTGGAGATATTAAATAT |
| | | GATGGCAGTTACACAAACTATGCACCATCCCTAAAGAATCGATTCACAATCTC |
| | | CAGAGACAATGCCAAGAGTATCCTGTACCTGCAGATGAACAATGTGAGATCTG |
| | | AGGACACAGCCACTTATTACTGTACTAGAGATGCATATGGGTACAACTACGTC |
| | | CACTATGGTTTGGATGCCTGGGGCCAAGGAGCTTCAGTCACTGTCTCCTCA |
| 194 | RNF43-56 (VL | CAGTTTGTGCTTACTCAGCCAAACTCTGTGTCTTCGAATCTCGGAAGCACAGT |
| | DNA) | CAAACTGTCTTGCAATCGCAGCACTGGTAACATTGGAAGCAATTATGTGAGTT |
| | | GGTACCAGCAACATGAGGGAAGATCTCCCACCACTATGATTTATAGGGATGAT |
| | | AAGAGACCAGATGGAATTCCTGACAGGTTCTCTGGCTCCATTGACGGATCTTC |
| | | CAACTCGGCCCTCCTGACAATCAACAATGTGCAGACTGAAGATGAAGCTGACT |
| | | ACTTCTGTCAGTCTTACAGTAGTGATATTAAGTATATTTTCGGCGGTGGAACC |
| | | AAGCTCACTGTCCTA |
| 195 | RNF43-56 (VH | EVQLVESGGGLVQPGTSLKISCVASGFTFSDYWMSWVRQTPGRTMEWIGDIKY |
| | prot) | DGSYTNYAPSLKNRFTISRDNAKSILYLQMNNVRSEDTATYYCTRDAYGYNYV |
| | | HYGLDAWGQGASVTVSS |
| 196 | RNF43-56 (VL | QFVLTQPNSVSSNLGSTVKLSCNRSTGNIGSNYVSWYQQHEGRSPTTMIYRDD |
| | prot) | KRPDGIPDRESGSIDGSSNSALLTINNVQTEDEADYFCQSYSSDIKYIFGGGT |
| | | KLTVL |
| 197 | RNF43-61 (VH | GAGGTGCAGCTGGTGGAGTCTGGGGGAGACTTAGTGCAGCCTGGAAGGTCCCT |
| | DNA) | GACACTCTCCTGTGCAGCCTCAGGATTCATTTTCAGTGACTATTACATGGCCT |
| | | GGGTCCGCCAGGCTCCAAAGAAGGGTCTGGAGTGGGTCGCATCCATTACTTAT |
| | | GAGGGCAGTAGTACCTACTACGGAGACTCCGTGAAGGGCCGATTCACTATCTC |
| | | CAGAGATAATACAAAAAGCACCCTATATCTGCTAATGAACAGTCTGAGGTCTG |
| | | ACGACTCGGCCACTTACTATTGCGCAAGACATGGGGCACACTATACATATAAT |
| | | TACGCCTACTACTTTGATTACTGGGGCCAGGGAGTCATGGTCACAGTCTCCTC |
| | | A |
| 198 | RNF43-61 (VL | GACATCATGCTGACTCAGTCTCCAACTACCCTGTCTGTGATTCCAGGAGAGAA |
| | DNA) | CATCAGTCTCTCCTGCAGGGCCAGTCAGAGTGTTGGTACTAACTTACATTGGT |
| | | ATCAGCAAAAACCAAGTGAGTCTCCAAGGGTTCTCATCAACTATGCTTCCCAG |
| | | TCCATCTCTGGAATCCCCTCCAGGTTCAGTGGCAGTGGATCAGGGACAGATTT |
| | | CACTCTCAATATTAACAGAGTAGAGCCTGAAGATTCTTCAGTTTATTACTGTC |
| | | AACAGAGTAAGAGCTGGCCATTCACGTTCGGCTCAGGAACGAAGTTGGAAATA |
| | | AAA |
| 199 | RNF43-61 (VH | EVQLVESGGDLVQPGRSLTLSCAASGFIFSDYYMAWVRQAPKKGLEWVASITY |
| | prot) | EGSSTYYGDSVKGRFTISRDNTKSTLYLLMNSLRSDDSATYYCARHGAHYTYN |
| | | YAYYFDYWGQGVMVTVSS |
| 200 | RNF43-61 (VL | DIMLTQSPTTLSVIPGENISLSCRASQSVGTNLHWYQQKPSESPRVLINYASQ |
| | prot) | SISGIPSRFSGSGSGTDFTLNINRVEPEDSSVYYCQQSKSWPFTFGSGTKLEI |
| | | K |
| 201 | RNF43-67 (VH | GCGGTGCAGCTGGTGGAGTCTGGGGGAGGCTCAGTGCAGCCTGGAAGGTCCAT |
| | DNA) | GAGGCTCTCCTGCGCAGCCTCAGGATTCACTTTCAGTAACTATGACATGACCT |
| | | GGGTCCGCCAGGCTCCAACGAAGGGTCTGGAGTGGGTCGCATCCATTACTTCT |
| | | GATGGTGGTAGTACTTACTCTCGAGACTCCGTGAAGGGCCGATTCACTATCTC |
| | | CAGAGATAATGCAAAAAGCACCCTATACCTGCAAATGGACAGTCTGAGGTCTG |
| | | AGGACACGGCCACTTATTACTGTACAACAGATCGGGGTAGATACCTACCCTAC |
| | | TACTTTGATTACTGGGGCCAAGGAGTCATGGTCACAGTCTCCGCA |
| 202 | RNF43-67 (VL | GATGTTGTGCTGACACAAACTCCAGTTTCCCTGTCTGTCACAGTTGGAGATCA |
| | DNA) | AGCTTCTATATCTTGCAGGTCTAGTCAGAGCCTGGAATATAGTGATGGATATA |
| | | GTTATTTGGAATGGTACCTACAGAAGCCAGGCCAGTCTCCACAGCTCCTCATC |
| | | TATGAAGTTTCCAGCCGATTTTCTGGGGTCCCAGACAGGTTCATTGGCAGTGG |
| | | GTCAGGGACAGATTTCACCCTCAAGATCAGCAGAGTAGAGCCTGAGGACTTGG |
| | | GAGTTTATTACTGCTTCCAAGCTATACATGATCCCACGTTTGGAGCTGGGACC |
| | | AAGCTGGAACTGAAA |
| 203 | RNF43-67 (VH | AVQLVESGGGSVQPGRSMRLSCAASGFTFSNYDMTWVRQAPTKGLEWVASITS |
| | prot) | DGGSTYSRDSVKGRFTISRDNAKSTLYLQMDSLRSEDTATYYCTTDRGRYLPY |
| | | YFDYWGQGVMVTVSA |
| 204 | RNF43-67 (VL | DVVLTQTPVSLSVTVGDQASISCRSSQSLEYSDGYSYLEWYLQKPGQSPQLLI |
| | prot) | YEVSSRFSGVPDRFIGSGSGTDFTLKISRVEPEDLGVYYCFQAIHDPTFGAGT |
| | | KLELK |
| 205 | RNF43-69 (VH | GAGGTGCAGCTGGTGGAGTCTGGGGGAGGCTTAGTGCAGCCTGGAAGGTCCCT |
| | DNA) | GAAACTCTCCTGTGCAGCCTCAGGATTCACTTTCAGTGACTATTACATGGCCT |
| | | GGGTCCGCCAGGCTCCAAAGAAGGGTCTGGAGTGGGTCGCATCCATTACTTAT |
| | | GAGGGTAGTAGCACTTACTATGGAGACTCCGTGAAGGGCCGATTCACTATCTC |
| | | CAGAGATAATGCAAAAAGCACCCTATACCTGCAAATGAACAGTCTGAGGTCTG |
| | | AGGACACGGCCACTTTTTATTGTGCAAGACATCTGGACTATGGGTATGACTAC |
| | | GCCTGGTACTTTGACTTCTGGGGCCCAGGAACCATGGTCACCGTGTCCTCA |
| 206 | RNF43-69 (VL | GACATCGTGCTGACTCAGTCTCCAACCACCCTGTCTGTGACTCCAGGAGAGAC |
| | DNA) | AGTCAGTCTCTCCTGCAGGACTAGCCATAGTATTGGCACAAATCTACACTGGT |
| | | ATCAGCAAAAAACAAATGAGTCTCCAAGGCTTCTCATCAAGTATGCTTCCCAG |
| | | TCCATCTCTGGGATCCCCTCCAGGTTCAGTGCCAGTGGATCAGGGACAGATTT |
| | | TACTCTCAACATCAACAATGTGGAGTTTGATGATGTCTCAAGTTATTTTTGTC |
| | | AACAGACTCAAAGCTGGCCCCTCACGTTCGGTTCTGGGACCAAGCTGGAGATC |
| | | AAA |
| 207 | RNF43-69 (VH | EVQLVESGGGLVQPGRSLKLSCAASGFTFSDYYMAWVRQAPKKGLEWVASITY |
| | prot) | EGSSTYYGDSVKGRFTISRDNAKSTLYLQMNSLRSEDTATFYCARHLDYGYDY |
| | | AWYFDFWGPGTMVTVSS |
| 208 | RNF43-69 (VL | DIVLTQSPTTLSVTPGETVSLSCRTSHSIGTNLHWYQQKTNESPRLLIKYASQ |
| | prot) | SISGIPSRFSASGSGTDFTLNINNVEFDDVSSYFCQQTQSWPLTFGSGTKLEI |
| | | K |
| 209 | RNF43-71 (VH | CAGGTCAAGCTGCTGCAGTCTGGGGCTGCACTGGTGAAGCCTGGGGACTCTGT |
| | DNA) | GAAGATGTCTTGCAAAGCTTCTGGTTATACATTCACTGACCACATTATACACT |
| | | GGGTGAAGCAGAGTCATGGAAAGAGCCTTGAGTGGATTGGTTATATTAATCCT |
| | | TACAGTGGTAGTACTAACTACAATGAAAAGTTCAAGAGCAAGGCCACATTGAC |
| | | TGTAGACAGATCCAACAACACAGCCTATATGGAGTTTAGCAGATTGACATCTG |
| | | AGGATTCTGCAATCTATTACTGTGCAAGAAAGAACTACGGAGGTTATGGAGTT |
| | | ATGGATGCCTGGGGTCAAGGAGCTTCAGTCACTGTCTCCTCA |
| 210 | RNF43-71 (VL | GACATCCAGATGACCCAGTCTCCTTCACTCCTGTCTGCATCTGTGGGAGACAG |
| | DNA) | AGTCACTCTCAGCTGCAAAGCAAGTCAGAATATTTATAAGAACTTAGCCTGGC |
| | | ATCAGCAAAAGCTTGGAGAAGCTCCCAAACTCCTGATTTATTATGCAAACAGC |
| | | TTGCAAACGGGCATCCCATCAAGGTTCAGTGGCAGTGGATTTGGTACAGATTA |
| | | CACACTCACCATCAGCAGCCTGCAGCCTGAAGATGTTGCCACATATTTCTGCC |
| | | AGCAGTCTTCTAGCGGGTGGACGTTCGGTGGAGGCACCAAGCTGGAATTGAAA |
| 211 | RNF43-71 (VH | QVKLLQSGAALVKPGDSVKMSCKASGYTFTDHIIHWVKQSHGKSLEWIGYINP |
| | prot) | YSGSTNYNEKFKSKATLTVDRSNNTAYMEFSRLTSEDSAIYYCARKNYGGYGV |
| | | MDAWGQGASVTVSS |
| 212 | RNF43-71 (VL | DIQMTQSPSLLSASVGDRVTLSCKASQNIYKNLAWHQQKLGEAPKLLIYYANS |
| | prot) | LQTGIPSRFSGSGFGTDYTLTISSLQPEDVATYFCQQSSSGWTFGGGTKLELK |
| 213 | RNF43-74 (VH | GAGGTGCAGCTGGTGGAGTCTGGGGGAGACTTAGTGCAGCCTGGAAGGTCCCT |
| | DNA) | GACACTCTCCTGTGCAGCCTCAGGATTCACTTTCAGTGACTATTACATGGCCT |
| | | GGGTCCGCCAGGCTCCAAAGAAGGGTCTGGAGTGGGTCGCATCCATTACTTAT |
| | | GAGGGCAGTAGTACCTACTACGGAGACTCCGTGAAGGGCCGATTCACTATCTC |
| | | CAGAGATAATACAAAAAGCACCCTATATCTGCTAATGAACAGTCTGAGGTCTG |
| | | ACGACACGGCCACTTACTATTGCGCAAGACATGGGGCACACTATACATATAAT |
| | | TACGCCTACTACTTTGATTACTGGGGCCAGGGAGTCATGGTCACAGTCTCCTC |
| | | A |
| 214 | RNF43-74 (VL | GACATCATGCTGACTCAGTCTCCAACTACCCTGTCTGTGATTCCAGGAGAGAA |
| | DNA) | CATCAGTCTCTCCTGCAGGGCCAGTCAGAGTGTTGGTACTAACTTACATTGGT |
| | | ATCAGCAAAAACCAAGTGAGTCTCCAAGGGTTCTCATCAACTATGCTTCCCAG |
| | | TCCATCTCTGGAATCCCCTCCAGGTTCAGTGGCAGTGGATCAGGGACAGATTT |
| | | CACTCTCAATATTAACAGAGTAGAGCCTGAAGATTCTTCAGTTTATTACTGTC |
| | | AACAGAGTAAGAGCTGGCCATTCACGTTCGGCTCAGGCACGAAGTTGGAAATA |
| | | AAA |
| 215 | RNF43-74 (VH | EVQLVESGGDLVQPGRSLTLSCAASGFTFSDYYMAWVRQAPKKGLEWVASITY |
| | prot) | EGSSTYYGDSVKGRFTISRDNTKSTLYLLMNSLRSDDTATYYCARHGAHYTYN |
| | | YAYYFDYWGQGVMVTVSS |
| 216 | RNF43-74 (VL | DIMLTQSPTTLSVIPGENISLSCRASQSVGTNLHWYQQKPSESPRVLINYASQ |
| | prot) | SISGIPSRFSGSGSGTDFTLNINRVEPEDSSVYYCQQSKSWPFTFGSGTKLEI |
| | | K |
| 217 | RNF43-75 (VH | GAGGTGCAGTTGGTGGAGTCTGGGGGAGGCTTAGTGCAGCCTGGAAGGTCCCT |
| | DNA) | GAAACTCTCCTGTGCAGCCTCAGGATTCACTTTCAGTGACCATTACATGGCCT |
| | | GGGTCCGCCAGGCTCCAAAGAAGGGTCTGGAGTGGGTCGCATCCATTACTTAT |
| | | GAGGGTAGTAGCACTTACTATGGAGACTCCGTGAAGGGCCGATTCACTATCTC |
| | | CAGAGATAATGCAAAAAGCACCCTATACCTGCAAATGAACAGTCTGAGGTCTG |
| | | AGGACACGGCCACTTTTTATTGTGCAAGACATCTGGACTATGGGTATGACTAC |
| | | GCCTGGTCCTTTGACTTCTGGGGCCCAGGAACCATGGTCACCGTGTCCTCA |
| 218 | RNF43-75 (VL | GACATCGTGTTGACTCAGTCTCCAATCATCCTGTCTGTGACTCCAGGAGAGAC |
| | DNA) | AGTCAGTCTCTCCTGCAGGACTAGCCATAGTATTGGCACAAATCTACACTGGT |
| | | ATCAGCAAAAAACAAATGAGTCTCCAAGGCTTCTCATCAACTATGCTTCCCAG |
| | | TCCATCTCTGGGATCCCCTCCAGGTTCAGTGCCAGTGGATCAGGGACAGATTT |
| | | TACTCTCAACATCAACAATGTGGAGTTTGAAGATGTCGCAAGTTATTATTGTC |
| | | AACAGACTCAAAGGTGGCCCCTCACGTTCGGTTCTGGGACCAAGCTGGAGATC |
| | | AAA |
| 219 | RNF43-75 (VH | EVQLVESGGGLVQPGRSLKLSCAASGFTFSDHYMAWVRQAPKKGLEWVASITY |
| | prot) | EGSSTYYGDSVKGRFTISRDNAKSTLYLQMNSLRSEDTATFYCARHLDYGYDY |
| | | AWSFDFWGPGTMVTVSS |
| 220 | RNF43-75 (VL | DIVLTQSPIILSVTPGETVSLSCRTSHSIGTNLHWYQQKTNESPRLLINYASQ |
| | prot) | SISGIPSRFSASGSGTDFTLNINNVEFEDVASYYCQQTQRWPLTFGSGTKLEI |
| | | K |
| 221 | RNF43-76 (VH | GAAGTACAGCTTGTGGAGTCTGGAGGAGGCTTGGTGCAACCTGGGACTTCTCT |
| | DNA) | GAAAATCTCTTGTGTAGCCTCGGGATTCACTTTCAGTGACTACTGGATGAGCT |
| | | GGGTTCGCCAGACTCCTGGAAAGACCATGGAGTGGATTGGAGATATTAAATCT |
| | | GATGGCAGTTACACAAACTATGCACCATCCCTAAAGAATCGATTCACAATCTC |
| | | CAGAGACAATGCCAAGAGCACCCTGTACCTGCAGATGAACAATGTGAGATCTG |
| | | AAGACACAGCCACTTATTACTGTACTAGAGATGCATATAGGTACAACTACGTC |
| | | CACTATGGTTTGGATGCCTGGGGCCAAGGAGCTTCAGTCACTGTCTCCTCA |
| 222 | RNF43-76 (VL | CAGTTTGTGCTTACTCAGCCAAACTCTGTGTCTTCGAATCTCGGAAGCACAGT |
| | DNA) | CAAACTGTCTTGCAATCGCAGCACTGGTAACATTGGAAGCAATTATGTGAGCT |
| | | GGTACCAGCAGCATGAGGGAAGATCTCCCACCACTATGATTTACAGGGATGGT |
| | | AAGAGACCAGATGGAATTCCTGACAGGTTCTCTGGCTCCATTGACGGATCTTC |
| | | CAACTCAGCCCTCCTGACAATCAACAATGTGCAGACTGAAGATGAAGCTGACT |
| | | ACTTCTGTCAGTCTTACAGTAGTGGTATTAAGTATATTTTCGGCGGTGGAACC |
| | | AAGCTCACTGTCCTA |
| 223 | RNF43-76 (VH | EVQLVESGGGLVQPGTSLKISCVASGFTFSDYWMSWVRQTPGKTMEWIGDIKS |
| | prot) | DGSYTNYAPSLKNRFTISRDNAKSTLYLQMNNVRSEDTATYYCTRDAYRYNYV |
| | | HYGLDAWGQGASVTVSS |
| 224 | RNF43-76 (VL | QFVLTQPNSVSSNLGSTVKLSCNRSTGNIGSNYVSWYQQHEGRSPTTMIYRDG |
| | prot) | KRPDGIPDRFSGSIDGSSNSALLTINNVQTEDEADYFCQSYSSGIKYIFGGGT |
| | | KLTVL |
| 225 | RNF43-80 (VH | GAGGTGCAGCTGGTGGAGTCTGGAGGAGGCTTAGTCCAGCCTGGAAGGTCCCT |
| | DNA) | GAAAGTCTCCTGTTTAGCCTCTGGATTCAACTTCGGTGACTTTGGAATGAATT |
| | | GGATTCGCCAGGCTCCAGGGAAGGGGCTGGAGTGGGTTTCATATATTAGTAGT |
| | | ACCAGTGGTACAATCTACTATGCGGACACTGTGAAGGGTCGCTTCACCATTTC |
| | | CAGAGATAATGCCAAGAACACCCTTTATCTCCAACTGAGCAGTCTGAGGTCTG |
| | | AGGACACCGCCTTGTATTACTGTGCAAGACCGGCGGGAAGGTACTTTGATTAC |
| | | TGGGGCCAAGGAGTCTTGGTCACAGTCTCCTCA |
| 226 | RNF43-80 (VL | GACATTGTGATGACCCAATCTCCATCCTCTCTGGCTGTGTCAGCAGGAGAGAC |
| | DNA) | GGTCACTATAGACTGCAAGTCCAATCAGAGTCTTTTATACAGTGGAAACCAAA |
| | | AGAACTACTTGGCCTGGTACCAGCAGAAACCAGGGCAGTCTCCTAAACTGCTG |
| | | ATCTACTGGACTTCTACTAGGCAATCTGGTGTCCCTGATCGCTTCATAGGCAG |
| | | TGGATCTGGGACAGAGTTCACTCTGACCATCAGCAGTGTGCAGGCAGAAGATC |
| | | TGGCAATATATTACTGTCAGCAGTATTATCACACTCCATTCACGTTCGGCTCA |
| | | GGGACGAAATTGGAAATAAAA |
| 227 | RNF43-80 (VH | EVQLVESGGGLVQPGRSLKVSCLASGENFGDFGMNWIRQAPGKGLEWVSYISS |
| | prot) | TSGTIYYADTVKGRFTISRDNAKNTLYLQLSSLRSEDTALYYCARPAGRYFDY |
| | | WGQGVLVTVSS |
| 228 | RNF43-80 (VL | DIVMTQSPSSLAVSAGETVTIDCKSNQSLLYSGNQKNYLAWYQQKPGQSPKLL |
| | prot) | IYWTSTRQSGVPDRFIGSGSGTEFTLTISSVQAEDLAIYYCQQYYHTPFTFGS |
| | | GTKLEIK |
| 229 | RNF43-86 (VH | GAGGTGCAGCTGGTGGAGTCTGGGGGAGGCTTAGTGCAGCCTGGAAGGTCCCT |
| | DNA) | GAAACTCTCCTGTGCAGCCTCAGGATTCACTTTCACTAACTATTACATGGCCT |
| | | GGGTCCGCCAGGCTCCAACGAAGGGTCTGGAGTGGGTCGCGTCCATCAGTCCT |
| | | AGTGGTGATACCACTTACTATCCAGACTCCGTGAAGGGCCGATTCACTGTCTC |
| | | CAGAGATAATGCAAAAAGTACCCAATACCTGCAAATGGACAATCTGAGGTCTG |
| | | AGGACACGGCCACTTATTACTGTGCAAGAGACGCATATACATACTATGGATAT |
| | | AAGGGCTGGTACTTTGACTTCTGGGGCCCAGGAACCATGGTCACCGTGTCCTC |
| | | A |
| 230 | RNF43-86 (VL | CAGTTTGTGCTTACTCAGCCAAACTCTGTGTCTACGAATCTCGGAAGTGCAGT |
| | DNA) | CAAACTGTCTTGCAAGCGCAGCACTGATGACATTGGAAGCAATTATGTGAACT |
| | | GGTACCAGCAGCATGAGGGAAGATCTCCCACCACTATGATTTATAGGGATGAT |
| | | AAGAGACCAGATGGAGTTCCTGACAGGTTCTCTGGCTCCATTGACAGATCTTC |
| | | CAACTCAGCCCTCCTGACAATCAATAATGTGCAGACTGAAGATGAGGCTGACT |
| | | ACTTCTGTCAGTCTTACAGTAGTGGTATTTATATTTTCGGCGGTGGAACCAAG |
| | | CTCACTGTCCTG |
| 231 | RNF43-86 (VH | EVQLVESGGGLVQPGRSLKLSCAASGFTFTNYYMAWVRQAPTKGLEWVASISP |
| | prot) | SGDTTYYPDSVKGRFTVSRDNAKSTQYLQMDNLRSEDTATYYCARDAYTYYGY |
| | | KGWYFDFWGPGTMVTVSS |
| 232 | RNF43-86 (VL | QFVLTQPNSVSTNLGSAVKLSCKRSTDDIGSNYVNWYQQHEGRSPTTMIYRDD |
| | prot) | KRPDGVPDRFSGSIDRSSNSALLTINNVQTEDEADYFCQSYSSGIYIFGGGTK |
| | | LTVL |
| 233 | RNF43-90 (VH | CAGGTACAGTTGAAGGAGTCAGGACCTGGTCTGGTGCAGCCCTCACAGACCCT |
| | DNA) | GTCCCTCACCTGCACTGTCTCTGGATTCTCACTTACCAGCTACAGTGTACACT |
| | | GGGTTCGCCAACATTCAGGAAACCGTCTGGAATGGATGGGAAGAATGTGGAGT |
| | | GCTGGAGACACATCATTTAATTCAGCGTTCACATCCCGACTGAACATCACCAG |
| | | GGACACCTCCAAGAGCCAAGTTTTCTTAAAAATGAACAGTCTGCAAACTGAAG |
| | | ACACAGGCACTTACTACTGTGTCAGATCCTTCTATAATGCGGGGTATCCTTTT |
| | | GATTACTGGGGCCAGGGAGTCATGGTCACAGTCTCCTCA |
| 234 | RNF43-90 (VL | GACACTGTACTGACCCAGTCTCCTGCTTTGGCTGTGTCTCCAGGAGAGAGGGT |
| | DNA) | TACTATCTCCTGTAGGGCCAGTGATAGTGTCATTAAACTTATGAACTGGTATC |
| | | AACAGAAACCAGGACAGCGACCCAAACTCCTCATCTATCTGGCATCACACCTA |
| | | GCATCTGGGGTCCCTGCCAGGTTCAGTGGCAGTGGGTCTGGGACAGACTTCAC |
| | | CCTCACCATTGATCCTGTGGAGACTGATGACACTGCAACCTATTACTGTCAGC |
| | | AGAGTAGGAATGATCCCACGTTTGGACCTGGGACCAAGCTGGAACTGAAA |
| 235 | RNF43-90 (VH | QVQLKESGPGLVQPSQTLSLTCTVSGFSLTSYSVHWVRQHSGNRLEWMGRMWS |
| | prot) | AGDTSFNSAFTSRLNITRDTSKSQVFLKMNSLQTEDTGTYYCVRSFYNAGYPF |
| | | DYWGQGVMVTVSS |
| 236 | RNF43-90 (VL | DTVLTQSPALAVSPGERVTISCRASDSVIKLMNWYQQKPGQRPKLLIYLASHL |
| | prot) | ASGVPARFSGSGSGTDFTLTIDPVETDDTATYYCQQSRNDPTFGPGTKLELK |
| 237 | ZNRF3-101 (VH | CAGGTGCAACTGAAGGAGTCAGGACCTGGTCTGGTGCAGCCCACACAGACCCT |
| | DNA) | GTCCCTCACCTGCACTGTCTCTGGGTTTTCACTGACCAGCTCTACTGTAAGCT |
| | | GGGTTCGCCAACCTCCAGGAAAGGGTCTGGAGTGGATTGCAGCAATATCAAGT |
| | | GCTGAAAACACATATTATAATTCAGGTCTCAAATCCCGACTGAGCATCAGCAG |
| | | GGACACCTCCAAAAGCCAAGTTTTCTTAAAAATGAACAGTCTGCAAACTGAAG |
| | | ACACAGCCATGTACTTCTGTGCCGGCGGTAGAATACTGGAGACCCTCTTTGAT |
| | | TCCTGGGGCCAAGGAGTCATGGTCACAGTCTCCTCA |
| 238 | ZNRF3-101 (VL | GACATTGTGCTGACCCAGTCTCCTGCTTTGGCTGTGTCTCTAGGGCAGAGGGC |
| | DNA) | CACCATCTCTTGCAAGACCAACCAGAATGTCGATTTTTATGGCAAAACTTATA |
| | | TGCACTGGTACCAACAGAAACGGGGACAACAACCCAAACTCCTCATCTATTTA |
| | | GCATCCAACTTAGCATCTGGGATCCCTGCCAGGTTCAGTGGTAGAGGGTCTGG |
| | | GACAGACTTCACCCTCACCATTGATCCTGTGGCGACTGATGATACTGCAACCT |
| | | ATTACTGTCAGCAGACTAGGAGTCTTCCTCCCACGTTTGGAGCTGGGACCAAG |
| | | CTGGAACTGAAA |
| 239 | ZNRF3-101 (VH | QVQLKESGPGLVQPTQTLSLTCTVSGFSLTSSTVSWVRQPPGKGLEWIAAISS |
| | prot) | AENTYYNSGLKSRLSISRDTSKSQVFLKMNSLQTEDTAMYFCAGGRILETLFD |
| | | SWGQGVMVTVSS |
| 240 | ZNRF3-101 (VL | DIVLTQSPALAVSLGQRATISCKTNQNVDFYGKTYMHWYQQKRGQQPKLLIYL |
| | prot) | ASNLASGIPARFSGRGSGTDFTLTIDPVATDDTATYYCQQTRSLPPTFGAGTK |
| | | LELK |
| 241 | ZNRF3-117 (VH | GAGATACAGCTGCAGGAGTCAGGACCTGGCCTTGTGAAACCTTCACAGTCACT |
| | DNA) | CTCCCTCACCTGTTCTGTCACTGGTTACACCATTACCAGTGATTATGATTGGA |
| | | GCTGGATCCGGAAGTTCCCAGGAAATAAAATGGAGTGGATGGGATACATAAAC |
| | | TACAGTGGTAGCACTAACTACAACCCATCGCTCAAAAGTCGAATCTCCTTTAC |
| | | CAGAGACACATCCAAGAATCAGTTCTTCCTGCAGTTGAACTCTATAACTACTG |
| | | ACGATACAGCCACATATTACTGTGCAAGATATGGTCCATACTACGGGTTTTCC |
| | | GACTGGTACTTTGACTTCTGGGGCCCGGGAACCATGGTCACCGTGTCCTCA |
| 242 | ZNRF3-117 (VL | GATGTCCAGATGACCCAGTCTCCGTCTTATCTTGCTGCGTCTCCTGGAGAAAG |
| | DNA) | TGTTTCCATCAGTTGCAAGGCAAGTCAGAGCATTAGTAATTATTTAGCCTGGT |
| | | ATCAACAGAAACCTGGAAAAACATATAGGCTTCTTATCTACTCTGGGTCAACT |
| | | TTGCAATCTGGAACTCCATCAAGGTTCAGTGGCAGTGGATCTGGTACAGATTT |
| | | CACTCTCACCATCAGAAGCCTGGAGCCTGAAGATTTTGGACTCTATTACTGTC |
| | | AACAGTATTATGAAAAACCGCTCACGTTCGGTTCTGGGACCAAGCTGGAGATC |
| | | AAA |
| 243 | ZNRF3-117 (VH | EIQLQESGPGLVKPSQSLSLTCSVTGYTITSDYDWSWIRKFPGNKMEWMGYIN |
| | prot) | YSGSTNYNPSLKSRISFTRDTSKNQFFLQLNSITTDDTATYYCARYGPYYGES |
| | | DWYFDFWGPGTMVTVSS |
| 244 | ZNRF3-117 (VL | DVQMTQSPSYLAASPGESVSISCKASQSISNYLAWYQQKPGKTYRLLIYSGST |
| | prot) | LQSGTPSRFSGSGSGTDFTLTIRSLEPEDFGLYYCQQYYEKPLTFGSGTKLEI |
| | | K |
| 245 | ZNRF3-128 (VH | GAGGTACAGCTGGTGGAGTCTGGAGGAGGCTTAGTGCAGCCTGGAAAGTCCCT |
| | DNA) | GAAACTCTCCTGTTCAGCCTCTGGATTCACATTCAGTAGCTATGGCATGCACT |
| | | GGATTCGCCAAGCTCCAGGAAAGGGGCTAGATTGGGTTGCATACATTAGTACT |
| | | AACAGCGCTACAGTCTATGCAGACGCTGTGAAGGGCCGGTTCACCATCTCCAG |
| | | AGACAATGCAAAGAACACCCTGTACCTACAACTCAACAGTCTGAAGTCTGAGG |
| | | ACACTGCCATCTATTACTGTGCAAGAAGCGGCTATACCACCTATATGCCCTTT |
| | | ACTTACTGGGGCCAAGGCACTCTGGTCACTGTCTCTTCA |
| 246 | ZNRF3-128 (VL | GATGTTTTGATGACACAAACTCCAGTCTCCCTGCCTGTCAGCCTTGGAGGTCA |
| | DNA) | AGTCTCTATCTCTTGCCGGTCAAGTCAGAGCCTTGTACACAGTGATGGAAACA |
| | | CCTACTTGCATTGGTATCTGCAGAAGCCAGGCCAGTCTCCACAGCTCCTCATC |
| | | TATCGGGTTTCCAACAGATTTTCTGGGGTGTCAGACAGGTTCAGTGGCAGTGG |
| | | GTCAGGGACAGATTTCACCCTCAAGATCAGCAGAGTAGAGCCTGACGACTTGG |
| | | GAATTTATTACTGCTTACAAACTACACATTTTCTCACGTGGACGTTCGGTGGA |
| | | GGCACCAAGCTGGAATTGAAA |
| 247 | ZNRF3-128 (VH | EVQLVESGGGLVQPGKSLKLSCSASGFTFSSYGMHWIRQAPGKGLDWVAYIST |
| | prot) | NSATVYADAVKGRFTISRDNAKNTLYLQLNSLKSEDTAIYYCARSGYTTYMPF |
| | | TYWGQGTLVTVSS |
| 248 | ZNRF3-128 (VL | DVLMTQTPVSLPVSLGGQVSISCRSSQSLVHSDGNTYLHWYLQKPGQSPQLLI |
| | prot) | YRVSNRFSGVSDRFSGSGSGTDFTLKISRVEPDDLGIYYCLQTTHELTWTFGG |
| | | GTKLELK |
| 249 | ZNRF3-131 (VH | GAGGTGCAGGTGGTGGAGTCTGGGGGAGGCCTAGTGCAGCCTGGAAGATCCCT |
| | DNA) | GAAAGTCTCCTGTGCAGCCTCAGGATTCACTTTCAGTAGATATGGCATGGCTT |
| | | GGGTCCGCCAGGCTCCGACGAAGGGTCTGGAGTGGGTCGCATCCATTAGTACT |
| | | GGTGGTGAAAACACTTACTATCGAGACTCCGTGAAGGGCCGATTCACTATCTC |
| | | CAGAGATAATGTAAAAAACACCCTATACCTGCAAATGGACAGTCTGAGGTCTG |
| | | AGGACACGGCCACTTATTATTGTGCAAGACATGGGCGGGTACTAAATTACTTT |
| | | GATTACTGGGGCCAAGGAGTCATGGTCACAGTCTCTTTA |
| 250 | ZNRF3-131 (VL | GACATCCAGATGACACAGTCTCCAGCTTCCCTGTCTGCATCTCTGGGAGAAAC |
| | DNA) | TGTCTCCATCGAATGTCTAACAAGTGAGGACATATACACTTATTTAGCATGGT |
| | | ATCAGCAGAAGCCAGGGAAATCTCCTCACCTCCTGATCTTTGCTGCAAGTAGG |
| | | TTGCAAGATGGGGTCCCATCACGGTTCAGTGGCAGTGGATCTGGCACACAGTA |
| | | TTCTCTCAAGATCAGCGGCATGCAACCTGAAGATGAAGGGGATTATTTCTGTC |
| | | TACAGGGTTCCAAGTTTCCGTACACGTTTGGAGCTGGGACCAAGCTGGACCTG |
| | | AAA |
| 251 | ZNRF3-131 (VH | EVQVVESGGGLVQPGRSLKVSCAASGFTFSRYGMAWVRQAPTKGLEWVASIST |
| | prot) | GGENTYYRDSVKGRFTISRDNVKNTLYLQMDSLRSEDTATYYCARHGRVLNYF |
| | | DYWGQGVMVTVSL |
| 252 | ZNRF3-131 (VL | DIQMTQSPASLSASLGETVSIECLTSEDIYTYLAWYQQKPGKSPHLLIFAASR |
| | prot) | LQDGVPSRFSGSGSGTQYSLKISGMQPEDEGDYFCLQGSKFPYTFGAGTKLDL |
| | | K |
| 253 | ZNRF3-163 (VH | GAGGTGCAGTTGGAGGAGTCTGGGGGAGGCTTAGTGCAGCCTGGAAGGTCCAT |
| | DNA) | GAAATTCTCCTGTGCAGCCTCAGGATTCACTTTCAGTAACTATTACATGGCCT |
| | | GGGTCCGCCAGGCTCCATCGAAGGGTCTGGAGTGGGTCGCATCCATTAGTACT |
| | | GGTGGAGGTGATGTTTACTATCGAGACTCCGTGAAGGGCCGATTCACGATCTC |
| | | CAGAGATAATGCAAAAAGCACCCTATACCTGCAAATGGACAGTCTGAGGTCTG |
| | | AGGACACGGCCACTTATTACTGTGCAAGACATTCGGGGGGTAGAATTCGGGCC |
| | | CGGGGTCACTACTTTGATTACTGGGGCCAAGGAGTCATGGTCACAGTCTCCTC |
| | | A |
| 254 | ZNRF3-163 (VL | GACATCCAGATGACACAGTCTCCAGCTTCCCTGTCTGCATCTCTTGGAGAAAC |
| | DNA) | TGTCTCCATCGAATGTCTAGCAAGTGAGGACATTTCCAATGATTTAGCGTGGT |
| | | ATCAGCAGAAGTCAGGGAAATCTCCTCAGGTCCTGATCTATGGTGTAAGCAGG |
| | | TTGCAAGACGGGGTCCCATCACGGTTCAGTGGCAGTGGATCTGGCACACGGTA |
| | | TTCTCTCAAGATCAGCGGCATGCAACCTGAAGATGAAGCAGATTATTTCTGTC |
| | | AACAGAGTTACAAGTATCCGTACACGTTTGGAGCTGGGACCAAGCTGGAACTG |
| | | AAA |
| 255 | ZNRF3-163 (VH | EVQLEESGGGLVQPGRSMKFSCAASGFTFSNYYMAWVRQAPSKGLEWVASIST |
| | prot) | GGGDVYYRDSVKGRFTISRDNAKSTLYLQMDSLRSEDTATYYCARHSGGRIRA |
| | | RGHYFDYWGQGVMVTVSS |
| 256 | ZNRF3-163 (VL | DIQMTQSPASLSASLGETVSIECLASEDISNDLAWYQQKSGKSPQVLIYGVSR |
| | prot) | LQDGVPSRFSGSGSGTRYSLKISGMQPEDEADYFCQQSYKYPYTFGAGTKLEL |
| | | K |
| 257 | ZNRF3-17 (VH | GAGGTGCAGCTGGTGGAGTCTGGGGGAGGCTTAGTGCAGCCTGGAGGGTCCCT |
| | DNA) | GAAACTCTCCTGTGCAGCCTCAGGATTCACTTTCAGTAATTATGACATGGCCT |
| | | GGGTCCGCCAGGCTCCAACGAAGGGTCTGGAGTGGGTCGCATCCATTAGTACT |
| | | GGTGGTGGTAAAACTTACTATCGAGACTCCGTGAAGGGCCGACTCACTATCTC |
| | | CAGAGATAATGCAAAAAACACCCAATACCTGCAAATGGACAGTCTGAGGTCTG |
| | | AGGACACGGCCACTTATTACTGTGCAAGACTCGGTCCGGCATACTCCGGGGAG |
| | | TGGTTTGCTTACTGGGGCCAAGGCACTCTGGTCACTGTCTCTTCA |
| 258 | ZNRF3-17 (VL | GACACTGTGCTGACCCAGTCTCCTGCTTTGGCTGTGTCTCCAGGAGAGAGGGT |
| | DNA) | TACCATCTCCTGTGGGGCCAGTGAAGGTGTCAATACACGTATGCACTGGTACC |
| | | AACAGAGATCAGGACAACAACCCAAACTCCTCATCTACGGTTCCTCCAACCTA |
| | | GATTCTGGAGTCCCTGACAGGTTCAGTGGCAGTGGGTCTGGGACAGACTTTAC |
| | | CCTCACCATCGATCCTGTGGAGGCTAAAGATGCTGCAACCTATTTCTGTCAGC |
| | | AGAGTTGGAATGTTCCGAACACGTTTGGAGCTGGGACCAAACTGGAACTGAAA |
| 259 | ZNRF3-17 (VH | EVQLVESGGGLVQPGGSLKLSCAASGFTFSNYDMAWVRQAPTKGLEWVASIST |
| | prot) | GGGKTYYRDSVKGRLTISRDNAKNTQYLQMDSLRSEDTATYYCARLGPAYSGE |
| | | WFAYWGQGTLVTVSS |
| 260 | ZNRF3-17 (VL | DTVLTQSPALAVSPGERVTISCGASEGVNTRMHWYQQRSGQQPKLLIYGSSNL |
| | prot) | DSGVPDRFSGSGSGTDFTLTIDPVEAKDAATYFCQQSWNVPNTFGAGTKLELK |
| 261 | ZNRF3-170 (VH | GAGGTGCAGCTGGTGGAGTCTGGGGGAGGCTTAGTGCAGCCTGGAAGGACCCT |
| | DNA) | GAAACTCTCCTGTGCAGCCTCAGGATTCACTTTCAGTGACTATTACATGGCCT |
| | | GGGTCCGCCAGGCTCCAAAGAAGGGTCTGGAGTGGGTCGCATCCATTAGTTCT |
| | | GGGGGTTTTAGTATTTATTATGGAGACTCCGTGAAGGGCCGATTCACTATCTC |
| | | CAGAGATAATGCAAAAACCACCCTATACCTGCAAATGAACAGTCTGAGGTCTG |
| | | AGGACACGGCCACTTATTATTGTGCAAGACATACGATTAGTACGGCCTTTTTT |
| | | GATTACTGGGGCCAAGGAGTCATGGTCACAGTCTCCTCA |
| 262 | ZNRF3-170 (VL | GACACTGTGCTGACCCAGTCTCCTGCTTTGGCTGTGTCTCCAGGAGAGAGGGT |
| | DNA) | TACCATCTCCTGTAAGGCCAATGAAAATGTCAGTACACGTATGCACTGGTACC |
| | | AACAGAGACCAGGACAGCGACCCAAACTCCTCATCTATAAAGCATCAAACCTA |
| | | GGATCTGGGGTCCCTGCCAGGTTCAGTGGCAGTGGGTCTGGGACAGACTTCAC |
| | | CCTCACCATTGATCCTGTGGAGGCTGATGACACTGCAACCTATTTCTGTCAGC |
| | | AGAGTTGGAATGGTCCTCCGTGGACGTTCGGTGGAGGCACCAAGCTGGAGTTG |
| | | AAA |
| 263 | ZNRF3-170 (VH | EVQLVESGGGLVQPGRTLKLSCAASGFTFSDYYMAWVRQAPKKGLEWVASISS |
| | prot) | GGFSIYYGDSVKGRFTISRDNAKTTLYLQMNSLRSEDTATYYCARHTISTAFF |
| | | DYWGQGVMVTVSS |
| 264 | ZNRF3-170 (VL | DTVLTQSPALAVSPGERVTISCKANENVSTRMHWYQQRPGQRPKLLIYKASNL |
| | prot) | GSGVPARFSGSGSGTDFTLTIDPVEADDTATYFCQQSWNGPPWTFGGGTKLEL |
| | | K |
| 265 | ZNRF3-171 (VH | CAGGTCCAGTTGCACCAATCTGGAACTGAACTGGCCAGACCTGGGACTTCAGT |
| | DNA) | GAAGCTGTCCTGCAAGGCTTCTGGCTATACCTTCAGCAGGTTTTATATTAACT |
| | | GGGTGAAGAAGAGGCCTGGACAGGGCCTTGAGTGGATCGGATATATTGATCCT |
| | | GGAAATGGTGGTACTTTCCACAGTGAGAAGTTCAAGGACAAAGCCTCACTTAC |
| | | TGCAGACACATCTTCCAGCACAGCCTACATGGTACTGGGCAGCCTGGCACCTG |
| | | AAGACTCTGCATTCTATTTCTGTGTAAGGGGTTACTATAGCAGCTATGATTGG |
| | | TTTGCTTACTGGGGCCAAGGCACTCTGGTCACTGTCTCTTCA |
| 266 | ZNRF3-171 (VL | GACATCCAGTTGACCCAGTCTCCATCCTCCATGTCTGCATCTCTGGGAGACAG |
| | DNA) | AGTCAGTCTTACTTGCCAGTCAAGCCAGGGCATTGGAAATTATTTAAGCTGGT |
| | | ACCAGCATAAACCAGGGGAACCTCCTAAGCCTATACTTTATTATGCAACCAAC |
| | | TTGGCAGATGGGGTCCCATCAAGATTCAGTGGCAGTAGGTCTGGGTCAGATTA |
| | | TTCTCTCACCATCAGCAGCCTGGAGTCTGAAGATACAGCAATCTATTACTGTC |
| | | TACAGTATGATGAGTACCCTTACACGTTTGGACCTGGGACCAAGCTGGAACTG |
| | | AGA |
| 267 | ZNRF3-171 (VH | QVQLHQSGTELARPGTSVKLSCKASGYTFSRFYINWVKKRPGQGLEWIGYIDP |
| | prot) | GNGGTFHSEKFKDKASLTADTSSSTAYMVLGSLAPEDSAFYFCVRGYYSSYDW |
| | | FAYWGQGTLVTVSS |
| 268 | ZNRF3-171 (VL | DIQLTQSPSSMSASLGDRVSLTCQSSQGIGNYLSWYQHKPGEPPKPILYYATN |
| | prot) | LADGVPSRFSGSRSGSDYSLTISSLESEDTAIYYCLQYDEYPYTFGPGTKLEL |
| | | R |
| 269 | ZNRF3-172 (VH | GAGGTGCAGCTGGCGGAGTCTGGGGGAGGCTTAGAGCAGCCTGGAAGGTCCCT |
| | DNA) | GAAACTCTCCTGTGCAGCCTCAGGATTCACTTTCAGTAACTATGACATGGCCT |
| | | GGGTCCGCCAGGCTCCAACGAAGGGTCTGGAGTGGGTCGCATCCATTATTGCT |
| | | AGTGGTGGTACTACTTATTATCGAGACTCCGTGAAGGGCCGATTCACTGTCTC |
| | | CAGAGATAATGCAAAAAGCACCCTATACCTACAAATGGACAGTCTGAGGTCTG |
| | | AGGACACGGCCACTTATTACTGTGCAAGACATGGGGTCGGTAGCTACGATTGG |
| | | TTTGCTGACTGGGGCCAAGGCACTCTGGTCACTGTCTCTTCA |
| 270 | ZNRF3-172 (VL | GACATTGTCTTGACCCAGTCTCCTGCTTTGGCTGTGTCTCTAGGGCAGAGGGC |
| | DNA) | CACAATCTCCTGTAGGGCCAGCCAAAGTGTCACTATATCTGGATTTAATCTTA |
| | | TGCACTGGTACCAACAGAAACCAGGACAACAACCCAAGCTCCTCATCTATCGT |
| | | GCATCCAACCTAGCATTTGGGATCCCTGCCAGGTTCAGTGGCAGTGGGTCTGG |
| | | GACAGACTTCACCCTCACCATCAATCCTGTGCAGGCTGATGATATTACAACCT |
| | | ATTACTGTCAGCAAAGTAGGAAGTCTCGGACGTTCGGTGGAGGCACCAAACTG |
| | | GAATTGAAA |
| 271 | ZNRF3-172 (VH | EVQLAESGGGLEQPGRSLKLSCAASGFTFSNYDMAWVRQAPTKGLEWVASIIA |
| | prot) | SGGTTYYRDSVKGRFTVSRDNAKSTLYLQMDSLRSEDTATYYCARHGVGSYDW |
| | | FADWGQGTLVTVSS |
| 272 | ZNRF3-172 (VL | DIVLTQSPALAVSLGQRATISCRASQSVTISGFNLMHWYQQKPGQQPKLLIYR |
| | prot) | ASNLAFGIPARFSGSGSGTDFTLTINPVQADDITTYYCQQSRKSRTFGGGTKL |
| | | ELK |
| 273 | ZNRF3-179 (VH | GAGGTGCAGCTGGCGGAGTCTGGGGGAGGCTTAGAGCAGCCTGGAAGGTCCCT |
| | DNA) | GAAACTCTCCTGTGCAGCCTCAGGATTCACTTTCAGTAACTATGACATGGCCT |
| | | GGGTCCGCCAGGCTCCAACGAAGGGTCTGGAGTGGGTCGCATCCATTATTACT |
| | | AGTGGTGATACTACTTATTATCGAGACTCCGTGAAGGGCCGATTCACTGTCTC |
| | | CAGAGATAATGCAAAAAGCACCCTATACCTGCAAATGGACAGTCTGAGGTCTG |
| | | AGGACACGGCCACTTATCACTGTGCAAGACATGGGGTCGGTAGCTACGATTGG |
| | | TTTGCTGACTGGGGCCAAGGCACTCTGGTCACTGTCTCTTCA |
| 274 | ZNRF3-179 (VL | GACATTGTCTTGACCCAGTCTCCTGCTTTGGCTGTGTCTCTAGGGCAGAGGGC |
| | DNA) | CACAATCTCCTGTAGGGCCAGCCAAAGTGTCACTATATCTGGATATAATCTTA |
| | | TGCACTGGTACCAACAGAAACCAGGACAACAACCCAAACTCCTCATCTATCGT |
| | | GCATCCAACCTAGCATTTGGGATCCCTGCCAGGTTCAGTGGCAGTGGGTCTGG |
| | | GACAGACTTCACCCTCACCATCAATCCTGTGCAGGCTGATGATATTACAACCT |
| | | ATTACTGTCAGCAGAGTAGGAAGTCTCGGACGTTCGGTGGAGGCACCAAACTG |
| | | GAATTGAAA |
| 275 | ZNRF3-179 (VH | EVQLAESGGGLEQPGRSLKLSCAASGFTFSNYDMAWVRQAPTKGLEWVASIIT |
| | prot) | SGDTTYYRDSVKGRFTVSRDNAKSTLYLQMDSLRSEDTATYHCARHGVGSYDW |
| | | FADWGQGTLVTVSS |
| 276 | ZNRF3-179 (VL | DIVLTQSPALAVSLGQRATISCRASQSVTISGYNLMHWYQQKPGQQPKLLIYR |
| | prot) | ASNLAFGIPARFSGSGSGTDFTLTINPVQADDITTYYCQQSRKSRTFGGGTKL |
| | | ELK |
| 277 | ZNRF3-182 (VH | GAGGTGCAGCTGGCGGAGTCTGGGGGAGGCTTAGAGCAGCCTGGAAGGTCCCT |
| | DNA) | GAAACTCTCCTGTGCAGCCTCAGGATTCACTTTCAGTAACTATGACATGGCCT |
| | | GGGTCCGCCAGGCTCCAACGAAGGGTCTGGAGTGGGTCGCATCCATTATTAAA |
| | | AGTGGTGATACTTCTTATTATCGAGACTCCGTGAAGGGCCGATTCACTGTCTC |
| | | CAGAGATAATGCAAAAAGCACCCTATACCTGCAAATGGACAGTCTGAGGTCTG |
| | | AGGACACGGCCACTTATTACTGTGCAAGACATGGGGTCGGTAGCTACGATTGG |
| | | TTTGCTGACTGGGGCCAAGGCACTCTGGTCACTGTCTCTTCA |
| 278 | ZNRF3-182 (VL | GACATTGTCTTGACCCAGTCTCCTGCTTTGGCTGTGTCTCTAGGGCAGAGGGC |
| | DNA) | CACAATCTCCTGTAGGGCCAGCCAAAGTGTCACTATATCTGGATTTAATCTTA |
| | | TGCACTGGTACCAACAGAAACCAGGACAACAACCCAAACTCCTCATCTATCGT |
| | | GCATCCAACCTAGCATTTGGGATCCCTGCCAGGTTCAGTGGCAGTGGGTCTGG |
| | | GACAGACTTCACCCTCACCATCAATCCTGTGCAGGCTGATGATTTTACAACCT |
| | | ATTACTGTCAGCAGAGTAGGAAGTCTCGGACGTTCGGTGGAGGCACCAAACTG |
| | | GAATTGAAA |
| 279 | ZNRF3-182 (VH | EVQLAESGGGLEQPGRSLKLSCAASGFTFSNYDMAWVRQAPTKGLEWVASIIK |
| | prot) | SGDTSYYRDSVKGRFTVSRDNAKSTLYLQMDSLRSEDTATYYCARHGVGSYDW |
| | | FADWGQGTLVTVSS |
| 280 | ZNRF3-182 (VL | DIVLTQSPALAVSLGQRATISCRASQSVTISGFNLMHWYQQKPGQQPKLLIYR |
| | prot) | ASNLAFGIPARFSGSGSGTDFTLTINPVQADDFTTYYCQQSRKSRTFGGGTKL |
| | | ELK |
| 281 | ZNRF3-195 (VH | GAGGTACAGCTGGTGGAGTCTGGAGGAGGCTTAGTGCAGCCTGGAAACTCCCT |
| | DNA) | GAACCTCTCCTGTTCAGCCTCTGGATTCCCATTCAGTACCTATGGCATGCACT |
| | | GGATTCGCCAAGCTCCAGGAAAGGGGCTAGATTGGGTTGCATACATTTCTTAT |
| | | AACAGCGGTACAGTCTATGCAGACGCTGTGAAGGGCCGGTTCACCATCTCCAG |
| | | AGACAATGCAAAGAACACCCTGTACCTGCAACTCAACAGTCTGAAGTCTGAAG |
| | | ACACTGCCATCTATTACTGTGCAAGAGGTTACTATGATGGTTATTATCGTTAC |
| | | TGGGGCCAAGGAGTCATGGTCACAGTCTCCTCG |
| 282 | ZNRF3-195 (VL | GACATTGTGATGACCCAGTCTCCATTCTCCCTGGCTGTGTCAGAAGGAGAGAT |
| | DNA) | GGTCACTATAAACTGCAAGTCCAGTCAGAGTCTTTTATCCAGTGGAAACCAAA |
| | | AGAACTACTTGGCCTGGTACCAGCAGAGACCAGGGCAGTCTCCTAAACTACTG |
| | | ATCTACCATGCATCCACTAGGCAATCAGGGGTCCCTGATCGCTTCATTGGCAG |
| | | TGGATCTGGGACAGACTTCACTCTGACCATCAGCGATGTGCAGGCTGAAGACC |
| | | TGGCAGATTATTACTGCCTACAACATTACAGCTCTCCCACGTTCGGCTCAGGG |
| | | ACGAAGTTGGAGATAAAA |
| 283 | ZNRF3-195 (VH | EVQLVESGGGLVQPGNSLNLSCSASGFPFSTYGMHWIRQAPGKGLDWVAYISY |
| | prot) | NSGTVYADAVKGRFTISRDNAKNTLYLQLNSLKSEDTAIYYCARGYYDGYYRY |
| | | WGQGVMVTVSS |
| 284 | ZNRF3-195 (VL | DIVMTQSPFSLAVSEGEMVTINCKSSQSLLSSGNQKNYLAWYQQRPGQSPKLL |
| | prot) | IYHASTRQSGVPDRFIGSGSGTDFTLTISDVQAEDLADYYCLQHYSSPTFGSG |
| | | TKLEIK |
| 285 | ZNRF3-219 (VH | GAGGTGCAGCTGGTGGAGTCTGGGGGAGGCCTAGTGCAGCCTGGAAGGTCCCT |
| | DNA) | GAAAGTCTCCTGTGCAGCCTCAGGATTCACTTTCAGTACCTTTGCAATGGCCT |
| | | GGGTGCGCCAGGCTCCAAAGAAGGGTCTGGAGTGGGTTGCAACCATTACTCCT |
| | | GGTGGTAGTAACACTTACTATCCAGACTCCGTGAAAGGCCGATTCACTATCTC |
| | | CAGAGATAATGCAAAAAGCACCCTATACCTGCAAATGGACAGTCTGAGGTCTG |
| | | AGGACACGGCCAATTATTACTGTGCAAAGTGCGGCTACCCGGGTATAACGTCT |
| | | TTTGATTACTGGGGCCAAGGAGTCATGGTCACAGTCTCCTCA |
| 286 | ZNRF3-219 (VL | GACATCCAGATGACCCAGTCTCCTTCATTCCTGTCTGCATCTGTGGGAGACAG |
| | DNA) | AGTCTCTATCAACTGCAAAGCAAGTCAGAATATTGGCAAGTACTTAAACTGGT |
| | | ATCAGCAAGAGCTTGGAGAAGCTCCCAAACGCCTGATATATAATACAAACAAT |
| | | TTGCAAACAGGCATCCCATCAAGGTTCAGTGGCAGTGGATCTGGTACAGATTA |
| | | CACACTCACCATCAGCGGCCTGCAGCCTGAAGATTTTGCCACATATTTCTGCT |
| | | TGCAGCATAATGGTTTTCCGCTCACGTTCGGTTCTGGGACCAAGCTGGAGATC |
| | | AAA |
| 287 | ZNRF3-219 (VH | EVQLVESGGGLVQPGRSLKVSCAASGFTFSTFAMAWVRQAPKKGLEWVATITP |
| | prot) | GGSNTYYPDSVKGRFTISRDNAKSTLYLQMDSLRSEDTANYYCAKCGYPGITS |
| | | FDYWGQGVMVTVSS |
| 288 | ZNRF3-219 (VL | DIQMTQSPSFLSASVGDRVSINCKASQNIGKYLNWYQQELGEAPKRLIYNTNN |
| | prot) | LQTGIPSRFSGSGSGTDYTLTISGLQPEDFATYFCLQHNGFPLTFGSGTKLEI |
| | | K |
| 289 | ZNRF3-222 (VH | GAAGTGCAGCTGGTGGAGTCTGGTGGAGGCCTAGTGCAGCCGGGAAGGTCCCT |
| | DNA) | GAAACTCTCCTGTGCAGTCTCAGGATTCACCTTCAGTAACTATGGCATGGCCT |
| | | GGGTCCGCCAGACTCCAACGAAGGGGCTGGAGTGGGTCGCAACCATTACTTAT |
| | | GATGGTAGTACCACTTACTATCGAGACTCCGTGAAGGGCCGATTCACTATCTC |
| | | CAGAGATAATGCAAAGAGCGCCCTATCCCTGCAAATGGACAGTCTGAGGTCTG |
| | | AGGACACGGCCACTTATCACTGTGCAAGGCAGGGTTTGGGGTACCCGTTTGTC |
| | | TACTGGGGTCAAGGCACTCTGGTCACTGTCTCTTCC |
| 290 | ZNRF3-222 (VL | GATGTTGTGATGACACAAACTCCAGTCTCCCTGCCTGTCAGCCTTGGAGGTCA |
| | DNA) | AGCCTCTATCTCTTGCCGGACAAGTCAGAGCCTGGTACATAGTAATGGAAACA |
| | | CCTACTTGCATTGGTACCTGCAGAAGCCAGGCCAGTCTCCACAGCTCCTCATC |
| | | TATCGGGTTTCCAACAGATTCTCTGGGGTGCCAGACAGGTTCAGTGGCAGTGG |
| | | GTCAGGGACAGATTTCACCCTCAAGATCAGTAGAATAGAGCCTGAGGACTTGG |
| | | GAGATTATTACTGCTTACAAAGTACACATTTTCCGTACACGTTTGGAGCTGGG |
| | | ACCAAGCTGGAACTGAAA |
| 291 | ZNRF3-222 (VH | EVQLVESGGGLVQPGRSLKLSCAVSGFTFSNYGMAWVRQTPTKGLEWVATITY |
| | prot) | DGSTTYYRDSVKGRFTISRDNAKSALSLQMDSLRSEDTATYHCARQGLGYPFV |
| | | YWGQGTLVTVSS |
| 292 | ZNRF3-222 (VL | DVVMTQTPVSLPVSLGGQASISCRTSQSLVHSNGNTYLHWYLQKPGQSPQLLI |
| | prot) | YRVSNRFSGVPDRFSGSGSGTDFTLKISRIEPEDLGDYYCLQSTHFPYTFGAG |
| | | TKLELK |
| 293 | ZNRF3-223 (VH | GAGGTACAGCTGGTGGAGTCTGGAGGAGGCTTAGTGCAGCCTGGAGAGTCCCT |
| | DNA) | GAAACTCTCCTGTTCAGCCTCTGGATTCACATTCAGTAACTATGGCATGCACT |
| | | GGATACGCCAAGTTCCAGGAAAGGGGCTAGATTGGGTTGCATACATTACTAAT |
| | | AGCGGCGGTTCACTCTATGCAGCCGCTGTGAAGGGCCGGTTCACCATCTCCAG |
| | | AGACAATGCAAAGAACACCCTGTACCTGGAACTCACCAGTCTGAAGTCTGAGG |
| | | ACACTGCCATCTATCACTGTGCAAGAGGTTATAGTTACCCGGGACCTTACTGG |
| | | GGCCGAGGCACTCTGGTCACGGTCTCTTCA |
| 294 | ZNRF3-223 (VL | GATATTCAAGTGACTCAATCTCCATCCTCCCTCTTGGCATCTCTAGGAGAGAG |
| | DNA) | AGTCACTATCACATGCCAGACAGGTCAGAGCATTAACAATTACCTAAACTGGT |
| | | ATCAGCAGAGGCCAGGACAAGCTCCTCTACTCTTGATCTATTTTGCAACCAGT |
| | | TTGCAGACTGGCGTGCCATCAAGGTTCAGTGGCCAATATTCTGGGAGAAGTTT |
| | | CACTCTAACCATCACTAGCCTGGAGCCAGAAGATATTGCAAATTATTTTTGTC |
| | | TGCAACATTACAGTCCTCCGTACACGTTTGGAGCTGGGACCAAGCTGGAACTG |
| | | AGA |
| 295 | ZNRF3-223 (VH | EVQLVESGGGLVQPGESLKLSCSASGFTFSNYGMHWIRQVPGKGLDWVAYITN |
| | prot) | SGGSLYAAAVKGRFTISRDNAKNTLYLELTSLKSEDTAIYHCARGYSYPGPYW |
| | | GRGTLVTVSS |
| 296 | ZNRF3-223 (VL | DIQVTQSPSSLLASLGERVTITCQTGQSINNYLNWYQQRPGQAPLLLIYFATS |
| | prot) | LQTGVPSRFSGQYSGRSFTLTITSLEPEDIANYFCLQHYSPPYTFGAGTKLEL |
| | | R |
| 297 | ZNRF3-231 (VH | GAGGTACAGCTGGTGGAGTCTGGAGGAGGCTTAGTGCAGCCTGGAGAGTCCCT |
| | DNA) | GAAACTCTCCTGTTCAGCCTCTGGATTCACATTCAGTAACTATGGAATGCACT |
| | | GGATACGCCAAGTTCCAGGAAAGGGACTAGATTGGGTTGCATACATTAGTAAT |
| | | AGCGGCGGTTCACTCTATGCAGACGCTGTGAAGGGCCGGTTCACCATCTCCAG |
| | | AGACAATGCAAAGAACACCCTATACCTGGAACTCAACAGTCTGAAGTCTGAGG |
| | | ACACTGCCATCTATCACTGTGCAAGAGGTTATAGTTACCCGGGACCTTACTGG |
| | | GGCCAAGGCACTCTGGTCACGGTCTCTTCA |
| 298 | ZNRF3-231 (VL | GATATTCAAGTGACTCAATCTCCATCCTCCCTCTTGGCATCTCTAGGAGAGAG |
| | DNA) | AGTCACTATCACATGCCAGACAGGTCAGAGTATTAACAATTACCTGAACTGGT |
| | | ATCAGCAGAGGCCAGGACAAGCTCCTCTACTCTTGATCTATTTTGCAACCAGT |
| | | TTGCAGACTGGCGTGCCATCAAGGTTCAGTGGCCAATATTCTGGGAGAAGTTT |
| | | CACTCTAACCATCACTAGCCTGGAGCCAGAAGATATTGCAAATTATTTTTGTC |
| | | TGCAACATTACAGTCCTCCGTACACGTTTGGAGCTGGGACCAAGCTGGAACTG |
| | | AGA |
| 299 | ZNRF3-231 (VH | EVQLVESGGGLVQPGESLKLSCSASGFTFSNYGMHWIRQVPGKGLDWVAYISN |
| | prot) | SGGSLYADAVKGRFTISRDNAKNTLYLELNSLKSEDTAIYHCARGYSYPGPYW |
| | | GQGTLVTVSS |
| 300 | ZNRF3-231 (VL | DIQVTQSPSSLLASLGERVTITCQTGQSINNYLNWYQQRPGQAPLLLIYFATS |
| | prot) | LQTGVPSRFSGQYSGRSFTLTITSLEPEDIANYFCLQHYSPPYTFGAGTKLEL |
| | | R |
| 301 | ZNRF3-237 (VH | CAGATCCAGTTGGTGCAGTCTGGACCTGAGCTGAAGAAGCCTGGAGAGTCAGT |
| | DNA) | GAAGATCTCCTGCAAGGCTTCTGGGTATACCTTCACAGACTATGCAATGCACT |
| | | GGGTGAAACAGGTTCCAGGAAAGGGCTTGAAGTGGATGGGCTGGATCAACACC |
| | | TATACTGGGAAGCCAACATATGCTGATGACTTCAAAGGACGGTTTGTCTTCTC |
| | | TTTGGAAGCCTCTGCCAGCACTGTAAATTTGCAGATCAGCAACCTCAAAAATG |
| | | AGGACACGGCTAATGTTTTCTGTGCAAGAGGGGGGTTATTATCACGTGGTTGG |
| | | TTTGCTTACTGGGGCCAAGGCACTCTGGTCACTGTCTCTTCA |
| 302 | ZNRF3-237 (VL | GATATTGTGATGACACAAACTCCAGTCTCCCTGTCTGTCAGCCTTGGAGGTCA |
| | DNA) | AGTCTCTATCTCTTGCCGGTCTAGTCAGAGCTTTGTTCATAGTGATGGAAATA |
| | | GCTATTTGAGTTGGTACCTGCAGAAGCCCGGCCAATCTCCACAGCTCCTCATT |
| | | TATGAGGTTTCCAACCGATTATCTGGGGTCCCAGATAGGTTCAGTGGTAGTGG |
| | | TTCAGGGACAGATTTCACCCTCAAGATCAGCAGAGTAGAGCATGATGATTTGG |
| | | GAGTTTATTACTGCGGCCAAGCTTCAAAAATTCCGCTCACGTTCGGTTCTGGG |
| | | ACCAAGCTGGAGATCAAA |
| 303 | ZNRF3-237 (VH | QIQLVQSGPELKKPGESVKISCKASGYTFTDYAMHWVKQVPGKGLKWMGWINT |
| | prot) | YTGKPTYADDFKGRFVFSLEASASTVNLQISNLKNEDTANVFCARGGLLSRGW |
| | | FAYWGQGTLVTVSS |
| 304 | ZNRF3-237 (VL | DIVMTQTPVSLSVSLGGQVSISCRSSQSFVHSDGNSYLSWYLQKPGQSPQLLI |
| | prot) | YEVSNRLSGVPDRFSGSGSGTDFTLKISRVEHDDLGVYYCGQASKIPLTFGSG |
| | | TKLEIK |
| 305 | ZNRF3-244 (VH | GAGGTACAGCTGGTGGAGTCTGGAGGAGGCTTAGTGCAGCCTGGAAAGTCCCT |
| | DNA) | GAAACTCTCCTGTTCAGCCTCTGGATTCACATTCAGTAGCTATGGCATGCACT |
| | | GGATACGCCAAACTCCAGGAAAGGGACTAGATTGGGTTGCATACATTAGTACT |
| | | AACAGCGGTACAGTCTATGCAGACGCTGTGAAGGGCCGGTTCACCATCTCCAG |
| | | AGACAATGCAAAGAACACCCTGTACCTGCAACTCAACAGTCTGCAGTCTGAAG |
| | | ACACTGCCATCTATTACTGTGCAAGAGGTTATAACTACCCGGGAGCTTACTGG |
| | | GGCCAAGGCACTCTGGTCACTGTCTCTTCA |
| 306 | ZNRF3-244 (VL | GATATTCAAGTGACTCAATCTCCATCCTCCCTCTTGGCATCTCTAGGAGAGAG |
| | DNA) | AGTCACTATCACATGCCAGACAAGTCAGAGCATTAGCAATAACCTAAACTGGT |
| | | ATCAGCAGAAACCAGGACAAGCTCCTATGCTCTTGATCTATTATGCAACCAGT |
| | | TTGCAGACTGGCATGCCATCAAGGTTCAGTGGCCAATATTCTGGGAGAAGTTT |
| | | CACTCTAACCATCACTAGCCTGGAGCCAGAAGATATTGCAAATTATTTTTGTC |
| | | TGCAACATTACAATCCTCCGTACACGTTTGGAGCTGGGACCAAGCTGGAACTG |
| | | AAA |
| 307 | ZNRF3-244 (VH | EVQLVESGGGLVQPGKSLKLSCSASGFTFSSYGMHWIRQTPGKGLDWVAYIST |
| | prot) | NSGTVYADAVKGRFTISRDNAKNTLYLQLNSLQSEDTAIYYCARGYNYPGAYW |
| | | GQGTLVTVSS |
| 308 | ZNRF3-244 (VL | DIQVTQSPSSLLASLGERVTITCQTSQSISNNLNWYQQKPGQAPMLLIYYATS |
| | prot) | LQTGMPSRFSGQYSGRSFTLTITSLEPEDIANYFCLQHYNPPYTFGAGTKLEL |
| | | K |
| 309 | ZNRF3-247 (VH | GAGGTGCAGCTGGTGGGGTCTGGTGAAGGCTTAGTACAGCCTGGAAGTTCCAT |
| | DNA) | GAAACTCTCATGTGTAGCCTCTGGATTCACTTTCAGTAACTCTGGCATGAACT |
| | | GGATTCGACAGGCTCCAAAGAAGGGGCTGGAATGGATAGCACTGATTTATTAT |
| | | ACTAACACATACTATGCAGACTCTGTGAAGGGTCGATTCACCATCTCCAGAGA |
| | | CAATTCTAAGAACACCCTGTACCTGGAAATGAACAGTCTGAGATCTGAGGACA |
| | | CAGCCATGTATTACTGTGCAAAAAGGGGTCTAACTGGGAGCGTTGATTACTGG |
| | | GGCCAAGGAGTCATGGTCACAGTCTCCTCA |
| 310 | ZNRF3-247 (VL | GACATCCAGATGACCCAGTCTCCTTCATTCCTGTCTGCATCTGTGGGAGACAG |
| | DNA) | AGTCACTATCAACTGCAAAGCAAGTCAGAATATTAACAGGTACTTAAACTGGT |
| | | ATCAGCAGAAGCTTGGAGAAGCTCCCAAACGCCTAATATATGATACAAGCAAT |
| | | TTGCAAACGGGCATTCCATCAAGGTTCAGTGGCAGTGGGTCTGGTACAGATTT |
| | | CACACTCACCATCAGCAGCCTGCAGCCTGAAGATTTTGCCACATATTTCTGCT |
| | | TGCAGCATGATAGTTTTCCCAACACGTTTGGAACTGGGACCAAGCTGGAGCTG |
| | | AAA |
| 311 | ZNRF3-247 (VH | EVQLVGSGEGLVQPGSSMKLSCVASGFTFSNSGMNWIRQAPKKGLEWIALIYY |
| | prot) | TNTYYADSVKGRFTISRDNSKNTLYLEMNSLRSEDTAMYYCAKRGLTGSVDYW |
| | | GQGVMVTVSS |
| 312 | ZNRF3-247 (VL | DIQMTQSPSFLSASVGDRVTINCKASQNINRYLNWYQQKLGEAPKRLIYDTSN |
| | prot) | LQTGIPSRFSGSGSGTDFTLTISSLQPEDFATYFCLQHDSFPNTFGTGTKLEL |
| | | K |
| 313 | ZNRF3-253 (VH | GAGGTGCAGCTTGTAGAGTCTGGGGGCGGTTTAGTGCAGCCTGAAAGGTCCAT |
| | DNA) | GAAACTCTCCTGTGCAGCCTCAGGATTCACTTTCAGTAACTATGGCATGGCCT |
| | | GGGTCCGCCAGGCTCCAACGAAGGGGCTGGAGTGGGTTGCAACCATTAATTCT |
| | | GATGGTAGTGGCACTTACTATCGAGGCTCCGTGAAGGGCCGATTCACTATCTC |
| | | CAGAGATAATGCAAAAAGCACCCTATATCTACAAATGAACAGTCTGAGGTCTG |
| | | ACGACACGGCCACTTATTACTGTACAAGAGGTCGGATGCTACGGATCATTAGC |
| | | TTCTTTGATTACTGGGGCCAAGGAGTCATGGTCACAGTCTCTTCA |
| 314 | ZNRF3-253 (VL | GACATCCAGATGACACAGTCTCCAGCTTCCCTGTCAGCATCTCTTGGAGAAAC |
| | DNA) | TGTCTCCATCGAATGTCTAGCAAGTGAGGACATTGCCAATGATTTAGCGTGGT |
| | | ATCAACAGAAGTCAGGGAAATCTCCTCAGGTCCTGATCTATGCTGCAAGTAGG |
| | | GTGCAAGACGGGGTCCCATCACGGTTCAGTGGCAGTGGATCTGGCACACGGTA |
| | | TTCTCTCAAGATCACCGACATGCAACCTGAAGATGTGGCAGATTATTTCTGTC |
| | | AACAGAGTTACGGCTTTCCGCTCACGTTCGGTTCTGGGACCAAGCTAGAGATC |
| | | AAA |
| 315 | ZNRF3-253 (VH | EVQLVESGGGLVQPERSMKLSCAASGFTFSNYGMAWVRQAPTKGLEWVATINS |
| | prot) | DGSGTYYRGSVKGRFTISRDNAKSTLYLQMNSLRSDDTATYYCTRGRMLRIIS |
| | | FFDYWGQGVMVTVSS |
| 316 | ZNRF3-253 (VL | DIQMTQSPASLSASLGETVSIECLASEDIANDLAWYQQKSGKSPQVLIYAASR |
| | prot) | VQDGVPSRFSGSGSGTRYSLKITDMQPEDVADYFCQQSYGFPLTFGSGTKLEI |
| | | K |
| 317 | ZNRF3-254 (VH | GAGGTGCAACTGGTAGAGTCTGGGGGCGGTCTAGTGCAGCCTGAAAGGTCCAT |
| | DNA) | GAAACTCTCCTGTGCAGCCTCAGGATTCACTTTCAATAACTATGGCATGGCCT |
| | | GGGTCCGCCAGACTCCAACGAAGGGACTGGAATGGGTTGCAACCATTAATTCT |
| | | GATGGTAGTGGCACTCACTATCGAGGCTCCGTGAAGGGCCGATTCACTATCTC |
| | | CAGAGATAATGCAAAAGGCACCCTATATCTACAAATGCACAATCTGAGGTCTG |
| | | ACGACACGGCCACTTATTACTGTGCACGAGGTCGGATACTTCGGATCATTACC |
| | | TACTTTGATTCCTGGGGCCAAGGAGTCGTGGTCACAGTCTCCTCA |
| 318 | ZNRF3-254 (VL | GACATCCAGATGACACAGTCTCCAGCTTCCCTGTCAGCATCTCTTGGAGAAAG |
| | DNA) | TGTCTCCATCGAATGTCTAGCAAGTGAGGACATTGCCAATGATTTAGCGTGGT |
| | | ATCAACAGAAGTCAGGGAGATCTCCTCAGGTCCTGATCCATGCTGCAAGTAGG |
| | | GTGCAAGACGGGGTCCCATCACGGTTCAGTGGCAGTGGATCTGGCACACGGTA |
| | | CTCTCTCAAGATCACCGACATGCAACCTGAAGATGTAGCAGATTATTTCTGTC |
| | | AACAGAGTTACGGCTTTCCGCTCACGTTCGGTTCTGGGACCAAGCTAGAGATC |
| | | AAA |
| 319 | ZNRF3-254 (VH | EVQLVESGGGLVQPERSMKLSCAASGFTENNYGMAWVRQTPTKGLEWVATINS |
| | prot) | DGSGTHYRGSVKGRFTISRDNAKGTLYLQMHNLRSDDTATYYCARGRILRIIT |
| | | YFDSWGQGVVVTVSS |
| 320 | ZNRF3-254 (VL | DIQMTQSPASLSASLGESVSIECLASEDIANDLAWYQQKSGRSPQVLIHAASR |
| | prot) | VQDGVPSRFSGSGSGTRYSLKITDMQPEDVADYFCQQSYGFPLTFGSGTKLEI |
| | | K |
| 321 | ZNRF3-255 (VH | GAGGTGCAGCTGGTGGAGTCGGGGGGAGGCTTAGTGCAGCCTGGAAGGTCCCT |
| | DNA) | GAAACTCTCCTGCGCAGCCTCAGGATTCACTTTCAGTAGCTTTACAATGGCCT |
| | | GGGTCCGCCAGGCTCCAAAGAAGGGTCTGGAGTGGGTCGCATCCATTAGTTCT |
| | | GGTGGTGGTGGCACTTACTATCCAGATTCCGTGAAGGGCAGATTTACTATCTC |
| | | CAGAGATAATGCAAAAAGCACCCTATACCTGCAAATGGACAGTCTGAGATCTG |
| | | AGGACACGGCCAGTTACTATTGTGCAACCGGGGGGTTTGCTTACTGGGGCCAG |
| | | GGCACTCTGGTCATTGTCTCTTCA |
| 322 | ZNRF3-255 (VL | GACATCCAGATGACCCAGTCTCCATCTTCCCTGCCTGCATCTCTGGGAGACAG |
| | DNA) | AGTCACTATTACTTGCCGGGCAAGTCAAGACATTGGAAGTTATTTAAGATGGT |
| | | TCCAGCAGAAACCGGGGAAGTCTCCTAGGCTTATGATTTATGGTGCAACCAGC |
| | | TTGGCAAATGGGGTCCCATCAAGGTTCAGTGGCAGTAGGTCTGGGTCAGATTA |
| | | TTCTCTGACCATCAACAACTTGGAGTCTGAAGATATGGCTATTTATTACTGTC |
| | | TGCAGCATAATGAGTTTCCGTACACGTTTGGAACTGGGACCAAGCTGGAACTG |
| | | AAA |
| 323 | ZNRF3-255 (VH | EVQLVESGGGLVQPGRSLKLSCAASGFTFSSFTMAWVRQAPKKGLEWVASISS |
| | prot) | GGGGTYYPDSVKGRFTISRDNAKSTLYLQMDSLRSEDTASYYCATGGFAYWGQ |
| | | GTLVIVSS |
| 324 | ZNRF3-255 (VL | DIQMTQSPSSLPASLGDRVTITCRASQDIGSYLRWFQQKPGKSPRLMIYGATS |
| | prot) | LANGVPSRFSGSRSGSDYSLTINNLESEDMAIYYCLQHNEFPYTFGTGTKLEL |
| | | K |
| 325 | ZNRF3-265 (VH | GAGGTGCAGTTGGTAGAGTCTGGGGGCGGTTTAGTGCAGCCTGGAAGGTCCAT |
| | DNA) | GAAACTCTCCTGTGTAGCCTCAGGATTCAGTTTCAGTAAATATGGCATGGCCT |
| | | GGGTCCGCCAGGCTCCGACGAAGGCTTTGGAGTGGGTTGCAATCATTAGTCAG |
| | | GATGGTAGTAGAATTTACTATGGAGGCTCCGTGAAGGGCCGATTCACTCTCTC |
| | | CAGAGATAATGAAAAAAGCACCCTACACCTGCACATGAACAGTCTGAGGTCTG |
| | | AGGACACGGCCACTTATTATTGTGCAAGAGGAAGTATACTACGGATGATAACT |
| | | TTCTTTGATTATTGGGGCCAAGGAGTCACGGTCACAGTCTCCTCA |
| 326 | ZNRF3-265 (VL | GACATCCAGATGACTCAGTCTCCAGCTTCCCTGTCTGCATCTCTTGGAGAAAC |
| | DNA) | TGTCTCCATCGAATGTCTAGCAAGTGAGGGCATTTCCAATGATTTAGCGTGGT |
| | | ATCAGCAGAAGTCAGGGAAATCTCCTCGGCTCCTGATCTATGCTGCAAGTAGG |
| | | TTGCAAGACGGGGTCCCATCACGGTTCAGTGGCAGTGGATCTGACACACGGTT |
| | | TTCTCTCAAGATCAGCGGCATGCACCCTGAAGATGAAGCAGATTATTTCTGTC |
| | | AACAGAGTTACGGTTATCCGCTCACGTTCGGTTCTGGGACCAAGCTGGAGATC |
| | | AAA |
| 327 | ZNRF3-265 (VH | EVQLVESGGGLVQPGRSMKLSCVASGFSFSKYGMAWVRQAPTKALEWVAIISQ |
| | prot) | DGSRIYYGGSVKGRFTLSRDNEKSTLHLHMNSLRSEDTATYYCARGSILRMIT |
| | | FFDYWGQGVTVTVSS |
| 328 | ZNRF3-265 (VL | DIQMTQSPASLSASLGETVSIECLASEGISNDLAWYQQKSGKSPRLLIYAASR |
| | prot) | LQDGVPSRFSGSGSDTRFSLKISGMHPEDEADYFCQQSYGYPLTFGSGTKLEI |
| | | K |
| 329 | ZNRF3-269 (VH | GAGATACAACTTCAGGAGTCAGGACCTGGCCTTGTGAAACCTTCACAGTCACT |
| | DNA) | CTCCCTCACTTGTTCTGTCACTGATGACACCATTACCAGTGGTTATGATTGGA |
| | | GCTGGATCCGGAAGTTCCCGGGAAATAAGATGGAGTGGATGGGGGACATAAGC |
| | | AGCAGCGGGCACACGAACTACAACCCTTCGCTCCAGATTCGCATCTCCATAAC |
| | | TAGAGACACATCCAAGAATCAGTTCTTTCTGCAGTTGAACTCTGTAACAACTG |
| | | AGGATTCAGCCACCTATTATTGTGCTAGAGGCGGTTTTTTATTTCCTTTTACT |
| | | TTCTGGGGCCAAGGCACTCTGGTCACTGTCTCTTCA |
| 330 | ZNRF3-269 (VL | GACATCCAGATGACCCAGTCTCCATCCTCCCTGCCTGCATCTCTGGGAGAGAG |
| | DNA) | AGTCACCATCAGTTGCAGAGCAAGTCAGGTTCTTAACAATAATTTAAACTGGT |
| | | ATCAGCAGAAACCAGATGGAACGATTAAACCCCTACTCTACTACACATCCAAT |
| | | TTACAATCTGGTGTCCCATCAAGGTTCAGTGGCAGTGGGTCTGGGACAGATTA |
| | | TTCTCTCACCATCACCAGCCTTGACCCTGAAGATTTTGCAATATATTACTGCC |
| | | AACAGGATGCTAGTTTTCCTCCGACGTTCGGTGGCGGCACCAAGCTGGAATTG |
| | | AAA |
| 331 | ZNRF3-269 (VH | EIQLQESGPGLVKPSQSLSLTCSVTDDTITSGYDWSWIRKFPGNKMEWMGDIS |
| | prot) | SSGHTNYNPSLQIRISITRDTSKNQFFLQLNSVTTEDSATYYCARGGFLFPFT |
| | | FWGQGTLVTVSS |
| 332 | ZNRF3-269 (VL | DIQMTQSPSSLPASLGERVTISCRASQVLNNNLNWYQQKPDGTIKPLLYYTSN |
| | prot) | LQSGVPSRFSGSGSGTDYSLTITSLDPEDFAIYYCQQDASFPPTFGGGTKLEL |
| | | K |
| 333 | ZNRF3-270 (VH | GAGGTACAGCTGGTGGAGTCTGGAGGAGGCTTAGTGCAGCCTGGAGAGTCCCT |
| | DNA) | GAAACTCTCCTGTTCAGCCTCTGGATTCACATTCAGTAACTATGGCATGCACT |
| | | GGATACGCCAAGTTCCAGGAAAGGGGCTAGATTGGGTTGCATACATTACTAAT |
| | | AGCGGCGGTTCACTCTATGCAGCCGCTGTGAAGGGCCGGTTCACCATCTCCAG |
| | | AGACAATGCAAAGAACACCCTGTACCTGGAACTCACCAGTCTGAAGCCTGAGG |
| | | ACACTGCCATCTATCACTGTGCAAGAGGTTACAGTTATCCGGGACCTTACTGG |
| | | GGCCGAGGCACTCTGGTCACGGTCTCTTCA |
| 334 | ZNRF3-270 (VL | GATATTCAAGTGACTCAATCTCCATCCTCCCTCTTGGCATCTCTAGGAGAGAG |
| | DNA) | AGTCACTATCACATGCCAGACAGGTCAGAGCATTAACAATTACCTAAACTGGT |
| | | ATCAGCAGAGGCCAGGACAAGCTCCTCTACTCTTGATCTATTTTGCAACCAGT |
| | | TTGCAGACTGGCGTGCCATCAAGGTTCAGTGGCCAATATTCTGGGAGAAGTTT |
| | | CACTCTAACCATCACTAGCCTGGAGCCAGAAGATATTGCAAATTATTTTTGTC |
| | | TGCAGCATTACAGTCCTCCGTACACGTTTGGAGCTGGGACCAAGCTGGAACTG |
| | | AGA |
| 335 | ZNRF3-270 (VH | EVQLVESGGGLVQPGESLKLSCSASGFTFSNYGMHWIRQVPGKGLDWVAYITN |
| | prot) | SGGSLYAAAVKGRFTISRDNAKNTLYLELTSLKPEDTAIYHCARGYSYPGPYW |
| | | GRGTLVTVSS |
| 336 | ZNRF3-270 (VL | DIQVTQSPSSLLASLGERVTITCQTGQSINNYLNWYQQRPGQAPLLLIYFATS |
| | prot) | LQTGVPSRFSGQYSGRSFTLTITSLEPEDIANYFCLQHYSPPYTFGAGTKLEL |
| | | R |
| 337 | ZNRF3-275 (VH | CAGGTGCAGCTGAAGGAGTCAGGACCTGGCCTTGTGCAGCCCTCACAGACCCT |
| | DNA) | GTCCCTCACCTGCACTGTCTCTGGGTTCTCATTTAACTACTATAATGTTCACT |
| | | GGCTTCGACAGCCTCCAGGAAAGGGTCTGGAGTGGTTGGGAGTAATCTGGAGT |
| | | GGTGGAAACAGAGATTACAATTCAGCTCTCAAACCCCGACTGCGCATCAGCAG |
| | | GGACACCTCCAAGACCCAAGTTTTCTTAACATTGAACAGTCTGCAGACTGAGG |
| | | ACACAGGCATTTACTATTGTACCAGAATGTGGGGCTATGGGTACAACCCCTTT |
| | | GATCACTGGGGCCAAGGAGTCATGGTCACAGTCTCCTCA |
| 338 | ZNRF3-275 (VL | GACATCCAGATGACTCAGTCTCCGTCATTTCTGTCTGCATCTCTTGGAGACAA |
| | DNA) | CATCACCATCACTTGCCATGCCAGTCAGGACATCAAGGGTTGGTTAGCCTGGT |
| | | ACCAACAAAAGTCAGGGAATGCTCCTGAACTGTTGATTTATAAGGCATCTAGC |
| | | CTGCAATCAGGGGTTCCATCAAGATTCAGTGGCGGCGGATCTGGAACAGAATA |
| | | TGTTCTCACTATCAGCAACCTACAGCCTGACGATATTGCCACTTATTACTGTC |
| | | AGCTTTATCACAGCTTTCCATTCACGTTTGGCTCAGGGACGAAGTTGGAAACA |
| | | AAA |
| 339 | ZNRF3-275 (VH | QVQLKESGPGLVQPSQTLSLTCTVSGFSFNYYNVHWLRQPPGKGLEWLGVIWS |
| | prot) | GGNRDYNSALKPRLRISRDTSKTQVFLTLNSLQTEDTGIYYCTRMWGYGYNPF |
| | | DHWGQGVMVTVSS |
| 340 | ZNRF3-275 (VL | DIQMTQSPSFLSASLGDNITITCHASQDIKGWLAWYQQKSGNAPELLIYKASS |
| | prot) | LQSGVPSRFSGGGSGTEYVLTISNLQPDDIATYYCQLYHSFPFTFGSGTKLET |
| | | K |
| 341 | ZNRF3-279 (VH | CAGGTCCAGCTGCAGCAGTCTGGAGCTGAGCTGGTGAAGCCTGGGACTTCTGT |
| | DNA) | GAGGCTGTCCTGCAAGGCTTCTGGCTACACCTTTACTCACAACCATATGAACT |
| | | GGATAAAGCAGACGACTGGACAGGGCCTTGAGTGGATTGGAATTATTAATCCG |
| | | GGAAGTGGAGGTACTAATTACAATGTGAGGTTCAAGGGCAAGGCCACATTGAC |
| | | TGTAGACACATCCTCCAGCACAGCCTTCATGCAACTCAGCAGCCTGACACCTG |
| | | AGGACTCTGCGGTCTATTACTGTGCAAGAAACCTTATTACTATAGCAGGCCCG |
| | | TTTGCTTACTGGGGCCAAGGCACTCTGGTCACTGTCTCTTCA |
| 342 | ZNRF3-279 (VL | GACACTGTACTGACCCAGTCTCCTGCTTTGACTGTGTCTCCAGGAGAGAGGGT |
| | DNA) | TTCCATCTCCTGTAGGGCCAGTGAAGGGGTCAGTTCATATATACACTGGTACC |
| | | AACAGAAACCAGGACAGCAGCCCAAACTCCTCATCTATAAAGCATCAAACCTA |
| | | GCATCTGGGGTCCCTGCCAGGTTCAGTGGCAGTGGGTCTGGGACAGACTTCAC |
| | | CCTCACCATTGATCCTGTGGAGGCTGATGACACTGCAACCTATTTCTGTCAGC |
| | | AGAGTTGGATTGATCACACGTTTGGAACTGGGACCAAGCTGGAACTGAAA |
| 343 | ZNRF3-279 (VH | QVQLQQSGAELVKPGTSVRLSCKASGYTFTHNHMNWIKQTTGQGLEWIGIINP |
| | prot) | GSGGTNYNVRFKGKATLTVDTSSSTAFMQLSSLTPEDSAVYYCARNLITIAGP |
| | | FAYWGQGTLVTVSS |
| 344 | ZNRF3-279 (VL | DTVLTQSPALTVSPGERVSISCRASEGVSSYIHWYQQKPGQQPKLLIYKASNL |
| | prot) | ASGVPARFSGSGSGTDFTLTIDPVEADDTATYFCQQSWIDHTFGTGTKLELK |
| 345 | ZNRF3-287 (VH | GAGGTGCAGTTGGTGGGGTCTGGTGGAGGCTTGGTGCAGCCTGGAAGGTCTCT |
| | DNA) | GAAAATCTCCTGTGCAGCCTCAGGATTCACTTTCAGTTACTATGGCATGGCCT |
| | | GGGTCCGCCAGGCTCCAACGAGGGGGCTGGAGTGGGTCGCAACCATTACTTAT |
| | | AATGGTTCTAAGTCTTACTATCGAGACTCCGTGAAGGGCCGATTCACTATCTC |
| | | CAGAGAGTATGCAAAAAGCACCCTATACCTCCAAATGGACGATCTGACGTCTG |
| | | AAGACACGGCCACTTATTTCTGTGCAAGACGGACTACGGGTATGCTCTTTGAT |
| | | TACTGGGGCCAAGGAGTCTTGGTCACAGTCTCCTCA |
| 346 | ZNRF3-287 (VL | CAGGCTGTCCTTACTCAGCCAAACTCTGTGTCTATGTCTCTAGGAAGCACAGT |
| | DNA) | CAAGCTGTCTTGCACACTCAACTCTGGTAACATAGAAAACAACTTTGTGCACT |
| | | GGTACCAACAATATGAGGGAAGATCTCCCACCACTATGATTTATAATGATGAT |
| | | AAGAGACCGGATGGTGTCCCTGACAGGTTCTCTGGCTCCATTGACAGCTCTTC |
| | | CAACTCAGCCTTCCTGACAATCAATAATGTGGAAATTGGAGATGAAGCTATCT |
| | | ACTTCTGTCATTCTTTTGTTAGTAGTATTCATATTTTCGGCGGTGGAACCAAG |
| | | CTCACTGTCCTA |
| 347 | ZNRF3-287 (VH | EVQLVGSGGGLVQPGRSLKISCAASGFTFSYYGMAWVRQAPTRGLEWVATITY |
| | prot) | NGSKSYYRDSVKGRFTISREYAKSTLYLQMDDLTSEDTATYFCARRTTGMLFD |
| | | YWGQGVLVTVSS |
| 348 | ZNRF3-287 (VL | QAVLTQPNSVSMSLGSTVKLSCTLNSGNIENNFVHWYQQYEGRSPTTMIYNDD |
| | prot) | KRPDGVPDRFSGSIDSSSNSAFLTINNVEIGDEAIYFCHSFVSSIHIFGGGTK |
| | | LTVL |
| 349 | ZNRF3-296 (VH | GAGGTACAGCTGGTGGAGTCTGGAGGAGGCTTAGTGCAGCCTGGAAAGTCCCT |
| | DNA) | GAGACTCTCCTGTTCAGCCTCTGGATTCACATTCAGTAACTATGGCCTGCACT |
| | | GGATACGCCAAGCTCCAGGAAAGGGACTAGATTGGCTTGGATATAGTAGTAGT |
| | | AACAGCGGTACAGTCTATGGAGACGCTGTGAAGGGCCGGGTCACCATCTCCAG |
| | | AGACAATGCAAAGAACACCCTGTACCTGCAACTCAACAGTCTGAAGTCTGAAG |
| | | ACACTGCCATCTATTATTGTGCAAGAGGCTATAGCAGCTCCCCGTTTGCTTAC |
| | | TGGGGCCAAGGCACTCTGGTCACTGTCTCTTCA |
| 350 | ZNRF3-296 (VL | GACATTGTGCTGACCCAGTCTCCTGCTTTGGCTGTGTCTCTAGGCCAGAGAGT |
| | DNA) | CACCATCTCATGCAAAGCCAGCCAGAATGTCGATGGTTTTGGCATTGGTTATA |
| | | TGCACTGGTACCAACAGAAACCAGGACAGAAACCCAAACTCCTCATCTTTGCA |
| | | GGATCCAACTTAGCATCTGGGATCCCTGCCAGGTTCAGTGGCAGTGGGTCTGG |
| | | GACAGACTTCACCCTCACCATCGATCCTGTGGAGACTGATGATATTGCAACCT |
| | | ATTACTGTCAGCAGAATAAAGATTTTCCCCTCACGTTCGGTTCTGGGTCCAGG |
| | | CTGGAGATCACA |
| 351 | ZNRF3-296 (VH | EVQLVESGGGLVQPGKSLRLSCSASGFTFSNYGLHWIRQAPGKGLDWLGYSSS |
| | prot) | NSGTVYGDAVKGRVTISRDNAKNTLYLQLNSLKSEDTAIYYCARGYSSSPFAY |
| | | WGQGTLVTVSS |
| 352 | ZNRF3-296 (VL | DIVLTQSPALAVSLGQRVTISCKASQNVDGFGIGYMHWYQQKPGQKPKLLIFA |
| | prot) | GSNLASGIPARFSGSGSGTDFTLTIDPVETDDIATYYCQQNKDFPLTFGSGSR |
| | | LEIT |
| 353 | ZNRF3-30 (VH | GAGGTGCAGCTGGCGGAGTCTGGGGGAGGCTTAGAGCAGCCTGGAAGGTCCCT |
| | DNA) | GAAACTCTCCTGTGCAGCCTCAGGATTCACTTTCAGTAACTATGACATGGCCT |
| | | GGGTCCGCCAGGCTCCAACGAGGGGTCTGGAGTGGGTCGCATCCATTCTTACT |
| | | AGTGGTGGTACTACTTATTATCGAGACTCCGTGAAGGGCCGATTCACTGTCTC |
| | | CAGAGATAATGCAAAAAGCACCCTATACCTGCAAATGGACAGTCTGAGGTCTG |
| | | AGGACACGGCCACTTATTACTGTGCAAGACATGGGGTCGGTAGCTACGATTGG |
| | | TTTGCTGACTGGGGCCAAGGCACTCTGGTCACTGTCTCTTCA |
| 354 | ZNRF3-30 (VL | GACATTGTCTTGACCCAGTCTCCTGCTTTGGCTGTGTCTCTAGGGCAGAGGGC |
| | DNA) | CACAATCTCCTGTAGGGCCAGCCAAAGTGTCACTATATCTGGATATAATCTTA |
| | | TGCACTGGTACCAACAGAAACCAGGACAACAACCCAAACTCCTCATCTATCGT |
| | | GCATCCAACCTAGCATTTGGGATCCCTGCCAGGTTCAGTGGCAGTGGGTCTGG |
| | | GACAGACTTCACCCTCACCATCAATCCTGTGCAGGCTGATGATATTACGACCT |
| | | ATTACTGTCAGCAGAGTAGGAAGTCTCGGACGTTCGGTGGAGGCACCAGACTG |
| | | GAATTGAAA |
| 355 | ZNRF3-30 (VH | EVQLAESGGGLEQPGRSLKLSCAASGFTFSNYDMAWVRQAPTRGLEWVASILT |
| | prot) | SGGTTYYRDSVKGRFTVSRDNAKSTLYLQMDSLRSEDTATYYCARHGVGSYDW |
| | | FADWGQGTLVTVSS |
| 356 | ZNRF3-30 (VL | DIVLTQSPALAVSLGQRATISCRASQSVTISGYNLMHWYQQKPGQQPKLLIYR |
| | prot) | ASNLAFGIPARFSGSGSGTDFTLTINPVQADDITTYYCQQSRKSRTFGGGTRL |
| | | ELK |
| 357 | ZNRF3-300 (VH | GAGGTGCAGCTGGTAGAGTCTGGGGGCGGTTTAGTACAGCCTGAAAGGTCCAT |
| | DNA) | GAAACTCTCCTGTGCAGCCTCAGGATTCACTTTCAGTAACTATGGCATGGCCT |
| | | GGGTCCGCCAGGCTCCAACGAAGGGGCTGGAGTGGGTTGCAACCATTAATTCT |
| | | GATGGTAGTGGCACTTACTATCGAGGCTCCGTGAAGGGCCGATTCACTATCTC |
| | | CAGAGATAATGCAAGAAACACCCTATACCTAGAAATGAACAGTCTGCGGTCTG |
| | | ACGACACGGCCACTTATTACTGTACAAGAGGTCGGATACTACGGATTATTACC |
| | | TACTTTGATTACTGGGGCCAAGGAGTCATGGTCACAGTCTCCTCA |
| 358 | ZNRF3-300 (VL | GACATCCAGATGACACAGTCTCCAGCTTCCCTGTCAGCATCTGTTGGAGAAAC |
| | DNA) | CGTCTCCATCGAATGTCTAGCAAGTGAGGACATTGCCAATGATTTAGCGTGGT |
| | | ATCAGCAGAAGTCAGGGAAATCTCCTCAGGTCCTGATCTATGCTGCAAGTAGG |
| | | GTGCAAGACGGGGTCCCATCACGGTTCAGTGGCAGTGGATCTGGCACACGATA |
| | | TTCTCTCAGGATCAGCGACATGCAACCTGAAGATGTAGCAGATTATTTCTGTC |
| | | AACAGAGTTACAGCTTTCCGCTCACGTTCGGATCTGGGACCAAGCTGGAGATC |
| | | AAA |
| 359 | ZNRF3-300 (VH | EVQLVESGGGLVQPERSMKLSCAASGFTFSNYGMAWVRQAPTKGLEWVATINS |
| | prot) | DGSGTYYRGSVKGRFTISRDNARNTLYLEMNSLRSDDTATYYCTRGRILRIIT |
| | | YFDYWGQGVMVTVSS |
| 360 | ZNRF3-300 (VL | DIQMTQSPASLSASVGETVSIECLASEDIANDLAWYQQKSGKSPQVLIYAASR |
| | prot) | VQDGVPSRFSGSGSGTRYSLRISDMQPEDVADYFCQQSYSFPLTFGSGTKLEI |
| | | K |
| 361 | ZNRF3-301 (VH | CAGGTACAGCTGAAGGAGTCAGGACCTGGGCTGGTGCAGCCCTCACAGACCCT |
| | DNA) | GTCCCTCACCTGCACTGTCTCTGGGTTCTCACTAACCAGCTACAGTGTACACT |
| | | GGGTTCGCCAGCACTCAGGAAAGAGTCTGGAATGGATGGGAAGAATATGGAGT |
| | | GATGGAGACACATCATATAATTCAGCGTTCACATCCCGACTGAGCATCAGCAG |
| | | GGACACCTCCAAGAGCCAAGTTTTCTTAAAAATGAACAGTCTCCAAACTGAGG |
| | | ACACAGGCACTTACTACTGTGACAGATCGGGTGGGAGCCCTTTCTTTGATTAT |
| | | TGGGGCCAAGGAGTCATGGTCACAGTCTCCTCA |
| 362 | ZNRF3-301 (VL | CAGTTTGTGCTTACTCAGTCAAACTCTGTGTCTACGAATCTCGGAAGCACAGT |
| | DNA) | CAAACTGTCTTGCGAGCGCAGCATTGGTAACATTGGAAGCAATTATGTGAATT |
| | | GGTACCAGCAGTATGAGGGAAGATCTCCCACCACTATGATTTATAAGTTTGAT |
| | | CAGAGACCTGATGGAGTTCCTGACAGGTTCTCTGGCTCCATTGACAGATCTTC |
| | | CAATTCAGTCCTCCTGACAATCAATAATGTGCAGACTGAAGATGAAGCTGACT |
| | | ACTTCTGTCAGTCTTACAGTAGTGATATGTATATTTTCGGCGGTGGAACCAAG |
| | | CTCACTGTCCTA |
| 363 | ZNRF3-301 (VH | QVQLKESGPGLVQPSQTLSLTCTVSGFSLTSYSVHWVRQHSGKSLEWMGRIWS |
| | prot) | DGDTSYNSAFTSRLSISRDTSKSQVFLKMNSLQTEDTGTYYCDRSGGSPFFDY |
| | | WGQGVMVTVSS |
| 364 | ZNRF3-301 (VL | QFVLTQSNSVSTNLGSTVKLSCERSIGNIGSNYVNWYQQYEGRSPTTMIYKED |
| | prot) | QRPDGVPDRFSGSIDRSSNSVLLTINNVQTEDEADYFCQSYSSDMYIFGGGTK |
| | | LTVL |
| 365 | ZNRF3-305 (VH | GAGGTCCTGCTGCAGCAGTCAGGCGCTGAGCTTGTGAGACCTGGGTCCTCAGT |
| | DNA) | CAAGATTTCTTGCAAGACTTCTGGCTACACCTTTACAGATTACTATTTGCACT |
| | | GGGTGAAGCAGAGGCCTGAACAGGGCCTGGTATGGATAGGACGGATTAATCCT |
| | | ATAAATGGAAAAACTGTTTGTATAGAGAAGTTCAAAACCAAGGCCACACTGAC |
| | | TGCAGATAAATCGTCCAACACAGCCTACATGGAACTCAGCAGCCTGACATCTG |
| | | AAGACACCGCAACCTATTTTTGTACTAGAGGAGGGCTTGCTTACTGGGGCCAA |
| | | GGCACTCTGGTCACTGTCTCTTCA |
| 366 | ZNRF3-305 (VL | GACATCCAGATGACCCAGTCTCCATCGTCCCTGCCTGCATCTCTGGGAGAGAG |
| | DNA) | AGTCACCATCAGTTGTAGAGCAAGTCAGGGTATTAGCAATAATTTAAACTGGT |
| | | ATCAGCAGAAACCAGATGGAACGATTAAACCCCTGATCTACTACACATCCAAT |
| | | TTACAATCTGGTGTCCCATCAAGGTTCAGTGGCAGTGGGTCTGGGACAGATTA |
| | | TTCTCTCACCATCGGCAGCCTTGAGCCTGAAGATTTTACAATGTATTACTGCC |
| | | AACAGGATGCTAGTTTTCCTCCCACGTTTGGAGCTGGGACCAAGCTGGAACTG |
| | | AAA |
| 367 | ZNRF3-305 (VH | EVLLQQSGAELVRPGSSVKISCKTSGYTFTDYYLHWVKQRPEQGLVWIGRINP |
| | prot) | INGKTVCIEKFKTKATLTADKSSNTAYMELSSLTSEDTATYFCTRGGLAYWGQ |
| | | GTLVTVSS |
| 368 | ZNRF3-305 (VL | DIQMTQSPSSLPASLGERVTISCRASQGISNNLNWYQQKPDGTIKPLIYYTSN |
| | prot) | LQSGVPSRFSGSGSGTDYSLTIGSLEPEDFTMYYCQQDASFPPTFGAGTKLEL |
| | | K |
| 369 | ZNRF3-312 (VH | CAGGTACAACTGCAGCAGTCTGGAGCTGAGTTGGTGAAGCCTGGGTCTTCAGT |
| | DNA) | GAAGATGTCCTGCAAGGCTTCTGGCTACAGTTTCACCACCTACTATATCTATT |
| | | GGATAAATCAGAGGCCTGGACAGGGCCTTGAGTGGATTGGGCGTATTGGTCTT |
| | | GGAAGTGGAGATACTGATTACAATGAGAAATTCAAGGGCAAGGCCACATTTAC |
| | | TGTAGACAAATTTTCCAACACAGCCTACCTGCAACTCAGCAGCCTGACACCTG |
| | | AGGACACTGCGGTCTATTACTGTGCAAGATGGGACTACGGGTATAGGGGGTTT |
| | | GCTTACTGGGGCCAGGGCACTCTGGTCACTGTCTCTTCA |
| 370 | ZNRF3-312 (VL | GATGTTGTGATGACACAAACTCCAGTCTCCCTGTCTGTCAGCCTTGGAGGTCA |
| | DNA) | AGTCTCTATCTCTTGCCGGTCTAGTCAGAGCTTTGTACACAGTGATGGAAATA |
| | | CCTATTTGAATTGGTACCTGCAGACGCCCGGCCAATCTCCACAACTCCTCATC |
| | | TATAAGGTTTCCAACCGATTATCTGGGGTCCCAGATAGGTTCAGTGGTAGTGG |
| | | TTCAGGGACATATTTCACCCTCAAGATCAGCAGAGTCGAGCATGATGATTTGG |
| | | GAGTTTATTACTGCGGCCAGGCTTCAAAAATTCCGCTCACGTTCGGTTCCGGG |
| | | ACCAAGCTGGAGATCAAA |
| 371 | ZNRF3-312 (VH | QVQLQQSGAELVKPGSSVKMSCKASGYSFTTYYIYWINQRPGQGLEWIGRIGL |
| | prot) | GSGDTDYNEKFKGKATFTVDKFSNTAYLQLSSLTPEDTAVYYCARWDYGYRGF |
| | | AYWGQGTLVTVSS |
| 372 | ZNRF3-312 (VL | DVVMTQTPVSLSVSLGGQVSISCRSSQSFVHSDGNTYLNWYLQTPGQSPQLLI |
| | prot) | YKVSNRLSGVPDRFSGSGSGTYFTLKISRVEHDDLGVYYCGQASKIPLTFGSG |
| | | TKLEIK |
| 373 | ZNRF3-314 (VH | CAGGTGCAGCTGAAGGAGTCAGGACCTGGCCTTGTGCAGCCCTCACAGACCCT |
| | DNA) | GTCCCTCACCTGCAATGTCTCTGGTTTCTCATTTAACTACTATAATGTACACT |
| | | GGGTTCGACAGCCTCCAGGAAAGGGTCTGGAGTGGTTGGGAGTAATCTGGAGT |
| | | GGTGGAAACACAGATTACAATTCAGCTCTCAAACCCCGACTGCGCATCAGCAG |
| | | GGACACCTCCAAGAGCCAAGTTTTCTTAACATTGAACAGTCTGCAGACTGAGG |
| | | ACACAGGCATTTACTATTGTAACAGAATGTGGGGCTATGGGTATAACCCCTTT |
| | | GATTACTGGGGCCAAGGAGTCATGGTCACAGTCTCCTCA |
| 374 | ZNRF3-314 (VL | GACATCCAGATGACTCAGTCTCCGTCATTTCTGTCTGCATCTCTTGGAAACAA |
| | DNA) | CATCACCATCACTTGCCATGCCAGTCAGGACATCAAGGGTTGGTTAGCCTGGT |
| | | ACCAACAAAAGTCAGGGAATGCTCCTGAACTGTTGATTTATAAGACATCTAGC |
| | | CTGCAATCAGGGGTTCCATCAAGATTCAGTGGCAGCGGATCTGGAACAGAATA |
| | | TGTTCTCACTATCAGCAACCTACAGCCTGACGATATTGCCACTTATTACTGTC |
| | | AGCTTTATCACAGCTTTCCATTCACGTTCGGCTCAGGGACGAAGTTGGAAATA |
| | | AAA |
| 375 | ZNRF3-314 (VH | QVQLKESGPGLVQPSQTLSLTCNVSGFSFNYYNVHWVRQPPGKGLEWLGVIWS |
| | prot) | GGNTDYNSALKPRLRISRDTSKSQVFLTLNSLQTEDTGIYYCNRMWGYGYNPF |
| | | DYWGQGVMVTVSS |
| 376 | ZNRF3-314 (VL | DIQMTQSPSFLSASLGNNITITCHASQDIKGWLAWYQQKSGNAPELLIYKTSS |
| | prot) | LQSGVPSRFSGSGSGTEYVLTISNLQPDDIATYYCQLYHSFPFTFGSGTKLEI |
| | | K |
| 377 | ZNRF3-322 (VH | GAGGTGCACCTGGTGGAGTCTGGGGGAGGCCTAGTGCAGCCTGGAAGGTCTCT |
| | DNA) | GACACTATCCTGTGTTGCCTCTGGATTCACATTCAATAACTGGTGGATGACCT |
| | | GGATCCGCCAGGCTCCAGGGAAGGGGCTGGAGTGGGTTGCATCCATTTCTCAC |
| | | ACTGGTGGTAGCACCTCCTATCCAGACTCTGTGAAGGGCCGTTTCACTATCTC |
| | | CAGAGATAATGCAAAAAGTACCCTATACCTGCAAATGACCAGTCTGAGGTCTG |
| | | ACGATACGGCCACTTATTACTGTACAAGATGGGGACCCATACTACGGGGCTTT |
| | | GATTACTGGGGCCAAGGAGTCATGGTCACAGTCTCCTCA |
| 378 | ZNRF3-322 (VL | GATGTTGTGCTGACCCAGACTCCACCCACTTTATCGGCTACCATTGGACAATC |
| | DNA) | CGTCTCCATCTCTTGCAGGTCAAGTCAGAGTCTCTTACATATTAATGGAAACA |
| | | CCTATGTAAATTGGTTGGTACAGAGGCCAGGCCAACCTCCACAACTTCTATTT |
| | | TATTTGGTATCCAGACTGGAATCTGGGGTCCCCAACAGGTTCAGTGGCAGTGG |
| | | GTCAGGAACTGATTTCACACTCAAAATCAGTGGAGTAGAGGCTGAGGATTTGG |
| | | GAGTTTATTACTGCGTGCAAAGTACCCATGCTCCTCCGACGTTCGGTGGAGGC |
| | | ACCAAGCTGGAATTGAAA |
| 379 | ZNRF3-322 (VH | EVHLVESGGGLVQPGRSLTLSCVASGFTFNNWWMTWIRQAPGKGLEWVASISH |
| | prot) | TGGSTSYPDSVKGRFTISRDNAKSTLYLQMTSLRSDDTATYYCTRWGPILRGF |
| | | DYWGQGVMVTVSS |
| 380 | ZNRF3-322 (VL | DVVLTQTPPTLSATIGQSVSISCRSSQSLLHINGNTYVNWLVQRPGQPPQLLE |
| | prot) | YLVSRLESGVPNRFSGSGSGTDFTLKISGVEAEDLGVYYCVQSTHAPPTFGGG |
| | | TKLELK |
| 381 | ZNRF3-329 (VH | GAGGTACAGCTGGTGGAGTCTGGAGGAGGCTTAGTGCAGCCTGGAAAGTCCCT |
| | DNA) | GAAACTCTCCTGTTCAGCCTCTGGATTCACATTCAGTAGCCATGGCATGCACT |
| | | GGATACGCCAAGCTCCAGGAAAGGGGCTAGATTGGGTTGCATACATTAGTAAT |
| | | AACAGCGGTACAATCTATTCAGCCGCTGTGAGGGGCCGGTTCACCATCTCCAG |
| | | AGACAATTCAAAGAACACCCTGTTCCTACAACTCAACAGTCTGAAGTCTGAAG |
| | | ACACTGCCATCTATTACTGTGCAAGAGGTTATAACTACCCGGGAGCTTACTGG |
| | | GGCCAAGGCACTCTGGTCACTGTCTCTTCA |
| 382 | ZNRF3-329 (VL | GATATTCAAGTGACTCAATCTCCATCCTCCCTCTTGGCATCTCTAGGAGAGAG |
| | DNA) | AGTCACTATCACATGCCAGACAAGTCAGAGCATTAGAAATTACCTAAACTGGT |
| | | ATCAGCAGAAACCAGGACAAGTTCCCATACTCTTGATCTATTATGCAACCAGT |
| | | TTGCAGACTGACATGCCATCAAGGTTCAGTGGCCAATATTCTGGGAGAAGTTT |
| | | CACTCTCACCATCACTAGCCTGGAGCCAGAAGACATTGCAAATTATTTTTGTC |
| | | TGCAGCATTACAGTGTTCCGTACACGTTTGGAGCTGGGACCAAGCTGGAACTG |
| | | AAA |
| 383 | ZNRF3-329 (VH | EVQLVESGGGLVQPGKSLKLSCSASGFTFSSHGMHWIRQAPGKGLDWVAYISN |
| | prot) | NSGTIYSAAVRGRFTISRDNSKNTLFLQLNSLKSEDTAIYYCARGYNYPGAYW |
| | | GQGTLVTVSS |
| 384 | ZNRF3-329 (VL | DIQVTQSPSSLLASLGERVTITCQTSQSIRNYLNWYQQKPGQVPILLIYYATS |
| | prot) | LQTDMPSRFSGQYSGRSFTLTITSLEPEDIANYFCLQHYSVPYTFGAGTKLEL |
| | | K |
| 385 | ZNRF3-35 (VH | GAGGTGCAGTTGGTGGAGTCTGGGGGAGGCTTAGTGCAGCCTGGAAGGTCCAT |
| | DNA) | GAAACTCTCCTGTGCAGCCTCAGGATTCACTTTCAGTAACTATTACATGGCCT |
| | | GGGTCCGCCAGGCTCCAAGGAAGGGTCTGGAGTGGGTCGCATCCATTAGTACT |
| | | GGTGGTGGTAACACTTACTATCGAGAATACGTGAGGGGCCGATTCACTATCTC |
| | | CAGAGATAATGCAAAAAGCACCCTATATCTGCAAATGGACAGTCTGAGGTCTG |
| | | AGGAAACGGCCACTTATTACTGTGCAAGACATTCGGGGGGTATAATCCGGGCC |
| | | CGGGGTCACTACTTTGATTACTGGGGCCAAGGAGTCATGGTCACAGTCTCCTC |
| | | A |
| 386 | ZNRF3-35 (VL | GACATCCAGATGACACAGTCTCCAGCTTCCCTGTCTGCATCTCTTGGAGAAAT |
| | DNA) | TGTCTCCATCGAATGTCTAGCAAGTGAGGGCATTTCCAATGATTTAGCGTGGT |
| | | ATCAGCAGAAGTCAGGGAAATCTCCTCAGGTCCTGATCTCTGCTGCAAGTAGG |
| | | TTGCAAGACGGGGTCCCATCACGGTTCAGTGGCAGTGGATCTGGCACACGGTA |
| | | TTCTCTCAAGATCAGCGGCATGCAACCTGAAGACGAAGCAGATTATTTCTGTC |
| | | AACAGAGTTACACGTATCCGTACACGTTTGGAGCTGGGACCAAGCTGGAACTG |
| | | AAA |
| 387 | ZNRF3-35 (VH | EVQLVESGGGLVQPGRSMKLSCAASGFTFSNYYMAWVRQAPRKGLEWVASIST |
| | prot) | GGGNTYYREYVRGRFTISRDNAKSTLYLQMDSLRSEETATYYCARHSGGIIRA |
| | | RGHYFDYWGQGVMVTVSS |
| 388 | ZNRF3-35 (VL | DIQMTQSPASLSASLGEIVSIECLASEGISNDLAWYQQKSGKSPQVLISAASR |
| | prot) | LQDGVPSRFSGSGSGTRYSLKISGMQPEDEADYFCQQSYTYPYTFGAGTKLEL |
| | | K |
| 389 | ZNRF3-55 (VH | GAGGTGCACCTGGTGGAGTCTGGGGGAGGCTTAGTGCAGCCTGGAGGGTCCCT |
| | DNA) | GAAACTCTCCTGTGCAGCCTCAGGATTCACTTTCAGTAACTATGACATGGCCT |
| | | GGGTCCGCCAGGCTCCAACGAGGGGTCTGGAGTGGGTCGCTTCCATTAGTCCT |
| | | GGTGGTGGAAAAACTTACTATCGAGACTCCGTGAAGGGCCGACTCACTATCTC |
| | | CAGAAACAATGCAGAAAATACCCAATACCTGCAAATTGACAGTCTGAGGTCTG |
| | | AAGACACGGCCACTTATTACTGTTCAAGACTCGGTCCGGCATACTCCGGAGAA |
| | | TGGTTTGCTTACTGGGGCCAAGGCACTCTGGTCACTGTCTCTTCA |
| 390 | ZNRF3-55 (VL | GACACTGTGCTGACCCAGTCTCCTGCTTTGACTGTGTCTCCAGGAGACAAAAT |
| | DNA) | TACCATCTCCTGTAGGGCCAGTGAAGGTGTCAATACACGTATACACTGGTACC |
| | | AACAGAAATCAGGACAGCAACCCAAACTCCTCATCTACGGTGCATCCAACTTA |
| | | GATTCTGGAGTCCCTGACAGGTTCAGTGGCAGTGGGTTTGGGACAGACTTTAC |
| | | CCTCACCATCGATCCTGTTGAGGCTAGTGATACTGCCACCTATTTCTGTCAGC |
| | | AGAGTTGGAATGTTCCGCACACGTTTGGAGGTGGGACCAAACTGGAACTGAAA |
| 391 | ZNRF3-55 (VH | EVHLVESGGGLVQPGGSLKLSCAASGFTFSNYDMAWVRQAPTRGLEWVASISP |
| | prot) | GGGKTYYRDSVKGRLTISRNNAENTQYLQIDSLRSEDTATYYCSRLGPAYSGE |
| | | WFAYWGQGTLVTVSS |
| 392 | ZNRF3-55 (VL | DTVLTQSPALTVSPGDKITISCRASEGVNTRIHWYQQKSGQQPKLLIYGASNL |
| | prot) | DSGVPDRFSGSGFGTDFTLTIDPVEASDTATYFCQQSWNVPHTFGGGTKLELK |
| 393 | ZNRF3-6 (VH | GAGGTACAGCTGGTGGAGTCTGGAGGAGGCTTAGTGCCGCCTGGAAAGTCCCT |
| | DNA) | GAAACTCTCCTGTTCAGCCTCTGGATTCCCATTCAGTAACTATGGCATGCACT |
| | | GGATTCGCCAAGCTCCAGGAAAGGGGCTTGATTGGGTTGGATACATTAGTAGT |
| | | AATAGTGGTACAATCTATGCAGACGCTGTGAAGGGCCGGTTCACCATCTCCAG |
| | | AGACAATGCAAAGAACACCCTGTACCTGCTAATCAACAGTCTGAAGTCTGAAG |
| | | ACACTGCCATGTATTACTGTGCAAGAGGTTATTTTGATGGTTATTATCGTTTC |
| | | TGGGGCCAAGGAGTCATGGTCACAGTCTCCTCA |
| 394 | ZNRF3-6 (VL | GACATTGTGATGACCCAGTCTCCATTCTCCCTGGCTGTGTCAGAAGGAGACAT |
| | DNA) | GGTCACTATAATGTGCAGGTCCAGTCAGAGTCTTCTATCCAGTGGAAACCAAA |
| | | AGAACTACTTGGCCTGGTACCAGCAGAAACCAGGGCAGTCTCCTAAACTACTG |
| | | ATCTACCATGCATCCACTAGACAATCAGGGGTCCCTGATCGCTTCATAGGCAG |
| | | TGGATCTGGGACAGACTTCACTCTGACCATCAGCGATGTGCAGGCTGAAGACC |
| | | TGGCAGATTATTACTGCCTGCAGCATTACAGTTCTCCCACGTTCGGCTCAGGG |
| | | ACGAAGTTGGAAATAAAA |
| 395 | ZNRF3-6 (VH | EVQLVESGGGLVPPGKSLKLSCSASGFPFSNYGMHWIRQAPGKGLDWVGYISS |
| | prot) | NSGTIYADAVKGRFTISRDNAKNTLYLLINSLKSEDTAMYYCARGYFDGYYRF |
| | | WGQGVMVTVSS |
| 396 | ZNRF3-6 (VL | DIVMTQSPFSLAVSEGDMVTIMCRSSQSLLSSGNQKNYLAWYQQKPGQSPKLL |
| | prot) | IYHASTRQSGVPDRFIGSGSGTDFTLTISDVQAEDLADYYCLQHYSSPTFGSG |
| | | TKLEIK |
| 397 | ZNRF3-90 (VH | GAGGTGCAGTTGGTGGAGTCTGGGGGAGGCTTAGTGCAGCCTGGAAGGTCCAT |
| | DNA) | GAAGCTCTCCTGTGCAGCCTCAGGATTCACTTTCAGTAACTATTACATGGCCT |
| | | GGGTCCGCCAGGCTCCAAGGAAGGGTCTGGAGTGGGTCGCATCCATTACTACT |
| | | GGTGGAACTAACACTTACTATCGAGACTCCGTGAGGGGCCGATTCACTATCTC |
| | | CAGAGATAATGCAAAAAGCACCCTATACCTGCAAATGGACAGTCTGAGGTCTG |
| | | AGGAAACGGCCACTTATTACTGTGCAAGACATTCGGGGGGTTTAATTCGGGCC |
| | | CGGGGCCACTACTTTCATTACTGGGGCCAAGGAGTCATGGTCACAGTCTCCTC |
| | | A |
| 398 | ZNRF3-90 (VL | GACATCCAGATGACACAGTCTCCAGCTTCCCTGTCTGCATCTCTTGGAGAAAT |
| | DNA) | TGTCTCCATCGAATGTCTAGCAAGTGAGGGCATTTCCAATGATTTAGCGTGGT |
| | | ATCAGCAGAAGTCAGGGAAATCTCCTCAGGTCCTGGTCTATGCTGCAAGTAGG |
| | | TTGCAAGACGGGGTCCCATCACGGTTCAGTGGCAGTGGATCTGGCACACGGTA |
| | | TTCTCTCAAGATCAGCGGCATGCAACCTGAAGATGAAGCAGATTATTTCTGTC |
| | | AACAGAGTTACACGTATCCGTACACGTTTGGAGCTGGGACCAAGCTGGAACTG |
| | | AAA |
| 399 | ZNRF3-90 (VH | EVQLVESGGGLVQPGRSMKLSCAASGFTFSNYYMAWVRQAPRKGLEWVASITT |
| | prot) | GGTNTYYRDSVRGRFTISRDNAKSTLYLQMDSLRSEETATYYCARHSGGLIRA |
| | | RGHYFHYWGQGVMVTVSS |
| 400 | ZNRF3-90 (VL | DIQMTQSPASLSASLGEIVSIECLASEGISNDLAWYQQKSGKSPQVLVYAASR |
| | prot) | LQDGVPSRFSGSGSGTRYSLKISGMQPEDEADYFCQQSYTYPYTFGAGTKLEL |
| | | K |
| 401 | RNF43-1 (VH | QSVEESGGRLVTPGTPLILTCTVSGIDLSRYNMNWVRQAPGKGLEYIGYIYYG |
| | prot) | TGTTYYASWVKGRFTISKTSTTVDLKITSPTTEDTATYFCASGDFWGPGTLVT |
| | | VSS |
| 402 | RNF43-1 (VL | DIVMTQTPASVEAAVGGTVTINCQASQSISSYLAWYQQKPGQPPKLLIYYASN |
| | prot) | LESGVPSRFKGSGSGTQFTLTISDLECADAATYYCQAYYSSITGWAFGGGTEV |
| | | VVK |
| 403 | RNF43-24 (VH | QEQLVESGGGLVQPGESLTLSCKASGFSFSTGYDMCWVRQAPGKGLEWIGCIG |
| | prot) | TSSGDTGYASWAKGRFTISKASSTTVTLQMTSLTAADTATYFCARGESLWGPG |
| | | TLVTVSS |
| 404 | RNF43-24 (VL | AQVLTQTASPVSAAVGGTVTINCQSSQSVYSNNRLGWYQQKPGQPPRLLIYQT |
| | prot) | SKLASGVPSRFRGSGSGTQFTLTISDLECDDAATYYCLGIYDCSSADCTAFGG |
| | | GTEVVVK |
| 405 | RNF43-8 (VH | QSVEESGGRLVTPGTPLTLTCTVSGFSLSSYAMAWVRQAPGEGLEWIGTINRA |
| | prot) | GNTYYASWAKGRFTISKTSTTVDLKVTSPTTEDTATYFCARSLYSTNNLWGPG |
| | | TLVTVSS |
| 406 | RNF43-8 (VL | AIVMTQTPSSKSVPVGGTVTISCQASESLYDNDDLSWFQQKPGQPPKLLIYQA |
| | prot) | SSLASGVPSRFKGSGSGTQFTLTISDVVCGDAATYYCAGYAGSSTDGVAFGGG |
| | | TEVVVK |
| 407 | RNF43-12 (VH | QEQLVESGGGLVKPGASLTLTCKASGFSLSNGDVMCWVRQAPGKGLEWIGCIG |
| | prot) | ISNNDRAAYASWAKGRFTISKTSSTTVTLQTTSLTAADSATYFCARYLASEYY |
| | | NLWGPGTLVTGSS |
| 408 | RNF43-12 (VL | AYDMTQTPASVEAAVGGTITINCQASESISSWLAWYQQKPGQPPKLLIYKAST |
| | prot) | LASGVSSRFEGSGSGTEFTLTISDLECADAATYYCQQGASWSNVENTFGGGTE |
| | | VVVK |
| 409 | RNF43-20 (VH | REQLVESGGGLVQPEGSLTLTCKASGFDFSSNVMCWVRQAPGKGLEWIGCIYT |
| | prot) | GSGSTAYASWAKGRFTITRSTSLNTVTLQMTSLTAADTATYFCARYVSGGEYY |
| | | NLWGPGTLVTVSS |
| 410 | RNF43-20 (VL | ALVMTQTPASVEAAVGGTVTIKCQASQSISSYLNWYQQKPGQPPKLLIYKAST |
| | prot) | LASGVSSRFKGSGSGTQFTLTISDLECADAATYYCQQGSSWNSIENIFGGGTE |
| | | VVVK |
| 411 | RNF43-11 (VH | QEQLVESGGGLVQPEGSLTLTCKASGFDESSNVMCWVRQAPGKGLEWIGCIYT |
| | prot) | GSSGSTYYASWAKGRFTITRSTSLNTVTLQMTSLTAADTATYFCARYVSGGEY |
| | | YNLWGPGTLVTVSS |
| 412 | RNF43-11 (VL | ALVMTQTPASVEAAVGGTVTINCQASESISNWLAWYQQKPGQPPKLLIYKAST |
| | prot) | LASGVSSRFKGSGSGTQFTLTISDLECADAATYYCQQGSSWINIEDVFGGGTE |
| | | VVVK |
| 413 | RNF43-23 (VH | QEQVVESGGGLVQPEGSLTLTCKGSGFDFSGNVLCWVRQAPGKGLEWIGCIAT |
| | prot) | GIGSTAYASWAKGRFTITRGTSLNTVDLKMTSLTAADTATYFCARYVSGSEYY |
| | | NEWGPGTLVTVSS |
| 414 | RNF43-23 (VL | AYDMTQTPASVEVAVGGTVTIKCQASQSISSFLAWYQQKPGQPPMLLIYKAST |
| | prot) | LASGVPSRFKGSGSGTQFTLTISGVECADAATYYCQQGYSWNGVDNTFGGGTE |
| | | VVVK |
| 415 | RNF43-6 (VH | QSLEESGGRLVTPGTPLTLTCTVSGFSLSAFGMNWVRQAPGKGLEWIGYISAG |
| | prot) | GSAVYPNWAEGRFTISKASTTVTLKMTSLTTEDTATYFCVRGGYVGSTNSLWG |
| | | PGTLVTVSS |
| 416 | RNF43-6 (VL | AAVLTQTPSPVSAAVGGTVSISCQSSKSVHNNNWLAWYQQKPGQPPKLLIYGA |
| | prot) | STLASGVPSRFKGSGSGTQFTLTISDVQCDDAATYYCSGGYSSIIDNGFGGGT |
| | | DVVVE |
| 417 | RNF43-5 (VH | QSVEESGGRLVTPGTPLTLTCTVSGFSLNTYAMGWVRQAPGKGLEWIGTIGSS |
| | prot) | GSTYYATWAKGRFTVSKTSTTVDLKITSPTTEDTATYFCVRGADGLNYYPIWG |
| | | PGTLVTVSL |
| 418 | RNF43-5 (VL | AQVLTQTPSSVSAAVGGTVTINCQASQSVYNNKNLAWYQQKPGQPPKLLIYDA |
| | prot) | SDLASGVSSRFKGSGSGTQFTLTISGVQCDDAATYYCQGEFNCDKGDCNAFGG |
| | | GTEVVVK |
| 419 | RNF43-15 (VH | QEQLEESGGDLVKPGASLTLTCTASRFSFSNDDVICWVRQAPGKGLEWIGCIA |
| | prot) | TINGNTAYATWAKGRFTSSKTSSTTVTLQMTRLTAADTATYFCARWDTGSEHV |
| | | TLWGPGTLVTVSS |
| 420 | RNF43-15 (VL | AYDMTQTPASVEVAVGGTVTIKCQASQSISSYLAWYQQKPGQPPKPLIYEASK |
| | prot) | LASGVPSRFSGSGSGTQFTLTISGVQCADAATYYCQQGASWSNVDNVFGGGTE |
| | | VVVK |
| 421 | RNF43-2 (VH | QSLEESGGDLVKPGASLTLTCTASGFSFSSSYYMCWVRQAPGKGLELIGCGFT |
| | prot) | GRHYTYYASWAKGRFTISKSSSTTVTLQMTSLTAADTATYFCARKSGSDYDYF |
| | | DLWGPGTLVTVSS |
| 422 | RNF43-2 (VL | AYDMTQTPASVEAVVGGTVTIKCQASQSINSWLSWYQQKPGQRPKLLIYRAST |
| | prot) | LASGVSSRFKGSGSGTEFTLTISDLECADAATYYCQQGYVSSNINNVFGGGTE |
| | | VVVK |
| 423 | RNF43-16 (VH | QSVEESGGRLVTPGTPLTLTCTVSGFFLSSYYMSWVRQAPGKGLEWIGYIYAG |
| | prot) | SGSTYYASWAKGRFTISKTSTTVDLKITSPTTEDTATYFCVRDINGGDYGMDP |
| | | WGPGTLVTVSS |
| 424 | RNF43-16 (VL | DIVMTQTPASVSAAVGGTVTIKCQASESIDSGLAWYQQKPGQRPKLLIYAASN |
| | prot) | LASGVSSRFKGSGSGTEYSLTISGVQCDDAATYYCQCTYGSVTSNTYGIAFGG |
| | | GTEVVVK |
| 425 | RNF43-14 (VH | QSVEESGGRLVTPGTPLTLTCTVSGIDLSSYAMGWVRQAPGKGLEYIGFIYAS |
| | prot) | GSTYYASWAKGRFTISKTSTTVDLKITSPTTEDTATYFCARDSSETINYENIW |
| | | GPGTLVTVSL |
| 426 | RNF43-14 (VL | NIVMTQTPSPVSGAVGGTVTIKCQASQSISNWLAWYQQKPGQPPKVLIYRAST |
| | prot) | LASGVSSRFKGSGSGTEFTLTISDLECADAATYYCQQGYYISYLDNAFGGGTE |
| | | VVVK |
| 427 | RNF43-17 (VH | QSVEESGGRLVTPGTPLTLTCTGSGFSLSNYAMGWVRQAPGKGLEYIGIIYAS |
| | prot) | GSTWYASWAKGRFTISKTSTTVDLKITSPATEDTATYFCCRDATTTYDRLDLW |
| | | GQGTLITVSS |
| 428 | RNF43-17 (VL | AFELTQTPSSVEAAVGGTVTIKCQASQSISKWLAWYQQKPGQPPKLLIYRAST |
| | prot) | LPSGVPSRFKGSGSGTEFTLTISGVECADAATYYCQQGYSVTNLDEAFGGGTE |
| | | VVVK |
| 429 | RNF43-22 (VH | QEQLEESGGDLVKPGASLTLTCTASGFSVSNSYYMCWVRQAPGKGLEWIACIN |
| | prot) | TGSSGSTYYASWAKGRFTISKTSSTTVTLQMTTLTAADTATYFCARGYAGYAG |
| | | YGQFNLWGPGTLVTVSS |
| 430 | RNF43-22 (VL | AYDMTQTPASVEVAVGGTVTIKCQASQSISSYLAWYQQKPGQRPKVLIYDVSK |
| | prot) | LASGVPSRFKGSGSGTQFTLTISGVECADAATYYCQQGYSMSNVDNTFGGGTE |
| | | VVVK |
| 431 | RNF43-9 (VH | QSLEESGGDLVQPEGSLTLTCTASGFSFSSSCYMCWVRQAPGKGLEWIACINT |
| | prot) | GSSGSTYYASWAKGRFTISKTSSTTVTLQMTILTAADTATYFCARGYAGYTGY |
| | | GQFNLWGPGTLVTVSS |
| 432 | RNF43-9 (VL | QIVMTQTPASVEAAVGGTVTIKCQASQSISNYLAWYQQKPGQRPKLLIYDASK |
| | prot) | LASGVPSRFKGSGSGTQFTLTISGVECADAATYYCQQGYTMNNVDNAFGGGTE |
| | | VVVK |
| 433 | RNF43-21 (VH | QSLEESGGDLVKPGASLTLTCTASGFSFSSSYYMCWVRQAPGKGLEWIACIYA |
| | prot) | GSSGSTYYASWAKGRFTVSKTSSTTVTLQMTSLTAADTATYFCARYTYGSGSD |
| | | SYFNLWGPGTLVTVSS |
| 434 | RNF43-21 (VL | AYDMTQTPASVEAAVGGTVTIQCQASQSIGSYLAWYQQKPGQPPKLLIYRAST |
| | prot) | LSSGVPSRFKGSGSGTQFTLTISDLECADAATYYCQQGYSITNLDNGFGGGTE |
| | | VVVK |
| 435 | RNF43-13 (VH | QSLEESGGGLVKPGGTLTLTCKASGIDFSGYYYMCWVRQAPGKGLEWIACIYT |
| | prot) | TSGSAWYASWAKGRFTISITTSLNTVTLQMTSLTATDTATYFCVRYANYGGYN |
| | | YPEFGVLWGPGTLVTVSS |
| 436 | RNF43-13 (VL | AYDMTQTPASVEVAVGATVTIKCQASEDIYNLLAWYQQKPGQPPKLLIYRASK |
| | prot) | LASGVPSRFSGSGSGTEFTLTISSVQCDDAATYYCQQGYSISNVENPFGGGTE |
| | | VVVK |
| 437 | RNF43-19 (VH | QQQLEESGGGLVKPGGTLTLTCKASGIDFSTYYYMCWVRQAPGKGLEWIACIY |
| | prot) | TGDGNTYYASWAKGRFTISKTSSTTVTLQMTSLTAADTATYFCARDLAGSSYY |
| | | SGYYFKLWGPGTLVTVSS |
| 438 | RNF43-19 (VL | DVVMTQTPASVSEPVGGTVTIKCQASQSIGSNLAWYQQKPGQPPKLLIYSVSN |
| | prot) | LASGVSSRFKGSRSGTEYTLTISDLECADAATYYSQSGYYGSTYAFGGGTEVV |
| | | VK |
| 439 | ZNRF3-331 (VH | QSVEESGGRLVTPGTPLTLTCTVSGFSLSSYAMGWVRQAPGKGLEWIGIIYAG |
| | prot) | DTTYYASWAKGRFTISETSTTVDLKITSPTTEDTATYFCARKSDDDSDSYLLW |
| | | GPGTLVTVSS |
| 440 | ZNRF3-331 (VL | AYDMTQTPASVEVAVGGTVTIKCQASQSISSYLNWYQQKRGQPPKLLIYDAST |
| | prot) | LASGVPSRFSGSGSGTEFTLTISGVQCDDAATYYCQQGWSSTNIDNIFGGGTE |
| | | VVVK |
| 441 | ZNRF3-333 (VH | QSLEESGGGLVQPEGSLTLTCTASGIDFSARSYMCWVRQAPGKGLEWVACIYT |
| | prot) | GSSGSTYYANWAKGRFTISKTSSTTVTLRMTSLTAADTASYFCARGAYVGYGF |
| | | AAYFTLWGPGTLVTVSS |
| 442 | ZNRF3-333 (VL | DIVMTQTPASVEAAVGGTVTINCQASQSISSYLAWYQQKPGQPPKLLIYYASN |
| | prot) | LESGVPSRFKGSGSGTQFTLTISDLECADAATYYCQAYYSSITGWAFGGGTEV |
| | | VVK |
| 443 | ZNRF3-332 (VH | QSLEESGGGLVQPEGSLTLTCTASGIDESTRNYMCWVRQAPGKGLEWVGCIYT |
| | prot) | SSGGTYYASWVNGRFTISKTSSTTVTLRMTSLTAADTATYFCARGGYVGYGYA |
| | | TYFNLWGPGTLVAVSS |
| 444 | ZNRF3-332 (VL | DVVMTQTPASVSEPVGGTVTIKCQASQSISSYLSWYQQKPGQPPKLLIYYVSN |
| | prot) | LESGVPSRFKGSGSGTEFTLTISDLECADAATYYCQNYYSSSSGWAFGGGTEV |
| | | VVK |
| 445 | Human RNF13 | MLLSIGMLMLSATQVYTILTVQLFAFLNLLPVEAGKYALADASLKMADPNRER |
| | (043567; | GKDLPVLGSDILAYNFENASQTFDDLPARFGYRLPAEGLKGFLINSKPENACE |
| | UNQ16056) | PIVPPPVKDNSSGTFIVLIRRLDCNFDIKVLNAQRAGYKAAIVHNVDSDDLIS |
| | | MGSNDIEVLKKIDIPSVFIGESSANSLKDEFTYEKGGHLILVPEFSLPLEYYL |
| | | IPFLIIVGICLILIVIFMITKFVQDRHRARRNRLRKDQLKKLPVHKFKKGDEY |
| | | DVCAICLDEYEDGDKLRILPCSHAYHCKCVDPWLTKTKKTCPVCKQKVVPSQG |
| | | DSDSDTDSSQEENEVTEHTPLLRPLASVSAQSFGALSESRSHQNMTESSDYEE |
| | | DDNEDTDSSDAENEINEHDVVVQLQPNGERDYNIANTV |
| 446 | Human RNF43 | MSGGHQLQLAALWPWLLMATLQAGKYALADASLKMADPNRFRGKDLPVLGSGF |
| | (Q68DV7; | GRTGLVLAAAVESERSAEQKAIIRVIPLKMDPTGKLNLTLEGVFAGVAEITPA |
| | UNQ9671) | EGKLMQSHPLYLCNASDDDNLEPGFISIVKLESPRRAPRPCLSLASKARMAGE |
| | | RGASAVLFDITEDRAAAEQLQQPLGLTWPVVLIWGNDAEKLMEFVYKNQKAHV |
| | | RIELKEPPAWPDYDVWILMTVVGTIFVIILASVLRIRCRPRHSRPDPLQQRTA |
| | | WAISQLATRRYQASCRQARGEWPDSGSSCSSAPVCAICLEEFSEGQELRVISC |
| | | LHEFHRNCVDPWLHQHRTCPLCMFNITEGDSFSQSLGPSRSYQEPGRRLHLIR |
| | | QHPGHAHYHLPAAYLLGPSRSAVARPPRPGPFLPSQEPGMGPRHHRFPRAAHP |
| | | RAPGEQQRLAGAQHPYAQGWGLSHLQSTSQHPAACPVPLRRARPPDSSGSGES |
| | | YCTERSGYLADGPASDSSSGPCHGSSSDSVVNCTDISLQGVHGSSSTFCSSLS |
| | | SDFDPLVYCSPKGDPQRVDMQPSVTSRPRSLDSVVPTGETQVSSHVHYHRHRH |
| | | HHYKKRFQWHGRKPGPETGVPQSRPPIPRTQPQPEPPSPDQQVTRSNSAAPSG |
| | | RLSNPQCPRALPEPAPGPVDASSICPSTSSLENLQKSSLSARHPQRKRRGGPS |
| | | EPTPGSRPQDATVHPACQIFPHYTPSVAYPWSPEAHPLICGPPGLDKRLLPET |
| | | PGPCYSNSQPVWLCLTPRQPLEPHPPGEGPSEWSSDTAEGRPCPYPHCQVLSA |
| | | QPGSEEELEELCEQAV |
| 447 | Human SYVN1 | MFRTAVMMAASLALTGAVVAHAGKYALADASLKMADPNRFRGKDLPVLGSYYL |
| | (Q86TM6; | KHQFYPTVVYLTKSSPSMAVLYIQAFVLVFLLGKVMGKVFFGQLRAAEMEHLL |
| | UNQ22255) | ERSWYAVTETCLAFTVFRDDFSPRFVALFTLLLFLKCFHWLAEDRVDEMERSP |
| | | NISWLFHCRIVSLMFLLGILDFLFVSHAYHSILTRGASVQLVFGFEYAILMTM |
| | | VLTIFIKYVLHSVDLQSENPWDNKAVYMLYTELFTGFIKVLLYMAFMTIMIKV |
| | | HTFPLFAIRPMYLAMRQFKKAVTDAIMSRRAIRNMNTLYPDATPEELQAMDNV |
| | | CIICREEMVTGAKRLPCNHIFHTSCLRSWFQRQQTCPTCRMDVLRASLPAQSP |
| | | PPPEPADQGPPPAPHPPPLLPQPPNFPQGLLPPFPPGMFPLWPPMGPFPPVPP |
| | | PPSSGEAVAPPSTSAAALSRPSGAATTTAAGTSATAASATASGPGSGSAPEAG |
| | | PAPGFPFPPPWMGMPLPPPFAFPPMPVPPAGFAGLTPEELRALEGHERQHLEA |
| | | RLQSLRNIHTLLDAAMLQINQYLTVLASLGPPRPATSVNSTEETATTVVAAAS |
| | | STSIPSSEATTPTPGASPPAPEMERPPAPESVGTEEMPEDGEPDAAELRRRRL |
| | | QKLESPVAH |
| 448 | Human RNF130 | MSCAGRAGPARLAALALLTCSLWPARAGKYALADASLKMADPNRFRGKDLPVL |
| | (Q86XS8; | GSDNASQEYYTALINVTVQEPGRGAPLTFRIDRGRYGLDSPKAEVRGQVLAPL |
| | UNQ4694) | PLHGVADHLGCDPQTRFFVPPNIKQWIALLQRGNCTFKEKISRAAFHNAVAVV |
| | | IYNNKSKEEPVTMTHPGTGDIIAVMITELRGKDILSYLEKNISVQMTIAVGTR |
| | | MPPKNFSRGSLVFVSISFIVLMIISSAWLIFYFIQKIRYTNARDRNQRRLGDA |
| | | AKKAISKLTTRTVKKGDKETDPDFDHCAVCIESYKQNDVVRILPCKHVFHKSC |
| | | VDPWLSEHCTCPMCKLNILKALGIVPNLPCTDNVAFDMERLTRTQAVNRRSAL |
| | | GDLAGDNSLGLEPLRTSGISPLPQDGELTPRTGEINIAVTKEWFIIASFGLLS |
| | | ALTLCYMIIRATASLNANEVEWE |
| 449 | Human RNF148 | MSFLRITPSTHSSVSSGLLRLSIFLLLSFPDSNGGKYALADASLKMADPNRER |
| | (Q8N7C7; | GKDLPVLGSKAIWTAHLNITFQVGNEITSELGESGVFGNHSPLERVSGVVALP |
| | UNQ34666) | EGWNQNACHPLTNFSRPKQADSWLALIERGGCTFTHKINVAAEKGANGVIIYN |
| | | YQGTGSKVFPMSHQGTENIVAVMISNLKGMEILHSIQKGVYVTVIIEVGRMHM |
| | | QWVSHYIMYLFTFLAATIAYFYLDCVWRLTPRVPNSFTRRRSQIKTDVKKAID |
| | | QLQLRVLKEGDEELDLNEDNCVVCFDTYKPQDVVRILTCKHFFHKACIDPWLL |
| | | AHRTCPMCKCDILKT |
| 450 | Human RNF149 | MAWRRREASVGARGVLALALLALALCVPGARGGKYALADASLKMADPNRFRGK |
| | (Q8NC42; | DLPVLGSRALEWFSAVVNIEYVDPQTNLTVWSVSESGRFGDSSPKEGAHGLVG |
| | UNQ4467) | VPWAPGGDLEGCAPDTRFFVPEPGGRGAAPWVALVARGGCTFKDKVLVAARRN |
| | | ASAVVLYNEERYGNITLPMSHAGTGNIVVIMISYPKGREILELVQKGIPVTMT |
| | | IGVGTRHVQEFISGQSVVFVAIAFITMMIISLAWLIFYYIQRFLYTGSQIGSQ |
| | | SHRKETKKVIGQLLLHTVKHGEKGIDVDAENCAVCIENFKVKDIIRILPCKHI |
| | | FHRICIDPWLLDHRTCPMCKLDVIKALGYWGEPGDVQEMPAPESPPGRDPAAN |
| | | LSLALPDDDGSDDSSPPSASPAESEPQCDPSFKGDAGENTALLEAGRSDSRHG |
| | | GPIS |
| 451 | Human LNX1- | MKALLLLVLPWLSPAGKYALADASLKMADPNRFRGKDLPVLGSNYIDNVGNLH |
| | isoform2 | FLYSELCKGASHYGLTKDRKRRSQDGCPDGCASLTATAPSPEVSAAATISLMT |
| | (Q8TBB; UNQ | DEPGLDNPAYVSSAEDGQPAISPVDSGRSNRTRARPFERSTIRSRSFKKINRA |
| | 574) | LSVLRRTKSGSAVANHADQGRENSENTTAPEVFPRLYHLIPDGEITSIKINRV |
| | | DPSESLSIRLVGGSETPLVHIIIQHIYRDGVIARDGRLLPGDIILKVNGMDIS |
| | | NVPHNYAVRLLRQPCQVLWLTVMREQKFRSRNNGQAPDAYRPRDDSFHVILNK |
| | | SSPEEQLGIKLVRKVDEPGVFIFNVLDGGVAYRHGQLEENDRVLAINGHDLRY |
| | | GSPESAAHLIQASERRVHLVVSRQVRQRSPDIFQEAGWNSNGSWSPGPGERSN |
| | | TPKPLHPTITCHEKVVNIQKDPGESLGMTVAGGASHREWDLPIYVISVEPGGV |
| | | ISRDGRIKTGDILLNVDGVELTEVSRSEAVALLKRTSSSIVLKALEVKEYEPQ |
| | | EDCSSPAALDSNHNMAPPSDWSPSWVMWLELPRCLYNCKDIVLRRNTAGSLGF |
| | | CIVGGYEEYNGNKPFFIKSIVEGTPAYNDGRIRCGDILLAVNGRSTSGMIHAC |
| | | LARLLKELKGRITLTIVSWPGTFL |
| 452 | Human RNF128 | MGPPPGAGVSCRGGCGFSRLLAWCFLLALSPQAPGSRGGKYALADASLKMADP |
| | (Q8TEB7; | NRFRGKDLPVLGSAEAVWTAYLNVSWRVPHTGVNRTVWELSEEGVYGQDSPLE |
| | UNQ8231) | PVAGVLVPPDGPGALNACNPHTNFTVPTVWGSTVQVSWLALIQRGGGCTFADK |
| | | IHLAYERGASGAVIFNFPGTRNEVIPMSHPGAVDIVAIMIGNLKGTKILQSIQ |
| | | RGIQVTMVIEVGKKHGPWVNHYSIFFVSVSFFIITAATVGYFIFYSARRLRNA |
| | | RAQSRKQRQLKADAKKAIGRLQLRTLKQGDKEIGPDGDSCAVCIELYKPNDLV |
| | | RILTCNHIFHKTCVDPWLLEHRTCPMCKCDILKALGIEVDVEDGSVSLQVPVS |
| | | NEISNSASSHEEDNRSETASSGYASVQGTDEPPLEEHVQSTNESLQLVNHEAN |
| | | SVAVDVIPHVDNPTFEEDETPNQETAVREIKS |
| 453 | Human (RNF133 | MHLLKVGTWRNNTASSWLMKFSVLWLVSQNCCRAGKYALADASLKMADPNRER |
| | (Q8WVZ7; | GKDLPVLGSSVVWMAYMNISFHVGNHVLSELGETGVFGRSSTLKRVAGVIVPP |
| | UNQ34667) | EGKIQNACNPNTIFSRSKYSETWLALIERGGCTFTQKIKVATEKGASGVIIYN |
| | | VPGTGNQVFPMFHQAFEDVVVVMIGNLKGTEIFHLIKKGVLITAVVEVGRKHI |
| | | IWMNHYLVSFVIVTTATLAYFIFYHIHRLCLARIQNRRWQRLTTDLQNTFGQL |
| | | QLRVVKEGDEEINPNGDSCVICFERYKPNDIVRILTCKHFFHKNCIDPWILPH |
| | | GTCPICKCDILKVLGIQVVVENGTEPLQVLMSNELPETLSPSEEETNNEVSPA |
| | | GTSDKVIHVEENPTSQNNDIQPHSVVEDVHPSP |
| 454 | Human ZNRF4 | MPLCRPEHLMPRASRVPVAASLPLSHAGKYALADASLKMADPNRFRGKDLPVL |
| | (Q8WWF5; | GSVIPTQLPSRPGHRPPGRPRRCPKASCLPPPVGPSSTQTAKRVTMGWPRPGR |
| | UNQ12510) | ALVAVKALLVLSLLQVPAQAVVRAVLEDNSSSVDFADLPALFGVPLAPEGIRG |
| | | YLMEVKPANACHPIEAPRLGNRSLGAIVLIRRYDCTFDLKVLNAQRAGFEAAI |
| | | VHNVHSDDLVSMTHVYEDLRGQIAIPSVFVSEAASQDLRVILGCNKSAHALLL |
| | | PDDPPCHDLGCHPVLTVSWVLGCTLALVVSAFFVLNHLWLWAQACCSHRRPVK |
| | | TSTCQKAQVRTFTWHNDLCAICLDEYEEGDQLKILPCSHTYHCKCIDPWFSQA |
| | | PRRSCPVCKQSVAATEDSFDSTTYSFRDEDPSLPGHRPPIWAIQVQLRSRRLE |
| | | LLGRASPHCHCSTTSLEAEYTTVSSAPPEAPGQ |
| 455 | Human RSPRY1 | MIVFGWAVFLASRSLGGKYALADASLKMADPNRFRGKDLPVLGSQGLLLTLEE |
| | (Q96DX4; | HIAHFLGTGGAATTMGNSCICRDDSGTDDSVDTQQQQAENSAVPTADTRSQPR |
| | UNQ328) | DPVRPPRRGRGPHEPRRKKQNVDGLVLDTLAVIRTLVDNDQEPPYSMITLHEM |
| | | AETDEGWLDVVQSLIRVIPLEDPLGPAVITLLLDECPLPTKDALQKLTEILNL |
| | | NGEVACQDSSHPAKHRNTSAVLGCLAEKLAGPASIGLLSPGILEYLLQCLKLQ |
| | | SHPTVMLFALIALEKFAQTSENKLTISESSISDRLVTLESWANDPDYLKRQVG |
| | | FCAQWSLDNLFLKEGRQLTYEKVNLSSIRAMLNSNDVSEYLKISPHGLEARCD |
| | | ASSFESVRCTFCVDAGVWYYEVTVVTSGVMQIGWATRDSKELNHEGYGIGDDE |
| | | YSCAYDGCRQLIWYNARSKPHIHPCWKEGDTVGFLLDLNEKQMIFFLNGNQLP |
| | | PEKQVFSSTVSGFFAAASFMSYQQCEFNFGAKPFKYPPSMKFSTENDYAFLTA |
| | | EEKIILPRHRRLALLKQVSIRENCCSLCCDEVADTQLKPCGHSDLCMDCALQL |
| | | ETCPLCRKEIVSRIRQISHIS |
| 456 | Human TRIM7 | MTQATGQMLCLGKYALADASLKMADPNRFRGKDLPVLGSHVQVPLQLLLLGQK |
| | isoform 3 | QMAAEQEKVGAEFQALRAFLVEQEGRLLGRLEELSREVAQKQNENLAQLGVEI |
| | (Q9C029-4; | TQLSKLSSQIQETAQKPDLDFLQEFKSTLSRCSNVPGPKPTTVSSEMKNKVWN |
| | UNQ 20983) | VSLKTFVLKGMLKKFKEDLRGELEKEEKVELTLDPDTANPRLILSLDLKGVRL |
| | | GERAQDLPNHPCRFDTNTRVLASCGFSSGRHHWEVEVGSKDGWAFGVARESVR |
| | | RKGLTPFTPEEGVWALQLNGGQYWAVTSPERSPLSCGHLSRVRVALDLEVGAV |
| | | SFYAVEDMRHLYTFRVNFQERVFPLFSVCSTGTYLRIWP |
| 457 | Human RNF167 | MHPAAFPLPVVVAAVLWGAAPTRGGKYALADASLKMADPNRFRGKDLPVLGSL |
| | (Q9H6Y7; | IRATSDHNASMDFADLPALFGATLSQEGLQGFLVEAHPDNACSPIAPPPPAPV |
| | UNQ16055) | NGSVFIALLRRFDCNFDLKVLNAQKAGYGAAVVHNVNSNELLNMVWNSEEIQQ |
| | | QIWIPSVFIGERSSEYLRALFVYEKGARVLLVPDNTFPLGYYLIPFTGIVGLL |
| | | VLAMGAVMIARCIQHRKRLQRNRLTKEQLKQIPTHDYQKGDQYDVCAICLDEY |
| | | EDGDKLRVLPCAHAYHSRCVDPWLTQTRKTCPICKQPVHRGPGDEDQEEETQG |
| | | QEEGDEGEPRDHPASERTPLLGSSPTLPTSFGSLAPAPLVFPGPSTDPPLSPP |
| | | SSPVILV |
| 458 | Human RNF150 | MAMSLIQACCSLALSTWLLSFCFVHLLCLDFTVAGKYALADASLKMADPNRER |
| | (Q9ULK6; | GKDLPVLGSEKEEWYTAFVNITYAEPAPDPGAGAAGGGGAELHTEKTECGRYG |
| | UNQ11523) | EHSPKQDARGEVVMASSAHDRLACDPNTKFAAPTRGKNWIALIPKGNCTYRDK |
| | | IRNAFLQNASAVVIFNVGSNTNETITMPHAGVEDIVAIMIPEPKGKEIVSLLE |
| | | RNITVTMYITIGTRNLQKYVSRTSVVFVSISFIVLMIISLAWLVFYYIQRFRY |
| | | ANARDRNQRRLGDAAKKAISKLQIRTIKKGDKETESDFDNCAVCIEGYKPNDV |
| | | VRILPCRHLFHKSCVDPWLLDHRTCPMCKMNILKALGIPPNADCMDDLPTDFE |
| | | GSLGGPPTNQITGASDTTVNESSVTLDPAVRTVGALQVVQDTDPIPQEGDVIF |
| | | TTNSEQEPAVSSDSDISLIMAMEVGLSDVELSTDQDCEEVKS |
| 459 | Human ZNRF3 | MRPRSGGRPGATGRRRRRLRRRPRGLRCSRLPPPPPLPLLLGLLLAAAGPGAA |
| | (Q9ULT6; | RAGKYALADASLKMADPNRFRGKDLPVLGSKETAFVEVVLFESSPSGDYTTYT |
| | UNQ28198) | TGLTGRESRAGATLSAEGEIVQMHPLGLCNNNDEEDLYEYGWVGVVKLEQPEL |
| | | DPKPCLTVLGKAKRAVQRGATAVIFDVSENPEAIDQLNQGSEDPLKRPVVYVK |
| | | GADAIKLMNIVNKQKVARARIQHRPPRQPTEYFDMGIFLAFFVVVSLVCLILL |
| | | VKIKLKQRRSQNSMNRLAVQALEKMETRKENSKSKGRREGSCGALDTLSSSST |
| | | SDCAICLEKYIDGEELRVIPCTHRFHRKCVDPWLLQHHTCPHCRHNIIEQKGN |
| | | PSAVCVETSNLSRGRQQRVTLPVHYPGRVHRTNAIPAYPTRTSMDSHGNPVTL |
| | | LTMDRHGEQSLYSPQTPAYIRSYPPLHLDHSLAAHRCGLEHRAYSPAHPERRP |
| | | KLSGRSFSKAACFSQYETMYQHYYFQGLSYPEQEGQSPPSLAPRGPARAFPPS |
| | | GSGSLLFPTVVHVAPPSHLESGSTSSFSCYHGHRSVCSGYLADCPGSDSSSSS |
| | | SSGQCHCSSSDSVVDCTEVSNQGVYGSCSTFRSSLSSDYDPFIYRSRSPCRAS |
| | | EAGGSGSSGRGPALCFEGSPPPEELPAVHSHGAGRGEPWPGPASPSGDQVSTC |
| | | SLEMNYSSNSSLEHRGPNSSTSEVGLEASPGAAPDLRRTWKGGHELPSCACCC |
| | | EPQPSPAGPSAGAAGSSTLFLGPHLYEGSGPAGGEPQSGSSQGLYGLHPDHLP |
| | | RTDGVKYEGLPCCFYEEKQVARGGGGGSGCYTEDYSVSVQYTLTEEPPPGCYP |
| | | GARDLSQRIPIIPEDVDCDLGLPSDCQGTHSLGSWGGTRGPDTPRPHRGLGAT |
| | | REEERALCCQARALLRPGCPPEEAGAVRANFPSALQDTQESSTTATEAAGPRS |
| | | HSADSSSPGA |

1. A multispecific binding protein that binds to at least a first cell surface target protein and a second cell surface protein, wherein the first cell surface target protein is a transmembrane E3 ubiquitin ligase.

2. The multispecific binding protein of claim 1, wherein (a) the multispecific binding protein reduces the level of the second cell surface protein on the surface of a cell compared to the level observed in the absence of the multispecific binding protein, (b) the multispecific binding protein reduces the level of the second cell surface protein on the surface of a cell in vitro compared to the level observed in the absence of the multispecific binding protein, (c) the multispecific binding protein reduces the level of the second cell surface protein on the surface of a cell as determined by flow cytometry or by luminescense assay, and/or (d) the multispecific binding protein reduces the level of the second cell surface protein on the surface of a cell in vivo compared to the level observed in the absence of the multispecific binding protein.

3. (canceled)

4. (canceled)

5. (canceled)

6. The multispecific binding protein of claim 1, wherein the multispecific binding protein is a multispecific antibody, optionally wherein the multispecific antibody is a bispecific antibody, a trispecific antibody, a 1+1 FabIgG, a 1+1 FvIgG, a 2+1 FvIgG, 2+1 FabIgG, a one-armed FvIgG, or a one-armed FabIgG.

7. (canceled)

8. (canceled)

9. (canceled)

10. (canceled)

11. The multispecific binding protein of claim 1, wherein the transmembrane E3 ubiquitin ligase is RNF43, ZNRF3, RNF13, RNF128, RNF130, RNF133, RNF148, RNF149, RNF150, RNF167, ZNRF4, RSPRY1, SYVN1, LNX1 isoform 2, or TRIM7 isoform 3.

12. (canceled)

13. (canceled)

14. (canceled)

15. (canceled)

16. The multispecific binding protein of claim 1, wherein the protein binds to RNF43, ZNRF3, or both RNF43 and ZNRF3, optionally wherein the protein does not block binding between RNF43 and/or ZNRF3 and FZD and/or LRP6.

17. (canceled)

18. (canceled)

19. (canceled)

20. (canceled)

21. (canceled)

22. The multispecific binding protein of any one of claim 1, wherein the protein binds to RNF43, ZNRF3, or both RNF43 and ZNRF3, and comprises a heavy chain variable domain (VH) comprising (a) a heavy chain complementarity determining region 1 (CDR-H1), CDR-H2, and CDR-H3 and (b) a light chain variable domain (VL) comprising (a) a light chain complementarity determining region 1 (CDR-L1), CDR-L2, and CDR-L3 of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, -ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332.

23. (canceled)

24. The multispecific binding protein of any one of claim 22, wherein the multispecific binding protein comprises a heavy chain variable region (VH) comprising an amino acid sequence at least 85%, at least 90%, at least 95%, at least 97%, at least 98%, or at least 99% identical to the VH of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332.

25. The multispecific binding protein of claim 22, wherein the multispecific binding protein comprises a light chain variable region (VL) comprising an amino acid sequence at least 85%, at least 90%, at least 95%, at least 97%, at least 98%, or at least 99% identical to the VL of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332.

26. The multispecific binding protein of any one of claim 22, wherein the multispecific binding protein comprises a heavy chain variable region (VH) comprising an amino acid sequence of the VH and/or comprising an amino acid sequence of the VL of any one of antibodies RNF43-104, RNF43-106, RNF43-107, RNF43-108, RNF43-116, RNF43-117, RNF43-123, RNF43-126, RNF43-128, RNF43-129, RNF43-130, RNF43-136, RNF43-145, RNF43-152, RNF43-156, RNF43-168, RNF43-170, RNF43-176, RNF43-177, RNF43-179, RNF43-180, RNF43-181, RNF43-186, RNF43-187, RNF43-196, RNF43-200, RNF43-201, RNF43-206, RNF43-210, RNF43-213, RNF43-217, RNF43-221, RNF43-224, RNF43-25, RNF43-31, RNF43-33, RNF43-35, RNF43-38, RNF43-41, RNF43-53, RNF43-56, RNF43-61, RNF43-67, RNF43-69, RNF43-71, RNF43-74, RNF43-75, RNF43-76, RNF43-80, RNF43-86, RNF43-90, ZNRF3-101, ZNRF3-117, ZNRF3-128, ZNRF3-131, ZNRF3-163, ZNRF3-17, ZNRF3-170, ZNRF3-171, ZNRF3-172, ZNRF3-179, ZNRF3-182, ZNRF3-195, ZNRF3-219, ZNRF3-222, ZNRF3-223, ZNRF3-231, ZNRF3-237, ZNRF3-244, ZNRF3-247, ZNRF3-253, ZNRF3-254, ZNRF3-255, ZNRF3-265, ZNRF3-269, ZNRF3-270, ZNRF3-275, ZNRF3-279, ZNRF3-287, ZNRF3-296, ZNRF3-30, ZNRF3-300, ZNRF3-301, ZNRF3-305, ZNRF3-312, ZNRF3-314, ZNRF3-322, ZNRF3-329, ZNRF3-35, ZNRF3-55, ZNRF3-6, ZNRF3-90, RNF43-1, RNF43-24, RNF43-8, RNF43-12, RNF43-20, RNF43-11, RNF43-23, RNF43-6, RNF43-5, RNF43-15, RNF43-2, RNF43-16, RNF43-14, RNF43-17, RNF43-22, RNF43-9, RNF43-21, RNF43-13, RNF43-19, ZNRF3-331, ZNRF3-333, or ZNRF3-332.

27. (canceled)

28. (canceled)

29. The multispecific binding protein of claim 16, wherein the protein is a multispecific antibody comprising a heavy chain variable region and/or a light chain variable region of a rat anti-human RNF43 B cell antibody, a rat anti-human ZNRF3 B cell antibody, a rabbit anti-human RNF43 B cell antibody, or a rabbit anti-human ZNRF3 B cell antibody.

30. The multispecific binding protein of claim 16, wherein the protein is a trispecific antibody comprising a 2+1 FvIgG or a 2+1 FabIgG format, wherein the protein binds to both ZNRF3 and RNF43 and to at least one second cell surface protein, optionally wherein the protein does not block binding of ZNRF3 or RNF43 to FZD and LRP6.

31. (canceled)

32. (canceled)

33. (canceled)

34. (canceled)

35. (canceled)

36. The multispecific binding protein of claim 1, wherein the second cell surface protein is a receptor tyrosine kinase, a growth factor receptor, a cytokine, a mucin, a Siglec receptor, or an immune checkpoint modulator, or is HER2, HER3, IGF1R, an EGFR, an FGFR, a VEGFR, a PDGFR, EpCAM, FZD, PD-L1, CTLA4, PD-1, TIM3, LAG3, TIGIT, CEACAM1, CD25, ILT-2, ILT-3, ILT-4, ILT-5, LAIR-1, PECAM-1 (CD31), PILR-alpha, SIRL-1, or SIRP-alpha.

37. (canceled)

38. (canceled)

39. An isolated nucleic acid or set of nucleic acids encoding the multispecific binding protein of claim 1.

40. A host cell comprising the nucleic acid or set of nucleic acids of claim 39.

41. A method of producing the multispecific binding protein of claim 1, comprising culturing a host cell comprising a nucleic acid or set of nucleic acids encoding the protein under conditions suitable for the expression of the protein, and optionally further recovering the protein from the host cell.

42. (canceled)

43. (canceled)

44. A pharmaceutical composition comprising the multispecific binding protein of claim 1 and a pharmaceutically acceptable carrier.

45. (canceled)

46. (canceled)

47. (canceled)

48. (canceled)

49. (canceled)

50. (canceled)

51. (canceled)

52. (canceled)

53. (canceled)

54. A method of treating cancer, an autoimmune condition, an inflammatory condition, a neurodegenerative condition, or an infectious disease in a subject in need thereof, or a method of reducing the level of a cell surface protein in a subject in need thereof, comprising administering to the subject an effective amount of the multispecific binding protein of claim 1.

55. (canceled)

56. A method of increasing an immune response in a subject in need thereof, comprising administering to the subject an effective amount of the multispecific binding protein of claim 1, optionally wherein the subject has cancer, an autoimmune condition, an inflammatory condition, a neurodegenerative condition, or an infectious disease.

57. (canceled)

58. (canceled)

59. (canceled)

60. (canceled)

61. (canceled)

62. A kit comprising the multispecific binding protein of claim 1, and/or one or more reagents for incubating the protein with a cell or tissue sample in vitro to reduce the level of a cell surface protein in the sample.

63. A method of reducing the level of a cell surface protein in a cell or tissue sample in vitro, comprising incubating the sample with the multispecific binding protein of claim 1.
